(19) United States
(12) Patent Application Publication
(10) Pub. No.: US 2017/0305922 A1
(43) Pub. Date: Oct. 26, 2017

(54) CARM1 INHIBITORS AND USES THEREOF

(71) Applicant: Epizyme, Inc., Cambridge, MA (US)

(72) Inventors: Richard Chesworth, Concord, MA (US); Oscar Miguel Moradei, Burlington, MA (US); Gideon Shapiro, Gainesville, FL (US); Lei JIn, Wellesley, MA (US); Robert E. Babine, Carlsbad, CA (US)

(73) Assignee: Epizyme, Inc., Cambridge, MA (US)

(21) Appl. No.: 15/511,503

(22) PCT Filed: Sep. 17, 2015
(86) PCT No.: PCT/US15/50712

§ 371 Date: Mar. 15, 2017

Related U.S. Application Data
(60) Provisional application No. 62/051,872, filed on Sep. 17, 2014.

(51) Int. Cl. C07D 487/10; C07D 471/04; C07D 471/10; C07D 487/04

(52) U.S. Cl. CPC C07D 487/10; C07D 487/04; C07D 471/04; C07D 471/10

(57) ABSTRACT

Provided herein are compounds of Formula (I): and pharmaceutically acceptable salts thereof, and pharmaceutical compositions thereof, wherein R1, R2a, R2b, R3 and Ring B are as defined herein, and Ring A is a group of Formula (A-i), (A-ii), or (A-iii): wherein R, R, R, R, and R are as defined herein. Compounds of the present invention are useful for inhibiting CARM1 activity. Methods of using the compounds for treating CARM1-mediated disorders are also described.

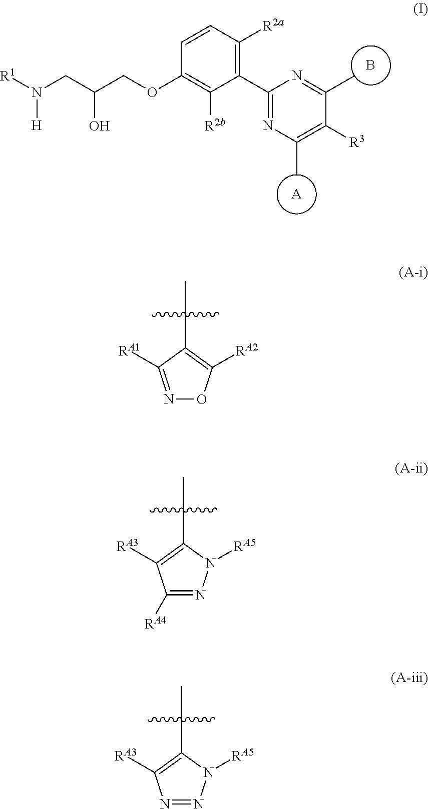

### RELATED APPLICATIONS

The present application claims priority under 35 U.S.C. §119(e) to U.S. provisional patent application, U.S. Ser. No. 62/051,872, filed Sep. 17, 2014, the entire contents of which is incorporated herein by reference.

### BACKGROUND OF THE INVENTION

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., CARM1 (co-activator-associated arginine methyltransferase 1; PRMT4)), many of which are associated with specific genetic alterations that can cause human disease.

Disease-associated chromatin-modifying enzymes play a role in diseases such as proliferative disorders, autoimmune disorders, muscular disorders, and neurological disorders. Thus, there is a need for the development of small molecules that are capable of inhibiting the activity of CARM1.

### DETAILED DESCRIPTION OF CERTAIN EMBODIMENTS

CARM1 is an attractive target for modulation given its role in the regulation of diverse biological processes. It has now been found that compounds described herein, and pharmaceutically acceptable salts and compositions thereof, are effective as inhibitors of CARM1. Such compounds have the general Formula (I):

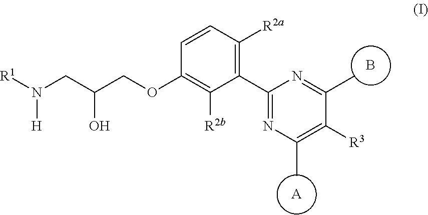

and pharmaceutically acceptable salts thereof, and pharmaceutical compositions thereof; wherein R¹, R^{2a}, R^{2b}, R³, Ring A and Ring B are as defined herein.

Pharmaceutical compositions are further provided comprising a compound described herein (e.g., a compound of Formula (I), or a pharmaceutically acceptable salt thereof) and, optionally, a pharmaceutically acceptable excipient.

In certain embodiments, compounds described herein inhibit the activity of CARM1. In certain embodiments, methods of inhibiting CARM1 are provided which comprise contacting CARM1 with an effective amount of a compound of Formula (I), or a pharmaceutically acceptable salt thereof. The CARM1 may be purified or crude, and may be present in a cell, tissue, or a subject. Thus, such methods encompass inhibition of CARM1 activity both in vitro and in vivo. In certain embodiments, the CARM1 is wild-type CARM1. In certain embodiments, the CARM1 is overexpressed. In certain embodiments, the CARM1 is a mutant. In certain embodiments, the CARM1 is in a cell. In certain embodiments, the CARM1 is in a tissue. In certain embodiments, the CARM1 is in a biological sample. In certain embodiments, the CARM1 is in an animal, e.g., a human. In some embodiments, the CARM1 is expressed at normal levels in a subject, but the subject would benefit from CARM1 inhibition (e.g., because the subject has one or more mutations in an CARM1 substrate that causes an increase in methylation of the substrate with normal levels of CARM1). In some embodiments, the CARM1 is in a subject known or identified as having abnormal CARM1 activity (e.g., overexpression). In some embodiments, the CARM1 is in a subject known or identified as having aberrant CARM1 activity. In some embodiments, a provided compound is selective for CARM1 over other methyltransferases. In certain embodiments, a provided compound is at least about 10-fold selective, at least about 20-fold selective, at least about 30-fold selective, at least about 40-fold selective, at least about 50-fold selective, at least about 60-fold selective, at least about 70-fold selective, at least about 80-fold selective, at least about 90-fold selective, or at least about 100-fold selective relative to one or more other methyltransferases.

In certain embodiments, methods of modulating gene expression or activity in a cell are provided which comprise contacting a cell with an effective amount of a compound of Formula (I), or a pharmaceutically acceptable salt thereof, or a pharmaceutical composition thereof. In certain embodiments, the cell is cultured in vitro. In certain embodiments, cell is in an animal, e.g., a human.

In certain embodiments, methods of modulating transcription in a cell are provided which comprise contacting a cell with an effective amount of a compound of Formula (I), or a pharmaceutically acceptable salt thereof, or a pharmaceutical composition thereof. In certain embodiments, the cell is cultured in vitro. In certain embodiments, the cell is in an animal, e.g., a human.

In some embodiments, methods of treating a CARM1-mediated disorder are provided which comprise administering to a subject suffering from a CARM1-mediated disorder an effective amount of a compound described herein (e.g., a compound of Formula (I), or a pharmaceutically acceptable salt thereof), or a pharmaceutical composition thereof. In certain embodiments, the CARM1-mediated disorder is a proliferative disorder. In certain embodiments, compounds described herein are useful for treating cancer. In certain embodiments, compounds described herein are useful for treating breast cancer or prostate cancer. In certain embodiments, the CARM1-mediated disorder is a metabolic disorder.

Compounds described herein are also useful for the study of CARM1 in biological and pathological phenomena, the study of intracellular signal transduction pathways mediated by CARM1, and the comparative evaluation of new CARM1 inhibitors.

This application refers to various issued patent, published patent applications, journal articles, and other publications, all of which are incorporated herein by reference.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₃ alkyl” is intended to encompass, C₁, C₂, C₃, C₁₋₃, C₁₋₂, and C₂₋₃ alkyl.

“Alkyl” refers to a radical of a straight-chain or branched saturated hydrocarbon group having from 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₃ alkyl groups include methyl (C₁), ethyl (C₂), n-propyl (C₃), and isopropyl (C₃). Alkyl groups may be substituted or unsubstituted as described herein.

“Haloalkyl” refers to an alkyl group, as defined herein, substituted with one or more halogen atoms, e.g., 1, 2, 3, 4, 5, 6, or 7 halogen atoms independently selected from the group consisting of fluoro, bromo, chloro, and iodo. Haloalkyl encompasses perhaloalkyl as defined herein. “Perhaloalkyl” refers to a substituted alkyl group as defined herein wherein all of the hydrogen atoms are independently replaced by a halogen. In some embodiments, at least one of the hydrogen atoms is replaced with fluoro. In some embodiments, at least one of the hydrogen atoms is replaced with chloro. Examples of perhaloalkyl groups include —CF₃, —CF₂CF₃, —CF₂CF₂CF₃, —CCl₃, —CFCl₂, —CF₂Cl, and the like. Examples of haloalkyl groups include all of the aforementioned perhaloalkyl groups, as well as groups such as —CH₂F, —CHF₂, —CH₂Cl, CHCl₂, —CH₂CF₃, —CH₂CHF₂, —CH₂CH₂F, —CH₂CCl₃, —CH₂CHCl₂, —CH₂CH₂Cl, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, —CH(CH₃)CF₃, and the like.

“Alkenyl” refers to a radical of a straight-chain or branched hydrocarbon group having from 2 to 3 carbon atoms and one carbon-carbon double bond (“C₂₋₃ alkenyl”). In some embodiments, an alkenyl group has 2 carbon atoms (“C₂ alkenyl”). In some embodiments, an alkenyl group has 3 carbon atoms (“C₃ alkenyl”). Examples of C₂₋₃ alkenyl groups include ethenyl (C₂), 1-propenyl (C₃), and 2-propenyl (C₃). Alkenyl groups may be substituted or unsubstituted as described herein.

“Alkynyl” refers to a radical of a straight-chain or branched hydrocarbon group having from 2 to 3 carbon atoms and one carbon-carbon triple bond (“C₂₋₃ alkynyl”). In some embodiments, an alkynyl group has 2 carbon atoms (“C₂ alkynyl”). In some embodiments, an alkynyl group has 3 carbon atoms (“C₃ alkynyl”). Examples of C₂₋₃ alkynyl groups include, without limitation, ethynyl (C₂), 1-propynyl (C₃), and 2-propynyl (C₃). Alkynyl groups may be substituted or unsubstituted as described herein.

“Carbocyclyl” or “carbocyclic” refers to a radical of a non-aromatic monocyclic hydrocarbon group having from 3 to 6 ring carbon atoms (“C₃₋₆ carbocyclyl”) and zero heteroatoms in the non-aromatic ring system. In some embodiments, a carbocyclyl group has 3 to 4 ring carbon atoms (“C₃₋₄ carbocyclyl”). In some embodiments, a carbocyclyl group has 3 to 5 ring carbon atoms (“C₃₋₅ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like.

“Heterocyclyl” or “heterocyclic” refers to a radical of a 4-6 membered monocyclic non-aromatic ring system having ring carbon atoms and 1, 2, or 3 ring heteroatoms, wherein each heteroatom is independently selected from nitrogen, oxygen, and sulfur (“4-6 membered heterocyclyl”). In heterocyclyl groups that contain one or more nitrogen atoms, the point of attachment can be a carbon or nitrogen atom, as valency permits. In some embodiments, a heterocyclyl group is a 4-membered monocyclic non-aromatic ring system having ring carbon atoms and 1 ring heteroatom, wherein each heteroatom is independently selected from nitrogen, oxygen, and sulfur (“4-membered heterocyclyl”). In some embodiments, a heterocyclyl group is a 5-membered monocyclic non-aromatic ring system having ring carbon atoms and 1, 2, or 3 ring heteroatoms, wherein each heteroatom is independently selected from nitrogen, oxygen, and sulfur (“5-membered heterocyclyl”). In some embodiments, a heterocyclyl group is a 6-membered monocyclic non-aromatic ring system having ring carbon atoms and 1, 2, or 3 ring heteroatoms, wherein each heteroatom is independently selected from nitrogen, oxygen, and sulfur (“6-membered heterocyclyl”). Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, pyrrolyl-2,5-dione, and pyrrolidin-2-one. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, as defined herein.

In general, the term “substituted” means that at least one hydrogen present on a group (e.g., a carbon or nitrogen atom) is replaced with a substituent as defined herein and results in a stable compound, e.g., a compound which does not spontaneously undergo transformation such as by rearrangement, cyclization, elimination, or other reaction. Unless otherwise indicated, a “substituted” group may have a substituent at one or more substitutable positions of the group, and when more than one position in any given structure is substituted, the substituent may be the same or different at each position.

“Halo” or “halogen” refers to fluorine (fluoro, —F), chlorine (chloro, —Cl), bromine (bromo, —Br), or iodine (iodo, —I).

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

A “subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), rodents (e.g., rats and/or mice), reptiles, amphibians, and fish. In certain embodiments, the non-human animal is a mammal. The non-human animal may be a male or female at any stage of development. A non-human animal may be a transgenic animal.

“Condition,” “disease,” and “disorder” are used interchangeably herein.

“Treat,” “treating” and “treatment” encompasses an action that occurs while a subject is suffering from a condition which reduces the severity of the condition or retards or slows the progression of the condition (“therapeutic treatment”). “Treat,” “treating” and “treatment” also encompasses an action that occurs before a subject begins to suffer from the condition and which inhibits or reduces the severity of the condition (“prophylactic treatment”).

An “effective amount” of a compound refers to an amount sufficient to elicit the desired biological response, e.g., treat the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, the term “methyltransferase” represents transferase class enzymes that are able to transfer a methyl group from a donor molecule to an acceptor molecule, e.g., an amino acid residue of a protein or a nucleic base of a DNA molecule. Methytransferases typically use a reactive methyl group bound to sulfur in S-adenosyl methionine (SAM) as the methyl donor. In some embodiments, a methyltransferase described herein is a protein methyltransferase. In some embodiments, a methyltransferase described herein is a histone methyltransferase. Histone methyltransferases (HMT) are histone-modifying enzymes, (including histone-lysine N-methyltransferase and histone-arginine N-methyltransferase), that catalyze the transfer of one or more methyl groups to lysine and arginine residues of histone proteins. In certain embodiments, a methyltransferase described herein is a histone-arginine N-methyltransferase.

As generally described above, provided herein are compounds useful as CARM1 inhibitors. In some embodiments, the present disclosure provides a compound of Formula (I):

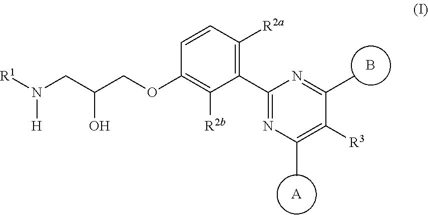

or pharmaceutically acceptable salt thereof;
wherein:

R¹ is hydrogen, —CHO, or unsubstituted C₁₋₃alkyl;

each instance of R^{2a} and R^{2b} is independently hydrogen, halogen, unsubstituted C₁₋₃alkyl, or C₁₋₃haloalkyl;

R³ is unsubstituted C₁₋₃alkyl, C₁₋₃haloalkyl, or halogen;

Ring A is of formula (A-i), (A-ii), or (A-iii):

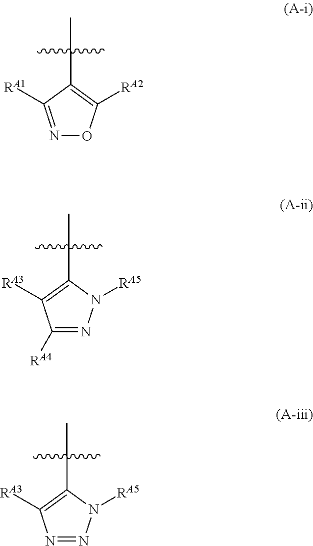

wherein:
each instance of R^{A1} and R^{A2} is independently unsubstituted C₁₋₃alkyl, C₁₋₃haloalkyl, or unsubstituted cyclopropyl;
R^{A3} is unsubstituted C₁₋₃alkyl, C₁₋₃haloalkyl, halogen, or —CN;
R^{A4} is hydrogen, unsubstituted C₁₋₃alkyl, C₁₋₃haloalkyl, halogen, or —CN; and
R^{A5} is unsubstituted C₁₋₃alkyl or C₁₋₃haloalkyl;

Ring B is any one of formula (i) to (xxviii):

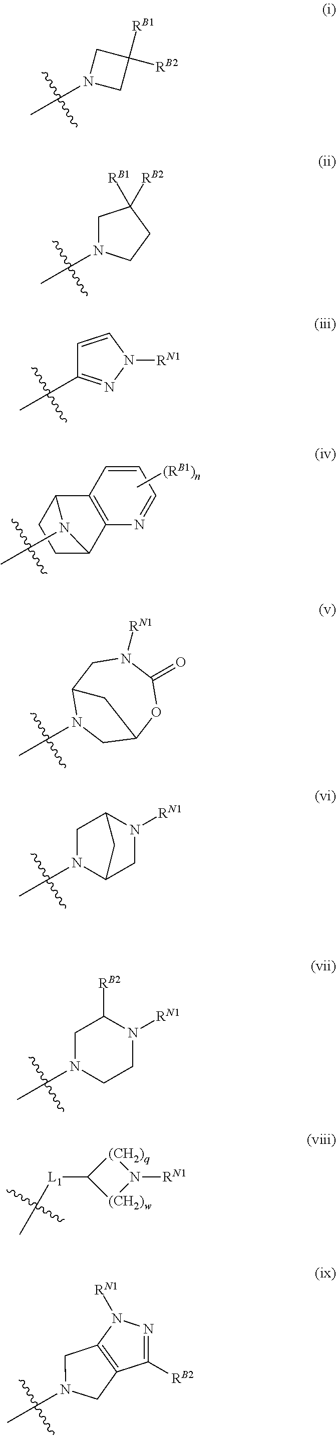

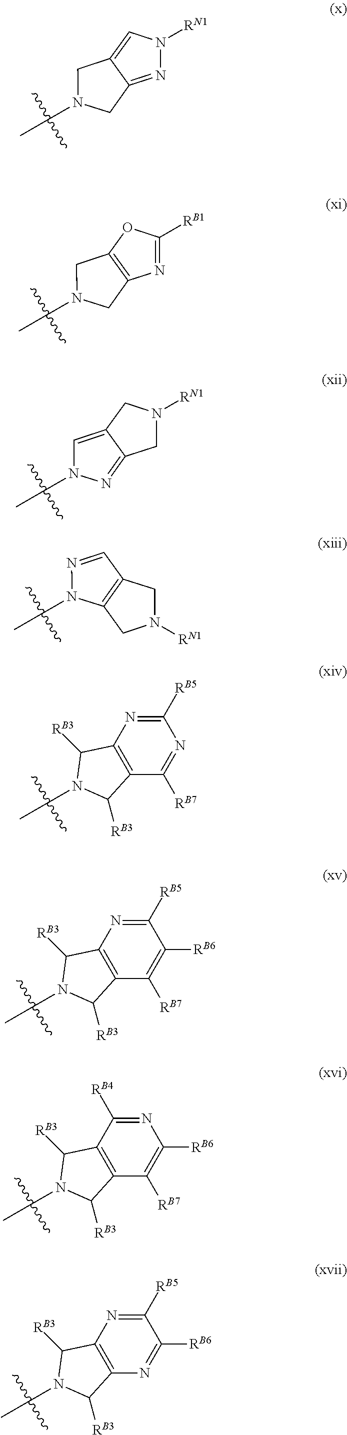

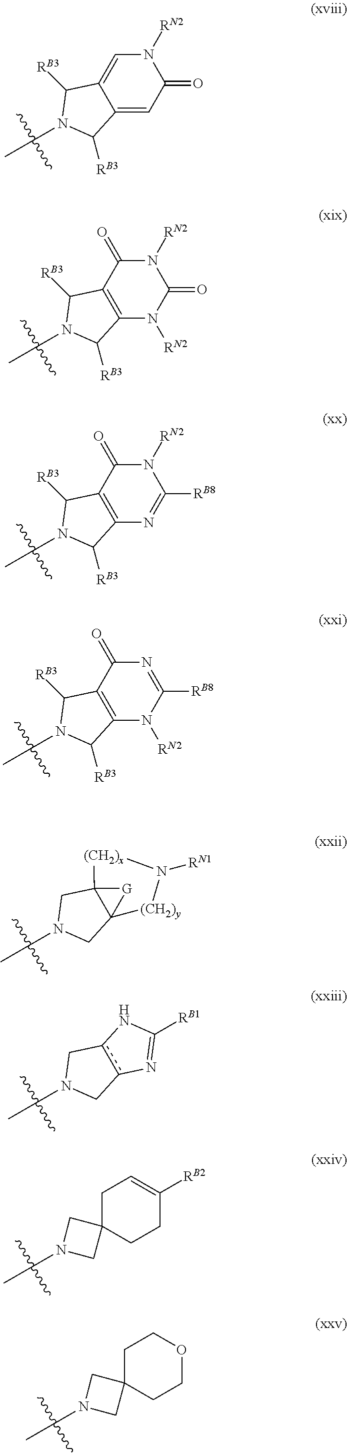

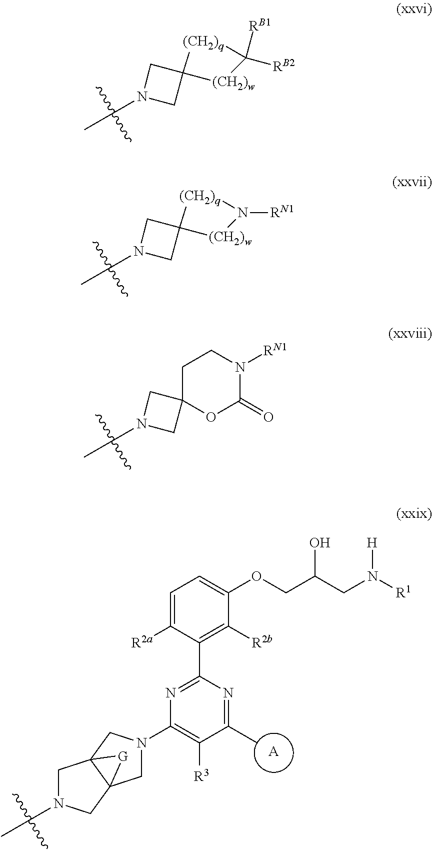

wherein:
q is 1, 2, or 3 and w is 1; or q is 2 and w is 0 or 2;
x is 1 and y is 1 or 2;
n is 0, 1, or 2;
L₁ is —NH—, substituted or unsubstituted C₂alkylene, substituted or unsubstituted C₂alkenylene, or substituted or unsubstituted C₂alkynylene;
R^{N1} is substituted or unsubstituted C₁₋₃alkyl, C₁₋₃haloalkyl substituted or unsubstituted C₃₋₆ carbocyclyl, substituted or unsubstituted 4- to 6-membered heterocyclyl, —C(═O)R^{N1A}, —C(═O)N(R^{N1A})(R^{N1B}), —C(═O)OR^{N1A}, or —S(O)₂R^{N1A}; wherein:
R^{N1A} is substituted or unsubstituted C₁₋₃alkyl, C₁₋₃haloalkyl, substituted or unsubstituted C₃₋₆ carbocyclyl, or substituted or unsubstituted 4- to 6-membered heterocyclyl;
R^{N1B} is hydrogen, substituted or unsubstituted C₁₋₃alkyl, C₁₋₃haloalkyl, substituted or unsubstituted C₃₋₆ carbocyclyl, or substituted or unsubstituted 4- to 6-membered heterocyclyl; or
R^{N1A} and R^{N1B} are joined to form a substituted or unsubstituted 4- to 6-membered heterocyclyl; or
each instance of R^{N2} and R^{B8} is independently substituted or unsubstituted C₁₋₃alkyl or C₁₋₃haloalkyl, or R^{N2} and R^{B8} are joined to form a substituted or unsubstituted 5- to 6-membered ring;
R^{B1} is substituted or unsubstituted C₁₋₃alkyl, C₁₋₃haloalkyl, halogen, —CN, —OR^{B1B}, —SR^{B1B}, —N(R^{B1A})(R^{B1B}), substituted or unsubstituted C₃₋₆ carbocyclyl, substituted or unsubstituted 4- to 6-membered heterocyclyl, —C(═O)R^{B1A}, —C(═O)N(R^{B1A})(R^{B1B}), —C(═O)OR^{B1A}, —S(O)₂R^{B1A}, —OC(═O)R^{B1A}, —OC(═O)N(R^{B1A})(R^{B1B}), —OC(═O)OR^{B1A}, —NR^{B1B}C(═O)R^{B1A}, —NR^{B1B}C(═O)N(R^{B1A})(R^{B1B}), or —NR^{B1B}C(═O)OR^{B1A};
wherein:
R^{B1A} is substituted or unsubstituted C₁₋₃alkyl, C₁₋₃haloalkyl, substituted or unsubstituted C₃₋₆ carbocyclyl, or substituted or unsubstituted 4- to 6-membered heterocyclyl; and
R^{B1B} is hydrogen, substituted or unsubstituted C₁₋₃alkyl, C₁₋₃haloalkyl, substituted or unsubstituted C₃₋₆ carbocyclyl, or substituted or unsubstituted 4- to 6-membered heterocyclyl; or
R^{B1A} and R^{B1B} are joined to form a substituted or unsubstituted 4- to 6-membered heterocyclyl;
R^{B2} is hydrogen, halogen, —OR^{B2A}, substituted or unsubstituted C₁₋₃alkyl, or C₁₋₃haloalkyl, wherein R^{B2A} is substituted or unsubstituted C₁₋₃alkyl or C₁₋₃haloalkyl; or
R^{B1} and R^{B2} are joined to form a substituted or unsubstituted 4- to 6-membered heterocyclyl;
each instance of R^{B3} is independently hydrogen, unsubstituted C₁₋₃alkyl, or C₁₋₃haloalkyl, provided at least one instance of R^{B3} is hydrogen;
each instance of R^{B4}, R^{B5}, R^{B6}, and R^{B7} is independently hydrogen, substituted or unsubstituted C₁₋₃alkyl, C₁₋₃haloalkyl, halogen, —CN, —OR^{B4B}, —SR^{B4B}, —N(R^{B4A})(R^{B4B}), substituted or unsubstituted C₃₋₆ carbocyclyl, substituted or unsubstituted 4- to 6-membered heterocyclyl, —C(═O)R^{B4A}, —C(═O)N(R^{B4A})(R^{B4B}), —C(═O)OR^{B4A}, —S(O)₂R^{B4A}, —OC(═O)R^{B4A}, —OC(═O)N(R^{B4A})(R^{B4B}), —OC(═O)OR^{B4A}, —NR^{B4B}C(═O)R^{B4A}, —NR^{B4B}C(═O)N(R^{B4A})(R^{B4B}), or —NR^{B4B}C(═O)OR^{B4A};
wherein:
R^{B4A} is substituted or unsubstituted C₁₋₃alkyl, C₁₋₃haloalkyl, substituted or unsubstituted C₃₋₆ carbocyclyl, or substituted or unsubstituted 4- to 6-membered heterocyclyl; and
R^{B4B} is hydrogen, substituted or unsubstituted C₁₋₃alkyl, C₁₋₃haloalkyl, substituted or unsubstituted C₃₋₆ carbocyclyl, or substituted or unsubstituted 4- to 6-membered heterocyclyl; or
R^{B4A} and R^{B4B} are joined to form a substituted or unsubstituted 4- to 6-membered heterocyclyl;
   and
wherein [symbol] represents a single or double bond; and
further wherein

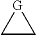

represents a single or double bond or G is —CH₂—;

wherein each instance of substituted independently refers to substitution with 1, 2, or 3 R^{C1} groups, as valency permits,

and wherein:

each instance of R^{C1} is independently unsubstituted C₁₋₃alkyl, C₁₋₃haloalkyl, halogen, —CN, —OR^{C1B}, —SR^{C1B}, —N(R^{C1A})(R^{C1B}), —C(═O)R^{C1A}, —C(═O)N(R^{C1A})(R^{C1B}), —C(═O)OR^{C1A}, —S(O)₂R^{C1A}, —OC(═O)R^{C1A}, —OC(═O)N(R^{C1A})(R^{C1B}), —OC(═O)OR^{C1A}, —NR^{C1B}C(═O)R^{C1A}, —NR^{C1B}C(═O)N(R^{C1A})(R^{C1B}), or —NR^{C1B}C(═O)OR^{C1A};

wherein:

R^{C1A} is unsubstituted C₁₋₃alkyl, C₁₋₃haloalkyl, C₃₋₆carbocylyl unsubstituted or substituted with 1 or 2 R^{D1} groups; or 4-6 membered heterocyclyl unsubstituted or substituted with 1 or 2 R^{D1} groups; and

R^{C1B} is hydrogen, unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups; or

R^{C1A} and R^{C1B} are joined to form an 4- to 6-membered heterocyclyl unsubstituted or substituted with 1 or 2 R^{D1} groups; and

wherein:

each instance of R^{D1} is independently halogen, —CN, —OR^{D1A}, unsubstituted C₁₋₃alkyl, or C₁₋₃haloalkyl, wherein R^{D1A} is hydrogen, unsubstituted C₁₋₃alkyl, or C₁₋₃haloalkyl.

In certain embodiments, the compound of Formula (I) is a stereoisomer of Formula:

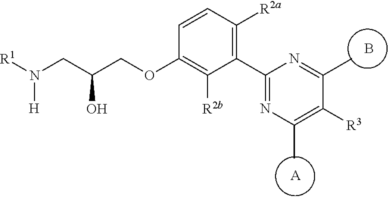

or a pharmaceutically acceptable salt thereof.

In certain embodiments, the compound of Formula (I) is a stereoisomer of Formula:

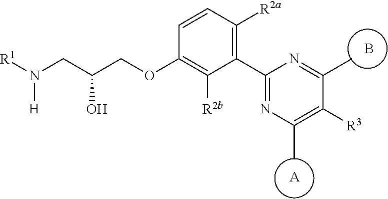

or a pharmaceutically acceptable salt thereof.
(I) Groups R¹, R², and R³

As generally defined herein, R¹ is hydrogen, —CHO, or unsubstituted C₁₋₃alkyl.

In certain embodiments, R¹ is hydrogen. In certain embodiments, R¹ is —CHO. In certain embodiments, R¹ is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂).

Furthermore, as generally defined herein, each instance of R^{2a} and R^{2b} is independently hydrogen, halogen, unsubstituted C₁₋₃alkyl, or C₁₋₃haloalkyl.

In certain embodiments, at least one instance of R^{2a} and R^{2b} is hydrogen. In certain embodiments, each instance of R^{2a} and R^{2b} is hydrogen.

In certain embodiments, at least one instance of R^{2a} and R^{2b} is halogen, i.e., at least one instance of R^{2a} and R^{2b} is —F, —Cl, —Br, or —I. In certain embodiments, R^{2a} is halogen and R^{2b} is halogen, i.e., each instance of R^{2a} and R^{2b} is independently —F, —Cl, —Br, or —I. In certain embodiments, at least one instance of R^{2a} and R^{2b} is —F or —Cl. In certain embodiments, R^{2a} is —F or —Cl. In certain embodiments, R^{2b} is —F or —Cl. In certain embodiments, R^{2a} is —Cl and R^{2b} is —Cl. In certain embodiments, R^{2a} is —F and R^{2b} is —F.

In certain embodiments, at least one instance of R^{2a} and R^{2b} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, at least one instance of R^{2a} and R^{2b} is —CH₃.

In certain embodiments, at least one instance of R^{2a} and R^{2b} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, at least one instance of R^{2a} and R^{2b} is —CF₃. In certain embodiments, R^{2a} is —CF₃. In certain embodiments, R^{2b} is —CF₃.

In certain embodiments, R^{2b} is hydrogen and R^{2a} is halogen, unsubstituted C₁₋₃alkyl, or C₁₋₃haloalkyl. In certain embodiments, R^{2b} is hydrogen and R^{2a} is halogen, i.e., R^{2b} is hydrogen and R^{2a} is —F, —Cl, —Br, or —I. In certain embodiments, R^{2b} is hydrogen and R^{2a} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{2b} is hydrogen and R^{2a} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{2b} is hydrogen and R^{2a} is —Cl. In certain embodiments, R^{2b} is hydrogen and R^{2a} is —F. In certain embodiments, R^{2b} is hydrogen and R^{2a} is —CF₃.

In certain embodiments, R^{2a} is hydrogen and R^{2b} is halogen, unsubstituted C₁₋₃alkyl, or C₁₋₃haloalkyl. In certain embodiments, R^{2a} is hydrogen and R^{2b} is halogen, i.e., R^{2a} is hydrogen and R^{2b} is —F, —Cl, —Br, or —I. In certain embodiments, R^{2a} is hydrogen and R^{2b} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{2a} is hydrogen and R^{2b} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{2a} is hydrogen and R^{2b} is —CF₃.

In certain embodiments, R^{2a} is hydrogen and R^{2b} is —Cl. In certain embodiments, R^{2a} is hydrogen and R^{2b} is —F. In certain embodiments, R^{2a} is hydrogen and R^{2b} is —CF₃.

Furthermore, as generally defined herein, R³ is unsubstituted C₁₋₃alkyl, C₁₋₃haloalkyl, or halogen. In certain embodiments, R³ is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R³ is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R³ is —CH₃. In certain embodiments, R³ is halogen, i.e., —F, —Cl, —Br, or —I. In certain embodiments, R³ is —F or —Cl.

Various combinations of R^{2a}, R^{2b}, and R³ are contemplated herein.

For example, in certain embodiments, each of R^{2a} and R³ is the same group. In certain embodiments, R^{2a} and R³ are different groups. In certain embodiments, each of R^{2a} and R³ is halogen, e.g., R^{2a} is —Cl and R³ is —Cl, or R^{2a} is —F and R³ is —F, or R^{2a} is —Cl and R³ is —F, or R^{2a} is —F and R³ is —Cl. In certain embodiments, R^{2a} is halogen and R³ is unsubstituted C₁₋₃alkyl, e.g., wherein R^{2a} is —Cl and R³ is —CH₃, or R^{2a} is —F and R³ is —CH₃. In certain embodiments, R^{2a} is C₁₋₃haloalkyl and R³ is unsubstituted C₁₋₃alkyl, e.g., R^{2a} is —CF₃ and R³ is —CH₃. In certain embodiments, R^{2a} is hydrogen and R³ is unsubstituted C₁₋₃alkyl, e.g., wherein R^{2a} is hydrogen and R³ is —CH₃.

In certain embodiments, R^{2a} is halogen (e.g., —F or —Cl), R^{2b} is hydrogen, and R³ is unsubstituted C₁₋₃alkyl (e.g., —CH₃). In certain embodiments, R^{2a} is —Cl, R^{2b} is hydrogen, and R³ is —CH₃, or R^{2a} is —F, R^{2b} is hydrogen, and R³ is —CH₃, to provide a compound of Formulae:

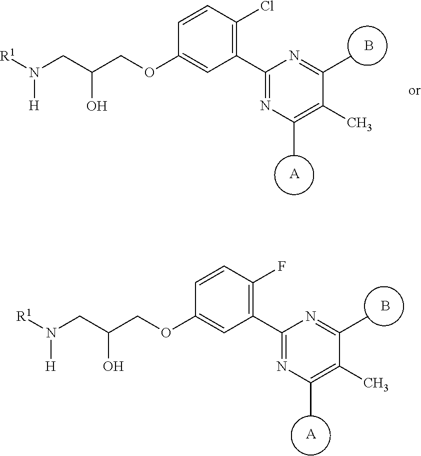

or a pharmaceutically acceptable salt thereof.

In certain embodiments, R^{2a} is halogen (e.g., —F or —Cl), R^{2b} is hydrogen, and R³ is halogen (e.g., —F or —Cl). In certain embodiments, R^{2a} is —Cl, R^{2b} is hydrogen, and R³ is —Cl, or R^{2a} is —F, R^{2b} is hydrogen, and R³ is —F, to provide a compound of Formulae:

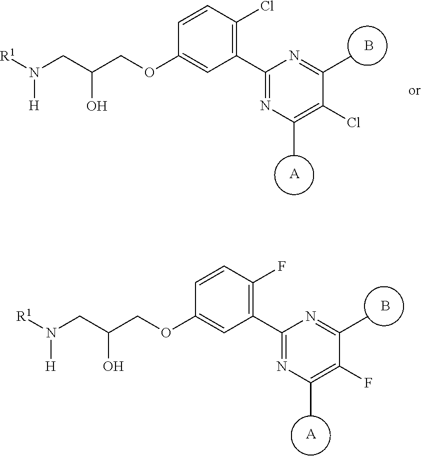

or a pharmaceutically acceptable salt thereof.

In certain embodiments, R^{2a} is C₁₋₃haloalkyl (e.g., —CF₃), R^{2b} is hydrogen, and R³ is unsubstituted C₁₋₃alkyl (e.g., —CH₃). In certain embodiments, R^{2a} is —CF₃, R^{2b} is hydrogen, and R³ is —CH₃ to provide a compound of Formula:

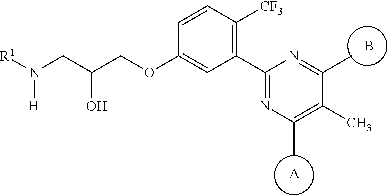

or a pharmaceutically acceptable salt thereof.

In certain embodiments, each of R^{2a} and R^{2b} is hydrogen, and R³ is unsubstituted C₁₋₃alkyl (e.g., —CH₃). In certain embodiments, R^{2a} is hydrogen, R^{2b} is hydrogen, and R³ is —CH₃ to provide a compound of Formula:

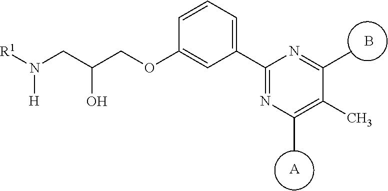

or a pharmaceutically acceptable salt thereof.

In certain embodiments, each of R^{2a}, R^{2b}, and R³ is halogen (e.g., —F or —Cl). For example, in certain embodiments, each of R^{2a}, R^{2b}, and R³ is —Cl, or each of R^{2a}, R^{2b}, and R³ is —F, to provide a compound of Formulae:

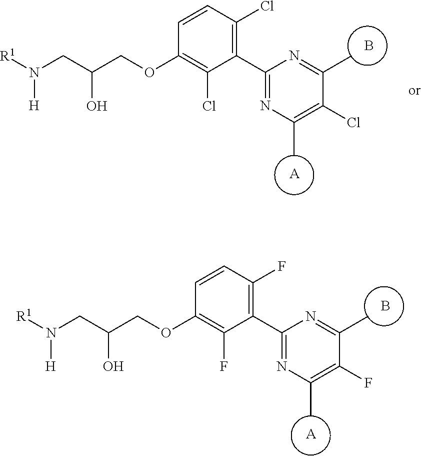

or a pharmaceutically acceptable salt thereof.

In certain embodiments, each of R^{2a} and R^{2b} is independently halogen (e.g., —F or —Cl), R³ is unsubstituted C₁₋₃alkyl (e.g., —CH₃). In certain embodiments, R^{2a} is —Cl, R^{2b} is —Cl, and R³ is —CH₃, or R^{2a} is —F, R^{2b} is —F, and R³ is —CH₃, provide a compound of Formulae:

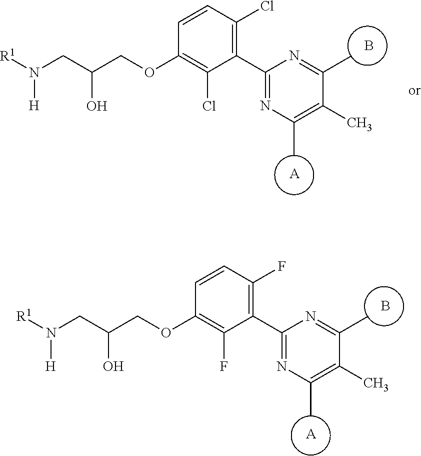

As generally defined herein, Ring A is of formula (A-i), (A-ii), or (A-iii):

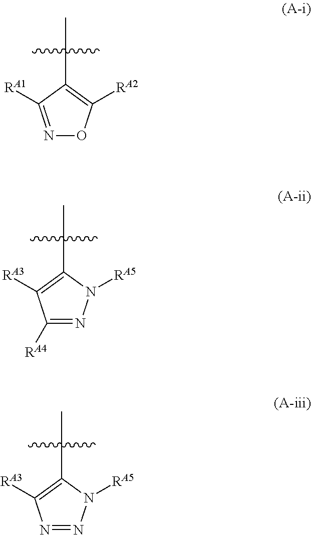

wherein:

each instance of R^{A1} and R^{A2} is independently unsubstituted C₁₋₃alkyl or C₁₋₃haloalkyl;

R^{A3} is unsubstituted C₁₋₃alkyl, C₁₋₃haloalkyl, halogen, or —CN;

R^{A4} is hydrogen, unsubstituted C₁₋₃alkyl, C₁₋₃haloalkyl, halogen, or —CN; and

R^{A5} is unsubstituted C₁₋₃alkyl or C₁₋₃haloalkyl.

### (II) Ring A

In certain embodiments, Ring A is of Formula (A-i):

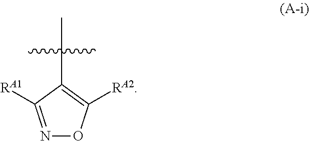

wherein each instance of R^{A1} and R^{A2} is independently unsubstituted C₁₋₃alkyl, C₁₋₃haloalkyl, or unsubstituted cyclopropyl.

In certain embodiments, at least one instance of R^{A1} and R^{A2} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, at least one of R^{A1} and R^{A2} is —CH₃. In certain embodiments, at least one of R^{A1} and R^{A2} is —CH₂CH₃.

In certain embodiments, at least one instance of R^{A1} and R^{A2} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, at least one instance of R^{A1} and R^{A2}—CF₃.

In certain embodiments, at least one of R^{A1} and R^{A2} is unsubstituted cyclopropyl.

In certain embodiments, R^{A1} and R^{A2} are the same group, e.g., in certain embodiments, R^{A1} and R^{A2} are each —CH₃. However, in certain embodiments, R^{A1} and R^{A2} are different groups, e.g., in certain embodiments, R^{A1} is —CH₃ and R^{A2} is —CH₂CH₃, or in certain embodiments, R^{A1} is —CH₂CH₃ and R^{A2} is —CH₃, or in certain embodiments, R^{A1} is unsubstituted cyclopropyl and R^{A2} is —CH₃, or in certain embodiments, R^{A2} is unsubstituted cyclopropyl and R^{A1} is —CH₃.

In certain embodiments, Ring A is selected from the group consisting of:

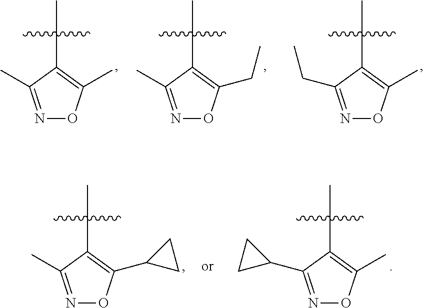

In certain embodiments, Ring A is of Formula (A-ii):

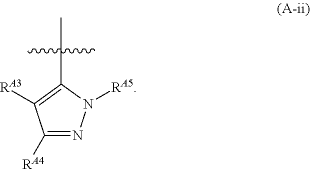

In certain embodiments, R^{A3} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{A3} is C₁₋₃haloalkyl, e.g., C₁ haloalkyl C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{A3} is halogen, i.e., —F, —Cl, —Br, or —I. In certain embodiments, R^{A3} is —CN. In certain embodiments, R^{A3} is —CN provided R^{A4} is is not also —CN.

In certain embodiments, R^{A4} is hydrogen. In certain embodiments, R^{A4} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{A4} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{A4} is halogen, i.e., —F, —Cl, —Br, or —I. In certain embodiments, R^{A4} is —CN. In certain embodiments, R^{A4} is —CN provided R^{A3} is is not also —CN.

In certain embodiments, R^{A5} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{A5} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃).

Various combinations of R^{A3}, R^{A4}, and R^{A5} are contemplated herein.

For example, in certain embodiments, R^{A3} is halogen, —CN, unsubstituted C₁₋₃alkyl, or C₁₋₃haloalkyl, and R^{A4} is hydrogen. In certain embodiments, R^{A3} is halogen (i.e., —F, —Cl, —Br, or —I), and R^{A4} is hydrogen. In certain embodiments, R^{A3} is —CN and R^{A4} is hydrogen. In certain embodiments, R^{A3} is unsubstituted C₁₋₃alkyl (e.g., —CH₃, —CH₂CH₃) and R^{A4} is hydrogen. Furthermore, in certain embodiments, in any of the above recited instances, R^{A5} is unsubstituted C₁₋₃alkyl (e.g., —CH₃, —CH₂CH₃).

In certain embodiments, R^{A3} is unsubstituted C₁₋₃alkyl or C₁₋₃haloalkyl and R^{A4} is halogen or —CN. In certain embodiments, R^{A4} is unsubstituted C₁₋₃alkyl or C₁₋₃haloalkyl and R^{A3} is halogen or —CN. In certain embodiments, R^{A3} is unsubstituted C₁₋₃alkyl (e.g., —CH₃, —CH₂CH₃) and R^{A4} is halogen (i.e., —F, —Cl, —Br, or —I). In certain embodiments, R^{A3} is unsubstituted C₁₋₃alkyl (e.g., —CH₃, —CH₂CH₃) and R^{A4} is —CN. Furthermore, in certain embodiments, in any of the above recited instances, R^{A5} is unsubstituted C₁₋₃alkyl (e.g., —CH₃, —CH₂CH₃).

In certain embodiments, Ring A is selected from the group consisting of:

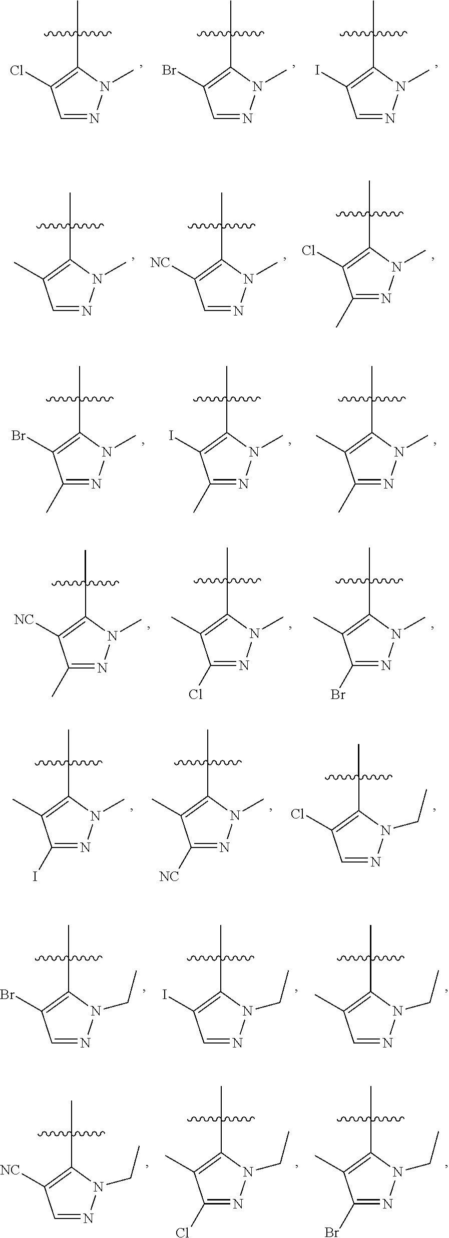

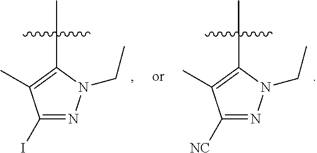

In certain embodiments, Ring A is of Formula (A-ii):

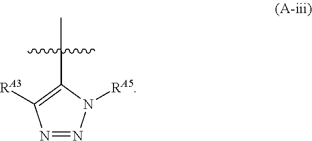

In certain embodiments, R^{A3} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{A3} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{A3} is halogen, i.e., —F, —Cl, —Br, or —I.

In certain embodiments, R^{A5} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{A5} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃).

Various combinations of R^{A3} and R^{A5} are contemplated herein.

For example, in certain embodiments, R^{A3} is halogen, —CN, unsubstituted C₁₋₃alkyl, or C₁₋₃haloalkyl, and R^{A5} is unsubstituted C₁₋₃alkyl (e.g., —CH₃, —CH₂CH₃). In certain embodiments, R^{A3} is halogen (i.e., —F, —Cl, —Br, or —I), and R^{A5} is unsubstituted C₁₋₃alkyl (e.g., —CH₃, —CH₂CH₃). In certain embodiments, R^{A3} is —CN and R^{A5} is unsubstituted C₁₋₃alkyl (e.g., —CH₃, —CH₂CH₃). In certain embodiments, R^{A3} is unsubstituted C₁₋₃alkyl (e.g., —CH₃, —CH₂CH₃) and R^{A5} is unsubstituted C₁₋₃alkyl (e.g., —CH₃, —CH₂CH₃). In certain embodiments, R^{A3} and R^{A5} are the same group. In certain embodiments, R^{A3} and R^{A5} are different groups.

In certain embodiments, Ring A is:

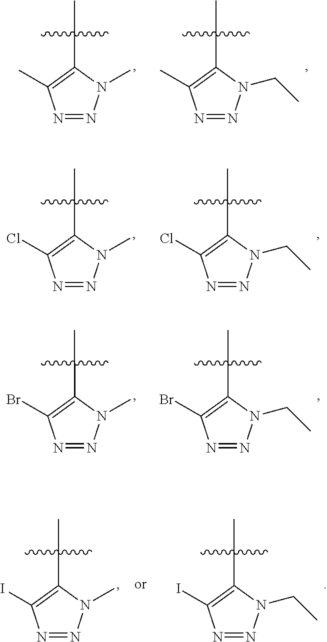

Various combinations of Ring A, R¹, R^{2a}, and R^{2b}, are contemplated herein.

For example, in certain embodiments, wherein Ring A is of Formula (A-i), (A-ii), or (A-iii), R¹ is —CH₃, and each R^{2a} and R^{2b} is hydrogen, provided is a compound of Formula:

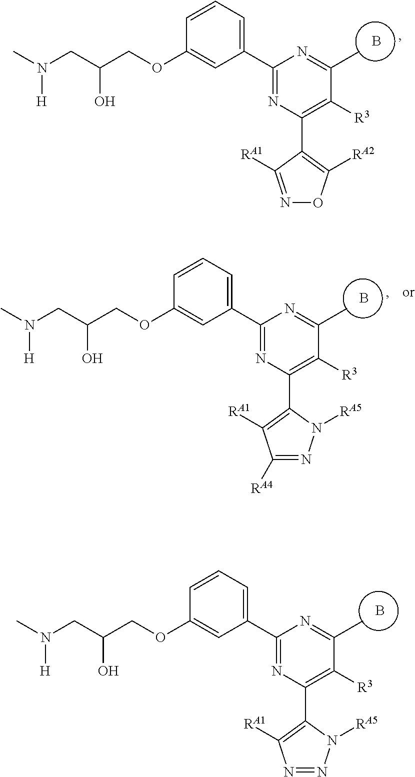

or a pharmaceutically acceptable salt thereof.

In certain embodiments, wherein Ring A is of Formula (A-i), (A-ii), or (A-iii), R¹ is —CH₃, R^{2a} is —Cl, and R^{2b} is hydrogen, provided is a compound of Formula:

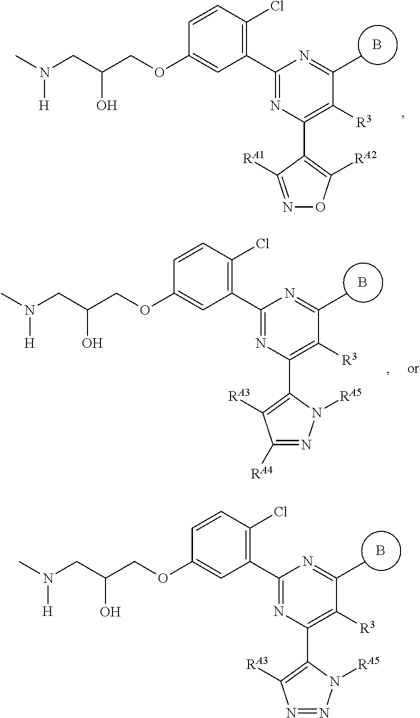

or a pharmaceutically acceptable salt thereof.

In certain embodiments, wherein Ring A is of Formula (A-i), (A-ii), or (A-iii), R¹ is —CH₃, R^{2a} is —F, and R^{2b} is hydrogen, provided is a compound of Formulae:

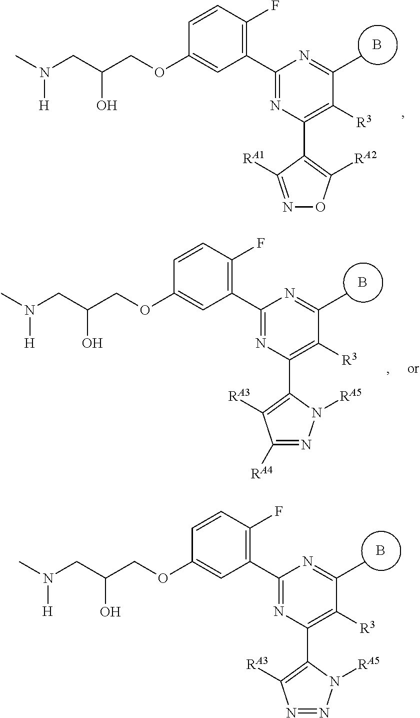

or a pharmaceutically acceptable salt thereof.

In certain embodiments, wherein Ring A is of Formula (A-i), (A-ii), or (A-iii), R¹ is —CH₃, R^{2a} is —CF₃, and R^{2b} is hydrogen, provided is a compound of Formulae:

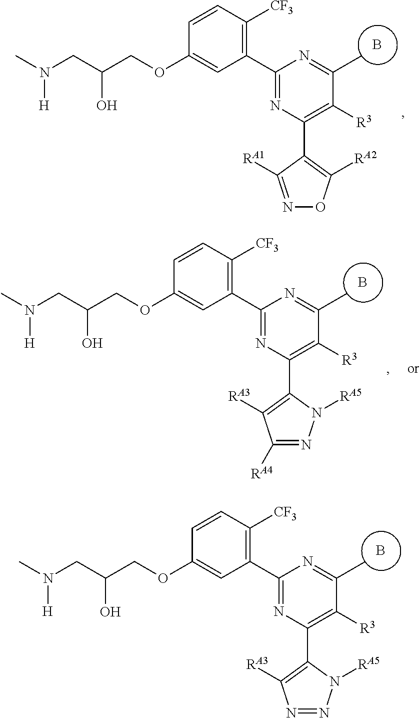

or a pharmaceutically acceptable salt thereof.

In certain embodiments, wherein Ring A is of Formula (A-i), (A-ii), or (A-iii), R¹ is —CH₃, R^{2a} is —Cl, and R^{2b} is —Cl, provided is a compound of Formulae:

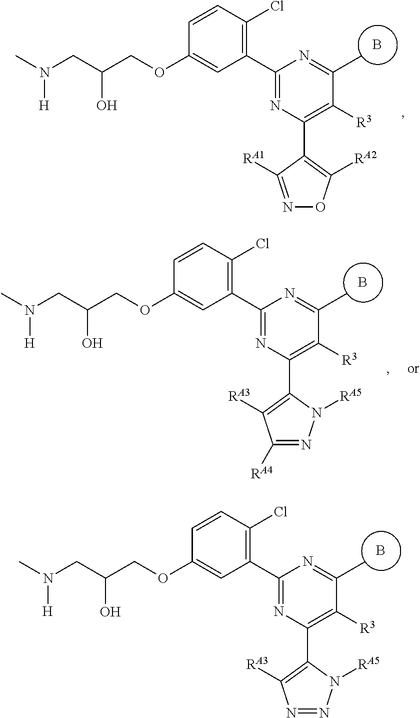

or a pharmaceutically acceptable salt thereof.
(III) Ring B Groups: Substitution by R^{C1} and R^{D1}

It is generally understood, as described herein, that each instance of “substituted” preceding a group refers to a group, e.g., substituted C₂alkylene, substituted C₂alkenylene, or substituted C₂alkynylene in the instance of L₁, and substituted C₁₋₃alkyl, substituted C₃₋₆ carbocyclyl, substituted 4- to 6-membered heterocyclyl, and substituted 5- to 6-membered ring, in the instance of various Ring B recitations, refers to a group substituted with 1, 2, or 3 R^{C1} groups, as valency permits. In certain embodiments, such groups are substituted with 1 or 2 R^{C1} groups.

As generally defined herein, each instance of R^{C1} is independently unsubstituted C₁₋₃alkyl, C₁₋₃haloalkyl, halogen, —CN, —OR^{C1B}, —SR^{C1B}, —N(R^{C1A})(R^{C1B}), —C(═O)R^{1A}, —C(═O)N(R^{C1A})(R^{C1B}), —C(═O)OR^{C1A}, —S(O)₂R^{C1A}, —OC(═O)R^{C1A}, —OC(═O)N(R^{C1A})(R^{C1B}), —OC(═O)OR^{C1A}, —NR^{C1B}C(═O)R^{C1A}, —NR^{C1B}C(═O)N(R^{C1A})(R^{C1B}), or —NR^{C1B}C(═O)OR^{C1A} wherein R^{C1A} is unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups; and R^{C1B} is hydrogen, unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups; or R^{C1A} and R^{C1B} are joined to form an 4- to 6-membered heterocyclyl unsubstituted or substituted with 1 or 2 R^{D1} groups; and wherein each instance of R^{D1} is independently halogen, —CN, —OR^{D1A}, unsubstituted C₁₋₃alkyl, or C₁₋₃haloalkyl, wherein R^{D1A} is hydrogen, unsubstituted C₁₋₃alkyl, or C₁₋₃haloalkyl.

By way of example, in certain embodiments, substituted C₁₋₃alkyl refers to a C₁₋₃alkyl substituted with 1, 2, or 3 R^{C1} groups, as valency permits, selected from the group consisting of halogen, —CN, —OR^{C1B}, —SR^{C1B}, —N(R^{C1A})(R^{C1B}), —C(═O)R^{C1A}, —C(═O)N(R^{C1A})(R^{C1B}), —C(═O)OR^{C1A}, —S(O)₂R^{C1A}, —OC(═O)R^{C1A}, —OC(═O)N(R^{C1A})(R^{C1B}), —OC(═O)OR^{C1A}, —NR^{C1B}C(═O)R^{B1A}, —NR^{C1B}C(═O)N(R^{C1A})(R^{C1B}), and —NR^{C1B}C(═O)OR^{C1A}. In certain embodiments, any recited instance of substituted C₁₋₃alkyl refers to a C₁₋₃alkyl substituted with 1 or 2 R^{C1} groups selected from the group consisting of —CN, —OR^{C1B}, —N(R^{C1A})(R^{C1B}), —C(═O)N(R^{C1A})(R^{C1B}), and —C(═O)OR^{C1A}.

In certain embodiments, at least one instance of R^{C1} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂). Such embodiments are particularly envisioned for substitution on a C₃₋₆ carbocyclyl, 4- to 6-membered heterocyclyl, or 5- to 6-membered ring.

In certain embodiments, at least one instance of R^{C1} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). Such embodiments are also particularly envisioned for substitution on a C₃₋₆ carbocyclyl, 4- to 6-membered heterocyclyl, or 5- to 6-membered ring.

In certain embodiments, at least one instance of R^{C1} is halogen, i.e., —F, —Cl, —Br, or —I. In certain embodiments, at least one instance of R^{C1} is —F or —Cl. Such embodiments are also particularly envisioned for substitution on a C₃₋₆ carbocyclyl, 4- to 6-membered heterocyclyl, or 5- to 6-membered ring.

In certain embodiments, at least one instance of R^{C1} is —CN. For example, in certain embodiments, C₁₋₃ alkyl groups are contemplated substituted by —CN. In certain embodiments, C₂ alkyl groups are contemplated substituted by 1 —CN group, e.g., of formula:

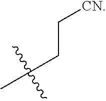 [SMILES: *CCC#N]

In certain embodiments, at least one instance of R^{C1} is —OR^{C1B}, wherein R^{C1B} is hydrogen, unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups. For example, in certain embodiments, C₁₋₃ alkyl groups are contemplated substituted by —OR^{C1B}. In certain embodiments, C₁₋₃ alkyl groups are contemplated substituted by 1 or 2 —OR^{C1B} groups, e.g., of formula:

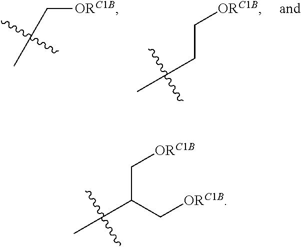

In certain embodiments, R^{C1} is —OR^{C1B}, wherein R^{C1B} is hydrogen.

In certain embodiments, R^{C1} is —OR^{C1B}, wherein R^{C1B} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂).

In certain embodiments, R^{C1} is —OR^{C1B}, wherein R^{C1B} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{C1} is —OR^{C1B}, wherein R^{C1B} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, R^{C1} is —OR^{C1B}, wherein R^{C1B} is C₃₋₆carbocylyl or 4-6 membered heterocyclyl, each independently unsubstituted or substituted with 1 or 2 R^{D} groups, wherein R^{D1} is independently halogen, —CN, —OR^{D1A}, unsubstituted C₁₋₃alkyl, or C₁₋₃haloalkyl, and wherein R^{D1A} is hydrogen, unsubstituted C₁₋₃alkyl, or C₁₋₃haloalkyl. In certain embodiments, R^{C1} is —OR^{C1B}, wherein R^{C1B} is C₃carbocylyl (e.g., cyclopropyl) unsubstituted or substituted with 1 or 2 R^{D1} groups. In certain embodiments, R^{C1} is —OR^{C1B}, wherein R^{C1B} is 4-membered heterocyclyl (e.g., azetidinyl, oxetanyl), unsubstituted or substituted with 1 or 2 R^{D1} groups. In certain embodiments, R^{C1} is —OR^{C1B}, wherein R^{C1B} is 5-membered heterocyclyl (e.g., tetrahydrofuranyl), unsubstituted or substituted with 1 or 2 R^{D1} groups. In certain embodiments, R^{C1} is —OR^{C1B}, wherein R^{C1B} is 6-membered heterocyclyl (e.g., tetrahydropyranyl), unsubstituted or substituted with 1 or 2 R^{D1} groups. In certain embodiments, such groups are unsubstituted by R^{D}1. In other embodiments, at least one instance of R^{D1} is halogen (i.e., —F, —Cl, —Br, or —I), —CN, —OR^{D1A} (e.g., —OH, —OCH₃), or unsubstituted C₁₋₃alkyl (e.g., —CH₃, —CH₂CH₃).

In certain embodiments, at least one instance of R^{C1} is —SR^{C1B}, wherein R^{C1B} is hydrogen, unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups.

In certain embodiments, R^{C1} is —SR^{C1B}, wherein R^{C1B} is hydrogen.

In certain embodiments, R^{C1} is —SR^{C1B}, wherein R^{C1B} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂).

In certain embodiments, R^{C1} is —SR^{C1B}, wherein R^{C1B} is C₁₋₃haloalkyl, e.g., CI haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{C1} is —SR^{C1B}, wherein R^{C1B} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, R^{C1} is —SR^{C1B}, wherein R^{C1B} is C₃₋₆carbocylyl or 4-6 membered heterocyclyl, each independently unsubstituted or substituted with 1 or 2 R^{D1} groups, wherein R^{D1} is independently halogen, —CN, —OR^{D1A}, unsubstituted C₁₋₃alkyl, or C₁₋₃haloalkyl, and wherein R^{D1A} is hydrogen, unsubstituted C₁₋₃alkyl, or C₁₋₃haloalkyl. In certain embodiments, R^{C1} is —SR^{C1B}, wherein R^{C1B} is C₃carbocylyl (e.g., cyclopropyl) unsubstituted or substituted with 1 or 2 R^{D1} groups. In certain embodiments, R^{C1} is —SR^{C1B}, wherein R^{C1B} is 4-membered heterocyclyl (e.g., azetidinyl, oxetanyl), unsubstituted or substituted with 1 or 2 R^{D1} groups. In certain embodiments, R^{C1} is —SR^{C1B}, wherein R^{C1B} is 5-membered heterocyclyl (e.g., tetrahydrofuranyl), unsubstituted or substituted with 1 or 2 R^{D1} groups. In certain embodiments, R^{C1} is —SR^{C1B}, wherein R^{C1B} is 6-membered heterocyclyl (e.g., tetrahydropyranyl), unsubstituted or substituted with 1 or 2 R^{D1} groups. In certain embodiments, such groups are unsubstituted by R^{D1}. In other embodiments, such groups are substituted, e.g., wherein at least one instance of R^{D}1 is halogen (i.e., —F, —Cl, —Br, or —I), —CN, —OR^{D1A} (e.g., —OH, —OCH₃), or unsubstituted C₁₋₃alkyl (e.g., —CH₃, —CH₂CH₃).

In certain embodiments, at least one instance of R^{C1} is —N(R^{C1A})(R^{C1B}) or —C(═O)N(R^{C1A})(R^{C1B}) or —OC(═O)N(R^{C1A})(R^{C1B}) or —NR^{C1B}C(═O)N(R^{C1A})(R^{C1B}), wherein R^{C1A} is unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups; R^{C1B} is hydrogen, unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups; or R^{C1A} and R^{C1B} are joined to form an 4- to 6-membered heterocyclyl unsubstituted or substituted with 1 or 2 R^{D1} groups. For example, in certain embodiments, C₁₋₃ alkyl groups are contemplated substituted by —N(R^{C1A})(R^{C1B}) or —C(═O)N(R^{C1A})(R^{C1B}). In certain embodiments, C₁₋₃ alkyl groups are contemplated substituted by 1 —N(R^{C1A})(R^{C1B}) or —C(═O)N(R^{C1A})(R^{C1B}) group, e.g., of formula:

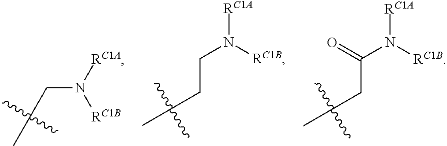

In certain embodiments of —N(R^{C1A})(R^{C1B}) or —C(═O)N(R^{C1A})(R^{C1B}) or —OC(═O)N(R^{C1A})(R^{C1B}) or —NR^{C1B}C(═O)N(R^{C1A})(R^{C1B}), R^{C1A} and R^{C1B} do not join to form a cyclic ring structure, such that R^{C1A} is unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups; and R^{C1B} is hydrogen, unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups. In certain embodiments, R^{C1B} is hydrogen or unsubstituted C₁₋₃alkyl (e.g., —CH₃). In certain embodiments, R^{C1A} is unsubstituted C₁₋₃alkyl (e.g., —CH₃), C₁₋₃haloalkyl (e.g., —CF₃, —CH₂F, —CHF₂, —CH₂Cl, —CH₂CF₃, —CH₂CHF₂, —CH(CH₃)CHF₂, or —CH(CH₃)CF₃), C₃carbocylyl (e.g., cyclopropyl) unsubstituted or substituted with 1 or 2 R^{D1} groups, 4-membered heterocyclyl (e.g., azetidinyl, oxetanyl), unsubstituted or substituted with 1 or 2 R^{D1} groups, 5-membered heterocyclyl (e.g., tetrahydrofuranyl), unsubstituted or substituted with 1 or 2 R^{D1} groups, or 6-membered heterocyclyl (e.g., tetrahydropyranyl), unsubstituted or substituted with 1 or 2 R^{D1} groups. In certain embodiments, such groups are unsubstituted by R^{D1}. In other embodiments, such groups are substituted, e.g., wherein at least one instance of R^{D1} is halogen (i.e., —F, —Cl, —Br, or —I), —CN, —OR^{D1A} (e.g., —OH, —OCH₃), or unsubstituted C₁₋₃alkyl (e.g., —CH₃, —CH₂CH₃).

In certain embodiments of —N(R^{C1A})(R^{C1B}) or —C(═O)N(R^{C1A})(R^{C1B}) or —OC(═O)N(R^{C1A})(R^{C1B}) or —NR^{C1B}C(═O)N(R^{C1A})(R^{C1B}), wherein R^{C1B} is hydrogen or —CH₃, any recited instance of —N(R^{C1A})(R^{C1B}) (e.g., for example, alone or part of a group, such as —C(═O) N(R^{C1A})(R^{C1B}) or —NR^{C1B}C(═O)N(R^{C1A})(R^{C1B}) or —OC(═O)N(R^{C1A})(R^{C1B})) independently refers to a group selected from:

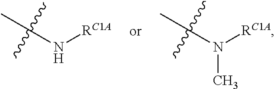

wherein R^{C1A} is as defined herein.

In certain embodiments of —N(R^{C1A})(R^{C1B}) or —C(═O)N(R^{C1A})(R^{C1B}) or —OC(═O)N(R^{C1A})(R^{C1B}) or —NR^{C1B}C(═O)N(R^{C1A})(R^{C1B}), R^{C1A} and R^{C1B} are joined to form an 4- to 6-membered heterocyclyl unsubstituted or substituted with 1 or 2 R^{D1} groups, e.g., for example, in certain embodiments, R^{C1A} and R^{C1B} are joined to form an 4-membered heterocyclyl (e.g., azetidinyl), unsubstituted or substituted with 1 or 2 R^{D1} groups, 5-membered heterocyclyl (e.g., pyrrolidinyl, pyrrolidin-2-one, oxazolidin-2-one), unsubstituted or substituted with 1 or 2 R^{D1} groups, or 6-membered heterocyclyl (e.g., morpholinyl, piperidinyl, piperazinyl), unsubstituted or substituted with 1 or 2 R^{D1} groups. In certain embodiments, such groups are unsubstituted by R^{D1}. In other embodiments, such groups are substituted, e.g., wherein at least one instance of R^{D1} is halogen (i.e., —F, —Cl, —Br, or —I), —CN, —OR^{D1A} (i.e., —OH, —OCH₃), or unsubstituted C₁₋₃alkyl (—CH₃, —CH₂CH₃).

In certain embodiments of —N(R^{C1A})(R^{C1B}) or —C(═O)N(R^{C1A})(R^{C1B}) or —OC(═O)N(R^{C1A})(R^{C1B}) or —NR^{C1B}C(═O)N(R^{C1A})(R^{C1B}), wherein R^{C1A} and R^{C1B} are joined to form an 4- to 6-membered heterocyclyl, any recited instance of —N(R^{C1A})(R^{C1B}) (e.g., for example, alone or part of a group, such as —C(═O) N(R^{C1A})(R^{C1B}) or —NR^{C} BC(═O)N(R^{C1A})(R^{C1B}) or —OC(═O)N(R^{C1A})(R^{C1B})) independently refers to a group selected from:

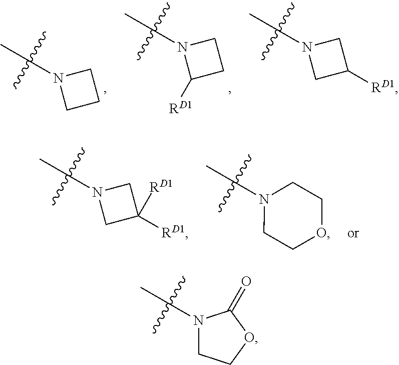

wherein R^{D1} is as defined herein.

In certain embodiments, any recited instance of —N(R^{C1A})(R^{C1B}) (e.g., for example, alone or part of a group, such as —C(═O) N(R^{C1A})(R^{C1B}) or NR^{C1B}C(═O)N(R^{C1A})(R^{C1B}) or —OC(═O)N(R^{C1A})(R^{C1B})) independently refers to:

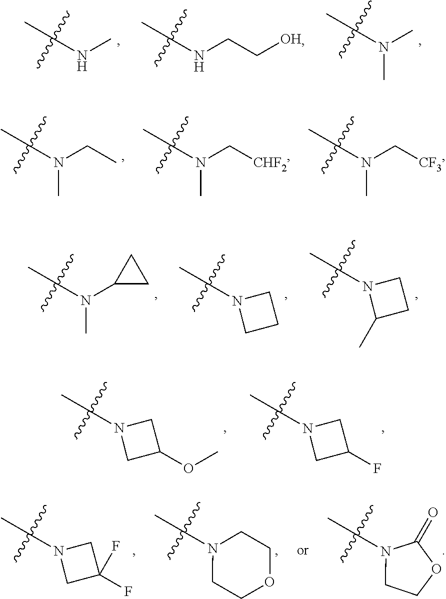

In certain embodiments, at least one instance of R^{C1} is —C(═O)R^{C1A} or —C(═O)OR^{C1A}, wherein R^{C1A} is unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups. For example, in certain embodiments, C₁₋₃ alkyl groups are contemplated substituted by —C(═O)R^{C1A} or —C(═O)OR^{C1A}. In certain embodiments, C₁₋₃ alkyl groups are contemplated substituted by one (1) —C(═O)OR^{C1A} group, e.g., of formula:

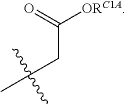

In certain embodiments, at least one instance of R^{C1} is —C(═O)R^{C1A} or —C(═O)OR^{C1A}, wherein R^{C1A} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂).

In certain embodiments, at least one instance of R^{C1} is —C(═O)R^{C1A} or —C(═O)OR^{C1A}, wherein R^{C1A} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, —C(═O)R^{C1A} or —C(═O)OR^{C1A}, wherein R^{C1A} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, at least one instance of R^{C1} is —C(═O)R^{C1A} or —C(═O)OR^{C1A}, wherein R^{C1A} is C₃₋₆carbocylyl or 4-6 membered heterocyclyl, each independently unsubstituted or substituted with 1 or 2 R^{D1} groups, wherein R^{D1} is independently halogen, —CN, —OR^{D1A}, unsubstituted C₁₋₃alkyl, or C₁₋₃haloalkyl, and wherein R^{D1A} is hydrogen, unsubstituted C₁₋₃alkyl, or C₁₋₃haloalkyl. In certain embodiments, at least one instance of R^{C1} is —C(═O)R^{C1A} or —C(═O)OR^{C1A}, wherein R^{C1A} is C₃carbocylyl (e.g., cyclopropyl) unsubstituted or substituted with 1 or 2 R^{D1} groups. In certain embodiments, at least one instance of R^{C1} is —C(═O)R^{C1A} or —C(═O)OR^{C1A}, wherein R^{C1A} is 4-membered heterocyclyl (e.g., azetidinyl, oxetanyl), unsubstituted or substituted with 1 or 2 R^{D1} groups. In certain embodiments, at least one instance of R^{C1} is —C(═O)R^{C1A} or —C(═O)OR^{C1A}, wherein R^{C1A} is 5-membered heterocyclyl (e.g., tetrahydrofuranyl), unsubstituted or substituted with 1 or 2 R^{D1} groups. In certain embodiments, at least one instance of R^{C1} is —C(═O)R^{C1A} or —C(═O)OR^{C1A}, wherein R^{C1A} is 6-membered heterocyclyl (e.g., tetrahydropyranyl), unsubstituted or substituted with 1 or 2 R^{D1} groups. In certain embodiments, such groups are unsubstituted by R^{D1}. In other embodiments, such groups are substituted, e.g., wherein at least one instance of R^{D1} is halogen (i.e., —F, —Cl, —Br, or —I), —CN, —OR^{D1A} (e.g., —OH, —OCH₃), or unsubstituted C₁₋₃alkyl (e.g., —CH₃, —CH₂CH₃).

In certain embodiments, at least one instance of R^{C1} is —OC(═O)R^{C1A} or —OC(═O)OR^{C1A}, wherein R^{C1A} is unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups.

In certain embodiments, at least one instance of R^{C1} is —OC(═O)R^{C1A} or —OC(═O)OR^{C1A}, wherein R^{C1A} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂).

In certain embodiments, at least one instance of R^{C1} is —OC(═O)R^{C1A} or —OC(═O)OR^{C1A}, wherein R^{C1A} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, at least one instance of R^{C1} is —OC(═O)R^{C1A} or —OC(═O)OR^{C1A}, wherein R^{C1A} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, at least one instance of R^{C1} is —OC(═O)R^{C1A} or —OC(═O)OR^{C1A}, wherein R^{C1A} is C₃₋₆carbocylyl or 4-6 membered heterocyclyl, each independently unsubstituted or substituted with 1 or 2 R^{D1} groups, wherein R^{D1} is independently halogen, —CN, —OR^{D1A}, unsubstituted C₁₋₃alkyl, or C₁₋₃haloalkyl, and wherein R^{D1A} is hydrogen, unsubstituted C₁₋₃alkyl, or C₁₋₃haloalkyl. In certain embodiments, at least one instance of R^{C1} is —OC(═O)R^{C1A} or —OC(═O)OR^{C1A}, wherein R^{C1A} is C₃carbocylyl (e.g., cyclopropyl) unsubstituted or substituted with 1 or 2 R^{D1} groups. In certain embodiments, at least one instance of R^{C1} is —OC(═O)R^{C1A} or —OC(═O)OR^{C1A}, wherein R^{C1A} is 4-membered heterocyclyl (e.g., azetidinyl, oxetanyl), unsubstituted or substituted with 1 or 2 R^{D1} groups. In certain embodiments, at least one instance of R^{C1} is —OC(═O)R^{C1A} or —OC(═O)OR^{C1A}, wherein R^{C1A} is 5-membered heterocyclyl (e.g., tetrahydrofuranyl), unsubstituted or substituted with 1 or 2 R^{D1} groups. In certain embodiments, at least one instance of R^{C1} is —OC(═O)R^{C1A} or —OC(═O)OR^{C1A}, wherein R^{C1A} is 6-membered heterocyclyl (e.g., tetrahydropyranyl), unsubstituted or substituted with 1 or 2 R^{D1} groups. In certain embodiments, such groups are unsubstituted by R^{D1}. In other embodiments, at least one instance of R^{D1} is halogen (i.e., —F, —Cl, —Br, or —I), —CN, —OR^{D1A} (e.g., —OH, —OCH₃), or unsubstituted C₁₋₃alkyl (e.g., —CH₃, —CH₂CH₃).

In certain embodiments, at least one instance of R^{C1} is —S(O)₂R^{C1A}, wherein R^{C1A} is unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups.

In certain embodiments, at least one instance of R^{C1} is —S(O)₂R^{C1A}, wherein R^{C1A} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂).

In certain embodiments, at least one instance of R^{C1} is —S(O)₂R^{C1A}, wherein R^{C1A} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, at least one instance of R^{C1} is —OC(═O)R^{C1A} or —OC(═O)OR^{C1A}, wherein R^{C1A} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, at least one instance of R^{C1} is —S(O)₂R^{C1A}, wherein R^{C1A} is C₃₋₆carbocylyl or 4-6 membered heterocyclyl, each independently unsubstituted or substituted with 1 or 2 R^{D1} groups, wherein R^{D1} is independently halogen, —CN, —OR^{D1A}, unsubstituted C₁₋₃alkyl, or C₁₋₃haloalkyl, and wherein R^{D1A} is hydrogen, unsubstituted C₁₋₃alkyl, or C₁₋₃haloalkyl. In certain embodiments, at least one instance of R^{C1} is —S(O)₂R^{C1A}, wherein R^{C1A} is C₃carbocylyl (e.g., cyclopropyl) unsubstituted or substituted with 1 or 2 R^{D1} groups. In certain embodiments, at least one instance of R^{C1} is —S(O)₂R^{C1A}, wherein R^{C1A} is 4-membered heterocyclyl (e.g., azetidinyl, oxetanyl), unsubstituted or substituted with 1 or 2 R^{D1} groups. In certain embodiments, at least one instance of R^{C1} is —S(O)₂R^{C1A}, wherein R^{C1A} is 5-membered heterocyclyl (e.g., tetrahydrofuranyl), unsubstituted or substituted with 1 or 2 R^{D1} groups. In certain embodiments, at least one instance of R^{C1}—S(O)₂R^{C1A}, wherein R^{C1A} is 6-membered heterocyclyl (e.g., tetrahydropyranyl), unsubstituted or substituted with 1 or 2 R^{D1} groups. In certain embodiments, such groups are unsubstituted by R^{D1}. In other embodiments, at least one instance of R^{D1} is halogen (i.e., —F, —Cl, —Br, or —I), —CN, —OR^{D1A} (e.g., —OH, —OCH₃), or unsubstituted C₁₋₃alkyl (e.g., —CH₃, —CH₂CH₃).

In certain embodiments, at least one instance of R^{C1} is —NR^{C1B}C(═O)R^{C1A} or —NR^{C1B}C(═O)OR^{C1A}, wherein R^{C1A} is unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups; R^{C1B} is hydrogen, unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups; or R^{C1A} and R^{C1B} are joined to form an 4- to 6-membered heterocyclyl unsubstituted or substituted with 1 or 2 R^{D1} groups.

In certain embodiments of —NR^{C1B}C(═O)R^{C1A} or —NR^{C1B}C(═O)OR^{C1A}, R^{C1A} and R^{C1B} do not join to form a cyclic ring structure, such that R^{C1A} is unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups, and R^{C1B} is hydrogen, unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups. In certain embodiments, R^{C1B} is hydrogen or unsubstituted C₁₋₃alkyl (e.g., —CH₃). In certain embodiments, R^{C1A} is unsubstituted C₁₋₃alkyl (e.g., —CH₃), C₁₋₃haloalkyl (e.g., —CF₃, —CH₂F, —CHF₂, —CH₂Cl, —CH₂CF₃, —CH₂CHF₂, —CH(CH₃)CHF₂, or —CH(CH₃)CF₃), C₃carbocylyl (e.g., cyclopropyl) unsubstituted or substituted with 1 or 2 R^{D1} groups, 4-membered heterocyclyl (e.g., azetidinyl, oxetanyl), unsubstituted or substituted with 1 or 2 R^{D1} groups, 5-membered heterocyclyl (e.g., tetrahydrofuranyl), unsubstituted or substituted with 1 or 2 R^{D1} groups, or 6-membered heterocyclyl (e.g., tetrahydropyranyl), unsubstituted or substituted with 1 or 2 R^{D1} groups. In certain embodiments, such groups are unsubstituted by R^{D1}. In other embodiments, at least one instance of R^{D1} is halogen (i.e., —F, —Cl, —Br, or —I), —CN, —OR^{D1A} (i.e., —OH, —OCH₃), or unsubstituted C₁₋₃alkyl (—CH₃, —CH₂CH₃).

In certain embodiments of —NR^{C1B}C(═O)R^{C1A} or —NR^{C1B}C(═O)OR^{C1A}, R^{C1A} and R^{C1B} are joined to form an 4- to 6-membered heterocyclyl unsubstituted or substituted with 1 or 2 R^{D1} groups, e.g., for example, in certain embodiments, R^{C1A} and R^{C1B} are joined to form an 4-membered heterocyclyl (e.g., azetidinyl), unsubstituted or substituted with 1 or 2 R^{D1} groups, 5-membered heterocyclyl (e.g., pyrrolidinyl, pyrrolidin-2-one, oxazolidin-2-one), unsubstituted or substituted with 1 or 2 R^{D1} groups, or 6-membered heterocyclyl (e.g., morpholinyl, piperidinyl, piperazinyl), unsubstituted or substituted with 1 or 2 R^{D1} groups. In certain embodiments, such groups are unsubstituted by R^{D1}. In other embodiments, at least one instance of R^{D1} is halogen (i.e., —F, —Cl, —Br, or —I), —CN, —OR^{D1A} (e.g., —OH, —OCH₃), or unsubstituted C₁₋₃alkyl (e.g., —CH₃, —CH₂CH₃).

In certain embodiments wherein R^{C1A} and R^{C1B} are joined to form an 4- to 6-membered heterocyclyl, any recited instance of —NR^{C1B}C(═O)OR^{C1A} independently refers to the group:

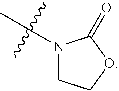 [SMILES: *N1CCOC1=O]

(IV) Ring B Substituents: Groups Comprising —N(R^{N1A})(R^{N1B}), —N(R^{B1A})(R^{B1B}), and —N(R^{B4A})(R^{B4B})

Various embodiments of Ring B substituents, e.g., wherein R^{N1} is —C(═O)N(R^{N1A})(R^{N1B}), R^{B1} is —N(R^{B1A})(R^{B1B}) (or comprises such a group, such as —C(═O)N(R^{B1A})(R^{B1B}), —OC(═O)N(R^{B1A})(R^{B1B}), or —NR^{B1B}C(═O)N(R^{B1A})(R^{B1B})), and at least one of R^{B4}, R^{B5}, R^{B6}, and R^{B7} is —N(R^{B4A})(R^{B4B}) (or comprises such a group, such as —C(═O)N(R^{B4A})(R^{B4B}), —OC(═O)N(R^{B4A})(R^{B4B}), or —NR^{B4B}C(═O)N(R^{B4A})(R^{B4B})), are contemplated herein.

For example, in each of the above-recited instances of —C(═O)N(R^{N1A})(R^{N1B}), —N(R^{B1A})(R^{B1B}), or —N(R^{B4A})(R^{B4B}), in certain embodiments the two R groups attached to the amino (N) atom do not join to form a cyclic ring structure, such that R^{N1A}, R^{B1A}, or R^{B4A} is unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups; and R^{N1B}, R^{B1B}, or R^{B4B} is hydrogen, unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups. In certain embodiments, R^{N1B}, R^{B1B}, or R^{B4B} is hydrogen or unsubstituted C₁₋₃alkyl (e.g., —CH₃). In certain embodiments, R^{N1A}, R^{B1A}, or R^{B4A} is unsubstituted C₁₋₃alkyl (e.g., —CH₃), C₁₋₃haloalkyl (e.g., —CF₃, —CH₂F, —CHF₂, —CH₂Cl, —CH₂CF₃, —CH₂CHF₂, —CH(CH₃)CHF₂, or —CH(CH₃)CF₃), C₃carbocylyl (e.g., cyclopropyl) unsubstituted or substituted with 1 or 2 R^{D1} groups, 4-membered heterocyclyl (e.g., azetidinyl, oxetanyl), unsubstituted or substituted with 1 or 2 R^{D1} groups, 5-membered heterocyclyl (e.g., tetrahydrofuranyl), unsubstituted or substituted with 1 or 2 R^{D1} groups, or 6-membered heterocyclyl (e.g., tetrahydropyranyl), unsubstituted or substituted with 1 or 2 R^{D1} groups. In certain embodiments, such groups are unsubstituted by R^{D1}. In other embodiments, at least one instance of R^{D1} is halogen (i.e., —F, —Cl, —Br, or —I), —CN, —OR^{D1A} (e.g., —OH, —OCH₃), or unsubstituted C₁₋₃alkyl (e.g., —CH₃, —CH₂CH₃).

In certain embodiments of —C(═O)N(R^{N1A})(R^{N1B}), —N(R^{B1A})(R^{B1B}), or —N(R^{B4A})(R^{B4B}), wherein R^{N1B}, R^{B1B}, Or R^{B4B} is hydrogen or —CH₃, any recited instance of —N(R^{N1A})(R^{N1B}), —N(R^{B1A})(R^{B1B}), or —N(R^{B4A})(R^{B4B}) (e.g., for example, alone or part of a group) independently refers to a group selected from:

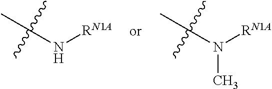

for —N(R^{N1A})(R^{N1B});

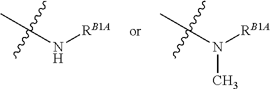

for —N(R^{B1A})(R^{B1B}); or

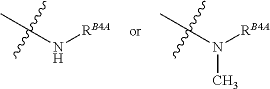

for N(R^{B4A})(R^{B4B}),
wherein R^{N1A}, R^{B1A}, and R^{B4A} are as defined herein.

In certain embodiments of —C(═O)N(R^{N1A})(R^{N1B}), —N(R^{B1A})(R^{B1B}), or —N(R^{B4A})(R^{B4B}), the two R groups attached to the amino (N) atom are joined to form an 4- to 6-membered heterocyclyl unsubstituted or substituted with 1 or 2 R^{D1} groups, e.g., for example, in certain embodiments, R^{N1A} and R^{N1B} (or R^{B1A} and R^{B1B}, or R^{B4A} and R^{B4B}) are joined to form an 4-membered heterocyclyl (e.g., azetidinyl), unsubstituted or substituted with 1 or 2 R^{D1} groups, 5-membered heterocyclyl (e.g., pyrrolidinyl, pyrrolidin-2-one, oxazolidin-2-one), unsubstituted or substituted with 1 or 2 R^{D1} groups, or 6-membered heterocyclyl (e.g., morpholinyl, piperidinyl, piperazinyl), unsubstituted or substituted with 1 or 2 R^{D1} groups. In certain embodiments, such groups are unsubstituted by R^{D1}. In other embodiments, at least one instance of R^{D1} is halogen (i.e., —F, —Cl, —Br, or —I), —CN, —OR^{D1A} (i.e., —OH, —OCH₃), or unsubstituted C₁₋₃alkyl (—CH₃, —CH₂CH₃).

In certain embodiments of —C(═O)N(R^{N1A})(R^{N1B}), —N(R^{B1A})(R^{B1B}), or —N(R^{B4A})(R^{B4B}), wherein R^{N1A} and R^{N1B}, or R^{B1A} and R^{B1B}, or R^{B4A} and R^{B4B}, are joined to form an 4- to 6-membered heterocyclyl, any recited instance of —N(R^{N1A})(R^{N1B}), —N(R^{B1A})(R^{B1B}), or —N(R^{B4A})(R^{B4B}) (e.g., for example, alone or part of a group) independently refers to a group selected from:

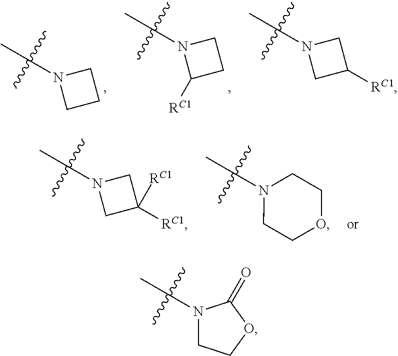

wherein R^{C1} is as defined herein.

In certain embodiments, any recited instance of —C(═O)N(R^{N1A})(R^{N1B}), —N(R^{B1A})(R^{B1B}), or —N(R^{B4A})(R^{B4B}) (e.g., for example, alone or part of a group) independently refers to:

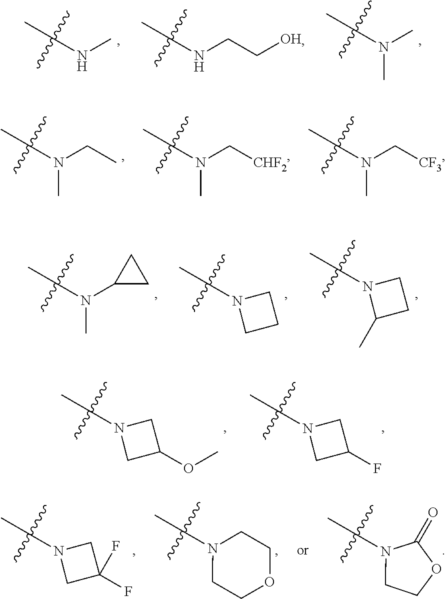

(V) Ring B Groups comprising R^{N1}, R^{B2}, and L₁

Groups R^{N1}, R^{B2} and/or L₁, are present in Ring B groups of formula (iii), (v), (vi), (vii), (viii), (ix), (x), (xii), (xiii), (xxii), (xxvii), (xxviii), and (xxiv):

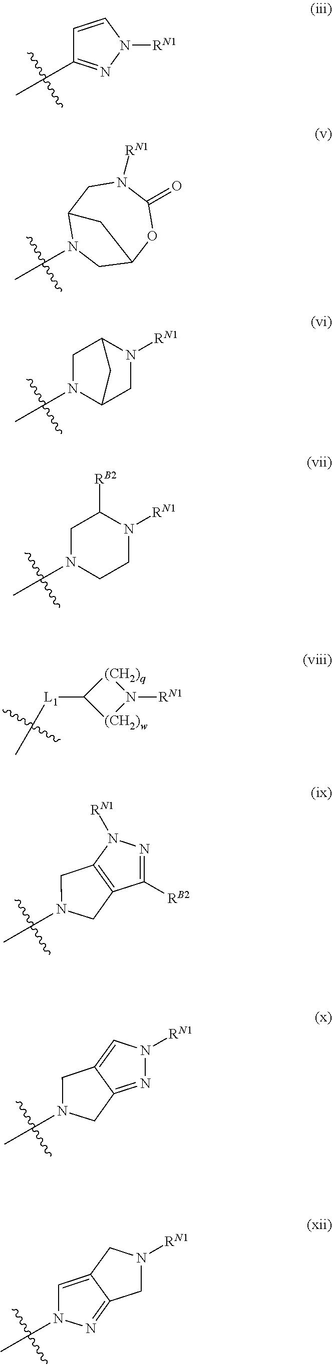

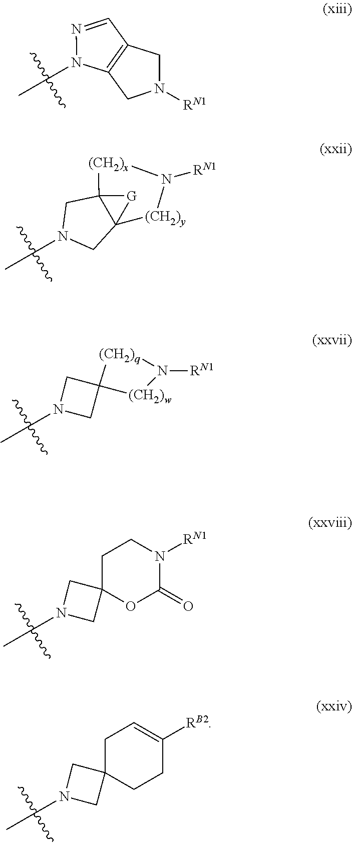

Various embodiments of R^{N1}, R^{B2} and L¹ are further contemplated herein. In particular, embodiments wherein R^{N1} is substituted or unsubstituted C₁₋₃alkyl, C₁₋₃haloalkyl substituted or unsubstituted C₃₋₆ carbocyclyl, substituted or unsubstituted 4- to 6-membered heterocyclyl, —C(═O)R^{N1A}, —C(═O)OR^{N1A}, or —S(O)₂R^{N1A}, is further contemplated herein.

Embodiments wherein R^{N1} is —C(═O)N(R^{N1A})(R^{N1B}) is contemplated in a preceding section. For example, in certain embodiments, R^{N1} is —C(═O)N(R^{N1A})(R^{N1B}), wherein R^{N1A} and R^{N1B} are as defined herein. In certain embodiments, R^{N1} is —C(═O)N(R^{N1A})(R^{N1B}), wherein the group —N(R^{N1A})(R^{N1B}) is of the formula:

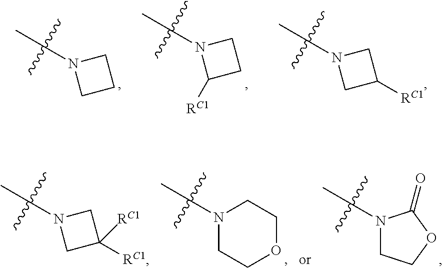

wherein R^{C1} is as defined herein.

In certain embodiments, R^{N1} is —C(═O)N(R^{N1A})(R^{N1B}), wherein the group —N(R^{N1A})(R^{N1B}) is of the formula:

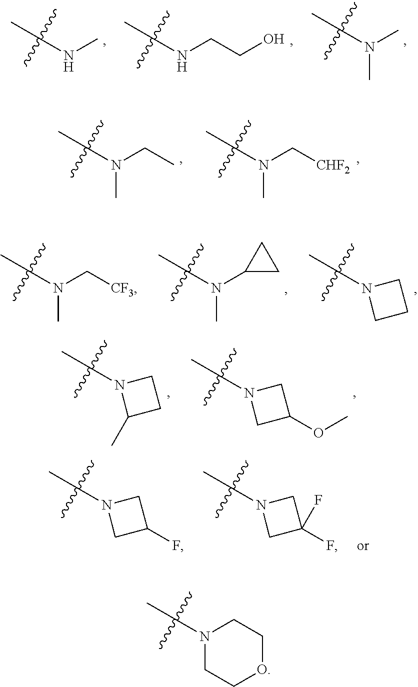

In certain embodiments, R^{N1} is substituted or unsubstituted C₁₋₃alkyl, i.e., a C₁₋₃alkyl substituted by 1, 2, or 3 R^{C1} groups as previously described herein, or an unsubstituted C₁₋₃alkyl. In certain embodiments, R^{N1} is unsubstituted C₁₋₃alkyl, i.e., C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{N1} is unsubstituted C₁₋₃alkyl of formula —CH₃, —CH₂CH₃, or —CH(CH₃)₂. In certain embodiments, R^{N1} is substituted C₁₋₃alkyl, e.g., of formula:

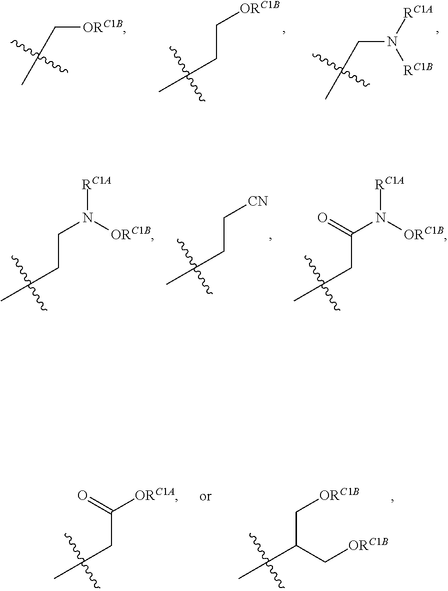

wherein R^{C1A} and R^{C1B} are as defined herein.

In certain embodiments, R^{N1} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{N1} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, R^{N1} is substituted or unsubstituted C₃₋₆carbocylyl. In certain embodiments, R^{N1} is substituted or unsubstituted C₃carbocylyl (e.g., substituted or unsubstituted cyclopropyl). In certain embodiments, such groups are unsubstituted by R^{C1}. In other embodiments, such groups are substituted, e.g., wherein at least one instance of R^{C1} is halogen (i.e., —F, —Cl, —Br, or —I), —CN, —OR^{C1A} (e.g., —OH, —OCH₃), or unsubstituted C₁₋₃alkyl (e.g., —CH₃, —CH₂CH₃).

In certain embodiments, R^{N1} is substituted or unsubstituted 4-6 membered heterocyclyl. In certain embodiments, R^{N1} is a substituted or unsubstituted 4-membered heterocyclyl (e.g., azetidinyl), substituted or unsubstituted 5-membered heterocyclyl (e.g., pyrrolidinyl, pyrrolidin-2-one, oxazolidin-2-one), or substituted or unsubstituted 6-membered heterocyclyl (e.g., morpholinyl, piperidinyl, piperazinyl). In certain embodiments, such groups are unsubstituted by R^{C1}. In other embodiments, such groups are substituted, e.g., wherein at least one instance of R^{C1} is halogen (i.e., —F, —Cl, —Br, or —I), —CN, —OR^{C1A} (e.g., —OH, —OCH₃), or unsubstituted C₁₋₃alkyl (e.g., —CH₃, —CH₂CH₃).

In certain embodiments, R^{N1} is —C(═O)R^{N1A}, —C(═O)OR^{N1A}, or —S(O)₂R^{N1A} wherein R^{N1A} is substituted or unsubstituted C₁₋₃alkyl, C₁₋₃haloalkyl, substituted or unsubstituted C₃₋₆ carbocyclyl, or substituted or unsubstituted 4- to 6-membered heterocyclyl.

In certain embodiments, R^{N1} is —C(═O)R^{NIA}, —C(═O)OR^{NIA}, or —S(O)₂R^{N1A} wherein R^{N1A} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂).

In certain embodiments, R^{N1} is —C(═O)R^{NIA}, —C(═O)OR^{NIA}, or —S(O)₂R^{N1A} wherein R^{N1A} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{N1} is —C(═O)R^{N1A}, —C(═O)OR^{N1A}, or —S(O)₂R^{N1A} wherein R^{N1A} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, R^{N1} is —C(═O)R^{NIA}, —C(═O)OR^{NIA}, or —S(O)₂R^{N1A} wherein R^{N1A} is substituted or unsubstituted C₃₋₆carbocylyl or substituted or unsubstituted 4-6 membered heterocyclyl. In certain embodiments, R^{N1} is —C(═O)R^{N1A}, —C(═O)OR^{N1A}, or —S(O)₂R^{N1A} wherein R^{N1A} is substituted or unsubstituted C₃carbocylyl (e.g., cyclopropyl). In certain embodiments, R^{N1} is —C(═O)R^{N1A}, —C(═O)OR^{N1A}, or —S(O)₂R^{N1A} wherein R^{N1A} is substituted or unsubstituted 4-membered heterocyclyl (e.g., azetidinyl, oxetanyl). In certain embodiments, R^{N1} is —C(═O)R^{N1A}, —C(═O)OR^{N1A}, or —S(O)₂R^{N1A} wherein R^{N1A} is substituted or unsubstituted 5-membered heterocyclyl (e.g., tetrahydrofuranyl). In certain embodiments, R^{N1} is —C(═O)R^{N1A}, —C(═O)OR^{N1A}, or —S(O)₂R^{N1A} wherein R^{N1A} is substituted or unsubstituted 6-membered heterocyclyl (e.g., tetrahydropyranyl). In certain embodiments, such groups are unsubstituted by R^{C1}. In other embodiments, such groups are substituted, e.g., wherein at least one instance of R^{C1} is halogen (i.e., —F, —Cl, —Br, or —I), —CN, —OR^{C1A}(e.g., —OH, —OCH₃), or unsubstituted C₁₋₃alkyl (e.g., —CH₃, —CH₂CH₃).

For example, in certain embodiments, R^{N1} is a substituted or unsubstituted 4- to 6-membered heterocyclyl comprising one oxygen ring heteroatom. In certain embodiments, R^{N1} is:

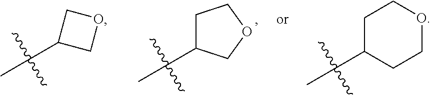 [SMILES: *C1COC1 ; *C1CCOC1 ; *C1CCOCC1]

In certain embodiments, R^{N1} is:
(a) substituted or unsubstituted C₁₋₃alkyl (—CH₃, —CH₂CH₃, —CH(CH₃)₂);
(b) C₁₋₃haloalkyl (—CF₃, —CH₂F, —CHF₂, —CH₂Cl, —CH₂CF₃, —CH₂CHF₂, —CH(CH₃)CHF₂, or —CH(CH₃)CF₃);
(c) substituted or unsubstituted C₃ carbocyclyl (e.g.,

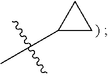 [SMILES: *C1CC1]

(d) —C(═O)R^{N1A}, —C(═O)OR^{N1A}, or —S(O)₂R^{N1A}, wherein R^{N1A} is —CH₃, CH₂CH₃, —CH(CH₃)₂, —CF₃, —CH₂F, —CHF₂, —CH₂Cl, —CH₂CF₃, —CH₂CHF₂, —CH(CH₃)CHF₂, or —CH(CH₃)CF₃;
(e) —C(═O)N(R^{N1A})(R^{N1B}), as previously contemplated, e.g., wherein N(R^{N1A})(R^{N1B}) is:

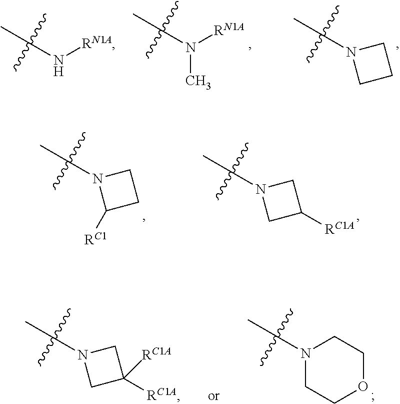

(f) substituted or unsubstituted 4-membered heterocyclyl (e.g.,

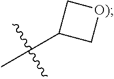

(g) substituted or unsubstituted 5-membered heterocyclyl (e.g.,

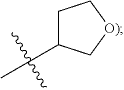

or
(h) substituted or unsubstituted 6-membered heterocyclyl (e.g.,

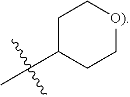

In certain embodiments, wherein Ring B is of formula:

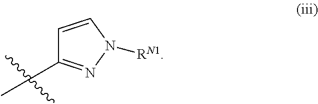

In certain embodiments of formula (iii), wherein Ring B is of formula:

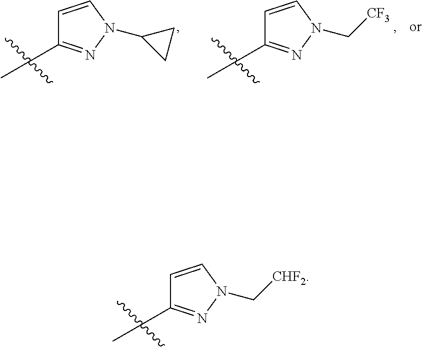 [SMILES: *c1ccn(C2CC2)n1 ; *c1ccn(CC(F)(F)F)n1 ; *c1ccn(CC(F)F)n1]

In certain embodiments, Ring B is of formula:

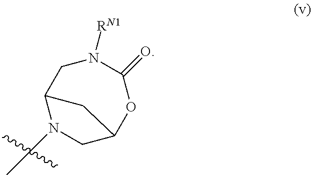

In certain embodiments of formula (v), Ring B is of formula:

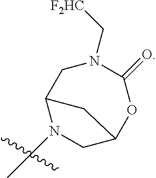 [SMILES: *N1CC2CC1CN(CC(F)F)C(=O)O2]

In certain embodiments, Ring B is of formula:

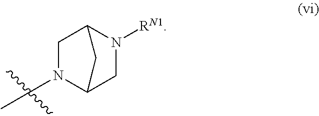

In certain embodiments of formula (vi), Ring B is of formula:

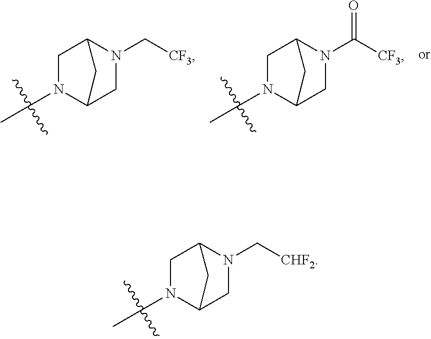 [SMILES: *N1CC2CC1CN2CC(F)(F)F ; *N1CC2CC1CN2C(=O)C(F)(F)F ; "or" ; *N1CC2CC1CN2CC(F)F]

In certain embodiments, Ring B is of formula:

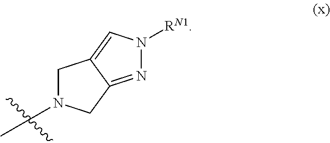

In certain embodiments of formula (x), Ring B is of formula:

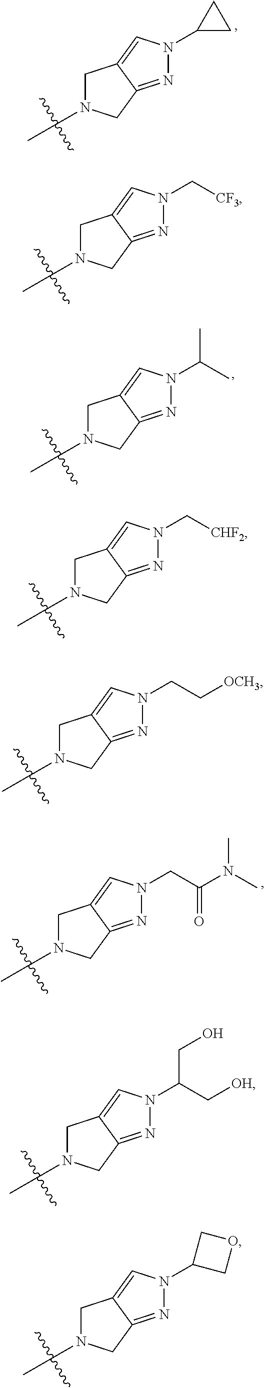

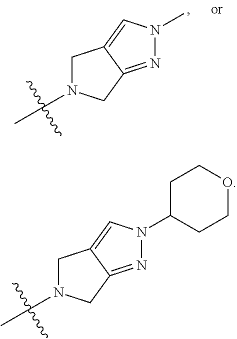

In certain embodiments, Ring B is of formula:

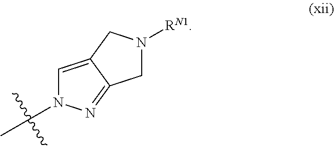

In certain embodiments of formula (xii), Ring B is of formula:

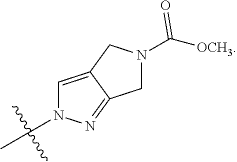 [SMILES: *n1cc2c(n1)CN(C(=O)OC)C2]

In certain embodiments, Ring B is of formula:

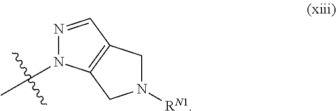

In certain embodiments of formula (xiii), Ring B is of formula:

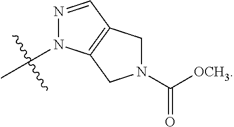 [SMILES: *n1ncc2c1CN(C(=O)OC)C2]

In certain embodiments, Ring B is of formula:

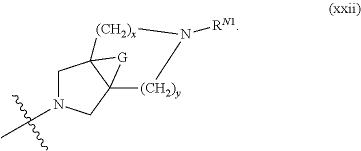

In certain embodiments of formula (xxii),

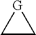

represents a single or double bond (e.g., represented by [symbol] ) to provide Ring B of formula:

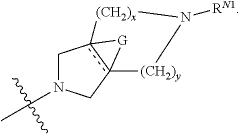

In certain embodiments of formula (xxii), [symbol] represents a single bond. In certain embodiments of formula (xxii), [symbol] represents a single bond, and the ring fusion is in the trans configuration. In certain embodiments of formula (xxii), [symbol] represents a single bond, and the ring fusion is in the cis configuration. In certain embodiments of formula (xxii), [symbol] represents a double bond.

In certain embodiments of formula (xxii), G of

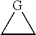

is —CH₂— to provide a cyclopropanated Ring B of formula:

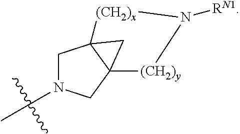

In certain embodiments of formula (xxii), x is 1 and y is 1. In certain embodiments of formula (xxii), x is 1 and y is 2. For example, in certain embodiments of formula (xxii), Ring B is of formula:

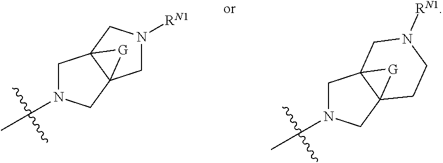

In certain embodiments of formula (xxii), Ring B is of formula:

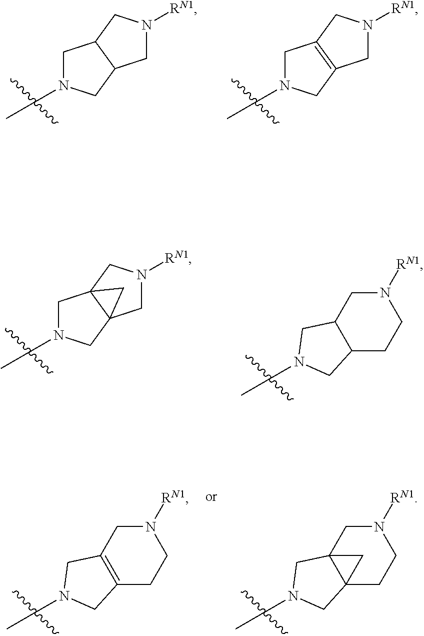

In certain embodiments of formula (xxii), Ring B is of formula:

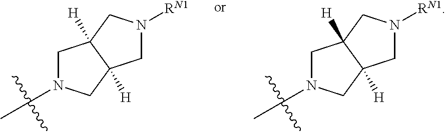

In certain embodiments of formula (xxii), Ring B is of formula:

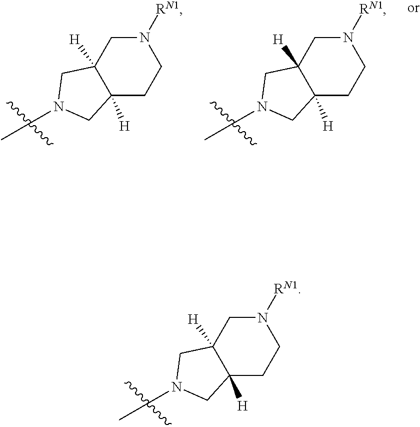

In certain embodiments of formula (xxii), Ring B is of formula:

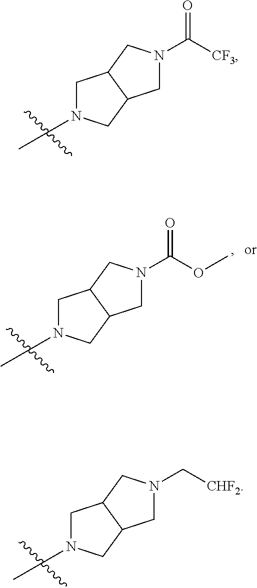 [SMILES: *N1CC2CN(C(=O)C(F)(F)F)CC2C1 ; *N1CC2CN(C(=O)OC)CC2C1 ; *N1CC2CN(CC(F)F)CC2C1]

In certain embodiments of formula (xxii), Ring B is of formula:

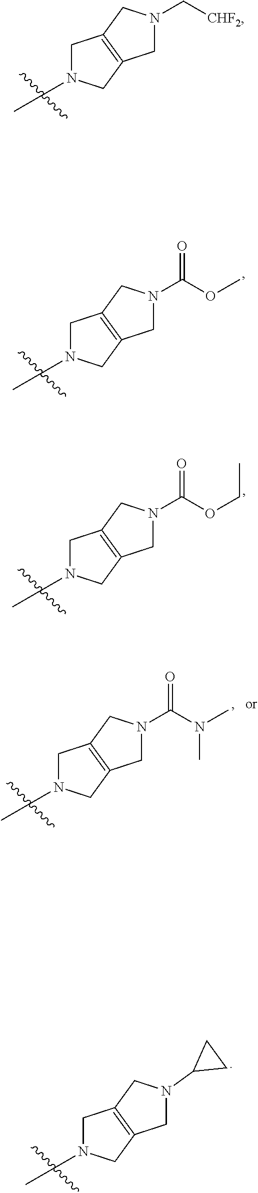 [SMILES: *N1CC2=C(C1)CN(CC(F)F)C2 ; *N1CC2=C(C1)CN(C(=O)OC)C2 ; *N1CC2=C(C1)CN(C(=O)OCC)C2 ; *N1CC2=C(C1)CN(C(=O)N(C)C)C2 ; *N1CC2=C(C1)CN(C1CC1)C2]

In certain embodiments of formula (xxii), Ring B is of formula:

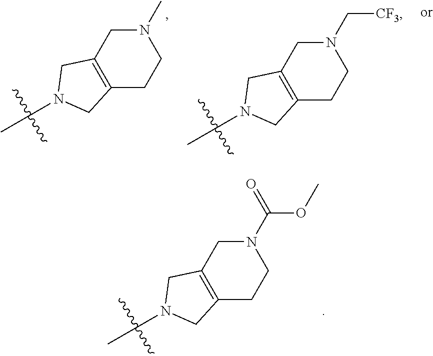 [SMILES: *N1CC2=C(C1)CN(C)CC2 ; *N1CC2=C(C1)CN(CC(F)(F)F)CC2 ; "or" ; *N1CC2=C(C1)CN(C(=O)OC)CC2]

In certain embodiments, Ring B is of formula:

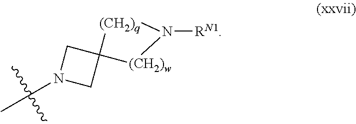

In certain embodiments of formula (xxvii), q is 1, 2, or 3 and w is 1. In certain embodiments of formula (xxvii), q is 2 and w is 0 or 2. For example, in certain embodiments of formula (xxvii), Ring B is of formula:

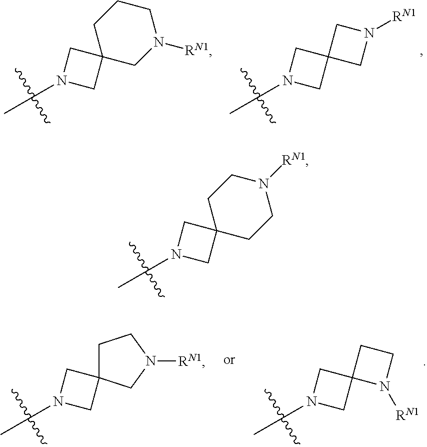

In certain embodiments of formula (xxvii), Ring B is of formula:

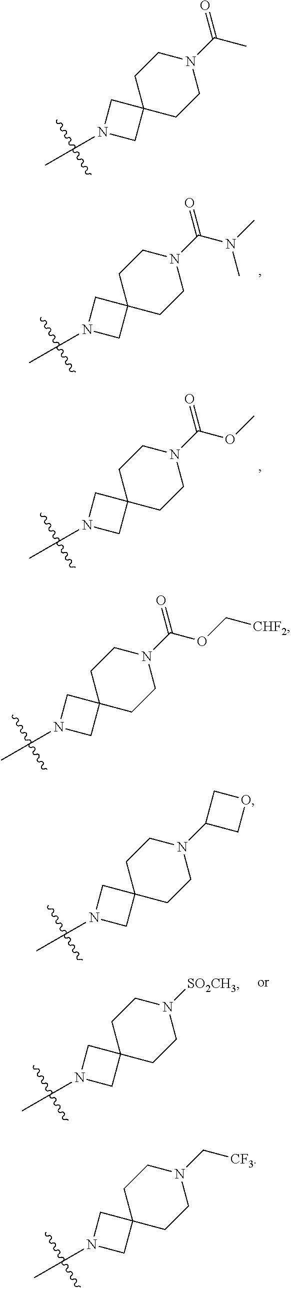

In certain embodiments of formula (xxvii), Ring B is of formula:

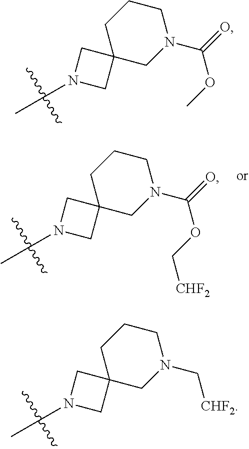 [SMILES: *N1CC2(CCCN(C(=O)OC)C2)C1 ; *N1CC2(CCCN(C(=O)OCC(F)F)C2)C1 ; "or" ; *N1CC2(CCCN(CC(F)F)C2)C1]

In certain embodiments of formula (xxvii), Ring B is of formula:

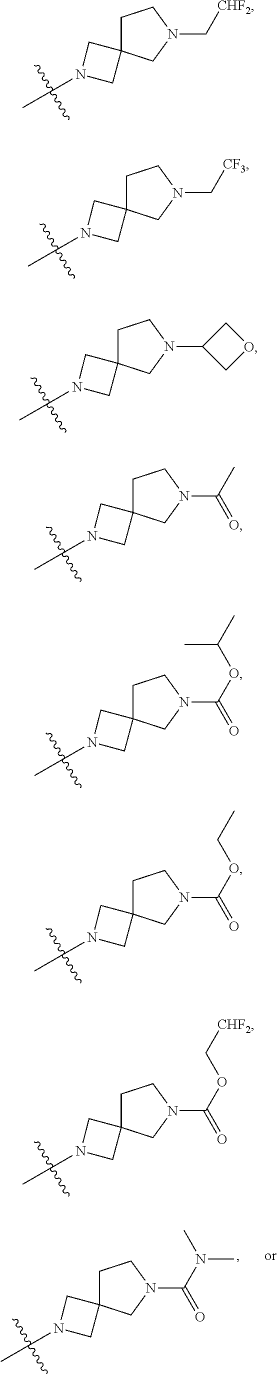

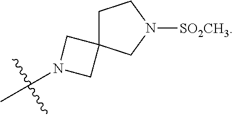 [SMILES: *N1CC2(CCN(S(C)(=O)=O)C2)C1]

In certain embodiments of formula (xxvii), Ring B is of formula:

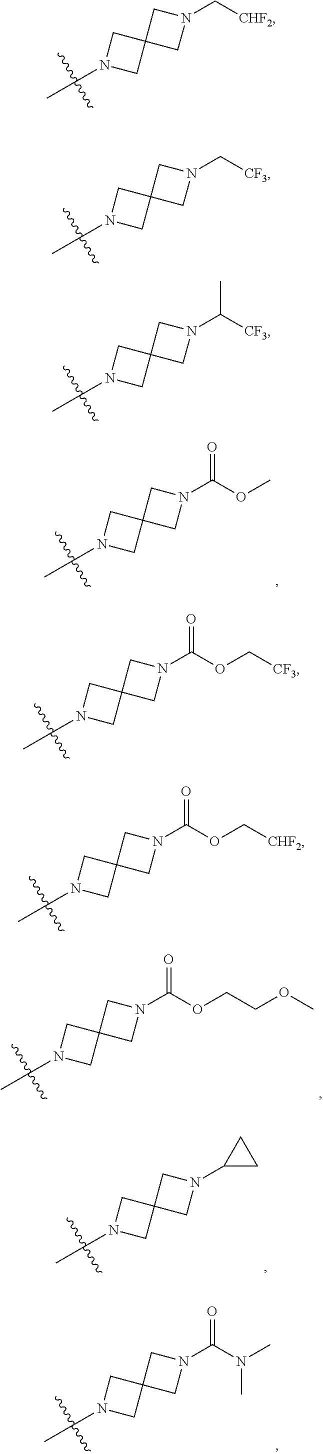

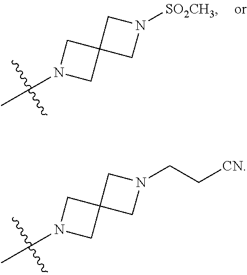 [SMILES: *N1CC2(C1)CN(S(C)(=O)=O)C2 ; *N1CC2(C1)CN(CCC#N)C2]

In certain embodiments of formula (xxvii), Ring B is of formula:

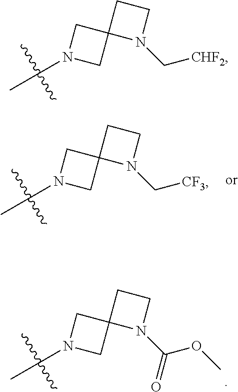 [SMILES: *N1CC2(CCN2CC(F)F)C1 ; *N1CC2(CCN2CC(F)(F)F)C1 ; "or" ; *N1CC2(CCN2C(=O)OC)C1]

In certain embodiments, Ring B is of formula:

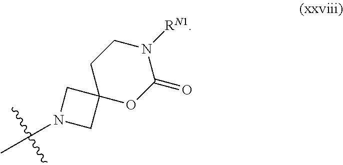

In certain embodiments of formula (xxviii), Ring B is of formula:

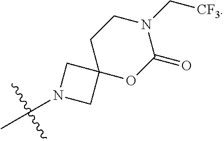 [SMILES: *N1CC2(CCN(CC(F)(F)F)C(=O)O2)C1]

In certain embodiments, Ring B is of formula:

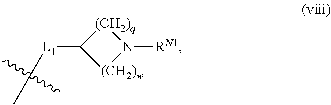

wherein L₁ is —NH—, substituted or unsubstituted C₂alkylene, substituted or unsubstituted C₂alkenylene, or substituted or unsubstituted C₂alkynylene.

In certain embodiments of formula (viii), L₁ is —NH—. In certain embodiments of formula (viii), L₁ is substituted or unsubstituted C₂alkylene. In certain embodiments of formula (viii), L₁ is substituted or unsubstituted C₂alkenylene. In certain embodiments of formula (viii), L₁ is substituted or unsubstituted C₂alkynylene. In certain embodiments of formula (viii), L₁ is an unsubstituted C₂alkylene, unsubstituted C₂alkenylene, or unsubstituted C₂alkynylene group. However, in certain embodiments of formula (viii), L₁ is a substituted C₂alkylene, substituted C₂alkenylene, or substituted C₂alkynylene group, e.g., substituted with 1 R^{C1} group such as —OR^{C1B} (e.g., —OCH₃). Exemplary substituted L¹ groups include:

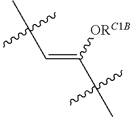

wherein R^{C1B} is as defined herein, excluding hydrogen.

Furthermore, in certain embodiments of formula (viii), q is 1, 2, or 3 and w is 1. In certain embodiments of formula (viii), q is 2 and w is 0 or 2. For example, in certain embodiments of formula (viii), Ring B is of formula:

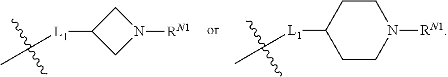

In certain embodiments of formula (viii), Ring B is of formula:

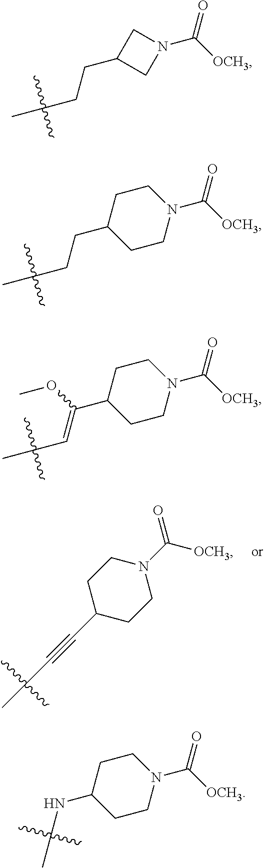 [SMILES: *CCC1CN(C(=O)OC)C1 ; *CCC1CCN(C(=O)OC)CC1 ; *C=C(OC)C1CCN(C(=O)OC)CC1 ; *C#CC1CCN(C(=O)OC)CC1 ; "or" ; *NC1CCN(C(=O)OC)CC1]

In certain embodiments, Ring B is of formula:

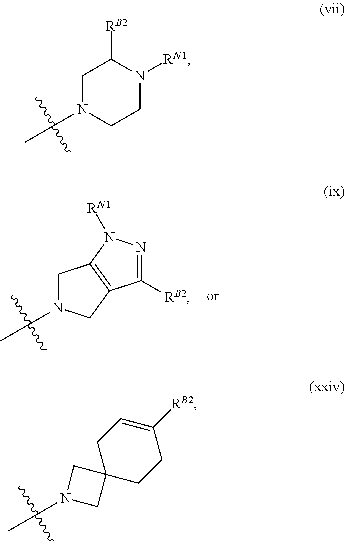

wherein R^{B2} is hydrogen, halogen, —OR^{B2A}, substituted or unsubstituted C₁₋₃alkyl, or C₁₋₃haloalkyl, and wherein R^{B2A} is substituted or unsubstituted C₁₋₃alkyl or C₁₋₃haloalkyl.

In certain embodiments, R^{B2} is hydrogen. In certain embodiments, R^{B2} is halogen, e.g., —F, —Cl, —Br, or —I. In certain embodiments, R^{B2} is substituted or unsubstituted C₁₋₃alkyl, i.e., a C₁₋₃alkyl substituted by 1, 2, or 3 R^{C1} groups as previously described herein, or an unsubstituted C₁₋₃alkyl. In certain embodiments, R^{B2} is unsubstituted C₁₋₃alkyl, i.e., C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{B2} is unsubstituted C₁₋₃alkyl of formula —CH₃, —CH₂CH₃, or —CH(CH₃)₂. In certain embodiments, R^{B2} is substituted C₁₋₃alkyl, e.g., of formula:

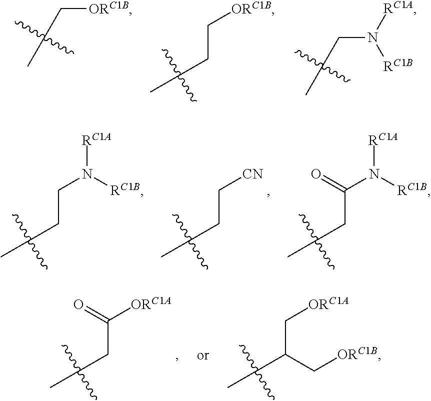

wherein R^{C1A} and R^{C1B} are as defined herein.

In certain embodiments, R^{B2} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{B2} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, R^{B2} is —OR^{B2A}, wherein R^{B2A} is substituted or unsubstituted C₁₋₃alkyl or C₁₋₃haloalkyl. In certain embodiments, R^{B2A} is substituted or unsubstituted C₁₋₃alkyl, i.e., a C₁₋₃alkyl substituted by 1, 2, or 3 R^{C1} groups as previously described herein, or an unsubstituted C₁₋₃alkyl. In certain embodiments, R^{B2A} is unsubstituted C₁₋₃alkyl, i.e., C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{B2A} is unsubstituted C₁₋₃alkyl of formula —CH₃, —CH₂CH₃, or —CH(CH₃)₂. In certain embodiments, R^{B2A} is substituted C₁₋₃alkyl, e.g., of formula:

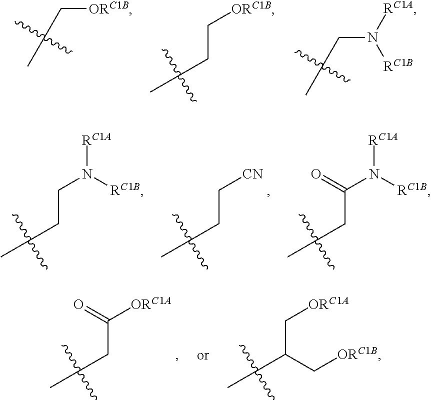

wherein R^{C1A} and R^{C1B} are as defined herein.

In certain embodiments, R^{B2} is hydrogen, —OR^{B2A}, —F, unsubstituted C₁₋₃ alkyl, C₁₋₃haloalkyl, or C₁₋₃ alkyl substituted with —OR^{C1B}.

In certain embodiments, Ring B is of formula:

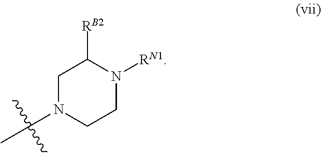

In certain embodiments of formula (vii), R^{B2} is hydrogen or —CH₃CH₃. For example, in certain embodiments of formula (vii), Ring B is of formula:

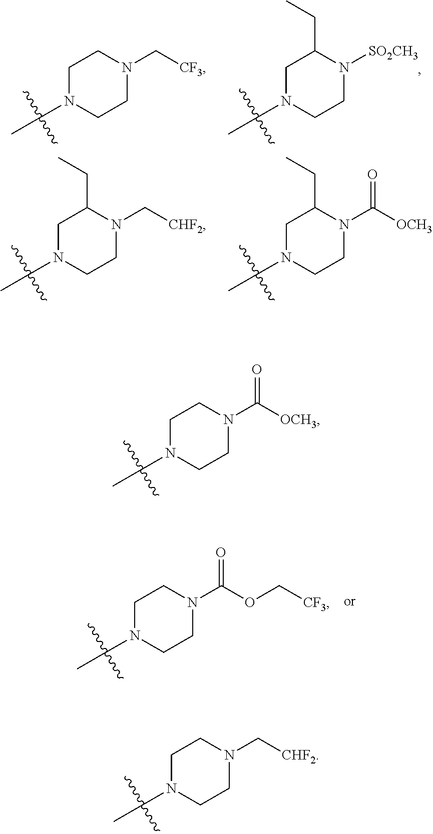

In certain embodiments, Ring B is of formula:

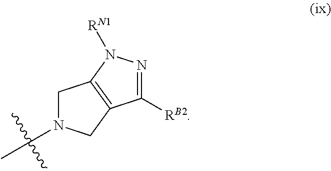

In certain embodiments of formula (ix), R^{B2} is hydrogen or —CH₃. For example, in certain embodiments of formula (ix), Ring B is of formula:

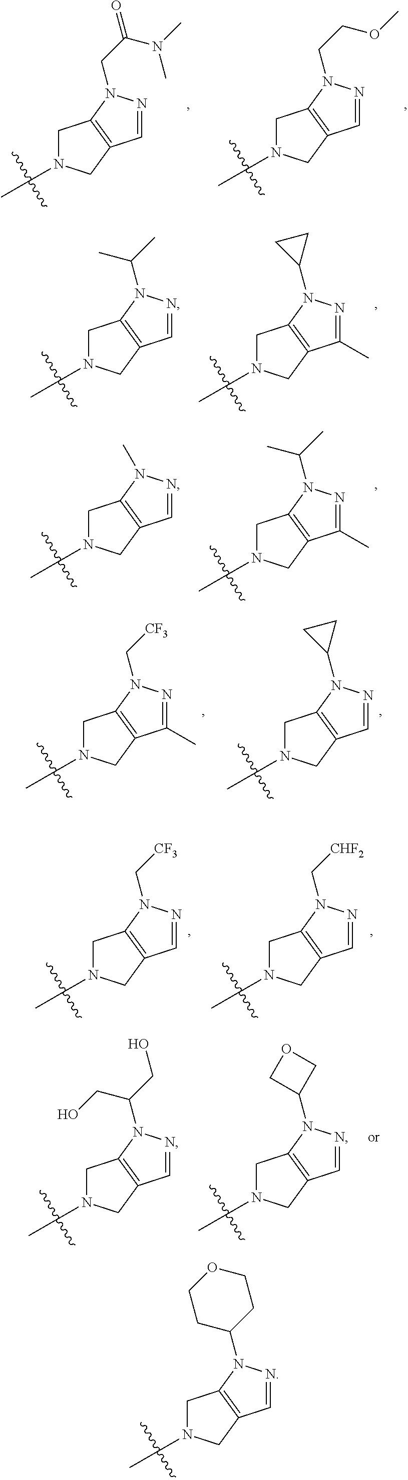

In certain embodiments, Ring B is of formula:

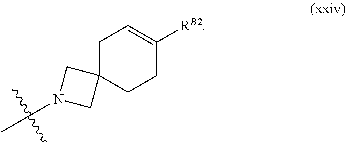

In certain embodiments of formula (xxiv), R^{B2} is hydrogen or halogen (e.g., —F, —Cl, —Br, or —I). In certain embodiments of formula (xxiv), Ring B is of formula:

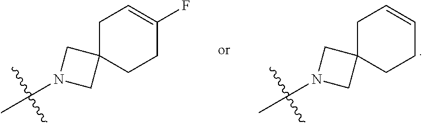 [SMILES: *N1CC2(CC=C(F)CC2)C1 ; *N1CC2(CC=CCC2)C1]

(VI) Ring B Groups Comprising R^{b1} and Optionally R^{b2}

Group R^{B1}, and optionally group R^{B2}, are present in Ring B groups of formula (i), (ii), (iv), (xi), (xxiii), and (xxvi):

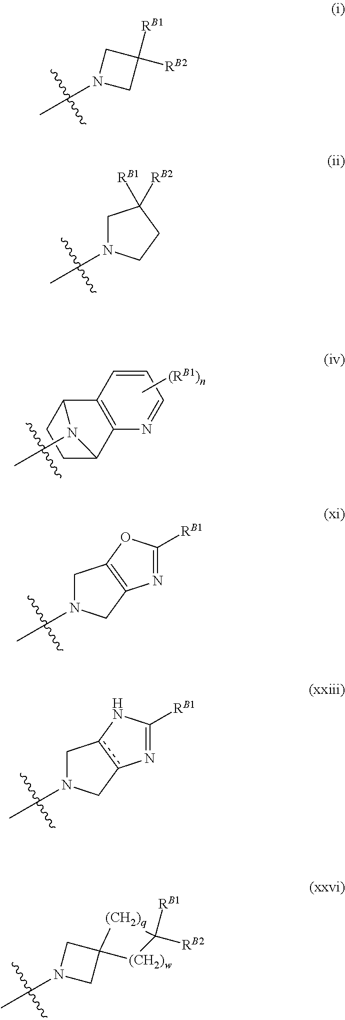

Various embodiments of R^{B1} are contemplated herein. In particular, embodiments wherein R^{B1} is substituted or unsubstituted C₁₋₃alkyl, C₁₋₃haloalkyl, halogen, —CN, —OR^{B1B}, —SR^{B1B}, substituted or unsubstituted C₃₋₆ carbocyclyl, substituted or unsubstituted 4- to 6-membered heterocyclyl, —C(═O)R^{B1A}, —C(═O)OR^{B1A}, —S(O)₂R^{B1A}, —OC(═O)R^{B1A}, —OC(═O)N(R^{B1A})(R^{B1B}), —OC(═O)OR^{B1A}, —NR^{B1B}C(═O)R^{B1A}, and —NR^{B1B}C(═O)OR^{B1A}, embodiments wherein R^{B2} is hydrogen, halogen, —OR^{B2A}, substituted or unsubstituted C₁₋₃alkyl, or C₁₋₃haloalkyl, wherein R^{B2A} is substituted or unsubstituted C₁₋₃alkyl or C₁₋₃ haloalkyl; or R^{B1} and R^{B2} are joined to form a substituted or unsubstituted 4- to 6-membered heterocyclyl, are contemplated herein.

Embodiments wherein R^{B1} is —N(R^{B1A})(R^{B1B}), —C(═O)N(R^{B1A})(R^{B1B}), —OC(═O)N(R^{B1A})(R^{B1B}), or —NR^{B1B}C(═O)N(R^{B1A})(R^{B1B}) are contemplated in a preceding section. In certain embodiments, R^{B1} is —N(R^{B1A})(R^{B1B}), wherein R^{B1A} and R^{B1B} are as defined herein. In certain embodiments, R^{B1} is —C(═O)N(R^{B1A})(R^{B1B}), wherein R^{B1A} and R^{B1B} are as defined herein. In certain embodiments, R^{B1} is —OC(═O)N(R^{B1A})(R^{B1B}), or —NR^{B1B}C(═O)N(R^{B1A})(R^{B1B}), wherein R^{B1A} and R^{B1B} are as defined herein. For example, in certain embodiments, R^{B1} is —N(R^{B1A})(R^{B1B}), —C(═O)N(R^{B1A})(R^{B1B}), —OC(═O)N(R^{B1A})(R^{B1B}), or —NR^{B1B}C(═O)N(R^{B1A})(R^{B1B}), wherein the group —N(R^{B1A})(R^{B1B}) is of the formula:

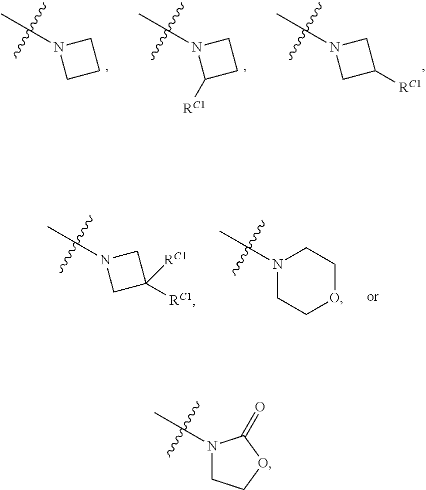

wherein R^{C1} is as defined herein.

In certain embodiments, R^{B1} is —N(R^{B1A})(R^{B1B}), —C(═O)N(R^{B1A})(R^{B1B}), —OC(═O)N(R^{B1A})(R^{B1B}), or —NR^{B1B}C(═O)N(R^{B1A})(R^{B1B}), wherein the group —N(R^{B1A})(R^{B1B}) is of the formula:

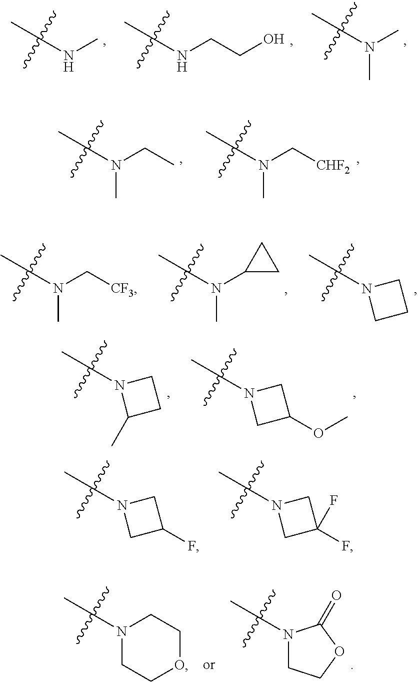

In certain embodiments, R^{B1} is halogen, i.e., —F, —Cl, —Br, or —I.

In certain embodiments, R^{B1} is —CN.

In certain embodiments, R^{B1} is —OR^{B1B} or —SR^{B1B}, wherein R^{B1B} is hydrogen, substituted or unsubstituted C₁₋₃alkyl, C₁₋₃haloalkyl, substituted or unsubstituted C₃₋₆ carbocyclyl, or substituted or unsubstituted 4- to 6-membered heterocyclyl. In certain embodiments, R^{B1} is —OR^{B1B} or —SR^{B1B}, wherein R^{B1B} is substituted or unsubstituted C₁₋₃alkyl or C₁₋₃haloalkyl. In certain embodiments, R^{B1} is —OR^{B1B} or —SR^{B1B}, wherein R^{B1B} is substituted or unsubstituted C₁₋₃alkyl, i.e., a C₁₋₃alkyl substituted by 1, 2, or 3 R^{C1} groups as previously described herein, or an unsubstituted C₁₋₃alkyl. In certain embodiments, R^{B1} is —OR^{B1B} or —SR^{B1B}, wherein R^{B1B} is unsubstituted C₁₋₃alkyl, i.e., C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{B1} is —OR^{B1B} or —SR^{B1B}, wherein R^{B1B} is unsubstituted C₁₋₃alkyl of formula —CH₃, —CH₂CH₃, or —CH(CH₃)₂. In certain embodiments, R^{B1} is —OR^{B1B} or —SR^{B1B}, wherein R^{B1B} is substituted C₁₋₃alkyl, e.g., of formula:

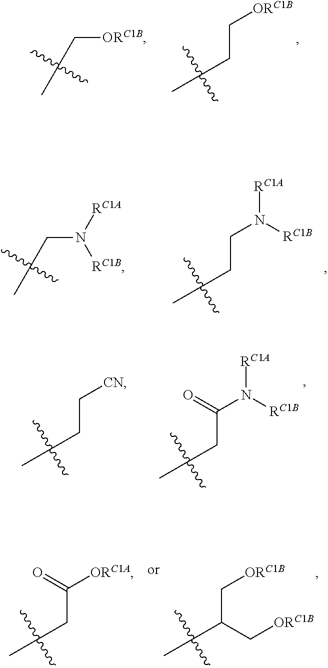

wherein R^{C1A} and R^{C1B} are as defined herein.

In certain embodiments, R^{B1} is substituted or unsubstituted C₁₋₃alkyl, i.e., a C₁₋₃alkyl substituted by 1, 2, or 3 R^{C1} groups as previously described herein, or an unsubstituted C₁₋₃alkyl. In certain embodiments, R^{B1} is unsubstituted C₁₋₃alkyl, i.e., C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{B1} is unsubstituted C₁₋₃alkyl of formula —CH₃, —CH₂CH₃, or —CH(CH₃)². In certain embodiments, R^{B1} is substituted C₁₋₃alkyl, e.g., of formula:

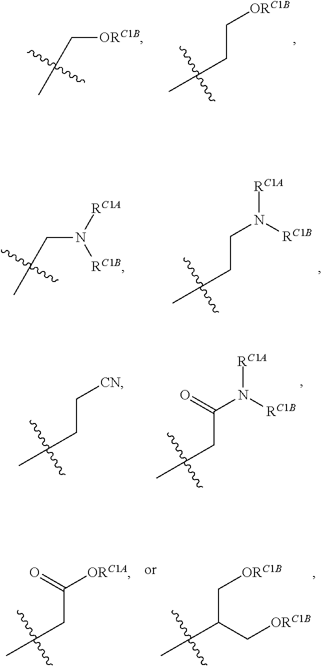

wherein R^{C1A} and R^{C1B} are as defined herein.

In certain embodiments, R^{B1} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{B1} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, R^{B1} is substituted or unsubstituted C₃₋₆carbocylyl. In certain embodiments, R^{B1} is substituted or unsubstituted C₃carbocylyl (e.g., substituted or unsubstituted cyclopropyl). In certain embodiments, such groups are unsubstituted by R^{C1}. In other embodiments, such groups are substituted, e.g., wherein at least one instance of R^{C1} is halogen (i.e., —F, —Cl, —Br, or —I), —CN, —OR^{C1A} (e.g., —OH, —OCH₃), or unsubstituted C₁₋₃alkyl (e.g., —CH₃, —CH₂CH₃).

In certain embodiments, R^{B1} is substituted or unsubstituted 4-6 membered heterocyclyl. In certain embodiments, R^{B1} is a substituted or unsubstituted 4-membered heterocyclyl (e.g., azetidinyl), substituted or unsubstituted 5-membered heterocyclyl (e.g., pyrrolidinyl, pyrrolidin-2-one, oxazolidin-2-one), or substituted or unsubstituted 6-membered heterocyclyl (e.g., morpholinyl, piperidinyl, piperazinyl). In certain embodiments, such groups are unsubstituted by R^{C1}. In other embodiments, such groups are substituted, e.g., wherein at least one instance of R^{C1} is halogen (i.e., —F, —Cl, —Br, or —I), —CN, —OR^{C1A} (e.g., —OH, —OCH₃), or unsubstituted C₁₋₃alkyl (e.g., —CH₃, —CH₂CH₃).

In certain embodiments, R^{B1} is —C(═O)R^{B1A}, —C(═O)OR^{B1A}, —OC(═O)R^{B1A}OC(═O)OR^{B1A}, —NR^{B1B}C(═O)R^{B1A}, —NR^{B1B}C(═O)OR^{B1A}, or —S(O)₂R^{B1A}, wherein R^{B1A} is substituted or unsubstituted C₁₋₃alkyl, C₁₋₃haloalkyl, substituted or unsubstituted C₃₋₆ carbocyclyl, or substituted or unsubstituted 4- to 6-membered heterocyclyl, and R^{B1B} is as defined herein.

In certain embodiments, R^{B1} is —C(═O)R^{B1A}, —C(═O)OR^{B1A}, —OC(═O)R^{B1A}OC(═O)OR^{B1A}, —NR^{B1B}C(═O)R^{B1A}, —NR^{B1B}C(═O)OR B, or —S(O)₂R^{B1A} wherein R^{B1A} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂).

In certain embodiments, R^{B1} is C(═O)R^{B1A}, —C(═O)OR^{B1A}, —OC(═O)R^{B1A}OC(═O)OR^{B1A}, —NR^{B1B}C(═O)R^{B1A}, —NR^{B1B}C(═O)OR^{B1A}, or —S(O)₂R^{B1A} wherein R^{B1A} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{B1} is C(═O)R^{B1A}, —C(═O)OR^{B1A}, —OC(═O)R^{B1A}OC(═O)OR^{B1A}, —NR^{B1B}C(═O)R^{B1A}, —NR^{B1B}C(═O)OR^{B1A}, or —S(O)₂R^{B1A} wherein R^{B1A} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, R^{B1} is C(═O)R^{B1A}, —C(═O)OR^{B1A}, —OC(═O)R^{B1A}OC(═O)OR^{B1A}, —NR^{B1B}C(═O)R^{B1A}, —NR^{B1B}C(═O)OR^{B1A}, or —S(O)₂R^{B1A} wherein R^{B1A} is substituted or unsubstituted C₃₋₆carbocylyl or substituted or unsubstituted 4-6 membered heterocyclyl. In certain embodiments, R^{B1B}C(═O)R^{B1A}, —C(═O)OR^{B1A}, —OC(═O)R^{B1A}OC(═O)OR^{B1A}, —NR^{B1B}C(═O)R^{B1A}, —NR^{B1B}C(═O)OR^{B1B}, or —S(O)₂R^{B1A} wherein R^{B1A} is substituted or unsubstituted C₃carbocylyl (e.g., cyclopropyl). In certain embodiments, R^{B1} is C(═O)R^{B1A}, —C(═O)OR^{B1A}, —OC(═O)R^{B1A}, —OC(═O)OR^{B1A}, —NR^{B1B}C(═O)R^{B1A}, —NR^{B1B}C(═O)OR^{B1A}, or —S(O)₂R^{B1A} wherein R^{B1A} is substituted or unsubstituted 4-membered heterocyclyl (e.g., azetidinyl, oxetanyl). In certain embodiments, R^{B1} is C(═O)R^{B1A}, —C(═O)OR^{B1A}, —OC(═O)R^{B1A}, —OC(═O)OR^{B1A}, —NR^{B1B}C(═O)R^{B1A}, —NR^{B1B}C(═O)OR^{B1A}, or —S(O)₂R^{B1A} wherein R^{B1A} is substituted or unsubstituted 5-membered heterocyclyl (e.g., tetrahydrofuranyl). In certain embodiments, R^{B1} is C(═O)R^{B1A}, —C(═O)OR^{B1A}, —OC(═O)R^{B1A}, —OC(═O)OR^{B1A}, —NR^{B1B}C(═O)R^{B1A}, —NR^{B1B}C(═O)OR^{B1B}, or —S(O)₂R^{B1A} wherein R^{B1A} is substituted or unsubstituted 6-membered heterocyclyl (e.g., tetrahydropyranyl). In certain embodiments, such groups are unsubstituted by R^{C1}. In other embodiments, such groups are substituted, e.g., wherein at least one instance of R^{C1} is halogen (i.e., —F, —Cl, —Br, or —I), —CN, —OR^{C1A} (e.g., —OH, —OCH₃), or unsubstituted C₁₋₃alkyl (e.g., —CH₃, —CH₂CH₃).

In certain embodiments, R^{B1} is unsubstituted C₁₋₃ alkyl, C₁₋₃haloalkyl, C₁₋₃ alkyl substituted with —OR^{C1B}, C₁₋₃ alkyl substituted with —N(R^{C1A})(R^{C1B}), C₁₋₃alkyl substituted with —CN, C₁₋₃ alkyl substituted with —C(═O)N(R^{C1A})(R^{C1B}), C₁₋₃ alkyl substituted with —C(═O)OR^{C1A}, —C(═O)N(R^{B1A})(R^{B1B}), —OC(═O)OR^{B1A}, —N(R^{B1A})(R^{B1B}), —OR^{B1B}, —SR^{B1B}, —S(O)₂R^{B1A}, —F, —Cl, —CN, substituted or unsubstituted C₃ carbocyclyl, or substituted or unsubstituted 4- to 6-membered heterocyclyl.

In certain embodiments, R^{B1} is:
(a) unsubstituted C₁₋₃alkyl (e.g., —CH₃, —CH₂CH₃, —CH₂CH₂CH₃, —CH(CH₃)₂); or
(b) C₁₋₃haloalkyl (e.g., —CF₃, —CCl₃, —CFCl₂, —CF₂Cl, —CH₂F, —CHF₂, —CH₂Cl, CHCl₂, —CF₂CF₃, —CH₂CF₃, —CH₂CHF₂, —CH₂CH₂F, —CH₂CCl₃, —CH₂CHCl₂, —CH₂CH₂Cl, —CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, —CH(CH₃)CF₃); or
(c) substituted C₁₋₃ alkyl, e.g.,

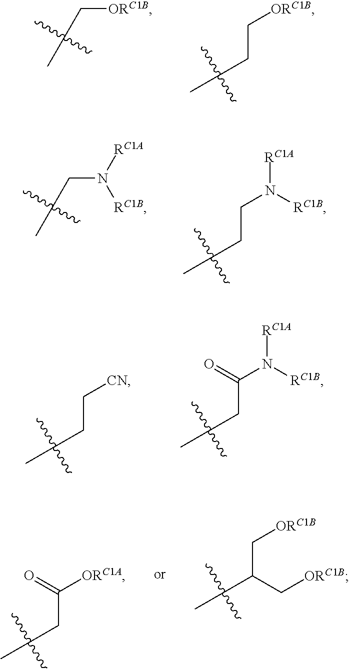

or
(d) —N(R^{B1A})(R^{B1B}), as previously contemplated, e.g.,

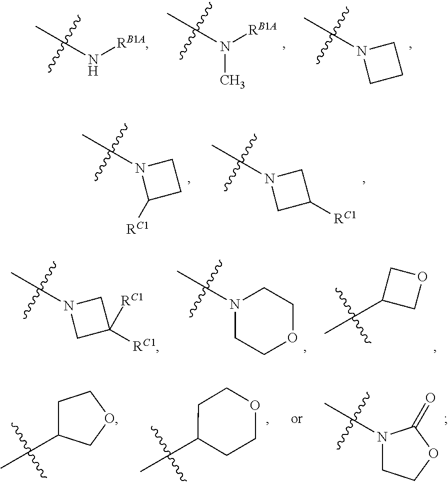

(e) substituted or unsubstituted C₃ carbocyclyl (e.g.,

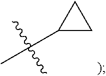 [SMILES: *C1CC1]

Furthermore, as generally defined herein, in certain embodiments, R^{B2} is hydrogen, halogen, —OR^{B2A}, substituted or unsubstituted C₁₋₃alkyl, or C₁₋₃haloalkyl, wherein R^{B2A} is substituted or unsubstituted C₁₋₃alkyl or C₁₋₃haloalkyl.

In certain embodiments, R^{B2} is hydrogen. In certain embodiments, R^{B2} is halogen, e.g., —F, —Cl, —Br, or —I. In certain embodiments, R^{B2} is substituted or unsubstituted C₁₋₃alkyl, i.e., a C₁₋₃alkyl substituted by 1, 2, or 3 R^{C1} groups as previously described herein, or an unsubstituted C₁₋₃alkyl. In certain embodiments, R^{B2} is unsubstituted C₁₋₃alkyl, i.e., C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{B2} is unsubstituted C₁₋₃alkyl of formula —CH₃, —CH₂CH₃, or —CH(CH₃)₂. In certain embodiments, R^{B2} is substituted C₁₋₃alkyl, e.g., of formula:

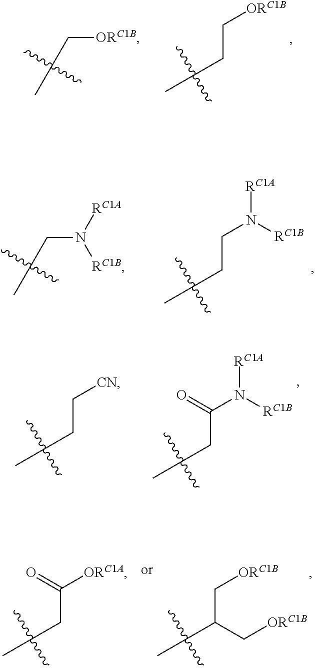

wherein R^{C1A} and R^{C1B} are as defined herein.

In certain embodiments, R^{B2} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, R^{B2} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, R^{B2} is —OR^{B2A}, wherein R^{B2A} is substituted or unsubstituted C₁₋₃alkyl or C₁₋₃haloalkyl. In certain embodiments, R^{B2A} is substituted or unsubstituted C₁₋₃alkyl, i.e., a C₁₋₃alkyl substituted by 1, 2, or 3 R^{C1} groups as previously described herein, or an unsubstituted C₁₋₃alkyl. In certain embodiments, R^{B2A} is unsubstituted C₁₋₃alkyl, i.e., C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, R^{B2A} is unsubstituted C₁₋₃alkyl of formula —CH₃, —CH₂CH₃, or —CH(CH₃)₂. In certain embodiments, R^{B2A} is substituted C₁₋₃alkyl, e.g., of formula:

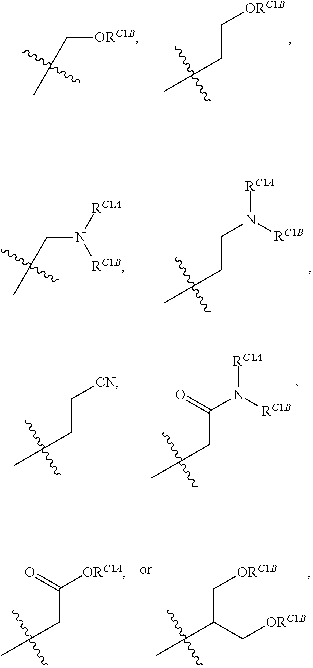

wherein R^{C1A} and R^{C1B} are as defined herein.

In certain embodiments, R^{B2} is hydrogen, —OR^{B2A}, —F, unsubstituted C₁₋₃ alkyl, C₁₋₃haloalkyl, or C₁₋₃ alkyl substituted with —OR^{C1B}.

In certain embodiments, wherein R^{B1} and R^{B2} are each present on Ring B, such as Ring B of formula (i), (ii), or (xxvi), various combinations of R^{B1} and R^{B2} are contemplated herein. For example, in certain embodiments, the following R^{B1} and R^{B2} combinations are specifically contemplated:
a. R^{B1} is —N(R^{B1A})(R^{B1B}), —OR^{B1B}, —SR^{B1B}, —S(O)₂R^{B1A}, —F, —Cl, —CN, —OC(═O)OR^{B1A}, —C(═O)N(R^{B1A})(R^{B1B}), and R^{B2} is hydrogen; or
b. R^{B1} is —F and R^{B2} is —F; or
c. R^{B1} is —OR^{B1B}, —C(═O)N(R^{B1A})(R^{B1B}), —CN, or C₁₋₃ alkyl substituted with —OR^{C1B}, C₁₋₃ alkyl substituted with —N(R^{C1A})(R^{C1B}), and R^{B2} is substituted or unsubstituted C₁₋₃ alkyl or C₁₋₃haloalkyl; or
d. R^{B1} is —OR^{B1B} and R^{B2} is —OR^{B2A}, and each instance of R^{B1B} and R^{B2A} is independently substituted or unsubstituted C₁₋₃ alkyl or C₁₋₃haloalkyl.

Furthermore, as generally defined herein, in certain embodiments, R^{B1} and R^{B2} are joined to form a substituted or unsubstituted 4- to 6-membered heterocyclyl, e.g., a substituted or unsubstituted 4-membered heterocyclyl, a substituted or unsubstituted 5-membered heterocyclyl, or a substituted or unsubstituted 6-membered heterocyclyl. For example, in certain embodiments, wherein R^{B1} is —OR^{B1B} and R^{B2} is —OR^{B2A}, R^{B1B} and R^{B1B} is —OR^{B2A} are joined to form a substituted or unsubstituted 5-membered heterocyclyl (e.g., dioxolanyl) or substituted or unsubstituted 6-membered heterocyclyl (e.g., dioxanyl).

In certain embodiments, Ring B is of formula:

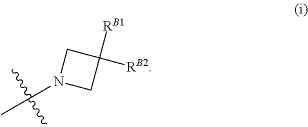

In certain embodiments of formula (i), Ring B is of formula:

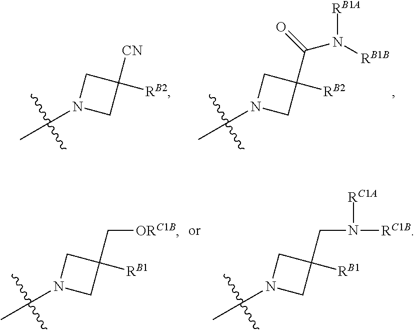

In certain embodiments of formula (i), Ring B is of formula:

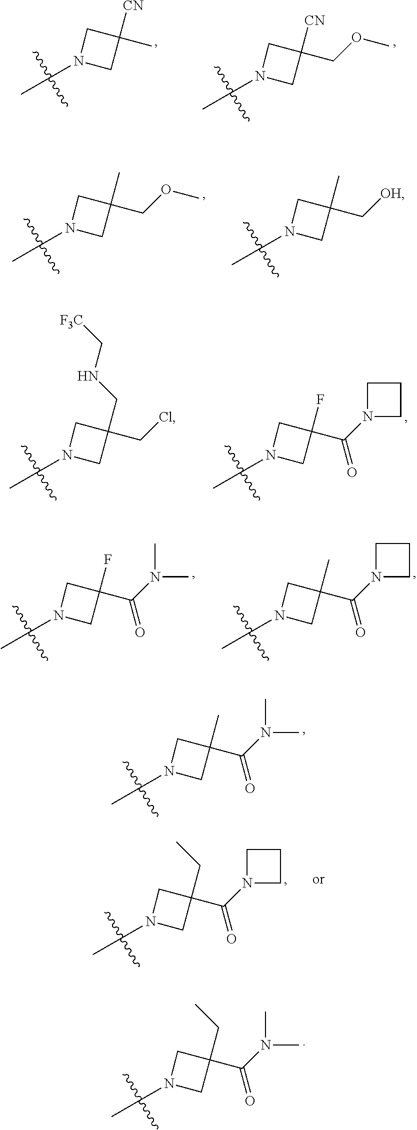

In certain embodiments, Ring B is of formula:

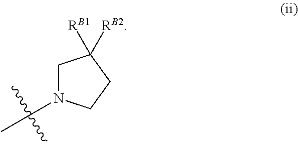

In certain embodiments of formula (ii), Ring B is of formula:

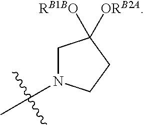

In certain embodiments of formula (ii), Ring B is of formula:

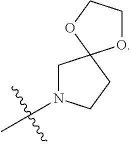 [SMILES: *N1CCC2(C1)OCCO2]

In certain embodiments, Ring B is of formula:

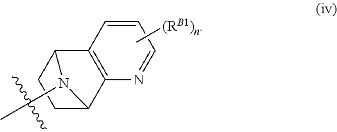

In certain embodiments of formula (iv), Ring B is of formula:

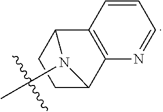 [SMILES: *N1C2CCC1c1ncccc12]

In certain embodiments, Ring B is of formula:

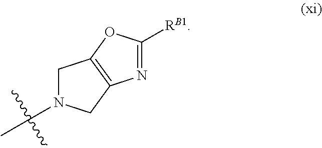

In certain embodiments of formula (xi), Ring B is of formula:

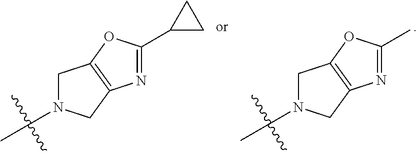

In certain embodiments, Ring B is of formula:

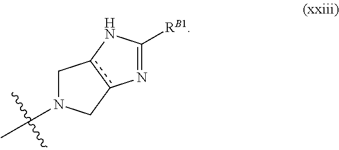

In certain embodiments of formula (xxiii), [symbol] represents a single bond. In certain embodiments of formula (xxiii), [symbol] represents a single bond, and the ring fusion is in the trans configuration. In certain embodiments of formula (xxiii), [symbol] represents a single bond, and the ring fusion is in the cis configuration. In certain embodiments of formula (xxiii), [symbol] represents a double bond.

In certain embodiments, Ring B is of formula:

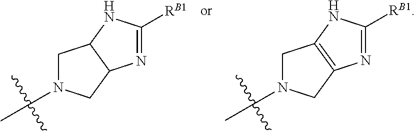

In certain embodiments, Ring B is of formula:

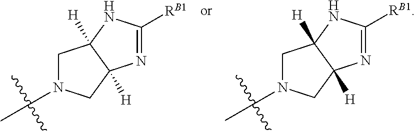

In certain embodiments, Ring B is of formula:

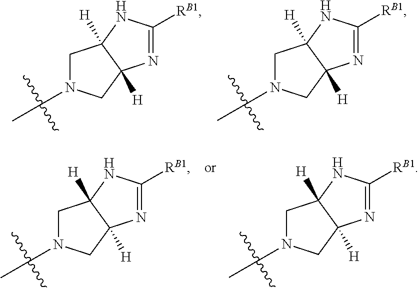

In certain embodiments of formula (xxiii), Ring B is of formula:

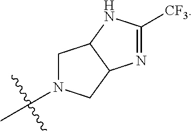 [SMILES: *N1CC2N=C(C(F)(F)F)NC2C1]

In certain embodiments, Ring B is of formula:

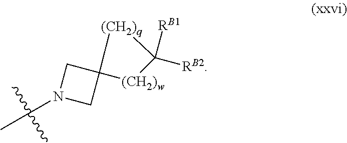

In certain embodiments of formula (xxvii), q is 1, 2, or 3 and w is 1. In certain embodiments of formula (xxvii), q is 2 and w is 0 or 2. For example, in certain embodiments of formula (xxvii), Ring B is of formula:

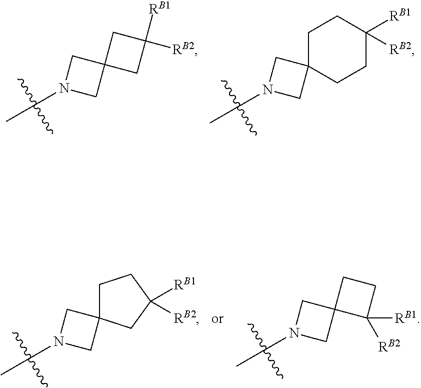

In certain embodiments of formula (xxvii), Ring B is of formula:

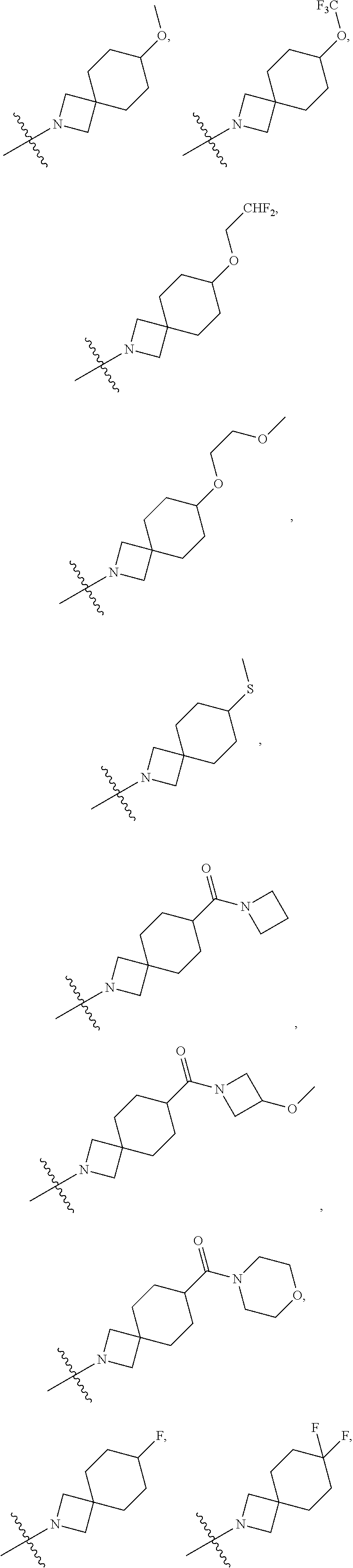

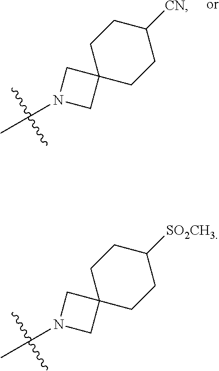 [SMILES: *N1CC2(CCC(C#N)CC2)C1 ; "or" ; *N1CC2(CCC(S(C)(=O)=O)CC2)C1]

In certain embodiments of formula (xxvii), Ring B is of formula:

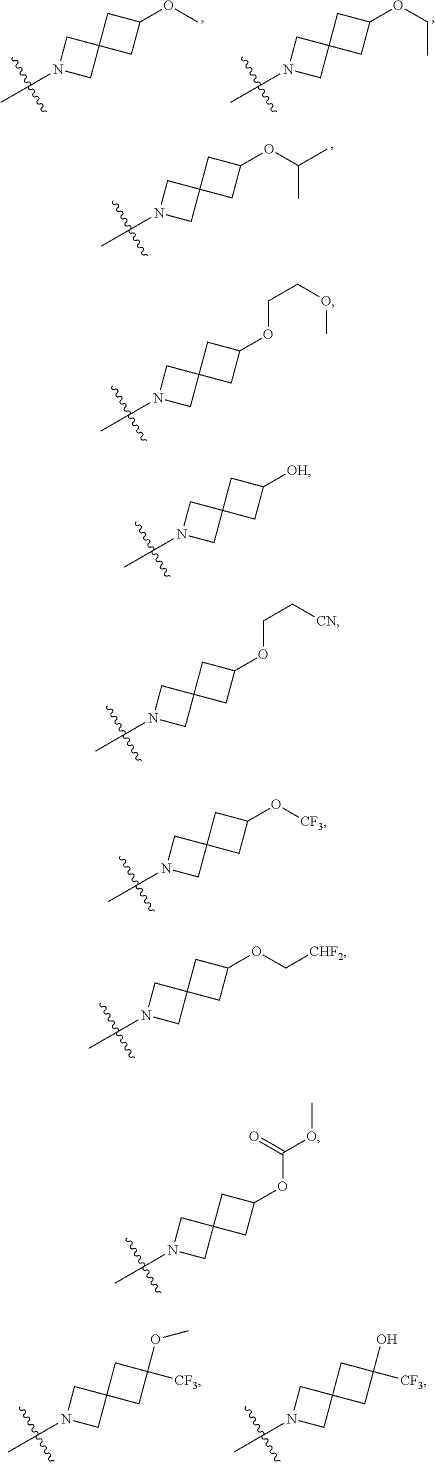

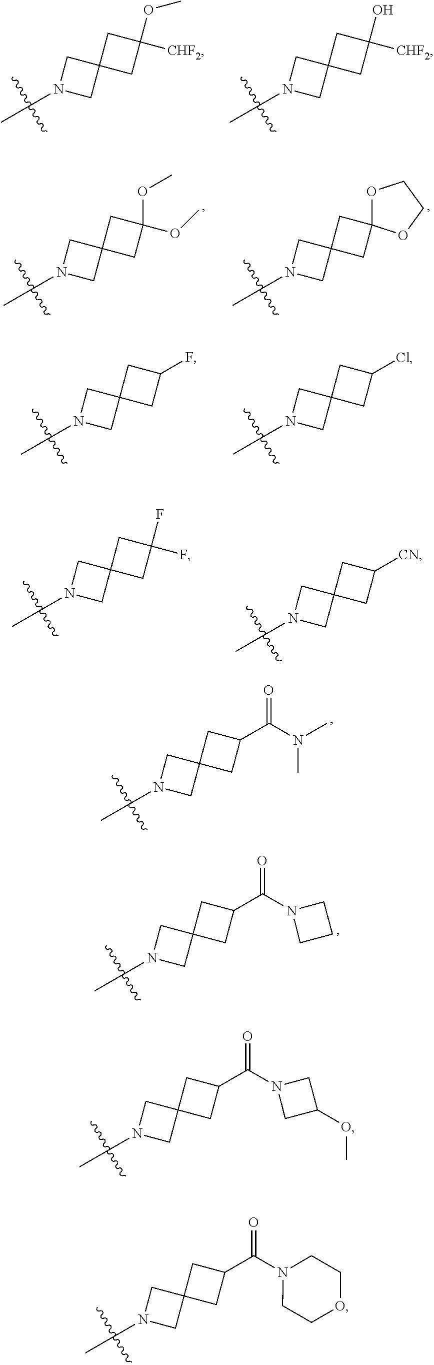

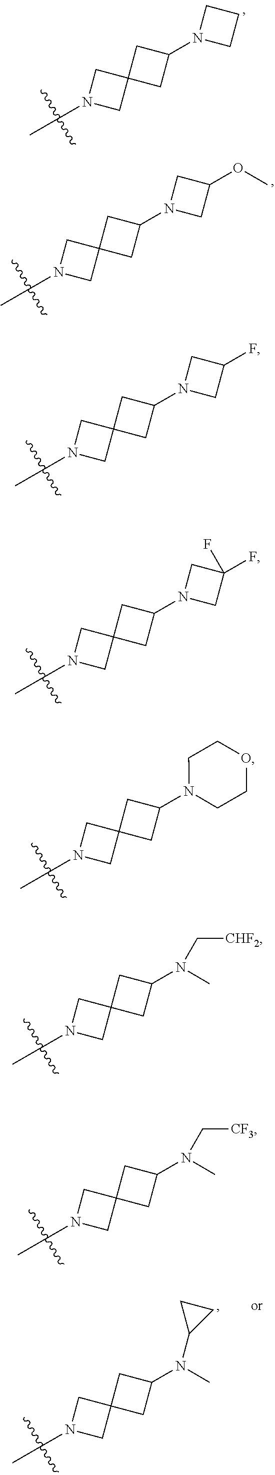

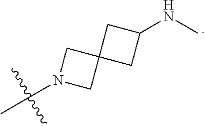 [SMILES: *N1CC2(CC(NC)C2)C1]

In certain embodiments of formula (xxvii), Ring B is of formula:

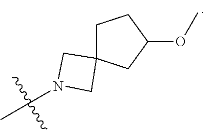 [SMILES: *N1CC2(CCC(OC)C2)C1]

In certain embodiments of formula (xxvii), Ring B is of formula:

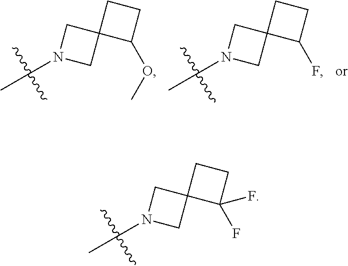

(VII) Ring B: Groups Comprising R^{N2}, R^{B3}, R^{B4}, R^{B5}, R^{B6}, and/or R^{B7}

Groups R^{N2}, R^{B3}, R^{B4}, R^{B5}, R^{B6}, and/or R^{B7} are provided in Ring B groups of formula (xiv), (xv), (xvi), (xvii), (xviii), (xix), (xx), and (xxi):

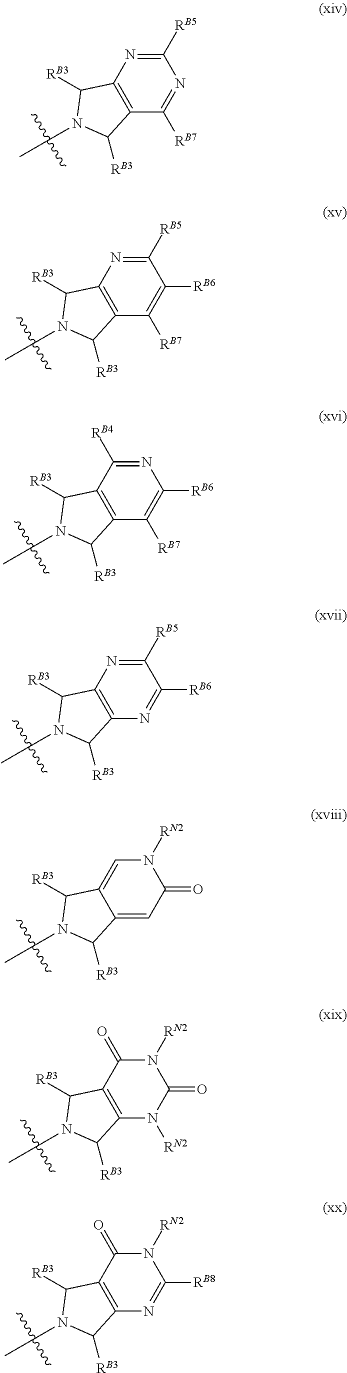

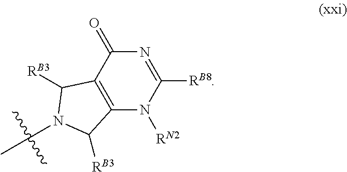

Various embodiments of R^{B4}, R^{B5}, R^{B6}, and R^{B7} are contemplated herein. In particular, embodiments wherein at least one of R^{B4}, R^{B5}, R^{B6}, and R^{B7} is hydrogen, substituted or unsubstituted C₁₋₃alkyl, C₁₋₃haloalkyl, halogen, —CN, —OR^{B4B}, —SR^{B4B} substituted or unsubstituted C₃₋₆ carbocyclyl, substituted or unsubstituted 4- to 6-membered heterocyclyl, —C(═O)R^{B4A}, —C(═O)OR^{B4A}, —S(O)₂R^{B4A}, —OC(═O)R^{B4A}OC(═O)N(R^{B4A})(R^{B4B}), —OC(═O)OR^{B4A}, —NR^{B4B}C(═O)R^{B4A}, and —NR^{B4B}C(═O)OR^{B4A}.

Embodiments wherein R^{B4}, R^{B5}, R^{B6}, or R^{B7} is —N(R^{B4A})(R^{B4B}), —C(═O)N(R^{B4A})(R^{B4B}), —OC(═O)N(R^{B4A})(R^{B4B}), or —NR^{B4B}C(═O)N(R^{B4A})(R^{B4B}) are contemplated in a preceding section. In certain embodiments, at least one of R^{B4}, R^{B5}, R^{B6}, and R^{B7} is —N(R^{B4A})(R^{B4B}), wherein R^{B4A} and R^{B4B} are as defined herein. In certain embodiments, at least one of R^{B4}, R^{B5}, R^{B6}, and R^{B7} is —C(═O)N(R^{B4A})(R^{B4B}), wherein R^{B4A} and R^{B4B} are as defined herein. In certain embodiments, at least one of R^{B4}, R^{B5}, R^{B6}, and R^{B7} is —OC(═O)N(R^{B4A})(R^{B4B}), or —NR^{B4B}C(═O)N(R^{B4A})(R^{B4B}), wherein R^{B4A} and R^{B4B} are as defined herein. For example, in certain embodiments, at least one of R^{B4}, R^{B5}, R^{B6}, and R^{B7} is —N(R^{B4A})(R^{B4B}), —C(═O)N(R^{B4A})(R^{B4B}), —OC(═O)N(R^{B4A})(R^{B4B}), or —NR^{B4B}C(═O)N(R^{B4A})(R^{B4B}), wherein the group —N(R^{B4A})(R^{B4B}) is of the formula:

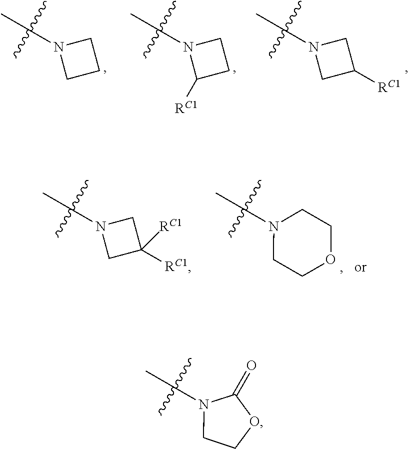

wherein R^{C1} is as defined herein.

In certain embodiments, at least one of R^{B4}, R^{B5}, R^{B6}, and R^{B7} is —N(R^{B4A})(R^{B4B}), —C(═O)N(R^{B4A})(R^{B4B}), —OC(═O)N(R^{B4A})(R^{B4B}), or —NR^{B4B}C(═O)N(R^{B4A})(R^{B4B}), wherein the group —N(R^{B4A})(R^{B4B}) is of the formula:

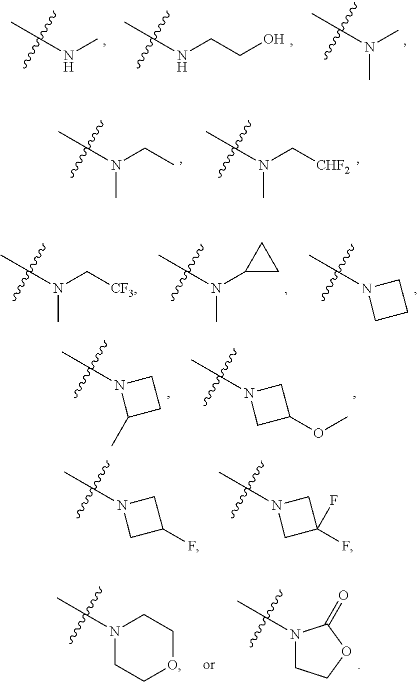

In certain embodiments, at least one of R^{B4}, R^{B5}, R^{B6}, and R^{B7} is hydrogen. In certain embodiments, two of R^{B4}, R^{B5}, R^{B6}, and R^{B7} are hydrogen. In certain embodiments, each of R^{B4}, R^{B5}, R^{B6}, and R^{B7} is hydrogen.

In certain embodiments, at least one of R^{B4}, R^{B5}, R^{B6}, and R^{B7} is halogen, i.e., —F, —Cl, —Br, or —I. In certain embodiments, at least one of R^{B4}, R^{B5}, R^{B6}, and R^{B7} is —CN.

In certain embodiments, at least one of R^{B4}, R^{B5}, R^{B6}, and R^{B7} is —OR^{B4B} or —SR^{B4B}, wherein R^{B4B} is hydrogen, substituted or unsubstituted C₁₋₃alkyl, C₁₋₃haloalkyl, substituted or unsubstituted C₃₋₆ carbocyclyl, or substituted or unsubstituted 4- to 6-membered heterocyclyl. In certain embodiments, at least one of R^{B4}, R^{B5}, R^{B6}, and R^{B7} is —OR^{B4B} or —SR^{B4B}, wherein R^{B4B} is substituted or unsubstituted C₁₋₃alkyl or C₁₋₃haloalkyl In certain embodiments, at least one of R^{B4}, R^{B5}, R^{B6}, and R^{B7} is —OR^{B4B} or —SR^{B4B}, wherein R^{B4B} is substituted or unsubstituted C₁₋₃alkyl, i.e., a C₁₋₃alkyl substituted by 1, 2, or 3 R^{C1} groups as previously described herein, or an unsubstituted C₁₋₃alkyl. In certain embodiments, at least one of R^{B4}, R^{B5}, R^{B6}, and R^{B7} is —OR^{B4B} or —SR^{B4B}, wherein R^{B4B} is unsubstituted C₁₋₃alkyl, i.e., C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, at least one of R^{B4}, R^{B5}, R^{B6}, and R^{B7} is —OR^{B4B} or —SR^{B4B}, wherein R^{B4B} is unsubstituted C₁₋₃alkyl of formula —CH₃, —CH₂CH₃, or —CH(CH₃)₂. In certain embodiments, at least one of R^{B4}, R^{B5}, R^{B6}, and R^{B7} is —OR^{B4B} or —SR^{B4B}, wherein R^{B4B} is substituted C₁₋₃alkyl, e.g., of formula:

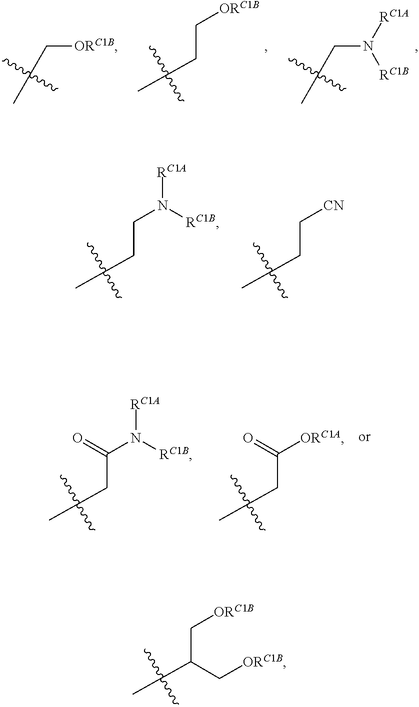

wherein R^{C1A} and R^{C1B} are as defined herein.

In certain embodiments, at least one of R^{B4}, R^{B5}, R^{B6}, and R^{B7} is substituted or unsubstituted C₁₋₃alkyl, i.e., a C₁₋₃alkyl substituted by 1, 2, or 3 R^{C1} groups as previously described herein, or an unsubstituted C₁₋₃alkyl. In certain embodiments, at least one of R^{B4}, R^{B5}, R^{B6}, and R^{B7} is unsubstituted C₁₋₃alkyl, i.e., C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, at least one of R^{B4}, R^{B5}, R^{B6}, and R^{B7} is unsubstituted C₁₋₃alkyl of formula —CH₃, —CH₂CH₃, or —CH(CH₃)₂. In certain embodiments, at least one of R^{B4}, R^{B5}, R^{B6}, and R^{B7} is substituted C₁₋₃alkyl, e.g., of formula:

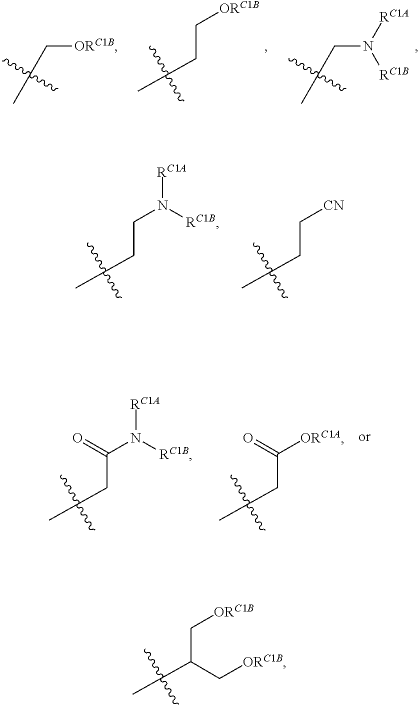

wherein R^{C1A} and R^{C1B} are as defined herein.

In certain embodiments, at least one of R^{B4}, R^{B5}, R^{B6}, and R^{B7} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, at least one of R^{B4}, R^{B5}, R^{B6}, and R^{B7} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, at least one of R^{B4}, R^{B5}, R^{B6}, and R^{B7} is substituted or unsubstituted C₃₋₆carbocylyl. In certain embodiments, at least one of R^{B4}, R^{B5}, R^{B6}, and R^{B7} is substituted or unsubstituted C₃carbocylyl (e.g., substituted or unsubstituted cyclopropyl). In certain embodiments, such groups are unsubstituted by R^{C1}. In other embodiments, such groups are substituted, e.g., wherein at least one instance of R^{C1} is halogen (i.e., —F, —Cl, —Br, or —I), —CN, —OR^{C1A} (e.g., —OH, —OCH₃), or unsubstituted C₁₋₃alkyl (e.g., —CH₃, —CH₂CH₃).

In certain embodiments, at least one of R^{B4}, R^{B5}, R^{B6}, and R^{B7} is substituted or unsubstituted 4-6 membered heterocyclyl. In certain embodiments, at least one of R^{B4}, R^{B5}, R^{B6}, and R^{B7} is a substituted or unsubstituted 4-membered heterocyclyl (e.g., azetidinyl), substituted or unsubstituted 5-membered heterocyclyl (e.g., pyrrolidinyl, pyrrolidin-2-one, oxazolidin-2-one), or substituted or unsubstituted 6-membered heterocyclyl (e.g., morpholinyl, piperidinyl, piperazinyl). In certain embodiments, such groups are unsubstituted by R^{C1}. In other embodiments, such groups are substituted, e.g., wherein at least one instance of R^{C1} is halogen (i.e., —F, —Cl, —Br, or —I), —CN, —OR^{C1A} (e.g., —OH, —OCH₃), or unsubstituted C₁₋₃alkyl (e.g., —CH₃, —CH₂CH₃).

In certain embodiments, at least one of R^{B4}, R^{B5}, R^{B6}, and R^{B7} is —C(═O)R^{B4A}C(═O)OR^{B4A}, —OC(═O)R^{B4A}, —OC(═O)OR^{B4A}, —NR^{B4B}C(═O)R^{B4A}, —NR^{B4B}C(═O)OR^{B4A}, or —S(O)₂R^{B4A}, wherein R^{B4A} is substituted or unsubstituted C₁₋₃alkyl, C₁₋₃haloalkyl, substituted or unsubstituted C₃₋₆ carbocyclyl, or substituted or unsubstituted 4- to 6-membered heterocyclyl, and R^{B4B} is as defined herein.

In certain embodiments, at least one of R^{B4}, R^{B5}, R^{B6}, and R^{B7} is —C(═O)R^{B4A}C(═O)OR^{B4A}, —OC(═O)R^{B4A}, —OC(═O)OR^{B4A}, —NR^{B4B}C(═O)R^{B4A}, —NR^{B4B}C(═O)OR^{B4A}, or —S(O)₂R^{B4A}, wherein R^{B4A} is unsubstituted C₁₋₃alkyl, i.e., unsubstituted C₁ alkyl (—CH₃), unsubstituted C₂ alkyl (—CH₂CH₃), or unsubstituted C₃ alkyl (—CH₂CH₂CH₃ or —CH(CH₃)₂).

In certain embodiments, at least one of R^{B4}, R^{B5}, R^{B6}, and R^{B7} is —C(═O)R^{B4A}, —C(═O)OR^{B4A}, —OC(═O)R^{B4A}, —OC(═O)OR^{B4A}, —NR^{B4B}C(═O)R^{B4A}, —NR^{B4B}C(═O)OR^{B4A}, or —S(O)₂R^{B4A}, wherein R^{B4A} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, at least one of R^{B4}, R^{B5}, R^{B6}, and R^{B7} is —C(═O)R^{B4A}, —C(═O)OR^{B4A}, —OC(═O)R^{B4A}, —OC(═O)OR^{B4A}, —NR^{B4B}C(═O)R^{B4A}, —NR^{B4B}C(═O)OR^{B4A}, or —S(O)₂R^{B4A}, wherein R^{B4A} is —CF₃, —CH₂F, —CHF₂, —CH₂Cl, —CH₂CF₃, —CH₂CHF₂, —CH(CH₃)CHF₂, or —CH(CH₃)CF₃.

In certain embodiments, at least one of R^{B4}, R^{B5}, R^{B6}, and R^{B7} is —C(═O)R^{B4A}C(═O)OR^{B4A}, —OC(═O)R^{B4A}, —OC(═O)OR^{B4A}, —NR^{B4B}C(═O)R^{B4A}, —NR^{B4B}C(═O)OR^{B4A}, or —S(O)₂R^{B4A}, wherein R^{B4A} is substituted or unsubstituted C₃₋₆carbocylyl or substituted or unsubstituted 4-6 membered heterocyclyl. In certain embodiments, at least one of R^{B4}, R^{B5}, R^{B6}, and R^{B7} is —C(═O)R^{B4A}, —C(═O)OR^{B4A}, —OC(═O)R^{B4A}, —OC(═O)OR^{B4A}, —NR^{B4B}C(═O)R^{B4A}, —NR^{B4B}C(═O)OR^{B4A}, or —S(O)₂R^{B4A}, wherein R^{B4A} is substituted or unsubstituted C₃carbocylyl (e.g., cyclopropyl). In certain embodiments, at least one of R^{B4}, R^{B5}, R^{B6}, and R^{B7} is —C(═O)R^{B4A}, —C(═O)OR^{B4A}, —OC(═O)R^{B4A}, —OC(═O)OR^{B4A}, —NR^{B4B}C(═O)R^{B4A}, —NR^{B4B}C(═O)OR^{B4A}, or —S(O)₂R^{B4A}, wherein R^{B4A} is substituted or unsubstituted 4-membered heterocyclyl (e.g., azetidinyl, oxetanyl). at least one of R^{B4}, R^{B5}, R^{B6}, and R^{B7} is —C(═O)R^{B4A}, —C(═O)OR^{B4A}, —OC(═O)R^{B4A}, —OC(═O)OR^{B4A}, —NR^{B4B}C(═O)R^{B4A}, —NR^{B4B}C(═O)OR^{B4A}, or —S(O)₂R^{B4A}, wherein R^{B4A} is substituted or unsubstituted 5-membered heterocyclyl (e.g., tetrahydrofuranyl). at least one of R^{B4}, R^{B5}, R^{B6}, and R^{B7} is —C(═O)R^{B4A}, —C(═O)OR^{B4A}, —OC(═O)R^{B4A}, —OC(═O)OR^{B4A}, —NR^{B4B}C(═O)R^{B4A}, —NR^{B4B}C(═O)OR^{B4A}, or —S(O)₂R^{B4A}, wherein R^{B4A} is substituted or unsubstituted 6-membered heterocyclyl (e.g., tetrahydropyranyl). In certain embodiments, such groups are unsubstituted by R^{C1}. In other embodiments, such groups are substituted, e.g., wherein at least one instance of R^{C1} is halogen (i.e., —F, —Cl, —Br, or —I), —CN, —OR^{C1A}(e.g., —OH, —OCH₃), or unsubstituted C₁₋₃alkyl (e.g., —CH₃, —CH₂CH₃).

In certain embodiments, at least one instance of R^{B4}, R^{B5}, R^{B6}, and R^{B7}, is C₁₋₃ alkyl, C₁₋₃haloalkyl, C₁₋₃ alkyl substituted with —OR^{C1B}, C₁₋₃ alkyl substituted with —N(R^{C1A})(R^{C1B}), C₁₋₃alkyl substituted with —CN, C₁₋₃ alkyl substituted with —C(═O)N(R^{C1A})(R^{C1B}), C₁₋₃ alkyl substituted with —C(═O)OR^{C1A}, —C(═O)N(R^{B4A})(R^{B4B}), —OC(═O)OR^{B1A}, —N(R^{B4A})(R^{B4B}), —OR^{B4B}, —SR^{B4B}, —S(O)₂R^{B4A}, —F, —Cl, —CN, substituted or unsubstituted C₃ carbocyclyl, or substituted or unsubstituted 4- to 6-membered heterocyclyl.

Various combination s of the above described embodiments of R^{B4}, R^{B5}, R^{B6}, and R^{B7} is further contemplated herein.

For example, in certain embodiments, wherein Ring B is of formula (xiv), various combinations of R^{B5} and R^{B7} are contemplated, e.g., wherein:
a. each instance of R^{B5} and R^{B7} is hydrogen; or
b. each instance of R^{B5} and R^{B7} is independently —OR^{B4B}; or
c. R^{B5} is —N(R^{B4A})(R^{B4B}), —OR^{B4B}, —SR^{B4B}, halogen, substituted or unsubstituted C₁₋₃ alkyl, C₁₋₃haloalkyl, substituted or unsubstituted C₃ carbocyclyl, or substituted or unsubstituted 4-6 membered heterocyclyl; and R^{B7} is hydrogen; or
d. R^{B5} is hydrogen and R^{B7} is —N(R^{B4A})(R^{B4B}), —OR^{B4B}, —SR^{B4B}, substituted or unsubstituted C₁₋₃ alkyl, C₁₋₃haloalkyl, or substituted or unsubstituted C₃ carbocyclyl.

In certain embodiments, wherein Ring B is of formula (xv), various combinations of R^{B5}, R^{B6}, and R^{B7} are contemplated, e.g., wherein:
a. each instance of R^{B5}, R^{B6}, and R^{B7} is hydrogen; or
b. R^{B5} is —N(R^{B4A})(R^{B4B}), —OR^{B4B}, —SR^{B4B}, halogen, substituted or unsubstituted C₁₋₃ alkyl, C₁₋₃haloalkyl, substituted or unsubstituted C₃ carbocyclyl, or substituted or unsubstituted 4-6 membered heterocyclyl, and R^{B6} and R^{B7} are hydrogen; or
c. R^{B6} is —N(R^{B4A})(R^{B4B}), —OR^{B4B}, —SR^{B4B}, halogen, substituted or unsubstituted C₁₋₃ alkyl, C₁₋₃haloalkyl, substituted or unsubstituted C₃ carbocyclyl, or substituted or unsubstituted 4-6 membered heterocyclyl, and R^{B5} and R^{B7} are hydrogen; or
d. R^{B7} is —N(R^{B4A})(R^{B4B}), —OR^{B4B}, —SR^{B4B}, halogen, substituted or unsubstituted C₁₋₃ alkyl, C₁₋₃haloalkyl, substituted or unsubstituted C₃ carbocyclyl, or substituted or unsubstituted 4-6 membered heterocyclyl, and R^{B5} and R^{B6} are hydrogen.

In certain embodiments, wherein Ring B is of formula (xvi), various combinations of R^{B4}, R^{B6} and R^{B7} are contemplated, e.g., wherein:
a. R^{B4} is —N(R^{B4A})(R^{B4B}), —OR^{B4B}, —SR^{B4B}, halogen, substituted or unsubstituted C₁₋₃ alkyl, C₁₋₃haloalkyl, substituted or unsubstituted C₃ carbocyclyl, or substituted or unsubstituted 4-6 membered heterocyclyl, and R^{B6} and R^{B7} are hydrogen; or
b. R^{B6} is —N(R^{B4A})(R^{B4B}), —OR^{B4B}, —SR^{B4B}, halogen, substituted or unsubstituted C₁₋₃ alkyl, C₁₋₃haloalkyl, substituted or unsubstituted C₃ carbocyclyl, or substituted or unsubstituted 4-6 membered heterocyclyl, and R^{B5} and R^{B7} are hydrogen; or
c. R^{B7} is —N(R^{B4A})(R^{B4B}), —OR^{B4B}, —SR^{B4B}, halogen, substituted or unsubstituted C₁₋₃ alkyl, C₁₋₃haloalkyl, substituted or unsubstituted C₃ carbocyclyl, or substituted or unsubstituted 4-6 membered heterocyclyl, and R^{B5} and R^{B6} are hydrogen; or
d. R^{B4} and R^{B6} are —N(R^{B4A})(R^{B4B}), —OR^{B4B}, —SR^{B4B}, halogen, substituted or unsubstituted C₁₋₃ alkyl, C₁₋₃haloalkyl, substituted or unsubstituted C₃ carbocyclyl, or substituted or unsubstituted 4-6 membered heterocyclyl, and R^{B7} is hydrogen.

In certain embodiments, wherein Ring B is of formula (xvii), various combinations of R^{B5} and R^{B6} are contemplated, e.g., wherein:
a. R^{B5} is —N(R^{B4A})(R^{B4B}), —OR^{B4B}, —SR^{B4B}, halogen, substituted or unsubstituted C₁₋₃ alkyl, C₁₋₃haloalkyl, substituted or unsubstituted C₃ carbocyclyl, or substituted or unsubstituted 4-6 membered heterocyclyl, and R^{B6} is hydrogen; or
b. R^{B6} is —N(R^{B4A})(R^{B4B}), —OR^{B4B}, —SR^{B4B}, halogen, substituted or unsubstituted C₁₋₃ alkyl, C₁₋₃haloalkyl, substituted or unsubstituted C₃ carbocyclyl, or substituted or unsubstituted 4-6 membered heterocyclyl, and R^{B5} is hydrogen.

Furthermore, as generally defined herein, in certain embodiments, each instance of R^{B3} is independently hydrogen, unsubstituted C₁₋₃alkyl, or C₁₋₃haloalkyl, provided at least one instance of R^{B3} is hydrogen. In certain embodiments, each instance of R^{B3} is hydrogen. In certain embodiments, one instance of R^{B3} is unsubstituted C₁₋₃alkyl (e.g., —CH₃) or C₁₋₃haloalkyl (e.g., —CF₃). R^{B3} is hydrogen or —CH₃, provided at least one instance of R^{B3} is hydrogen.

Furthermore, as generally defined herein, in certain embodiments, each instance of R^{N2} and R^{B8} is independently substituted or unsubstituted C₁₋₃alkyl or C₁₋₃haloalkyl, or R^{N2} and R^{B8} are joined to form a substituted or unsubstituted 5- to 6-membered ring.

In certain embodiments, each instance of R^{N2} and R^{B8} is independently substituted or unsubstituted C₁₋₃alkyl or C₁₋₃haloalkyl.

In certain embodiments, at least one of R^{N2} and R^{B8} is substituted or unsubstituted C₁₋₃alkyl, i.e., a C₁₋₃alkyl substituted by 1, 2, or 3 R^{C1} groups as previously described herein, or an unsubstituted C₁₋₃alkyl. In certain embodiments, at least one of R^{N2} and R^{B8} is unsubstituted C₁₋₃alkyl, i.e., C₁ alkyl (—CH₃), unsubstituted C₂ alkyl (—CH₂CH₃), or unsubstituted C₃ alkyl (—CH₂CH₂CH₃ or —CH(CH₃)₂). In certain embodiments, at least one of R^{N2} and R^{B8} is unsubstituted C₁₋₃alkyl of formula —CH₃, —CH₂CH₃, or —CH(CH₃)₂. In certain embodiments, at least one of R^{N2} and R^{B8} is substituted C₁₋₃alkyl, e.g., of formula:

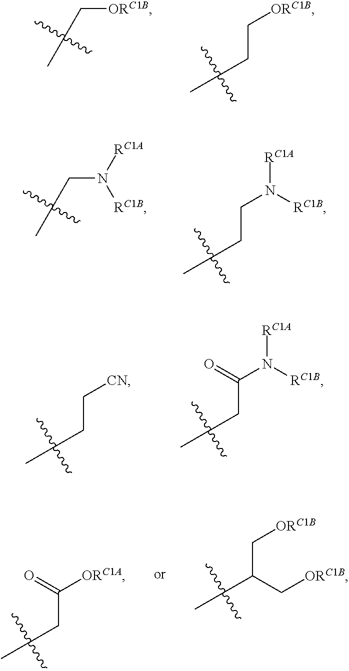

wherein R^{C1A} and R^{C1B} are as defined herein.

In certain embodiments, at least one of R^{N2} and R^{B8} is C₁₋₃haloalkyl, e.g., C₁ haloalkyl (—CF₃, —CCl₃, —CFCl₂, —CF₂Cl, —CH₂F, —CHF₂, —CH₂Cl, CHCl₂), C₂ haloalkyl (—CF₂CF₃, —CH₂CF₃, —CH₂CHF₂, —CH₂CH₂F, —CH₂CCl₃, —CH₂CHCl₂, —CH₂CH₂Cl), or C₃ haloalkyl (—CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃). In certain embodiments, at least one of R^{N2} and R^{B8} is —CF₃, —CH₂F, —CHF₂, —CH₂Cl, —CH₂CF₃, —CH₂CHF₂, —CH(CH₃)CHF₂, or —CH(CH₃)CF₃.

Alternatively, in certain embodiments, R^{N2} and R^{B8} are joined to form a substituted or unsubstituted 5- to 6-membered ring. In certain embodiments, R^{N2} and R^{B8} are joined to form a substituted or unsubstituted 5-membered ring. In certain embodiments, R^{N2} and R^{B8} are joined to form a substituted or unsubstituted 6-membered ring. In certain embodiments, R^{N2} and R^{B8} are joined to form an unsubstituted ring.

In certain embodiments, each instance of R^{B8} and R^{N2} is independently —CH₃, —CH₂CH₃, —CH₂CH₂CH₃, —CH(CH₃)₂, —CF₃, —CCl₃, —CFCl₂, —CF₂Cl, —CH₂F, —CHF₂, —CH₂Cl, —CHCl₂, —CF₂CF₃, —CH₂CF₃, —CH₂CHF₂, —CH₂CH₂F, —CH₂CCl₃, —CH₂CHCl₂, —CH₂CH₂Cl, —CF₂CF₂CF₃, —CH₂CF₂CF₃, —CH₂CH₂CF₃, —CH₂CH₂CHF₂, —CH₂CH₂CH₂F, —CH₂CH₂CCl₃, —CH₂CH₂CHCl₂, —CH₂CH₂CH₂Cl, —CH(CH₃)CHF₂, or —CH(CH₃)CF₃; or R^{N2} and R^{B8} are joined to form an unsubstituted 5-membered ring.

In certain embodiments, Ring B is of formula:

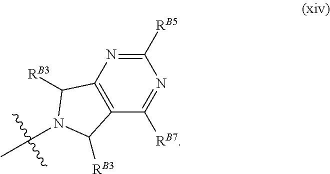

In certain embodiments of formula (xiv), Ring B is of formula:

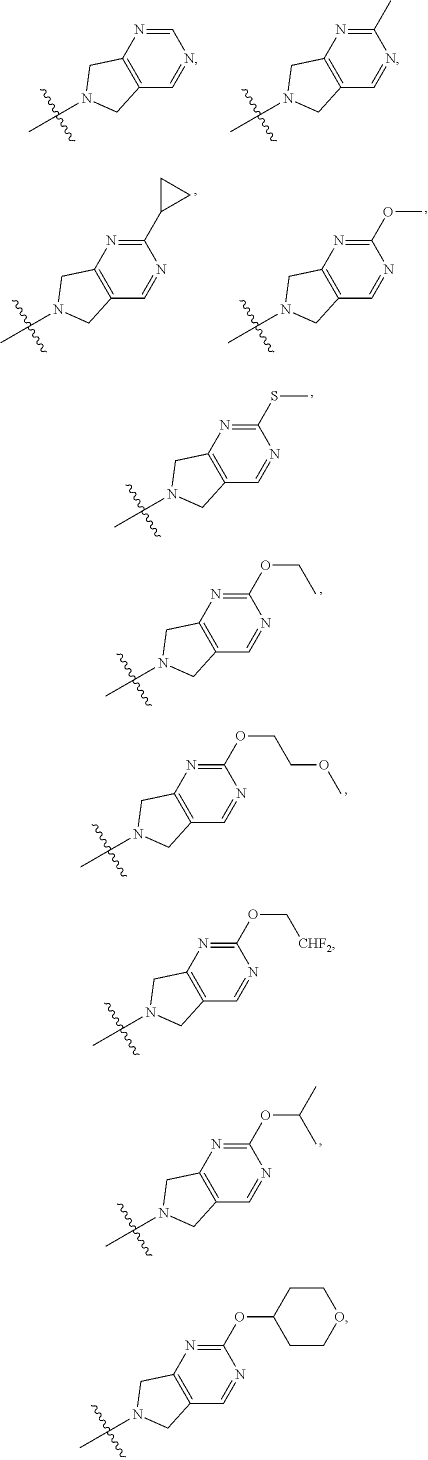

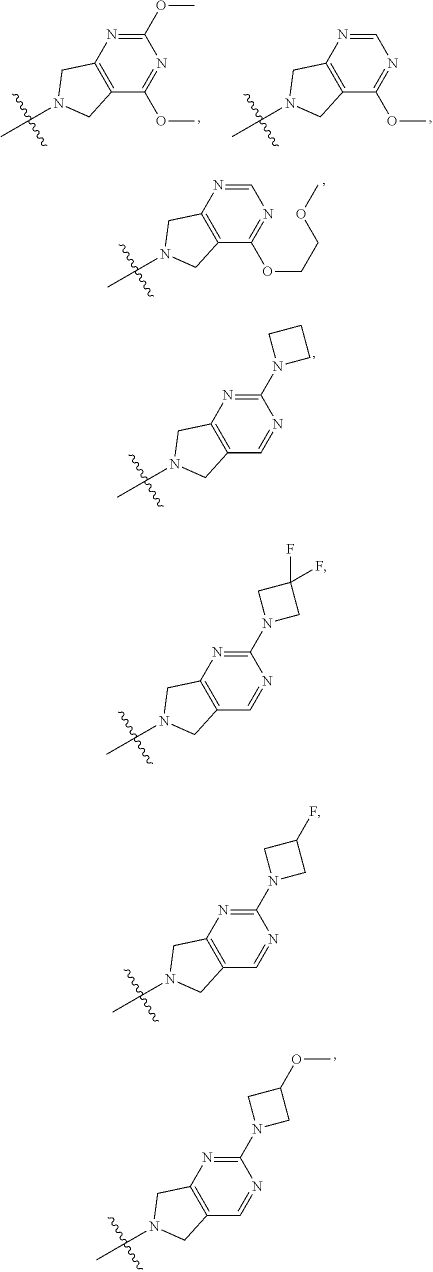

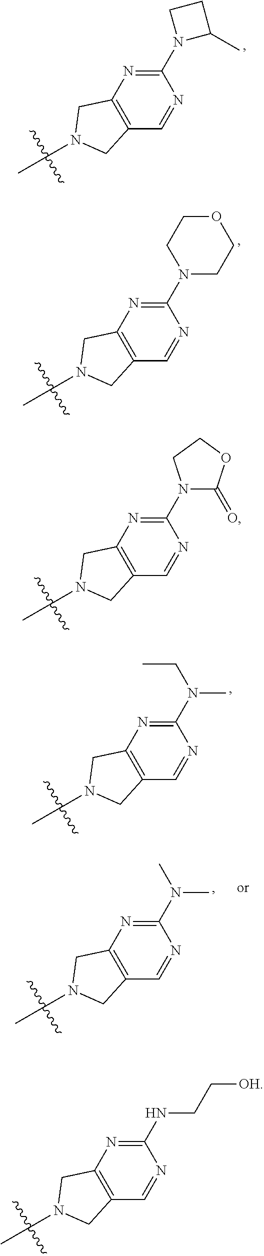 [SMILES: *N1Cc2cnc(N3CCC3C)nc2C1 ; *N1Cc2cnc(N3CCOCC3)nc2C1 ; *N1Cc2cnc(N3CCOC3=O)nc2C1 ; *N1Cc2cnc(N(C)CC)nc2C1 ; *N1Cc2cnc(N(C)C)nc2C1 ; *N1Cc2cnc(NCCO)nc2C1]

In certain embodiments, Ring B is of formula:

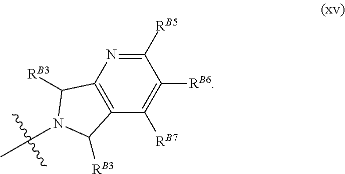

In certain embodiments of formula (xv), Ring B is of formula:

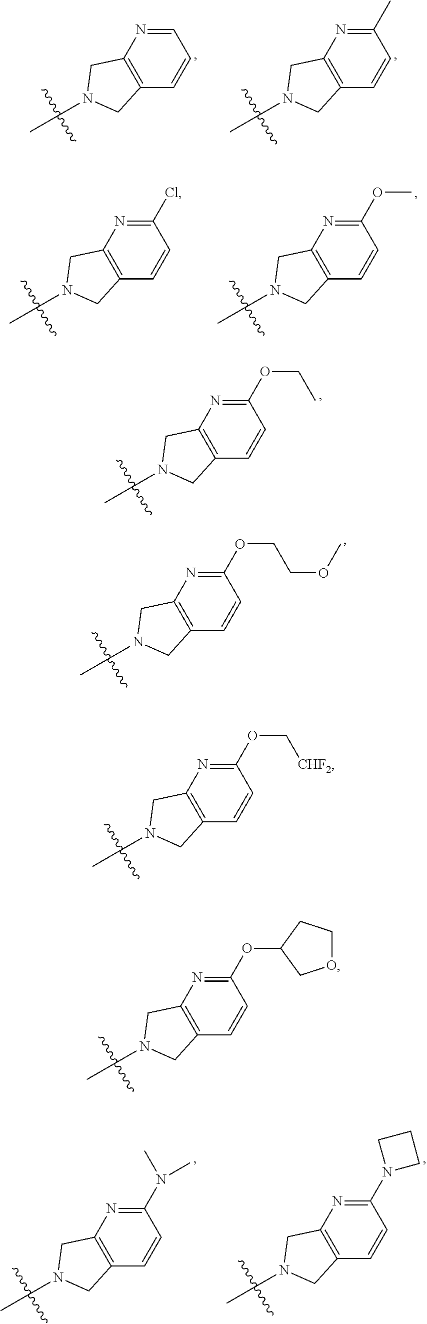

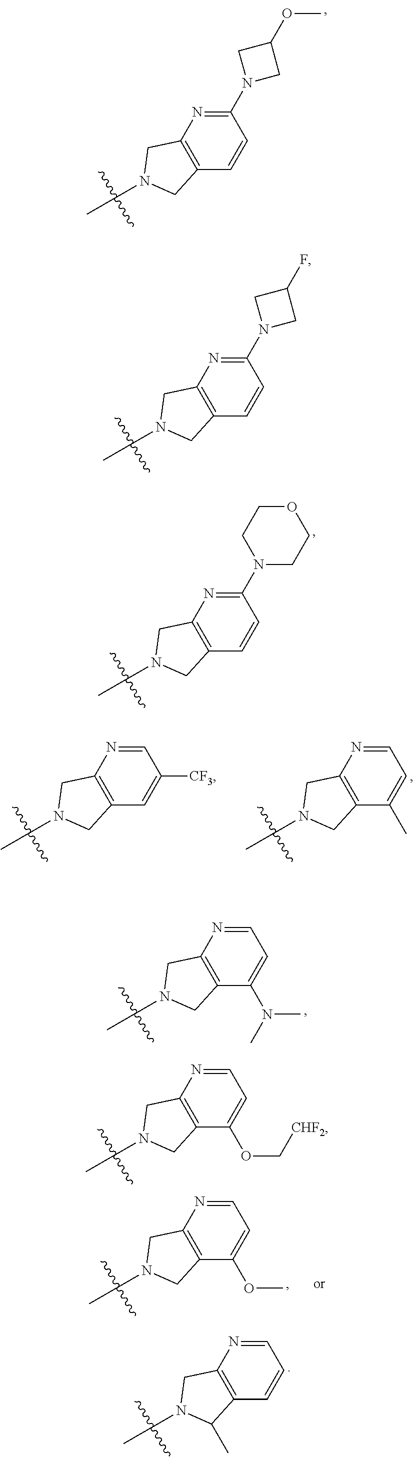

In certain embodiments, Ring B is of formula:

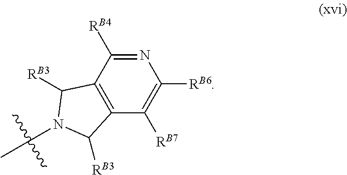

In certain embodiments of formula (xvi), Ring B is of formula:

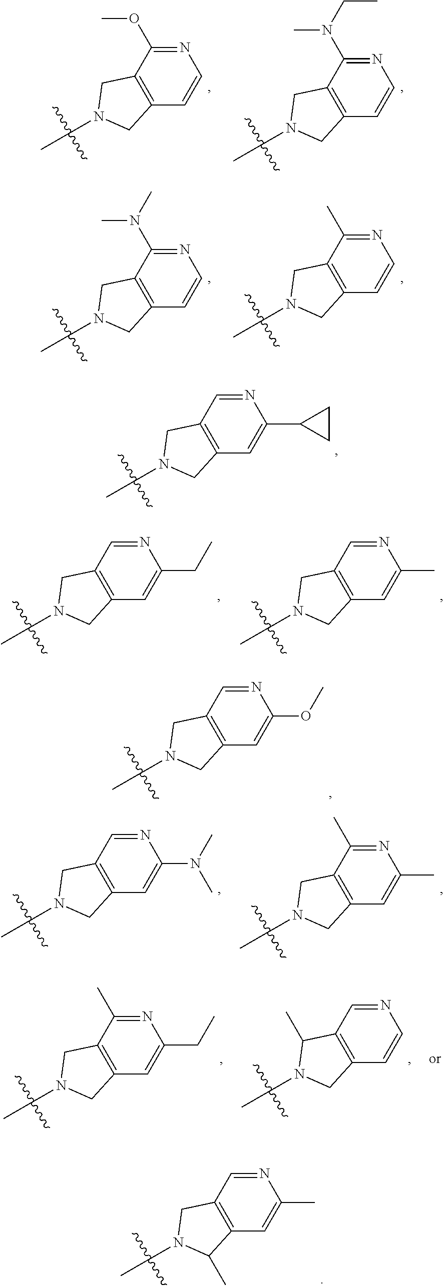

In certain embodiments, Ring B is of formula:

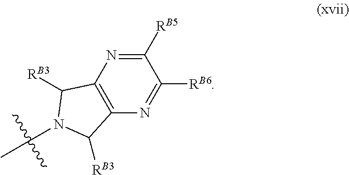

In certain embodiments of formula (xvii), Ring B is of formula:

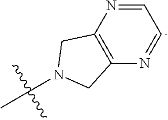 [SMILES: *N1Cc2nccnc2C1]

In certain embodiments, Ring B is of formula:

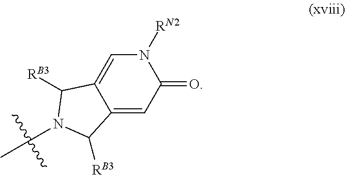

In certain embodiments of formula (xviii), Ring B is of formula:

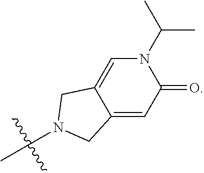 [SMILES: *N1Cc2cc(=O)n(C(C)C)cc2C1]

In certain embodiments, Ring B is of formula:

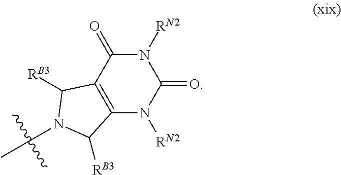

In certain embodiments of formula (xix), Ring B is of formula:

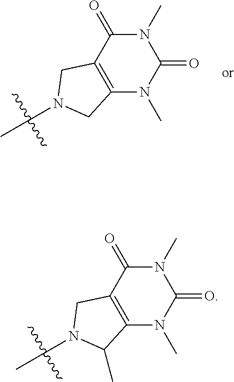 [SMILES: *N1Cc2c(n(C)c(=O)n(C)c2=O)C1 ; "or" ; *N1Cc2c(n(C)c(=O)n(C)c2=O)C1C]

In certain embodiments, Ring B is of formula:

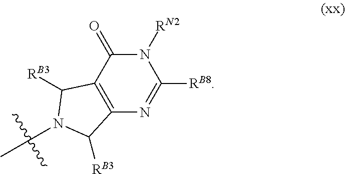

In certain embodiments of formula (xx), Ring B is of formula:

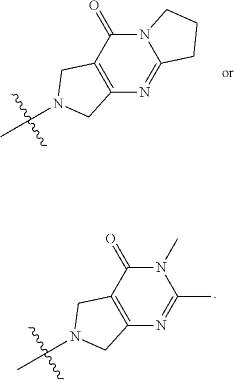 [SMILES: *N1Cc2nc3n(c(=O)c2C1)CCC3 ; "or" ; *N1Cc2nc(C)n(C)c(=O)c2C1]

In certain embodiments, Ring B is of formula:

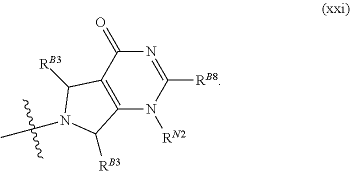

In certain embodiments of formula (xxi), Ring B is of formula:

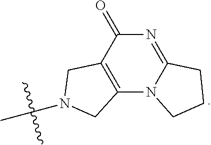 [SMILES: *N1Cc2c(n3c(nc2=O)CCC3)C1]

### (VIII) Other Ring B Groups

Other Ring B groups contemplated herein include Ring B groups of formula (xxv) and (xxix). For example, in certain embodiments, Ring B is of formula:

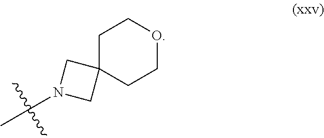 [SMILES: *N1CC2(CCOCC2)C1]

In certain embodiments, Ring B is of formula:

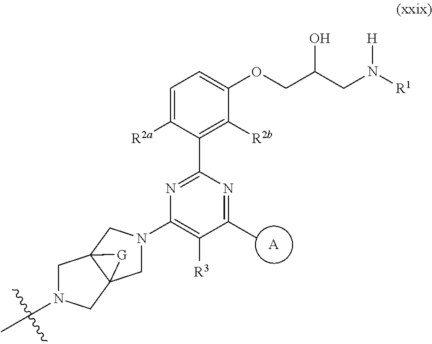

wherein R¹, R^{2a}, R^{2b}, R³, Ring A, and [symbol] are as defined herein.

In certain embodiments,

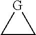

represents a double or single bond (e.g., represented by [symbol] ) to provide a Ring B of formula:

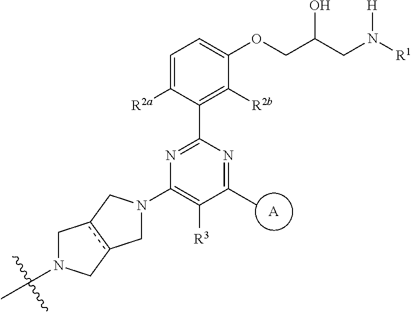

In certain embodiments of formula (xxix), [symbol] represents a single bond. In certain embodiments of formula (xxix), [symbol] represents a single bond, and the ring fusion is in the trans configuration. In certain embodiments of formula (xxix), [symbol] represents a single bond, and the ring fusion is in the cis configuration. In certain embodiments of formula (xxix), [symbol] represents a double bond.

In certain embodiments of formula (xxix), G of

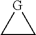

is —CH₂— to provide a cyclopropanated Ring B of formula:

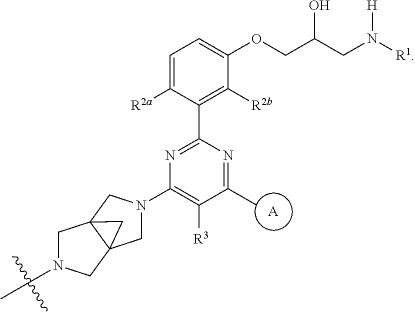

In certain embodiments of formula (xxix), Ring B is of formula:

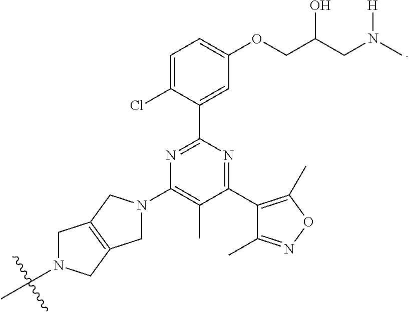 [SMILES: *N1CC2=C(C1)CN(c1nc(-c3cc(OCC(O)CNC)ccc3Cl)nc(-c3c(C)noc3C)c1C)C2]

### (IX) Various Contemplated Combinations of Specific Embodiments

Various combinations of specific embodiments as described herein are specifically contemplated.

For example, in certain embodiments, wherein Ring B is of formula (i), and wherein Ring A is of formula (A-i), and wherein each of R^{A1} and R^{A2} is —CH₃, provided is a compound of formula:

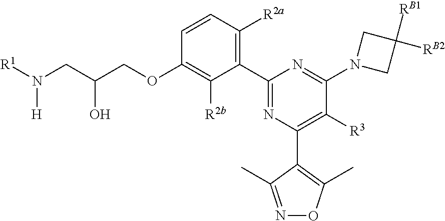

or a pharmaceutically acceptable salt thereof.

In certain embodiments, Ring B is of formula (i), wherein Ring A is of formula (A-ii), and wherein R^{A5} is —CH₃, R^{A4} is —Br, and R^{A3} is —CH₃, provided is a compound of formula:

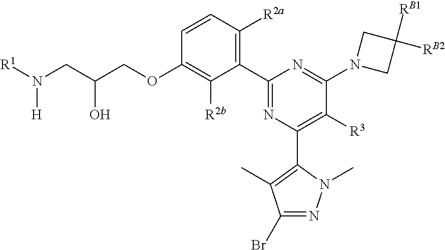

or a pharmaceutically acceptable salt thereof.

In certain embodiments, Ring B is of formula (ii), wherein Ring A is of formula (A-i), and wherein each of R^{A1} and R^{A2} is —CH₃, provided is a compound of formula:

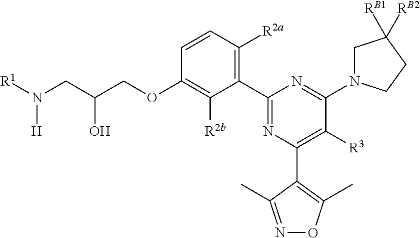

or a pharmaceutically acceptable salt thereof.

In certain embodiments, Ring B is of formula (iii), wherein Ring A is of formula (A-i), and wherein each of R^{A1} and R^{A2} is —CH₃, provided is a compound of formula:

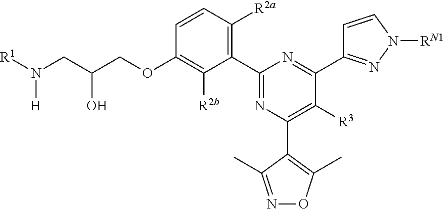

or a pharmaceutically acceptable salt thereof.

In certain embodiments, Ring B is of formula (iv), wherein Ring A is of formula (A-i), and wherein each of R^{A1} and R^{A2} is —CH₃, provided is a compound of formula:

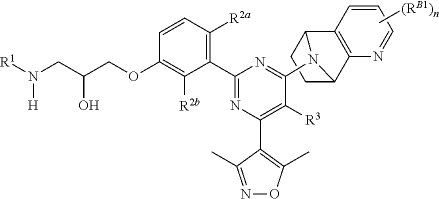

or a pharmaceutically acceptable salt thereof.

In certain embodiments, Ring B is of formula (v), wherein Ring A is of formula (A-i), and wherein each of R^{A1} and R^{A2} is —CH₃, provided is a compound of formula:

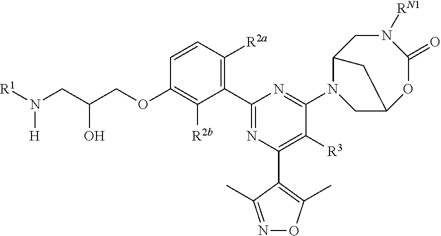

or a pharmaceutically acceptable salt thereof.

In certain embodiments, Ring B is of formula (vi), wherein Ring A is of formula (A-i), and wherein each of R^{A1} and R^{A2} is —CH₃, provided is a compound of formula:

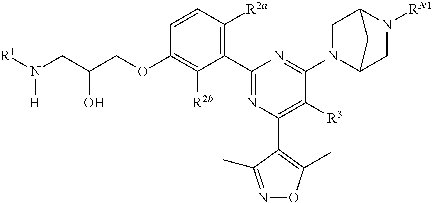

or a pharmaceutically acceptable salt thereof.

In certain embodiments, Ring B is of formula (vii), wherein Ring A is of formula (A-i), and wherein each of R^{A1} and R^{A2} is —CH₃, provided is a compound of formula:

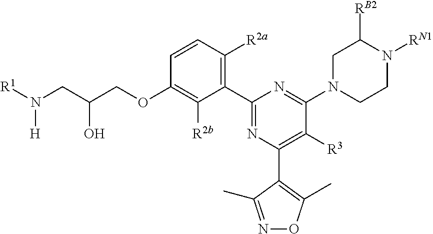

or a pharmaceutically acceptable salt thereof.

In certain embodiments, Ring B is of formula (viii), wherein Ring A is of formula (A-i), and wherein each of R^{A1} and R^{A2} is —CH₃, provided is a compound of formula:

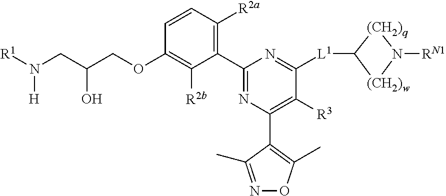

or a pharmaceutically acceptable salt thereof.

In certain embodiments, Ring B is of formula (viii), wherein Ring A is of formula (A-iii), and wherein each of R^{A3} and R^{A5} is —CH₃, provided is a compound of formula:

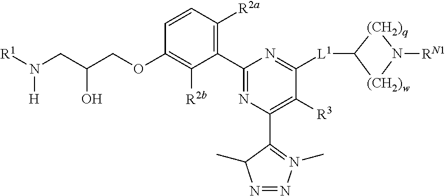

or a pharmaceutically acceptable salt thereof.

In certain embodiments, Ring B is of formula (ix), wherein Ring A is of formula (A-i), and wherein each of R^{A1} and R^{A2} is —CH₃, provided is a compound of formula:

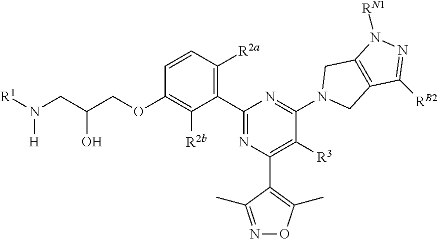

or a pharmaceutically acceptable salt thereof.

In certain embodiments, Ring B is of formula (ix), wherein Ring A is of formula (A-ii), and wherein R^{A3} is —Cl, R^{A4} is hydrogen, and R^{A5} is —CH₃, provided is a compound of formula:

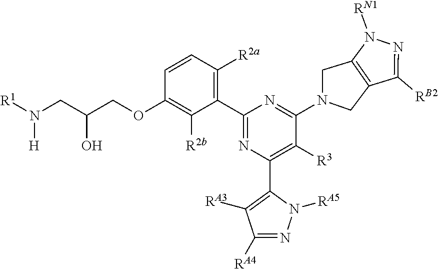

or a pharmaceutically acceptable salt thereof.

In certain embodiments, Ring B is of formula (x), wherein Ring A is of formula (A-i), and wherein each of R^{A1} and R^{A2} is —CH₃, provided is a compound of formula:

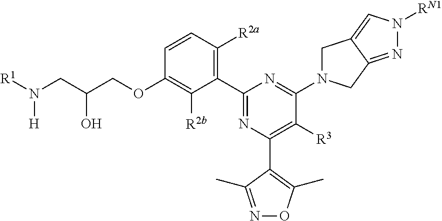

or a pharmaceutically acceptable salt thereof.

In certain embodiments, Ring B is of formula (x), wherein Ring A is of formula (A-ii), and wherein R^{A3} is —Cl, R^{A4} is hydrogen, and R^{A5} is —CH₃, provided is a compound of formula:

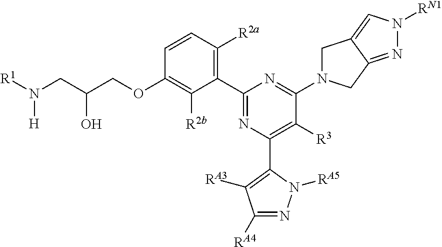

or a pharmaceutically acceptable salt thereof.

In certain embodiments, Ring B is of formula (xi), wherein Ring A is of formula (A-i), and wherein each of R^{A1} and R^{A2} is —CH₃, provided is a compound of formula:

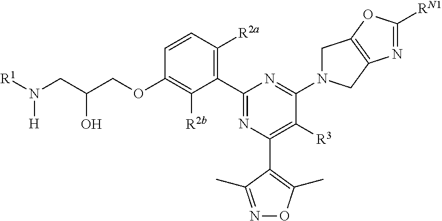

or a pharmaceutically acceptable salt thereof.

In certain embodiments, Ring B is of formula (xii), wherein Ring A is of formula (A-i), and wherein each of R^{A1} and R^{A2} is —CH₃, provided is a compound of formula:

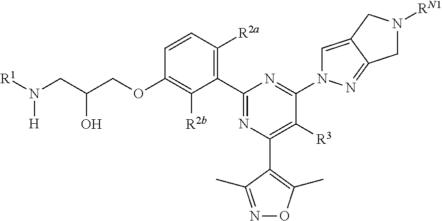

or a pharmaceutically acceptable salt thereof.

In certain embodiments, Ring B is of formula (xiii), wherein Ring A is of formula (A-i), and wherein each of R^{A1} and R^{A2} is —CH₃, provided is a compound of formula:

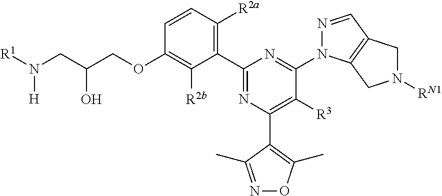

or a pharmaceutically acceptable salt thereof.

In certain embodiments, Ring B is of formula (xiv), wherein Ring A is of formula (A-i), and wherein each of R^{A1} and R^{A2} is —CH₃, provided is a compound of formula:

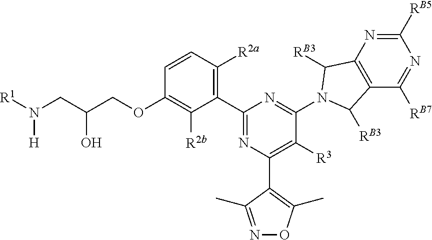

or a pharmaceutically acceptable salt thereof.

In certain embodiments, Ring B is of formula (xiv), wherein Ring A is of formula (A-i), and wherein R^{A1} is —CH₃ and R^{A2} is —CH₂CH₃, provided is a compound of formula:

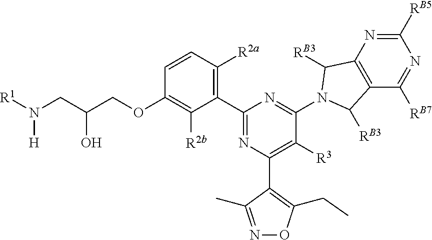

or a pharmaceutically acceptable salt thereof.

In certain embodiments, Ring B is of formula (xiv), wherein Ring A is of formula (A-ii), and wherein R^{A3} is —CH₃, R^{A4} is —Cl, and R^{A5} is —CH₃, provided is a compound of formula:

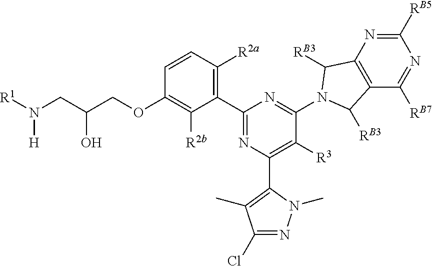

or a pharmaceutically acceptable salt thereof.

In certain embodiments, Ring B is of formula (xiv), wherein Ring A is of formula (A-ii), and wherein R^{A3} is —CH₃, R^{A4} is —Br, and R^{A5} is —CH₃, provided is a compound of formula:

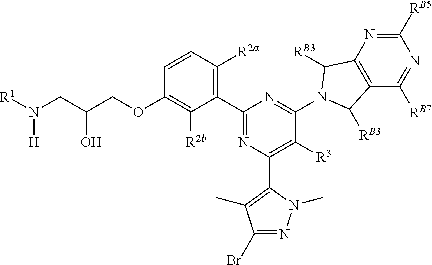

or a pharmaceutically acceptable salt thereof.

In certain embodiments, Ring B is of formula (xiv), wherein Ring A is of formula (A-ii), and wherein R^{A3} is —CH₃, R^{A4} is —CN, and R^{A5} is —CH₃, provided is a compound of formula:

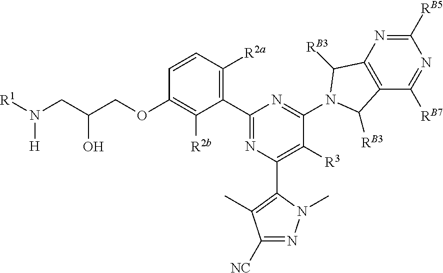

or a pharmaceutically acceptable salt thereof.

In certain embodiments, Ring B is of formula (xiv), wherein Ring A is of formula (A-ii), and wherein R^{A3} is —CH₃, R^{A4} is hydrogen, and R^{A5} is —CH₃, provided is a compound of formula:

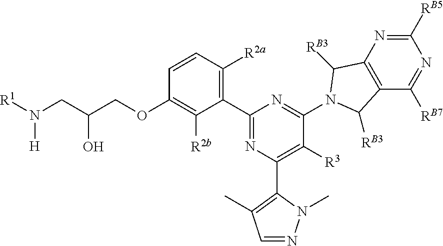

or a pharmaceutically acceptable salt thereof.

In certain embodiments, Ring B is of formula (xiv), wherein Ring A is of formula (A-ii), and wherein R^{A3} is —CH₃, R^{A4} is hydrogen, and R^{A5} is —CH₂CH₃, provided is a compound of formula:

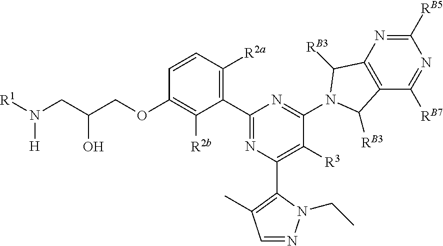

or a pharmaceutically acceptable salt thereof.

In certain embodiments, Ring B is of formula (xiv), wherein Ring A is of formula (A-ii), and wherein R^{A3} is —Cl, R^{A4} is hydrogen, and R^{A5} is —CH₃, provided is a compound of formula:

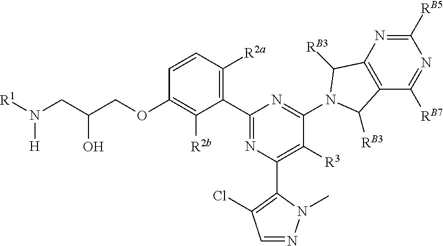

or a pharmaceutically acceptable salt thereof.

In certain embodiments, Ring B is of formula (xiv), wherein Ring A is of formula (A-ii), and wherein R^{A3} is —Br, R^{A4} is hydrogen, and R^{A5} is —CH₃, provided is a compound of formula:

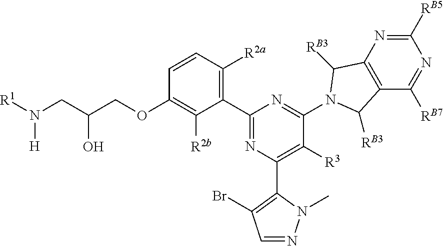

or a pharmaceutically acceptable salt thereof.

In certain embodiments, Ring B is of formula (xiv), wherein Ring A is of formula (A-ii), and wherein R^{A3} is —CN, R^{A4} is hydrogen, and R^{A5} is —CH₃, provided is a compound of formula:

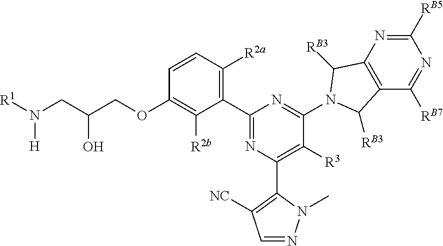

or a pharmaceutically acceptable salt thereof.

In certain embodiments, Ring B is of formula (xv), wherein Ring A is of formula (A-i), and wherein each of R^{A1} and R^{A2} is —CH₃, provided is a compound of formula:

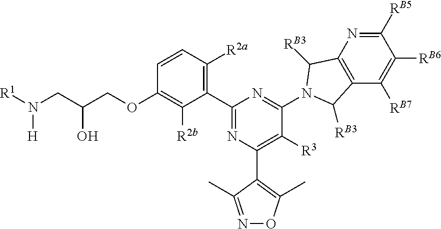

or a pharmaceutically acceptable salt thereof.

In certain embodiments, Ring B is of formula (xv), wherein Ring A is of formula (A-i), and wherein R^{A1} is —CH₃ and R^{A2} is —CH₂CH₃, provided is a compound of formula:

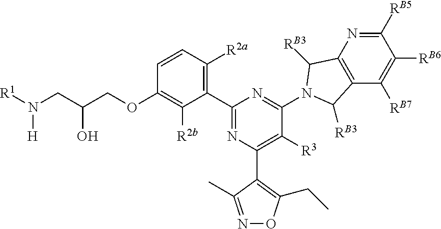

or a pharmaceutically acceptable salt thereof.

In certain embodiments, Ring B is of formula (xv), wherein Ring A is of formula (A-i), and wherein R^{A1} is —CH₂CH₃ and R^{A2} is —CH₃, provided is a compound of formula:

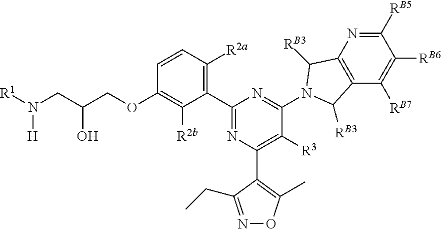

or a pharmaceutically acceptable salt thereof.

In certain embodiments, Ring B is of formula (xv), wherein Ring A is of formula (A-ii), and wherein R^{A3} is —CH₃, R^{A4} is —Br, and R^{A5} is —CH₃, provided is a compound of formula:

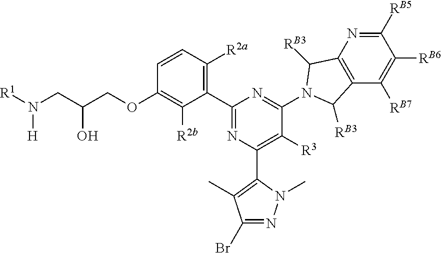

or a pharmaceutically acceptable salt thereof.

In certain embodiments, Ring B is of formula (xv), wherein Ring A is of formula (A-ii), and wherein R^{A3} is —CH₃, R^{A4} is —Cl, and R^{A5} is —CH₃, provided is a compound of formula:

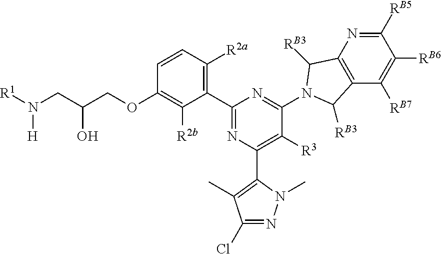

or a pharmaceutically acceptable salt thereof.

In certain embodiments, Ring B is of formula (xv), wherein Ring A is of formula (A-ii), and wherein R^{A3} is —Br, R^{A4} is -hydrogen, and R^{A5} is —CH₃, provided is a compound of formula:

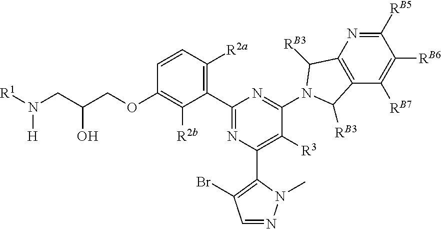

or a pharmaceutically acceptable salt thereof.

In certain embodiments, Ring B is of formula (xv), wherein Ring A is of formula (A-ii), and wherein R^{A3} is —I, R^{A4} is -hydrogen, and R^{A5} is —CH₃, provided is a compound of formula:

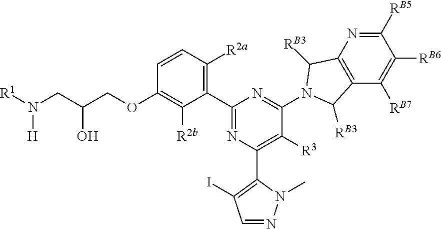

or a pharmaceutically acceptable salt thereof.

In certain embodiments, Ring B is of formula (xv), wherein Ring A is of formula (A-ii), and wherein R^{A3} is —Cl, R^{A4} is -hydrogen, and R^{A5} is —CH₃, provided is a compound of formula:

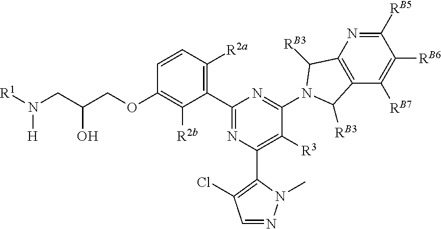

or a pharmaceutically acceptable salt thereof.

In certain embodiments, Ring B is of formula (xv), wherein Ring A is of formula (A-ii), and wherein R^{A3} is —CN, R^{A4} is -hydrogen, and R^{A5} is —CH₃, provided is a compound of formula:

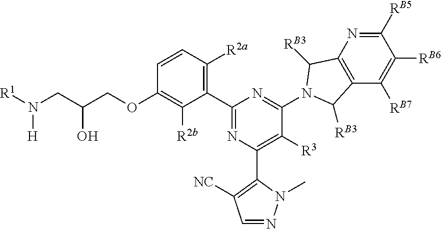

or a pharmaceutically acceptable salt thereof.

In certain embodiments, Ring B is of formula (xv), wherein Ring A is of formula (A-ii), and wherein R^{A3} is —CH₃, R^{A4} is -hydrogen, and R^{A5} is —CH₃, provided is a compound of formula:

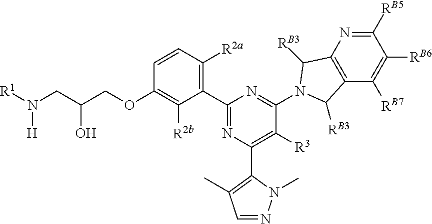

or a pharmaceutically acceptable salt thereof.

In certain embodiments, Ring B is of formula (xv), wherein Ring A is of formula (A-ii), and wherein R^{A3} is —Cl, R^{A4} is -hydrogen, and R^{A5} is —CH₂CH₃, provided is a compound of formula:

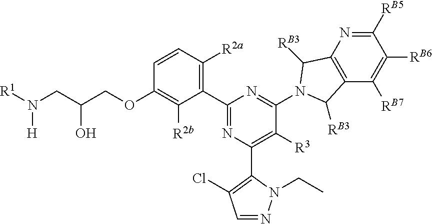

or a pharmaceutically acceptable salt thereof.

In certain embodiments, Ring B is of formula (xv), wherein Ring A is of formula (A-ii), and wherein R^{A3} is —CH₃, R^{A4} is -hydrogen, and R^{A5} is —CH₂CH₃, provided is a compound of formula:

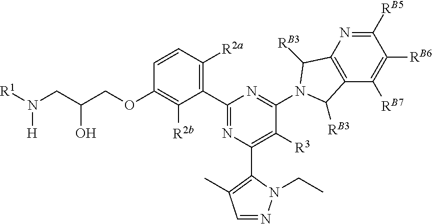

or a pharmaceutically acceptable salt thereof.

In certain embodiments, Ring B is of formula (xvi), wherein Ring A is of formula (A-i), and wherein each of R^{A1} and R^{A2} is —CH₃, provided is a compound of formula:

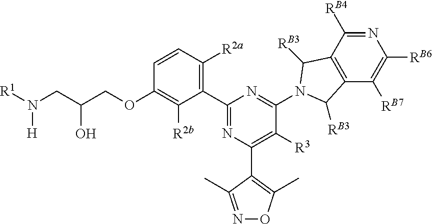

or a pharmaceutically acceptable salt thereof.

In certain embodiments, Ring B is of formula (xvii), wherein Ring A is of formula (A-ii), and wherein R^{A3} is —Cl, R^{A4} is -hydrogen, and R^{A5} is —CH₃, provided is a compound of formula:

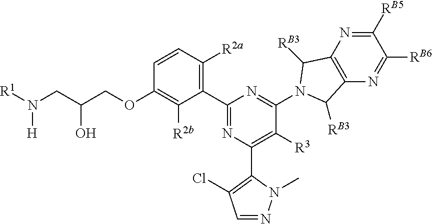

or a pharmaceutically acceptable salt thereof.

In certain embodiments, Ring B is of formula (xviii), wherein Ring A is of formula (A-i), and wherein each of R^{A1} and R^{A2} is —CH₃, provided is a compound of formula:

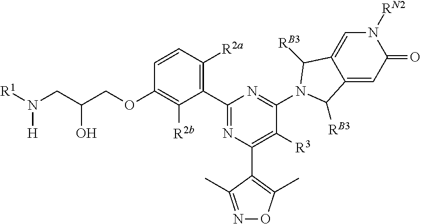

or a pharmaceutically acceptable salt thereof.

In certain embodiments, Ring B is of formula (xix), wherein Ring A is of formula (A-i), and wherein each of R^{A1} and R^{A2} is —CH₃, provided is a compound of formula:

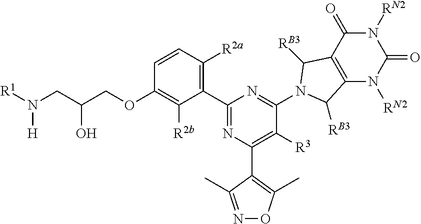

or a pharmaceutically acceptable salt thereof.

In certain embodiments, Ring B is of formula (xx), wherein Ring A is of formula (A-i), and wherein each of R^{A1} and R^{A2} is —CH₃, provided is a compound of formula:

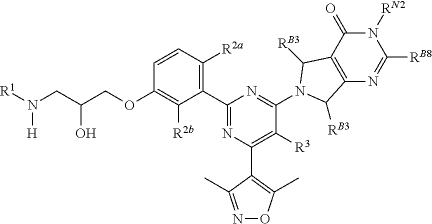

or a pharmaceutically acceptable salt thereof.

In certain embodiments, Ring B is of formula (xxi), wherein Ring A is of formula (A-i), and wherein each of R^{A1} and R^{A2} is —CH₃, provided is a compound of formula:

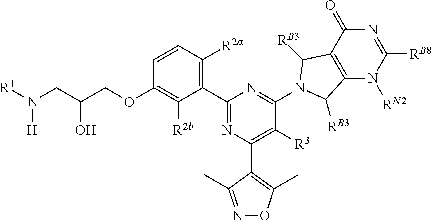

or a pharmaceutically acceptable salt thereof.

In certain embodiments, Ring B is of formula (xxii), wherein Ring A is of formula (A-i), and wherein each of R^{A1} and R^{A2} is —CH₃, provided is a compound of formula:

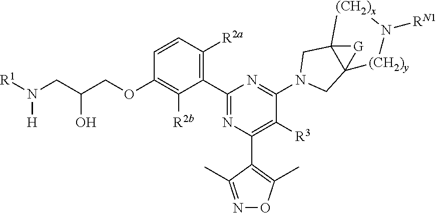

or a pharmaceutically acceptable salt thereof.

In certain embodiments, Ring B is of formula (xxii), wherein Ring A is of formula (A-ii), and wherein R^{A3} is —Cl, R^{A4} is -hydrogen, and R^{A5} is —CH₃, provided is a compound of formula:

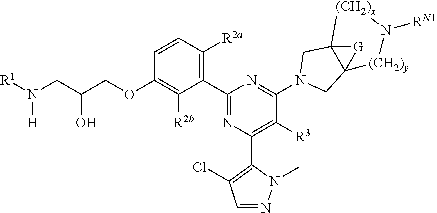

or a pharmaceutically acceptable salt thereof.

In certain embodiments, Ring B is of formula (xxii), wherein Ring A is of formula (A-ii), and wherein R^{A3} is —Br, R^{A4} is -hydrogen, and R^{A5} is —CH₃, provided is a compound of formula:

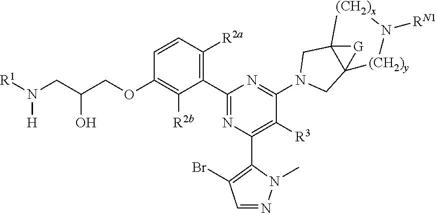

or a pharmaceutically acceptable salt thereof.

In certain embodiments, Ring B is of formula (xxiii), wherein Ring A is of formula (A-i), and wherein each of R^{A1} and R^{A2} is —CH₃, provided is a compound of formula:

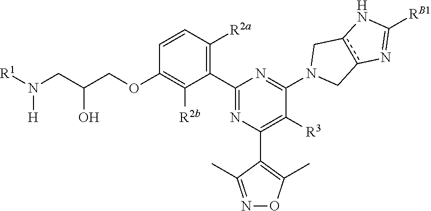

or a pharmaceutically acceptable salt thereof.

In certain embodiments, Ring B is of formula (xxiv), wherein Ring A is of formula (A-i), and wherein each of R^{A1} and R^{A2} is —CH₃, provided is a compound of formula:

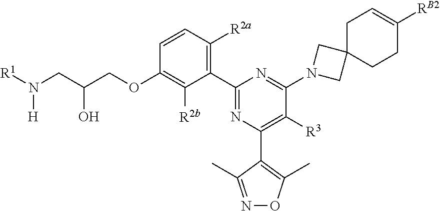

or a pharmaceutically acceptable salt thereof.

In certain embodiments, Ring B is of formula (xxv), wherein Ring A is of formula (A-i), and wherein each of R^{A1} and R^{A2} is —CH₃, provided is a compound of formula:

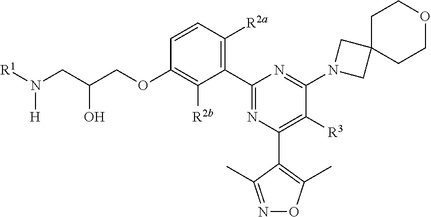

or a pharmaceutically acceptable salt thereof.

In certain embodiments, Ring B is of formula (xxvi), wherein Ring A is of formula (A-i), and wherein each of R^{A1} and R^{A2} is —CH₃, provided is a compound of formula:

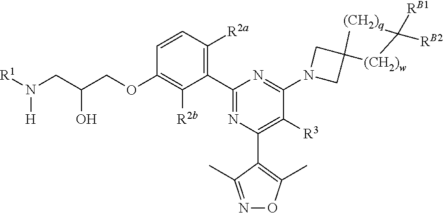

or a pharmaceutically acceptable salt thereof.

In certain embodiments, Ring B is of formula (xxvi), wherein Ring A is of formula (A-i), and wherein R^{A1} is —CH₃ and R^{A2} is —CH₃, provided is a compound of formula:

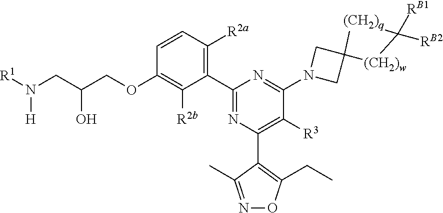

or a pharmaceutically acceptable salt thereof.

In certain embodiments, Ring B is of formula (xxvi), wherein Ring A is of formula (A-ii), and wherein R^{A3} is —CH₃, R^{A4} is —Cl, and R^{A5} is —CH₃, provided is a compound of formula:

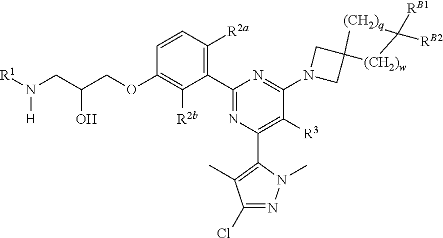

or a pharmaceutically acceptable salt thereof.

In certain embodiments, Ring B is of formula (xxvi), wherein Ring A is of formula (A-ii), and wherein R^{A3} is —CH₃, R^{A4} is —Br, and R^{A5} is —CH₃, provided is a compound of formula:

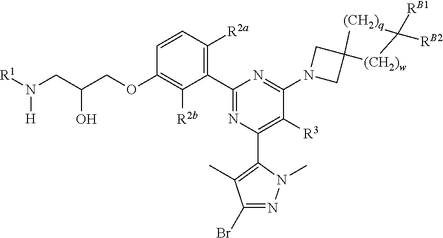

or a pharmaceutically acceptable salt thereof.

In certain embodiments, Ring B is of formula (xxvii), wherein Ring A is of formula (A-i), and wherein each of R^{A1} and R^{A2} is —CH₃, provided is a compound of formula:

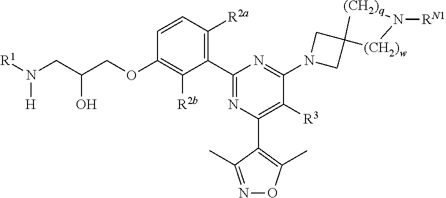

or a pharmaceutically acceptable salt thereof.

In certain embodiments, Ring B is of formula (xxvii), wherein Ring A is of formula (A-i), and wherein R^{A1} is —CH₃ and R^{A2} is —CH₂CH₃, provided is a compound of formula:

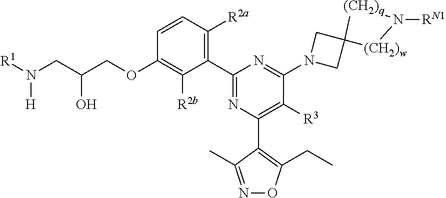

or a pharmaceutically acceptable salt thereof.

In certain embodiments, Ring B is of formula (xxvii), wherein Ring A is of formula (A-ii), and wherein R^{A3} is —CH₃, R^{A4} is —Cl, and R^{A5} is —CH₃, provided is a compound of formula:

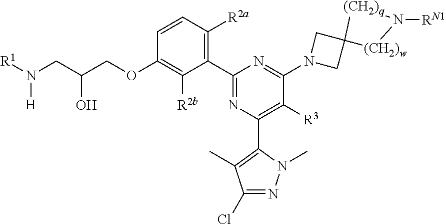

or a pharmaceutically acceptable salt thereof.

In certain embodiments, Ring B is of formula (xxvii), wherein Ring A is of formula (A-ii), and wherein R^{A3} is —CH₃, R^{A4} is hydrogen, and R^{A5} is —CH₃, provided is a compound of formula:

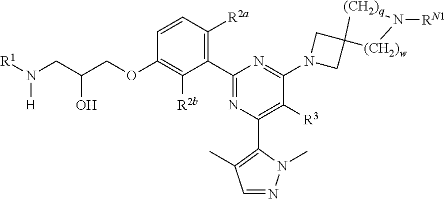

or a pharmaceutically acceptable salt thereof.

In certain embodiments, Ring B is of formula (xxvii), wherein Ring A is of formula (A-ii), and wherein R^{A3} is —CH₃, R^{A4} is hydrogen, and R^{A5} is —CH₂CH₃, provided is a compound of formula:

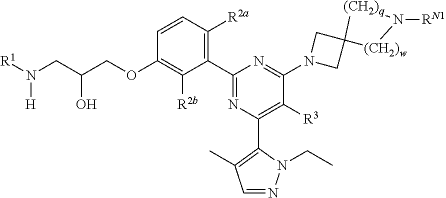

or a pharmaceutically acceptable salt thereof.

In certain embodiments, Ring B is of formula (xxvii), wherein Ring A is of formula (A-ii), and wherein R^{A3} is —Cl, R^{A4} is hydrogen, and R^{A5} is —CH₂CH₃, provided is a compound of formula:

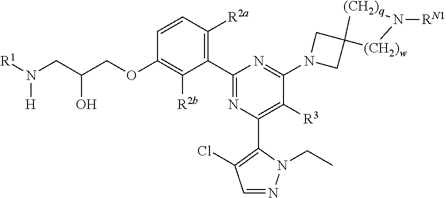

or a pharmaceutically acceptable salt thereof.

In certain embodiments, Ring B is of formula (xxvii), wherein Ring A is of formula (A-ii), and wherein R^{A3} is —Cl, R^{A4} is hydrogen, and R^{A5} is —CH₃, provided is a compound of formula:

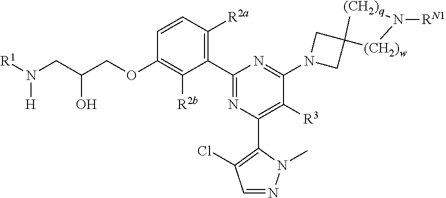

or a pharmaceutically acceptable salt thereof.

In certain embodiments, Ring B is of formula (xxvii), wherein Ring A is of formula (A-ii), and wherein R^{A3} is —Br, R^{A4} is hydrogen, and R^{A5} is —CH₃, provided is a compound of formula:

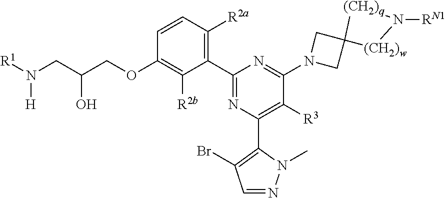

or a pharmaceutically acceptable salt thereof.

In certain embodiments, Ring B is of formula (xxvii), wherein Ring A is of formula (A-ii), and wherein R^{A3} is —CH₃, R^{A4} is —Br, and R^{A5} is —CH₃, provided is a compound of formula:

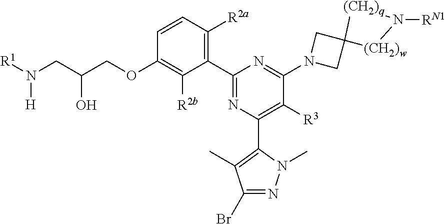

or a pharmaceutically acceptable salt thereof.

In certain embodiments, Ring B is of formula (xxvii), wherein Ring A is of formula (A-ii), and wherein R^{A3} is —CH₃, R^{A4} is —CN, and R^{A5} is —CH₃, provided is a compound of formula:

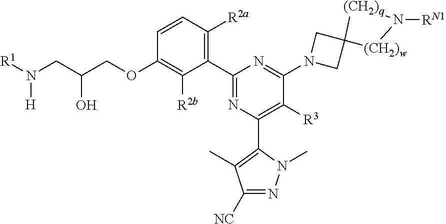

or a pharmaceutically acceptable salt thereof.

In certain embodiments, Ring B is of formula (xxviii), wherein Ring A is of formula (A-i), and wherein each of R^{A1} and R^{A2} is —CH₃, provided is a compound of formula:

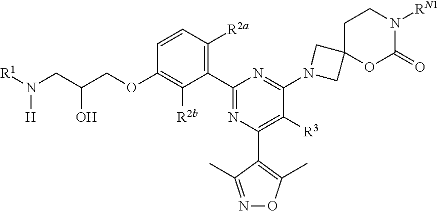

or a pharmaceutically acceptable salt thereof.

In certain embodiments, Ring B is of formula (xxix), wherein Ring A is of formula (A-i), and wherein each of R^{A1} and R^{A2} is —CH₃, provided is a compound of formula:

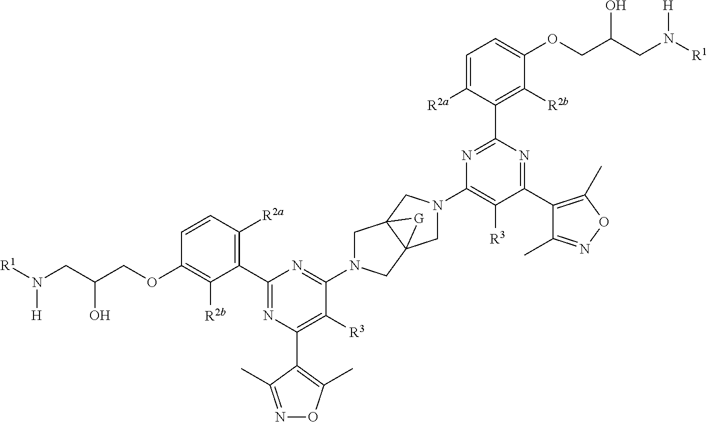

or a pharmaceutically acceptable salt thereof.

### (X) Exemplary Compounds

In certain embodiments, a compound of Formula (I) is selected from any one of the compounds provided in Table 1, and pharmaceutically acceptable salts thereof.

TABLE 1
Exemplary Compounds

| | | | *Cellular | LC-MS |
|---|---|---|---|---|
| | | *Biochem | IC₅₀ | m/z |
| # | Structure | IC₅₀ (uM) | (uM) | (M + H) |
| 1 | 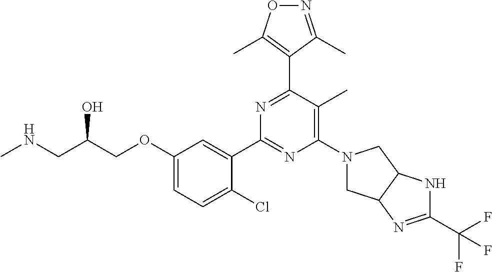 [SMILES: CNC[C@@H](O)COc1ccc(Cl)c(-c2nc(-c3c(C)noc3C)c(C)c(N3CC4N=C(C(F)(F)F)NC4C3)n2)c1] | 0.0061 | >20 | 580.2 |
| 2 | 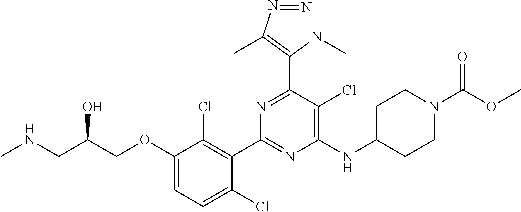 | 0.0401 | >20 | 615.2 |
| 3 | 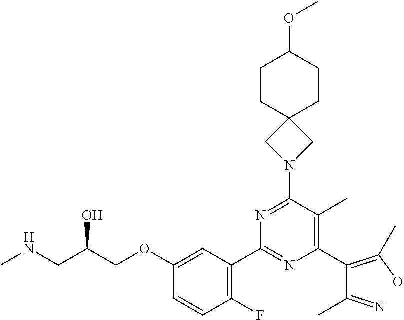 [SMILES: CNC[C@@H](O)COc1ccc(F)c(-c2nc(/C(C(C)=N)=C(\C)O)c(C)c(N3CC4(CCC(OC)CC4)C3)n2)c1] | 0.0067 | 0.105 | 539.8 |
| 4 | 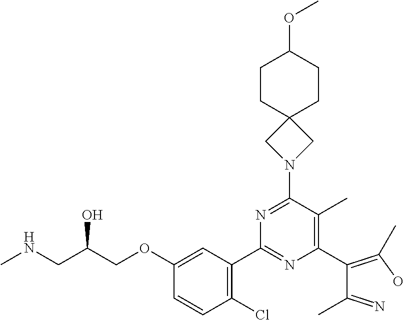 [SMILES: CNC[C@@H](O)COc1ccc(Cl)c(-c2nc(/C(C(C)=N)=C(\C)O)c(C)c(N3CC4(CCC(OC)CC4)C3)n2)c1] | 0.0078 | 0.051 | 555.8 |
| 5 | 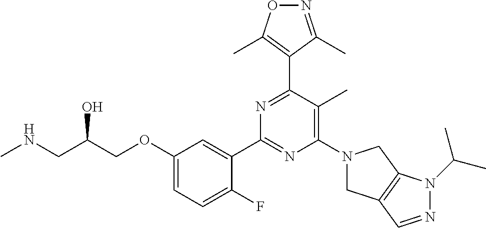 [SMILES: CNC[C@@H](O)COc1ccc(F)c(-c2nc(-c3c(C)noc3C)c(C)c(N3Cc4cnn(C(C)C)c4C3)n2)c1] | 0.0021 | 0.049 | 536.2 |
| 6 | 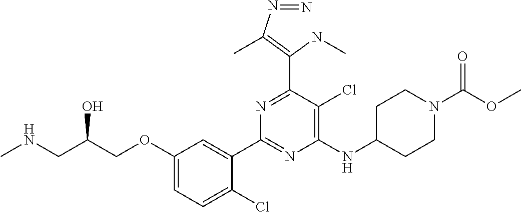 | 0.0047 | 0.455 | 579.2 |
| 7 | 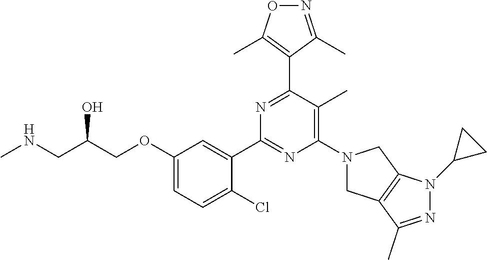 [SMILES: CNC[C@@H](O)COc1ccc(Cl)c(-c2nc(-c3c(C)noc3C)c(C)c(N3Cc4c(C)nn(C5CC5)c4C3)n2)c1] | 0.0031 | 0.105 | 564.2 |
| 8 | 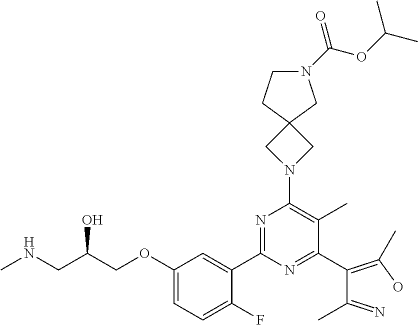 [SMILES: CNC[C@@H](O)COc1ccc(F)c(-c2nc(/C(C(C)=N)=C(\C)O)c(C)c(N3CC4(CCN(C(=O)OC(C)C)C4)C3)n2)c1] | 0.0033 | 0.103 | 583.3 |
| 9 | 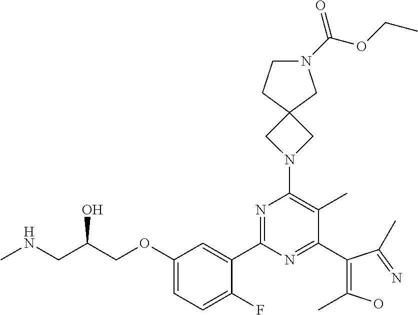 [SMILES: CCOC(=O)N1CCC2(C1)CN(c1nc(-c3cc(OC[C@H](O)CNC)ccc3F)nc(/C(C(C)=N)=C(\C)O)c1C)C2] | 0.0040 | 0.150 | 569.3 |
| 10 | 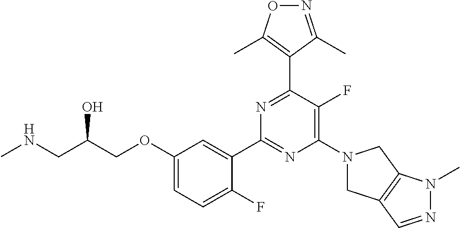 | 0.0023 | 0.126 | 512.3 |
| 11 | 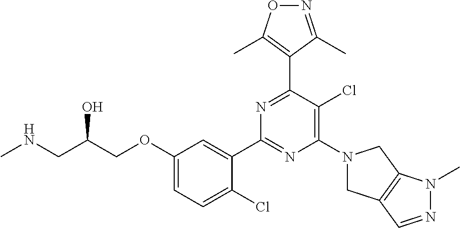 | 0.0607 | >20 | 544.3 |
| 12 | 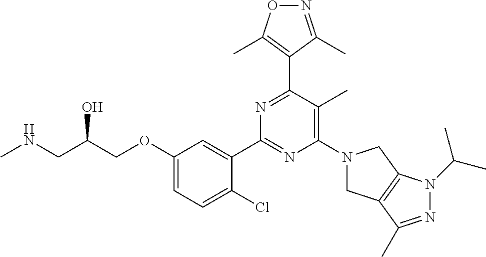 [SMILES: CNC[C@@H](O)COc1ccc(Cl)c(-c2nc(-c3c(C)noc3C)c(C)c(N3Cc4c(C)nn(C(C)C)c4C3)n2)c1] | 0.0041 | 0.081 | 566.2 |
| 13 | 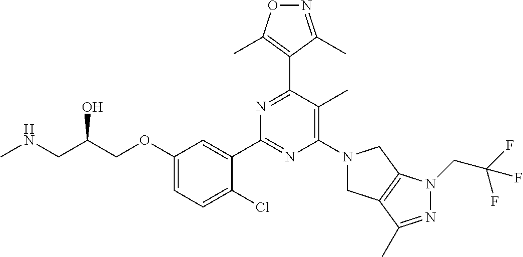 [SMILES: CNC[C@@H](O)COc1ccc(Cl)c(-c2nc(-c3c(C)noc3C)c(C)c(N3Cc4c(C)nn(CC(F)(F)F)c4C3)n2)c1] | 0.0036 | 0.074 | 606.2 |
| 14 | 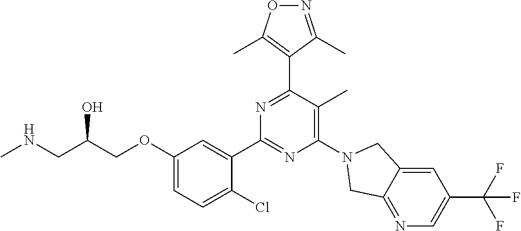 [SMILES: CNC[C@@H](O)COc1ccc(Cl)c(-c2nc(-c3c(C)noc3C)c(C)c(N3Cc4cc(C(F)(F)F)cnc4C3)n2)c1] | 0.0022 | 0.044 | 589.2 |
| 15 | 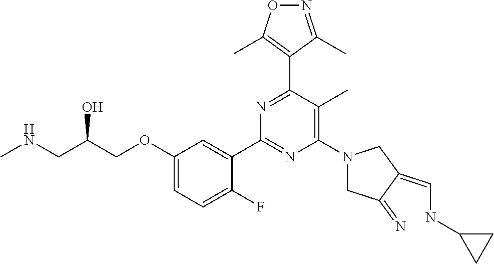 [SMILES: CNC[C@@H](O)COc1ccc(F)c(-c2nc(-c3c(C)noc3C)c(C)c(N3CC(=N)/C(=C\NC4CC4)C3)n2)c1] | 0.0019 | 0.140 | 533.8 |
| 16 | 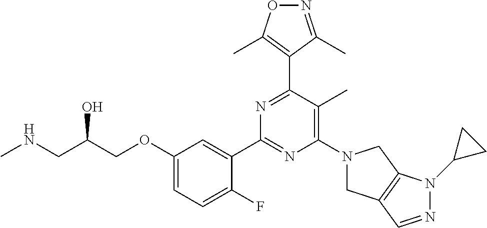 [SMILES: CNC[C@@H](O)COc1ccc(F)c(-c2nc(-c3c(C)noc3C)c(C)c(N3Cc4cnn(C5CC5)c4C3)n2)c1] | 0.0021 | 0.056 | 533.8 |
| 17 | 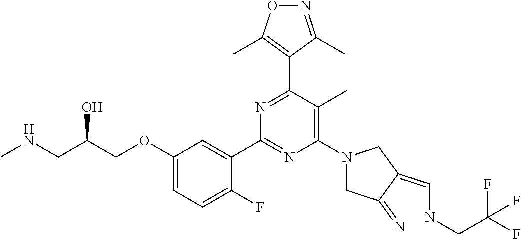 [SMILES: CNC[C@@H](O)COc1ccc(F)c(-c2nc(-c3c(C)noc3C)c(C)c(N3CC(=N)/C(=C\NCC(F)(F)F)C3)n2)c1] | 0.0024 | 0.162 | 575.7 |
| 18 | 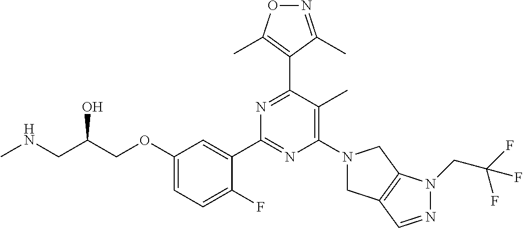 [SMILES: CNC[C@@H](O)COc1ccc(F)c(-c2nc(-c3c(C)noc3C)c(C)c(N3Cc4cnn(CC(F)(F)F)c4C3)n2)c1] | 0.0031 | 0.069 | 575.8 |
| 19 | 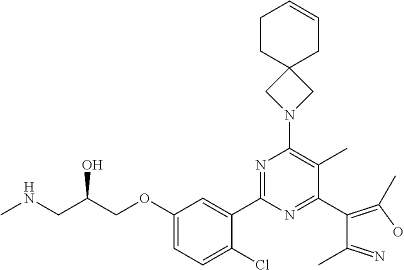 [SMILES: CNC[C@@H](O)COc1ccc(Cl)c(-c2nc(/C(C(C)=N)=C(\C)O)c(C)c(N3CC4(CC=CCC4)C3)n2)c1] | 0.0041 | 0.195 | 524.2 |
| 20 | 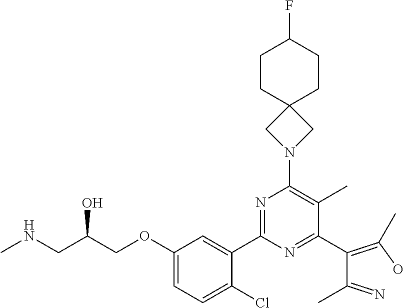 [SMILES: CNC[C@@H](O)COc1ccc(Cl)c(-c2nc(/C(C(C)=N)=C(\C)O)c(C)c(N3CC4(CCC(F)CC4)C3)n2)c1] | 0.0074 | 0.125 | 544.3 |
| 21 | 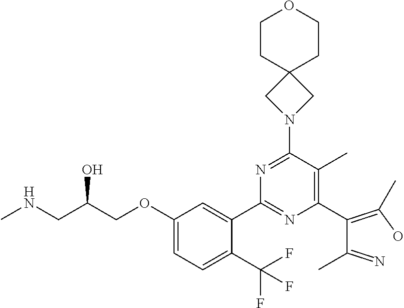 [SMILES: CNC[C@@H](O)COc1ccc(C(F)(F)F)c(-c2nc(/C(C(C)=N)=C(\C)O)c(C)c(N3CC4(CCOCC4)C3)n2)c1] | 0.0166 | 0.179 | 562.1 |
| 22 | 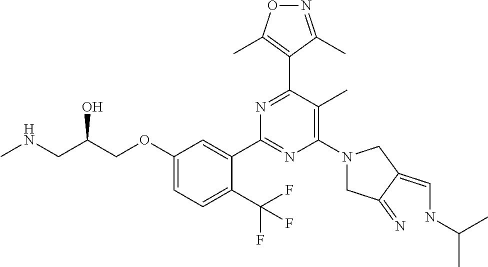 [SMILES: CNC[C@@H](O)COc1ccc(C(F)(F)F)c(-c2nc(-c3c(C)noc3C)c(C)c(N3CC(=N)/C(=C\NC(C)C)C3)n2)c1] | 0.0063 | 0.181 | 585.7 |
| 23 | 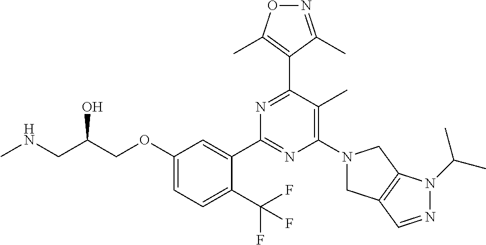 [SMILES: CNC[C@@H](O)COc1ccc(C(F)(F)F)c(-c2nc(-c3c(C)noc3C)c(C)c(N3Cc4cnn(C(C)C)c4C3)n2)c1] | 0.0047 | 0.111 | 585.7 |
| 24 | 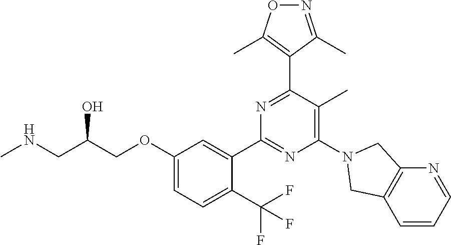 [SMILES: CNC[C@@H](O)COc1ccc(C(F)(F)F)c(-c2nc(-c3c(C)noc3C)c(C)c(N3Cc4cccnc4C3)n2)c1] | 0.0054 | 0.188 | 555.1 |
| 25 | 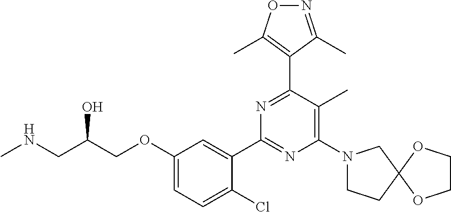 [SMILES: CNC[C@@H](O)COc1ccc(Cl)c(-c2nc(-c3c(C)noc3C)c(C)c(N3CCC4(C3)OCCO4)n2)c1] | 0.0056 | 0.452 | 530 |
| 26 | 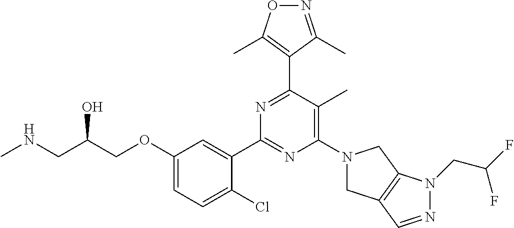 [SMILES: CNC[C@@H](O)COc1ccc(Cl)c(-c2nc(-c3c(C)noc3C)c(C)c(N3Cc4cnn(CC(F)F)c4C3)n2)c1] | 0.0034 | 0.066 | 574.2 |
| 27 | 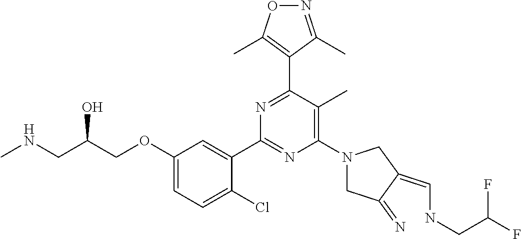 [SMILES: CNC[C@@H](O)COc1ccc(Cl)c(-c2nc(-c3c(C)noc3C)c(C)c(N3CC(=N)/C(=C\NCC(F)F)C3)n2)c1] | 0.0037 | 0.115 | 574.2 |
| 28 | 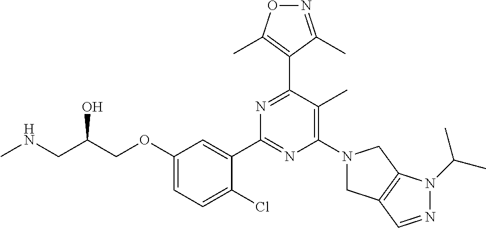 [SMILES: CNC[C@@H](O)COc1ccc(Cl)c(-c2nc(-c3c(C)noc3C)c(C)c(N3Cc4cnn(C(C)C)c4C3)n2)c1] | 0.0032 | 0.041 | 551.8 |
| 29 | 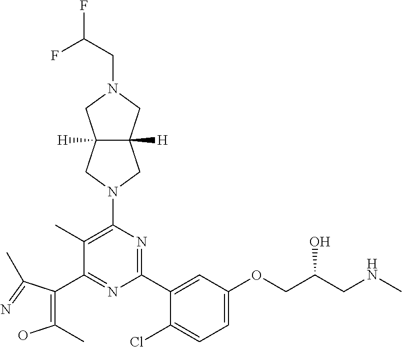 [SMILES: CNC[C@@H](O)COc1ccc(Cl)c(-c2nc(/C(C(C)=N)=C(\C)O)c(C)c(N3C[C@H]4CN(CC(F)F)C[C@@H]4C3)n2)c1] | 0.0102 | 0.263 | 577.3 |
| 30 | 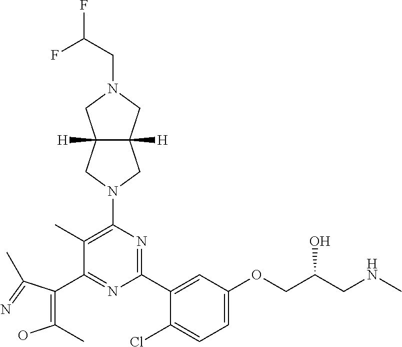 [SMILES: CNC[C@@H](O)COc1ccc(Cl)c(-c2nc(/C(C(C)=N)=C(\C)O)c(C)c(N3C[C@H]4CN(CC(F)F)C[C@H]4C3)n2)c1] | 0.0070 | 0.166 | 577.3 |
| 31 | 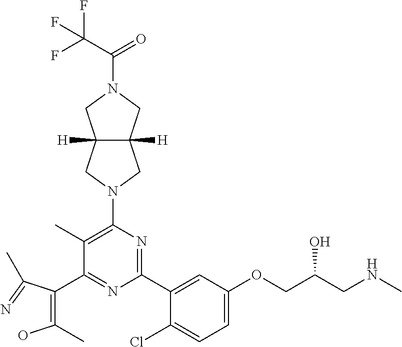 [SMILES: CNC[C@@H](O)COc1ccc(Cl)c(-c2nc(/C(C(C)=N)=C(\C)O)c(C)c(N3C[C@H]4CN(C(=O)C(F)(F)F)C[C@H]4C3)n2)c1] | 0.0014 | 2.154 | 609.2 |
| 32 | 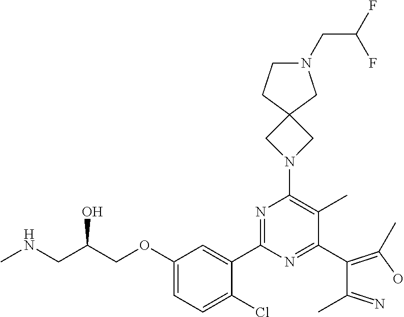 [SMILES: CNC[C@@H](O)COc1ccc(Cl)c(-c2nc(/C(C(C)=N)=C(\C)O)c(C)c(N3CC4(CCN(CC(F)F)C4)C3)n2)c1] | 0.0070 | 0.053 | 577 |
| 33 | 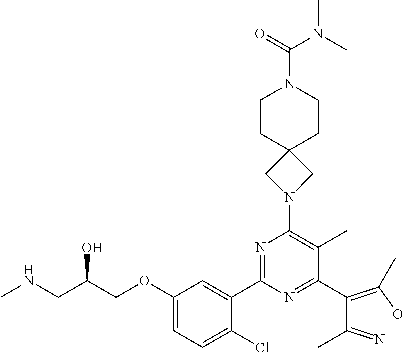 [SMILES: CNC[C@@H](O)COc1ccc(Cl)c(-c2nc(/C(C(C)=N)=C(\C)O)c(C)c(N3CC4(CCN(C(=O)N(C)C)CC4)C3)n2)c1] | 0.0072 | 0.083 | 598.3 |
| 34 | 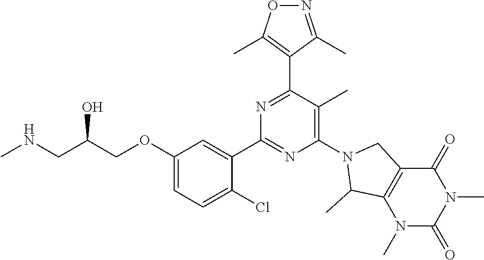 | 0.0051 | 0.120 | 596.2 |
| 35 | 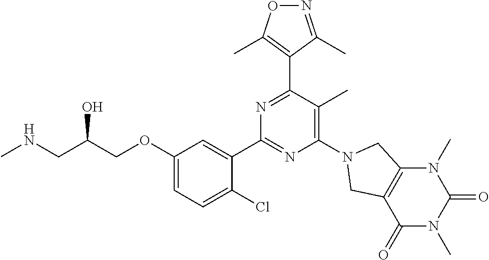 [SMILES: CNC[C@@H](O)COc1ccc(Cl)c(-c2nc(-c3c(C)noc3C)c(C)c(N3Cc4c(n(C)c(=O)n(C)c4=O)C3)n2)c1] | 0.0037 | 0.412 | 582.2 |
| 36 | 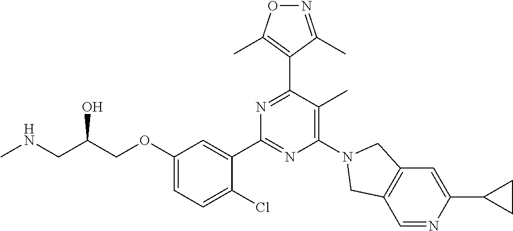 [SMILES: CNC[C@@H](O)COc1ccc(Cl)c(-c2nc(-c3c(C)noc3C)c(C)c(N3Cc4cnc(C5CC5)cc4C3)n2)c1] | 0.0024 | 0.049 | 560.8 |
| 37 | 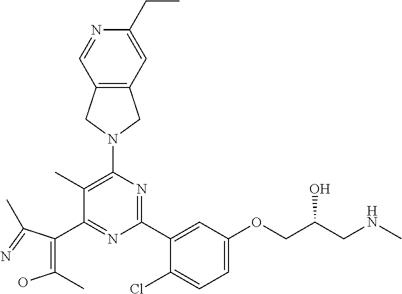 [SMILES: CCc1cc2c(cn1)CN(c1nc(-c3cc(OC[C@H](O)CNC)ccc3Cl)nc(/C(C(C)=N)=C(\C)O)c1C)C2] | 0.0026 | 0.037 | 548.8 |
| 38 | 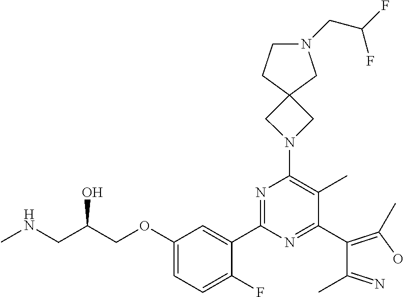 [SMILES: CNC[C@@H](O)COc1ccc(F)c(-c2nc(/C(C(C)=N)=C(\C)O)c(C)c(N3CC4(CCN(CC(F)F)C4)C3)n2)c1] | 0.0057 | 0.076 | 561.1 |
| 39 | 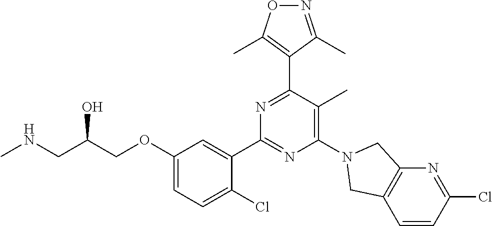 [SMILES: CNC[C@@H](O)COc1ccc(Cl)c(-c2nc(-c3c(C)noc3C)c(C)c(N3Cc4ccc(Cl)nc4C3)n2)c1] | 0.0020 | 0.032 | 555.2 |
| 40 | 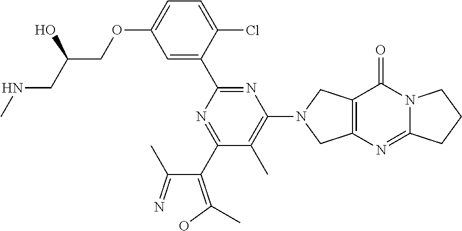 [SMILES: CNC[C@@H](O)COc1ccc(Cl)c(-c2nc(/C(C(C)=N)=C(\C)O)c(C)c(N3Cc4nc5n(c(=O)c4C3)CCC5)n2)c1] | 0.0028 | 0.391 | 578.2 |
| 41 | 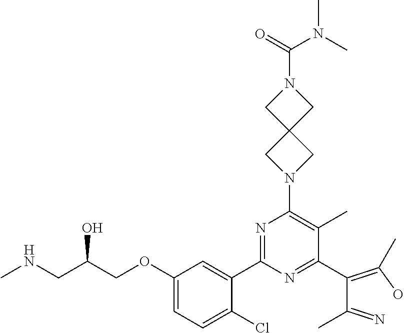 [SMILES: CNC[C@@H](O)COc1ccc(Cl)c(-c2nc(/C(C(C)=N)=C(\C)O)c(C)c(N3CC4(CN(C(=O)N(C)C)C4)C3)n2)c1] | 0.0087 | 0.546 | 570.2 |
| 42 | 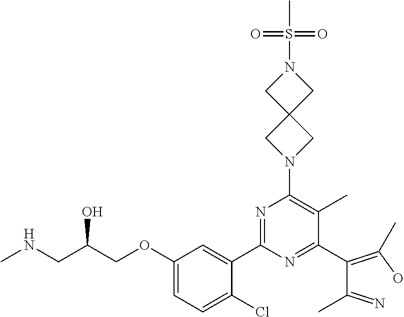 [SMILES: CNC[C@@H](O)COc1ccc(Cl)c(-c2nc(/C(C(C)=N)=C(\C)O)c(C)c(N3CC4(C3)CN(S(C)(=O)=O)C4)n2)c1] | 0.0104 | 0.406 | 577.2 |
| 43 | 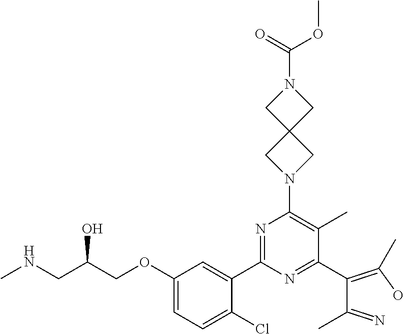 [SMILES: CNC[C@@H](O)COc1ccc(Cl)c(-c2nc(/C(C(C)=N)=C(\C)O)c(C)c(N3CC4(CN(C(=O)OC)C4)C3)n2)c1] | 0.0100 | 0.084 | 557.2 |
| 44 | 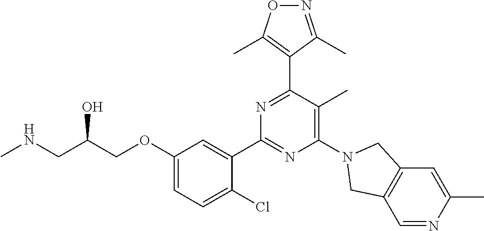 | 0.0029 | 0.027 | 534.8 |
| 45 | 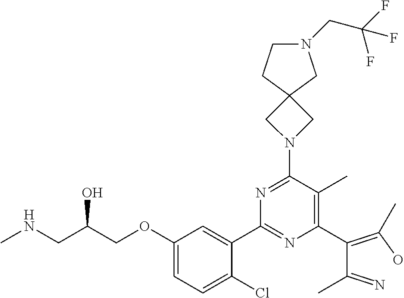 [SMILES: CNC[C@@H](O)COc1ccc(Cl)c(-c2nc(/C(C(C)=N)=C(\C)O)c(C)c(N3CC4(CCN(CC(F)(F)F)C4)C3)n2)c1] | 0.0119 | 0.040 | 595.1 |
| 46 | 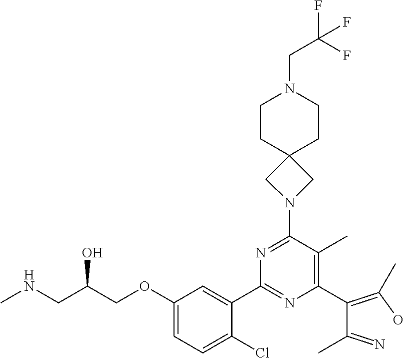 [SMILES: CNC[C@@H](O)COc1ccc(Cl)c(-c2nc(/C(C(C)=N)=C(\C)O)c(C)c(N3CC4(CCN(CC(F)(F)F)CC4)C3)n2)c1] | 0.0174 | 0.035 | 609.1 |
| 47 | 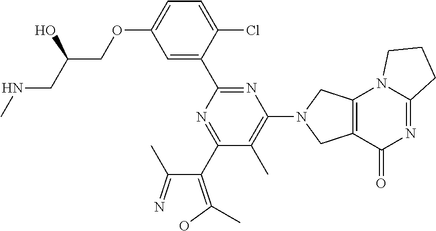 [SMILES: CNC[C@@H](O)COc1ccc(Cl)c(-c2nc(/C(C(C)=N)=C(\C)O)c(C)c(N3Cc4c(n5c(nc4=O)CCC5)C3)n2)c1] | 0.0063 | >20 | 578.2 |
| 48 | 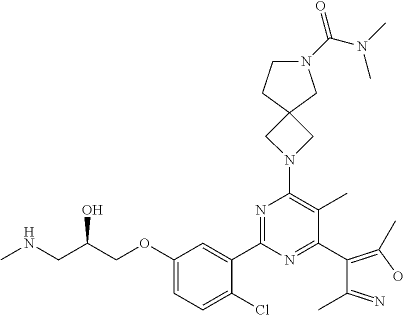 [SMILES: CNC[C@@H](O)COc1ccc(Cl)c(-c2nc(/C(C(C)=N)=C(\C)O)c(C)c(N3CC4(CCN(C(=O)N(C)C)C4)C3)n2)c1] | 0.0127 | 0.238 | 584.2 |
| 49 | 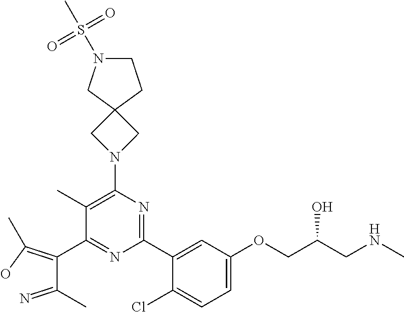 [SMILES: CNC[C@@H](O)COc1ccc(Cl)c(-c2nc(/C(C(C)=N)=C(\C)O)c(C)c(N3CC4(CCN(S(C)(=O)=O)C4)C3)n2)c1] | 0.0077 | 0.399 | 591.1 |
| 50 | 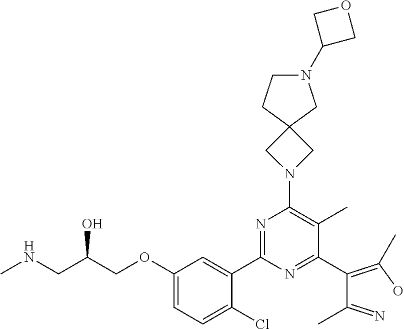 [SMILES: CNC[C@@H](O)COc1ccc(Cl)c(-c2nc(/C(C(C)=N)=C(\C)O)c(C)c(N3CC4(CCN(C5COC5)C4)C3)n2)c1] | 0.0088 | 0.155 | 569.2 |
| 51 | 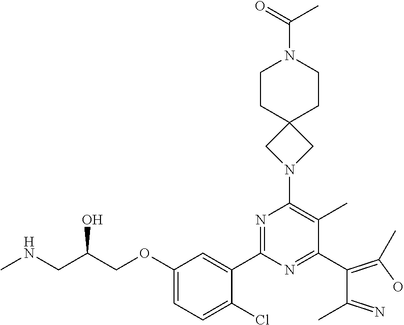 [SMILES: CNC[C@@H](O)COc1ccc(Cl)c(-c2nc(/C(C(C)=N)=C(\C)O)c(C)c(N3CC4(CCN(C(C)=O)CC4)C3)n2)c1] | 0.0108 | 0.356 | 569.3 |
| 52 | 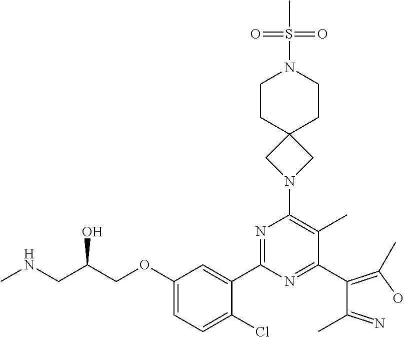 [SMILES: CNC[C@@H](O)COc1ccc(Cl)c(-c2nc(/C(C(C)=N)=C(\C)O)c(C)c(N3CC4(CCN(S(C)(=O)=O)CC4)C3)n2)c1] | 0.0073 | 0.230 | 605.2 |
| 53 | 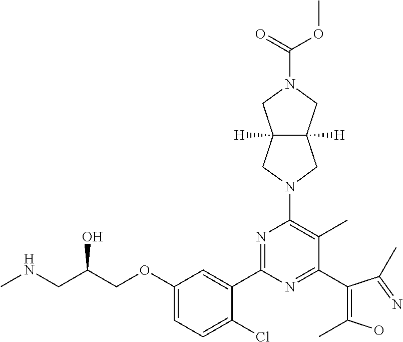 [SMILES: CNC[C@@H](O)COc1ccc(Cl)c(-c2nc(/C(C(C)=N)=C(\C)O)c(C)c(N3C[C@H]4CN(C(=O)OC)C[C@H]4C3)n2)c1] | 0.0053 | 0.198 | 571.1 |
| 54 | 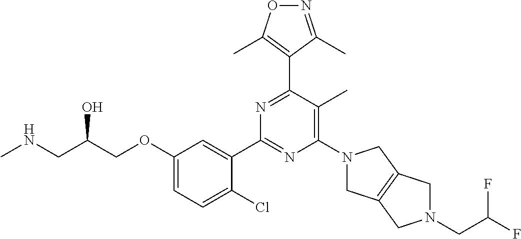 [SMILES: CNC[C@@H](O)COc1ccc(Cl)c(-c2nc(-c3c(C)noc3C)c(C)c(N3CC4=C(CN(CC(F)F)C4)C3)n2)c1] | 0.0044 | 0.045 | 575 |
| 55 | 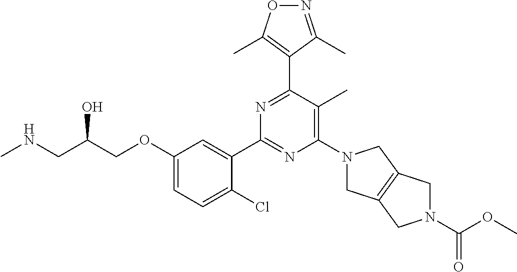 | 0.0036 | 0.013 | 569.1 |
| 56 | 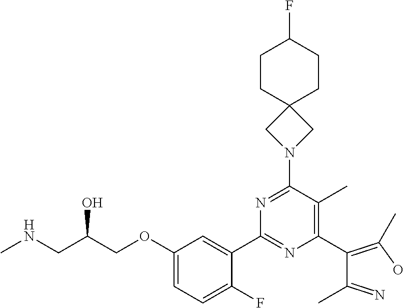 [SMILES: CNC[C@@H](O)COc1ccc(F)c(-c2nc(/C(C(C)=N)=C(\C)O)c(C)c(N3CC4(CCC(F)CC4)C3)n2)c1] | 0.0038 | 0.113 | 528.1 |
| 57 | 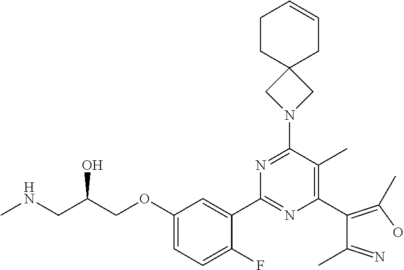 [SMILES: CNC[C@@H](O)COc1ccc(F)c(-c2nc(/C(C(C)=N)=C(\C)O)c(C)c(N3CC4(CC=CCC4)C3)n2)c1] | 0.0034 | 0.276 | 508.1 |
| 58 | 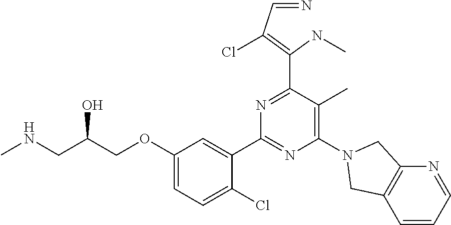 [SMILES: CNC[C@@H](O)COc1ccc(Cl)c(-c2nc(/C(NC)=C(\Cl)C=N)c(C)c(N3Cc4cccnc4C3)n2)c1] | 0.0014 | 0.073 | 540.2 |
| 59 | 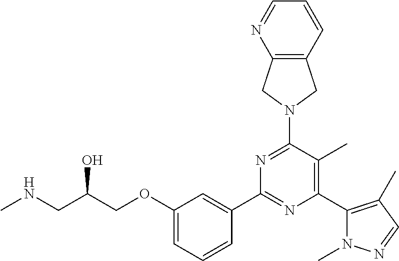 [SMILES: CNC[C@@H](O)COc1cccc(-c2nc(-c3c(C)cnn3C)c(C)c(N3Cc4cccnc4C3)n2)c1] | 0.0018 | 0.292 | 486.1 |
| 60 | 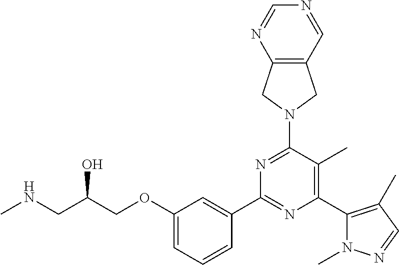 [SMILES: CNC[C@@H](O)COc1cccc(-c2nc(-c3c(C)cnn3C)c(C)c(N3Cc4cncnc4C3)n2)c1] | 0.0039 | 0.389 | 487.1 |
| 61 | 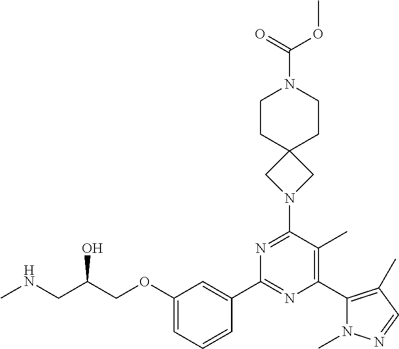 [SMILES: CNC[C@@H](O)COc1cccc(-c2nc(-c3c(C)cnn3C)c(C)c(N3CC4(CCN(C(=O)OC)CC4)C3)n2)c1] | 0.0137 | 0.874 | 550.2 |
| 62 | 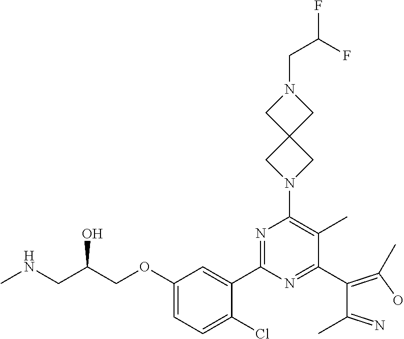 [SMILES: CNC[C@@H](O)COc1ccc(Cl)c(-c2nc(/C(C(C)=N)=C(\C)O)c(C)c(N3CC4(CN(CC(F)F)C4)C3)n2)c1] | 0.0647 | 0.526 | 563.2 |
| 63 | 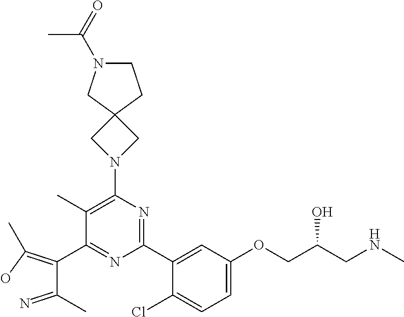 [SMILES: CNC[C@@H](O)COc1ccc(Cl)c(-c2nc(/C(C(C)=N)=C(\C)O)c(C)c(N3CC4(CCN(C(C)=O)C4)C3)n2)c1] | 0.0110 | 2.064 | 555.1 |
| 64 | 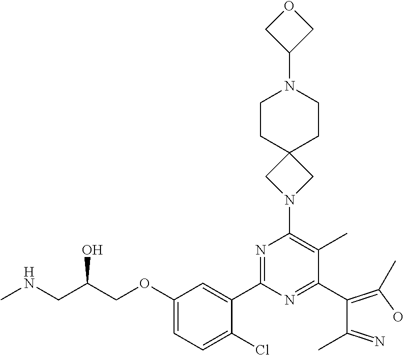 [SMILES: CNC[C@@H](O)COc1ccc(Cl)c(-c2nc(/C(C(C)=N)=C(\C)O)c(C)c(N3CC4(CCN(C5COC5)CC4)C3)n2)c1] | 0.0110 | 0.111 | 583.1 |
| 65 | 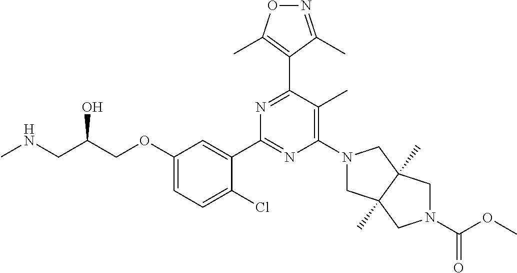 | 0.0078 | 0.234 | 599.1 |
| 66 | 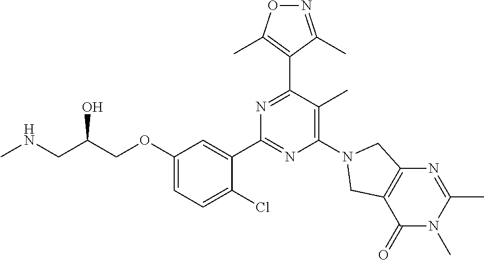 | 0.0037 | 0.261 | 566.2 |
| 67 | 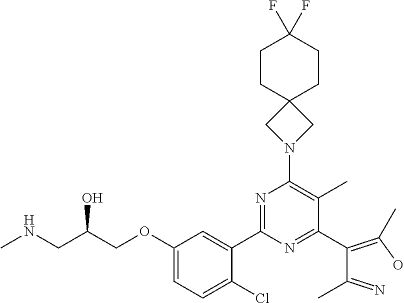 [SMILES: CNC[C@@H](O)COc1ccc(Cl)c(-c2nc(/C(C(C)=N)=C(\C)O)c(C)c(N3CC4(CCC(F)(F)CC4)C3)n2)c1] | 0.0050 | 0.071 | 562.1 |
| 68 | 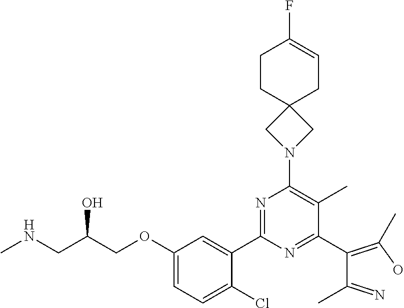 [SMILES: CNC[C@@H](O)COc1ccc(Cl)c(-c2nc(/C(C(C)=N)=C(\C)O)c(C)c(N3CC4(CC=C(F)CC4)C3)n2)c1] | 0.0039 | 0.144 | 542.1 |
| 69 | 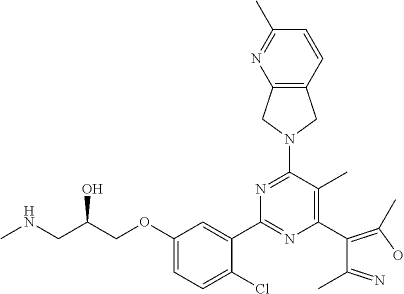 [SMILES: CNC[C@@H](O)COc1ccc(Cl)c(-c2nc(/C(C(C)=N)=C(\C)O)c(C)c(N3Cc4ccc(C)nc4C3)n2)c1] | 0.0012 | 0.022 | 535.1 |
| 70 | 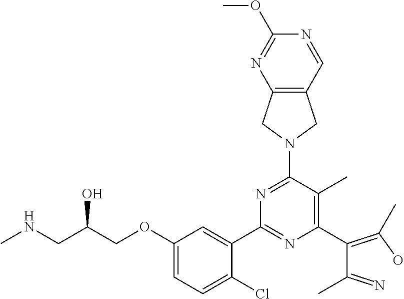 [SMILES: CNC[C@@H](O)COc1ccc(Cl)c(-c2nc(/C(C(C)=N)=C(\C)O)c(C)c(N3Cc4cnc(OC)nc4C3)n2)c1] | 0.0032 | 0.019 | 552.2 |
| 71 | 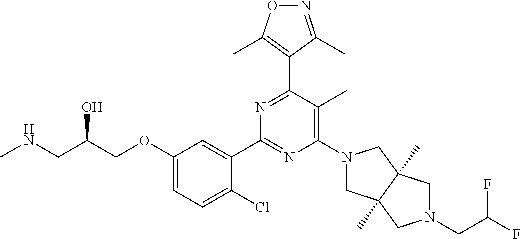 [SMILES: CNC[C@@H](O)COc1ccc(Cl)c(-c2nc(-c3c(C)noc3C)c(C)c(N3C[C@]4(C)CN(CC(F)F)C[C@]4(C)C3)n2)c1] | 0.0056 | 0.221 | 605.2 |
| 72 | 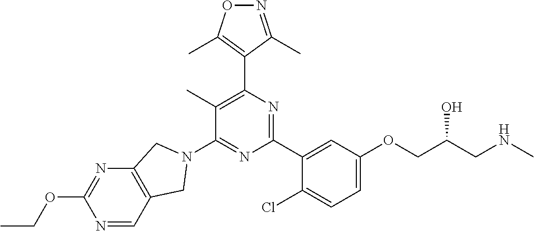 [SMILES: CCOc1ncc2c(n1)CN(c1nc(-c3cc(OC[C@H](O)CNC)ccc3Cl)nc(-c3c(C)noc3C)c1C)C2] | 0.0029 | 0.012 | 566.2 |
| 73 | 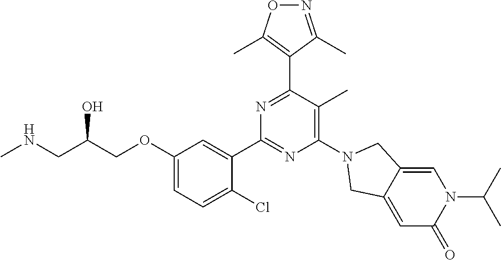 [SMILES: CNC[C@@H](O)COc1ccc(Cl)c(-c2nc(-c3c(C)noc3C)c(C)c(N3Cc4cc(=O)n(C(C)C)cc4C3)n2)c1] | 0.0061 | 0.422 | 578.8 |
| 74 | 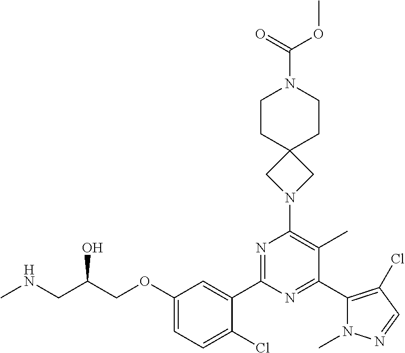 [SMILES: CNC[C@@H](O)COc1ccc(Cl)c(-c2nc(-c3c(Cl)cnn3C)c(C)c(N3CC4(CCN(C(=O)OC)CC4)C3)n2)c1] | 0.0097 | 0.207 | 604.2 |
| 75 | 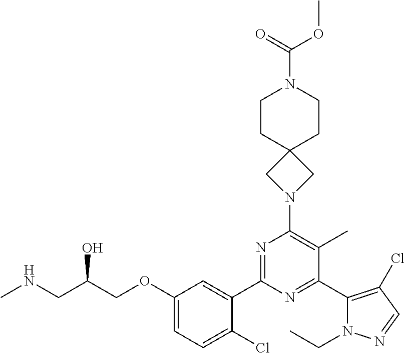 [SMILES: CCn1ncc(Cl)c1-c1nc(-c2cc(OC[C@H](O)CNC)ccc2Cl)nc(N2CC3(CCN(C(=O)OC)CC3)C2)c1C] | 0.0125 | 0.396 | 618 |
| 76 | 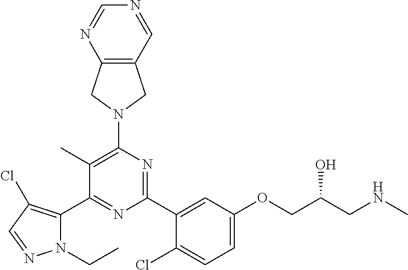 [SMILES: CCn1ncc(Cl)c1-c1nc(-c2cc(OC[C@H](O)CNC)ccc2Cl)nc(N2Cc3cncnc3C2)c1C] | 0.0048 | 0.268 | 555 |
| 77 | 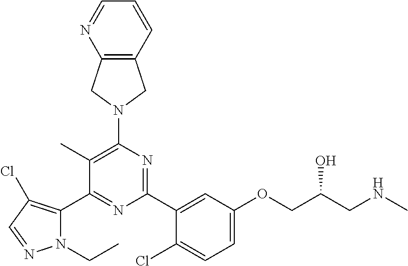 [SMILES: CCn1ncc(Cl)c1-c1nc(-c2cc(OC[C@H](O)CNC)ccc2Cl)nc(N2Cc3cccnc3C2)c1C] | 0.0037 | 0.293 | 554 |
| 78 | 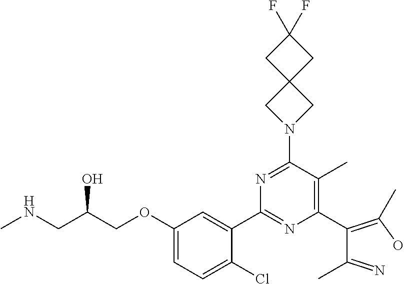 [SMILES: CNC[C@@H](O)COc1ccc(Cl)c(-c2nc(/C(C(C)=N)=C(\C)O)c(C)c(N3CC4(C3)CC(F)(F)C4)n2)c1] | 0.0056 | 0.112 | 534 |
| 79 | 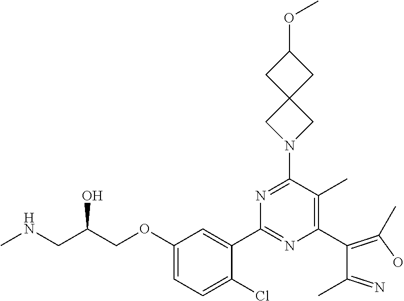 [SMILES: CNC[C@@H](O)COc1ccc(Cl)c(-c2nc(/C(C(C)=N)=C(\C)O)c(C)c(N3CC4(CC(OC)C4)C3)n2)c1] | 0.0093 | 0.060 | 528 |
| 80 | 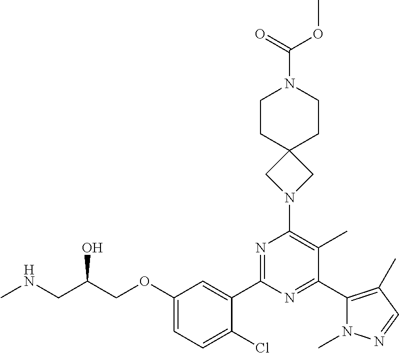 [SMILES: CNC[C@@H](O)COc1ccc(Cl)c(-c2nc(-c3c(C)cnn3C)c(C)c(N3CC4(CCN(C(=O)OC)CC4)C3)n2)c1] | 0.0088 | 0.182 | 584.2 |
| 81 | 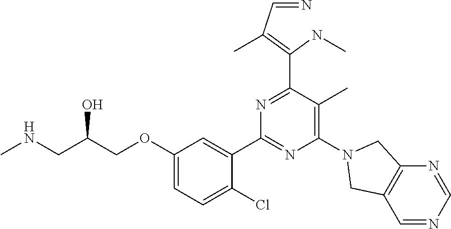 [SMILES: CNC[C@@H](O)COc1ccc(Cl)c(-c2nc(/C(NC)=C(\C)C=N)c(C)c(N3Cc4cncnc4C3)n2)c1] | 0.0038 | 0.112 | 521.3 |
| 82 | 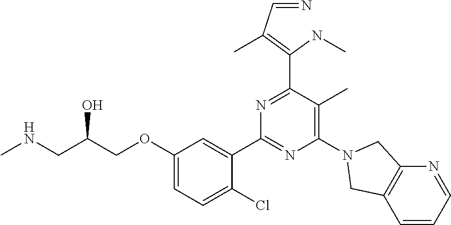 [SMILES: CNC[C@@H](O)COc1ccc(Cl)c(-c2nc(/C(NC)=C(\C)C=N)c(C)c(N3Cc4cccnc4C3)n2)c1] | 0.0021 | 0.056 | 520.2 |
| 83 | 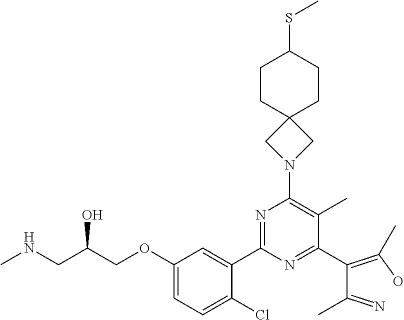 [SMILES: CNC[C@@H](O)COc1ccc(Cl)c(-c2nc(/C(C(C)=N)=C(\C)O)c(C)c(N3CC4(CCC(SC)CC4)C3)n2)c1] | 0.0033 | 0.074 | 572.2 |
| 84 | 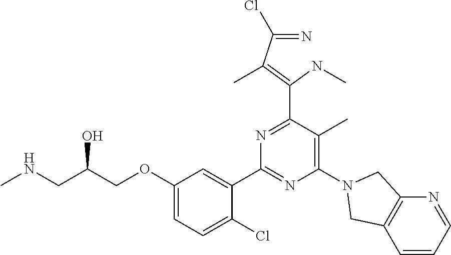 [SMILES: CNC[C@@H](O)COc1ccc(Cl)c(-c2nc(/C(NC)=C(\C)C(=N)Cl)c(C)c(N3Cc4cccnc4C3)n2)c1] | 0.0016 | 0.028 | 554.2 |
| 85 | 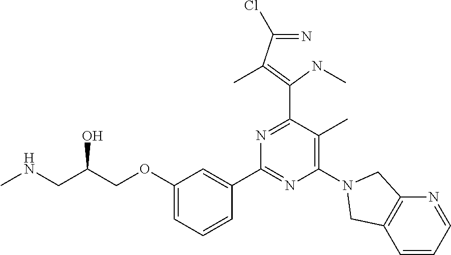 [SMILES: CNC[C@@H](O)COc1cccc(-c2nc(/C(NC)=C(\C)C(=N)Cl)c(C)c(N3Cc4cccnc4C3)n2)c1] | 0.0011 | 0.083 | 520.2 |
| 86 | 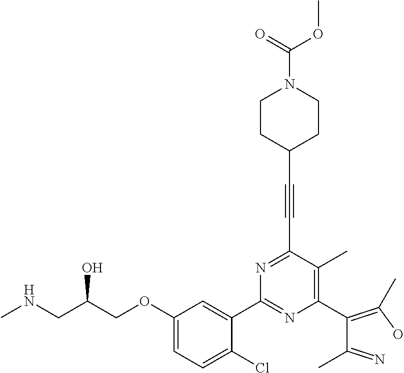 [SMILES: CNC[C@@H](O)COc1ccc(Cl)c(-c2nc(C#CC3CCN(C(=O)OC)CC3)c(C)c(/C(C(C)=N)=C(\C)O)n2)c1] | 0.0970 | 0.283 | 568.2 |
| 87 | 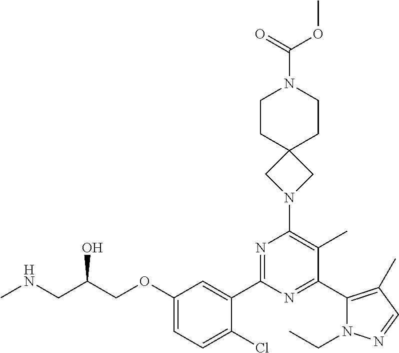 [SMILES: CCn1ncc(C)c1-c1nc(-c2cc(OC[C@H](O)CNC)ccc2Cl)nc(N2CC3(CCN(C(=O)OC)CC3)C2)c1C] | 0.0090 | 0.205 | 598.2 |
| 88 | 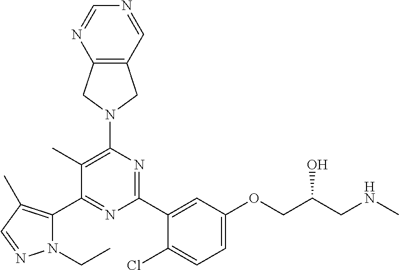 [SMILES: CCn1ncc(C)c1-c1nc(-c2cc(OC[C@H](O)CNC)ccc2Cl)nc(N2Cc3cncnc3C2)c1C] | 0.0046 | 0.222 | 535.2 |
| 89 | 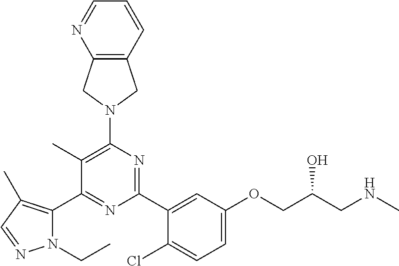 [SMILES: CCn1ncc(C)c1-c1nc(-c2cc(OC[C@H](O)CNC)ccc2Cl)nc(N2Cc3cccnc3C2)c1C] | 0.0037 | 0.165 | 534.2 |
| 90 | 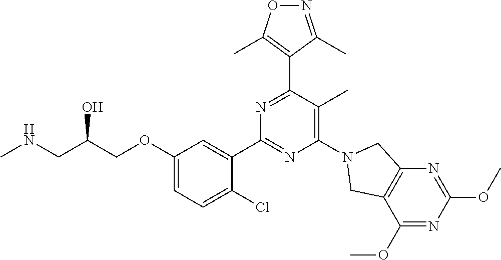 | 0.0022 | 0.032 | 582.1 |
| 91 | 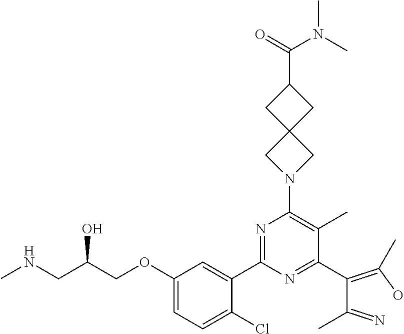 [SMILES: CNC[C@@H](O)COc1ccc(Cl)c(-c2nc(/C(C(C)=N)=C(\C)O)c(C)c(N3CC4(CC(C(=O)N(C)C)C4)C3)n2)c1] | 0.0055 | 0.092 | 569.1 |
| 92 | 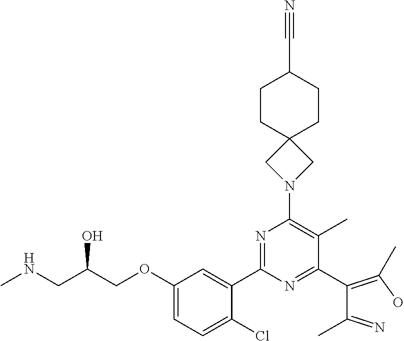 [SMILES: CNC[C@@H](O)COc1ccc(Cl)c(-c2nc(/C(C(C)=N)=C(\C)O)c(C)c(N3CC4(CCC(C#N)CC4)C3)n2)c1] | 0.0052 | 0.064 | 551.2 |
| 93 | 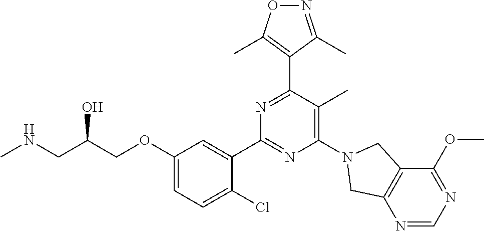 | 0.0023 | 0.033 | 552.1 |
| 94 | 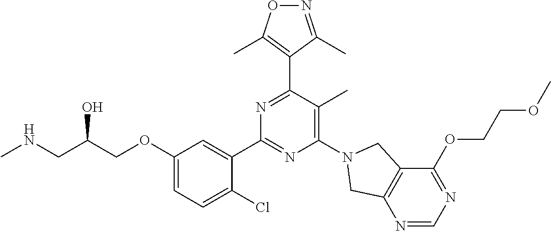 | 0.0023 | 0.044 | 596.3 |
| 95 | 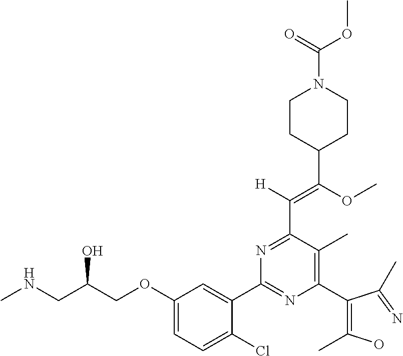 [SMILES: CNC[C@@H](O)COc1ccc(Cl)c(-c2nc(/C=C(\OC)C3CCN(C(=O)OC)CC3)c(C)c(/C(C(C)=N)=C(\C)O)n2)c1] | 0.0112 | 0.995 | 600.1 |
| 96 | 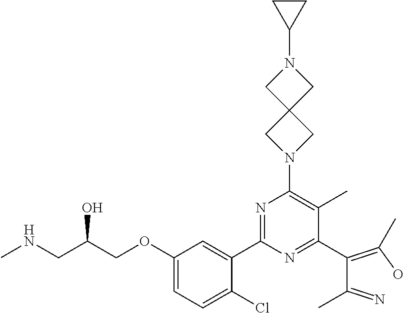 [SMILES: CNC[C@@H](O)COc1ccc(Cl)c(-c2nc(/C(C(C)=N)=C(\C)O)c(C)c(N3CC4(C3)CN(C3CC3)C4)n2)c1] | 0.0021 | 0.054 | 539.1 |
| 97 | 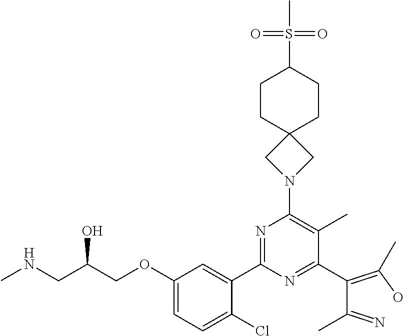 [SMILES: CNC[C@@H](O)COc1ccc(Cl)c(-c2nc(/C(C(C)=N)=C(\C)O)c(C)c(N3CC4(CCC(S(C)(=O)=O)CC4)C3)n2)c1] | 0.0109 | 0.394 | 604.1 |
| 98 | 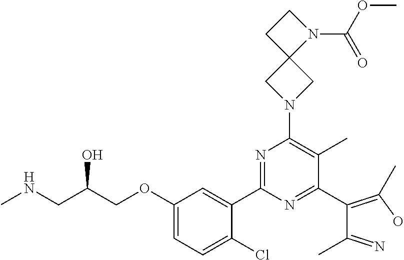 [SMILES: CNC[C@@H](O)COc1ccc(Cl)c(-c2nc(/C(C(C)=N)=C(\C)O)c(C)c(N3CC4(CCN4C(=O)OC)C3)n2)c1] | 0.0084 | 0.099 | 557.2 |
| 99 | 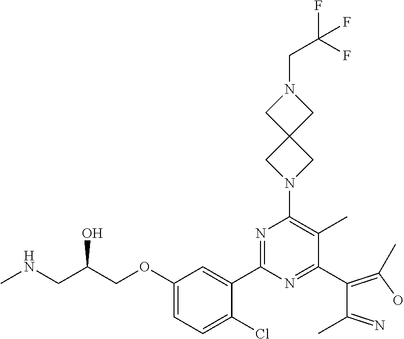 [SMILES: CNC[C@@H](O)COc1ccc(Cl)c(-c2nc(/C(C(C)=N)=C(\C)O)c(C)c(N3CC4(CN(CC(F)(F)F)C4)C3)n2)c1] | 0.0110 | 0.039 | 581.1 |
| 100 | 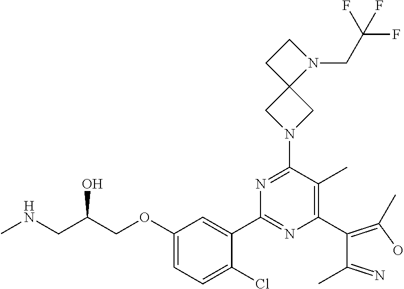 [SMILES: CNC[C@@H](O)COc1ccc(Cl)c(-c2nc(/C(C(C)=N)=C(\C)O)c(C)c(N3CC4(CCN4CC(F)(F)F)C3)n2)c1] | 0.0113 | 0.068 | 581.1 |
| 101 | 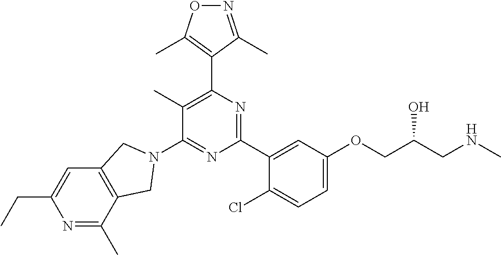 [SMILES: CCc1cc2c(c(C)n1)CN(c1nc(-c3cc(OC[C@H](O)CNC)ccc3Cl)nc(-c3c(C)noc3C)c1C)C2] | 0.0044 | 0.038 | 563.1 |
| 102 | 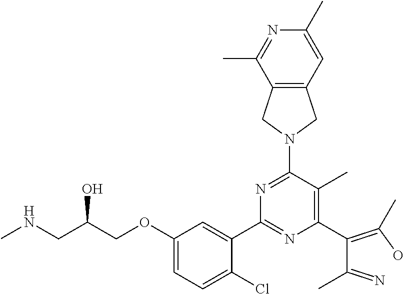 [SMILES: CNC[C@@H](O)COc1ccc(Cl)c(-c2nc(/C(C(C)=N)=C(\C)O)c(C)c(N3Cc4cc(C)nc(C)c4C3)n2)c1] | 0.0045 | 0.027 | 549.1 |
| 103 | 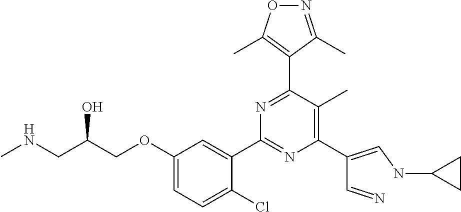 [SMILES: CNC[C@@H](O)COc1ccc(Cl)c(-c2nc(/C(C=N)=C/NC3CC3)c(C)c(-c3c(C)noc3C)n2)c1] | 0.0081 | 0.130 | 509.2 |
| 104 | 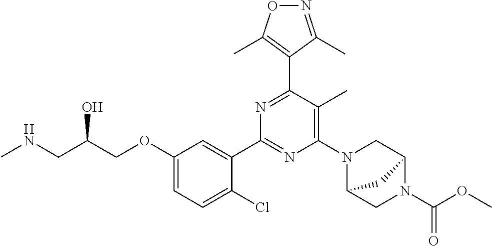 | 0.0108 | 0.368 | 557.1 |
| 105 | 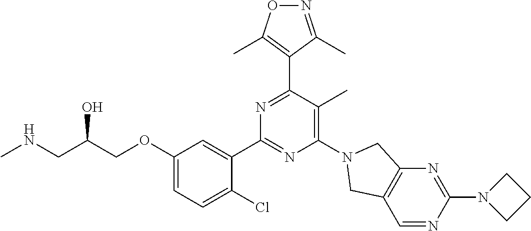 [SMILES: CNC[C@@H](O)COc1ccc(Cl)c(-c2nc(-c3c(C)noc3C)c(C)c(N3Cc4cnc(N5CCC5)nc4C3)n2)c1] | 0.0032 | 0.011 | 577 |
| 106 | 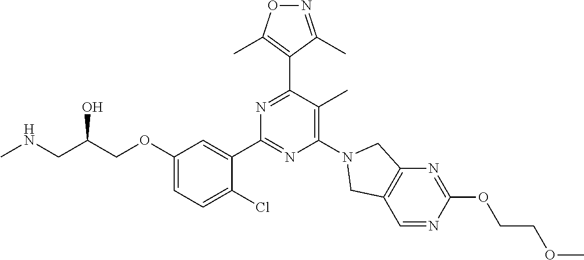 | 0.0055 | 0.025 | 596 |
| 107 | 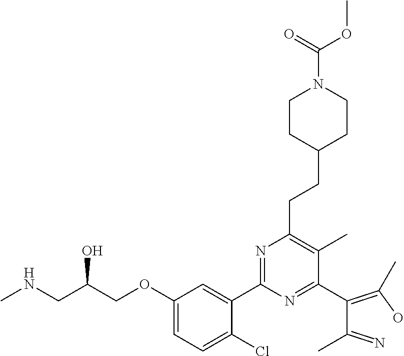 [SMILES: CNC[C@@H](O)COc1ccc(Cl)c(-c2nc(CCC3CCN(C(=O)OC)CC3)c(C)c(/C(C(C)=N)=C(\C)O)n2)c1] | 0.0112 | 0.176 | 572.2 |
| 108 | 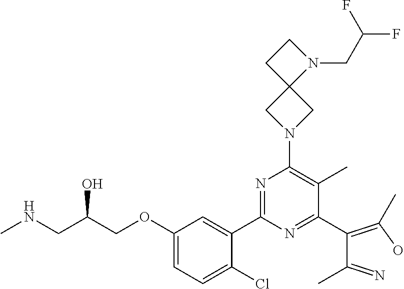 [SMILES: CNC[C@@H](O)COc1ccc(Cl)c(-c2nc(/C(C(C)=N)=C(\C)O)c(C)c(N3CC4(CCN4CC(F)F)C3)n2)c1] | 0.0183 | 0.164 | 563.2 |
| 109 | 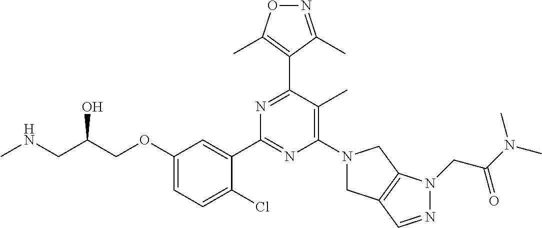 | 0.0064 | 2.841 | 595.2 |
| 110 | 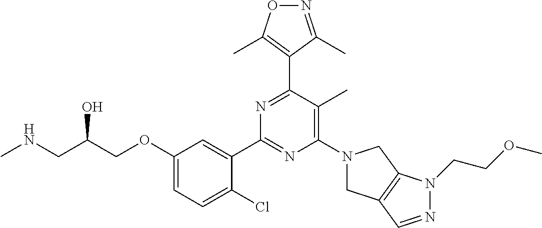 | 0.0048 | 0.052 | 568.2 |
| 111 | 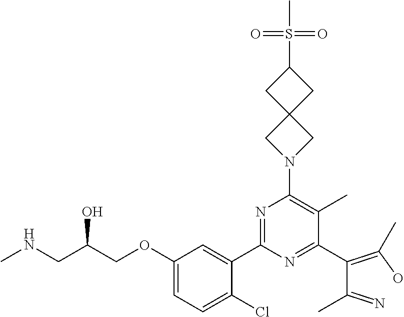 [SMILES: CNC[C@@H](O)COc1ccc(Cl)c(-c2nc(/C(C(C)=N)=C(\C)O)c(C)c(N3CC4(CC(S(C)(=O)=O)C4)C3)n2)c1] | 0.0112 | 0.822 | 576.2 |
| 112 | 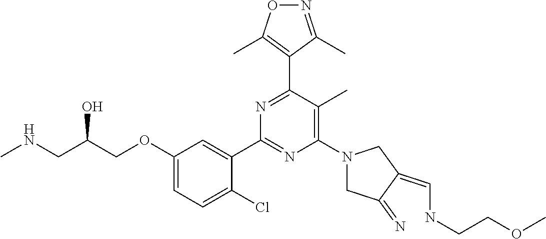 | 0.0072 | 0.066 | 568.2 |
| 113 | 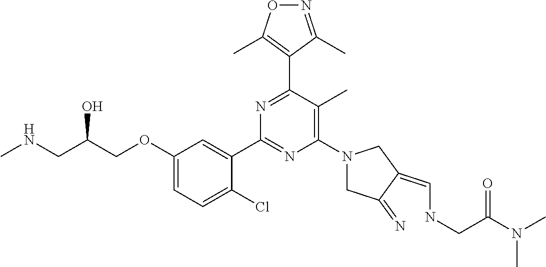 | 0.0088 | 1.149 | 595.2 |
| 114 | 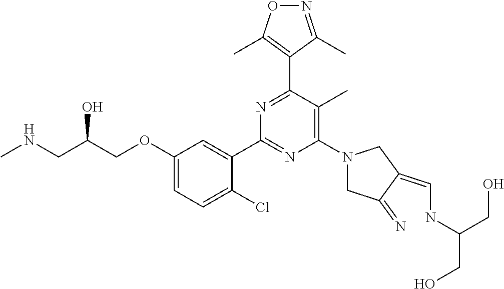 [SMILES: CNC[C@@H](O)COc1ccc(Cl)c(-c2nc(-c3c(C)noc3C)c(C)c(N3CC(=N)/C(=C\NC(CO)CO)C3)n2)c1] | 0.0078 | 5.847 | 584.2 |
| 115 | 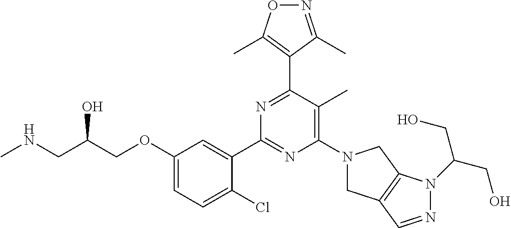 [SMILES: CNC[C@@H](O)COc1ccc(Cl)c(-c2nc(-c3c(C)noc3C)c(C)c(N3Cc4cnn(C(CO)CO)c4C3)n2)c1] | 0.0099 | 9.129 | 584.2 |
| 116 | 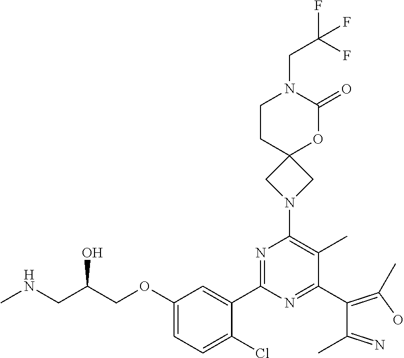 [SMILES: CNC[C@@H](O)COc1ccc(Cl)c(-c2nc(/C(C(C)=N)=C(\C)O)c(C)c(N3CC4(CCN(CC(F)(F)F)C(=O)O4)C3)n2)c1] | 0.0138 | 0.339 | 625 |
| 117 | 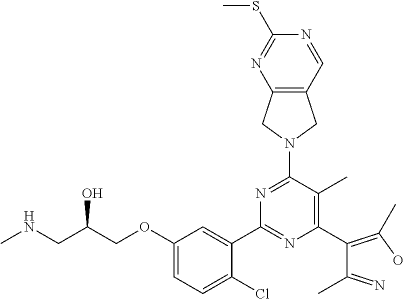 [SMILES: CNC[C@@H](O)COc1ccc(Cl)c(-c2nc(/C(C(C)=N)=C(\C)O)c(C)c(N3Cc4cnc(SC)nc4C3)n2)c1] | 0.0039 | 0.031 | 568.2 |
| 118 | 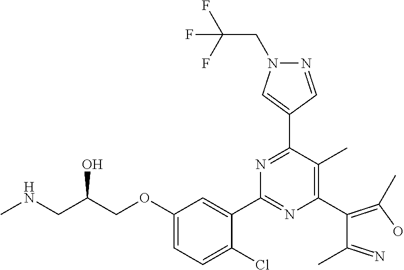 [SMILES: CNC[C@@H](O)COc1ccc(Cl)c(-c2nc(/C(C(C)=N)=C(\C)O)c(C)c(-c3cnn(CC(F)(F)F)c3)n2)c1] | 0.0090 | 0.428 | 551.2 |
| 119 | 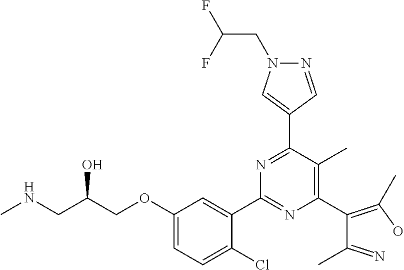 [SMILES: CNC[C@@H](O)COc1ccc(Cl)c(-c2nc(/C(C(C)=N)=C(\C)O)c(C)c(-c3cnn(CC(F)F)c3)n2)c1] | 0.0076 | 0.340 | 533.2 |
| 120 | 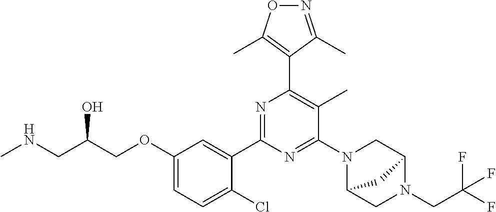 [SMILES: CNC[C@@H](O)COc1ccc(Cl)c(-c2nc(-c3c(C)noc3C)c(C)c(N3C[C@@H]4C[C@H]3CN4CC(F)(F)F)n2)c1] | 0.0088 | 0.389 | 581.1 |
| 121 | 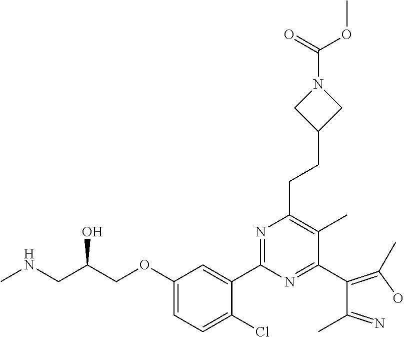 [SMILES: CNC[C@@H](O)COc1ccc(Cl)c(-c2nc(CCC3CN(C(=O)OC)C3)c(C)c(/C(C(C)=N)=C(\C)O)n2)c1] | 0.0134 | 0.257 | 544 |
| 122 | 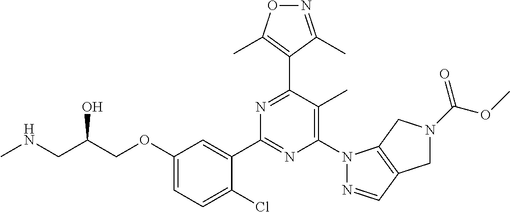 | 0.0137 | 1.810 | 568.1 |
| 123 | 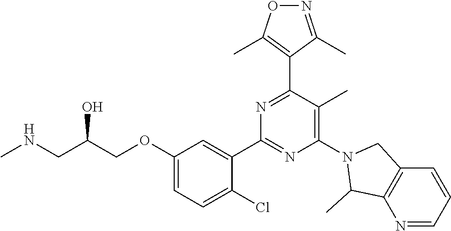 [SMILES: CNC[C@@H](O)COc1ccc(Cl)c(-c2nc(-c3c(C)noc3C)c(C)c(N3Cc4cccnc4C3C)n2)c1] | 0.0034 | 0.163 | 535 |
| 124 | 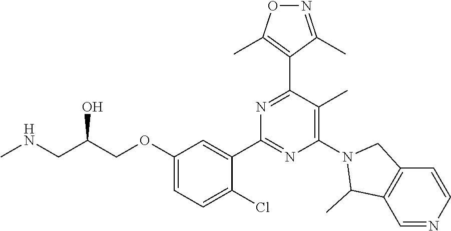 [SMILES: CNC[C@@H](O)COc1ccc(Cl)c(-c2nc(-c3c(C)noc3C)c(C)c(N3Cc4ccncc4C3C)n2)c1] | 0.0052 | 0.175 | 535 |
| 125 | 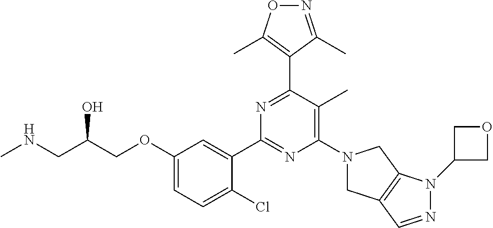 [SMILES: CNC[C@@H](O)COc1ccc(Cl)c(-c2nc(-c3c(C)noc3C)c(C)c(N3Cc4cnn(C5COC5)c4C3)n2)c1] | 0.0065 | 0.150 | 566.2 |
| 126 | 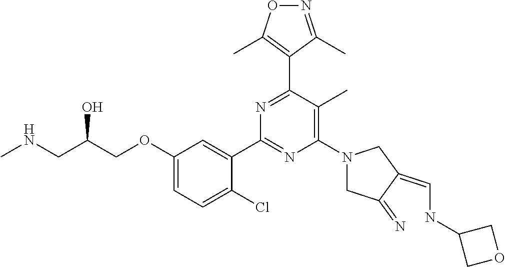 [SMILES: CNC[C@@H](O)COc1ccc(Cl)c(-c2nc(-c3c(C)noc3C)c(C)c(N3CC(=N)/C(=C\NC4COC4)C3)n2)c1] | 0.0062 | 0.157 | 566.2 |
| 127 | 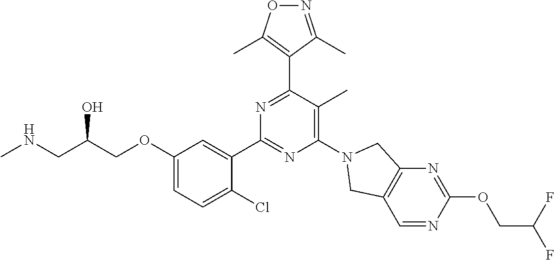 [SMILES: CNC[C@@H](O)COc1ccc(Cl)c(-c2nc(-c3c(C)noc3C)c(C)c(N3Cc4cnc(OCC(F)F)nc4C3)n2)c1] | 0.0078 | 0.032 | 602 |
| 128 | 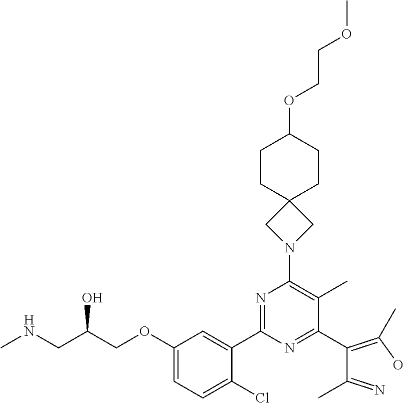 [SMILES: CNC[C@@H](O)COc1ccc(Cl)c(-c2nc(/C(C(C)=N)=C(\C)O)c(C)c(N3CC4(CCC(OCCOC)CC4)C3)n2)c1] | 0.0163 | 0.099 | 600 |
| 129 | 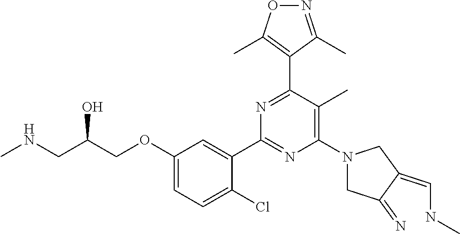 | 0.0077 | 0.046 | 524.2 |
| 130 | 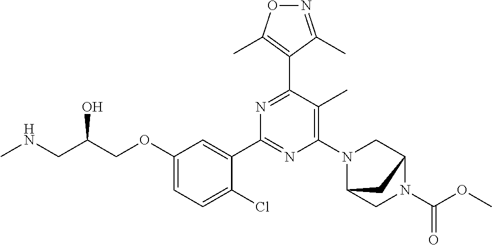 | 0.0059 | 0.960 | 557.1 |
| 131 | 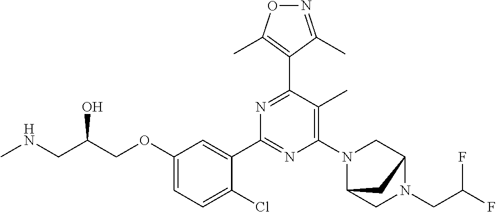 [SMILES: CNC[C@@H](O)COc1ccc(Cl)c(-c2nc(-c3c(C)noc3C)c(C)c(N3C[C@H]4C[C@@H]3CN4CC(F)F)n2)c1] | 0.0090 | 0.612 | 563 |
| 132 | 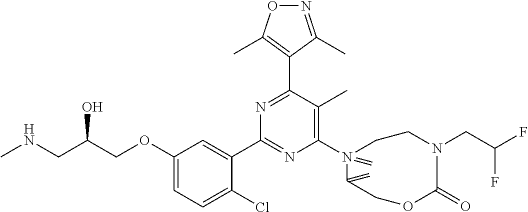 | 0.0066 | 0.637 | 607 |
| 133 | 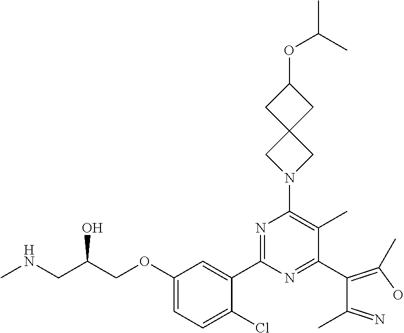 [SMILES: CNC[C@@H](O)COc1ccc(Cl)c(-c2nc(/C(C(C)=N)=C(\C)O)c(C)c(N3CC4(CC(OC(C)C)C4)C3)n2)c1] | 0.0099 | 0.042 | 556 |
| 134 | 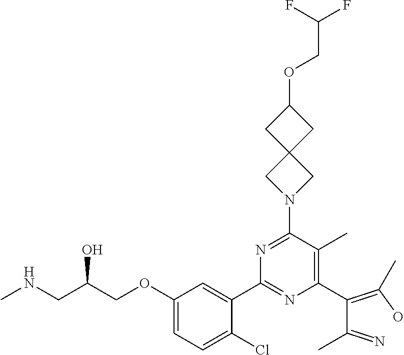 [SMILES: CNC[C@@H](O)COc1ccc(Cl)c(-c2nc(/C(C(C)=N)=C(\C)O)c(C)c(N3CC4(CC(OCC(F)F)C4)C3)n2)c1] | 0.0085 | 0.045 | 578 |
| 135 | 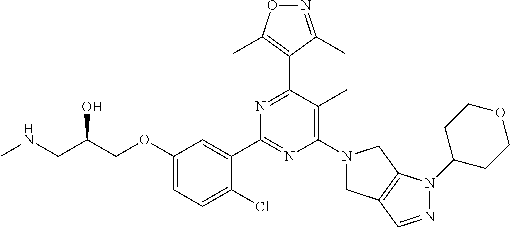 [SMILES: CNC[C@@H](O)COc1ccc(Cl)c(-c2nc(-c3c(C)noc3C)c(C)c(N3Cc4cnn(C5CCOCC5)c4C3)n2)c1] | 0.0046 | 0.093 | 594.2 |
| 136 | 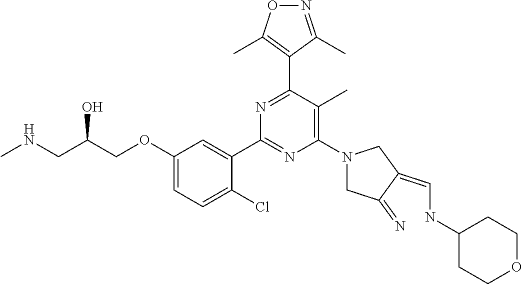 [SMILES: CNC[C@@H](O)COc1ccc(Cl)c(-c2nc(-c3c(C)noc3C)c(C)c(N3CC(=N)/C(=C\NC4CCOCC4)C3)n2)c1] | 0.0051 | 0.172 | 594.2 |
| 137 | 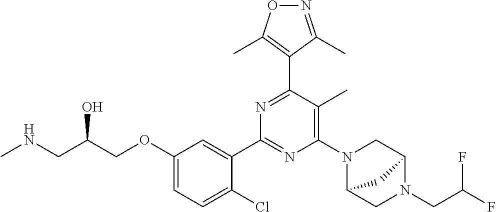 [SMILES: CNC[C@@H](O)COc1ccc(Cl)c(-c2nc(-c3c(C)noc3C)c(C)c(N3C[C@@H]4C[C@H]3CN4CC(F)F)n2)c1] | 0.0080 | 0.311 | 563.1 |
| 138 | 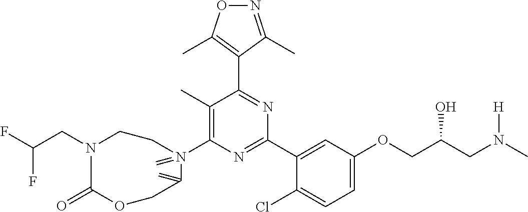 | 0.0046 | 0.581 | 607 |
| 139 | 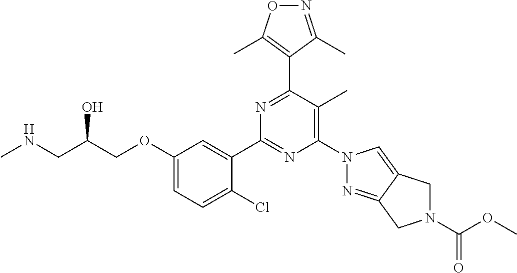 | 0.0052 | 0.131 | 568 |
| 140 | 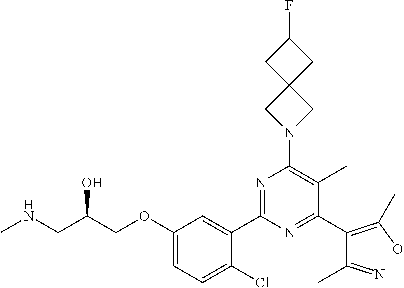 [SMILES: CNC[C@@H](O)COc1ccc(Cl)c(-c2nc(/C(C(C)=N)=C(\C)O)c(C)c(N3CC4(CC(F)C4)C3)n2)c1] | 0.0042 | 0.241 | 516 |
| 141 | 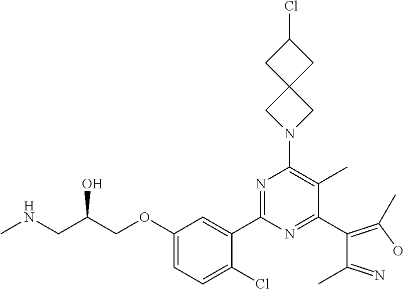 [SMILES: CNC[C@@H](O)COc1ccc(Cl)c(-c2nc(/C(C(C)=N)=C(\C)O)c(C)c(N3CC4(CC(Cl)C4)C3)n2)c1] | 0.0037 | 0.173 | 532 |
| 142 | 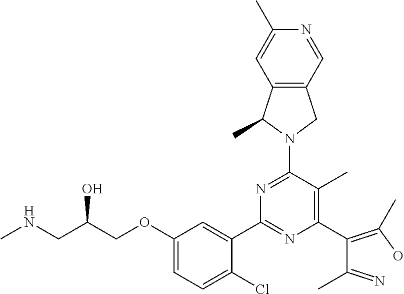 [SMILES: CNC[C@@H](O)COc1ccc(Cl)c(-c2nc(/C(C(C)=N)=C(\C)O)c(C)c(N3Cc4cnc(C)cc4[C@@H]3C)n2)c1] | 0.0036 | 0.065 | 549.2 |
| 143 | 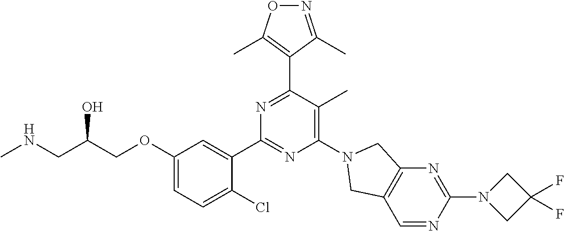 [SMILES: CNC[C@@H](O)COc1ccc(Cl)c(-c2nc(-c3c(C)noc3C)c(C)c(N3Cc4cnc(N5CC(F)(F)C5)nc4C3)n2)c1] | 0.0028 | 0.015 | 613 |
| 144 | 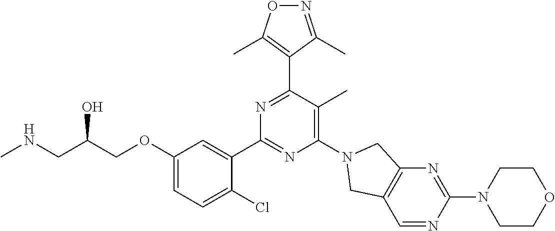 [SMILES: CNC[C@@H](O)COc1ccc(Cl)c(-c2nc(-c3c(C)noc3C)c(C)c(N3Cc4cnc(N5CCOCC5)nc4C3)n2)c1] | 0.0033 | 0.022 | 607.2 |
| 145 | 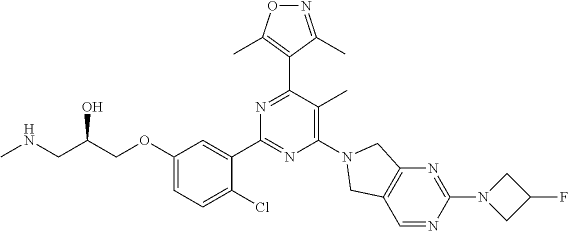 [SMILES: CNC[C@@H](O)COc1ccc(Cl)c(-c2nc(-c3c(C)noc3C)c(C)c(N3Cc4cnc(N5CC(F)C5)nc4C3)n2)c1] | 0.0020 | 0.015 | 595 |
| 146 | 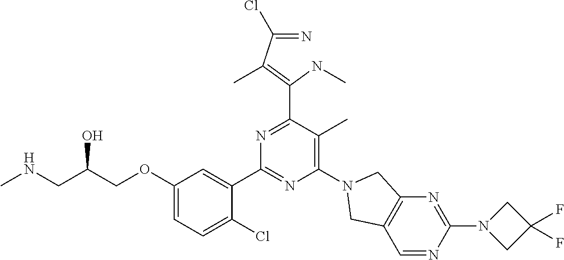 [SMILES: CNC[C@@H](O)COc1ccc(Cl)c(-c2nc(/C(NC)=C(\C)C(=N)Cl)c(C)c(N3Cc4cnc(N5CC(F)(F)C5)nc4C3)n2)c1] | 0.0027 | 0.026 | 646.2 |
| 147 | 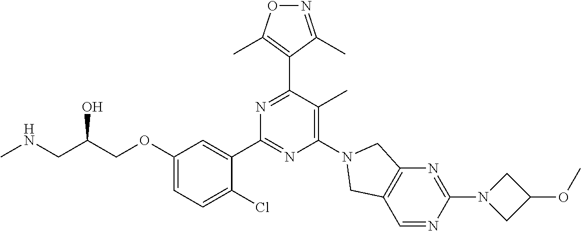 | 0.0028 | 0.016 | 607.1 |
| 148 | 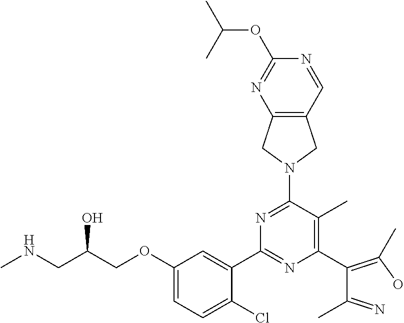 [SMILES: CNC[C@@H](O)COc1ccc(Cl)c(-c2nc(/C(C(C)=N)=C(\C)O)c(C)c(N3Cc4cnc(OC(C)C)nc4C3)n2)c1] | 0.0037 | 0.039 | 580 |
| 149 | 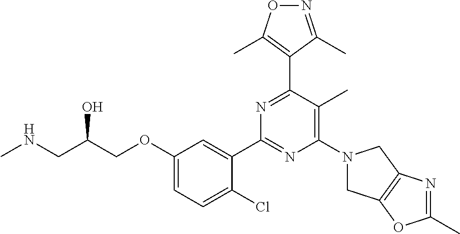 | 0.0031 | 0.052 | 525.2 |
| 150 | 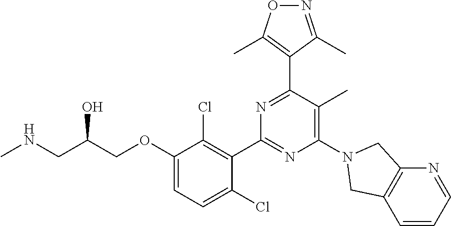 [SMILES: CNC[C@@H](O)COc1ccc(Cl)c(-c2nc(-c3c(C)noc3C)c(C)c(N3Cc4cccnc4C3)n2)c1Cl] | 0.0162 | 2.020 | 555.2 |
| 151 | 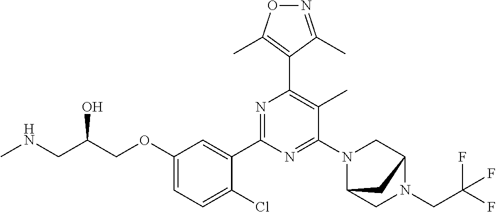 [SMILES: CNC[C@@H](O)COc1ccc(Cl)c(-c2nc(-c3c(C)noc3C)c(C)c(N3C[C@H]4C[C@@H]3CN4CC(F)(F)F)n2)c1] | 0.0121 | 0.668 | 581.1 |
| 152 | 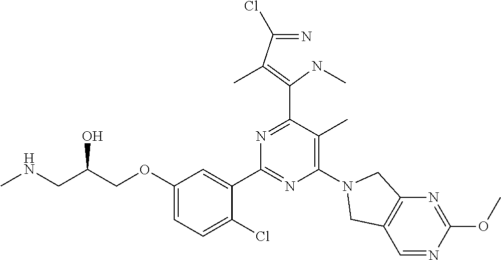 | 0.0019 | 0.018 | 585 |
| 153 | 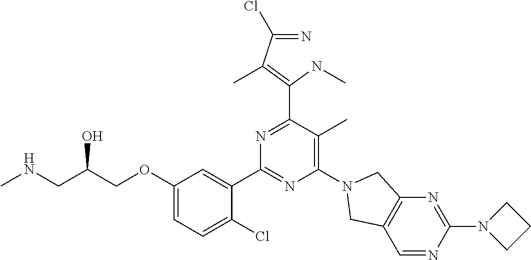 [SMILES: CNC[C@@H](O)COc1ccc(Cl)c(-c2nc(/C(NC)=C(\C)C(=N)Cl)c(C)c(N3Cc4cnc(N5CCC5)nc4C3)n2)c1] | 0.0019 | 0.015 | 610.1 |
| 154 | 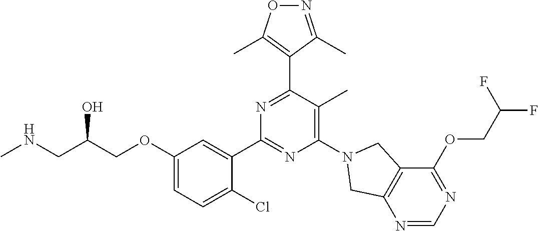 [SMILES: CNC[C@@H](O)COc1ccc(Cl)c(-c2nc(-c3c(C)noc3C)c(C)c(N3Cc4ncnc(OCC(F)F)c4C3)n2)c1] | 0.0031 | 0.057 | 602.1 |
| 155 | 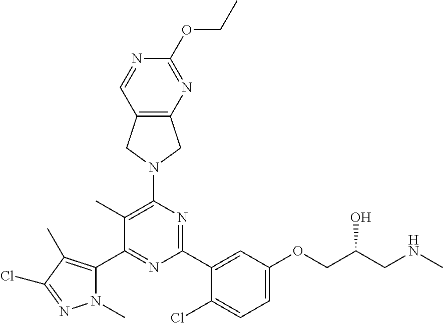 [SMILES: CCOc1ncc2c(n1)CN(c1nc(-c3cc(OC[C@H](O)CNC)ccc3Cl)nc(-c3c(C)c(Cl)nn3C)c1C)C2] | 0.0024 | 0.018 | 599 |
| 156 | 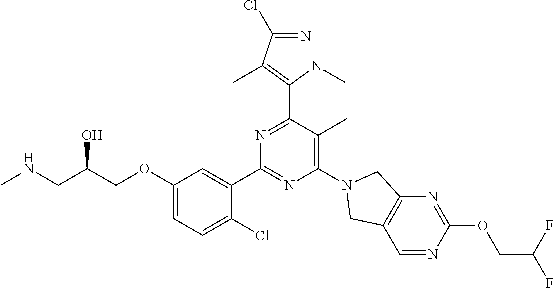 [SMILES: CNC[C@@H](O)COc1ccc(Cl)c(-c2nc(/C(NC)=C(\C)C(=N)Cl)c(C)c(N3Cc4cnc(OCC(F)F)nc4C3)n2)c1] | 0.0035 | 0.031 | 635 |
| 157 | 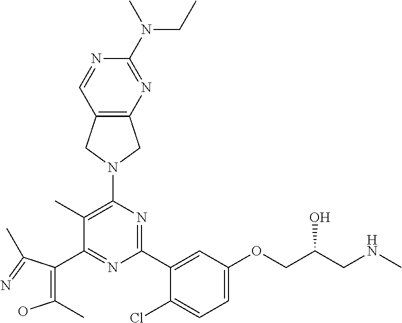 [SMILES: CCN(C)c1ncc2c(n1)CN(c1nc(-c3cc(OC[C@H](O)CNC)ccc3Cl)nc(/C(C(C)=N)=C(\C)O)c1C)C2] | 0.0024 | 0.026 | 579.1 |
| 158 | 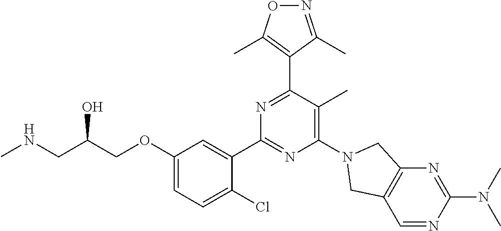 | 0.0029 | 0.018 | 565.1 |
| 159 | 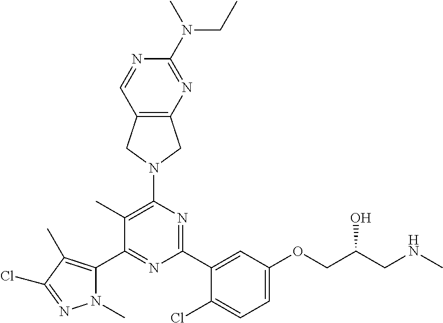 [SMILES: CCN(C)c1ncc2c(n1)CN(c1nc(-c3cc(OC[C@H](O)CNC)ccc3Cl)nc(-c3c(C)c(Cl)nn3C)c1C)C2] | 0.0020 | 0.039 | 612 |
| 160 | 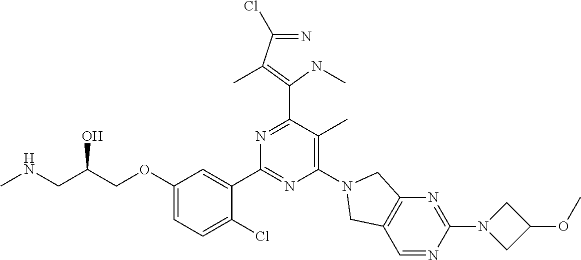 | 0.0030 | 0.026 | 641.2 |
| 161 | 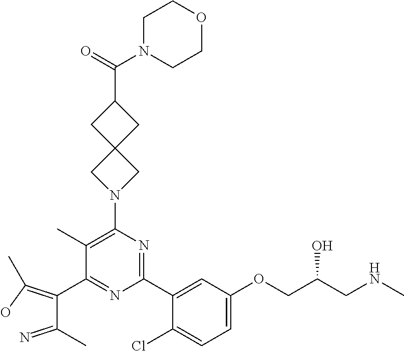 [SMILES: CNC[C@@H](O)COc1ccc(Cl)c(-c2nc(/C(C(C)=N)=C(\C)O)c(C)c(N3CC4(CC(C(=O)N5CCOCC5)C4)C3)n2)c1] | 0.0074 | 0.239 | 611.2 |
| 162 | 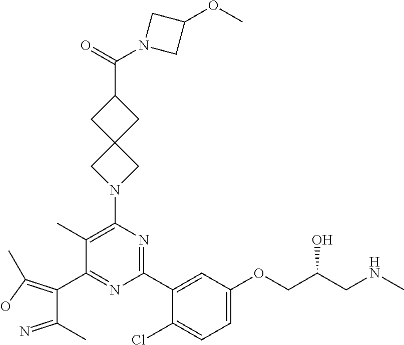 [SMILES: CNC[C@@H](O)COc1ccc(Cl)c(-c2nc(/C(C(C)=N)=C(\C)O)c(C)c(N3CC4(CC(C(=O)N5CC(OC)C5)C4)C3)n2)c1] | 0.0069 | 0.550 | 611.2 |
| 163 | 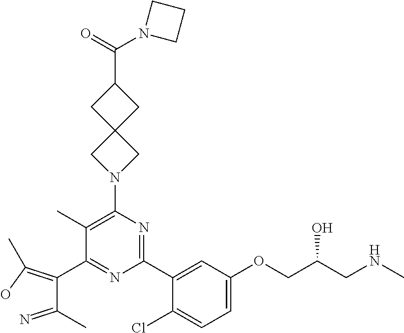 [SMILES: CNC[C@@H](O)COc1ccc(Cl)c(-c2nc(/C(C(C)=N)=C(\C)O)c(C)c(N3CC4(CC(C(=O)N5CCC5)C4)C3)n2)c1] | 0.0062 | 0.292 | 581.2 |
| 164 | 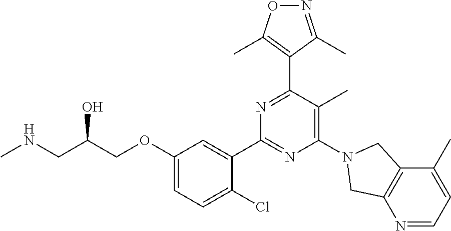 | 0.0022 | 0.055 | 535 |
| 165 | 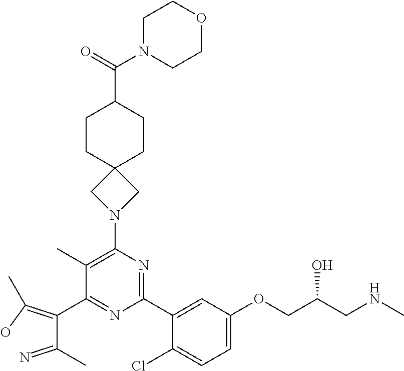 [SMILES: CNC[C@@H](O)COc1ccc(Cl)c(-c2nc(/C(C(C)=N)=C(\C)O)c(C)c(N3CC4(CCC(C(=O)N5CCOCC5)CC4)C3)n2)c1] | 0.0058 | 0.322 | 639 |
| 166 | 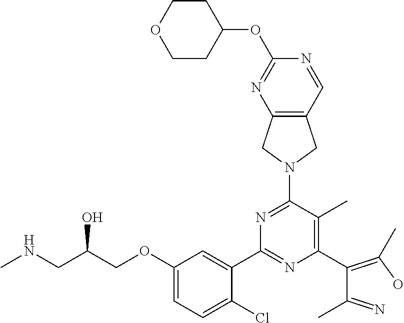 [SMILES: CNC[C@@H](O)COc1ccc(Cl)c(-c2nc(/C(C(C)=N)=C(\C)O)c(C)c(N3Cc4cnc(OC5CCOCC5)nc4C3)n2)c1] | 0.0051 | 0.049 | 622.2 |
| 167 | 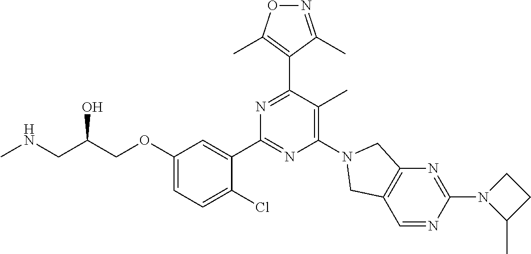 [SMILES: CNC[C@@H](O)COc1ccc(Cl)c(-c2nc(-c3c(C)noc3C)c(C)c(N3Cc4cnc(N5CCC5C)nc4C3)n2)c1] | 0.0019 | 0.019 | 591.1 |
| 168 | 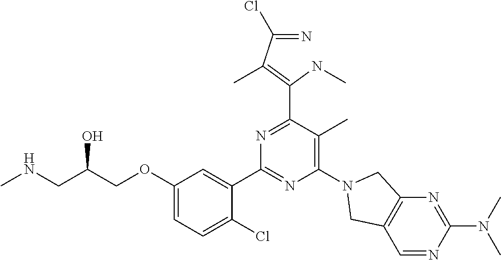 | 0.0025 | 0.026 | 598 |
| 169 | 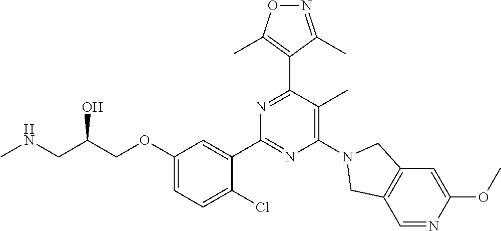 | 0.0021 | 0.034 | 551.1 |
| 170 | 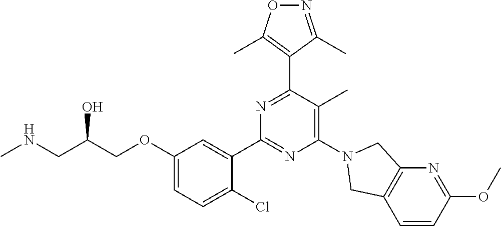 | 0.0019 | 0.038 | 551 |
| 171 | 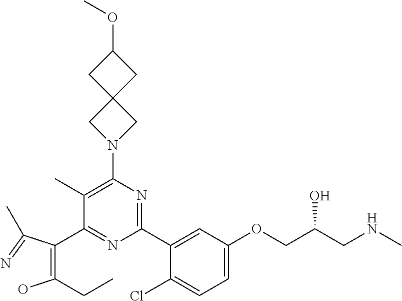 [SMILES: CC/C(O)=C(/C(C)=N)c1nc(-c2cc(OC[C@H](O)CNC)ccc2Cl)nc(N2CC3(CC(OC)C3)C2)c1C] | 0.0058 | 0.174 | 542.1 |
| 172 | 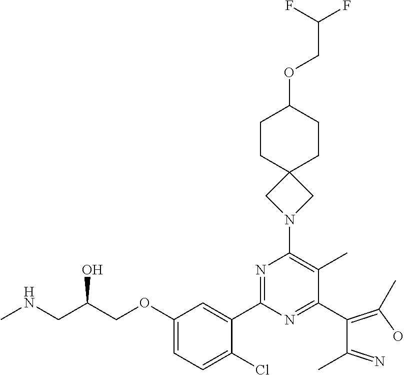 [SMILES: CNC[C@@H](O)COc1ccc(Cl)c(-c2nc(/C(C(C)=N)=C(\C)O)c(C)c(N3CC4(CCC(OCC(F)F)CC4)C3)n2)c1] | 0.0047 | 0.057 | 606 |
| 173 | 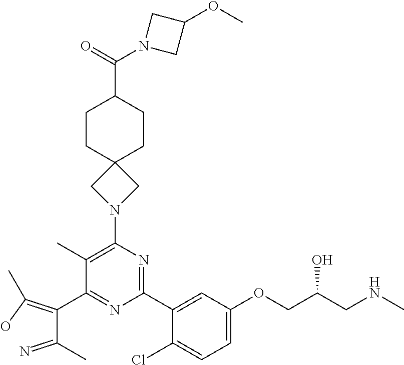 [SMILES: CNC[C@@H](O)COc1ccc(Cl)c(-c2nc(/C(C(C)=N)=C(\C)O)c(C)c(N3CC4(CCC(C(=O)N5CC(OC)C5)CC4)C3)n2)c1] | 0.0050 | 0.408 | 639 |
| 174 | 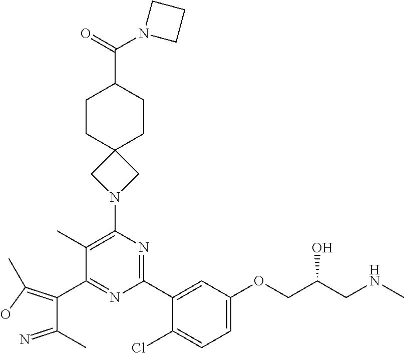 [SMILES: CNC[C@@H](O)COc1ccc(Cl)c(-c2nc(/C(C(C)=N)=C(\C)O)c(C)c(N3CC4(CCC(C(=O)N5CCC5)CC4)C3)n2)c1] | 0.0039 | 0.271 | 609 |
| 175 | 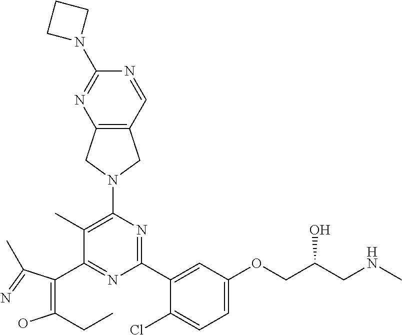 [SMILES: CC/C(O)=C(/C(C)=N)c1nc(-c2cc(OC[C@H](O)CNC)ccc2Cl)nc(N2Cc3cnc(N4CCC4)nc3C2)c1C] | 0.0017 | 0.013 | 591.2 |
| 176 | 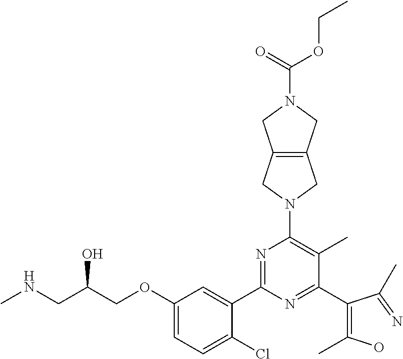 [SMILES: CCOC(=O)N1CC2=C(C1)CN(c1nc(-c3cc(OC[C@H](O)CNC)ccc3Cl)nc(/C(C(C)=N)=C(\C)O)c1C)C2] | 0.0027 | 0.026 | 583 |
| 177 | 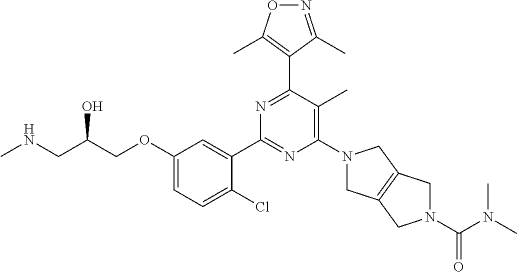 | 0.0036 | 0.087 | 582 |
| 178 | 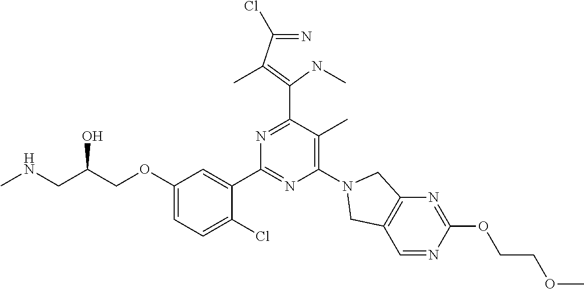 | 0.0014 | 0.024 | 629.2 |
| 179 | 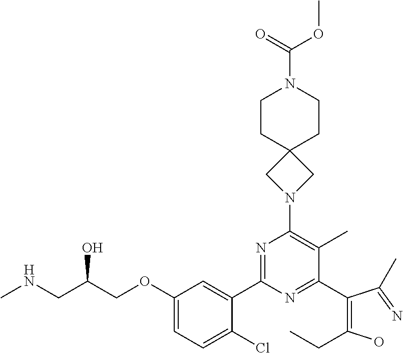 [SMILES: CC/C(O)=C(/C(C)=N)c1nc(-c2cc(OC[C@H](O)CNC)ccc2Cl)nc(N2CC3(CCN(C(=O)OC)CC3)C2)c1C] | 0.0051 | 0.119 | 599.1 |
| 180 | 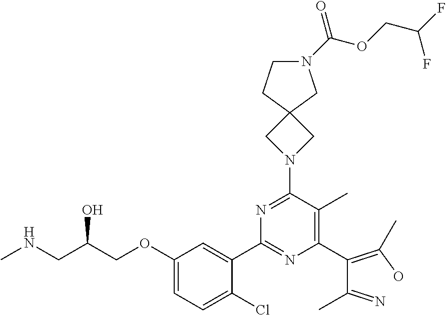 [SMILES: CNC[C@@H](O)COc1ccc(Cl)c(-c2nc(/C(C(C)=N)=C(\C)O)c(C)c(N3CC4(CCN(C(=O)OCC(F)F)C4)C3)n2)c1] | 0.0041 | 0.121 | 621.2 |
| 181 | 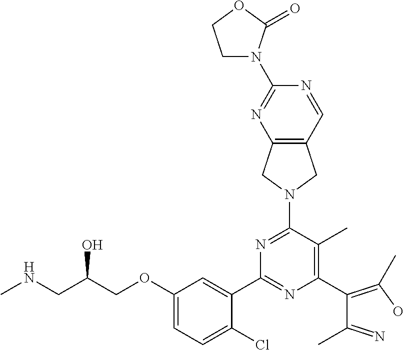 [SMILES: CNC[C@@H](O)COc1ccc(Cl)c(-c2nc(/C(C(C)=N)=C(\C)O)c(C)c(N3Cc4cnc(N5CCOC5=O)nc4C3)n2)c1] | 0.0018 | 0.459 | 607.2 |
| 182 | 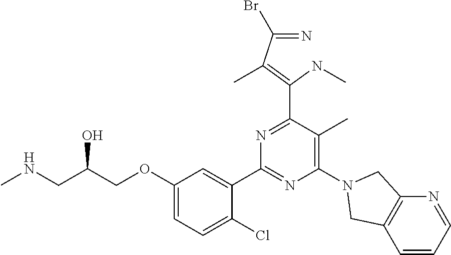 [SMILES: CNC[C@@H](O)COc1ccc(Cl)c(-c2nc(/C(NC)=C(\C)C(=N)Br)c(C)c(N3Cc4cccnc4C3)n2)c1] | 0.0008 | 0.021 | 600.1 |
| 183 | 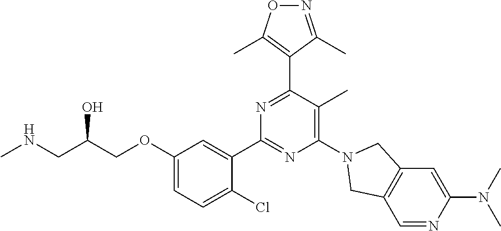 | 0.0018 | 0.031 | 564.2 |
| 184 | 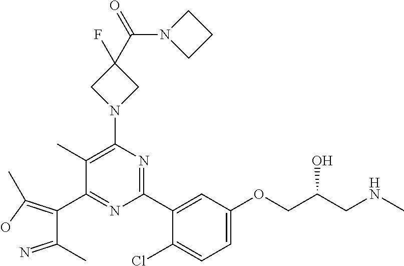 [SMILES: CNC[C@@H](O)COc1ccc(Cl)c(-c2nc(/C(C(C)=N)=C(\C)O)c(C)c(N3CC(F)(C(=O)N4CCC4)C3)n2)c1] | 0.0061 | 0.488 | 559.2 |
| 185 | 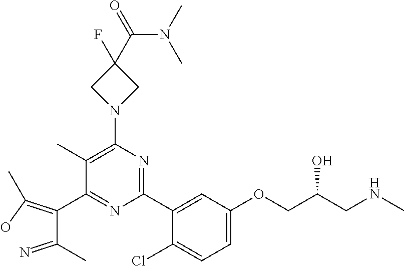 [SMILES: CNC[C@@H](O)COc1ccc(Cl)c(-c2nc(/C(C(C)=N)=C(\C)O)c(C)c(N3CC(F)(C(=O)N(C)C)C3)n2)c1] | 0.0051 | 0.346 | 547 |
| 186 | 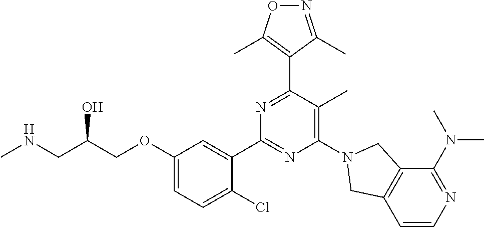 | 0.0018 | 0.049 | 564.2 |
| 187 | 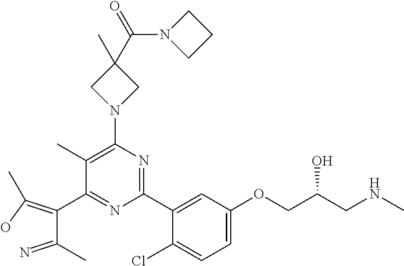 [SMILES: CNC[C@@H](O)COc1ccc(Cl)c(-c2nc(/C(C(C)=N)=C(\C)O)c(C)c(N3CC(C)(C(=O)N4CCC4)C3)n2)c1] | 0.0058 | 0.337 | 555 |
| 188 | 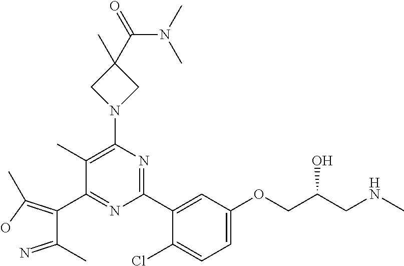 [SMILES: CNC[C@@H](O)COc1ccc(Cl)c(-c2nc(/C(C(C)=N)=C(\C)O)c(C)c(N3CC(C)(C(=O)N(C)C)C3)n2)c1] | 0.0052 | 0.150 | 543 |
| 189 | 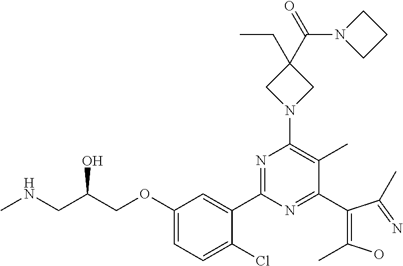 [SMILES: CCC1(C(=O)N2CCC2)CN(c2nc(-c3cc(OC[C@H](O)CNC)ccc3Cl)nc(/C(C(C)=N)=C(\C)O)c2C)C1] | 0.0057 | 0.246 | 569.3 |
| 190 | 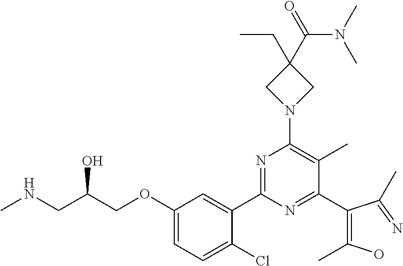 [SMILES: CCC1(C(=O)N(C)C)CN(c2nc(-c3cc(OC[C@H](O)CNC)ccc3Cl)nc(/C(C(C)=N)=C(\C)O)c2C)C1] | 0.0062 | 0.138 | 557.3 |
| 191 | 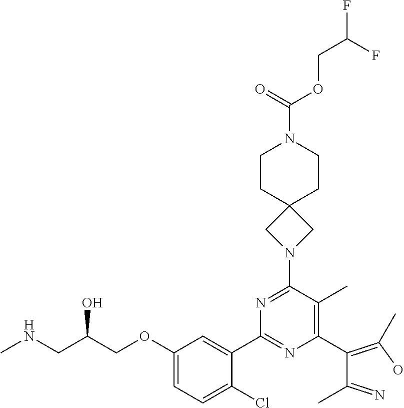 [SMILES: CNC[C@@H](O)COc1ccc(Cl)c(-c2nc(/C(C(C)=N)=C(\C)O)c(C)c(N3CC4(CCN(C(=O)OCC(F)F)CC4)C3)n2)c1] | 0.0105 | 0.174 | 635.2 |
| 192 | 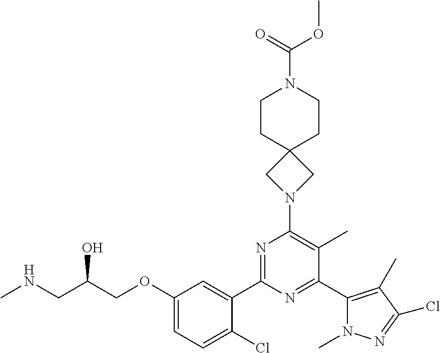 [SMILES: CNC[C@@H](O)COc1ccc(Cl)c(-c2nc(-c3c(C)c(Cl)nn3C)c(C)c(N3CC4(CCN(C(=O)OC)CC4)C3)n2)c1] | 0.0047 | 0.058 | 617.2 |
| 193 | 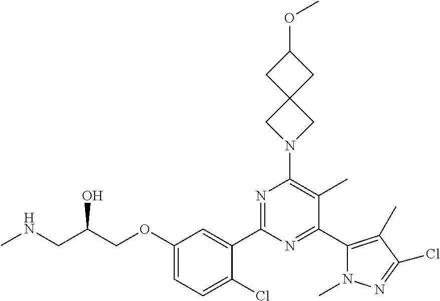 [SMILES: CNC[C@@H](O)COc1ccc(Cl)c(-c2nc(-c3c(C)c(Cl)nn3C)c(C)c(N3CC4(CC(OC)C4)C3)n2)c1] | 0.0041 | 0.052 | 561.2 |
| 194 | 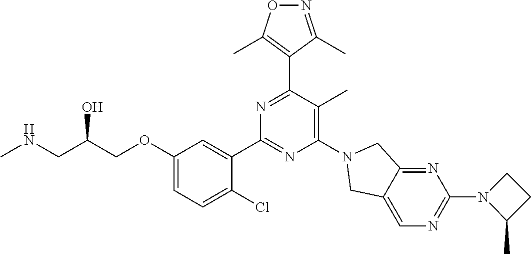 [SMILES: CNC[C@@H](O)COc1ccc(Cl)c(-c2nc(-c3c(C)noc3C)c(C)c(N3Cc4cnc(N5CC[C@H]5C)nc4C3)n2)c1] | 0.0018 | 0.013 | 591 |
| 195 | 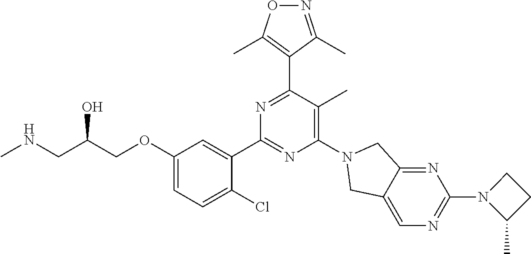 [SMILES: CNC[C@@H](O)COc1ccc(Cl)c(-c2nc(-c3c(C)noc3C)c(C)c(N3Cc4cnc(N5CC[C@@H]5C)nc4C3)n2)c1] | 0.0018 | 0.013 | 591 |
| 196 | 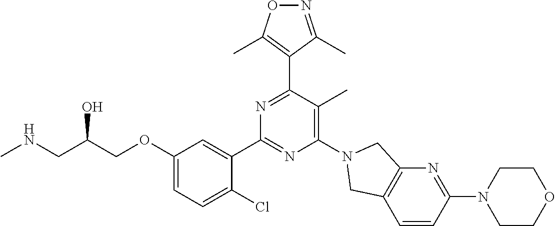 [SMILES: CNC[C@@H](O)COc1ccc(Cl)c(-c2nc(-c3c(C)noc3C)c(C)c(N3Cc4ccc(N5CCOCC5)nc4C3)n2)c1] | 0.0020 | 0.020 | 606.2 |
| 197 | 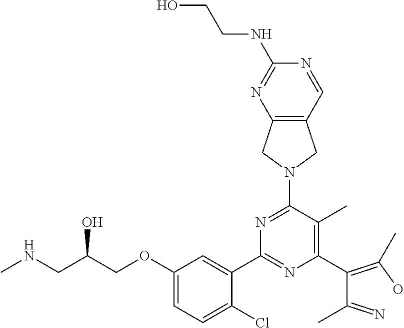 [SMILES: CNC[C@@H](O)COc1ccc(Cl)c(-c2nc(/C(C(C)=N)=C(\C)O)c(C)c(N3Cc4cnc(NCCO)nc4C3)n2)c1] | 0.0026 | 0.381 | 581.3 |
| 198 | 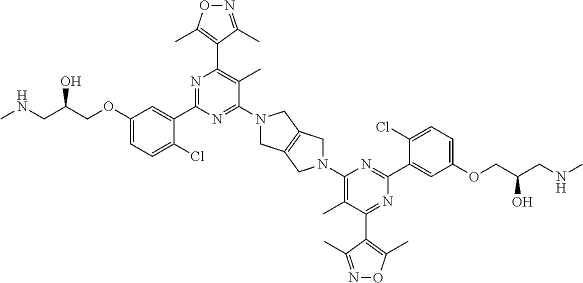 | 0.0004 | 0.171 | 911.3 |
| 199 | 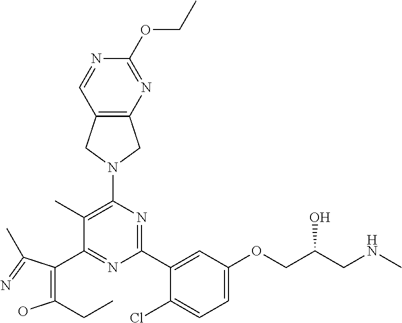 [SMILES: CCOc1ncc2c(n1)CN(c1nc(-c3cc(OC[C@H](O)CNC)ccc3Cl)nc(/C(C(C)=N)=C(/O)CC)c1C)C2] | 0.0057 | 0.084 | 580.2 |
| 200 | 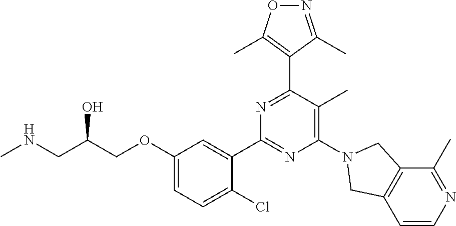 | 0.0021 | 0.047 | 536.1 |
| 201 | 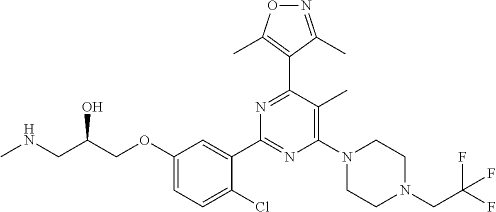 [SMILES: CNC[C@@H](O)COc1ccc(Cl)c(-c2nc(-c3c(C)noc3C)c(C)c(N3CCN(CC(F)(F)F)CC3)n2)c1] | 0.0048 | 0.295 | 569 |
| 202 | 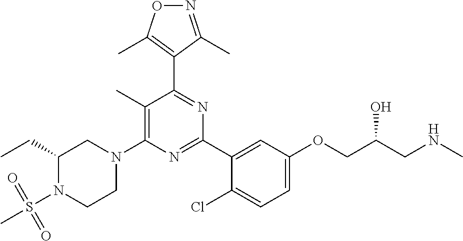 [SMILES: CC[C@@H]1CN(c2nc(-c3cc(OC[C@H](O)CNC)ccc3Cl)nc(-c3c(C)noc3C)c2C)CCN1S(C)(=O)=O] | 0.0033 | 0.233 | 593 |
| 203 | 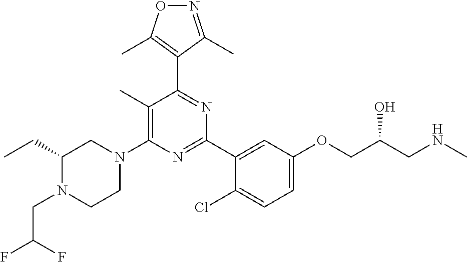 [SMILES: CC[C@@H]1CN(c2nc(-c3cc(OC[C@H](O)CNC)ccc3Cl)nc(-c3c(C)noc3C)c2C)CCN1CC(F)F] | 0.0046 | 0.200 | 579 |
| 204 | 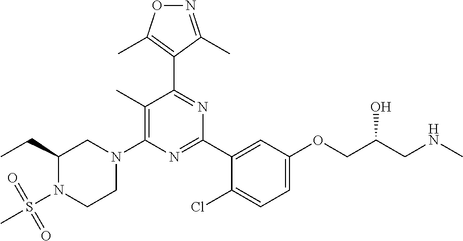 [SMILES: CC[C@H]1CN(c2nc(-c3cc(OC[C@H](O)CNC)ccc3Cl)nc(-c3c(C)noc3C)c2C)CCN1S(C)(=O)=O] | 0.0043 | 0.486 | 593 |
| 205 | 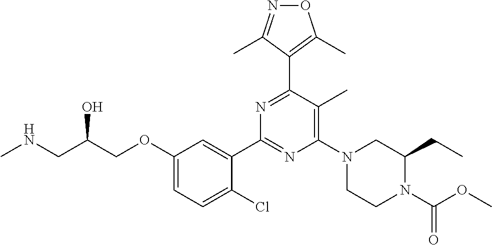 | 0.0037 | 0.202 | 573.1 |
| 206 | 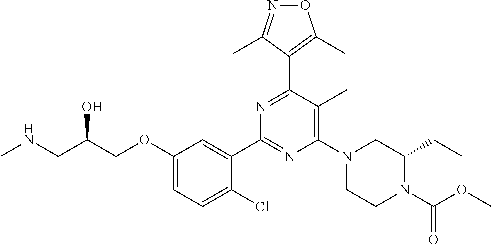 | 0.0054 | 0.696 | 573 |
| 207 | 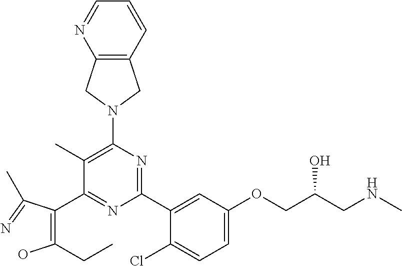 [SMILES: CC/C(O)=C(/C(C)=N)c1nc(-c2cc(OC[C@H](O)CNC)ccc2Cl)nc(N2Cc3cccnc3C2)c1C] | 0.0015 | 0.074 | 535.2 |
| 208 | 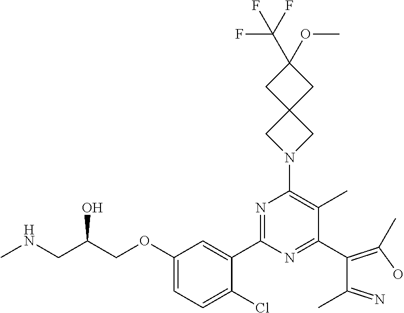 [SMILES: CNC[C@@H](O)COc1ccc(Cl)c(-c2nc(/C(C(C)=N)=C(\C)O)c(C)c(N3CC4(C3)CC(OC)(C(F)(F)F)C4)n2)c1] | 0.0052 | 0.142 | 596.3 |
| 209 | 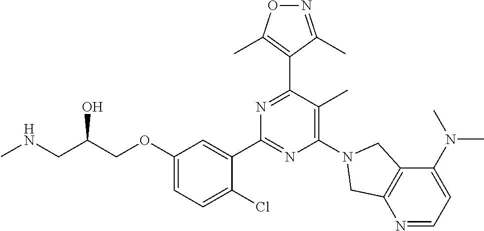 | 0.0006 | 0.129 | 564.2 |
| 210 | 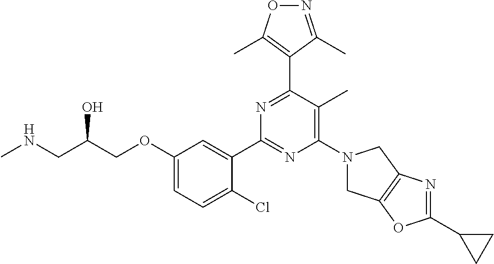 [SMILES: CNC[C@@H](O)COc1ccc(Cl)c(-c2nc(-c3c(C)noc3C)c(C)c(N3Cc4nc(C5CC5)oc4C3)n2)c1] | 0.0021 | 0.120 | 551 |
| 211 | 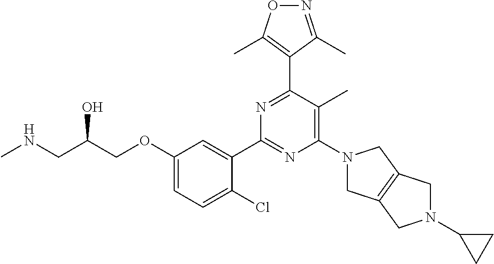 [SMILES: CNC[C@@H](O)COc1ccc(Cl)c(-c2nc(-c3c(C)noc3C)c(C)c(N3CC4=C(C3)CN(C3CC3)C4)n2)c1] | 0.0017 | 0.075 | 551.2 |
| 212 | 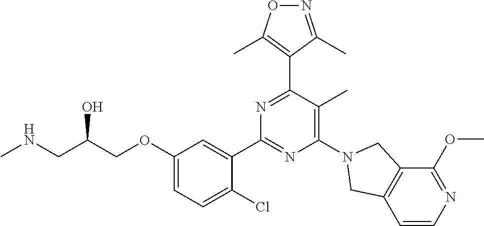 | 0.0021 | 0.040 | 551.1 |
| 213 | 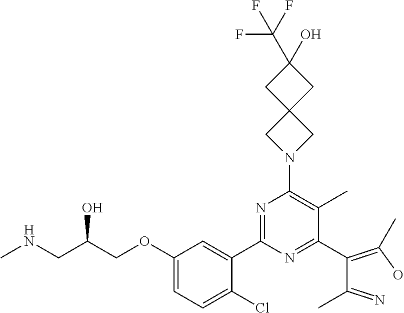 [SMILES: CNC[C@@H](O)COc1ccc(Cl)c(-c2nc(/C(C(C)=N)=C(\C)O)c(C)c(N3CC4(C3)CC(O)(C(F)(F)F)C4)n2)c1] | 0.0054 | 0.330 | 582.2 |
| 214 | 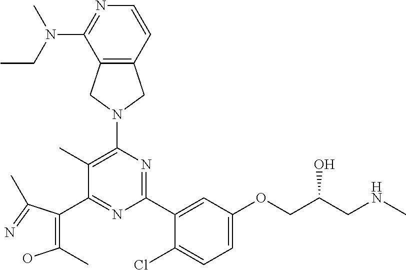 [SMILES: CCN(C)c1nccc2c1CN(c1nc(-c3cc(OC[C@H](O)CNC)ccc3Cl)nc(/C(C(C)=N)=C(\C)O)c1C)C2] | 0.0011 | 0.046 | 578.2 |
| 215 | 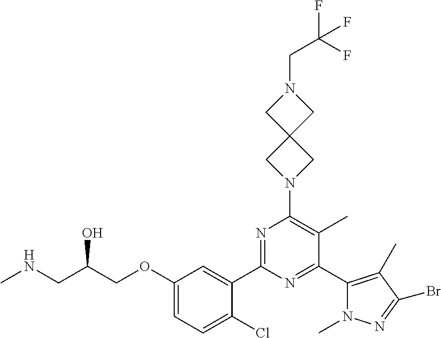 [SMILES: CNC[C@@H](O)COc1ccc(Cl)c(-c2nc(-c3c(C)c(Br)nn3C)c(C)c(N3CC4(CN(CC(F)(F)F)C4)C3)n2)c1] | 0.0044 | 0.023 | 660.1 |
| 216 | 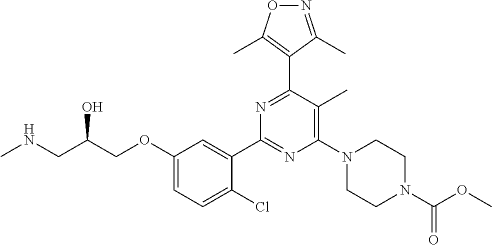 | 0.0054 | 0.415 | 545 |
| 217 | 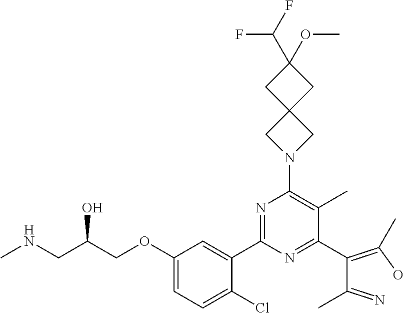 [SMILES: CNC[C@@H](O)COc1ccc(Cl)c(-c2nc(/C(C(C)=N)=C(\C)O)c(C)c(N3CC4(C3)CC(OC)(C(F)F)C4)n2)c1] | 0.0047 | 0.101 | 578.3 |
| 218 | 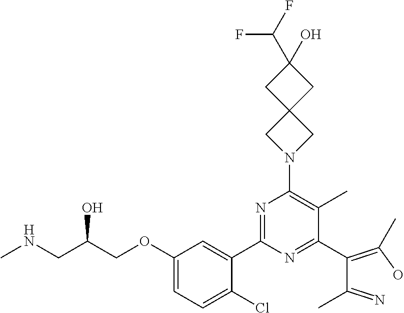 [SMILES: CNC[C@@H](O)COc1ccc(Cl)c(-c2nc(/C(C(C)=N)=C(\C)O)c(C)c(N3CC4(C3)CC(O)(C(F)F)C4)n2)c1] | 0.0060 | 0.419 | 564.02 |
| 219 | 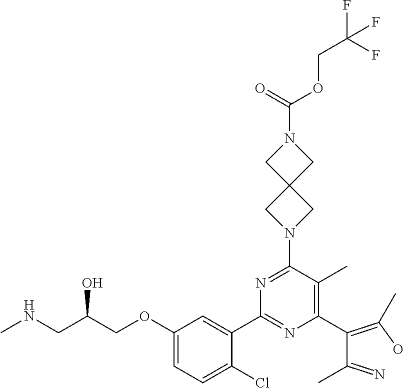 [SMILES: CNC[C@@H](O)COc1ccc(Cl)c(-c2nc(/C(C(C)=N)=C(\C)O)c(C)c(N3CC4(CN(C(=O)OCC(F)(F)F)C4)C3)n2)c1] | 0.0055 | 0.129 | 625 |
| 220 | 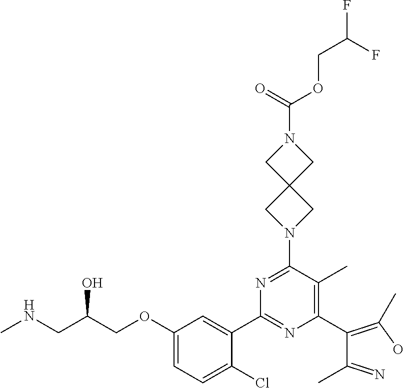 [SMILES: CNC[C@@H](O)COc1ccc(Cl)c(-c2nc(/C(C(C)=N)=C(\C)O)c(C)c(N3CC4(CN(C(=O)OCC(F)F)C4)C3)n2)c1] | 0.0042 | 0.221 | 607.1 |
| 221 | 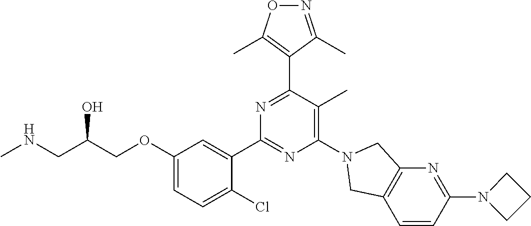 [SMILES: CNC[C@@H](O)COc1ccc(Cl)c(-c2nc(-c3c(C)noc3C)c(C)c(N3Cc4ccc(N5CCC5)nc4C3)n2)c1] | 0.0015 | 0.017 | 576 |
| 222 | 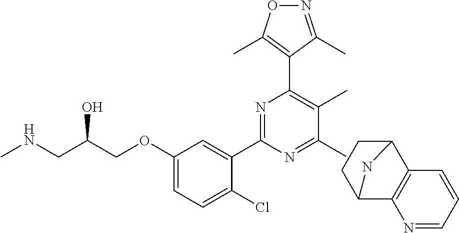 | 0.0060 | 1.396 | 547.2 |
| 223 | 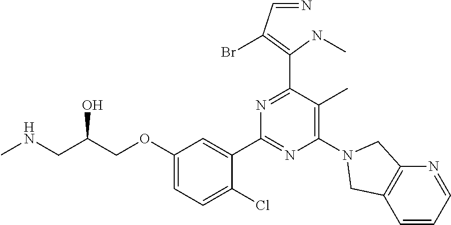 [SMILES: CNC[C@@H](O)COc1ccc(Cl)c(-c2nc(/C(NC)=C(\Br)C=N)c(C)c(N3Cc4cccnc4C3)n2)c1] | 0.0012 | 0.071 | 586 |
| 224 | 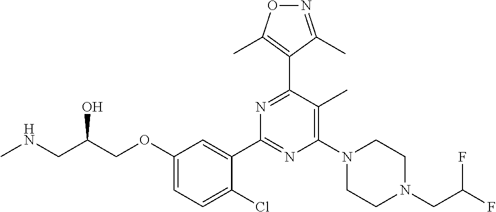 [SMILES: CNC[C@@H](O)COc1ccc(Cl)c(-c2nc(-c3c(C)noc3C)c(C)c(N3CCN(CC(F)F)CC3)n2)c1] | 0.0068 | 0.330 | 551.2 |
| 225 | 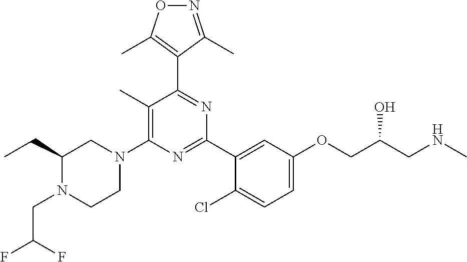 [SMILES: CC[C@H]1CN(c2nc(-c3cc(OC[C@H](O)CNC)ccc3Cl)nc(-c3c(C)noc3C)c2C)CCN1CC(F)F] | 0.0093 | 0.445 | 579.1 |
| 226 | 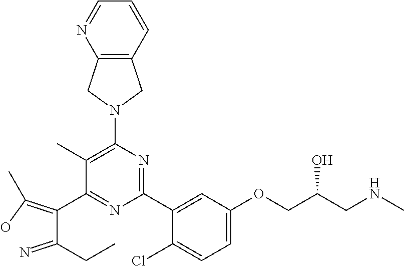 [SMILES: CCC(=N)/C(=C(/C)O)c1nc(-c2cc(OC[C@H](O)CNC)ccc2Cl)nc(N2Cc3cccnc3C2)c1C] | 0.0046 | 0.134 | 535.2 |
| 227 | 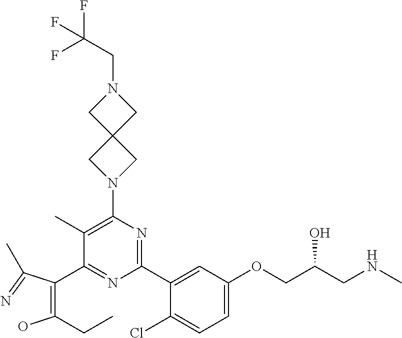 [SMILES: CC/C(O)=C(/C(C)=N)c1nc(-c2cc(OC[C@H](O)CNC)ccc2Cl)nc(N2CC3(CN(CC(F)(F)F)C3)C2)c1C] | 0.0080 | 0.059 | 595.2 |
| 228 | 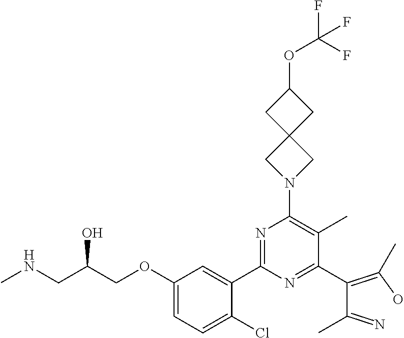 [SMILES: CNC[C@@H](O)COc1ccc(Cl)c(-c2nc(/C(C(C)=N)=C(\C)O)c(C)c(N3CC4(CC(OC(F)(F)F)C4)C3)n2)c1] | 0.0047 | 0.073 | 582.1 |
| 229 | 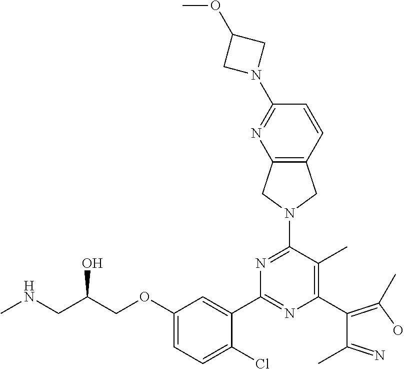 [SMILES: CNC[C@@H](O)COc1ccc(Cl)c(-c2nc(/C(C(C)=N)=C(\C)O)c(C)c(N3Cc4ccc(N5CC(OC)C5)nc4C3)n2)c1] | 0.0019 | 0.014 | 606.2 |
| 230 | 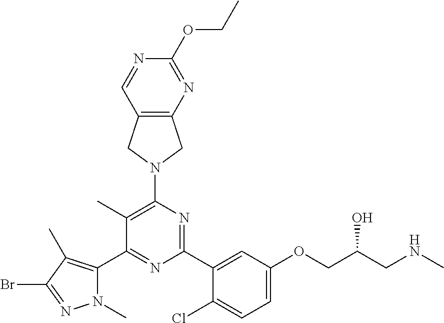 [SMILES: CCOc1ncc2c(n1)CN(c1nc(-c3cc(OC[C@H](O)CNC)ccc3Cl)nc(-c3c(C)c(Br)nn3C)c1C)C2] | 0.0018 | 0.017 | 469.1 |
| 231 | 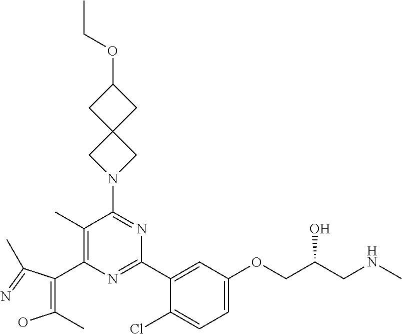 [SMILES: CCOC1CC2(C1)CN(c1nc(-c3cc(OC[C@H](O)CNC)ccc3Cl)nc(/C(C(C)=N)=C(\C)O)c1C)C2] | 0.0046 | 0.041 | 542.1 |
| 232 | 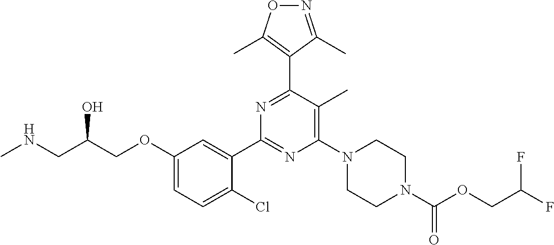 [SMILES: CNC[C@@H](O)COc1ccc(Cl)c(-c2nc(-c3c(C)noc3C)c(C)c(N3CCN(C(=O)OCC(F)F)CC3)n2)c1] | 0.0049 | 0.348 | 595 |
| 233 | 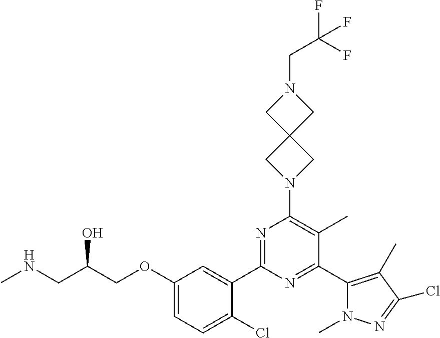 [SMILES: CNC[C@@H](O)COc1ccc(Cl)c(-c2nc(-c3c(C)c(Cl)nn3C)c(C)c(N3CC4(CN(CC(F)(F)F)C4)C3)n2)c1] | 0.0043 | 0.029 | 614 |
| 234 | 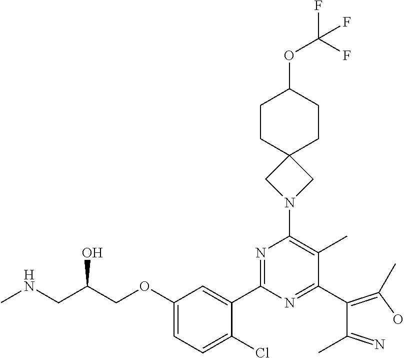 [SMILES: CNC[C@@H](O)COc1ccc(Cl)c(-c2nc(/C(C(C)=N)=C(\C)O)c(C)c(N3CC4(CCC(OC(F)(F)F)CC4)C3)n2)c1] | 0.0061 | 0.105 | 610 |
| 235 | 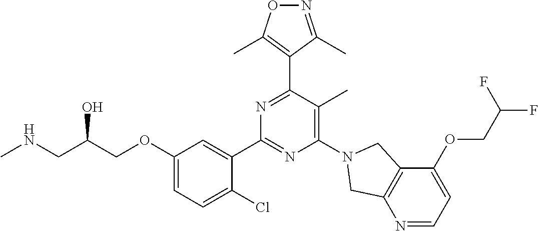 [SMILES: CNC[C@@H](O)COc1ccc(Cl)c(-c2nc(-c3c(C)noc3C)c(C)c(N3Cc4nccc(OCC(F)F)c4C3)n2)c1] | 0.0007 | 0.054 | 601.2 |
| 236 | 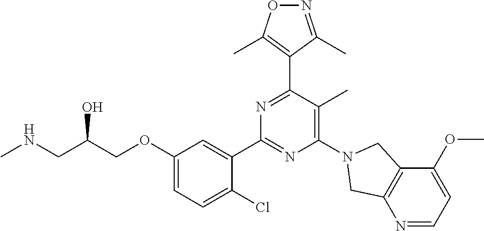 | 0.0018 | 0.063 | 551 |
| 237 | 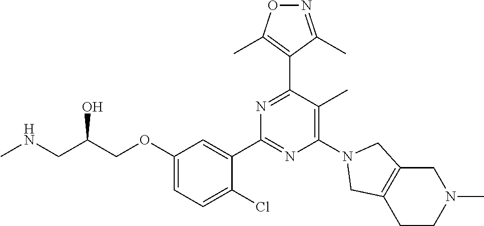 | 0.0006 | 0.157 | 539.2 |
| 238 | 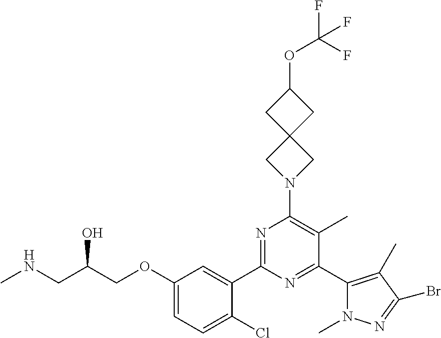 [SMILES: CNC[C@@H](O)COc1ccc(Cl)c(-c2nc(-c3c(C)c(Br)nn3C)c(C)c(N3CC4(CC(OC(F)(F)F)C4)C3)n2)c1] | 0.0050 | 0.116 | 660.8 |
| 239 | 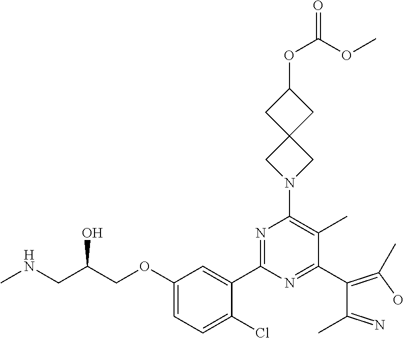 [SMILES: CNC[C@@H](O)COc1ccc(Cl)c(-c2nc(/C(C(C)=N)=C(\C)O)c(C)c(N3CC4(CC(OC(=O)OC)C4)C3)n2)c1] | 0.0047 | 0.052 | 572.3 |
| 240 | 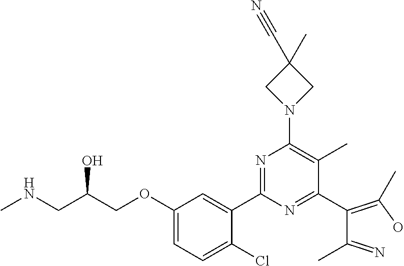 [SMILES: CNC[C@@H](O)COc1ccc(Cl)c(-c2nc(/C(C(C)=N)=C(\C)O)c(C)c(N3CC(C)(C#N)C3)n2)c1] | 0.0041 | 0.398 | 497 |
| 241 | 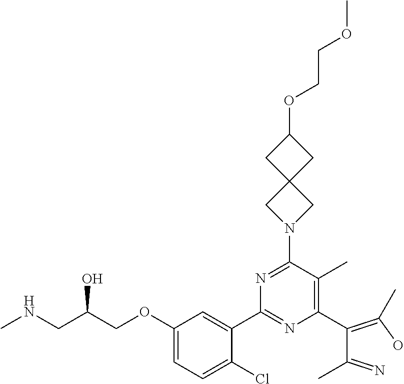 [SMILES: CNC[C@@H](O)COc1ccc(Cl)c(-c2nc(/C(C(C)=N)=C(\C)O)c(C)c(N3CC4(CC(OCCOC)C4)C3)n2)c1] | 0.0062 | 0.084 | 572 |
| 242 | 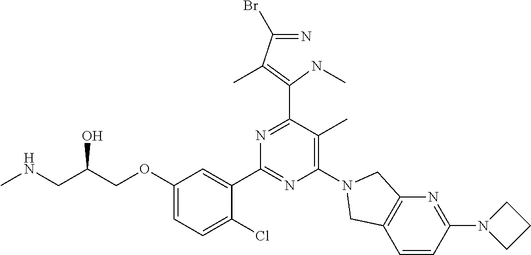 [SMILES: CNC[C@@H](O)COc1ccc(Cl)c(-c2nc(/C(NC)=C(\C)C(=N)Br)c(C)c(N3Cc4ccc(N5CCC5)nc4C3)n2)c1] | 0.0012 | 0.030 | 655 |
| 243 | 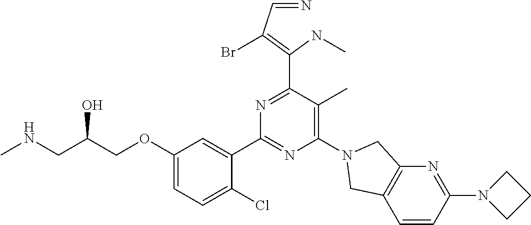 [SMILES: CNC[C@@H](O)COc1ccc(Cl)c(-c2nc(/C(NC)=C(\Br)C=N)c(C)c(N3Cc4ccc(N5CCC5)nc4C3)n2)c1] | 0.0008 | 0.037 | 638.9 |
| 244 | 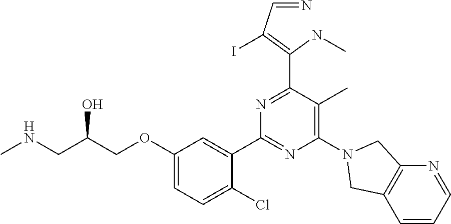 [SMILES: CNC[C@@H](O)COc1ccc(Cl)c(-c2nc(/C(NC)=C(\I)C=N)c(C)c(N3Cc4cccnc4C3)n2)c1] | 0.0012 | 0.141 | 632.1 |
| 245 | 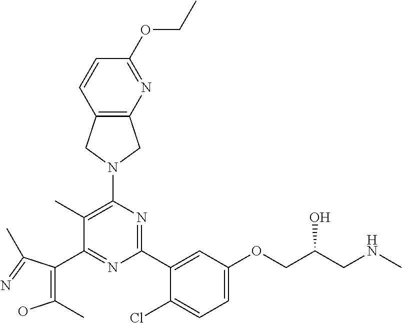 [SMILES: CCOc1ccc2c(n1)CN(c1nc(-c3cc(OC[C@H](O)CNC)ccc3Cl)nc(/C(C(C)=N)=C(\C)O)c1C)C2] | 0.0020 | 0.038 | 565.2 |
| 246 | 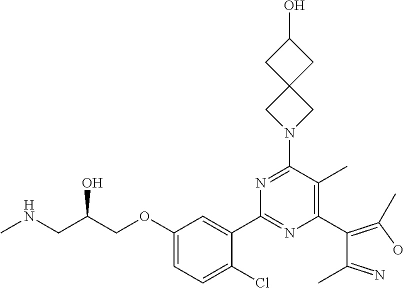 [SMILES: CNC[C@@H](O)COc1ccc(Cl)c(-c2nc(/C(C(C)=N)=C(\C)O)c(C)c(N3CC4(CC(O)C4)C3)n2)c1] | 0.0067 | 0.352 | 514 |
| 247 | 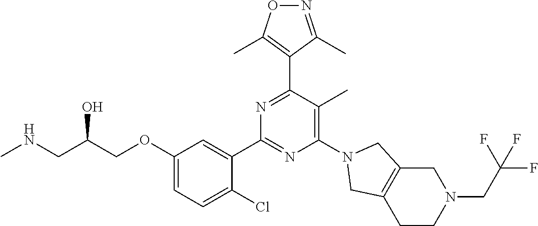 [SMILES: CNC[C@@H](O)COc1ccc(Cl)c(-c2nc(-c3c(C)noc3C)c(C)c(N3CC4=C(CN(CC(F)(F)F)CC4)C3)n2)c1] | 0.0040 | 0.062 | 607.2 |
| 248 | 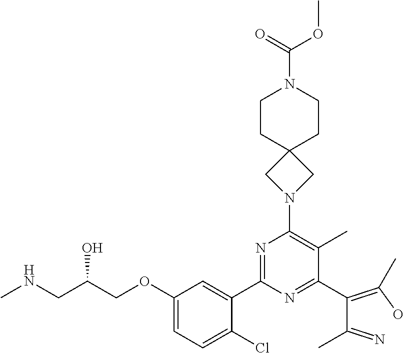 [SMILES: CNC[C@H](O)COc1ccc(Cl)c(-c2nc(/C(C(C)=N)=C(\C)O)c(C)c(N3CC4(CCN(C(=O)OC)CC4)C3)n2)c1] | 0.0058 | 0.294 | 585.3 |
| 249 | 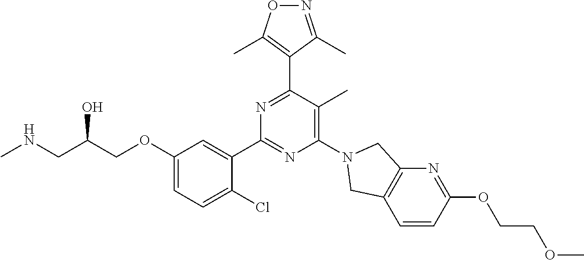 | 0.0020 | 0.027 | 595.2 |
| 250 | 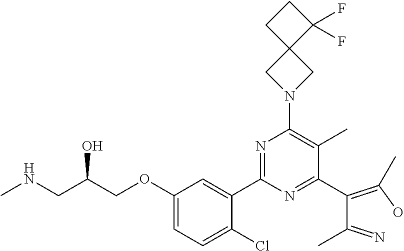 [SMILES: CNC[C@@H](O)COc1ccc(Cl)c(-c2nc(/C(C(C)=N)=C(\C)O)c(C)c(N3CC4(CCC4(F)F)C3)n2)c1] | 0.00394 | 0.21705 | 534 |
| 251 | 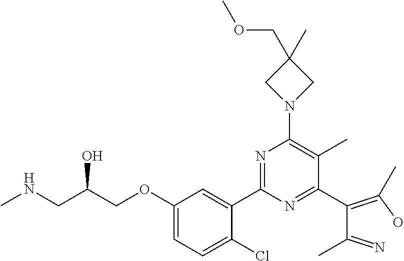 [SMILES: CNC[C@@H](O)COc1ccc(Cl)c(-c2nc(/C(C(C)=N)=C(\C)O)c(C)c(N3CC(C)(COC)C3)n2)c1] | 0.00651 | 0.19256 | 516.2 |
| 252 | 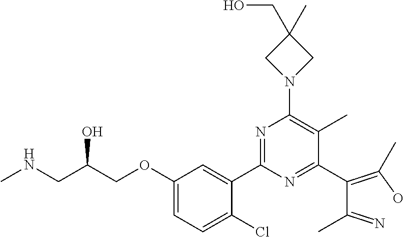 [SMILES: CNC[C@@H](O)COc1ccc(Cl)c(-c2nc(/C(C(C)=N)=C(\C)O)c(C)c(N3CC(C)(CO)C3)n2)c1] | 0.00801 | 0.56093 | 502 |
| 253 | 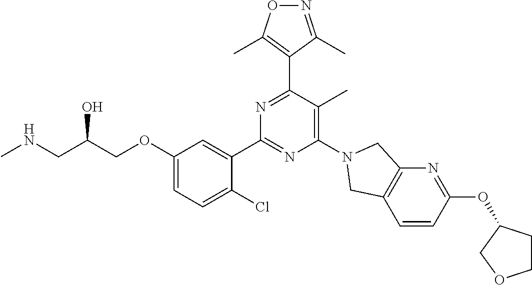 [SMILES: CNC[C@@H](O)COc1ccc(Cl)c(-c2nc(-c3c(C)noc3C)c(C)c(N3Cc4ccc(O[C@@H]5CCOC5)nc4C3)n2)c1] | 0.00236 | 0.02919 | 607.2 |
| 254 | 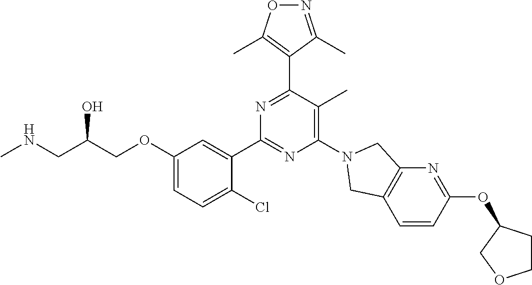 [SMILES: CNC[C@@H](O)COc1ccc(Cl)c(-c2nc(-c3c(C)noc3C)c(C)c(N3Cc4ccc(O[C@H]5CCOC5)nc4C3)n2)c1] | 0.00239 | 0.03713 | 607.1 |
| 255 | 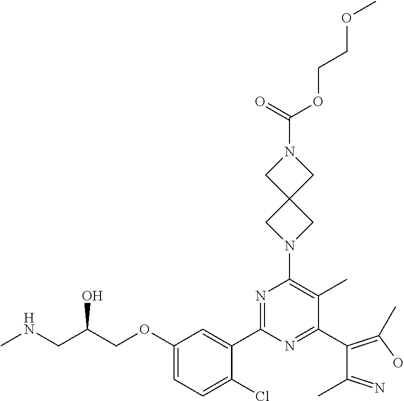 [SMILES: CNC[C@@H](O)COc1ccc(Cl)c(-c2nc(/C(C(C)=N)=C(\C)O)c(C)c(N3CC4(CN(C(=O)OCCOC)C4)C3)n2)c1] | 0.00722 | 0.37722 | 601 |
| 256 | 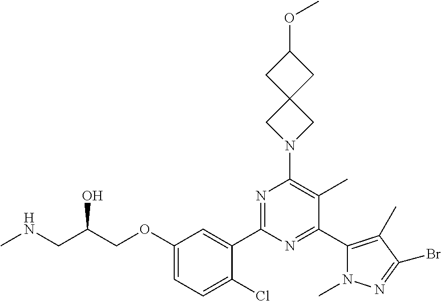 [SMILES: CNC[C@@H](O)COc1ccc(Cl)c(-c2nc(-c3c(C)c(Br)nn3C)c(C)c(N3CC4(CC(OC)C4)C3)n2)c1] | 0.00396 | 0.04322 | 605.2 |
| 257 | 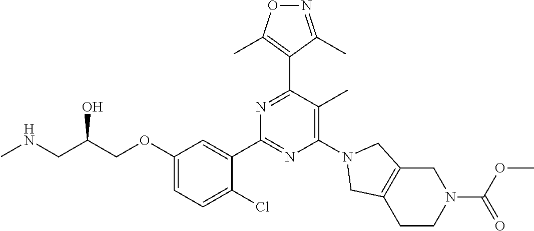 | 0.0024 | 0.02268 | 583.2 |
| 258 | 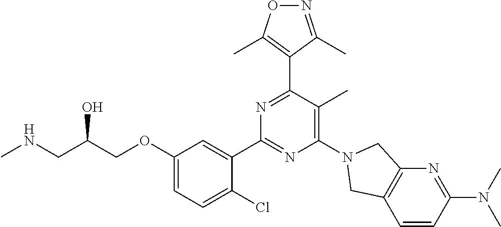 | 0.00183 | 0.02985 | 564 |
| 259 | 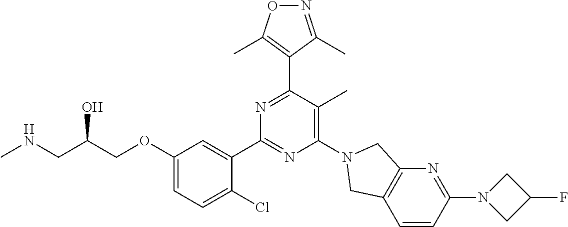 [SMILES: CNC[C@@H](O)COc1ccc(Cl)c(-c2nc(-c3c(C)noc3C)c(C)c(N3Cc4ccc(N5CC(F)C5)nc4C3)n2)c1] | 0.00217 | 0.0229 | 594.1 |
| 260 | 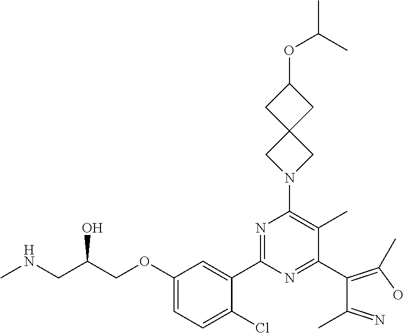 [SMILES: CNC[C@@H](O)COc1ccc(Cl)c(-c2nc(/C(C(C)=N)=C(\C)O)c(C)c(N3CC4(CC(OC(C)C)C4)C3)n2)c1] | 0.00595 | 0.04546 | 556.2 |
| 261 | 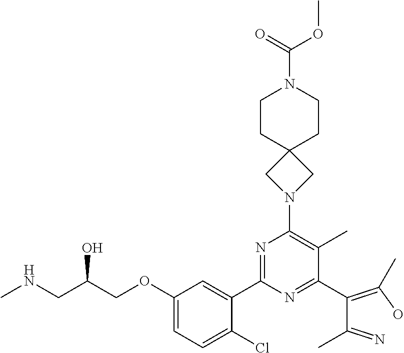 [SMILES: CNC[C@@H](O)COc1ccc(Cl)c(-c2nc(/C(C(C)=N)=C(\C)O)c(C)c(N3CC4(CCN(C(=O)OC)CC4)C3)n2)c1] | 0.00401 | 0.05391 | 585.2 |
| 262 | 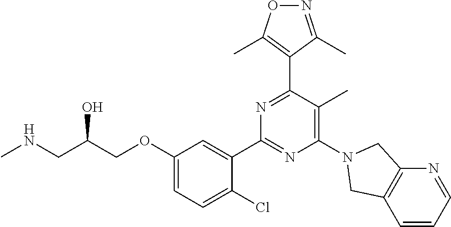 [SMILES: CNC[C@@H](O)COc1ccc(Cl)c(-c2nc(-c3c(C)noc3C)c(C)c(N3Cc4cccnc4C3)n2)c1] | 0.00234 | 0.03257 | 521 |
| 263 | 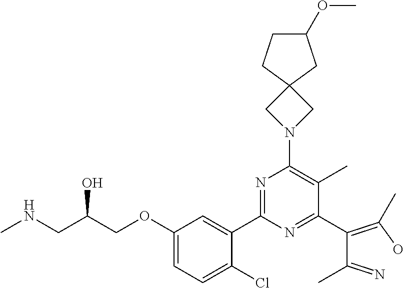 [SMILES: CNC[C@@H](O)COc1ccc(Cl)c(-c2nc(/C(C(C)=N)=C(\C)O)c(C)c(N3CC4(CCC(OC)C4)C3)n2)c1] | 0.00536 | 0.09086 | 542 |
| 264 | 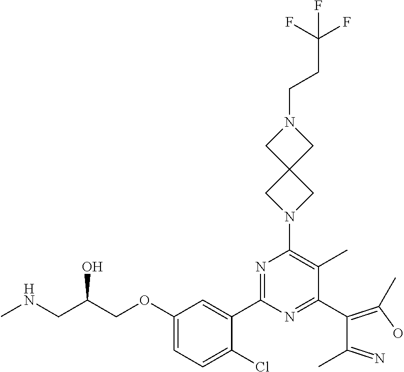 [SMILES: CNC[C@@H](O)COc1ccc(Cl)c(-c2nc(/C(C(C)=N)=C(\C)O)c(C)c(N3CC4(CN(CCC(F)(F)F)C4)C3)n2)c1] | 0.00411 | 0.05187 | 595 |
| 265 | 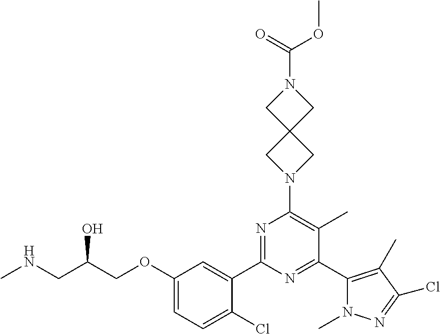 [SMILES: CNC[C@@H](O)COc1ccc(Cl)c(-c2nc(-c3c(C)c(Cl)nn3C)c(C)c(N3CC4(CN(C(=O)OC)C4)C3)n2)c1] | 0.0042 | 0.08773 | 590 |
| 266 | 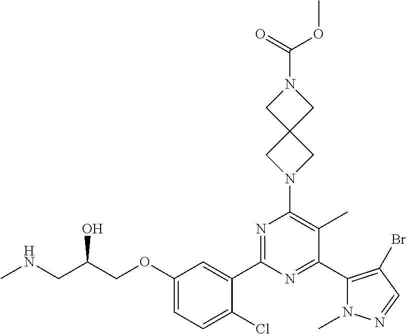 [SMILES: CNC[C@@H](O)COc1ccc(Cl)c(-c2nc(-c3c(Br)cnn3C)c(C)c(N3CC4(CN(C(=O)OC)C4)C3)n2)c1] | 0.00683 | 0.30986 | 622 |
| 267 | 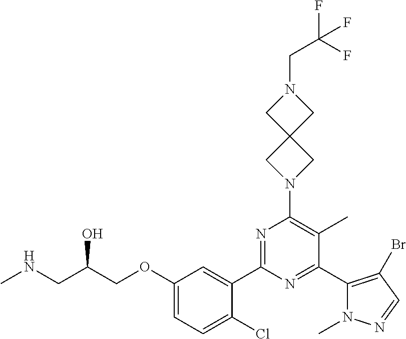 [SMILES: CNC[C@@H](O)COc1ccc(Cl)c(-c2nc(-c3c(Br)cnn3C)c(C)c(N3CC4(CN(CC(F)(F)F)C4)C3)n2)c1] | 0.00897 | 0.1208 | 645.9 |
| 268 | 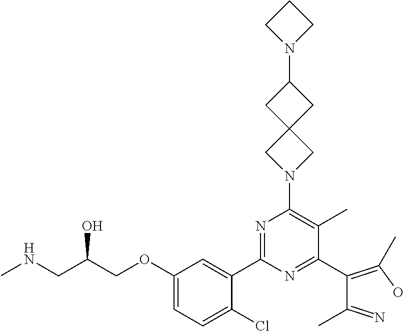 [SMILES: CNC[C@@H](O)COc1ccc(Cl)c(-c2nc(/C(C(C)=N)=C(\C)O)c(C)c(N3CC4(CC(N5CCC5)C4)C3)n2)c1] | 0.00126 | 1.35264 | 553 |
| 269 | 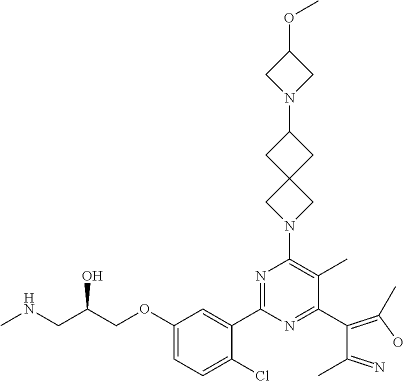 [SMILES: CNC[C@@H](O)COc1ccc(Cl)c(-c2nc(/C(C(C)=N)=C(\C)O)c(C)c(N3CC4(CC(N5CC(OC)C5)C4)C3)n2)c1] | 0.0008 | 0.35455 | 583 |
| 270 | 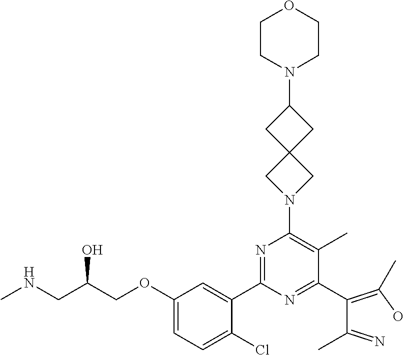 [SMILES: CNC[C@@H](O)COc1ccc(Cl)c(-c2nc(/C(C(C)=N)=C(\C)O)c(C)c(N3CC4(CC(N5CCOCC5)C4)C3)n2)c1] | 0.00495 | 0.26902 | 583 |
| 271 | 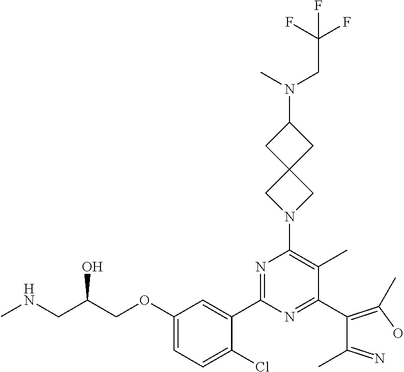 [SMILES: CNC[C@@H](O)COc1ccc(Cl)c(-c2nc(/C(C(C)=N)=C(\C)O)c(C)c(N3CC4(CC(N(C)CC(F)(F)F)C4)C3)n2)c1] | 0.00742 | 0.08906 | 609.2 |
| 272 | 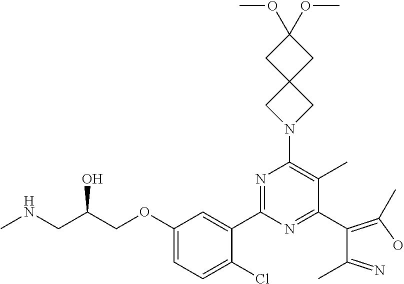 [SMILES: CNC[C@@H](O)COc1ccc(Cl)c(-c2nc(/C(C(C)=N)=C(\C)O)c(C)c(N3CC4(C3)CC(OC)(OC)C4)n2)c1] | 0.00543 | 0.07897 | 558.2 |
| 273 | 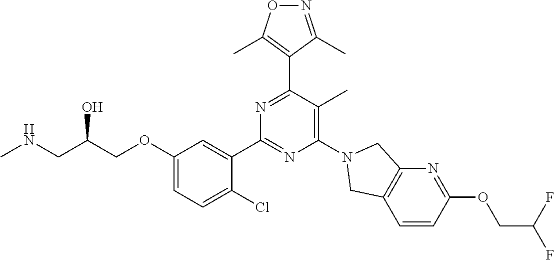 [SMILES: CNC[C@@H](O)COc1ccc(Cl)c(-c2nc(-c3c(C)noc3C)c(C)c(N3Cc4ccc(OCC(F)F)nc4C3)n2)c1] | 0.00226 | 0.06192 | 601 |
| 274 | 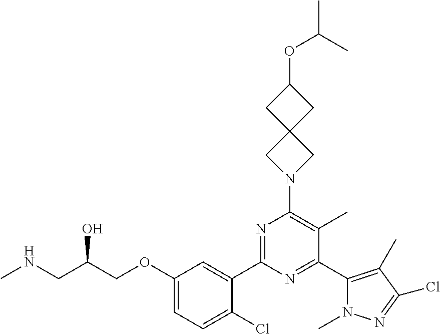 [SMILES: CNC[C@@H](O)COc1ccc(Cl)c(-c2nc(-c3c(C)c(Cl)nn3C)c(C)c(N3CC4(CC(OC(C)C)C4)C3)n2)c1] | 0.00503 | 0.04876 | 589 |
| 275 | 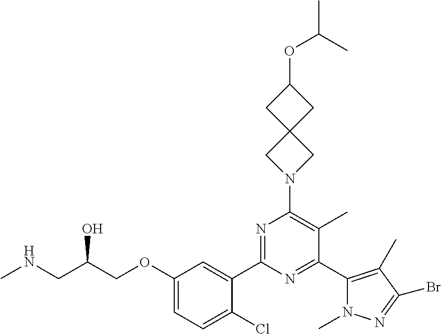 [SMILES: CNC[C@@H](O)COc1ccc(Cl)c(-c2nc(-c3c(C)c(Br)nn3C)c(C)c(N3CC4(CC(OC(C)C)C4)C3)n2)c1] | 0.00353 | 0.04499 | 634.9 |
| 276 | 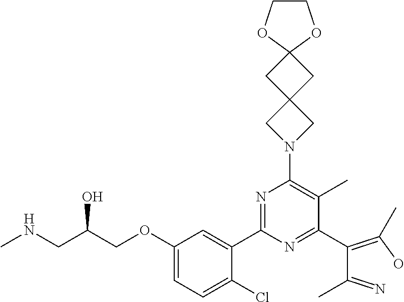 [SMILES: CNC[C@@H](O)COc1ccc(Cl)c(-c2nc(/C(C(C)=N)=C(\C)O)c(C)c(N3CC4(C3)CC3(C4)OCCO3)n2)c1] | 0.00492 | 0.07573 | 556 |
| 277 | 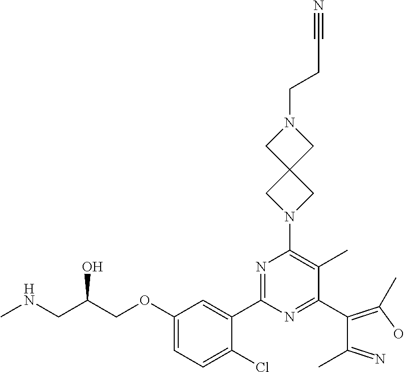 [SMILES: CNC[C@@H](O)COc1ccc(Cl)c(-c2nc(/C(C(C)=N)=C(\C)O)c(C)c(N3CC4(CN(CCC#N)C4)C3)n2)c1] | 0.00824 | 1.46961 | 552 |
| 278 | 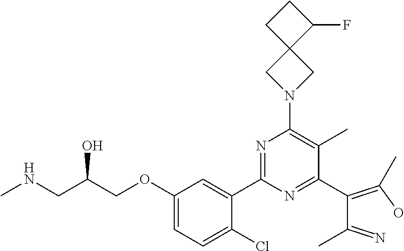 [SMILES: CNC[C@@H](O)COc1ccc(Cl)c(-c2nc(/C(C(C)=N)=C(\C)O)c(C)c(N3CC4(CCC4F)C3)n2)c1] | 0.00411 | 0.5443 | 516.3 |
| 279 | 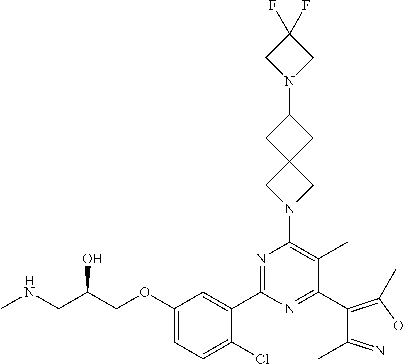 [SMILES: CNC[C@@H](O)COc1ccc(Cl)c(-c2nc(/C(C(C)=N)=C(\C)O)c(C)c(N3CC4(CC(N5CC(F)(F)C5)C4)C3)n2)c1] | 0.00575 | 0.05421 | 589.2 |
| 280 | 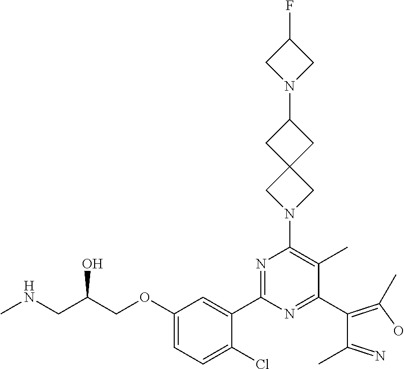 [SMILES: CNC[C@@H](O)COc1ccc(Cl)c(-c2nc(/C(C(C)=N)=C(\C)O)c(C)c(N3CC4(CC(N5CC(F)C5)C4)C3)n2)c1] | 0.00209 | 0.10456 | 571.2 |
| 281 | 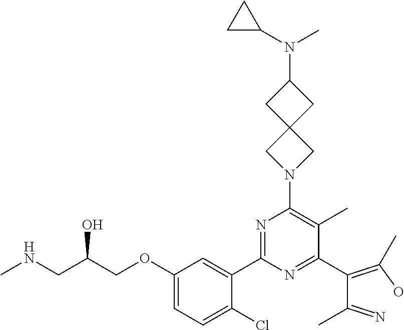 [SMILES: CNC[C@@H](O)COc1ccc(Cl)c(-c2nc(/C(C(C)=N)=C(\C)O)c(C)c(N3CC4(CC(N(C)C5CC5)C4)C3)n2)c1] | 0.00104 | 0.0792 | 567.1 |
| 282 | 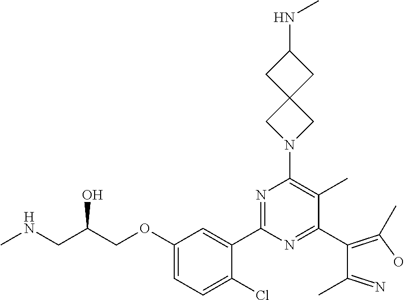 [SMILES: CNC[C@@H](O)COc1ccc(Cl)c(-c2nc(/C(C(C)=N)=C(\C)O)c(C)c(N3CC4(CC(NC)C4)C3)n2)c1] | 0.0009 | 2.21372 | 527 |
| 283 | 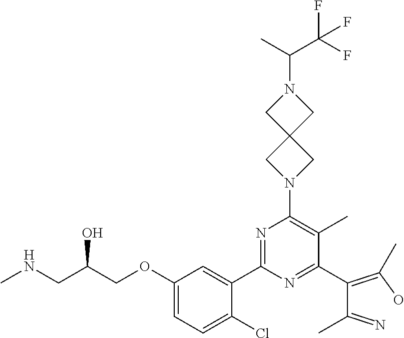 [SMILES: CNC[C@@H](O)COc1ccc(Cl)c(-c2nc(/C(C(C)=N)=C(\C)O)c(C)c(N3CC4(C3)CN(C(C)C(F)(F)F)C4)n2)c1] | 0.00567 | 0.04841 | 595.2 |
| 284 | 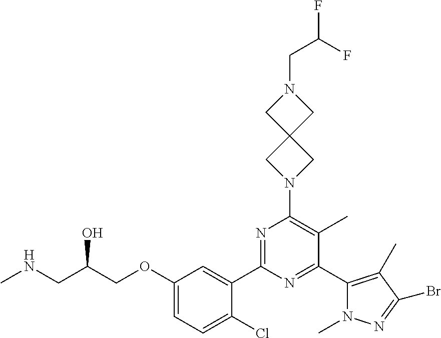 [SMILES: CNC[C@@H](O)COc1ccc(Cl)c(-c2nc(-c3c(C)c(Br)nn3C)c(C)c(N3CC4(CN(CC(F)F)C4)C3)n2)c1] | 0.00236 | 0.03916 | 642 |
| 285 | 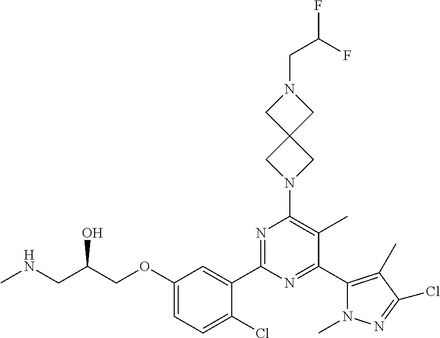 [SMILES: CNC[C@@H](O)COc1ccc(Cl)c(-c2nc(-c3c(C)c(Cl)nn3C)c(C)c(N3CC4(CN(CC(F)F)C4)C3)n2)c1] | 0.0024 | 0.04607 | 596 |
| 286 | 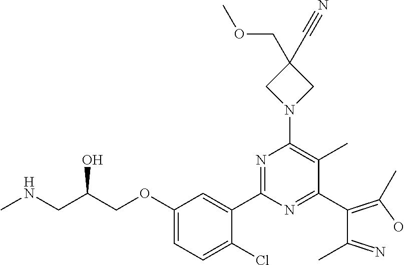 [SMILES: CNC[C@@H](O)COc1ccc(Cl)c(-c2nc(/C(C(C)=N)=C(\C)O)c(C)c(N3CC(C#N)(COC)C3)n2)c1] | 0.00417 | 0.28207 | 527.3 |
| 287 | 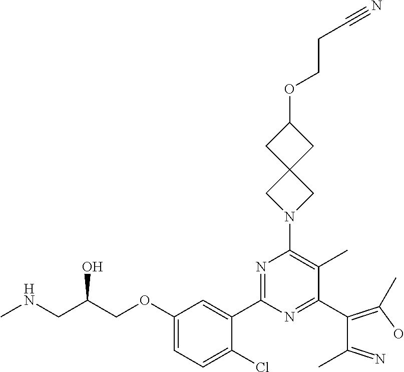 [SMILES: CNC[C@@H](O)COc1ccc(Cl)c(-c2nc(/C(C(C)=N)=C(\C)O)c(C)c(N3CC4(CC(OCCC#N)C4)C3)n2)c1] | 0.00446 | 0.06909 | 567.2 |
| 288 | 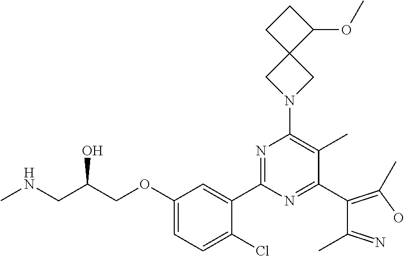 [SMILES: CNC[C@@H](O)COc1ccc(Cl)c(-c2nc(/C(C(C)=N)=C(\C)O)c(C)c(N3CC4(CCC4OC)C3)n2)c1] | 0.00386 | 0.23408 | 528.2 |
| 289 | 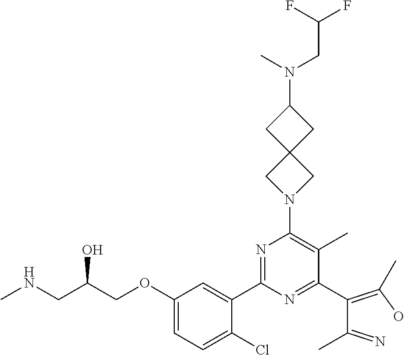 [SMILES: CNC[C@@H](O)COc1ccc(Cl)c(-c2nc(/C(C(C)=N)=C(\C)O)c(C)c(N3CC4(CC(N(C)CC(F)F)C4)C3)n2)c1] | 0.00408 | 0.07595 | 591 |
| 290 | 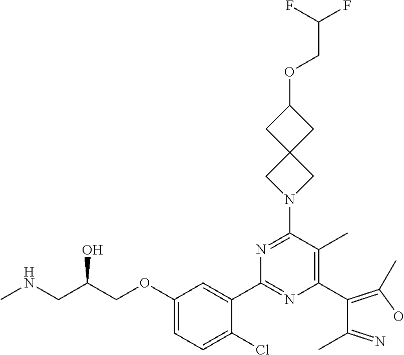 [SMILES: CNC[C@@H](O)COc1ccc(Cl)c(-c2nc(/C(C(C)=N)=C(\C)O)c(C)c(N3CC4(CC(OCC(F)F)C4)C3)n2)c1] | 0.00339 | 0.03824 | 578.2 |
| 291 | 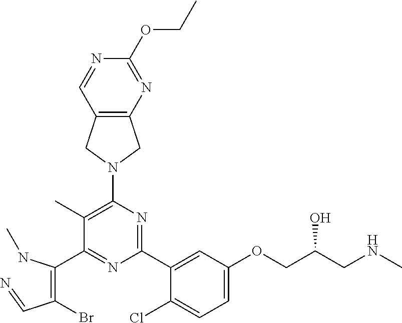 [SMILES: CCOc1ncc2c(n1)CN(c1nc(-c3cc(OC[C@H](O)CNC)ccc3Cl)nc(/C(NC)=C(\Br)C=N)c1C)C2] | 0.002 | 0.0368 | 630.8 |
| 292 | 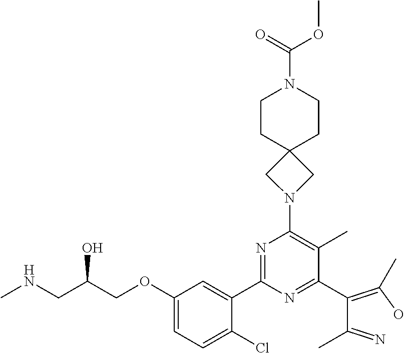 [SMILES: CNC[C@@H](O)COc1ccc(Cl)c(-c2nc(/C(C(C)=N)=C(\C)O)c(C)c(N3CC4(CCN(C(=O)OC)CC4)C3)n2)c1] | 0.00349 | 0.06671 | 585.3 |
| 293 | 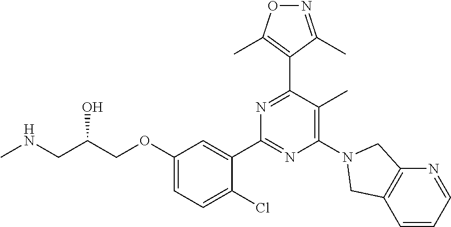 [SMILES: CNC[C@H](O)COc1ccc(Cl)c(-c2nc(-c3c(C)noc3C)c(C)c(N3Cc4cccnc4C3)n2)c1] | 0.00142 | 0.1549 | 521 |
| 294 | 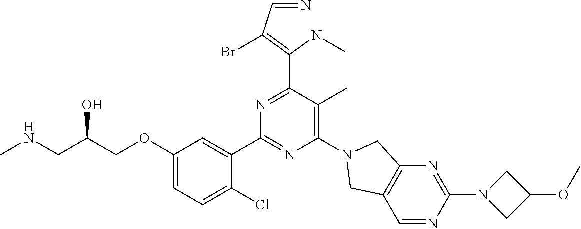 | 0.00117 | 0.01624 | 671.9 |
| 295 | 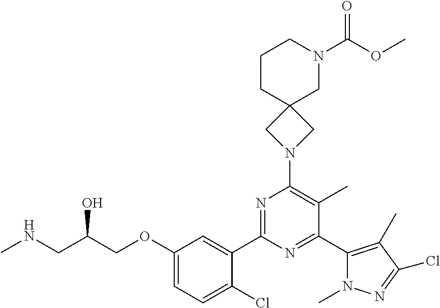 [SMILES: CNC[C@@H](O)COc1ccc(Cl)c(-c2nc(-c3c(C)c(Cl)nn3C)c(C)c(N3CC4(CCCN(C(=O)OC)C4)C3)n2)c1] | 0.00378 | 0.08272 | 618 |
| 296 | 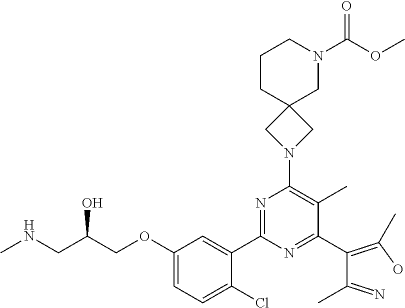 | 0.0064 | 0.137 | 585 |
| 297 | 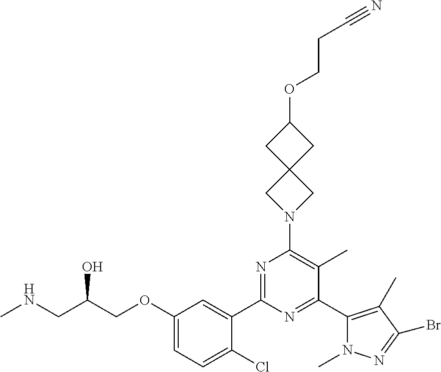 [SMILES: CNC[C@@H](O)COc1ccc(Cl)c(-c2nc(-c3c(C)c(Br)nn3C)c(C)c(N3CC4(CC(OCCC#N)C4)C3)n2)c1] | 0.00283 | 0.03938 | 646 |
| 298 | 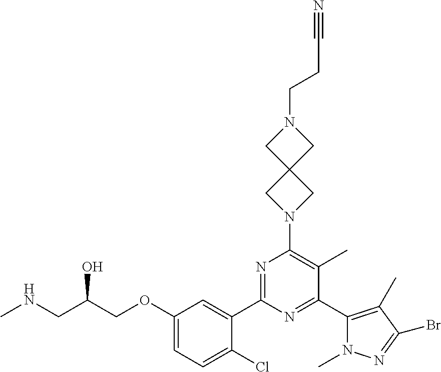 [SMILES: CNC[C@@H](O)COc1ccc(Cl)c(-c2nc(-c3c(C)c(Br)nn3C)c(C)c(N3CC4(CN(CCC#N)C4)C3)n2)c1] | 0.00311 | 0.24435 | 630.9 |
| 299 | 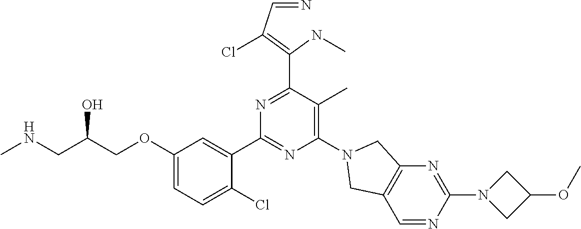 | 0.00294 | 0.01758 | 626 |
| 300 | 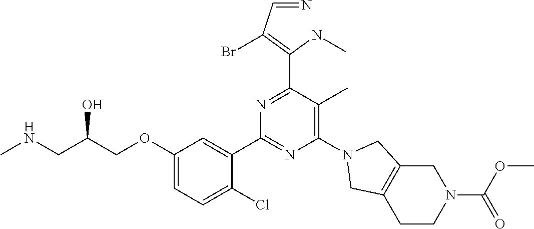 | 0.00476 | 0.0423 | 648.1 |
| 301 | 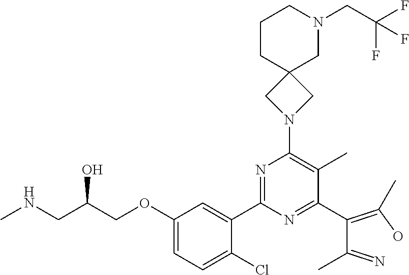 [SMILES: CNC[C@@H](O)COc1ccc(Cl)c(-c2nc(/C(C(C)=N)=C(\C)O)c(C)c(N3CC4(CCCN(CC(F)(F)F)C4)C3)n2)c1] | 0.00799 | 0.12512 | 609.2 |
| 302 | 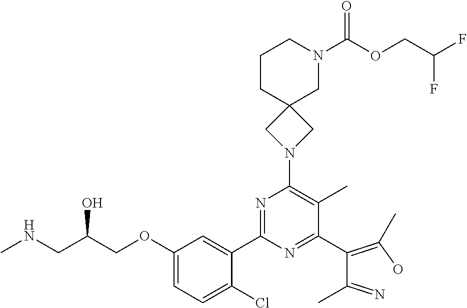 [SMILES: CNC[C@@H](O)COc1ccc(Cl)c(-c2nc(/C(C(C)=N)=C(\C)O)c(C)c(N3CC4(CCCN(C(=O)OCC(F)F)C4)C3)n2)c1] | 0.00518 | 0.07024 | 635 |
| 303 | 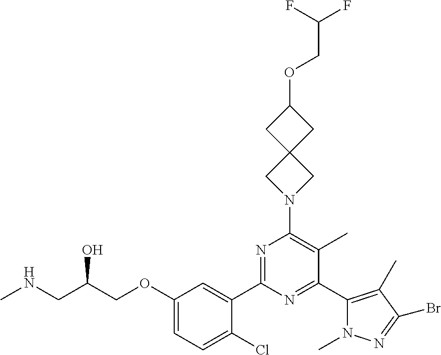 [SMILES: CNC[C@@H](O)COc1ccc(Cl)c(-c2nc(-c3c(C)c(Br)nn3C)c(C)c(N3CC4(CC(OCC(F)F)C4)C3)n2)c1] | 0.00591 | 0.03759 | 657 |
| 304 | 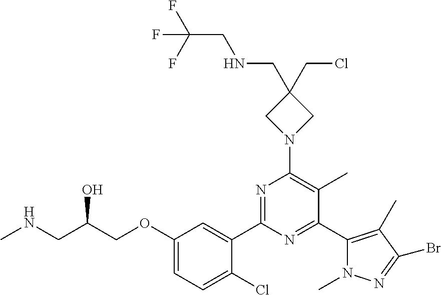 [SMILES: CNC[C@@H](O)COc1ccc(Cl)c(-c2nc(-c3c(C)c(Br)nn3C)c(C)c(N3CC(CCl)(CNCC(F)(F)F)C3)n2)c1] | 0.00681 | 0.14155 | 695.8 |
| 305 | 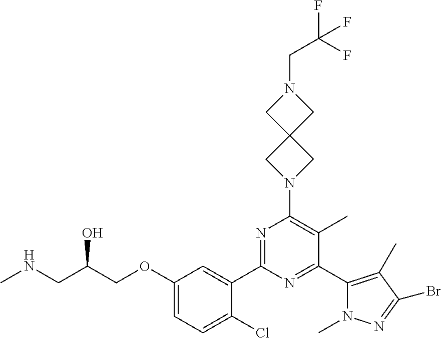 [SMILES: CNC[C@@H](O)COc1ccc(Cl)c(-c2nc(-c3c(C)c(Br)nn3C)c(C)c(N3CC4(CN(CC(F)(F)F)C4)C3)n2)c1] | 0.00835 | 0.02769 | 657.9 |
| 306 | 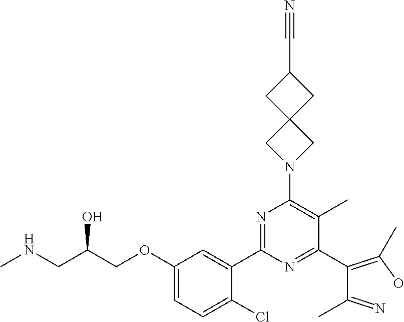 [SMILES: CNC[C@@H](O)COc1ccc(Cl)c(-c2nc(/C(C(C)=N)=C(\C)O)c(C)c(N3CC4(CC(C#N)C4)C3)n2)c1] | 0.00652 | 0.17326 | 523 |
| 307 | 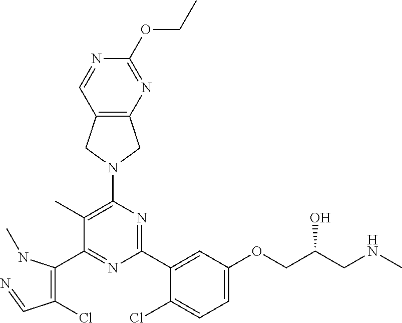 [SMILES: CCOc1ncc2c(n1)CN(c1nc(-c3cc(OC[C@H](O)CNC)ccc3Cl)nc(/C(NC)=C(\Cl)C=N)c1C)C2] | 0.00386 | 0.04542 | 585 |
| 308 | 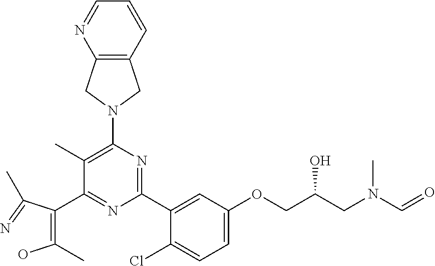 [SMILES: CC(=N)/C(=C(/C)O)c1nc(-c2cc(OC[C@H](O)CN(C)C=O)ccc2Cl)nc(N2Cc3cccnc3C2)c1C] | 0.12031 | 1.81987 | 549.3 |
| 309 | 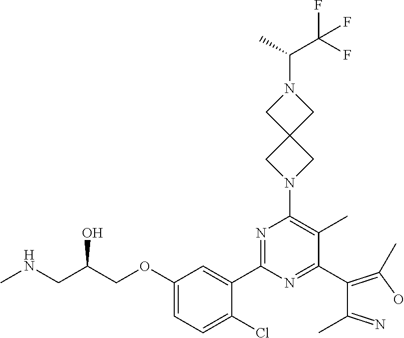 [SMILES: CNC[C@@H](O)COc1ccc(Cl)c(-c2nc(/C(C(C)=N)=C(\C)O)c(C)c(N3CC4(C3)CN([C@H](C)C(F)(F)F)C4)n2)c1] | — | — | 595 |
| 310 | 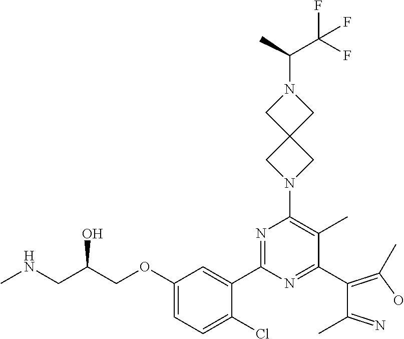 [SMILES: CNC[C@@H](O)COc1ccc(Cl)c(-c2nc(/C(C(C)=N)=C(\C)O)c(C)c(N3CC4(C3)CN([C@@H](C)C(F)(F)F)C4)n2)c1] | — | — | 595 |
| 311 | 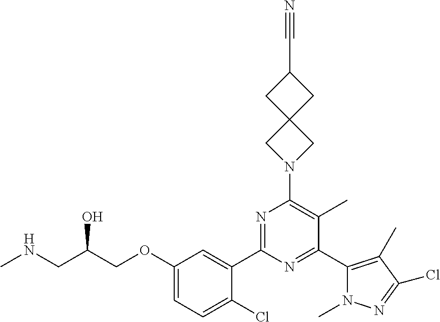 [SMILES: CNC[C@@H](O)COc1ccc(Cl)c(-c2nc(-c3c(C)c(Cl)nn3C)c(C)c(N3CC4(CC(C#N)C4)C3)n2)c1] | — | — | 556 |
| 312 | 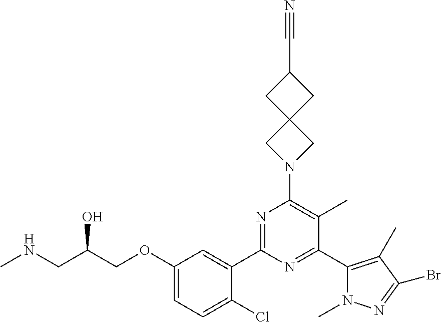 [SMILES: CNC[C@@H](O)COc1ccc(Cl)c(-c2nc(-c3c(C)c(Br)nn3C)c(C)c(N3CC4(CC(C#N)C4)C3)n2)c1] | — | — | 601.9 |
| 313 | 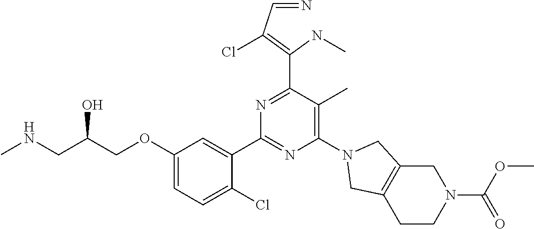 | — | — | 602 |
| 314 | 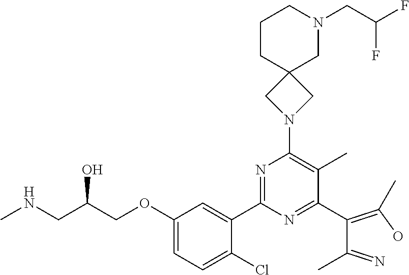 [SMILES: CNC[C@@H](O)COc1ccc(Cl)c(-c2nc(/C(C(C)=N)=C(\C)O)c(C)c(N3CC4(CCCN(CC(F)F)C4)C3)n2)c1] | — | — | — |
| 315 | 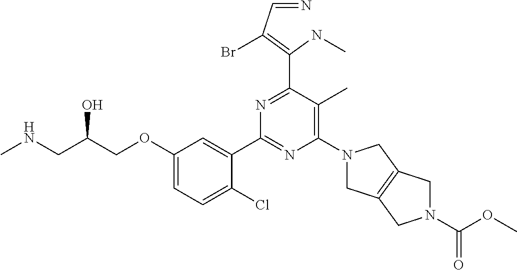 | — | — | — |
| 316 | 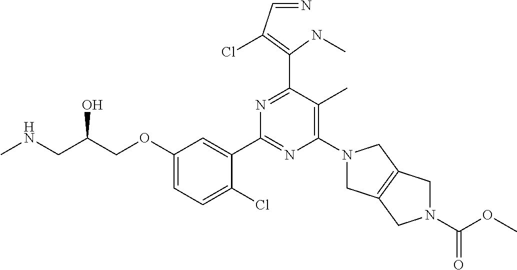 | — | — | — |
| 317 | 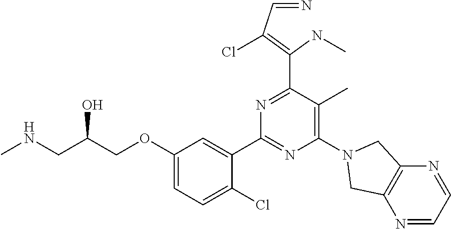 [SMILES: CNC[C@@H](O)COc1ccc(Cl)c(-c2nc(/C(NC)=C(\Cl)C=N)c(C)c(N3Cc4nccnc4C3)n2)c1] | — | — | — |
| 318 | 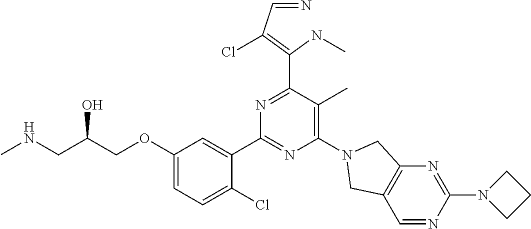 [SMILES: CNC[C@@H](O)COc1ccc(Cl)c(-c2nc(/C(NC)=C(\Cl)C=N)c(C)c(N3Cc4cnc(N5CCC5)nc4C3)n2)c1] | — | — | — |
| 319 | 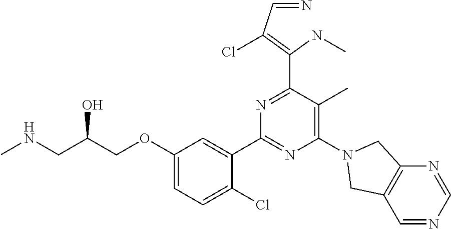 [SMILES: CNC[C@@H](O)COc1ccc(Cl)c(-c2nc(/C(NC)=C(\Cl)C=N)c(C)c(N3Cc4cncnc4C3)n2)c1] | — | — | — |
| 320 | 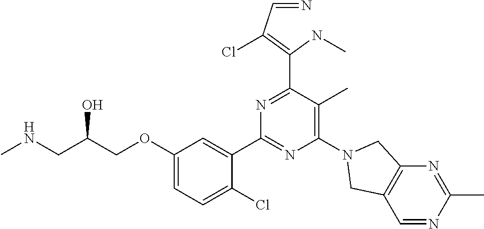 | — | — | — |
| 321 | 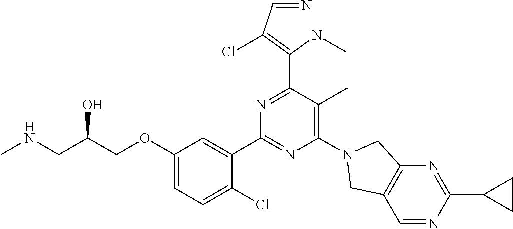 [SMILES: CNC[C@@H](O)COc1ccc(Cl)c(-c2nc(/C(NC)=C(\Cl)C=N)c(C)c(N3Cc4cnc(C5CC5)nc4C3)n2)c1] | — | — | — |
| 322 | 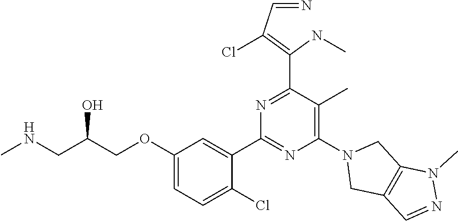 | — | — | — |
| 323 | 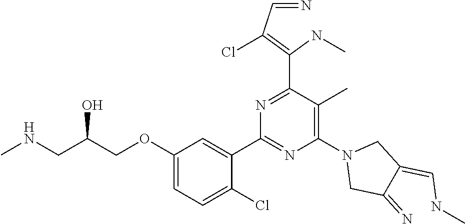 | — | — | — |
| 324 | 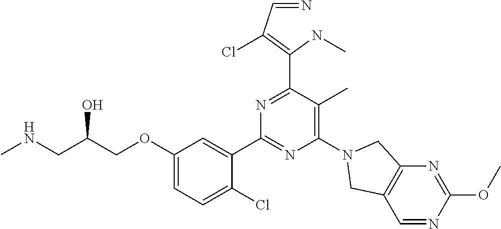 | — | — | — |
| 325 | 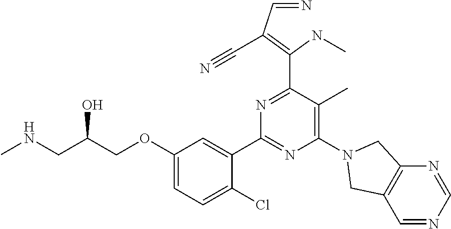 [SMILES: CNC[C@@H](O)COc1ccc(Cl)c(-c2nc(/C(NC)=C(\C#N)C=N)c(C)c(N3Cc4cncnc4C3)n2)c1] | — | — | — |
| 326 | 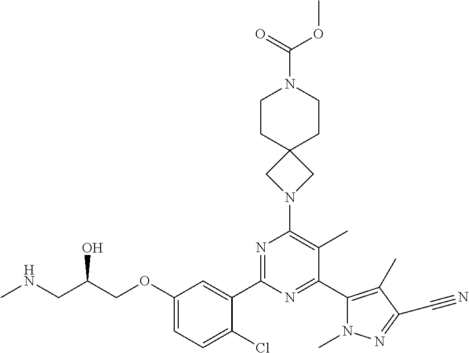 [SMILES: CNC[C@@H](O)COc1ccc(Cl)c(-c2nc(-c3c(C)c(C#N)nn3C)c(C)c(N3CC4(CCN(C(=O)OC)CC4)C3)n2)c1] | — | — | — |
| 327 | 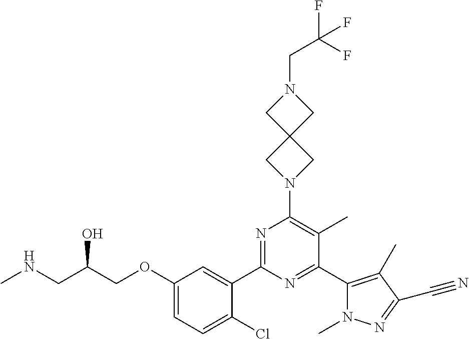 [SMILES: CNC[C@@H](O)COc1ccc(Cl)c(-c2nc(-c3c(C)c(C#N)nn3C)c(C)c(N3CC4(CN(CC(F)(F)F)C4)C3)n2)c1] | — | — | — |
| 328 | 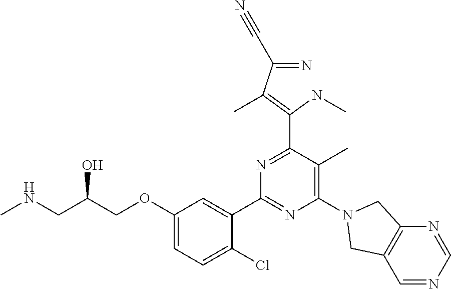 [SMILES: CNC[C@@H](O)COc1ccc(Cl)c(-c2nc(/C(NC)=C(\C)C(=N)C#N)c(C)c(N3Cc4cncnc4C3)n2)c1] | — | — | — |
| 329 | 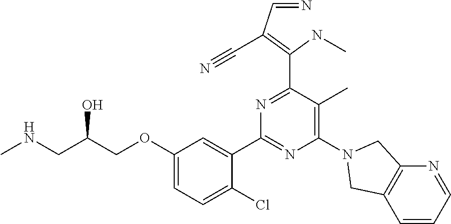 [SMILES: CNC[C@@H](O)COc1ccc(Cl)c(-c2nc(/C(NC)=C(\C#N)C=N)c(C)c(N3Cc4cccnc4C3)n2)c1] | — | — | — |
| 330 | 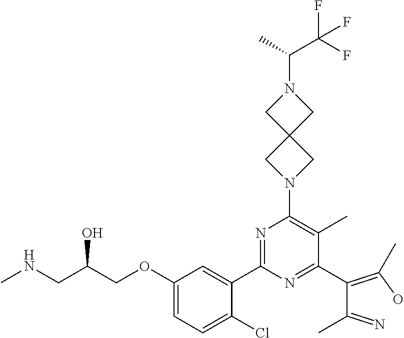 [SMILES: CNC[C@@H](O)COc1ccc(Cl)c(-c2nc(/C(C(C)=N)=C(\C)O)c(C)c(N3CC4(C3)CN([C@H](C)C(F)(F)F)C4)n2)c1] | 0.00483 | 0.04089 | 595 |
| 331 | 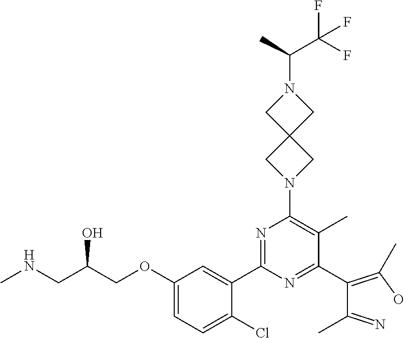 [SMILES: CNC[C@@H](O)COc1ccc(Cl)c(-c2nc(/C(C(C)=N)=C(\C)O)c(C)c(N3CC4(C3)CN([C@@H](C)C(F)(F)F)C4)n2)c1] | 0.00631 | 0.03291 | 595 |
| 332 | 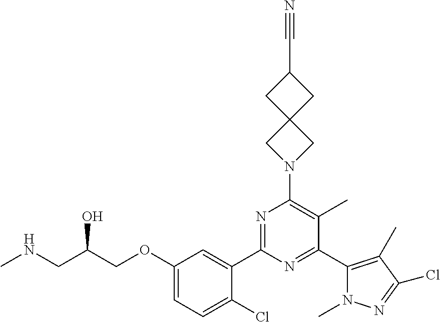 [SMILES: CNC[C@@H](O)COc1ccc(Cl)c(-c2nc(-c3c(C)c(Cl)nn3C)c(C)c(N3CC4(CC(C#N)C4)C3)n2)c1] | 0.0041 | 0.0809 | 556 |
| 333 | 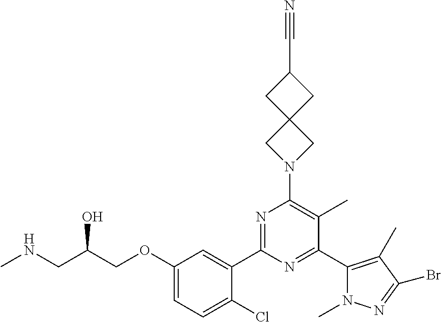 [SMILES: CNC[C@@H](O)COc1ccc(Cl)c(-c2nc(-c3c(C)c(Br)nn3C)c(C)c(N3CC4(CC(C#N)C4)C3)n2)c1] | 0.00246 | 0.06444 | 601.9 |
| 334 | 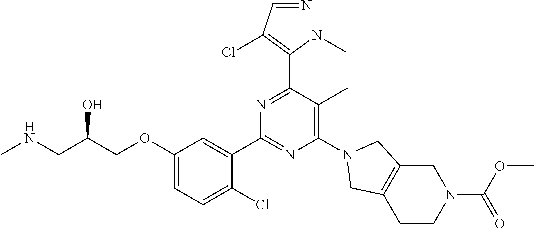 | 0.00253 | 0.0434 | 602 |
| 335 | 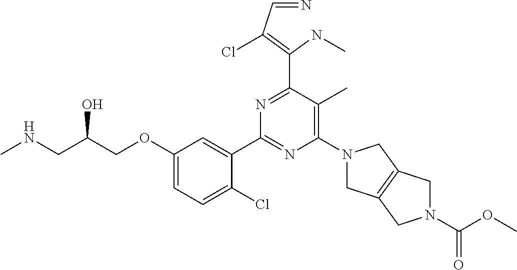 | 0.00228 | 0.03046 | 588 |
| 336 | 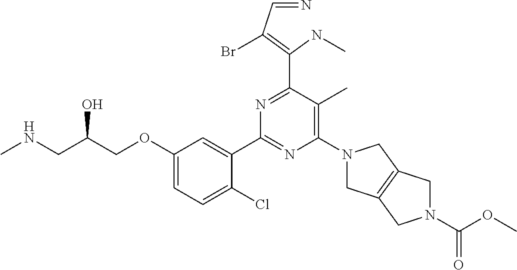 | 0.00303 | 0.03212 | 633.9 |
| 337 | 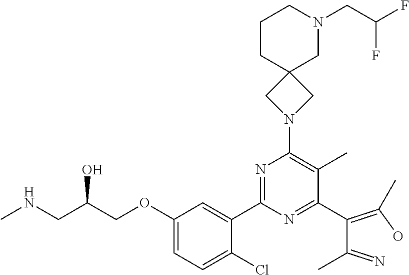 [SMILES: CNC[C@@H](O)COc1ccc(Cl)c(-c2nc(/C(C(C)=N)=C(\C)O)c(C)c(N3CC4(CCCN(CC(F)F)C4)C3)n2)c1] | 0.00498 | 0.05999 | 591 |
| 338 | 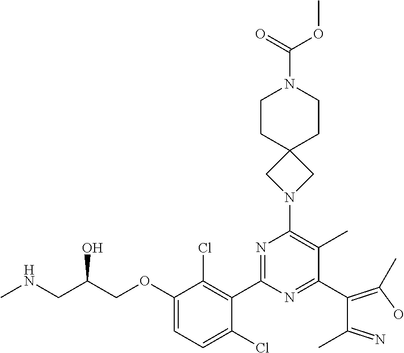 [SMILES: CNC[C@@H](O)COc1ccc(Cl)c(-c2nc(/C(C(C)=N)=C(\C)O)c(C)c(N3CC4(CCN(C(=O)OC)CC4)C3)n2)c1Cl] | 0.03113 | 1.82143 | 619 |
| 339 | 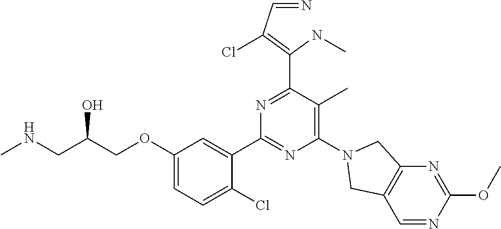 | 0.00254 | 0.06536 | 571.1 |
| 340 | 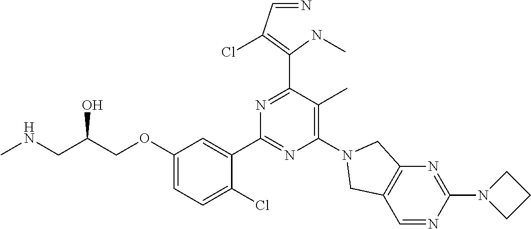 [SMILES: CNC[C@@H](O)COc1ccc(Cl)c(-c2nc(/C(NC)=C(\Cl)C=N)c(C)c(N3Cc4cnc(N5CCC5)nc4C3)n2)c1] | 0.002 | 0.01749 | 596 |
| 341 | 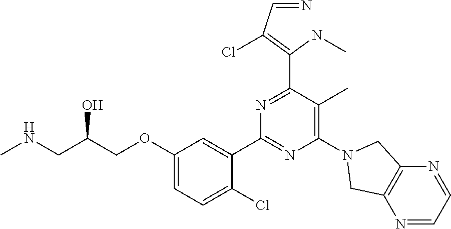 [SMILES: CNC[C@@H](O)COc1ccc(Cl)c(-c2nc(/C(NC)=C(\Cl)C=N)c(C)c(N3Cc4nccnc4C3)n2)c1] | 0.0027 | 0.18444 | 541 |
| 342 | 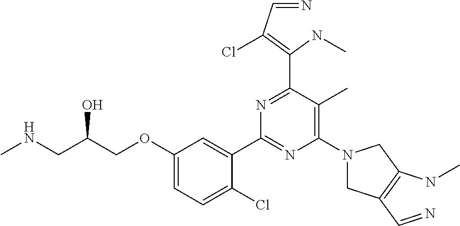 | 0.00274 | 0.12292 | 543.1 |
| 343 | 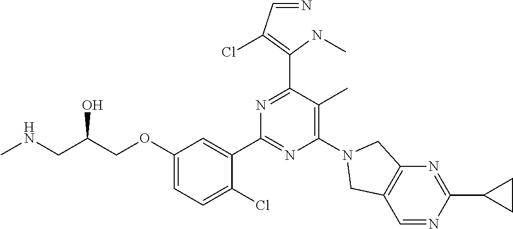 [SMILES: CNC[C@@H](O)COc1ccc(Cl)c(-c2nc(/C(NC)=C(\Cl)C=N)c(C)c(N3Cc4cnc(C5CC5)nc4C3)n2)c1] | 0.00176 | 0.05417 | 582.09 |
| 344 | 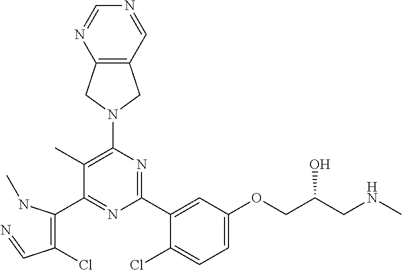 [SMILES: CNC[C@@H](O)COc1ccc(Cl)c(-c2nc(/C(NC)=C(\Cl)C=N)c(C)c(N3Cc4cncnc4C3)n2)c1] | 0.01285 | 0.71347 | 541.1 |
| 345 | 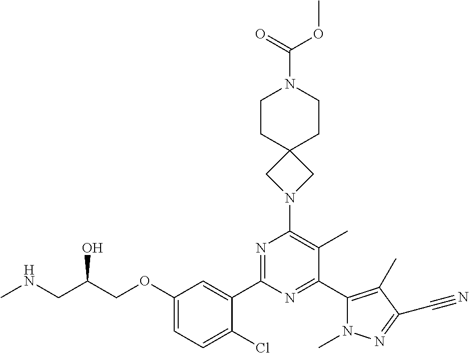 [SMILES: CNC[C@@H](O)COc1ccc(Cl)c(-c2nc(-c3c(C)c(C#N)nn3C)c(C)c(N3CC4(CCN(C(=O)OC)CC4)C3)n2)c1] | 0.00566 | 0.26177 | 609.3 |
| 346 | 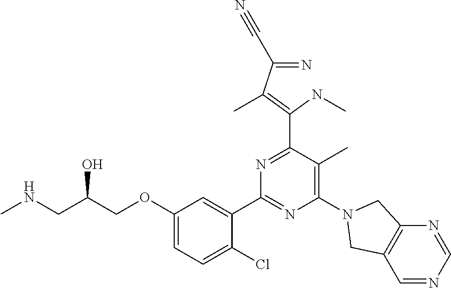 [SMILES: CNC[C@@H](O)COc1ccc(Cl)c(-c2nc(/C(NC)=C(\C)C(=N)C#N)c(C)c(N3Cc4cncnc4C3)n2)c1] | 0.00231 | 0.14038 | 546.2 |
| 347 | 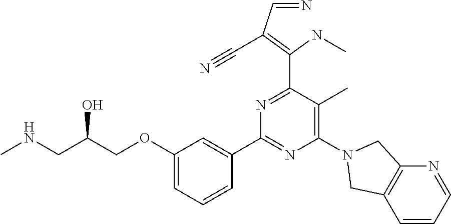 [SMILES: CNC[C@@H](O)COc1cccc(-c2nc(/C(NC)=C(\C#N)C=N)c(C)c(N3Cc4cccnc4C3)n2)c1] | 0.02822 | >2.0 | 497 |
| 348 | 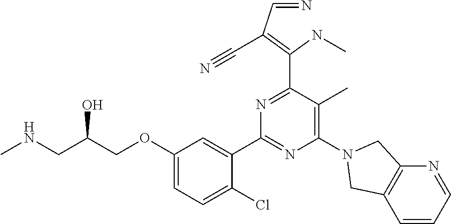 [SMILES: CNC[C@@H](O)COc1ccc(Cl)c(-c2nc(/C(NC)=C(\C#N)C=N)c(C)c(N3Cc4cccnc4C3)n2)c1] | 0.00567 | 1.67001 | 531 |
| 349 | 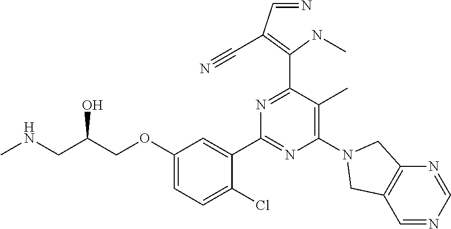 [SMILES: CNC[C@@H](O)COc1ccc(Cl)c(-c2nc(/C(NC)=C(\C#N)C=N)c(C)c(N3Cc4cncnc4C3)n2)c1] | 0.00781 | 3.11349 | 532 |
| 350 | 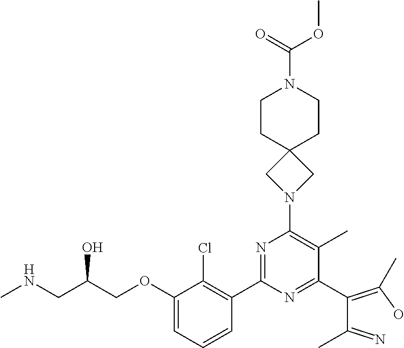 [SMILES: CNC[C@@H](O)COc1cccc(-c2nc(/C(C(C)=N)=C(\C)O)c(C)c(N3CC4(CCN(C(=O)OC)CC4)C3)n2)c1Cl] | 0.03715 | 7.19989 | 585.2 |

*Biochemical and Cellular-based (PABP1me2a) ICW (In Cell Western) assay results

In certain embodiments, the compound of Formula (I) is not a compound or pharmaceutically acceptable salt thereof as disclosed in PCT/US2014/028463, the disclosure of which is incorporated herein by reference.

In certain embodiments, compounds of Formula (I), wherein R^{A1} and R^{A2} are each —CH₃, i.e., to provide a Ring A of formula:

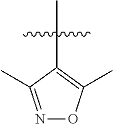

are specifically excluded.

In certain embodiments, compounds of Formula (I), wherein R^{2a}, R^{2b}, and R³ are any of the following specific combinations:
a. R^{2a} is hydrogen, R^{2b} is hydrogen, and R³ is —CH₃; and/or
b. R^{2a} is hydrogen, R^{2b} is hydrogen, and R³ is —F; and/or
c. R^{2a} is hydrogen, R^{2b} is hydrogen, and R³ is —Cl; and/or
d. R^{2a} is —Cl, R^{2b} is hydrogen, and R³ is —Cl; and/or
e. R^{2a} is —Cl, R^{2b} is hydrogen, and R³ is —F; and/or
f. R^{2a} is —F, R^{2b} is hydrogen, and R³ is —Cl; and/or
g. R^{2a} is —Cl, R^{2b} is hydrogen, and R³ is —CH₃; and/or
h. R^{2a} is —F, R^{2b} is hydrogen, and R³ is —CH₃; and/or
i. R^{2a} is —CF₃, R^{2b} is hydrogen, and R³ is —CH₃; and/or
j. R^{2a} is —CH₃, R^{2b} is hydrogen, and R³ is —CH₃; and/or
k. R^{2a} is hydrogen, R^{2b} is —Cl, and R³ is —CH₃;
   are specifically excluded.

In certain embodiments, any one or all of the below compounds, and pharmaceutically acceptable salts thereof, are specifically excluded:

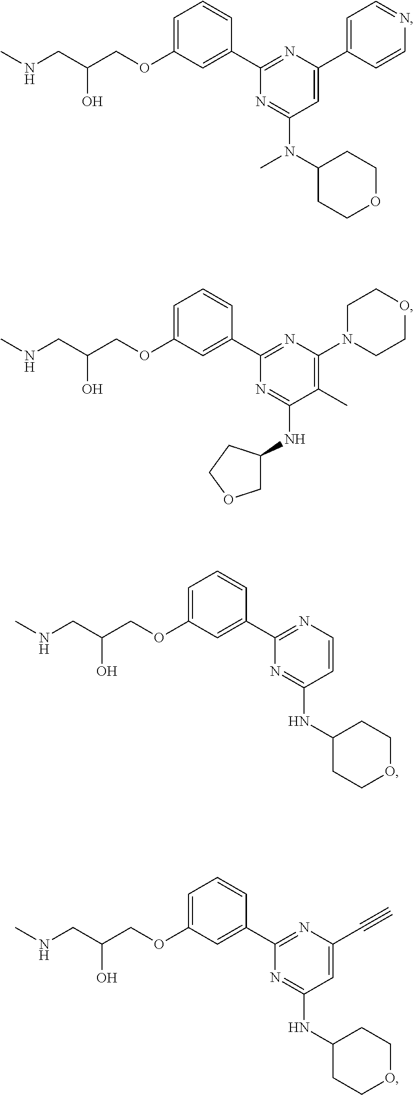 [SMILES: CNCC(O)COc1cccc(-c2nc(-c3ccncc3)cc(N(C)C3CCOCC3)n2)c1 ; CNCC(O)COc1cccc(-c2nc(N[C@@H]3CCOC3)c(C)c(N3CCOCC3)n2)c1 ; CNCC(O)COc1cccc(-c2nccc(NC3CCOCC3)n2)c1 ; C#Cc1cc(NC2CCOCC2)nc(-c2cccc(OCC(O)CNC)c2)n1]

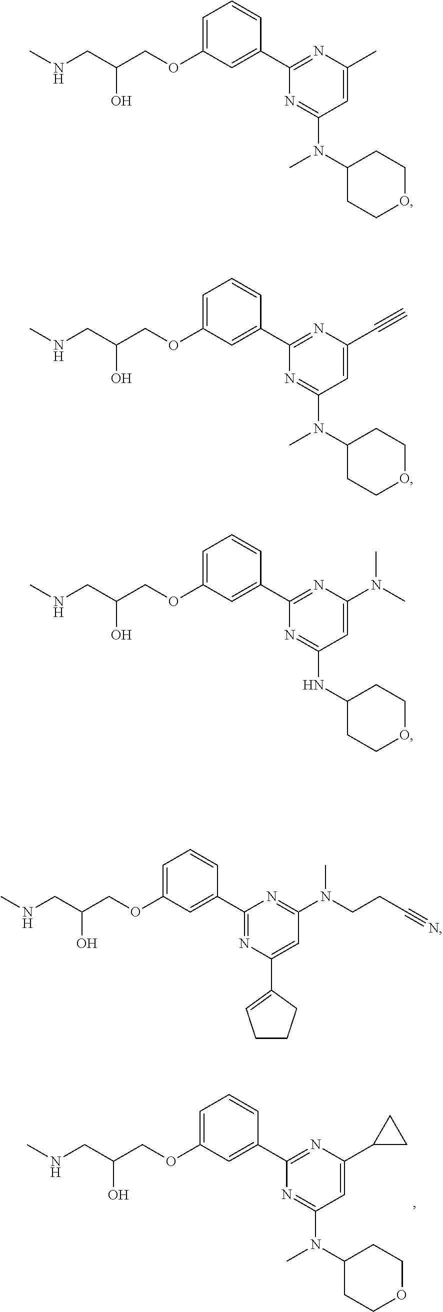

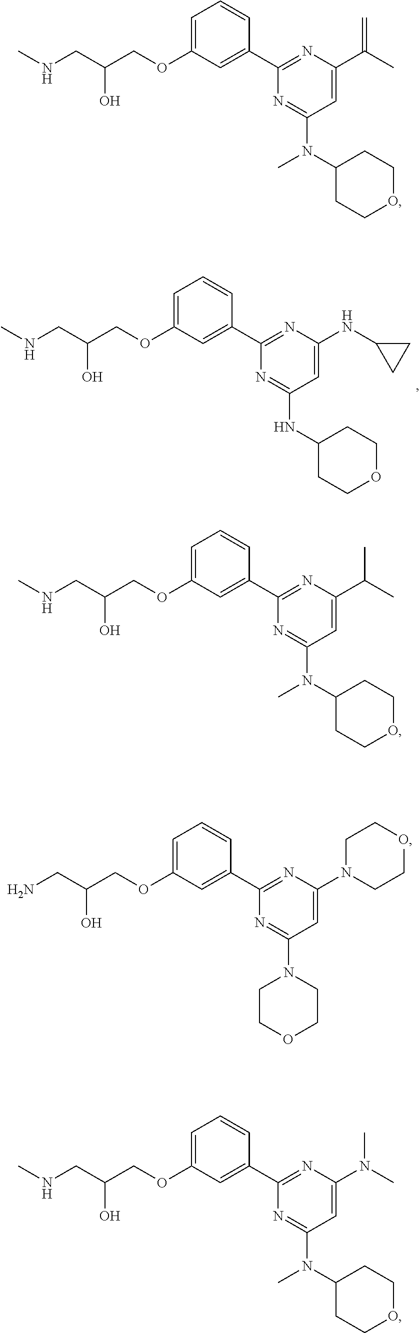

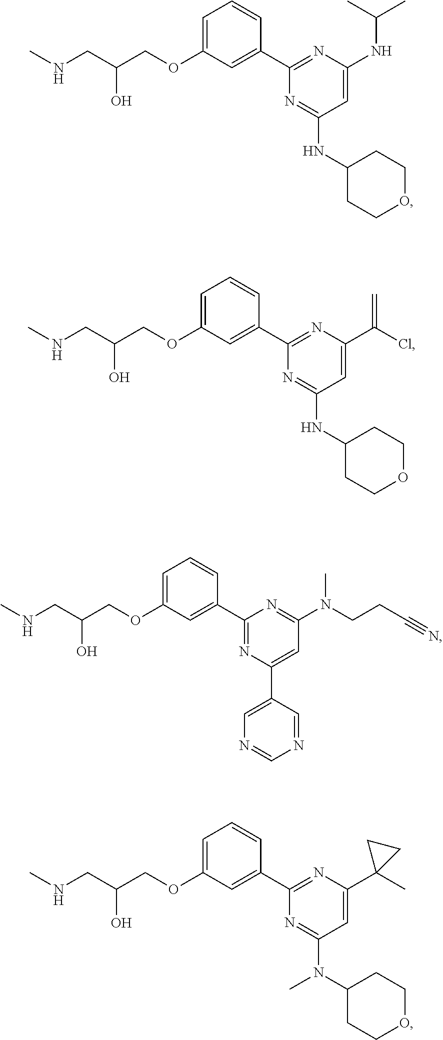

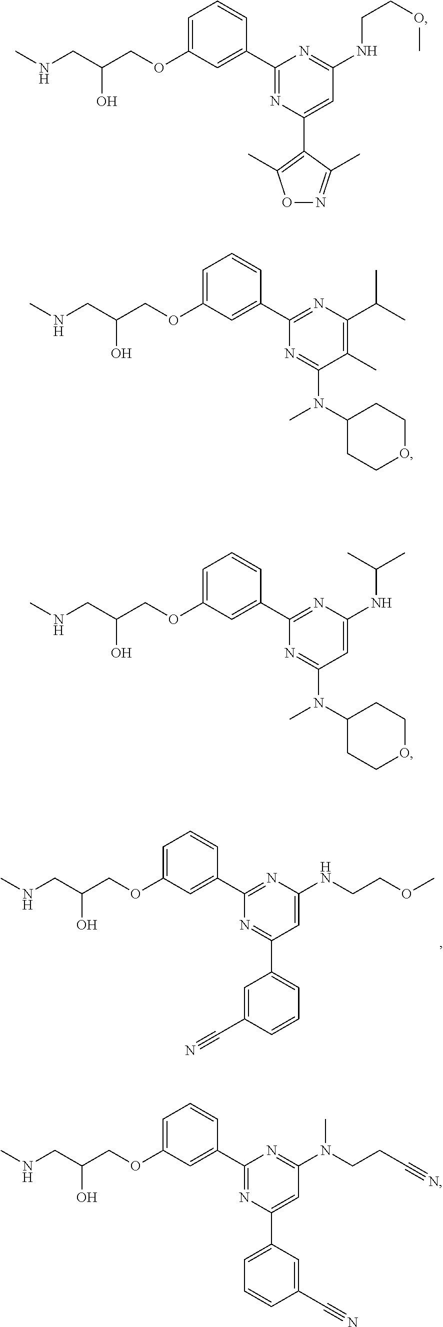

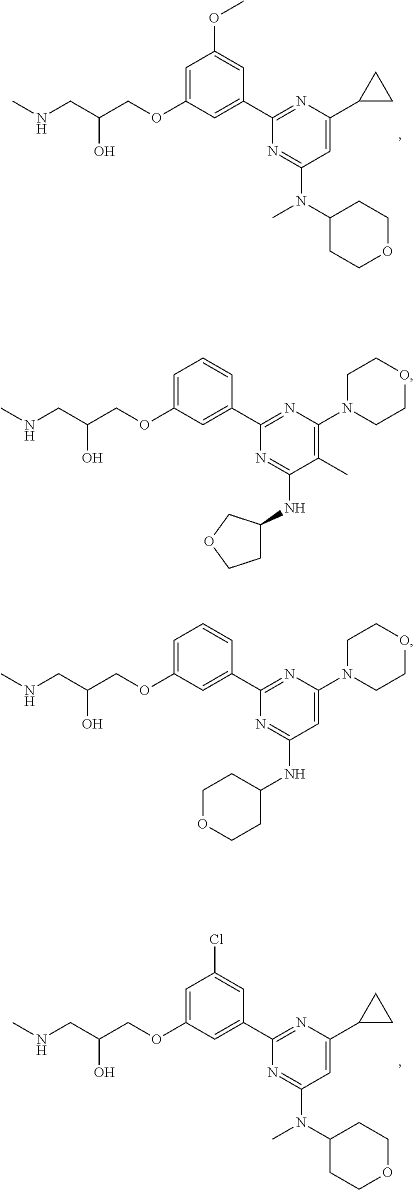 [SMILES: CNCC(O)COc1cc(OC)cc(-c2nc(C3CC3)cc(N(C)C3CCOCC3)n2)c1 ; CNCC(O)COc1cccc(-c2nc(N[C@H]3CCOC3)c(C)c(N3CCOCC3)n2)c1 ; CNCC(O)COc1cccc(-c2nc(NC3CCOCC3)cc(N3CCOCC3)n2)c1 ; CNCC(O)COc1cc(Cl)cc(-c2nc(C3CC3)cc(N(C)C3CCOCC3)n2)c1]

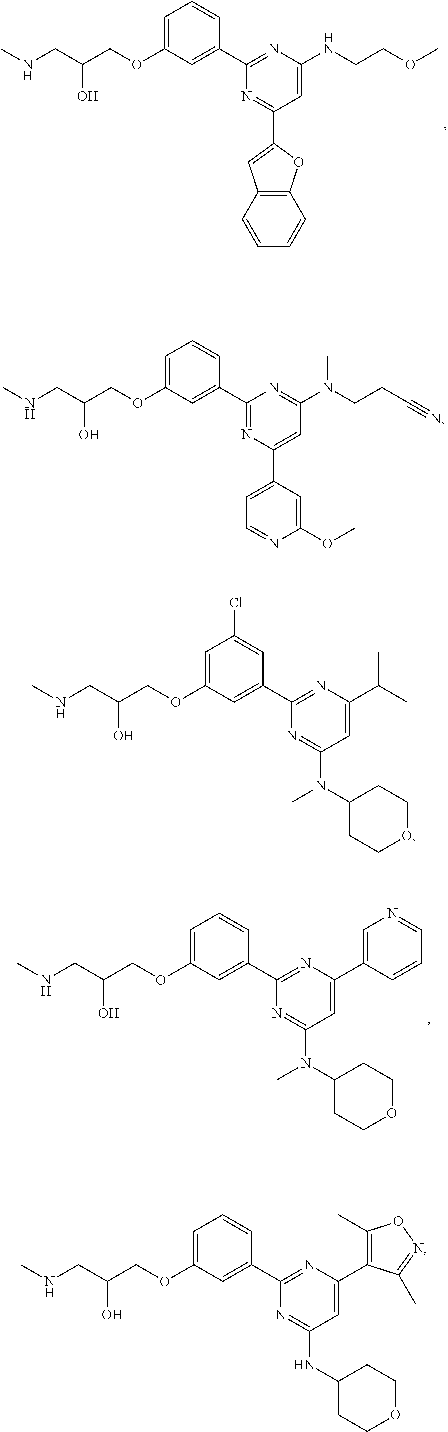

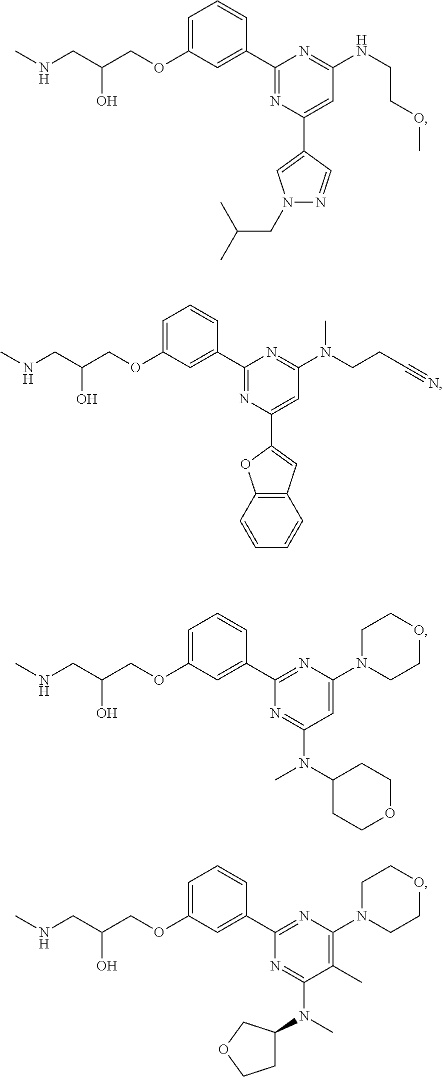

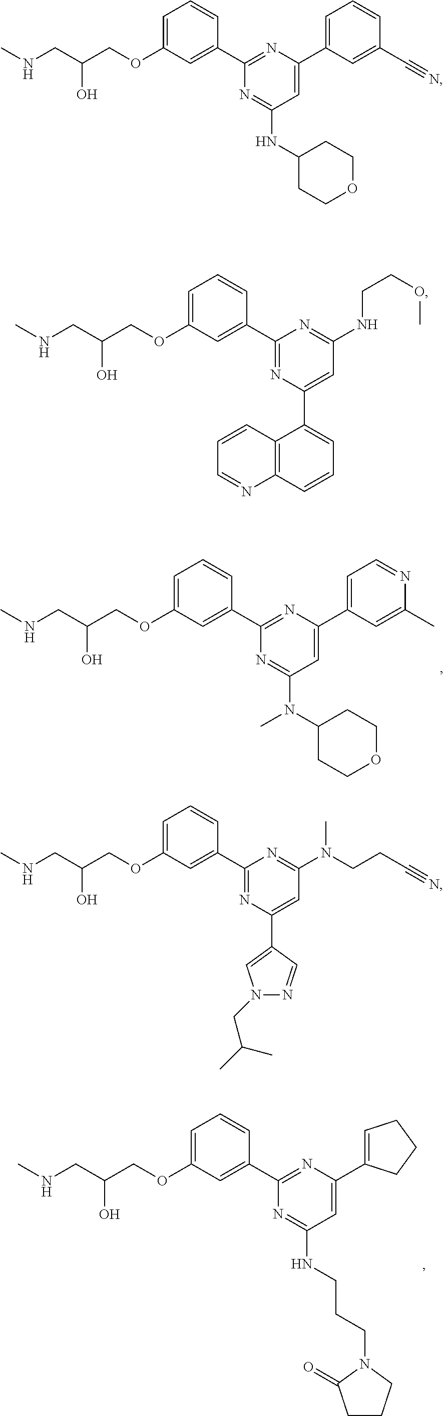

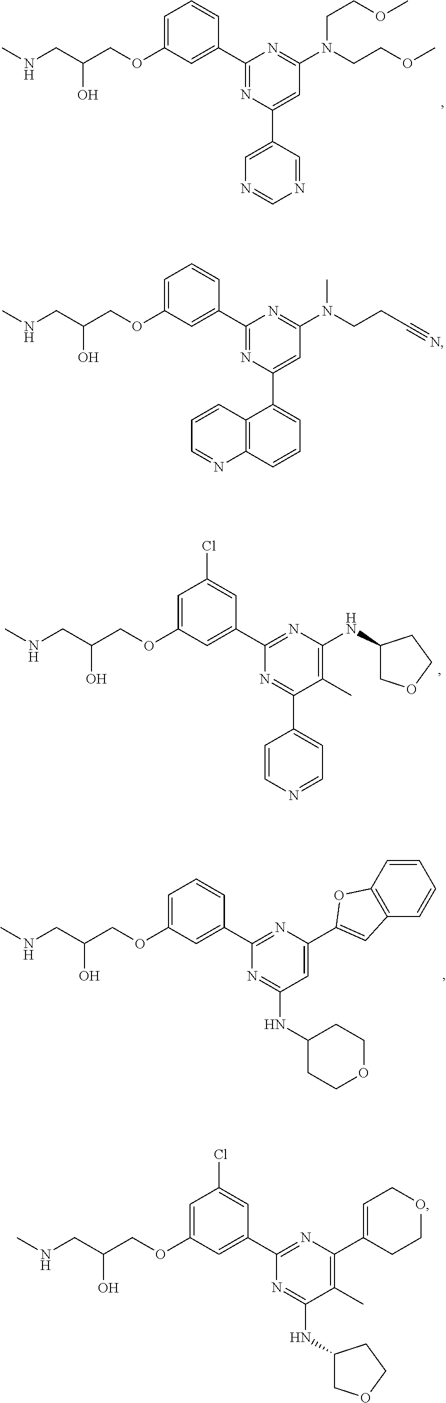

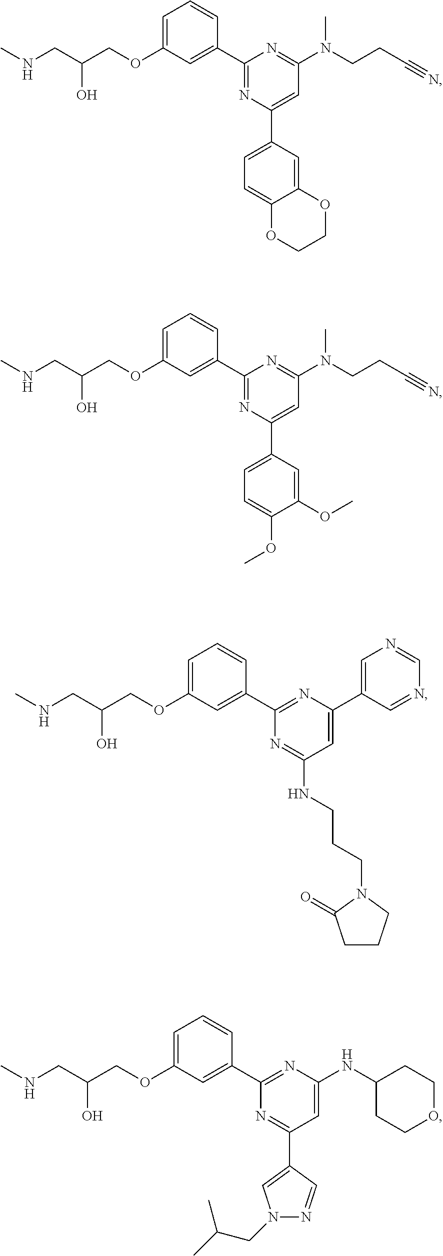 [SMILES: CNCC(O)COc1cccc(-c2nc(-c3ccc4c(c3)OCCO4)cc(N(C)CCC#N)n2)c1 ; CNCC(O)COc1cccc(-c2nc(-c3ccc(OC)c(OC)c3)cc(N(C)CCC#N)n2)c1 ; CNCC(O)COc1cccc(-c2nc(NCCCN3CCCC3=O)cc(-c3cncnc3)n2)c1 ; CNCC(O)COc1cccc(-c2nc(NC3CCOCC3)cc(-c3cnn(CC(C)C)c3)n2)c1]

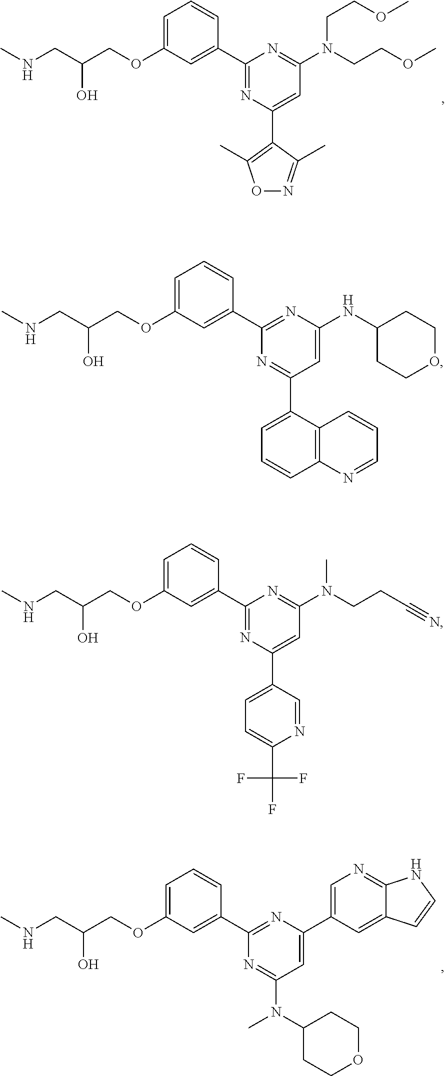

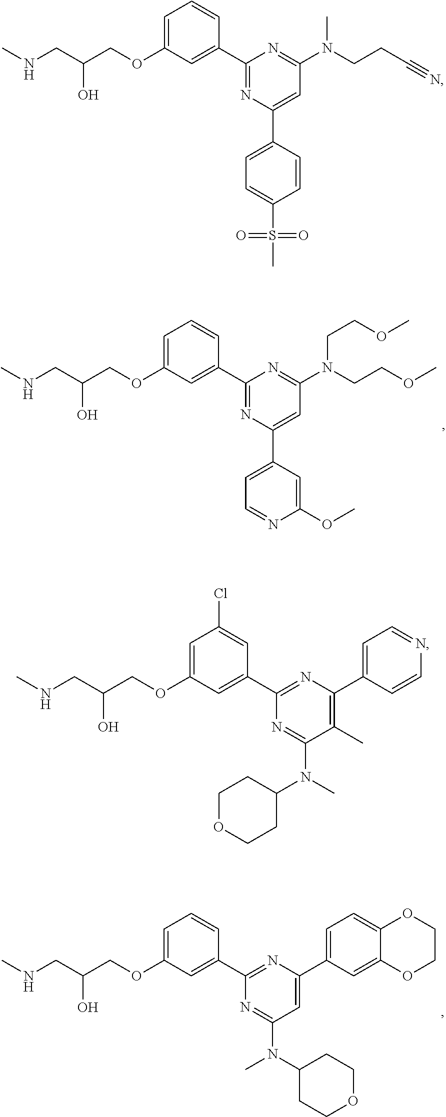

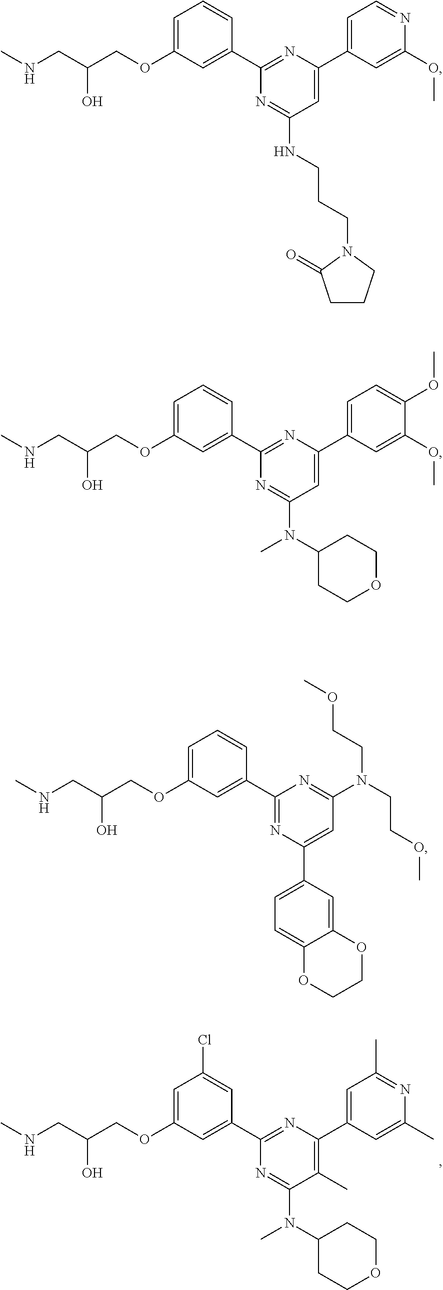

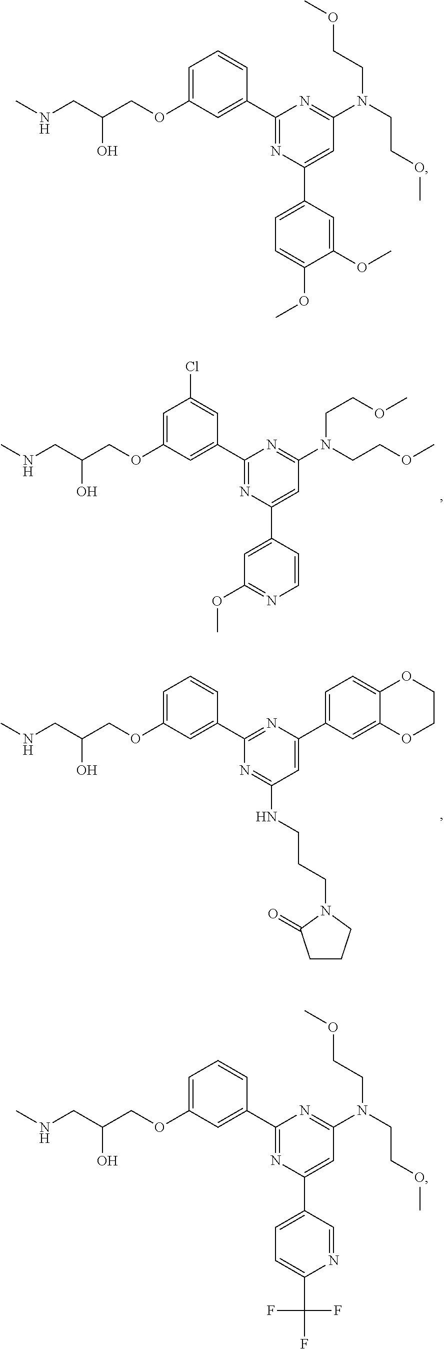

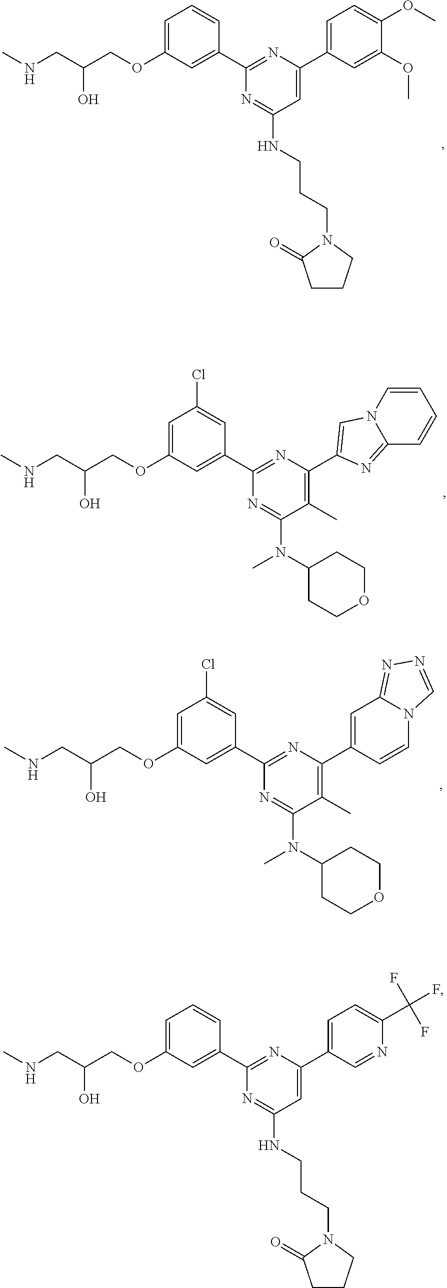

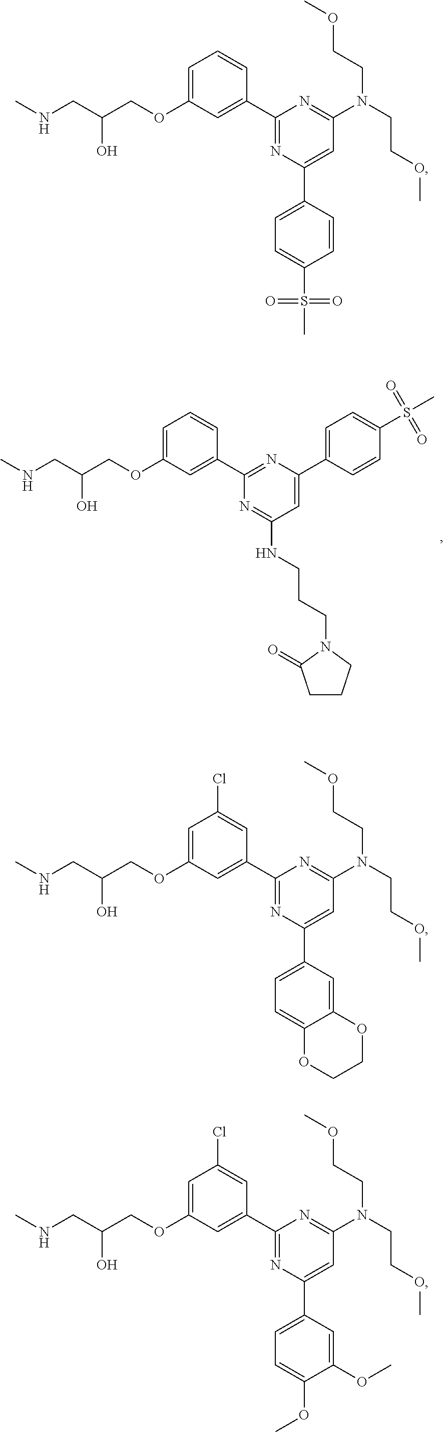

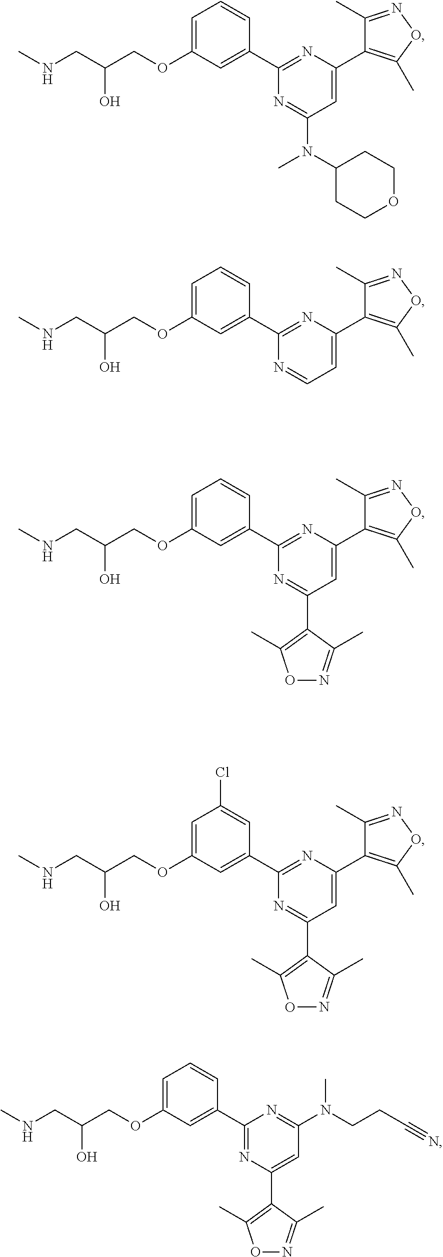 [SMILES: CNCC(O)COc1cccc(-c2nc(-c3c(C)noc3C)cc(N(C)C3CCOCC3)n2)c1 ; CNCC(O)COc1cccc(-c2nccc(-c3c(C)noc3C)n2)c1 ; CNCC(O)COc1cccc(-c2nc(-c3c(C)noc3C)cc(-c3c(C)noc3C)n2)c1 ; CNCC(O)COc1cc(Cl)cc(-c2nc(-c3c(C)noc3C)cc(-c3c(C)noc3C)n2)c1 ; CNCC(O)COc1cccc(-c2nc(-c3c(C)noc3C)cc(N(C)CCC#N)n2)c1]

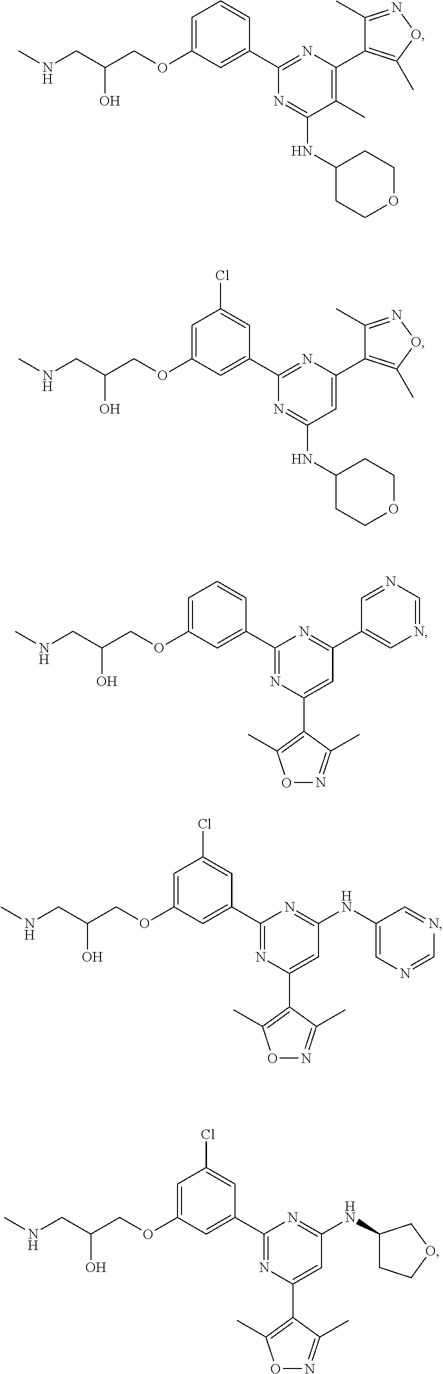 [SMILES: CNCC(O)COc1cccc(-c2nc(NC3CCOCC3)c(C)c(-c3c(C)noc3C)n2)c1 ; CNCC(O)COc1cc(Cl)cc(-c2nc(NC3CCOCC3)cc(-c3c(C)noc3C)n2)c1 ; CNCC(O)COc1cccc(-c2nc(-c3cncnc3)cc(-c3c(C)noc3C)n2)c1 ; CNCC(O)COc1cc(Cl)cc(-c2nc(Nc3cncnc3)cc(-c3c(C)noc3C)n2)c1 ; CNCC(O)COc1cc(Cl)cc(-c2nc(N[C@@H]3CCOC3)cc(-c3c(C)noc3C)n2)c1]

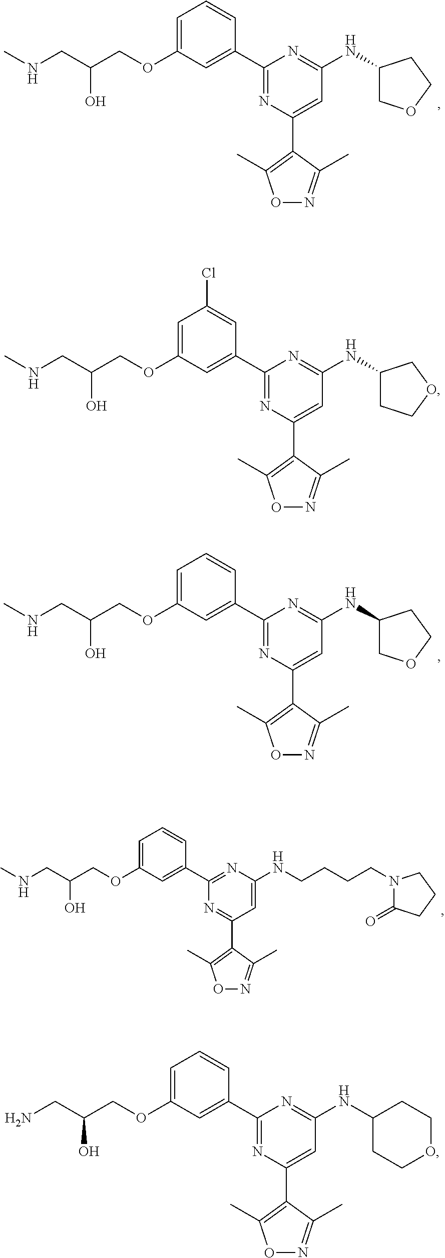 [SMILES: CNCC(O)COc1cccc(-c2nc(N[C@@H]3CCOC3)cc(-c3c(C)noc3C)n2)c1 ; CNCC(O)COc1cc(Cl)cc(-c2nc(N[C@H]3CCOC3)cc(-c3c(C)noc3C)n2)c1 ; CNCC(O)COc1cccc(-c2nc(N[C@H]3CCOC3)cc(-c3c(C)noc3C)n2)c1 ; CNCC(O)COc1cccc(-c2nc(NCCCCN3CCCC3=O)cc(-c3c(C)noc3C)n2)c1 ; Cc1noc(C)c1-c1cc(NC2CCOCC2)nc(-c2cccc(OC[C@@H](O)CN)c2)n1]

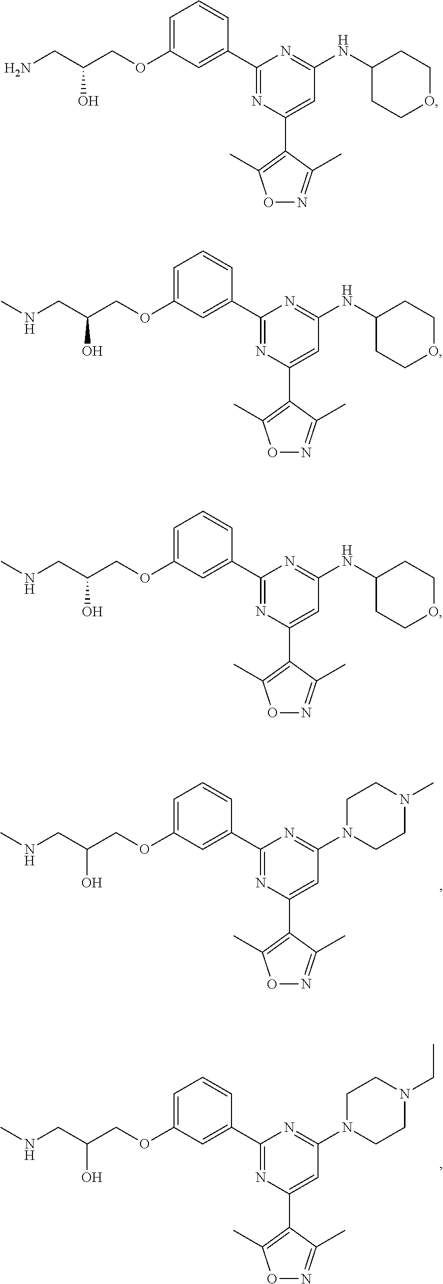

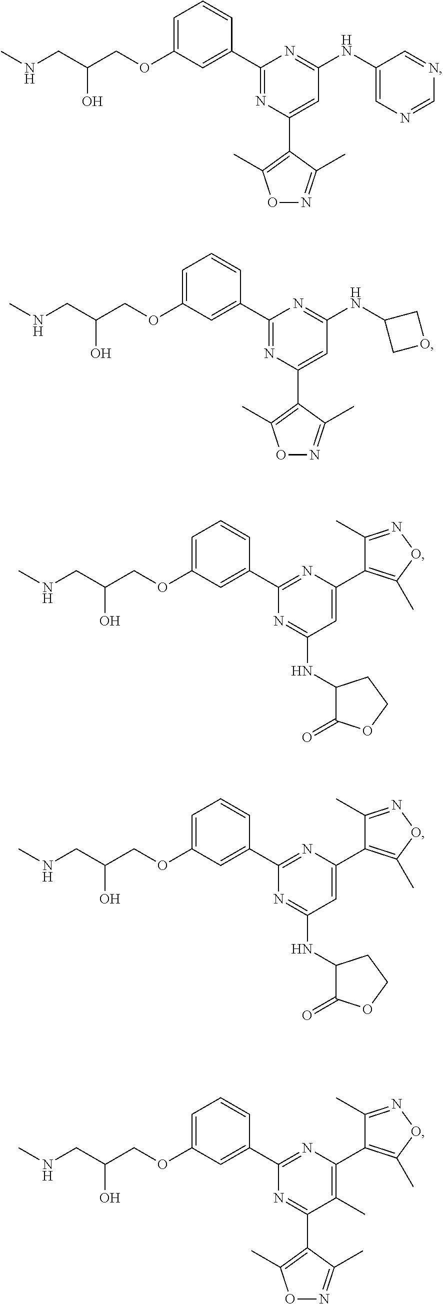 [SMILES: CNCC(O)COc1cccc(-c2nc(Nc3cncnc3)cc(-c3c(C)noc3C)n2)c1 ; CNCC(O)COc1cccc(-c2nc(NC3COC3)cc(-c3c(C)noc3C)n2)c1 ; CNCC(O)COc1cccc(-c2nc(NC3CCOC3=O)cc(-c3c(C)noc3C)n2)c1 ; CNCC(O)COc1cccc(-c2nc(NC3CCOC3=O)cc(-c3c(C)noc3C)n2)c1 ; CNCC(O)COc1cccc(-c2nc(-c3c(C)noc3C)c(C)c(-c3c(C)noc3C)n2)c1]

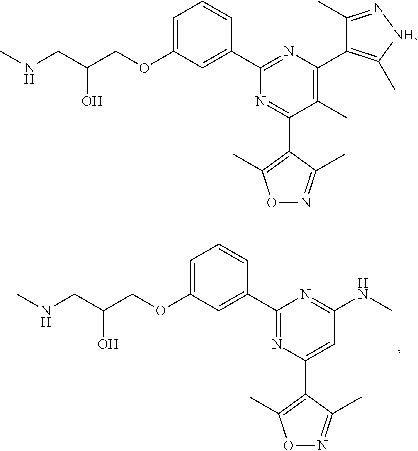

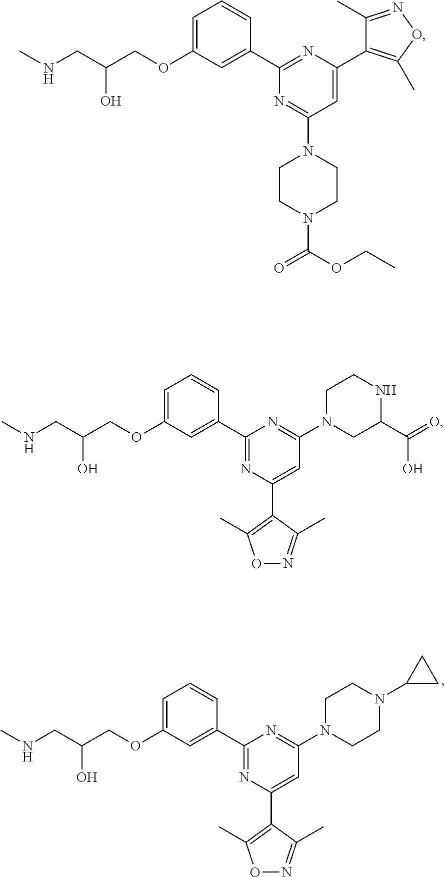 [SMILES: CCOC(=O)N1CCN(c2cc(-c3c(C)noc3C)nc(-c3cccc(OCC(O)CNC)c3)n2)CC1 ; CNCC(O)COc1cccc(-c2nc(-c3c(C)noc3C)cc(N3CCNC(C(=O)O)C3)n2)c1 ; CNCC(O)COc1cccc(-c2nc(-c3c(C)noc3C)cc(N3CCN(C4CC4)CC3)n2)c1]

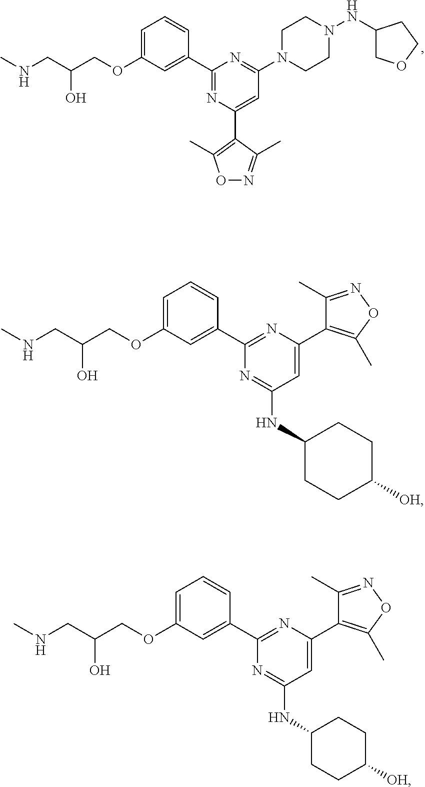 [SMILES: CNCC(O)COc1cccc(-c2nc(-c3c(C)noc3C)cc(N3CCN(NC4CCOC4)CC3)n2)c1 ; CNCC(O)COc1cccc(-c2nc(N[C@H]3CC[C@H](O)CC3)cc(-c3c(C)noc3C)n2)c1 ; CNCC(O)COc1cccc(-c2nc(N[C@H]3CC[C@@H](O)CC3)cc(-c3c(C)noc3C)n2)c1]

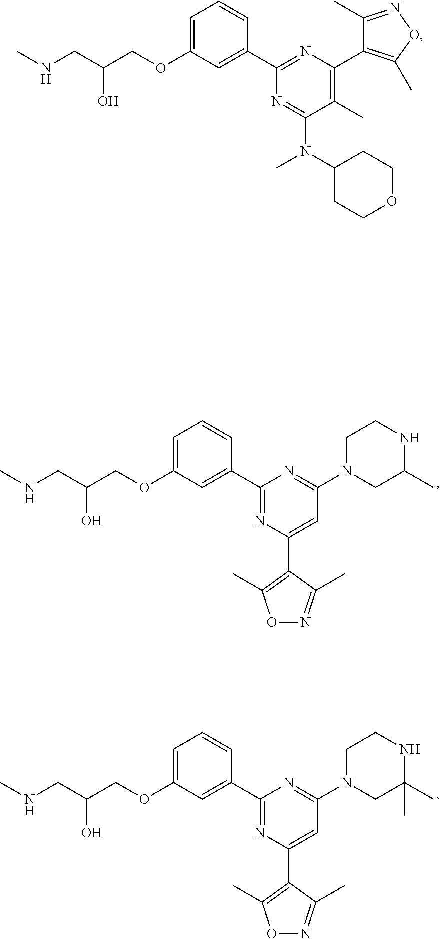

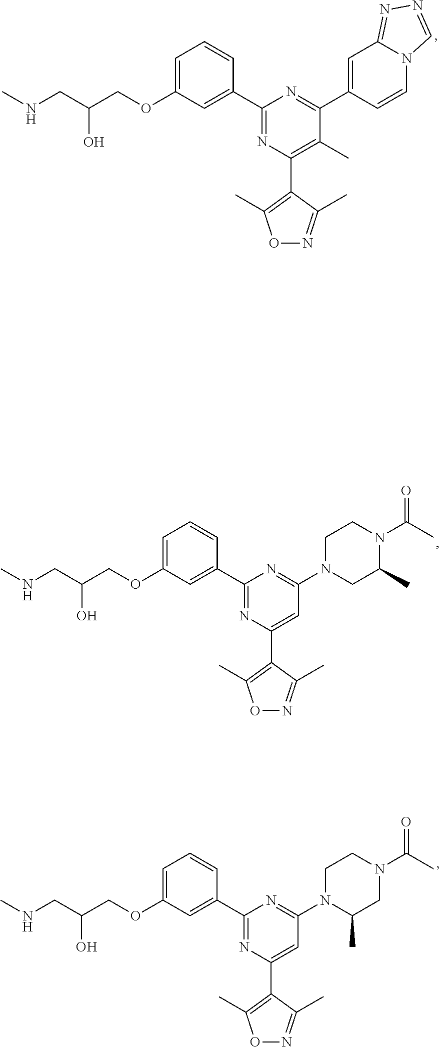

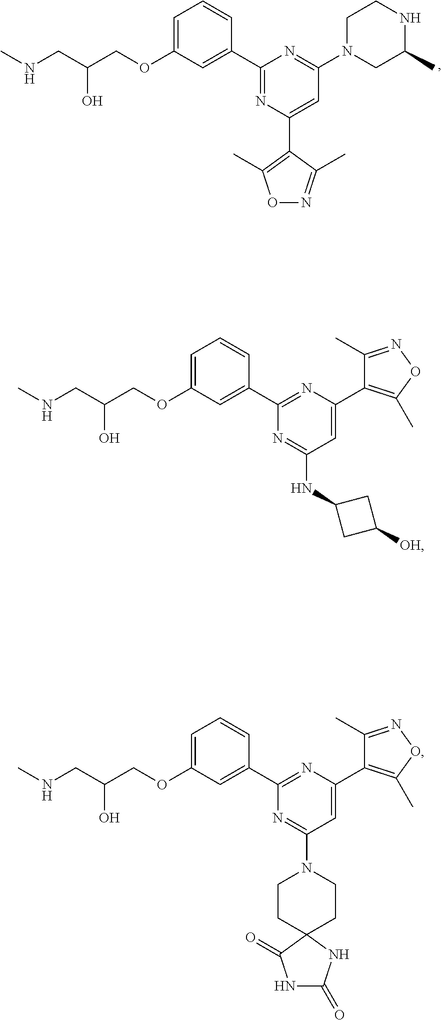

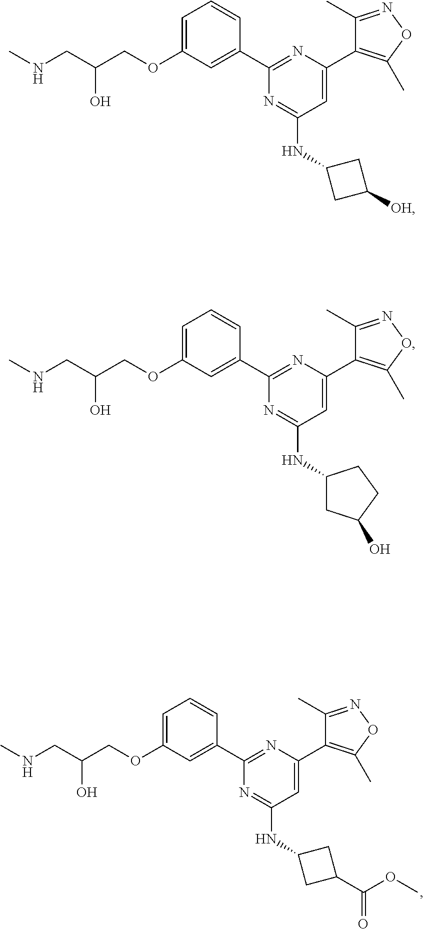

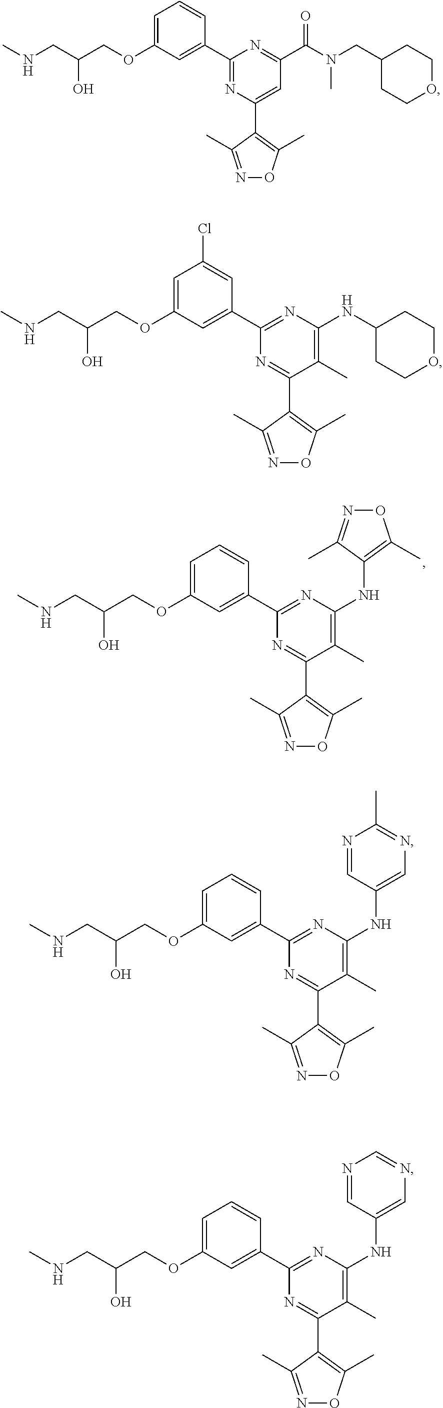

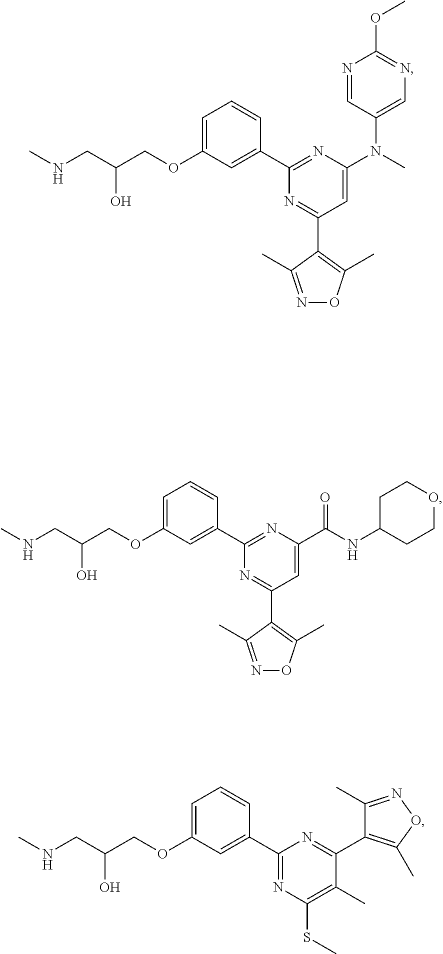

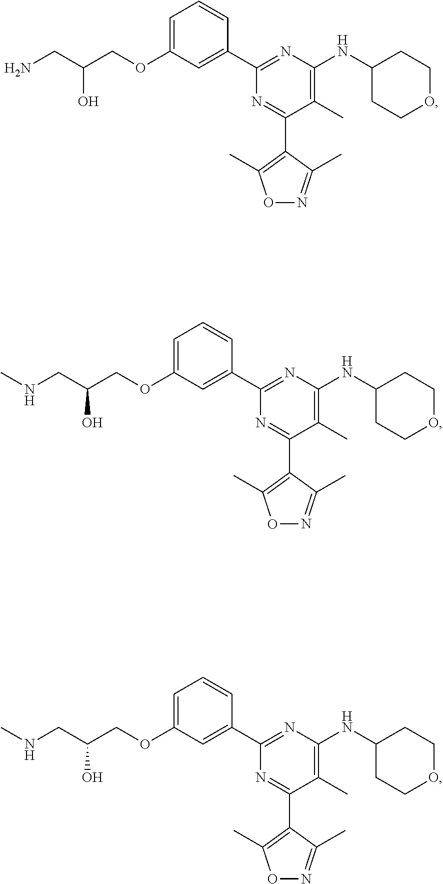 [SMILES: Cc1noc(C)c1-c1nc(-c2cccc(OCC(O)CN)c2)nc(NC2CCOCC2)c1C ; CNC[C@H](O)COc1cccc(-c2nc(NC3CCOCC3)c(C)c(-c3c(C)noc3C)n2)c1 ; CNC[C@@H](O)COc1cccc(-c2nc(NC3CCOCC3)c(C)c(-c3c(C)noc3C)n2)c1]

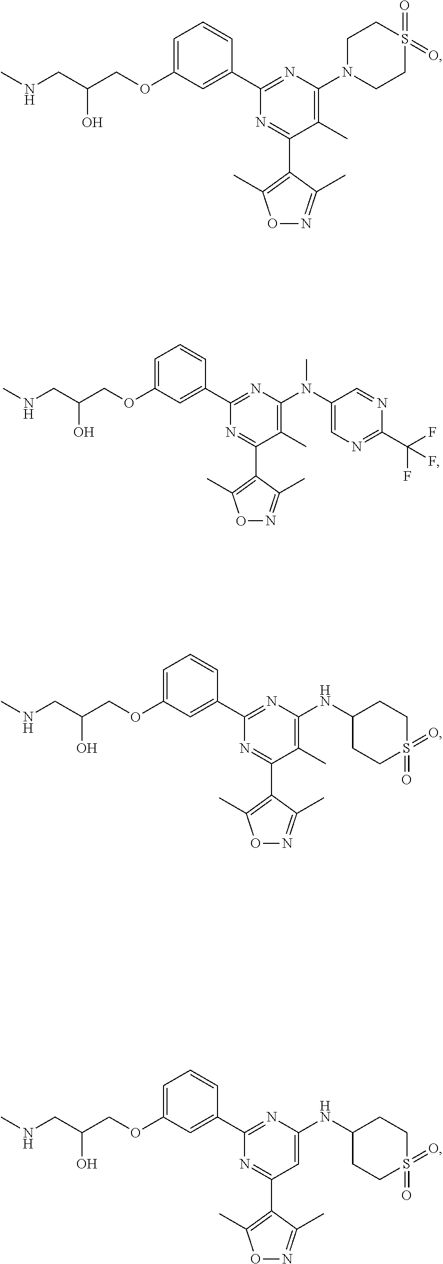 [SMILES: CNCC(O)COc1cccc(-c2nc(-c3c(C)noc3C)c(C)c(N3CCS(=O)(=O)CC3)n2)c1 ; CNCC(O)COc1cccc(-c2nc(-c3c(C)noc3C)c(C)c(N(C)c3cnc(C(F)(F)F)nc3)n2)c1 ; CNCC(O)COc1cccc(-c2nc(NC3CCS(=O)(=O)CC3)c(C)c(-c3c(C)noc3C)n2)c1 ; CNCC(O)COc1cccc(-c2nc(NC3CCS(=O)(=O)CC3)cc(-c3c(C)noc3C)n2)c1]

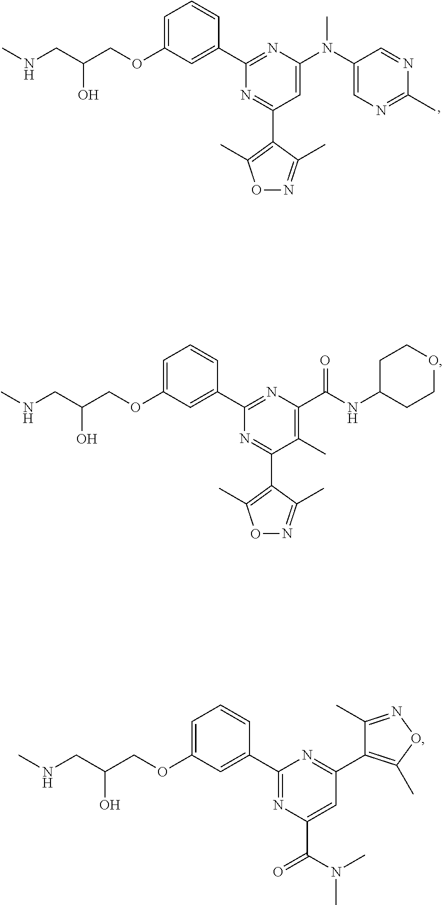

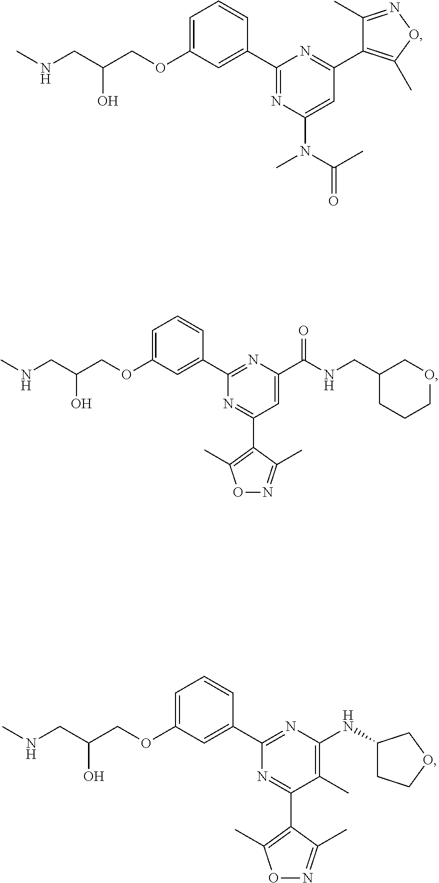 [SMILES: CNCC(O)COc1cccc(-c2nc(-c3c(C)noc3C)cc(N(C)C(C)=O)n2)c1 ; CNCC(O)COc1cccc(-c2nc(C(=O)NCC3CCCOC3)cc(-c3c(C)noc3C)n2)c1 ; CNCC(O)COc1cccc(-c2nc(N[C@H]3CCOC3)c(C)c(-c3c(C)noc3C)n2)c1]

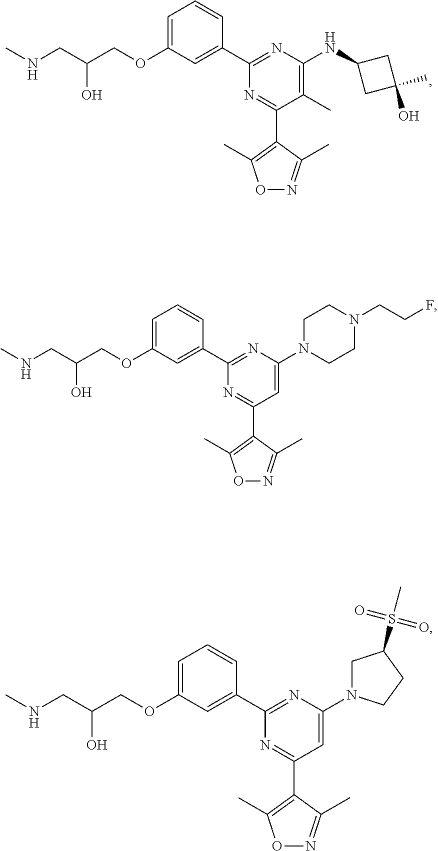

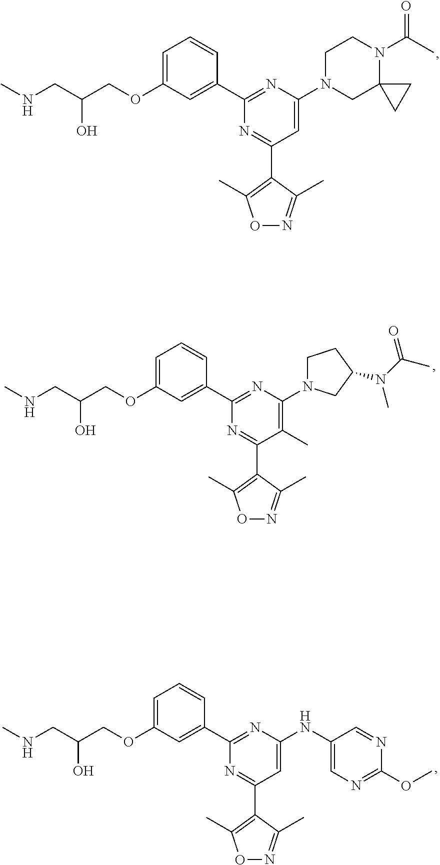

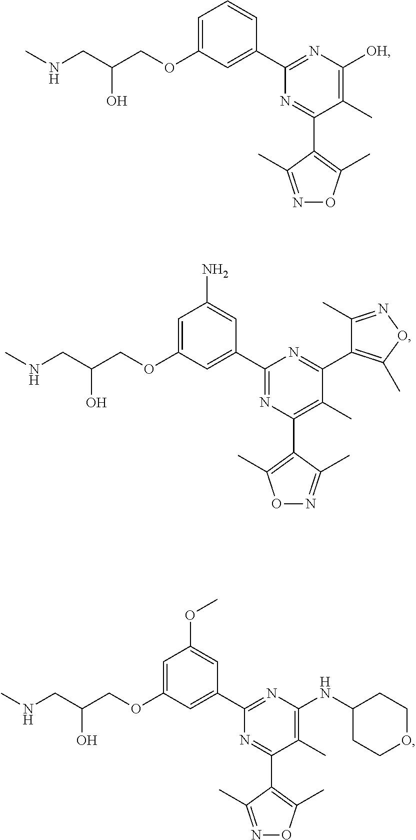

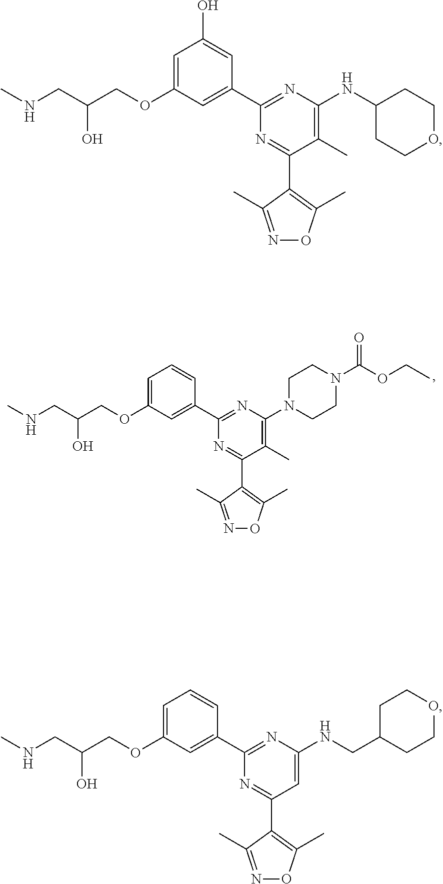

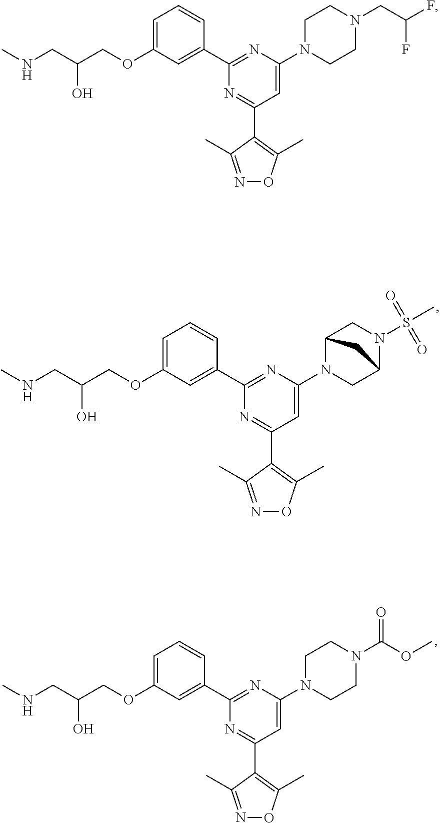

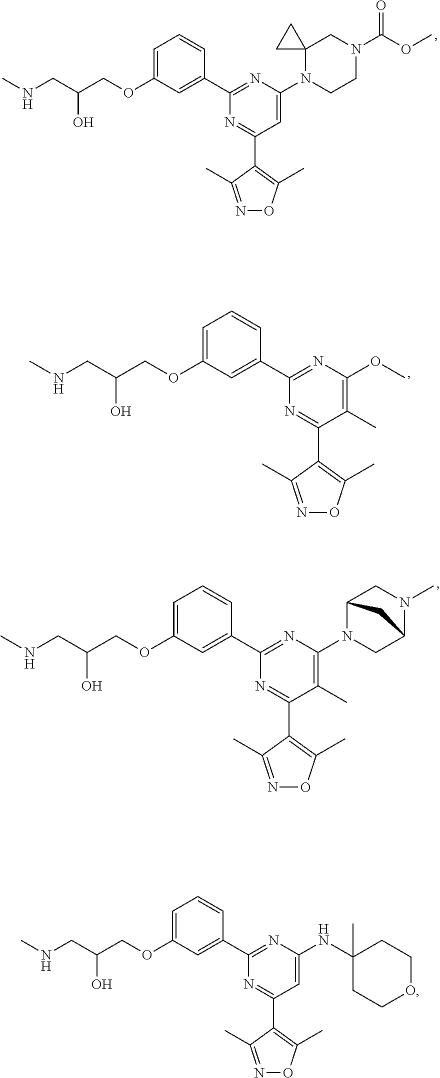

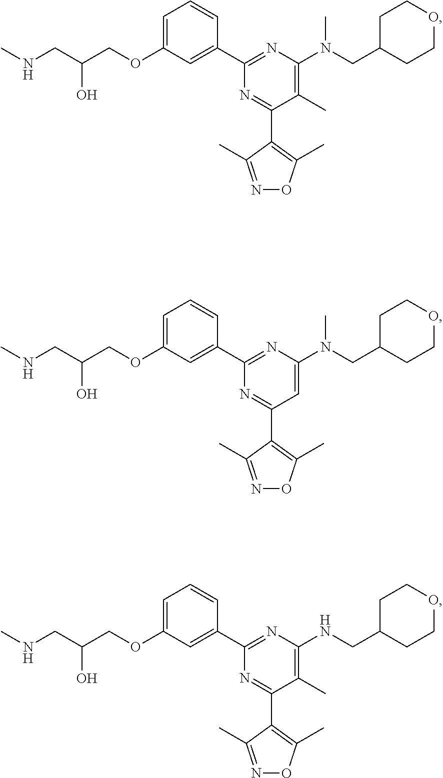 [SMILES: CNCC(O)COc1cccc(-c2nc(-c3c(C)noc3C)c(C)c(N(C)CC3CCOCC3)n2)c1 ; CNCC(O)COc1cccc(-c2nc(-c3c(C)noc3C)cc(N(C)CC3CCOCC3)n2)c1 ; CNCC(O)COc1cccc(-c2nc(NCC3CCOCC3)c(C)c(-c3c(C)noc3C)n2)c1]

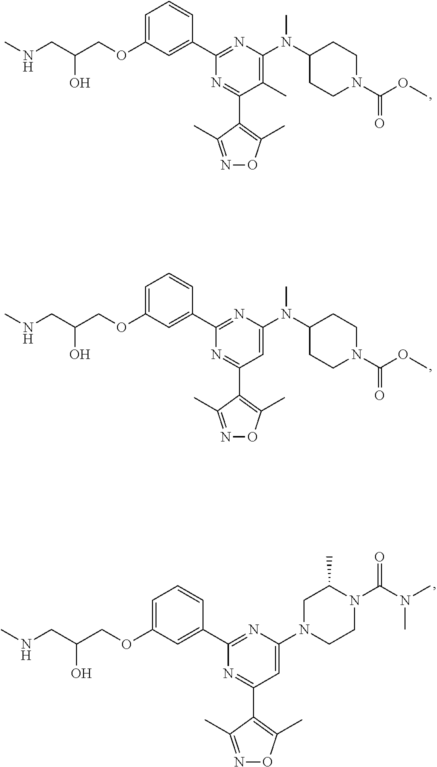

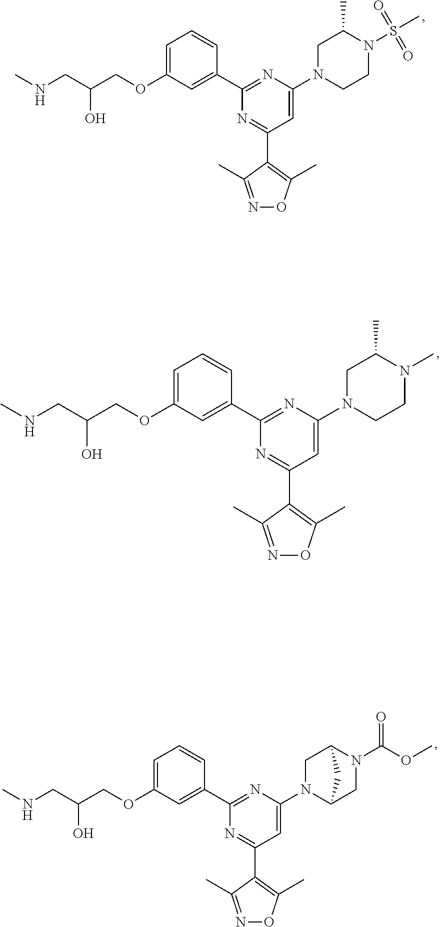

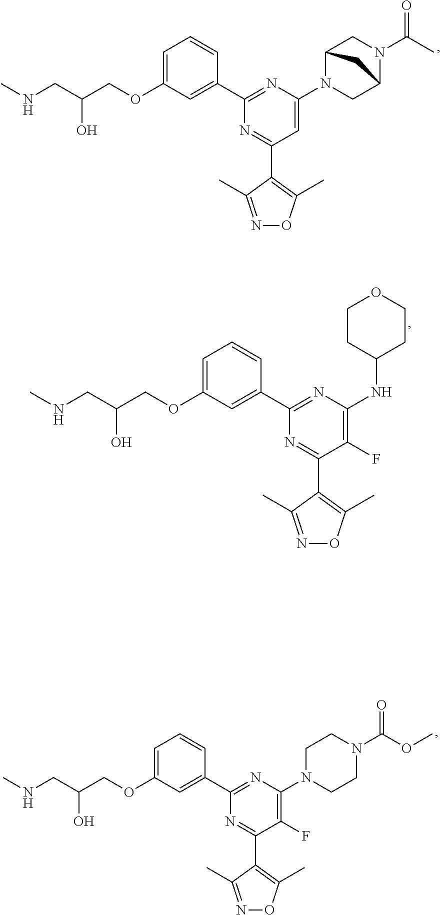

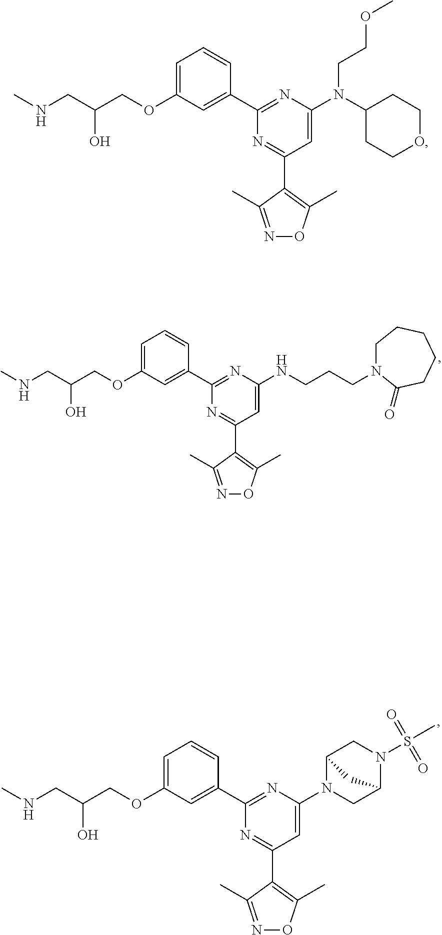

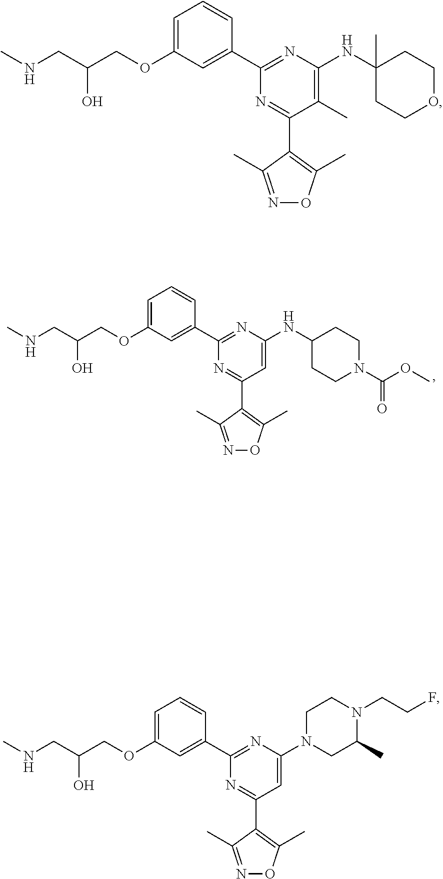

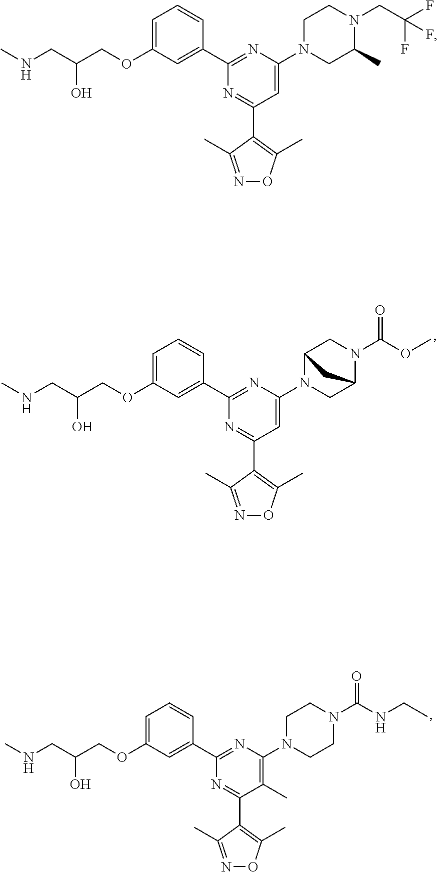

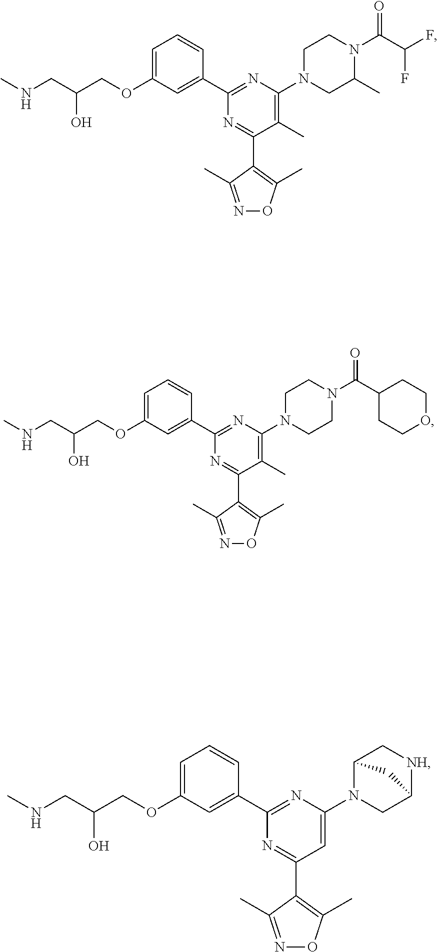 [SMILES: CNCC(O)COc1cccc(-c2nc(-c3c(C)noc3C)c(C)c(N3CCN(C(=O)C(F)F)C(C)C3)n2)c1 ; CNCC(O)COc1cccc(-c2nc(-c3c(C)noc3C)c(C)c(N3CCN(C(=O)C4CCOCC4)CC3)n2)c1 ; CNCC(O)COc1cccc(-c2nc(-c3c(C)noc3C)cc(N3C[C@H]4C[C@@H]3CN4)n2)c1]

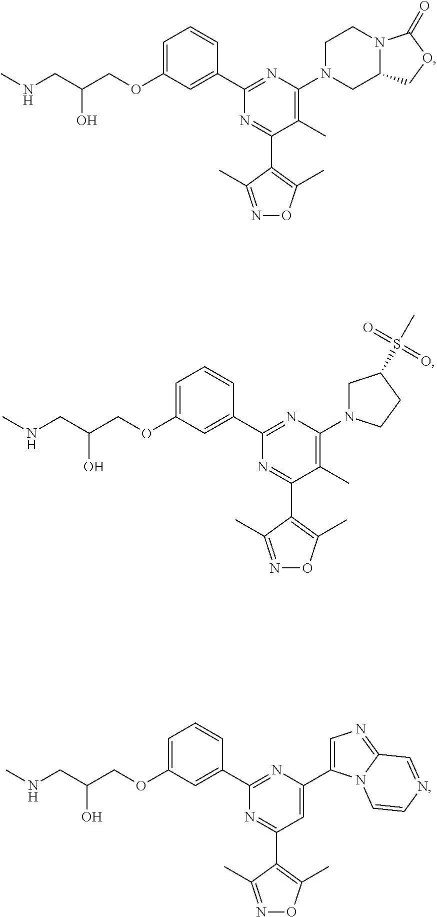 [SMILES: CNCC(O)COc1cccc(-c2nc(-c3c(C)noc3C)c(C)c(N3CCN4C(=O)OC[C@@H]4C3)n2)c1 ; CNCC(O)COc1cccc(-c2nc(-c3c(C)noc3C)c(C)c(N3CC[C@@H](S(C)(=O)=O)C3)n2)c1 ; CNCC(O)COc1cccc(-c2nc(-c3c(C)noc3C)cc(-c3cnc4cnccn34)n2)c1]

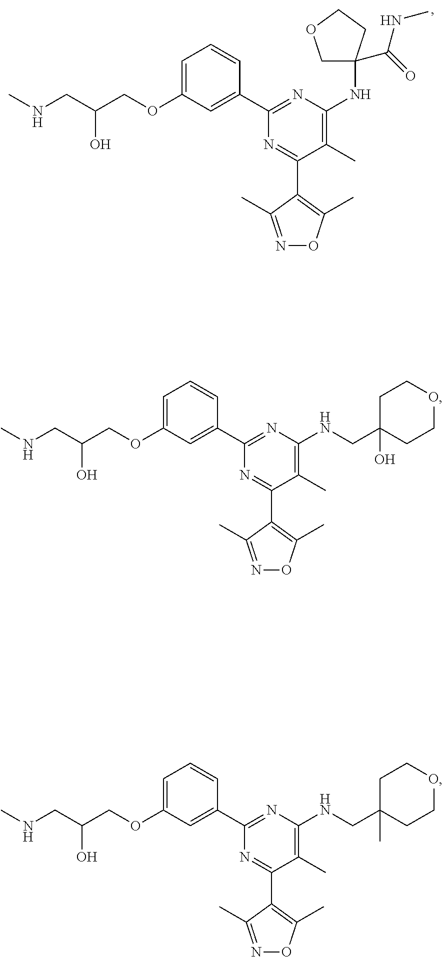

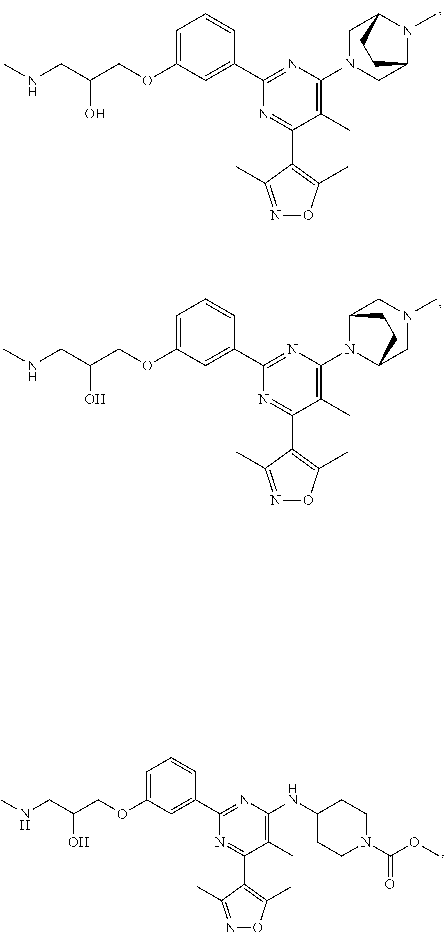

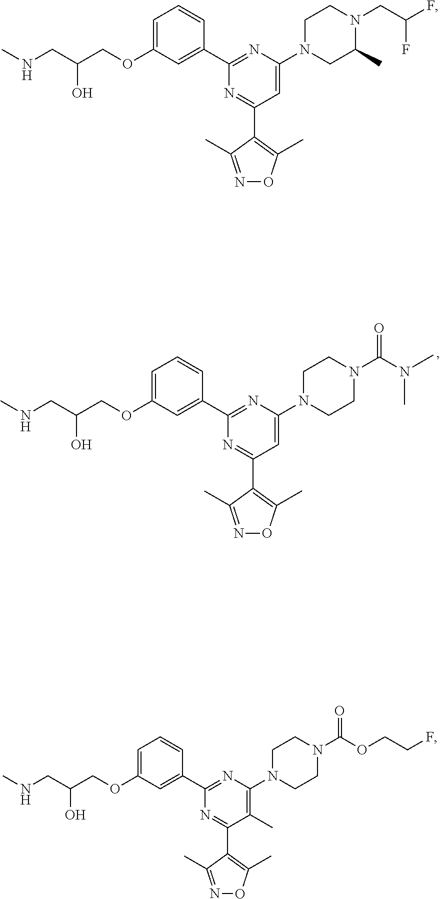

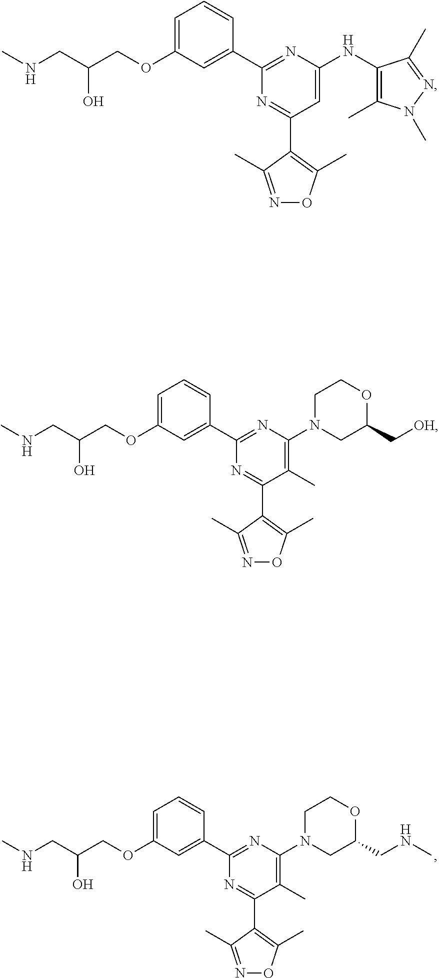

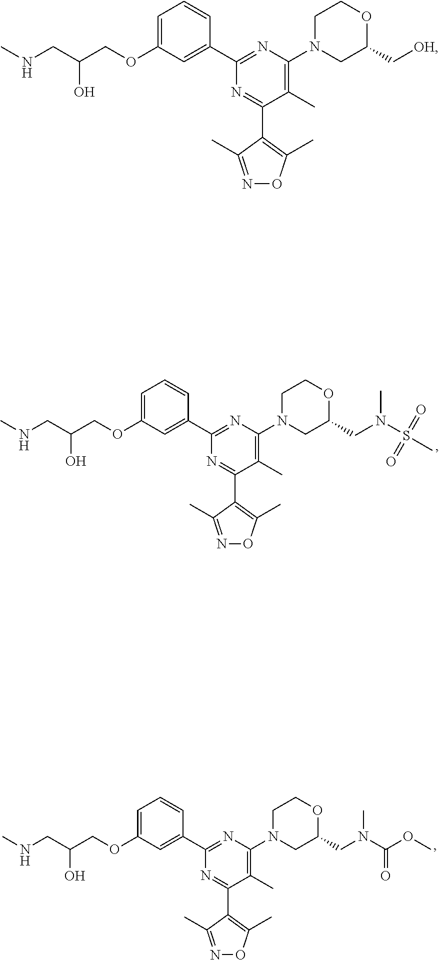

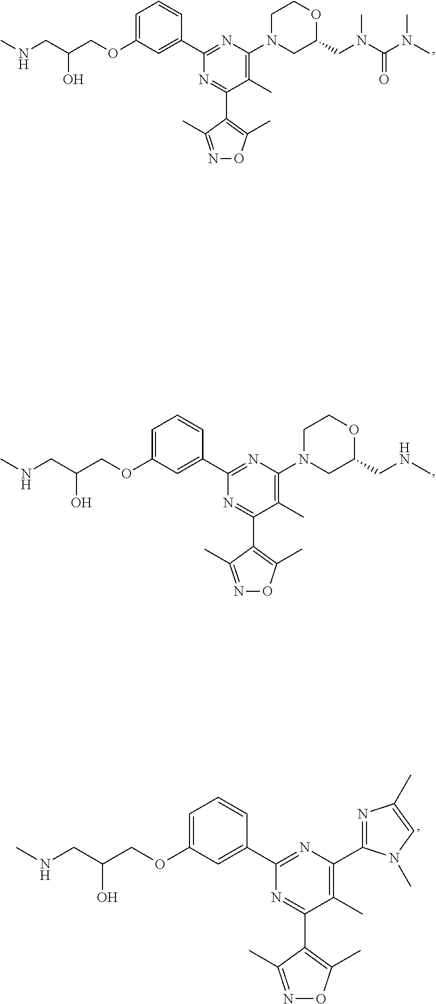

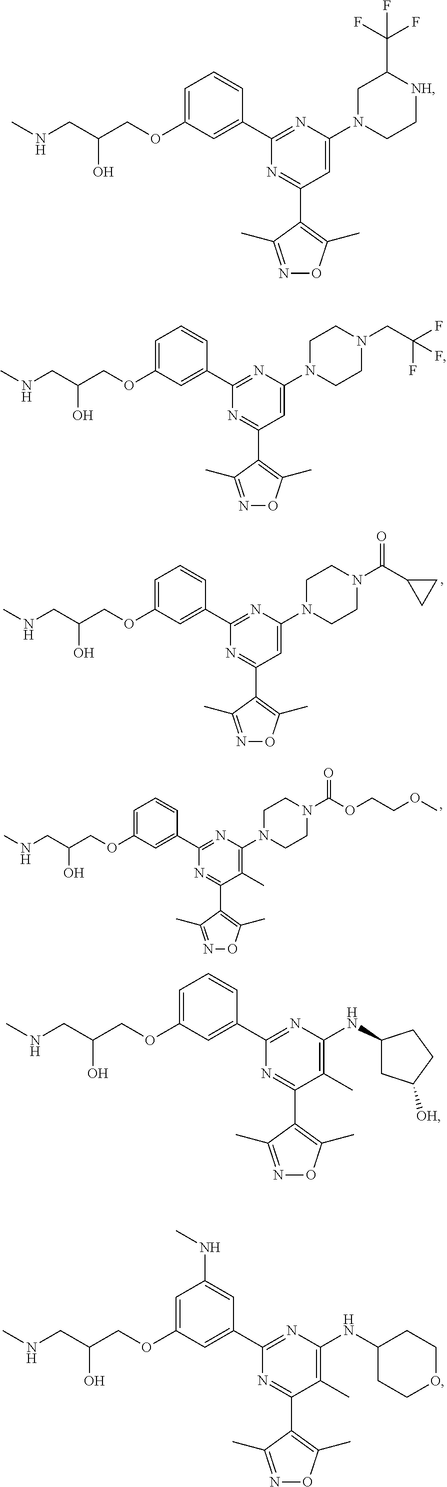

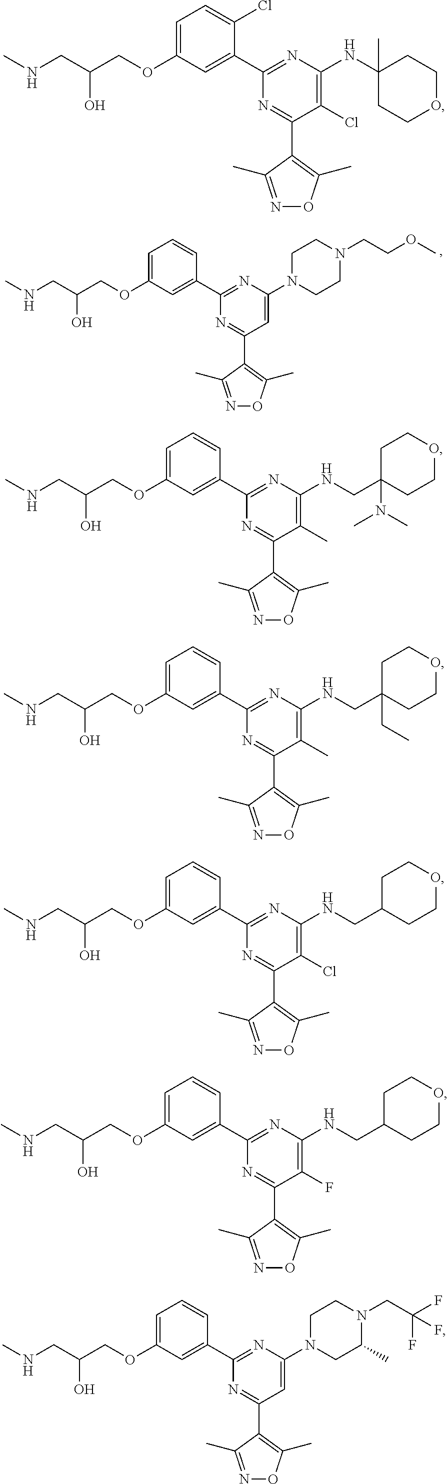

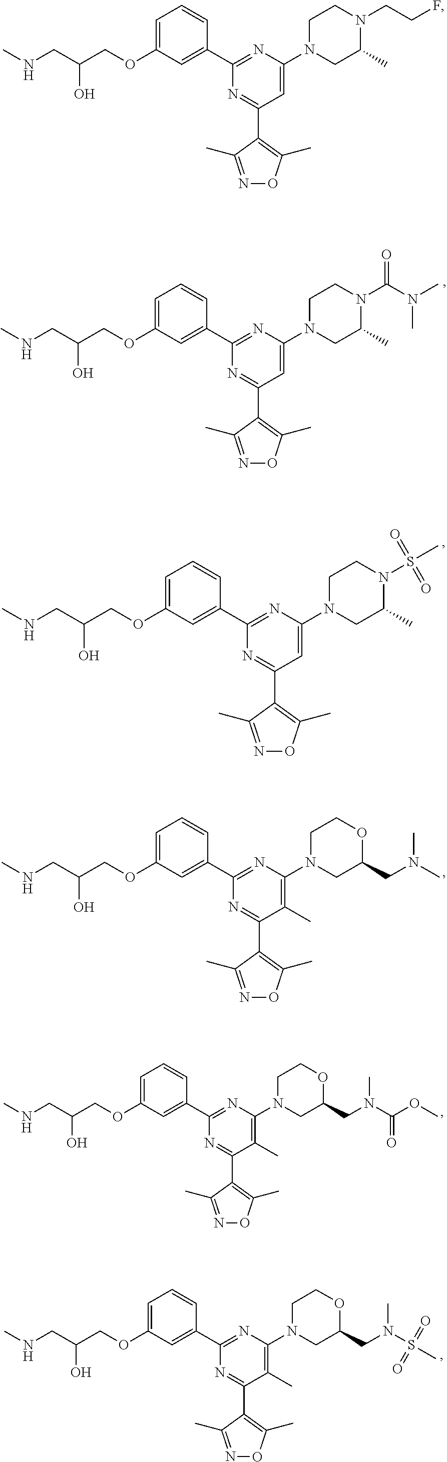

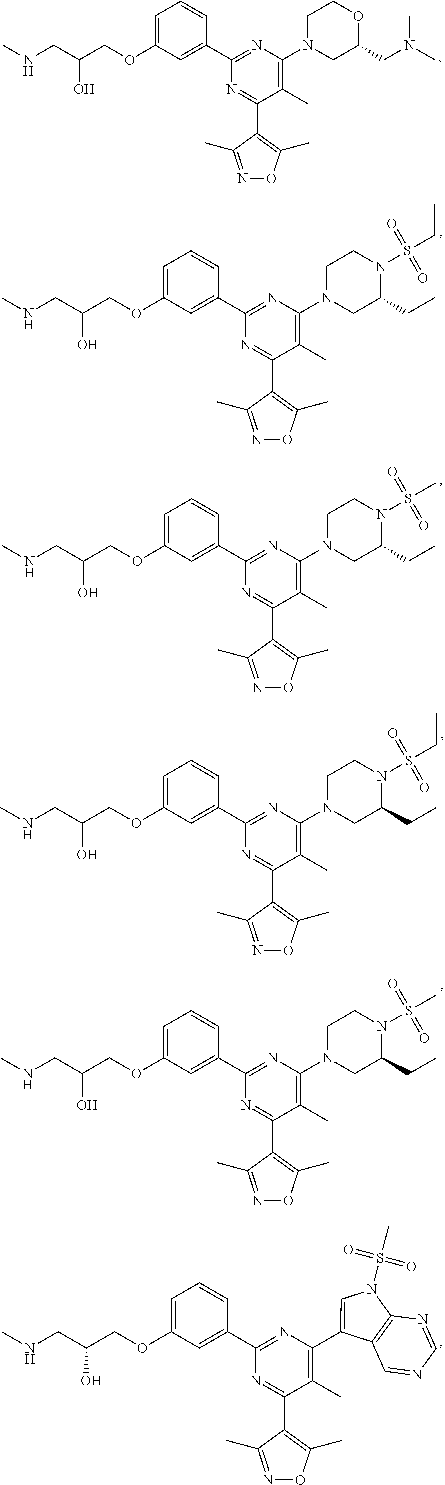

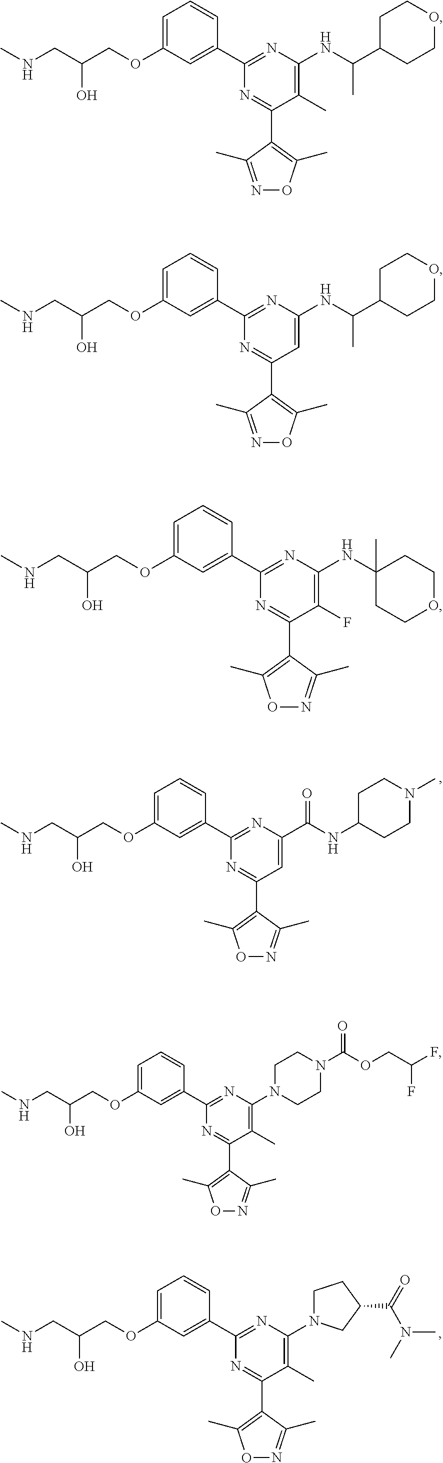

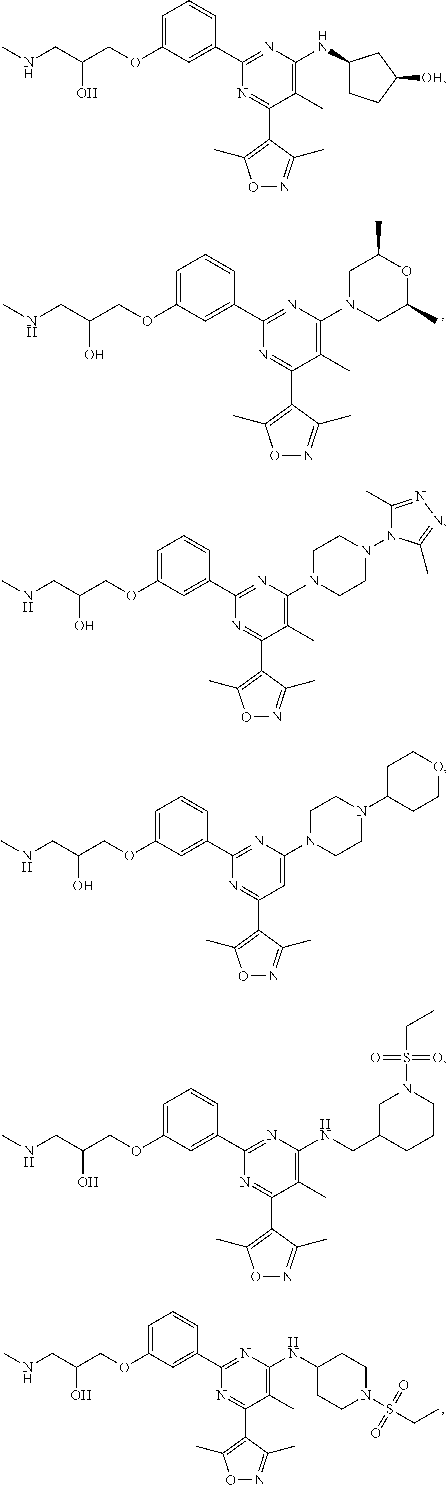

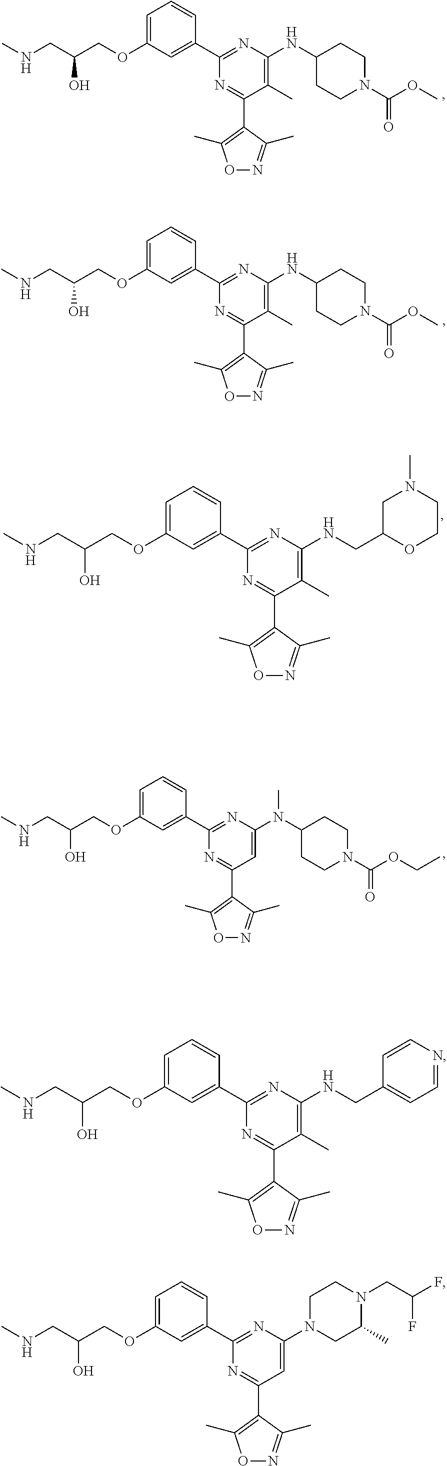

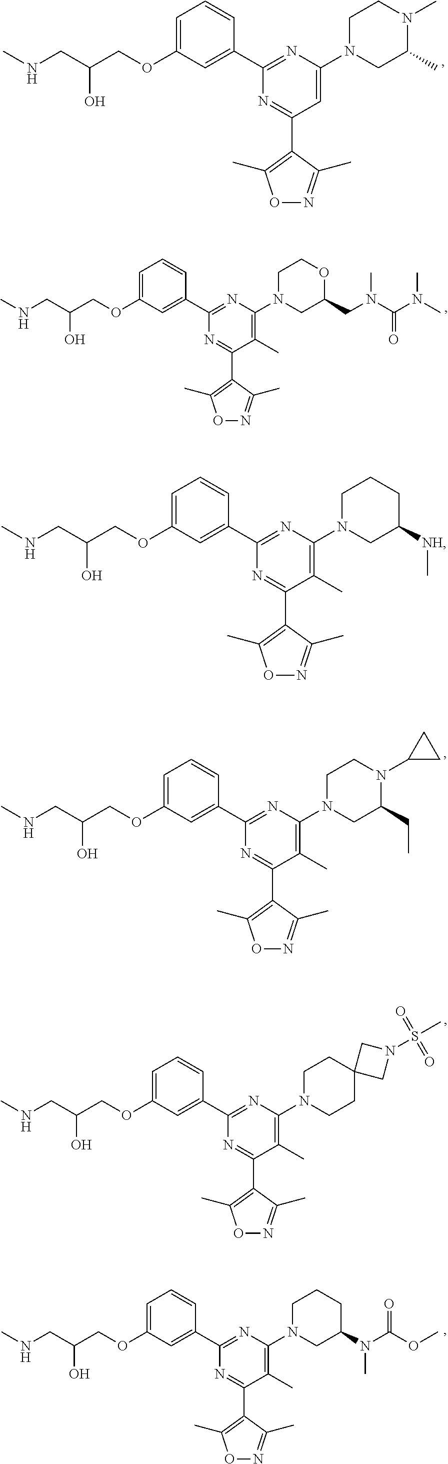

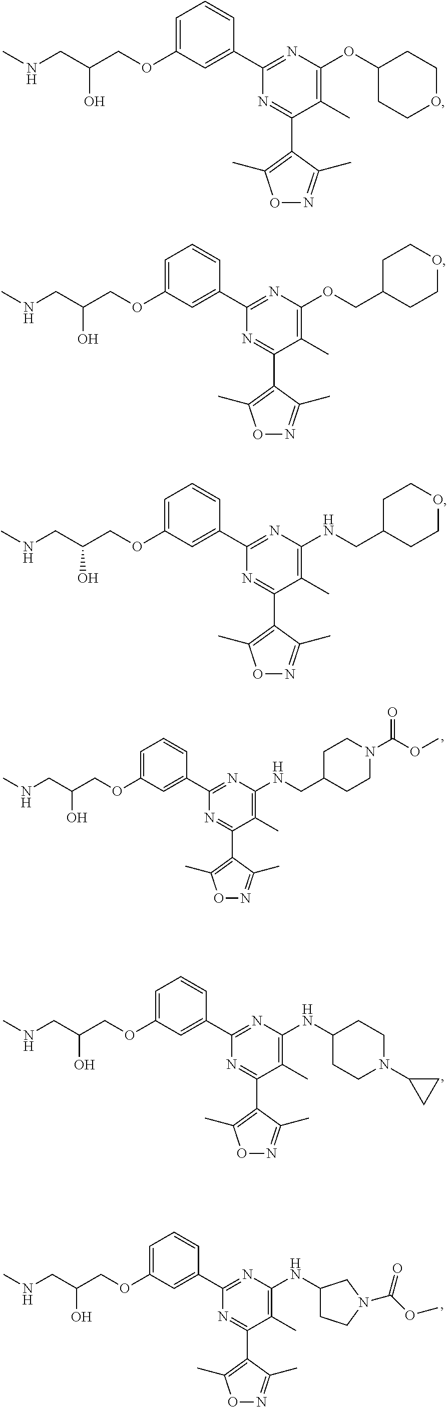

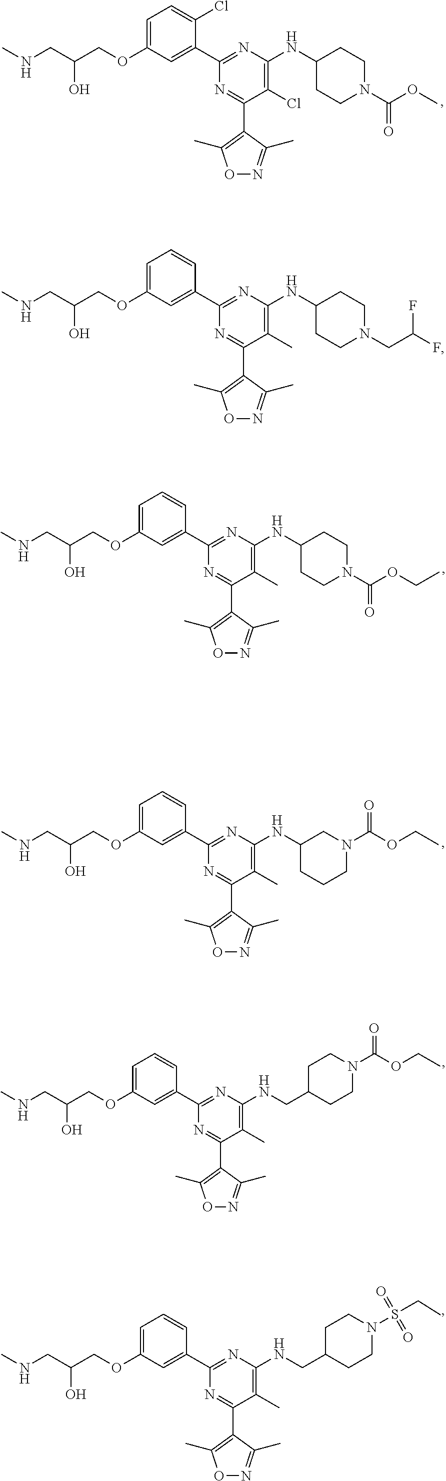

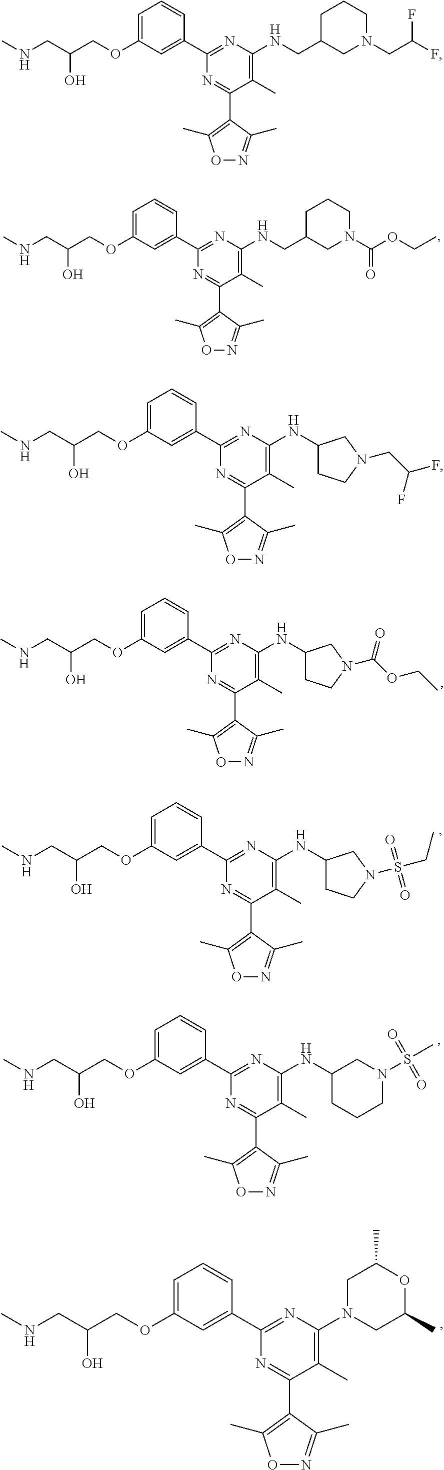

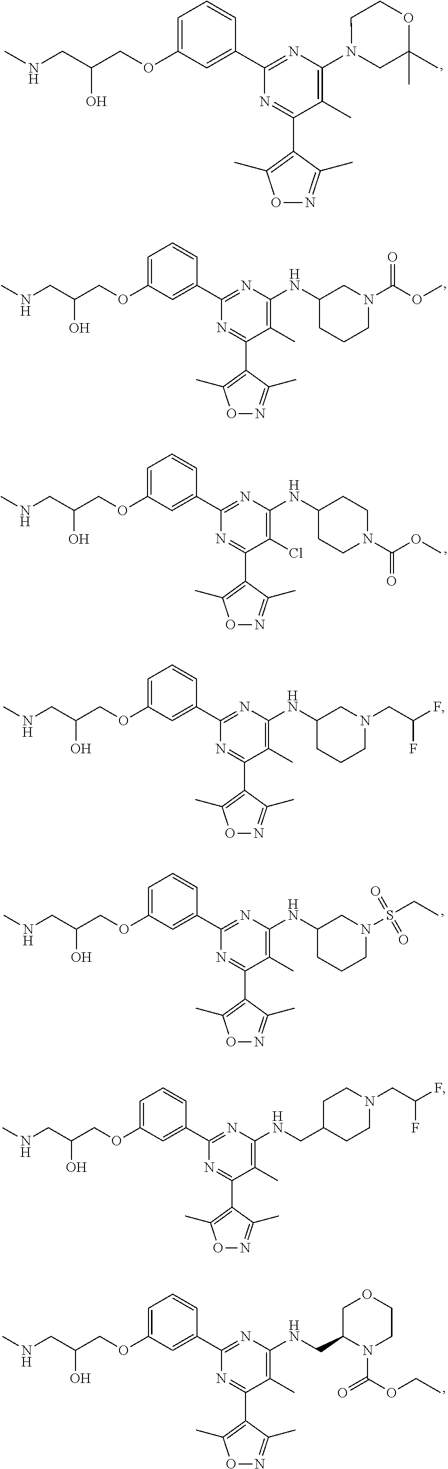

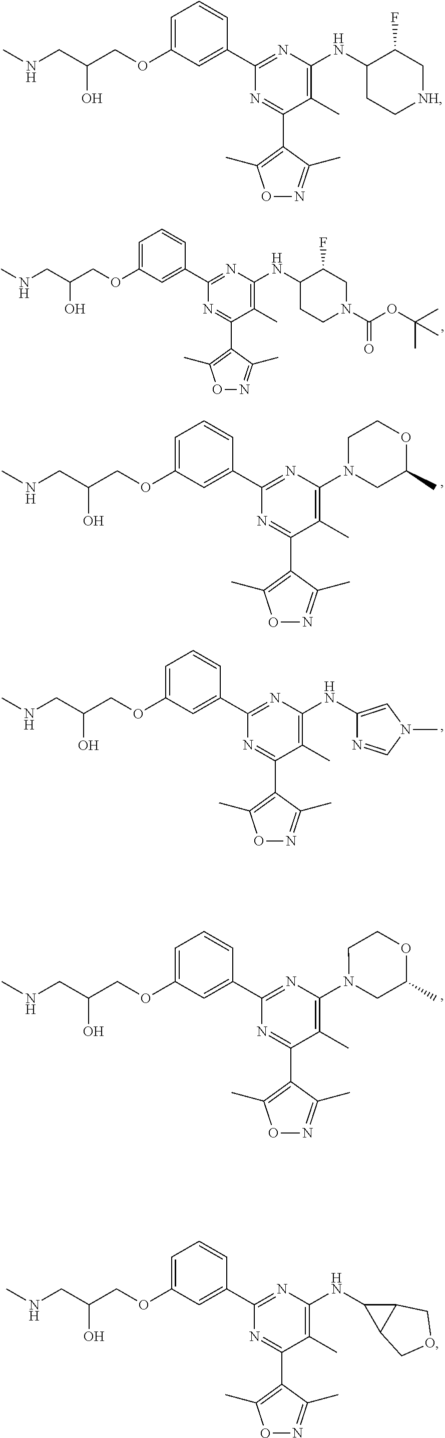

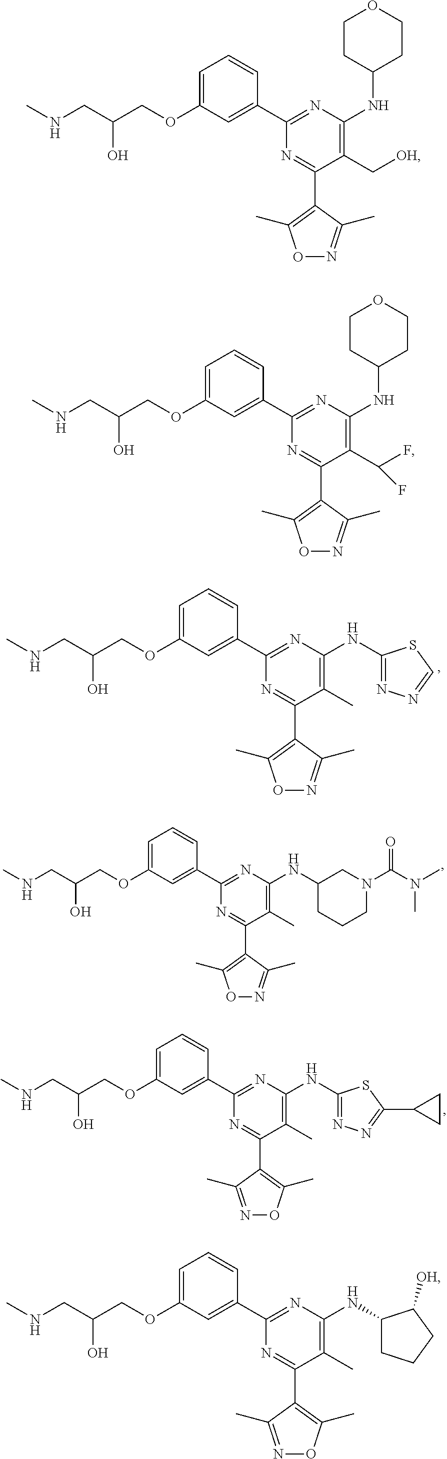

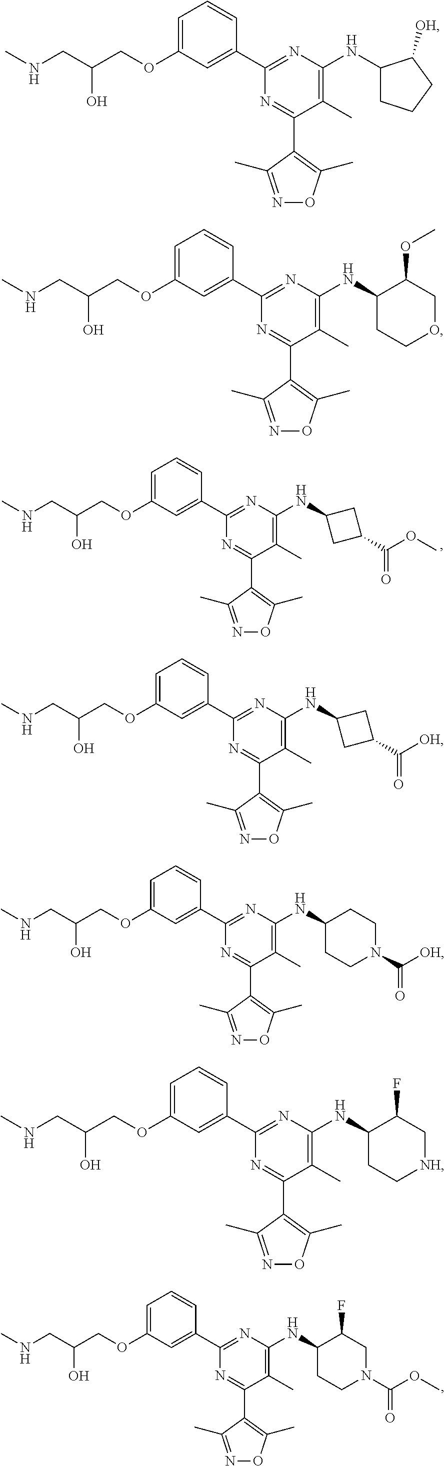

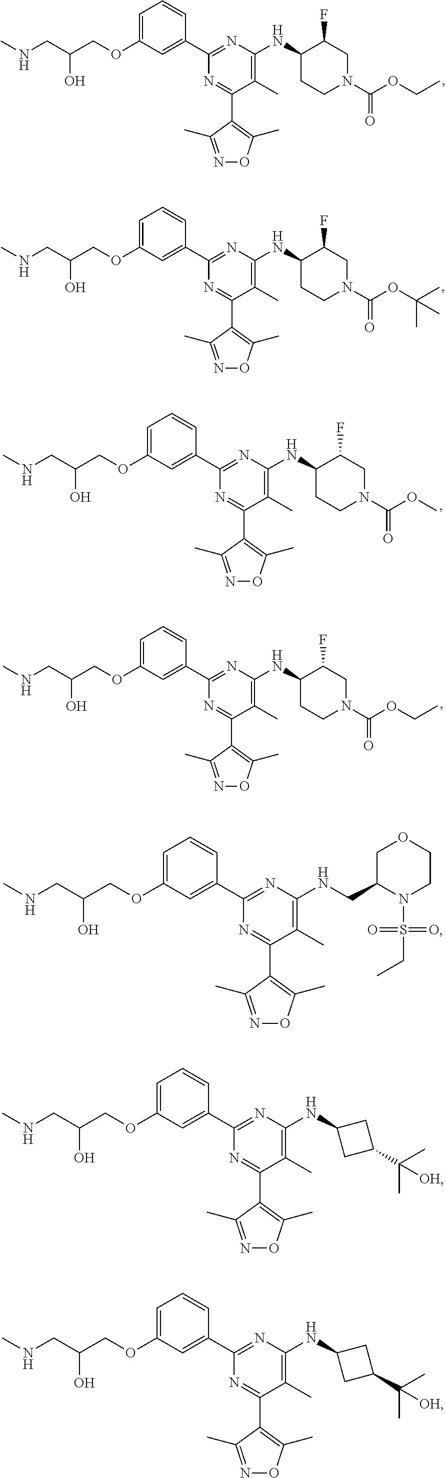

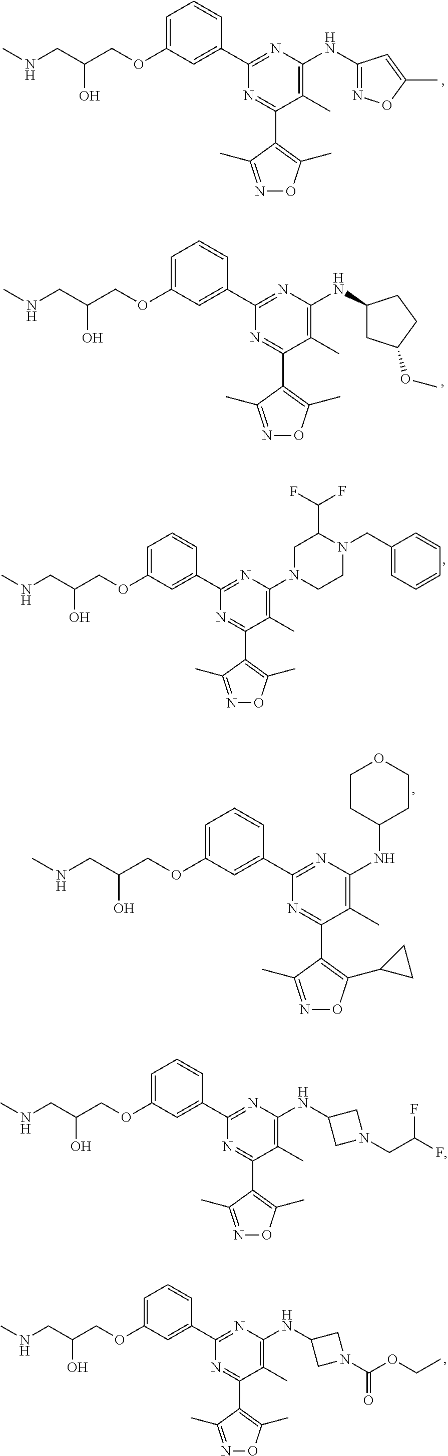

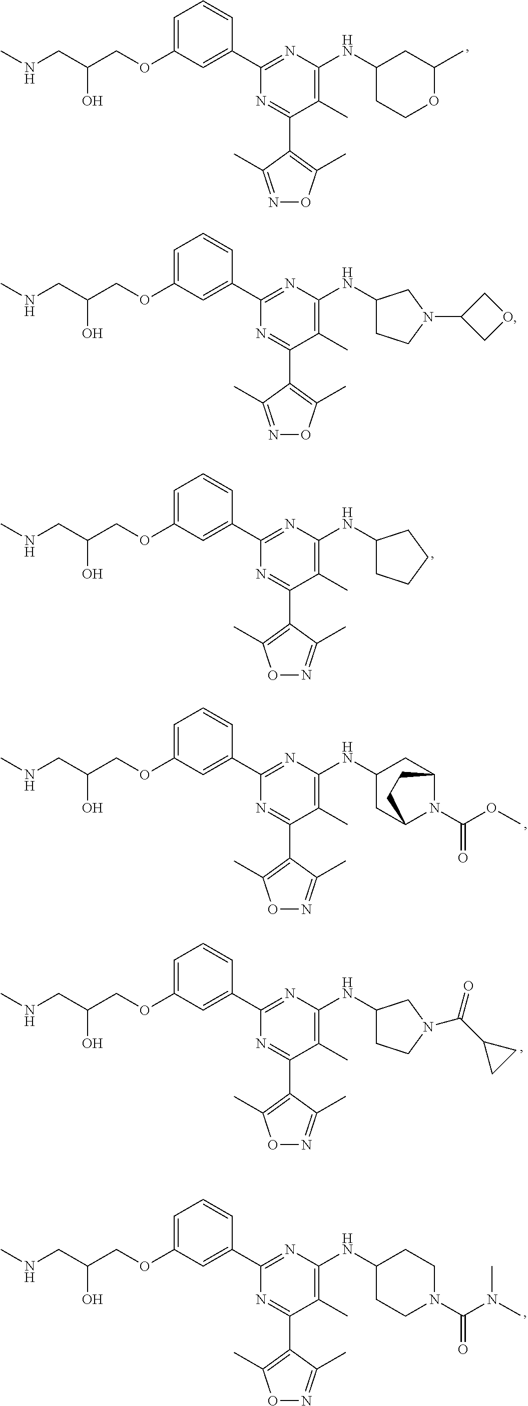

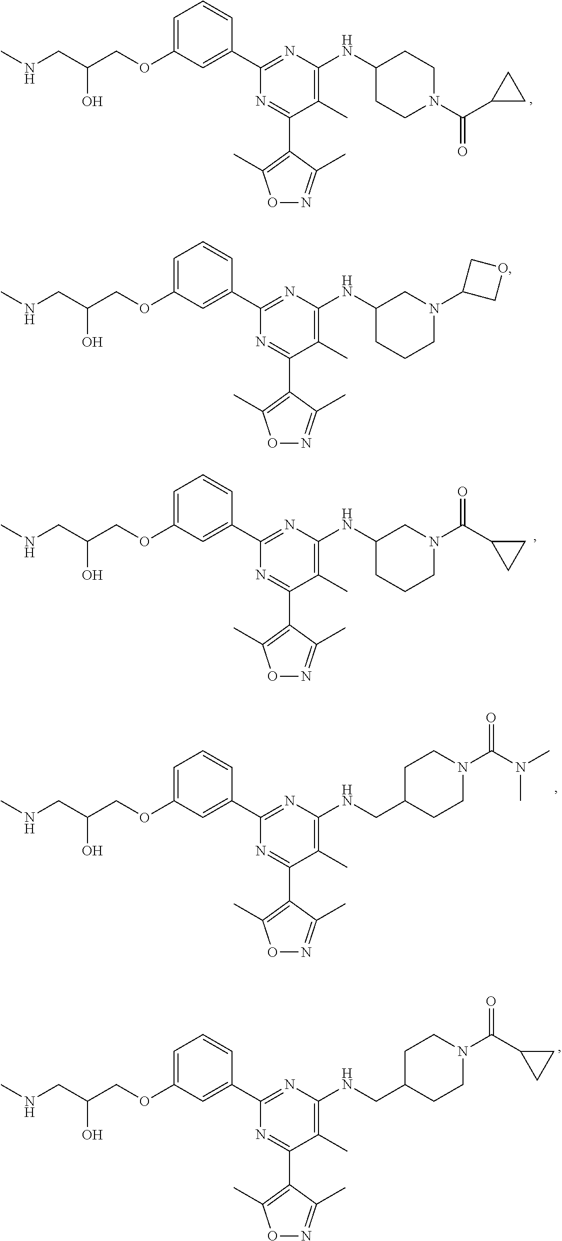

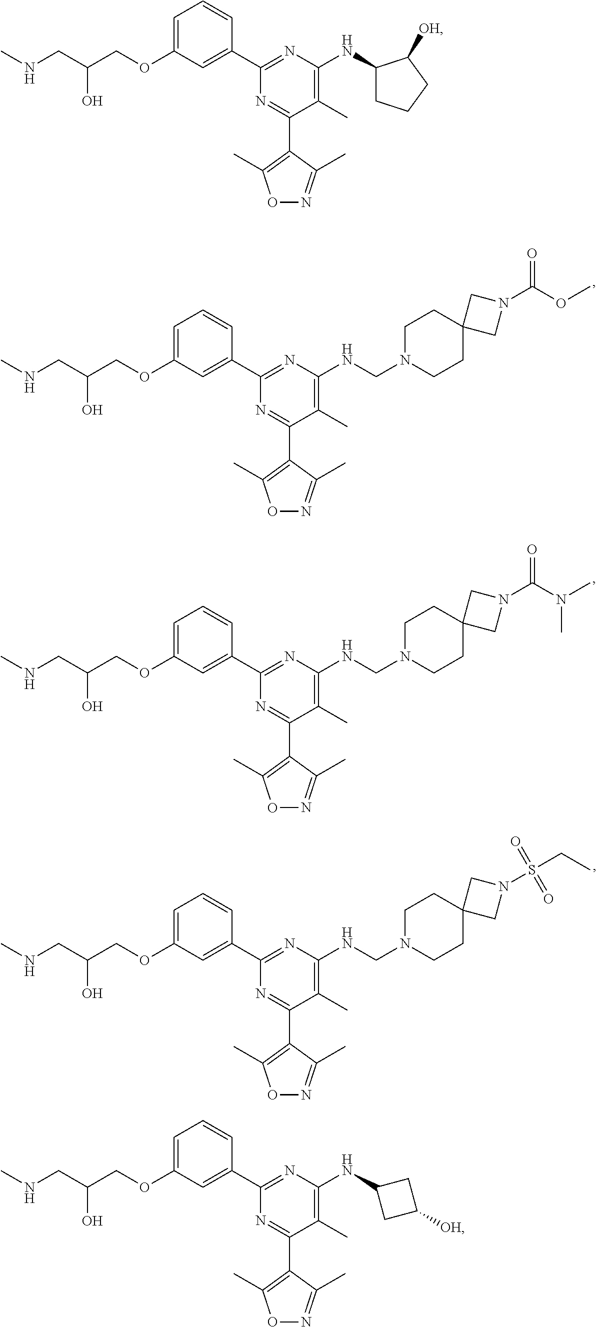

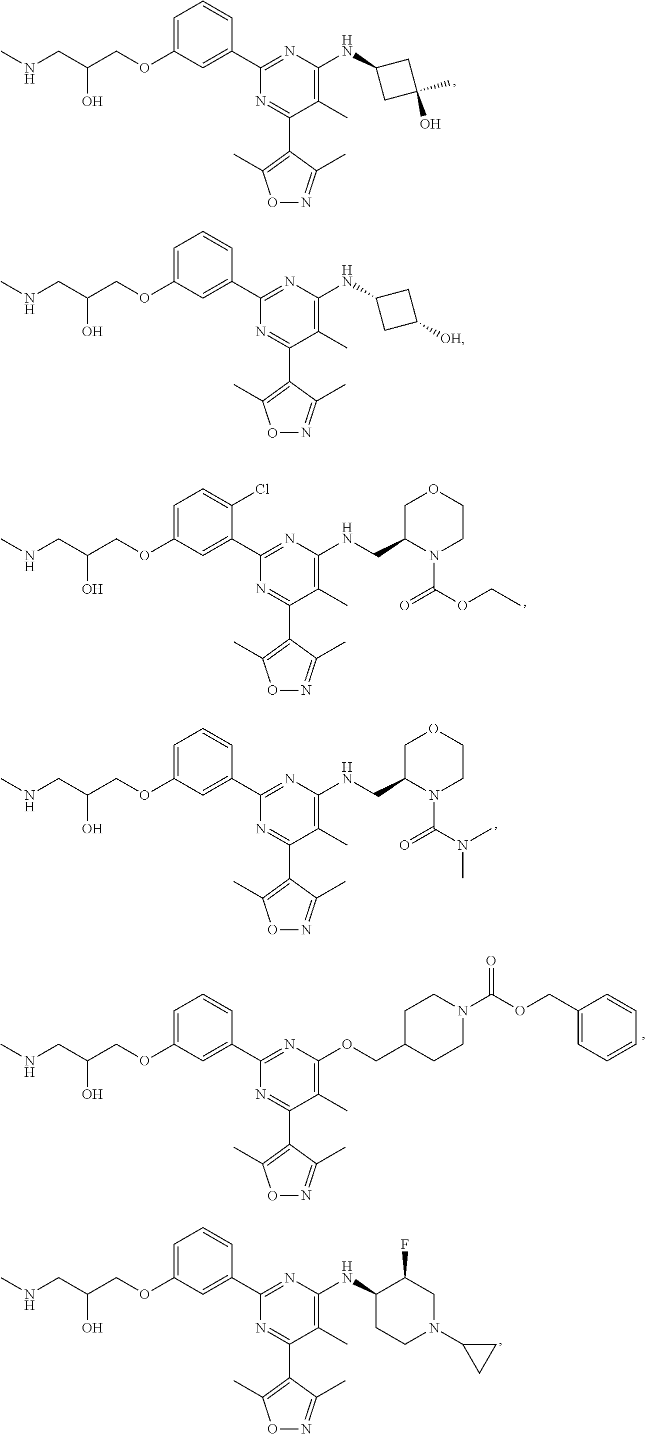

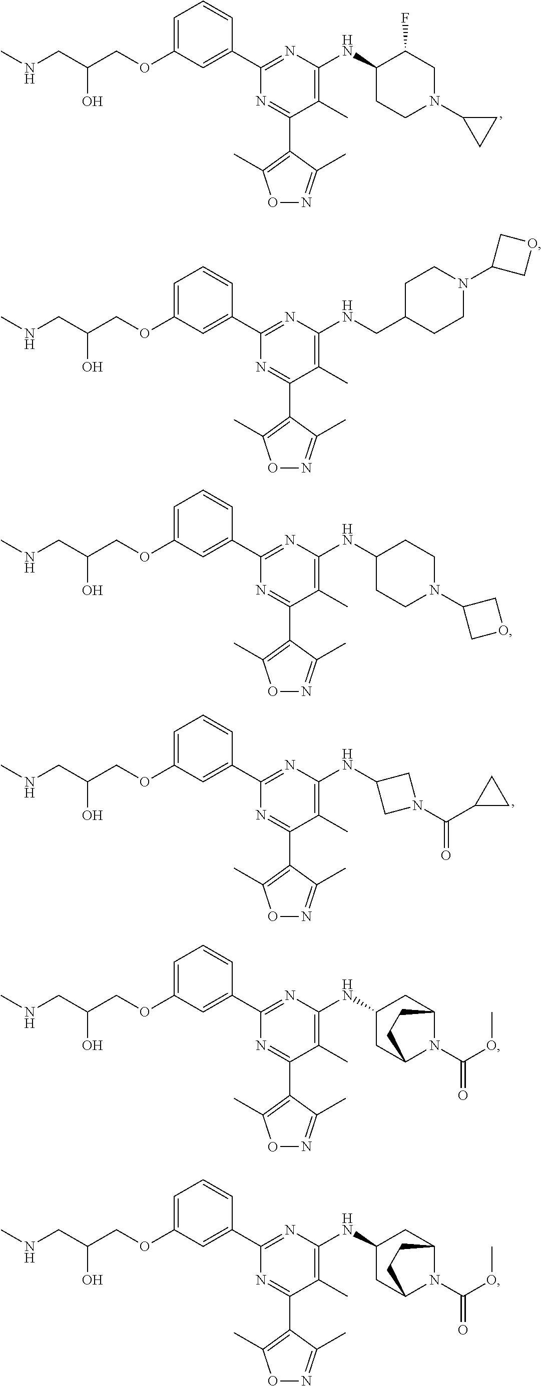

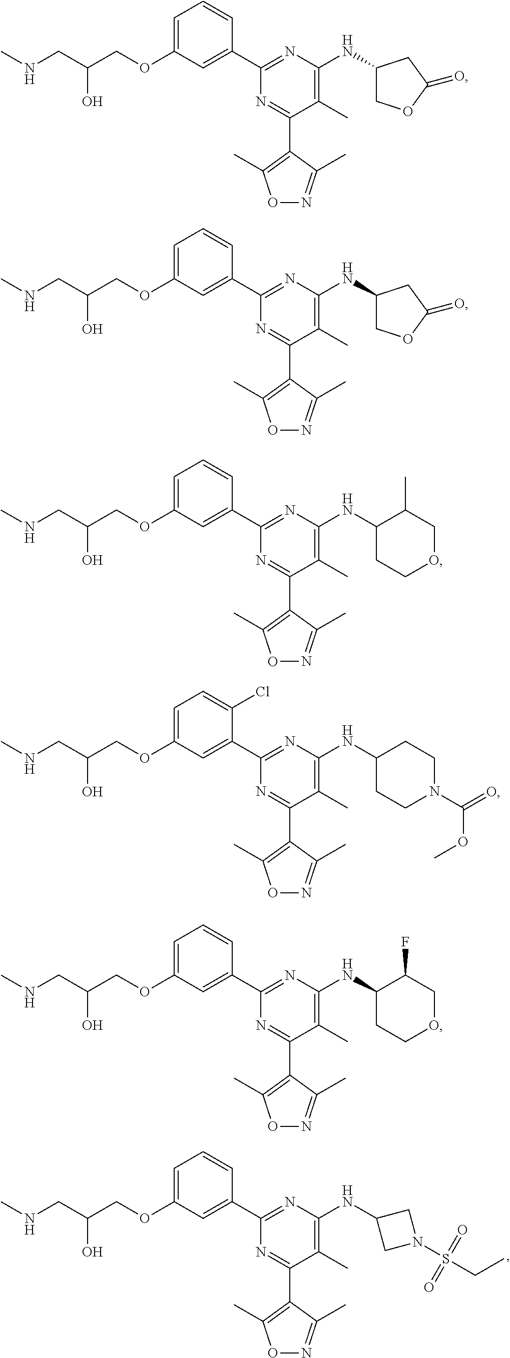

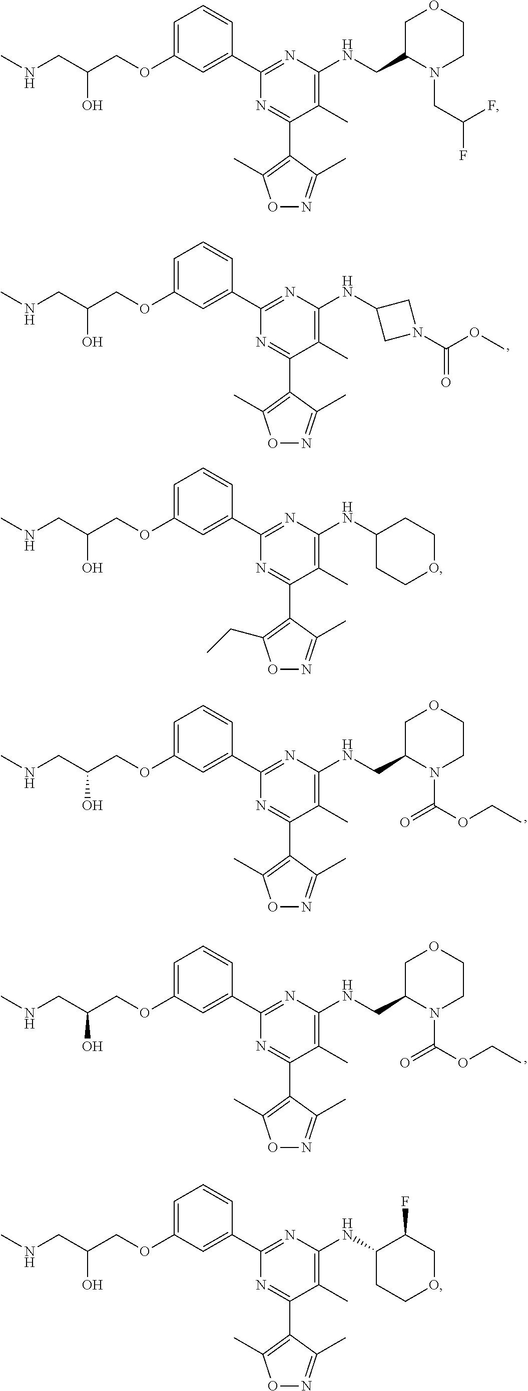

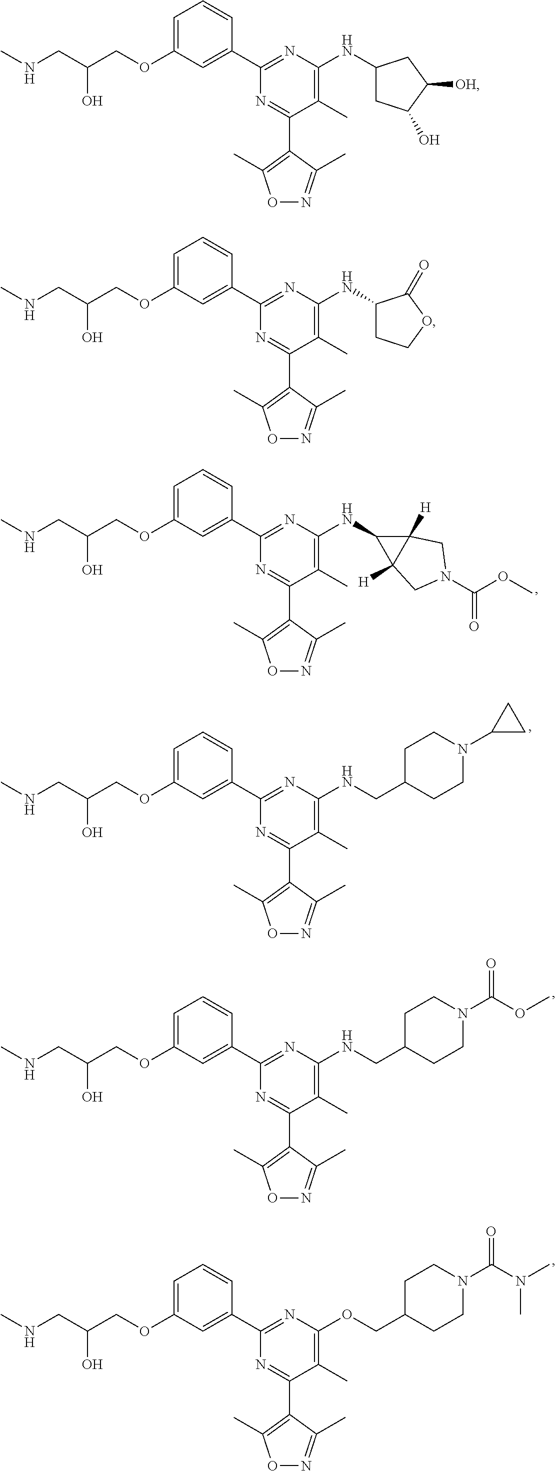

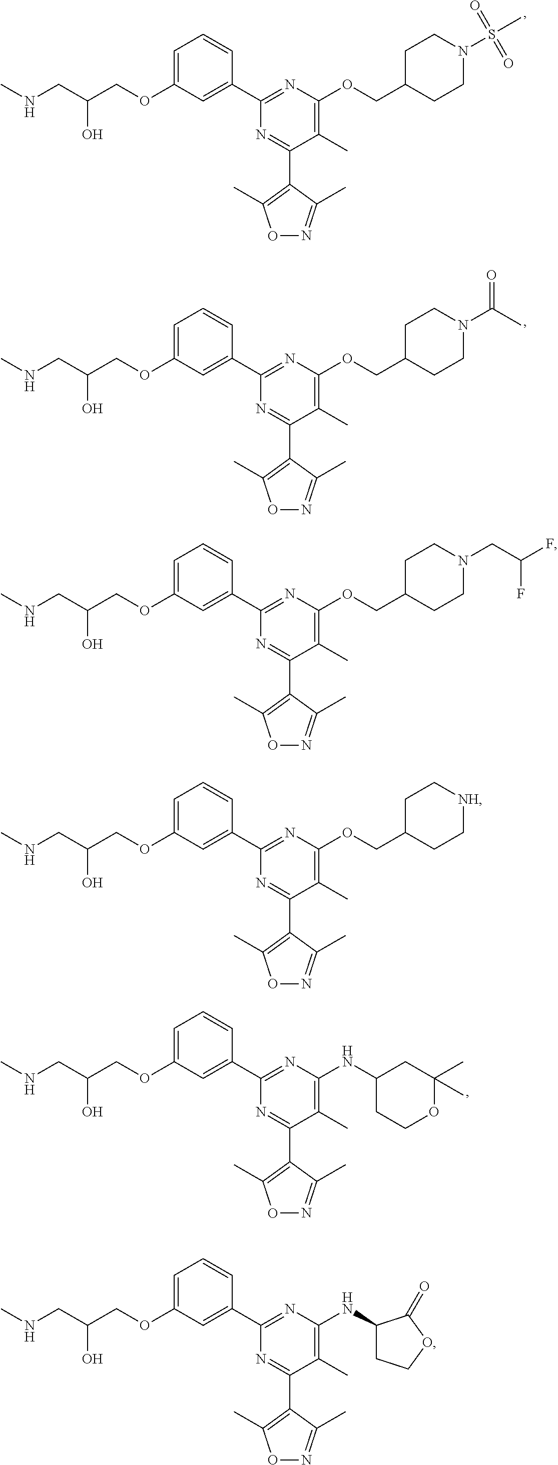

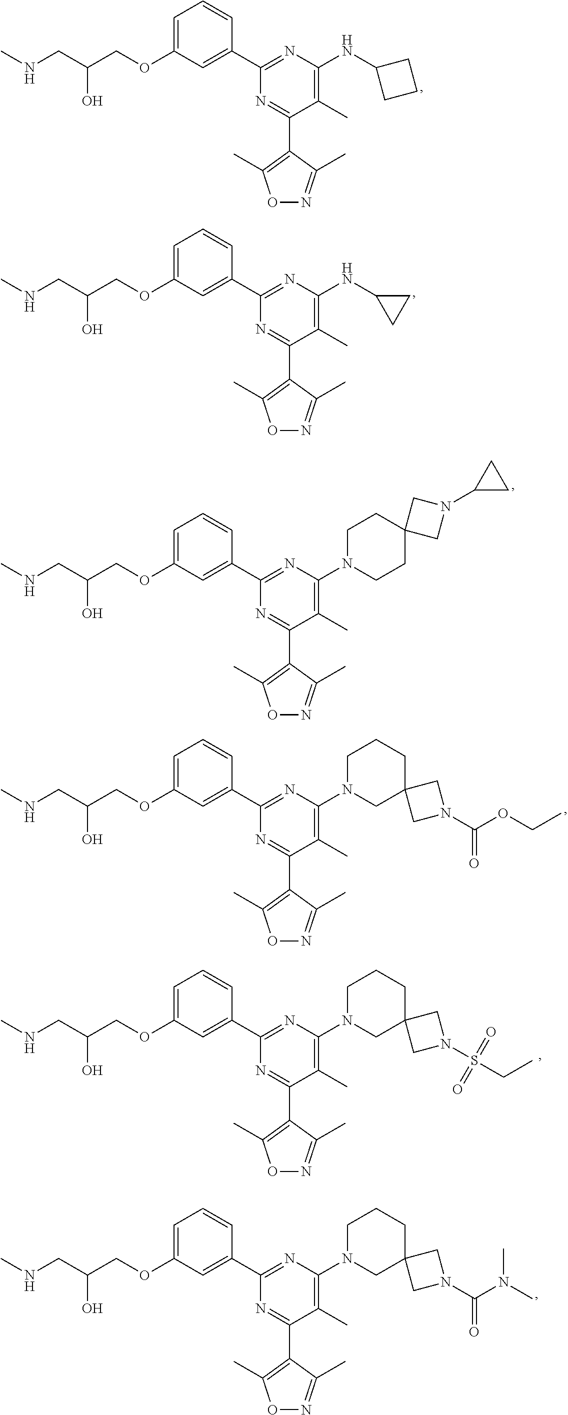

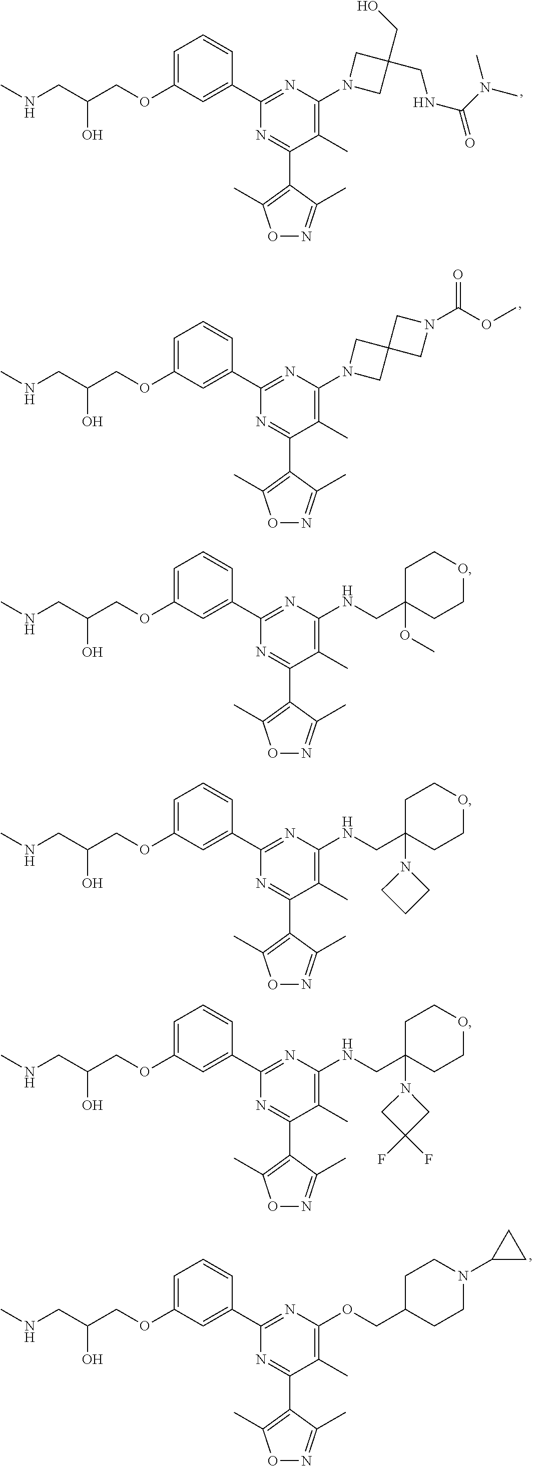

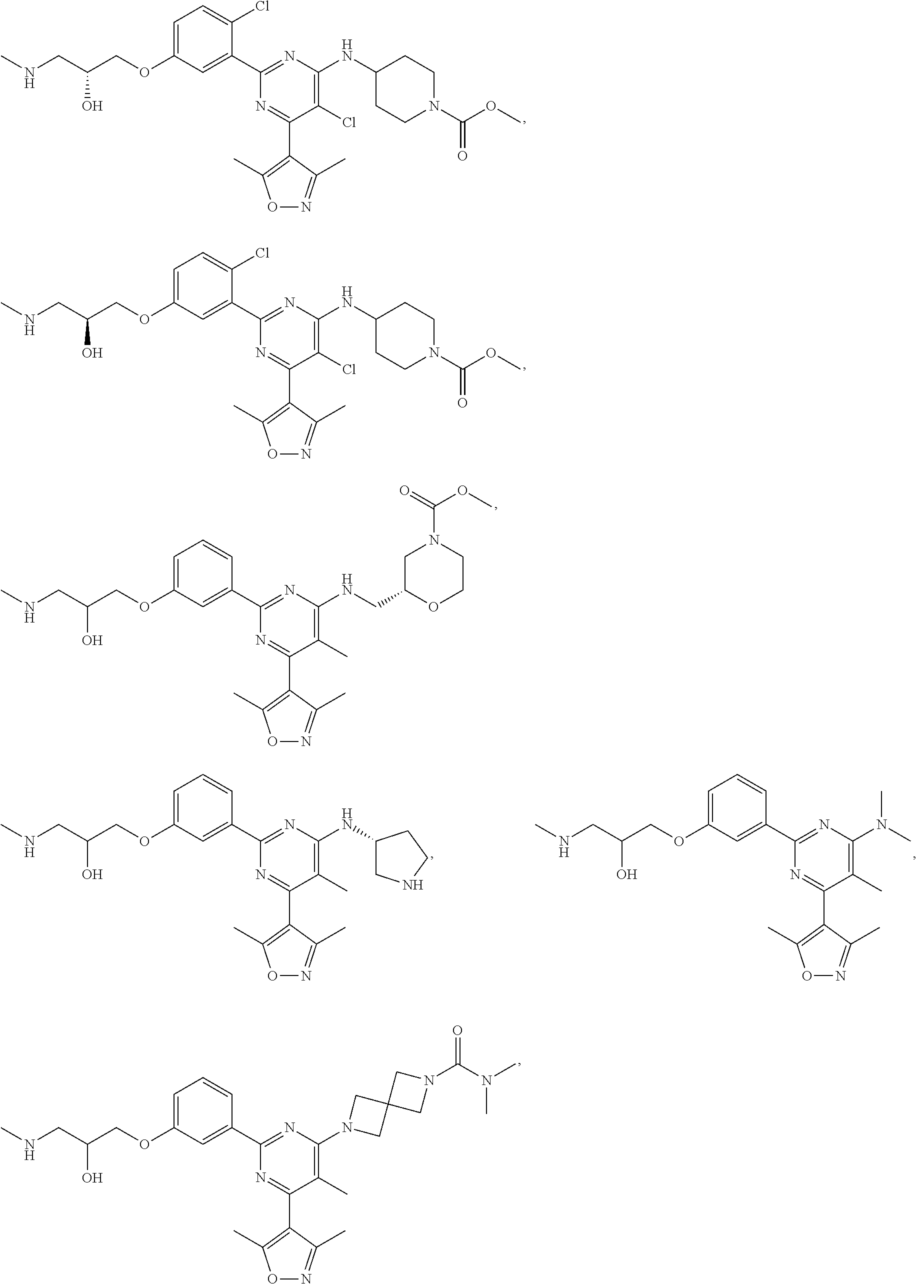

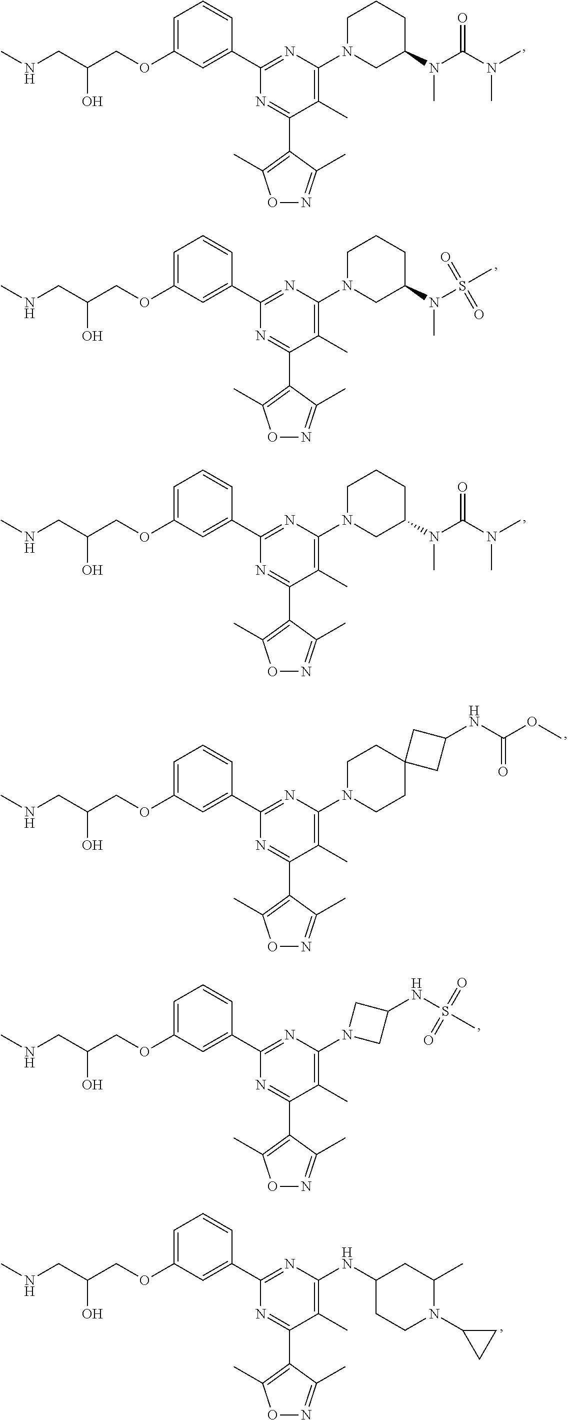

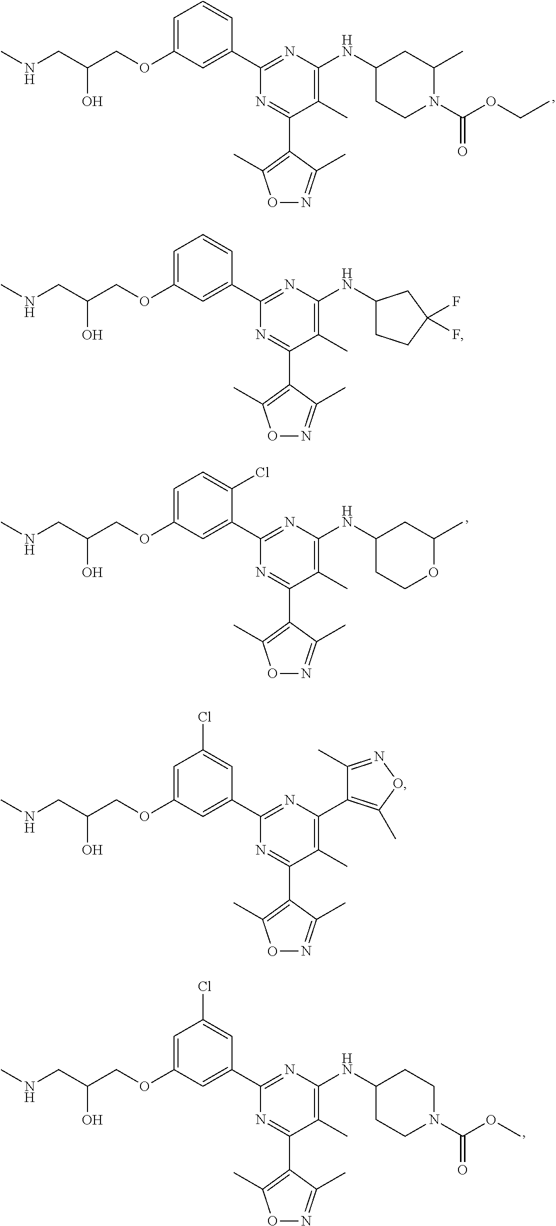

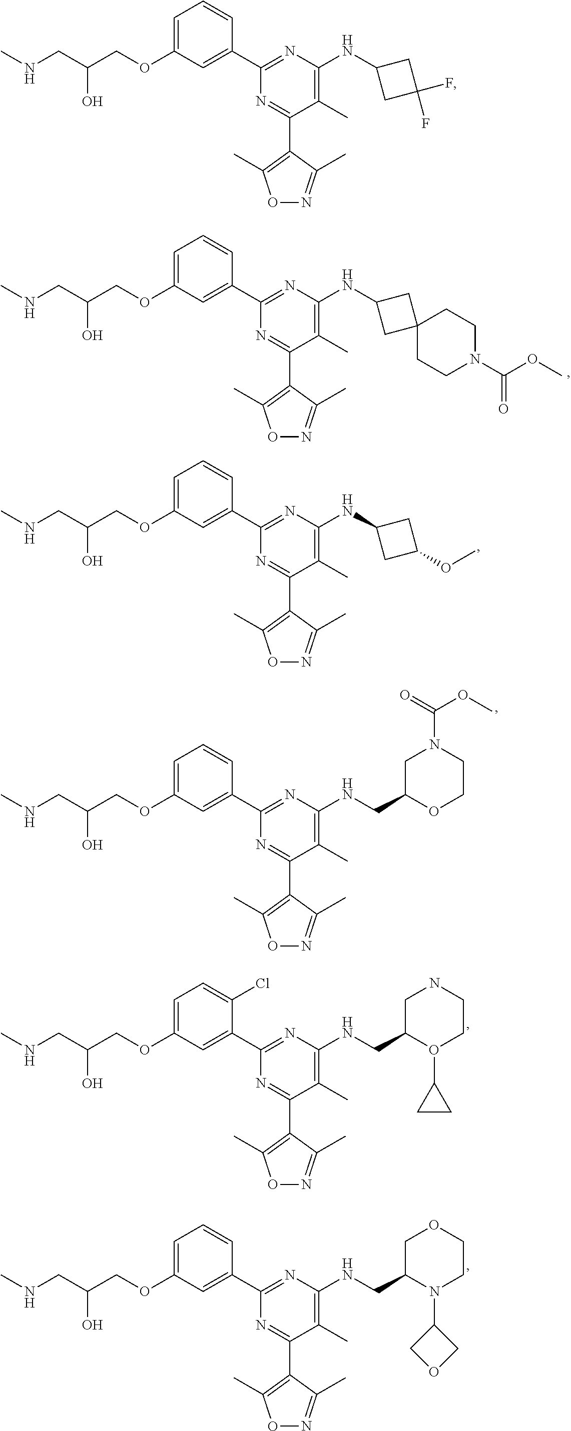

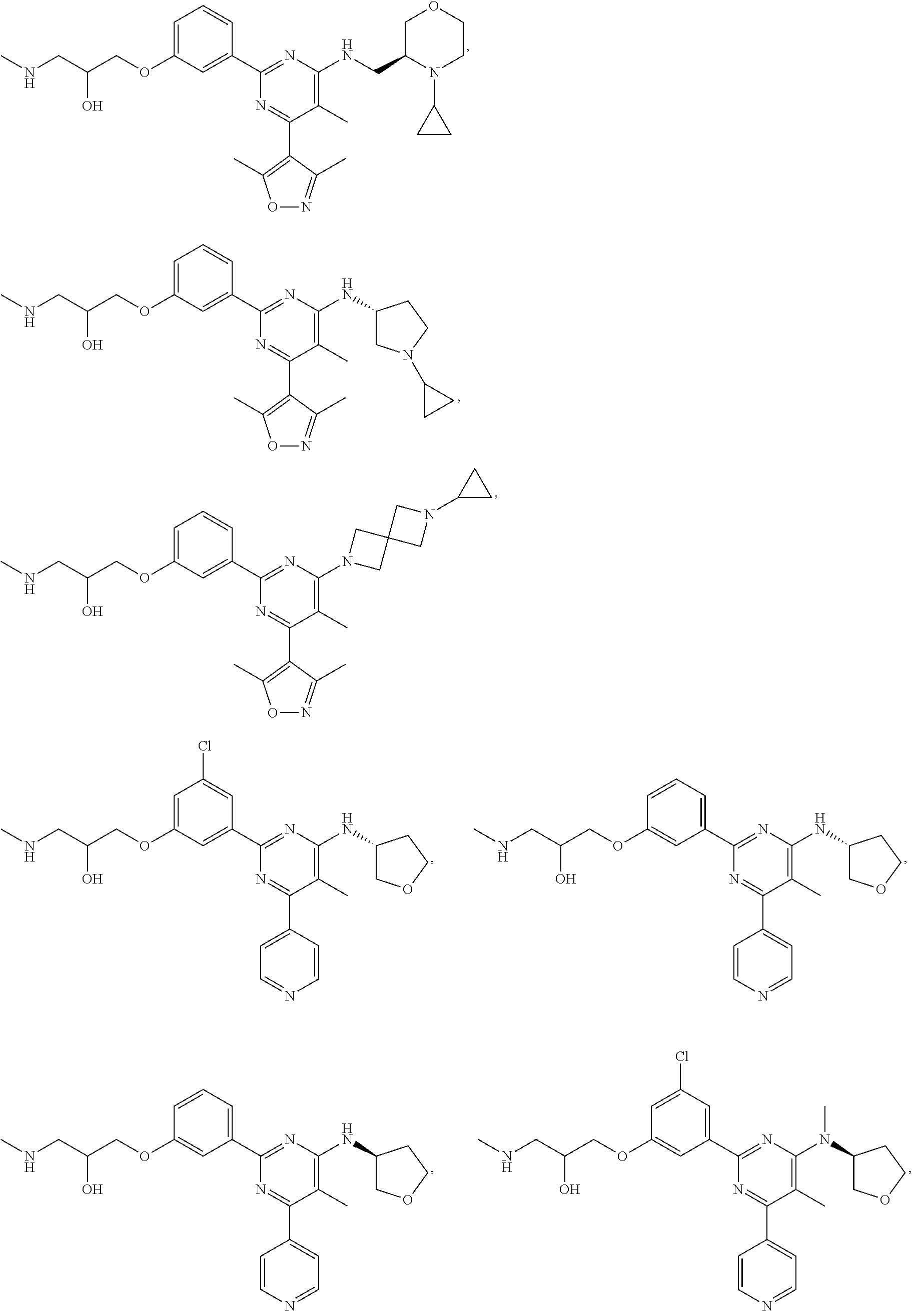

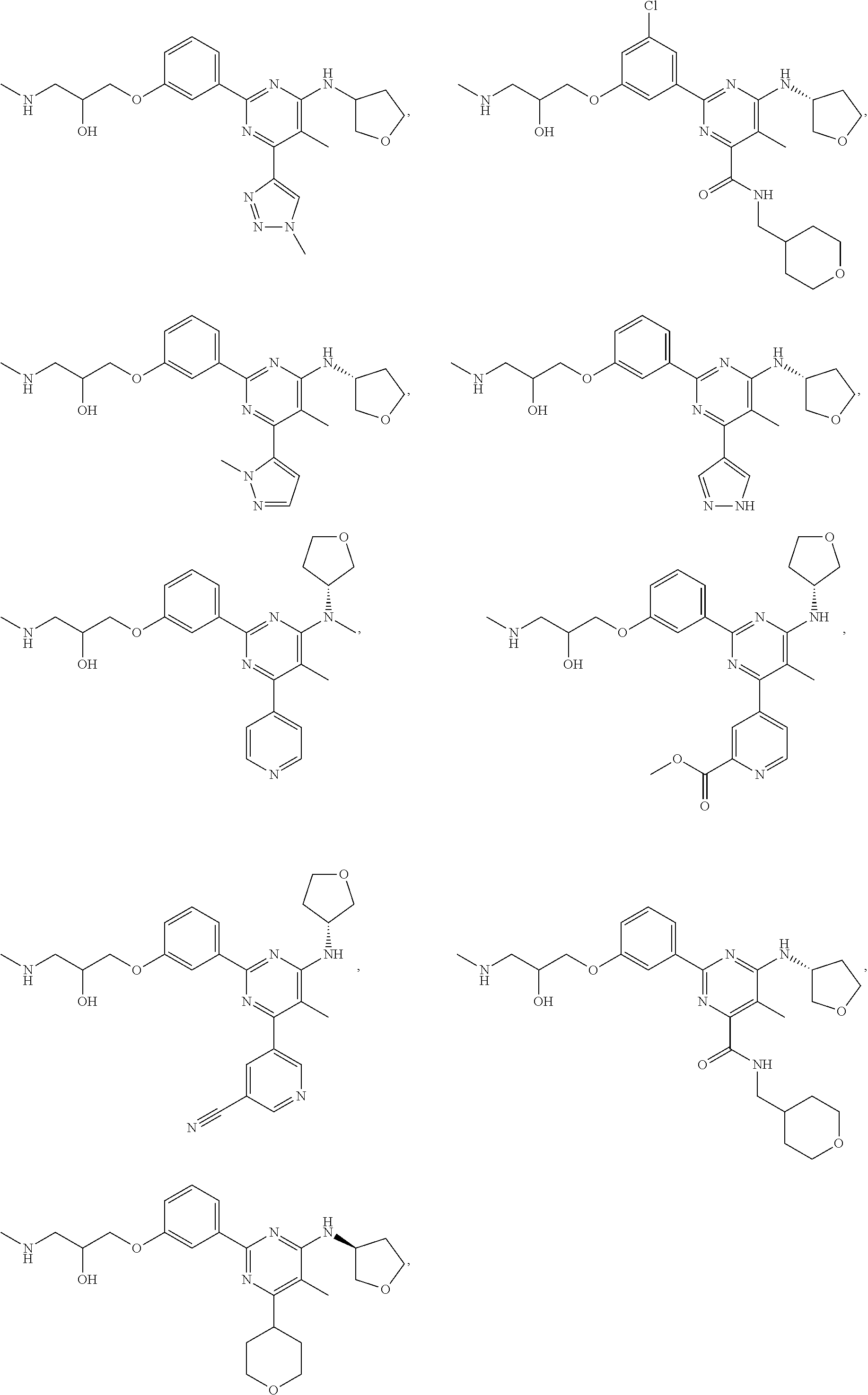

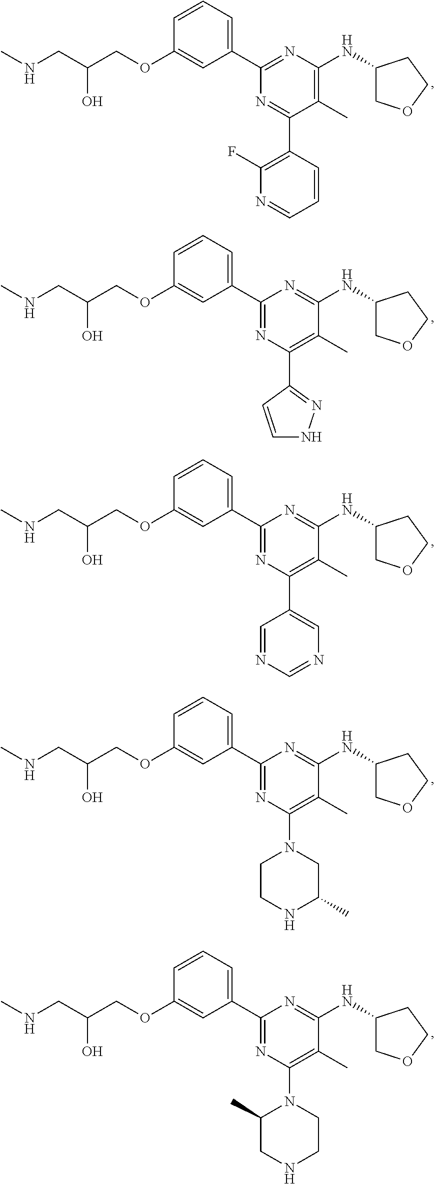 [SMILES: CNCC(O)COc1cccc(-c2nc(N[C@@H]3CCOC3)c(C)c(-c3cccnc3F)n2)c1 ; CNCC(O)COc1cccc(-c2nc(N[C@@H]3CCOC3)c(C)c(-c3cc[nH]n3)n2)c1 ; CNCC(O)COc1cccc(-c2nc(N[C@@H]3CCOC3)c(C)c(-c3cncnc3)n2)c1 ; CNCC(O)COc1cccc(-c2nc(N[C@@H]3CCOC3)c(C)c(N3CCN[C@@H](C)C3)n2)c1 ; CNCC(O)COc1cccc(-c2nc(N[C@@H]3CCOC3)c(C)c(N3CCNC[C@H]3C)n2)c1]

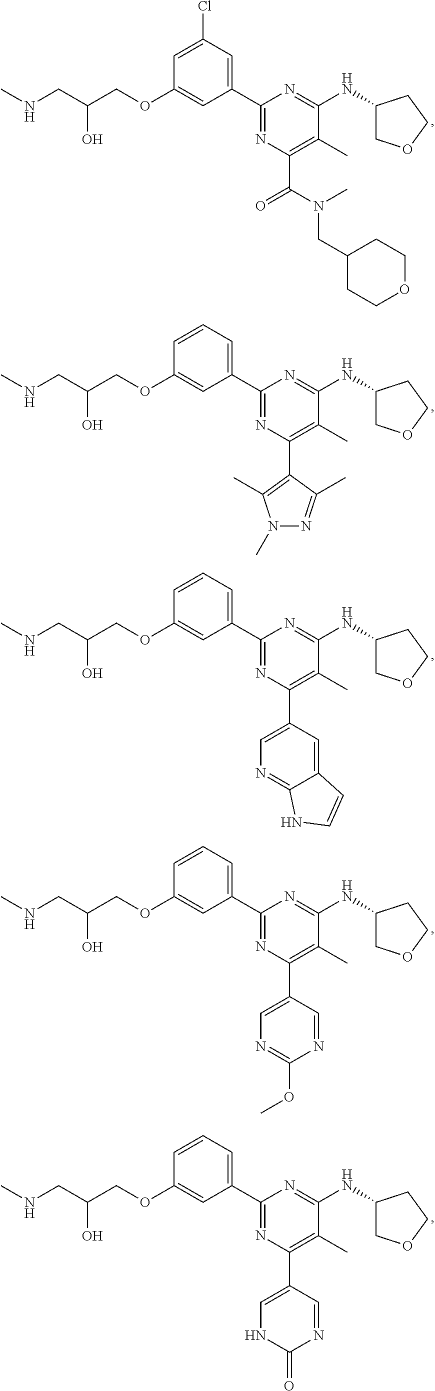

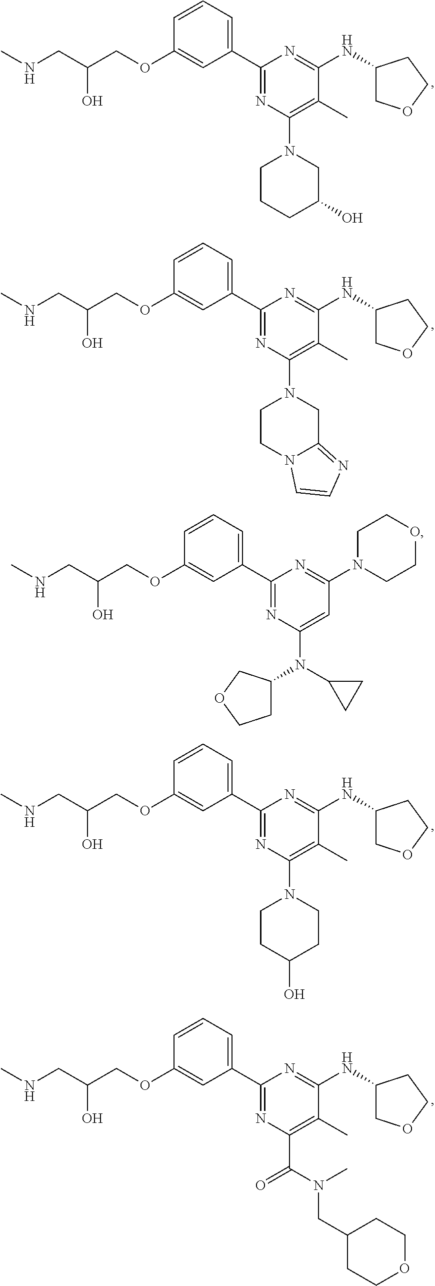

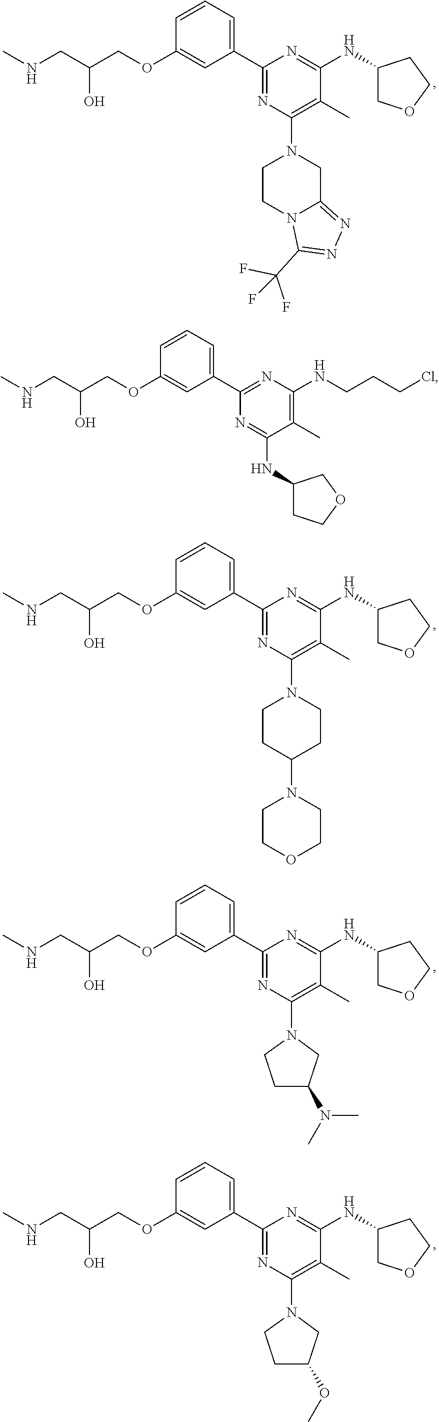 [SMILES: CNCC(O)COc1cccc(-c2nc(N[C@@H]3CCOC3)c(C)c(N3CCn4c(nnc4C(F)(F)F)C3)n2)c1 ; CNCC(O)COc1cccc(-c2nc(NCCCCl)c(C)c(N[C@@H]3CCOC3)n2)c1 ; CNCC(O)COc1cccc(-c2nc(N[C@@H]3CCOC3)c(C)c(N3CCC(N4CCOCC4)CC3)n2)c1 ; CNCC(O)COc1cccc(-c2nc(N[C@@H]3CCOC3)c(C)c(N3CC[C@H](N(C)C)C3)n2)c1 ; CNCC(O)COc1cccc(-c2nc(N[C@@H]3CCOC3)c(C)c(N3CC[C@@H](OC)C3)n2)c1]

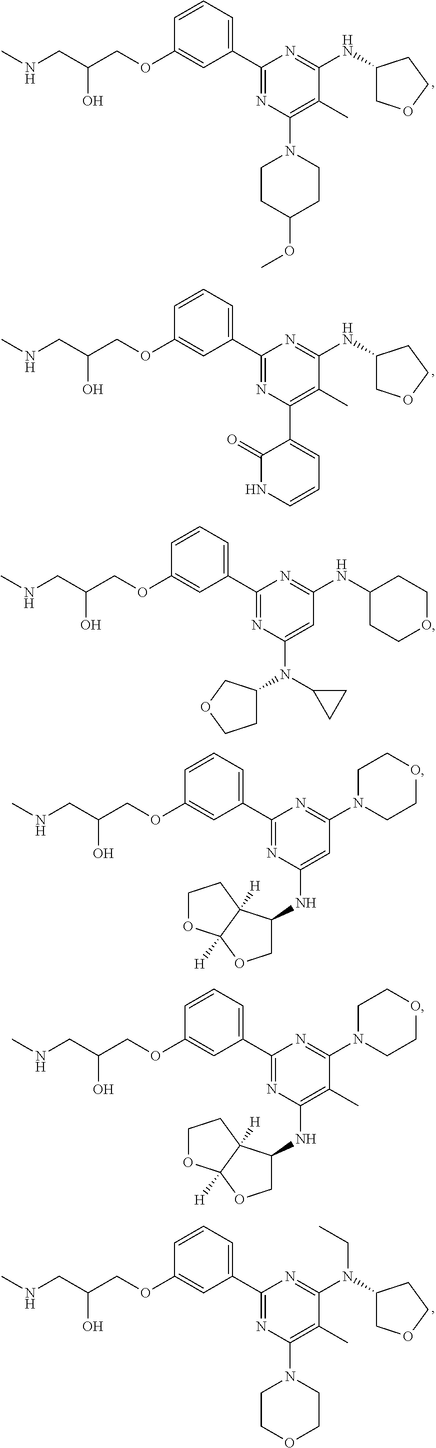

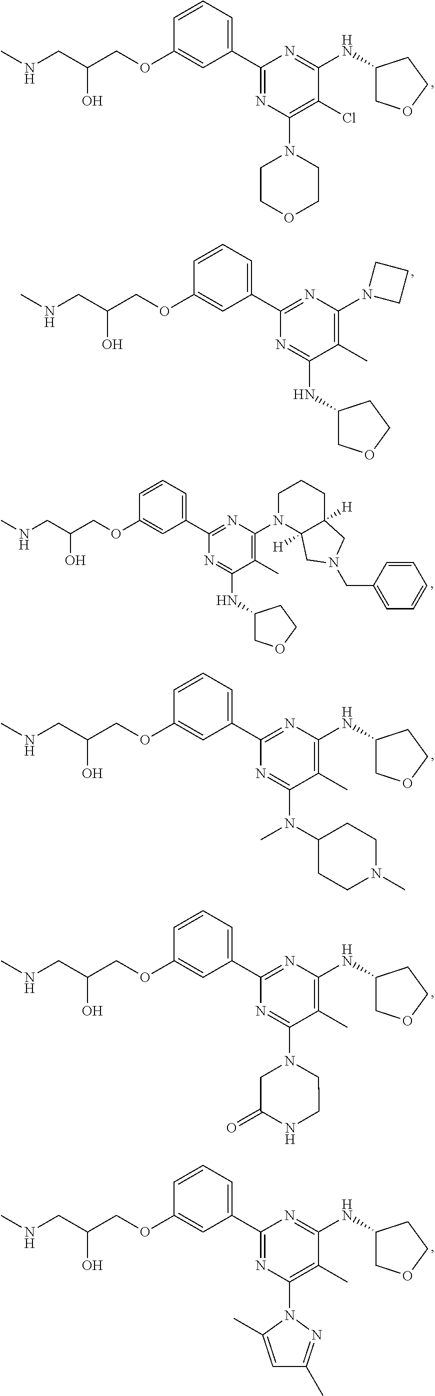 [SMILES: CNCC(O)COc1cccc(-c2nc(N[C@@H]3CCOC3)c(Cl)c(N3CCOCC3)n2)c1 ; CNCC(O)COc1cccc(-c2nc(N[C@@H]3CCOC3)c(C)c(N3CCC3)n2)c1 ; CNCC(O)COc1cccc(-c2nc(N[C@@H]3CCOC3)c(C)c(N3CCC[C@H]4CN(Cc5ccccc5)C[C@H]43)n2)c1 ; CNCC(O)COc1cccc(-c2nc(N[C@@H]3CCOC3)c(C)c(N(C)C3CCN(C)CC3)n2)c1 ; CNCC(O)COc1cccc(-c2nc(N[C@@H]3CCOC3)c(C)c(N3CCNC(=O)C3)n2)c1 ; CNCC(O)COc1cccc(-c2nc(N[C@@H]3CCOC3)c(C)c(-n3nc(C)cc3C)n2)c1]

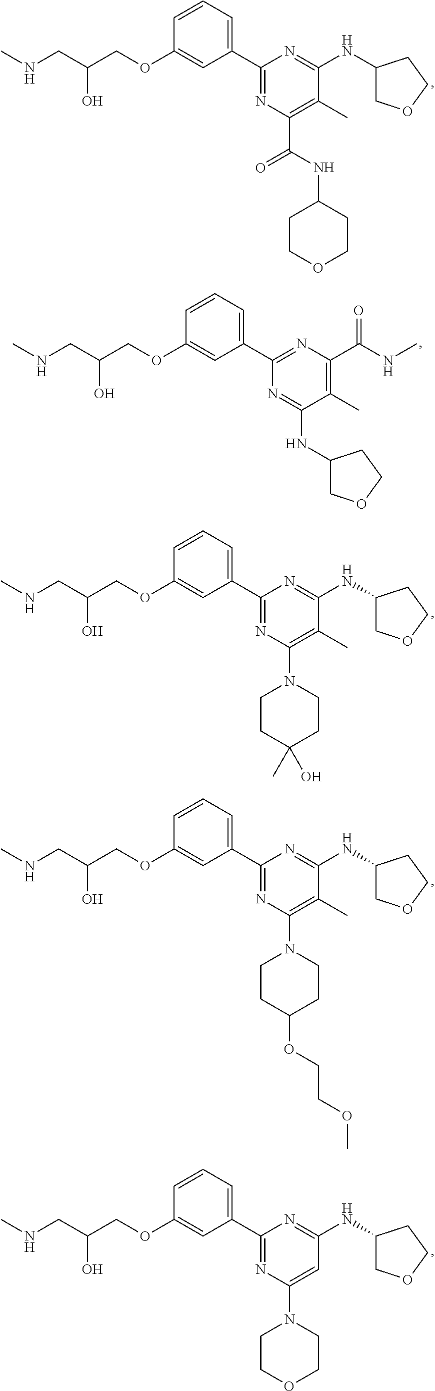

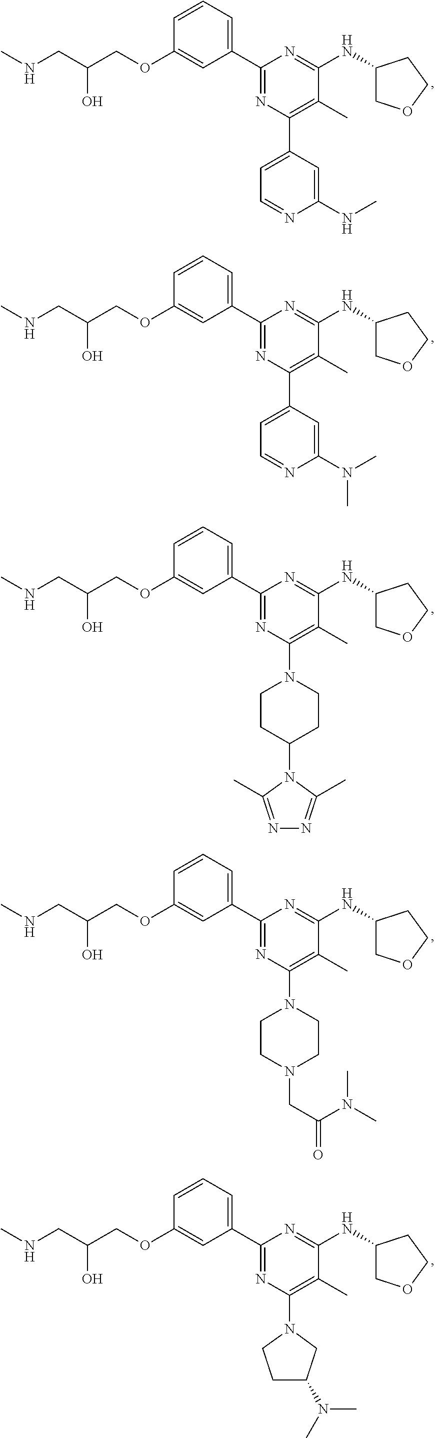 [SMILES: CNCC(O)COc1cccc(-c2nc(N[C@@H]3CCOC3)c(C)c(-c3ccnc(NC)c3)n2)c1 ; CNCC(O)COc1cccc(-c2nc(N[C@@H]3CCOC3)c(C)c(-c3ccnc(N(C)C)c3)n2)c1 ; CNCC(O)COc1cccc(-c2nc(N[C@@H]3CCOC3)c(C)c(N3CCC(n4c(C)nnc4C)CC3)n2)c1 ; CNCC(O)COc1cccc(-c2nc(N[C@@H]3CCOC3)c(C)c(N3CCN(CC(=O)N(C)C)CC3)n2)c1 ; CNCC(O)COc1cccc(-c2nc(N[C@@H]3CCOC3)c(C)c(N3CC[C@@H](N(C)C)C3)n2)c1]

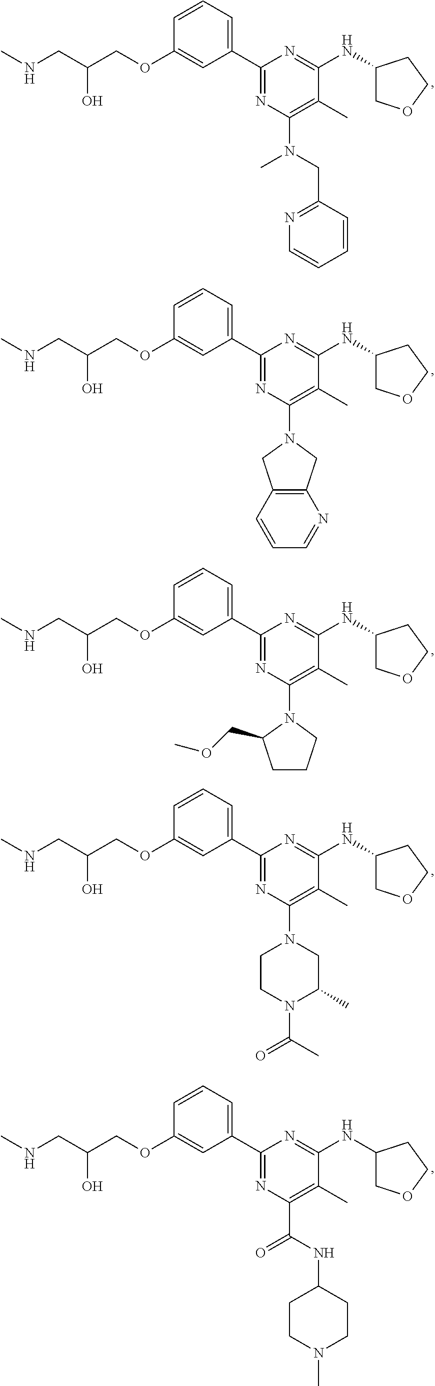

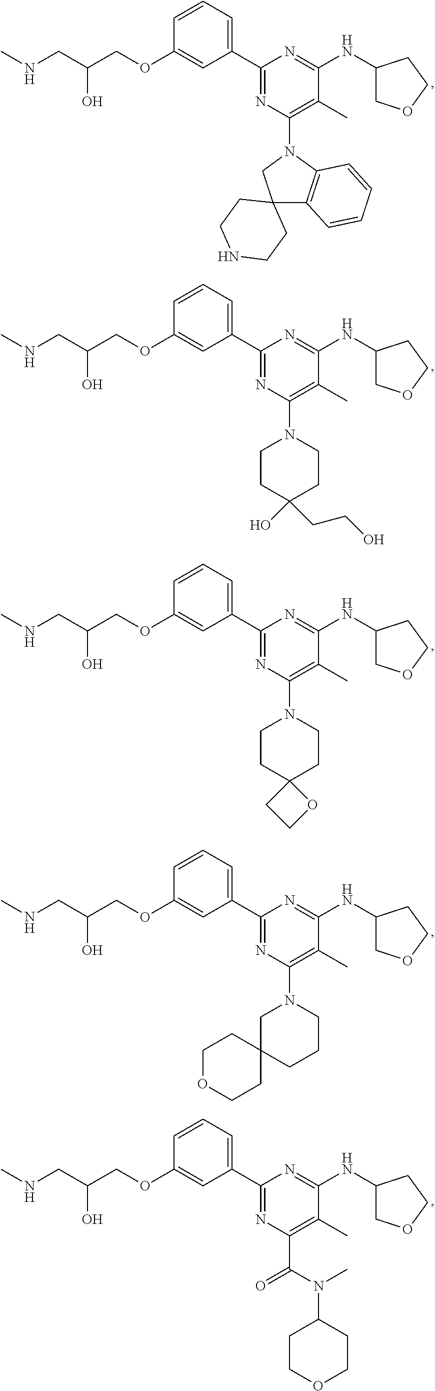

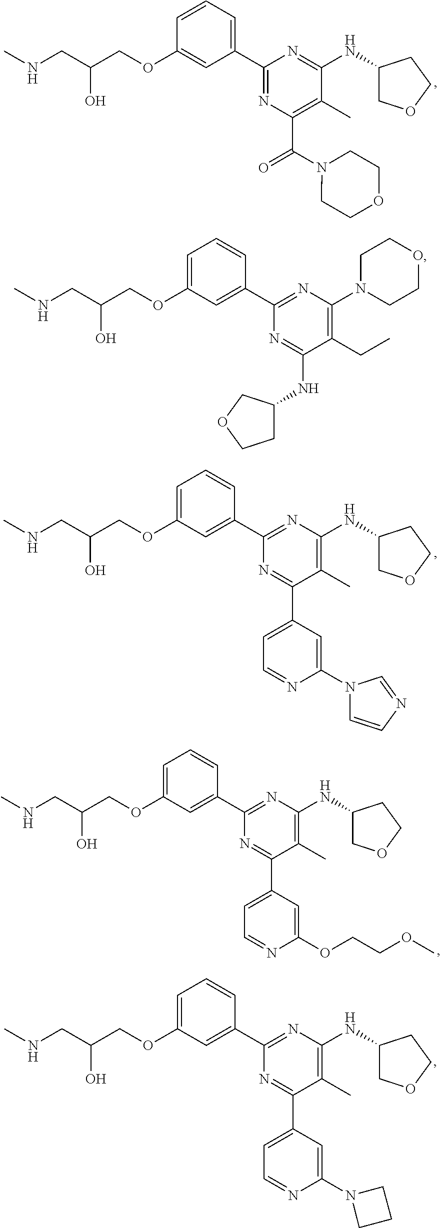

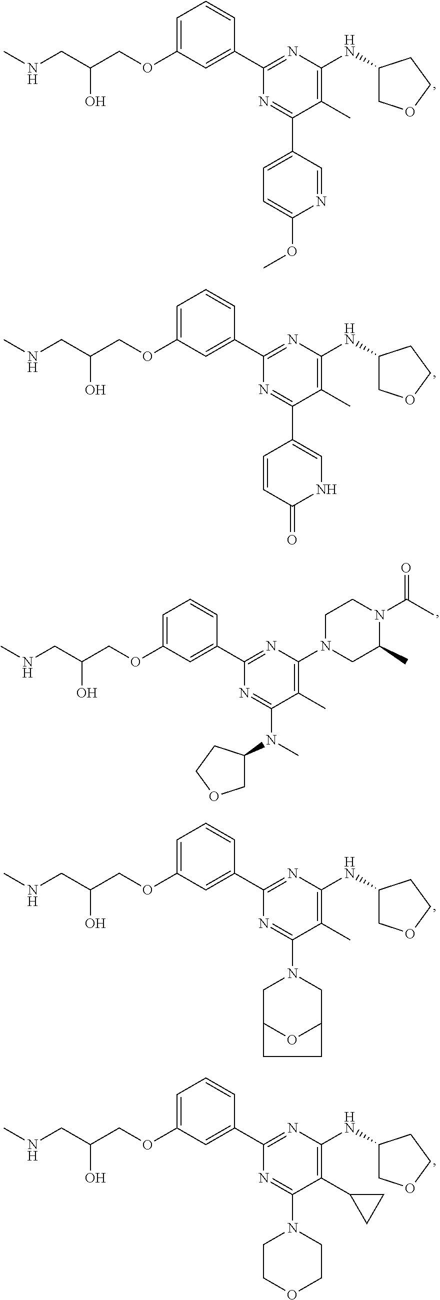

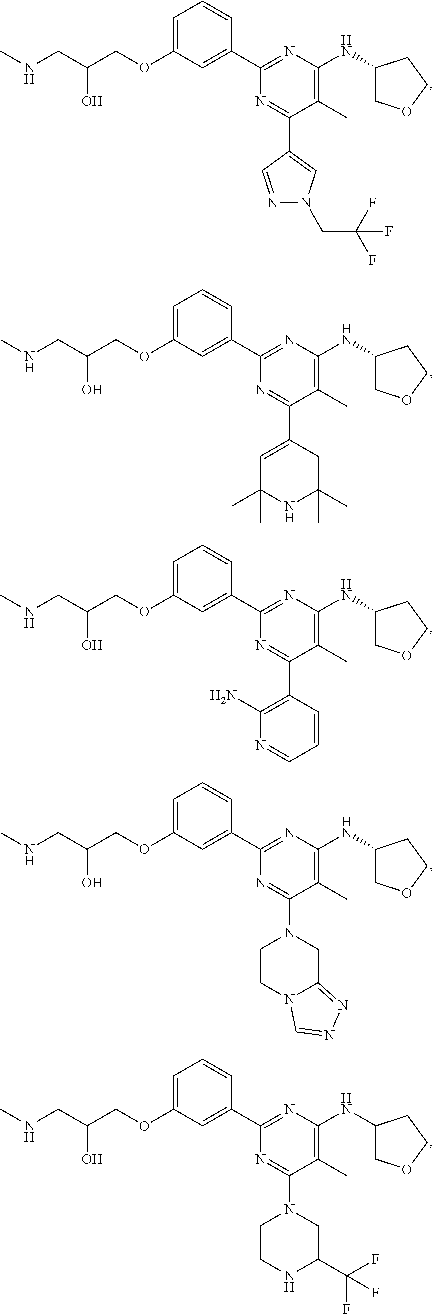 [SMILES: CNCC(O)COc1cccc(-c2nc(N[C@@H]3CCOC3)c(C)c(-c3cnn(CC(F)(F)F)c3)n2)c1 ; CNCC(O)COc1cccc(-c2nc(N[C@@H]3CCOC3)c(C)c(C3=CC(C)(C)NC(C)(C)C3)n2)c1 ; CNCC(O)COc1cccc(-c2nc(N[C@@H]3CCOC3)c(C)c(-c3cccnc3N)n2)c1 ; CNCC(O)COc1cccc(-c2nc(N[C@@H]3CCOC3)c(C)c(N3CCn4cnnc4C3)n2)c1 ; CNCC(O)COc1cccc(-c2nc(NC3CCOC3)c(C)c(N3CCNC(C(F)(F)F)C3)n2)c1]

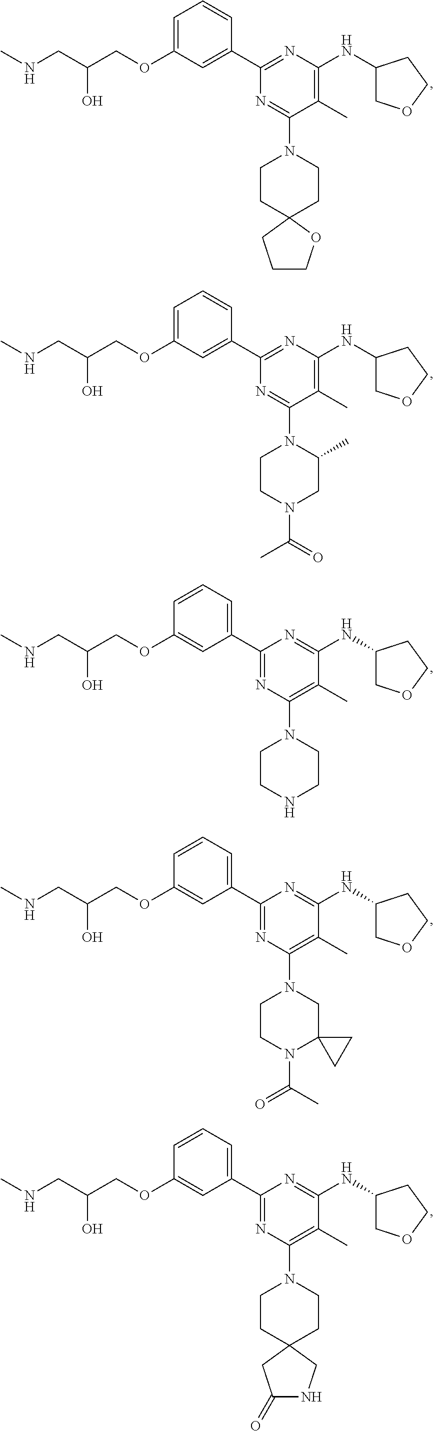 [SMILES: CNCC(O)COc1cccc(-c2nc(NC3CCOC3)c(C)c(N3CCC4(CCCO4)CC3)n2)c1 ; CNCC(O)COc1cccc(-c2nc(NC3CCOC3)c(C)c(N3CCN(C(C)=O)C[C@H]3C)n2)c1 ; CNCC(O)COc1cccc(-c2nc(N[C@@H]3CCOC3)c(C)c(N3CCNCC3)n2)c1 ; CNCC(O)COc1cccc(-c2nc(N[C@@H]3CCOC3)c(C)c(N3CCN(C(C)=O)C4(CC4)C3)n2)c1 ; CNCC(O)COc1cccc(-c2nc(N[C@@H]3CCOC3)c(C)c(N3CCC4(CC3)CNC(=O)C4)n2)c1]

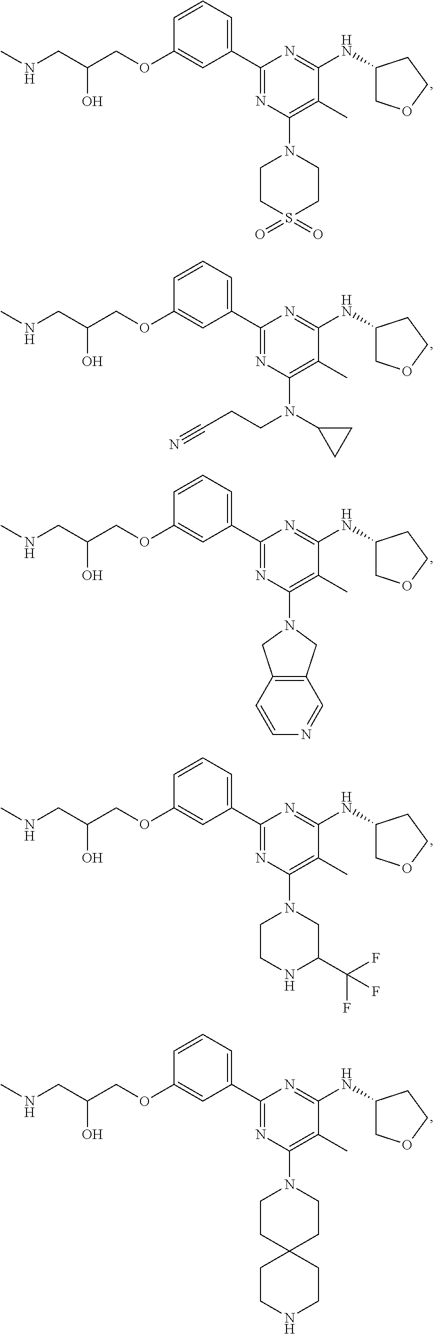

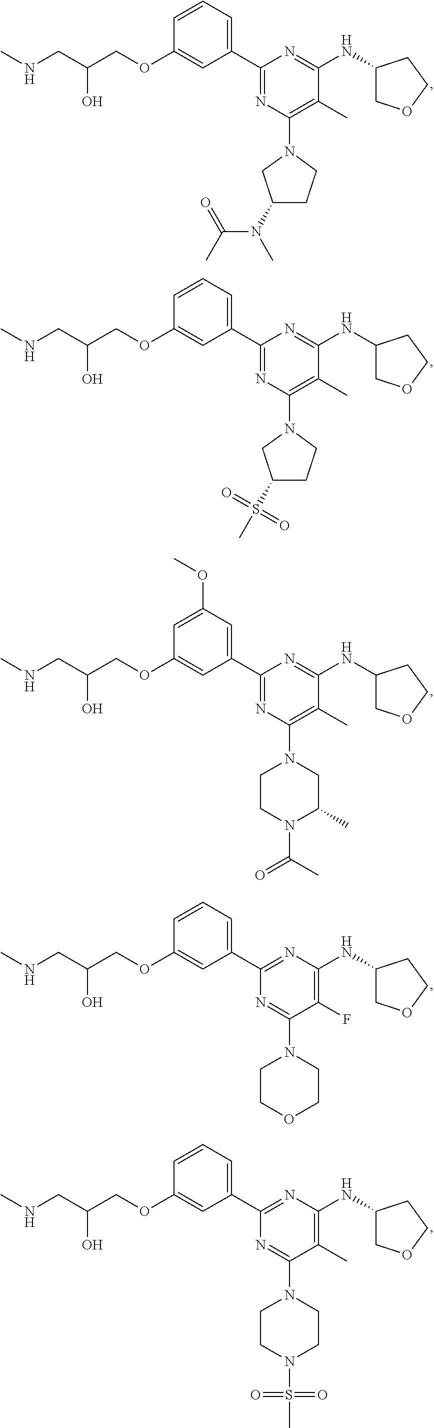

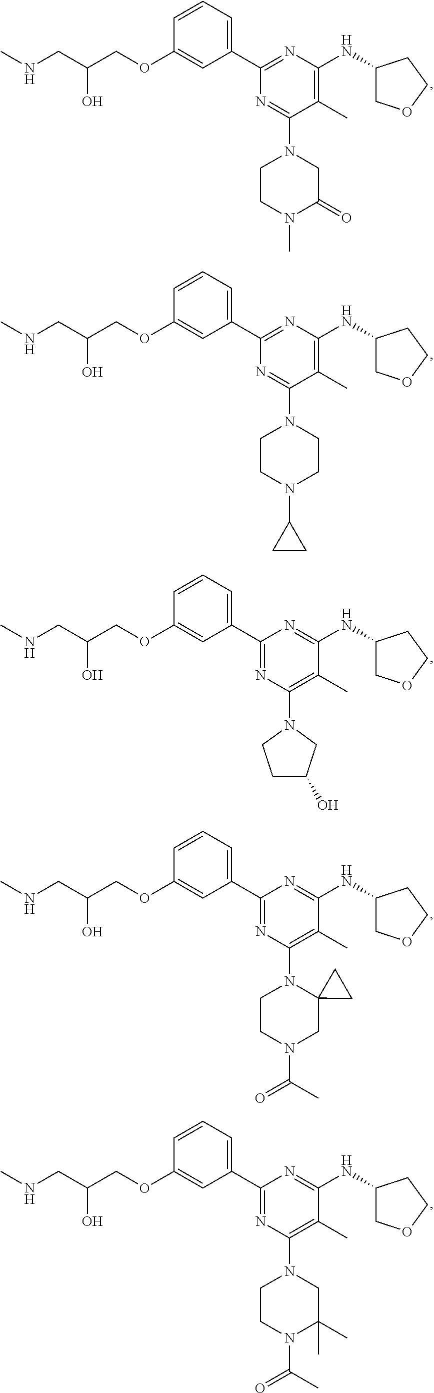 [SMILES: CNCC(O)COc1cccc(-c2nc(N[C@@H]3CCOC3)c(C)c(N3CCN(C)C(=O)C3)n2)c1 ; CNCC(O)COc1cccc(-c2nc(N[C@@H]3CCOC3)c(C)c(N3CCN(C4CC4)CC3)n2)c1 ; CNCC(O)COc1cccc(-c2nc(N[C@@H]3CCOC3)c(C)c(N3CC[C@@H](O)C3)n2)c1 ; CNCC(O)COc1cccc(-c2nc(N[C@@H]3CCOC3)c(C)c(N3CCN(C(C)=O)CC34CC4)n2)c1 ; CNCC(O)COc1cccc(-c2nc(N[C@@H]3CCOC3)c(C)c(N3CCN(C(C)=O)C(C)(C)C3)n2)c1]

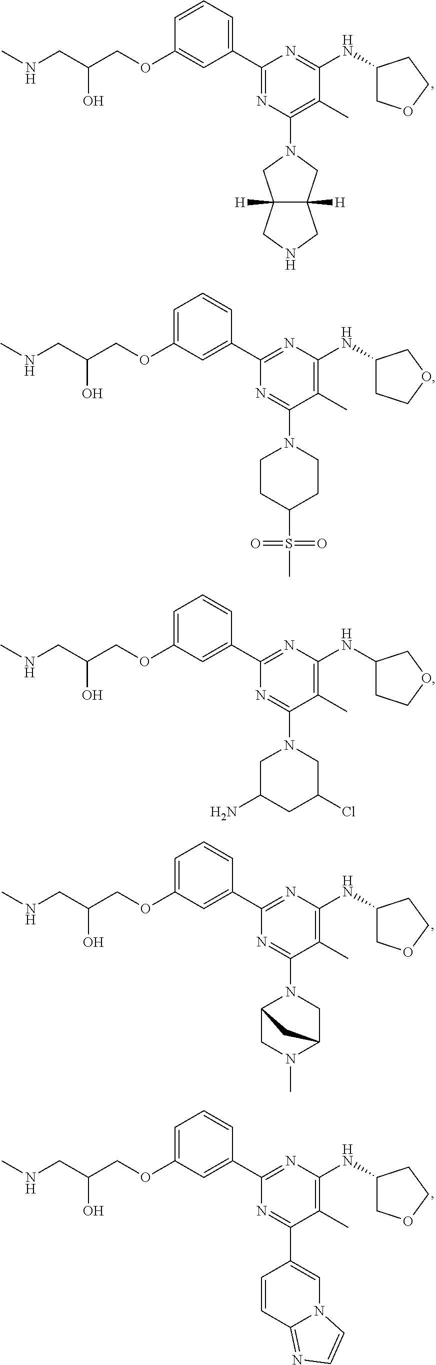

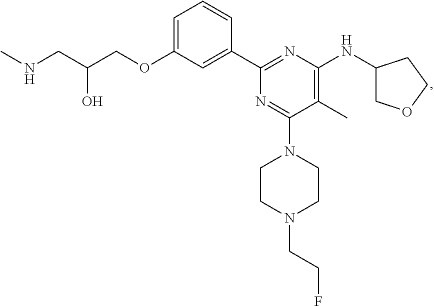 [SMILES: CNCC(O)COc1cccc(-c2nc(NC3CCOC3)c(C)c(N3CCN(CCF)CC3)n2)c1]

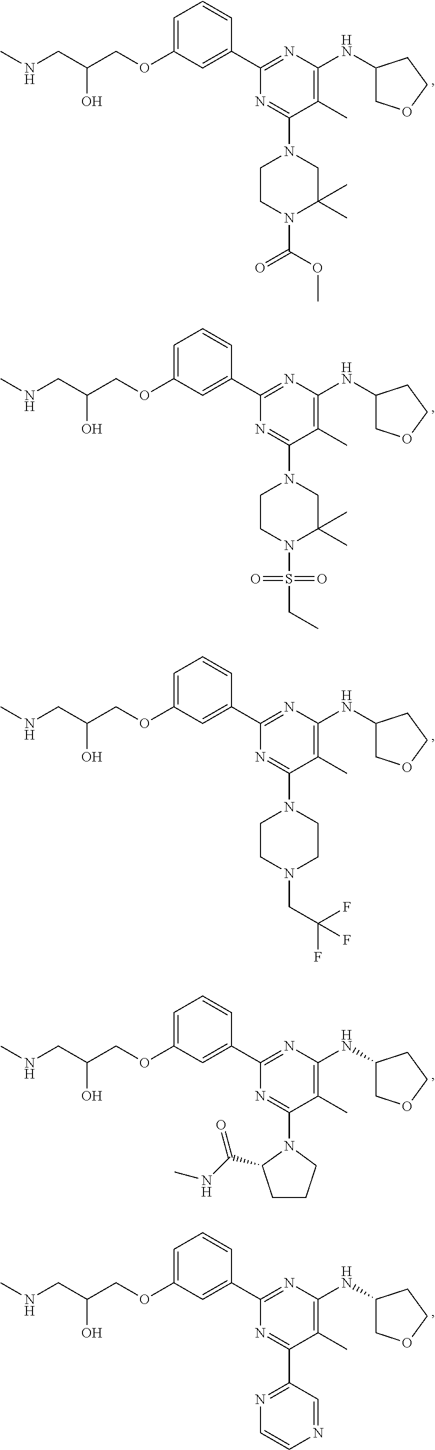 [SMILES: CNCC(O)COc1cccc(-c2nc(NC3CCOC3)c(C)c(N3CCN(C(=O)OC)C(C)(C)C3)n2)c1 ; CCS(=O)(=O)N1CCN(c2nc(-c3cccc(OCC(O)CNC)c3)nc(NC3CCOC3)c2C)CC1(C)C ; CNCC(O)COc1cccc(-c2nc(NC3CCOC3)c(C)c(N3CCN(CC(F)(F)F)CC3)n2)c1 ; CNCC(O)COc1cccc(-c2nc(N[C@@H]3CCOC3)c(C)c(N3CCC[C@@H]3C(=O)NC)n2)c1 ; CNCC(O)COc1cccc(-c2nc(N[C@@H]3CCOC3)c(C)c(-c3cnccn3)n2)c1]

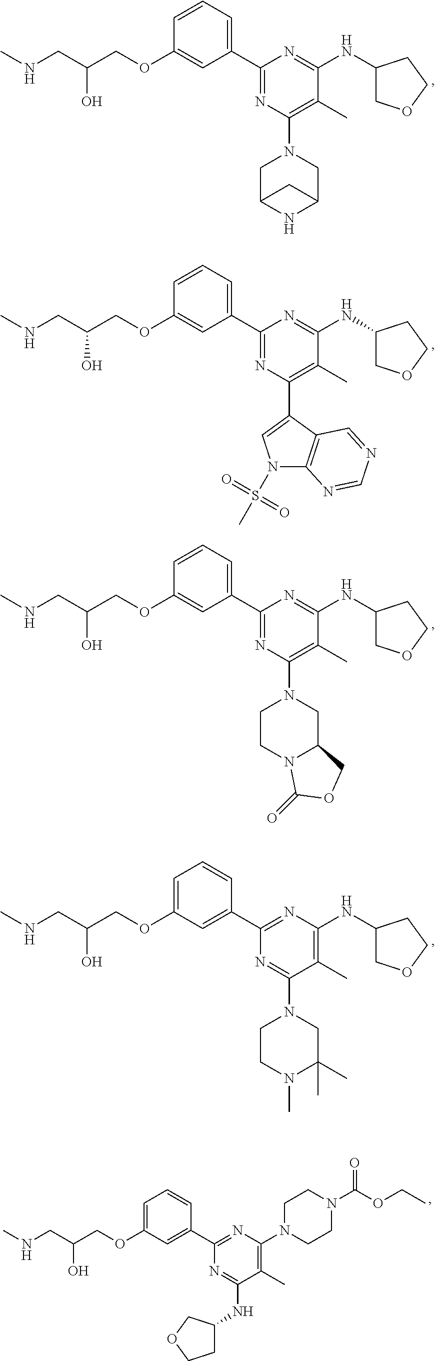

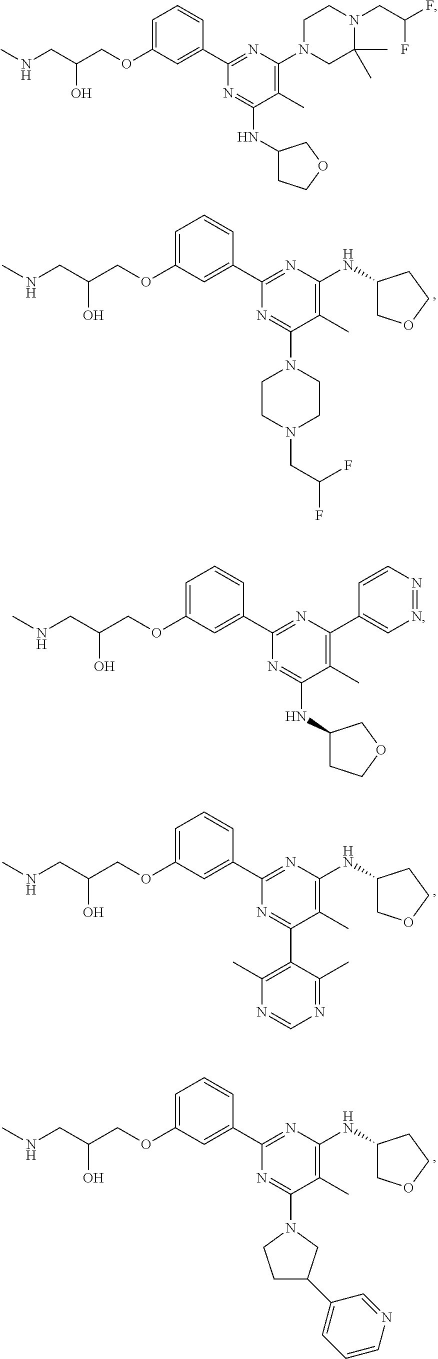 [SMILES: CNCC(O)COc1cccc(-c2nc(NC3CCOC3)c(C)c(N3CCN(CC(F)F)C(C)(C)C3)n2)c1 ; CNCC(O)COc1cccc(-c2nc(N[C@@H]3CCOC3)c(C)c(N3CCN(CC(F)F)CC3)n2)c1 ; CNCC(O)COc1cccc(-c2nc(N[C@@H]3CCOC3)c(C)c(-c3ccnnc3)n2)c1 ; CNCC(O)COc1cccc(-c2nc(N[C@@H]3CCOC3)c(C)c(-c3c(C)ncnc3C)n2)c1 ; CNCC(O)COc1cccc(-c2nc(N[C@@H]3CCOC3)c(C)c(N3CCC(c4cccnc4)C3)n2)c1]

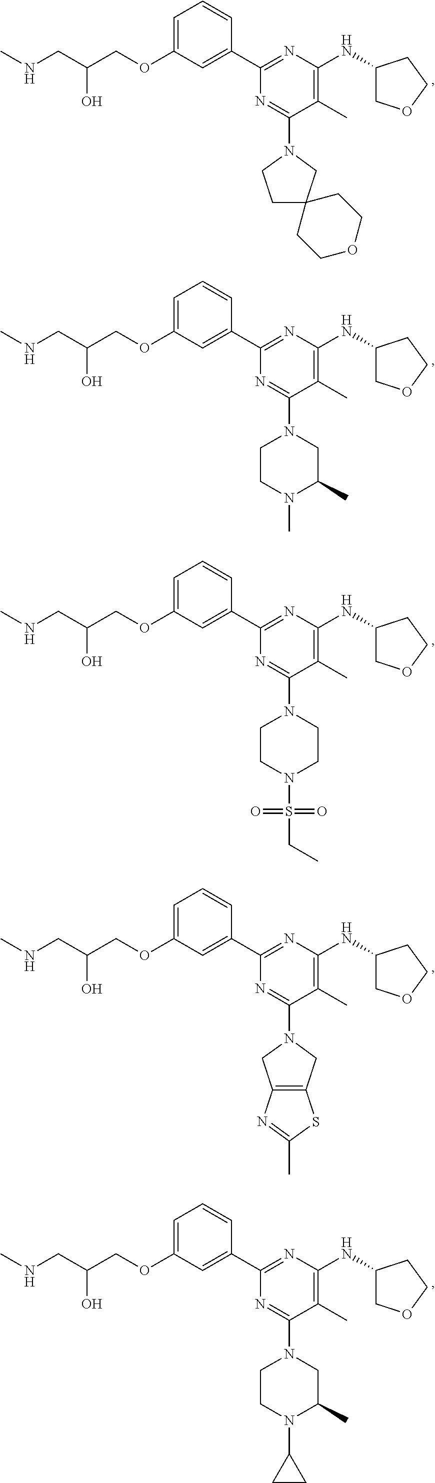 [SMILES: CNCC(O)COc1cccc(-c2nc(N[C@@H]3CCOC3)c(C)c(N3CCC4(CCOCC4)C3)n2)c1 ; CNCC(O)COc1cccc(-c2nc(N[C@@H]3CCOC3)c(C)c(N3CCN(C)[C@H](C)C3)n2)c1 ; CCS(=O)(=O)N1CCN(c2nc(-c3cccc(OCC(O)CNC)c3)nc(N[C@@H]3CCOC3)c2C)CC1 ; CNCC(O)COc1cccc(-c2nc(N[C@@H]3CCOC3)c(C)c(N3Cc4nc(C)sc4C3)n2)c1 ; CNCC(O)COc1cccc(-c2nc(N[C@@H]3CCOC3)c(C)c(N3CCN(C4CC4)[C@H](C)C3)n2)c1]

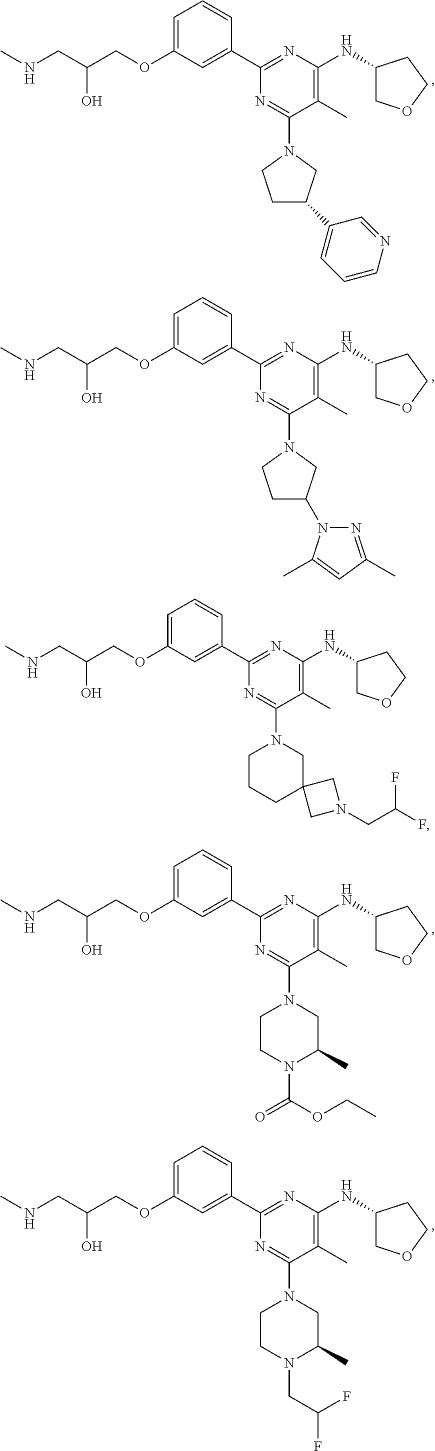 [SMILES: CNCC(O)COc1cccc(-c2nc(N[C@@H]3CCOC3)c(C)c(N3CC[C@@H](c4cccnc4)C3)n2)c1 ; CNCC(O)COc1cccc(-c2nc(N[C@@H]3CCOC3)c(C)c(N3CCC(n4nc(C)cc4C)C3)n2)c1 ; CNCC(O)COc1cccc(-c2nc(N[C@@H]3CCOC3)c(C)c(N3CCCC4(CN(CC(F)F)C4)C3)n2)c1 ; CCOC(=O)N1CCN(c2nc(-c3cccc(OCC(O)CNC)c3)nc(N[C@@H]3CCOC3)c2C)C[C@H]1C ; CNCC(O)COc1cccc(-c2nc(N[C@@H]3CCOC3)c(C)c(N3CCN(CC(F)F)[C@H](C)C3)n2)c1]

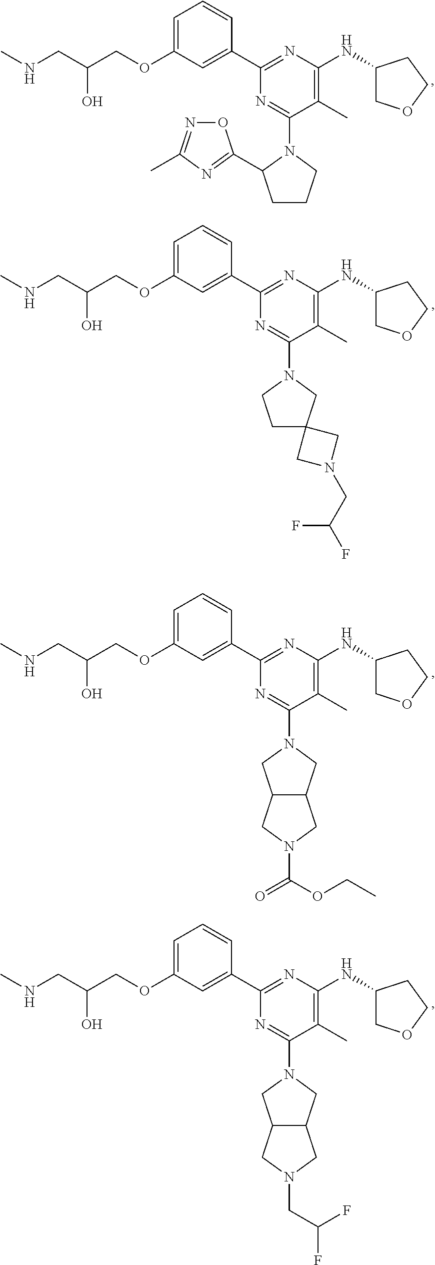 [SMILES: CNCC(O)COc1cccc(-c2nc(N[C@@H]3CCOC3)c(C)c(N3CCCC3c3nc(C)no3)n2)c1 ; CNCC(O)COc1cccc(-c2nc(N[C@@H]3CCOC3)c(C)c(N3CCC4(CN(CC(F)F)C4)C3)n2)c1 ; CCOC(=O)N1CC2CN(c3nc(-c4cccc(OCC(O)CNC)c4)nc(N[C@@H]4CCOC4)c3C)CC2C1 ; CNCC(O)COc1cccc(-c2nc(N[C@@H]3CCOC3)c(C)c(N3CC4CN(CC(F)F)CC4C3)n2)c1]

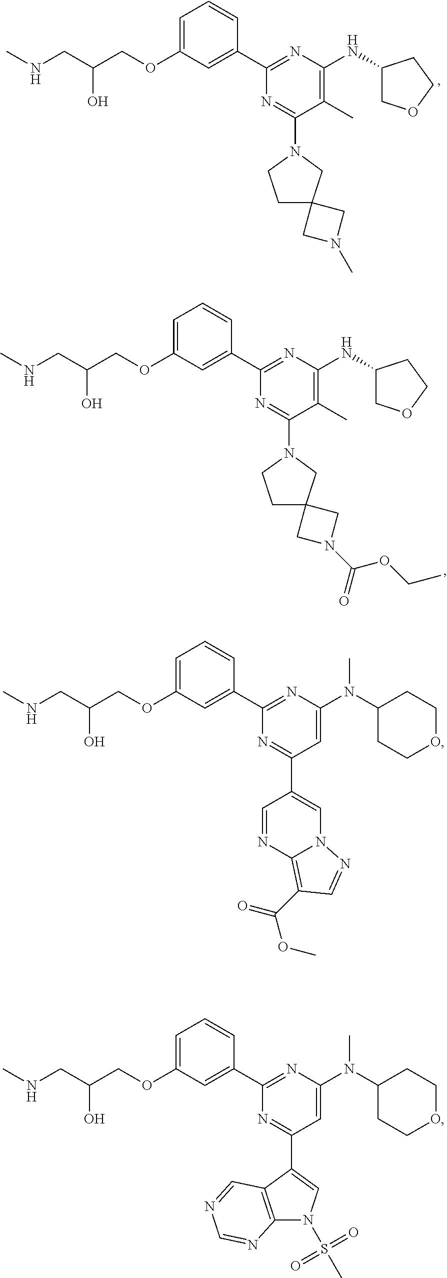

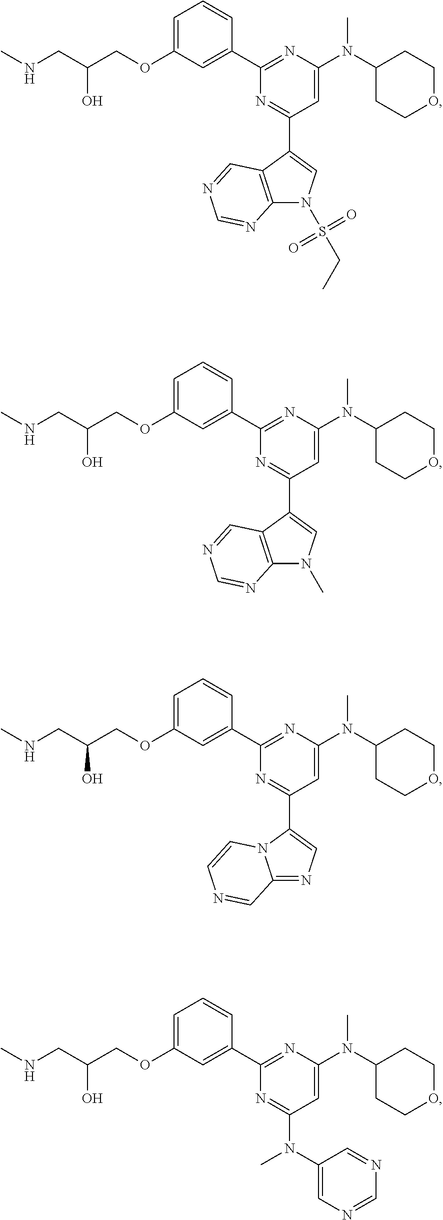 [SMILES: CCS(=O)(=O)n1cc(-c2cc(N(C)C3CCOCC3)nc(-c3cccc(OCC(O)CNC)c3)n2)c2cncnc21 ; CNCC(O)COc1cccc(-c2nc(-c3cn(C)c4ncncc34)cc(N(C)C3CCOCC3)n2)c1 ; CNC[C@H](O)COc1cccc(-c2nc(-c3cnc4cnccn34)cc(N(C)C3CCOCC3)n2)c1 ; CNCC(O)COc1cccc(-c2nc(N(C)c3cncnc3)cc(N(C)C3CCOCC3)n2)c1]

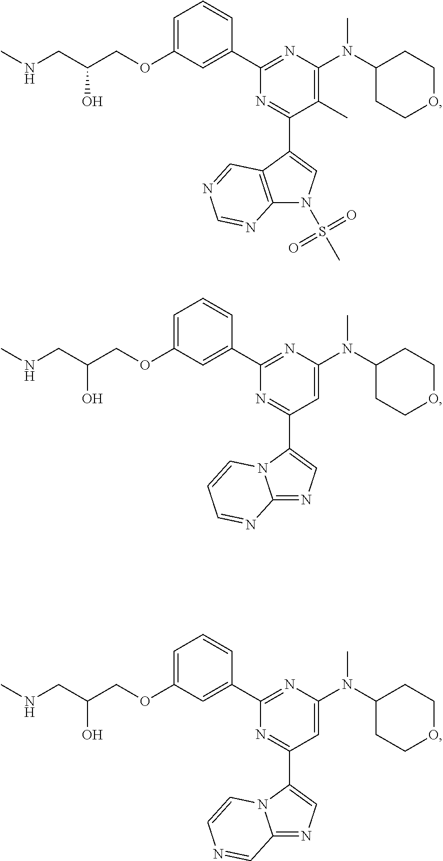 [SMILES: CNC[C@@H](O)COc1cccc(-c2nc(-c3cn(S(C)(=O)=O)c4ncncc34)c(C)c(N(C)C3CCOCC3)n2)c1 ; CNCC(O)COc1cccc(-c2nc(-c3cnc4ncccn34)cc(N(C)C3CCOCC3)n2)c1 ; CNCC(O)COc1cccc(-c2nc(-c3cnc4cnccn34)cc(N(C)C3CCOCC3)n2)c1]

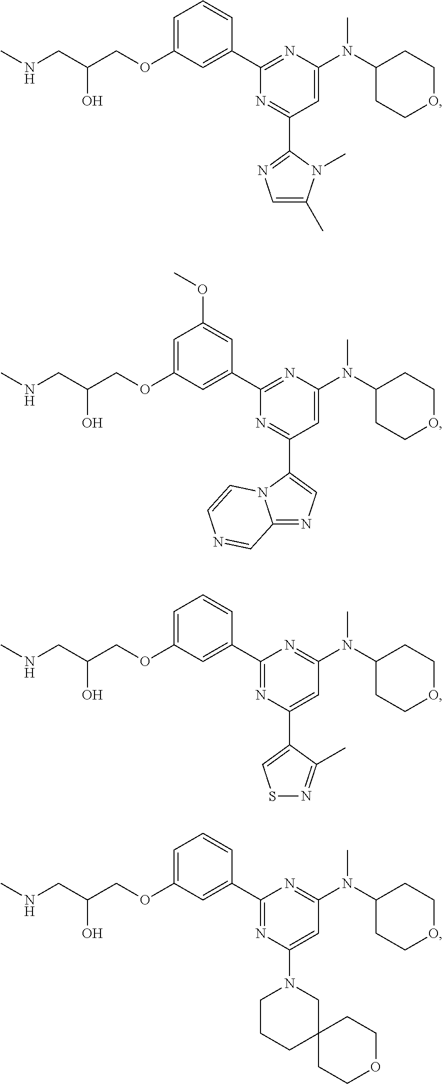 [SMILES: CNCC(O)COc1cccc(-c2nc(-c3ncc(C)n3C)cc(N(C)C3CCOCC3)n2)c1 ; CNCC(O)COc1cc(OC)cc(-c2nc(-c3cnc4cnccn34)cc(N(C)C3CCOCC3)n2)c1 ; CNCC(O)COc1cccc(-c2nc(-c3csnc3C)cc(N(C)C3CCOCC3)n2)c1 ; CNCC(O)COc1cccc(-c2nc(N3CCCC4(CCOCC4)C3)cc(N(C)C3CCOCC3)n2)c1]

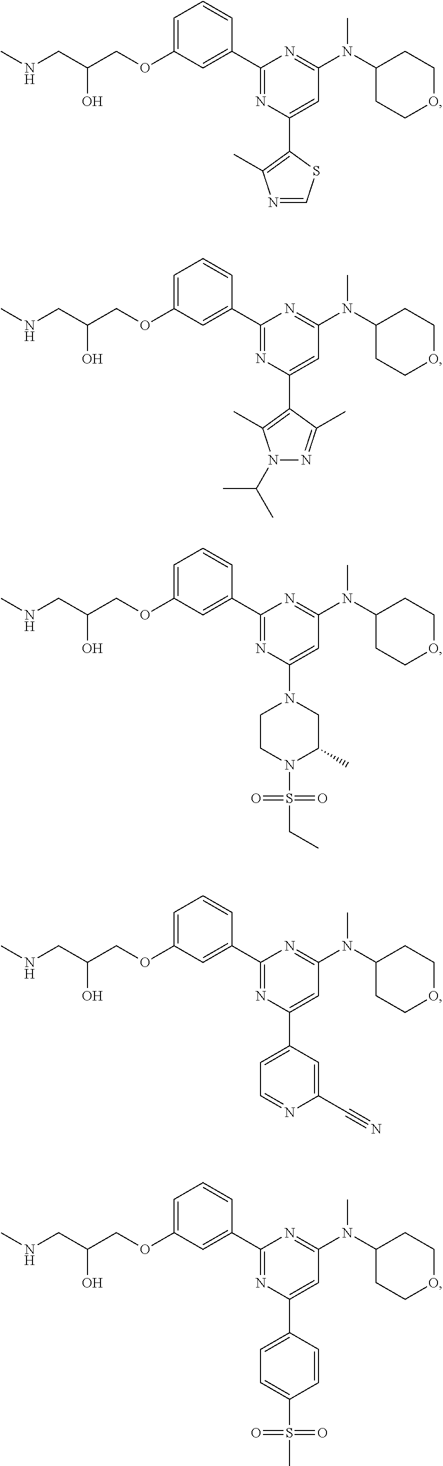 [SMILES: CNCC(O)COc1cccc(-c2nc(-c3scnc3C)cc(N(C)C3CCOCC3)n2)c1 ; CNCC(O)COc1cccc(-c2nc(-c3c(C)nn(C(C)C)c3C)cc(N(C)C3CCOCC3)n2)c1 ; CCS(=O)(=O)N1CCN(c2cc(N(C)C3CCOCC3)nc(-c3cccc(OCC(O)CNC)c3)n2)C[C@@H]1C ; CNCC(O)COc1cccc(-c2nc(-c3ccnc(C#N)c3)cc(N(C)C3CCOCC3)n2)c1 ; CNCC(O)COc1cccc(-c2nc(-c3ccc(S(C)(=O)=O)cc3)cc(N(C)C3CCOCC3)n2)c1]

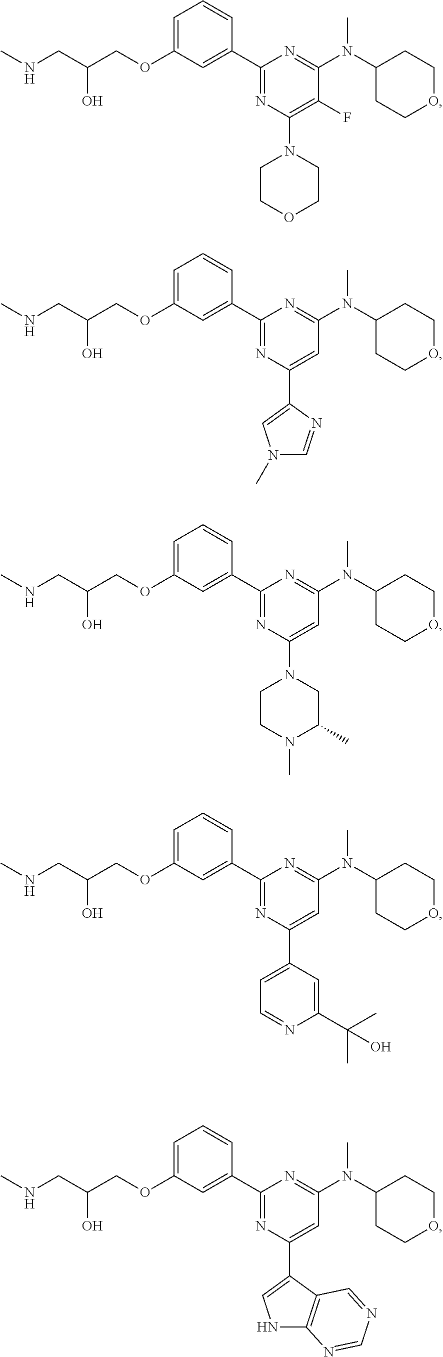 [SMILES: CNCC(O)COc1cccc(-c2nc(N3CCOCC3)c(F)c(N(C)C3CCOCC3)n2)c1 ; CNCC(O)COc1cccc(-c2nc(-c3cn(C)cn3)cc(N(C)C3CCOCC3)n2)c1 ; CNCC(O)COc1cccc(-c2nc(N3CCN(C)[C@@H](C)C3)cc(N(C)C3CCOCC3)n2)c1 ; CNCC(O)COc1cccc(-c2nc(-c3ccnc(C(C)(C)O)c3)cc(N(C)C3CCOCC3)n2)c1 ; CNCC(O)COc1cccc(-c2nc(-c3c[nH]c4ncncc34)cc(N(C)C3CCOCC3)n2)c1]

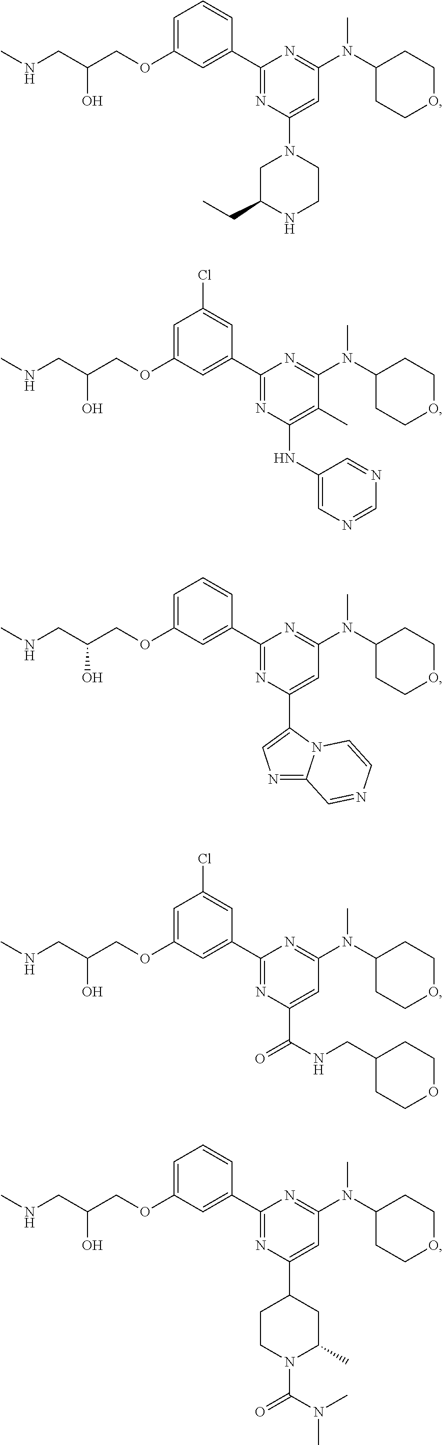 [SMILES: CC[C@H]1CN(c2cc(N(C)C3CCOCC3)nc(-c3cccc(OCC(O)CNC)c3)n2)CCN1 ; CNCC(O)COc1cc(Cl)cc(-c2nc(Nc3cncnc3)c(C)c(N(C)C3CCOCC3)n2)c1 ; CNC[C@@H](O)COc1cccc(-c2nc(-c3cnc4cnccn34)cc(N(C)C3CCOCC3)n2)c1 ; CNCC(O)COc1cc(Cl)cc(-c2nc(C(=O)NCC3CCOCC3)cc(N(C)C3CCOCC3)n2)c1 ; CNCC(O)COc1cccc(-c2nc(C3CCN(C(=O)N(C)C)[C@@H](C)C3)cc(N(C)C3CCOCC3)n2)c1]

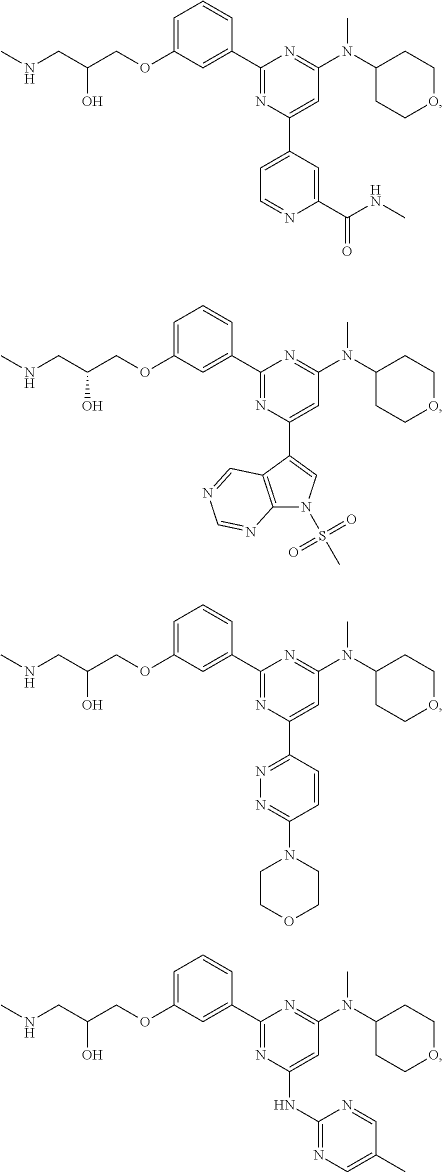 [SMILES: CNCC(O)COc1cccc(-c2nc(-c3ccnc(C(=O)NC)c3)cc(N(C)C3CCOCC3)n2)c1 ; CNC[C@@H](O)COc1cccc(-c2nc(-c3cn(S(C)(=O)=O)c4ncncc34)cc(N(C)C3CCOCC3)n2)c1 ; CNCC(O)COc1cccc(-c2nc(-c3ccc(N4CCOCC4)nn3)cc(N(C)C3CCOCC3)n2)c1 ; CNCC(O)COc1cccc(-c2nc(Nc3ncc(C)cn3)cc(N(C)C3CCOCC3)n2)c1]

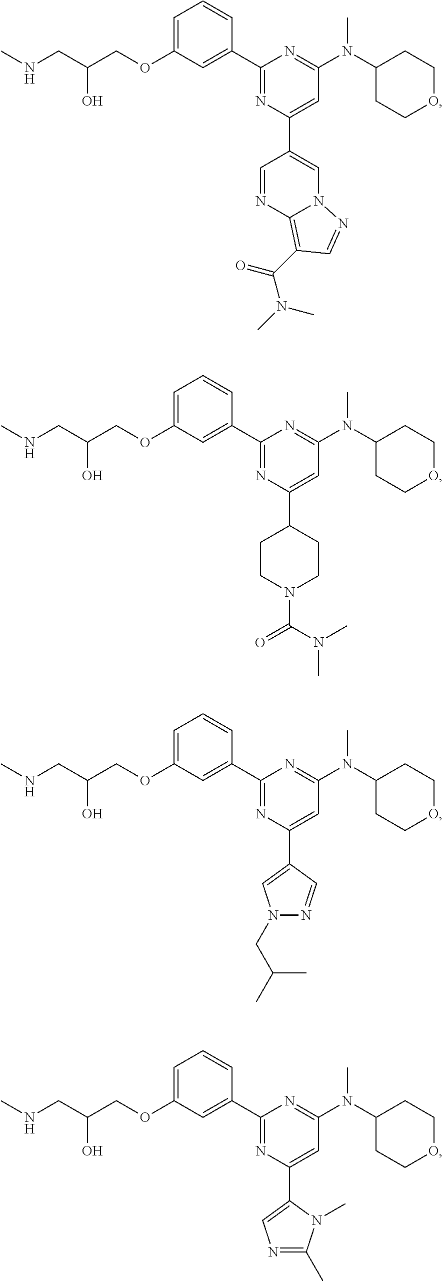 [SMILES: CNCC(O)COc1cccc(-c2nc(-c3cnc4c(C(=O)N(C)C)cnn4c3)cc(N(C)C3CCOCC3)n2)c1 ; CNCC(O)COc1cccc(-c2nc(C3CCN(C(=O)N(C)C)CC3)cc(N(C)C3CCOCC3)n2)c1 ; CNCC(O)COc1cccc(-c2nc(-c3cnn(CC(C)C)c3)cc(N(C)C3CCOCC3)n2)c1 ; CNCC(O)COc1cccc(-c2nc(-c3cnc(C)n3C)cc(N(C)C3CCOCC3)n2)c1]

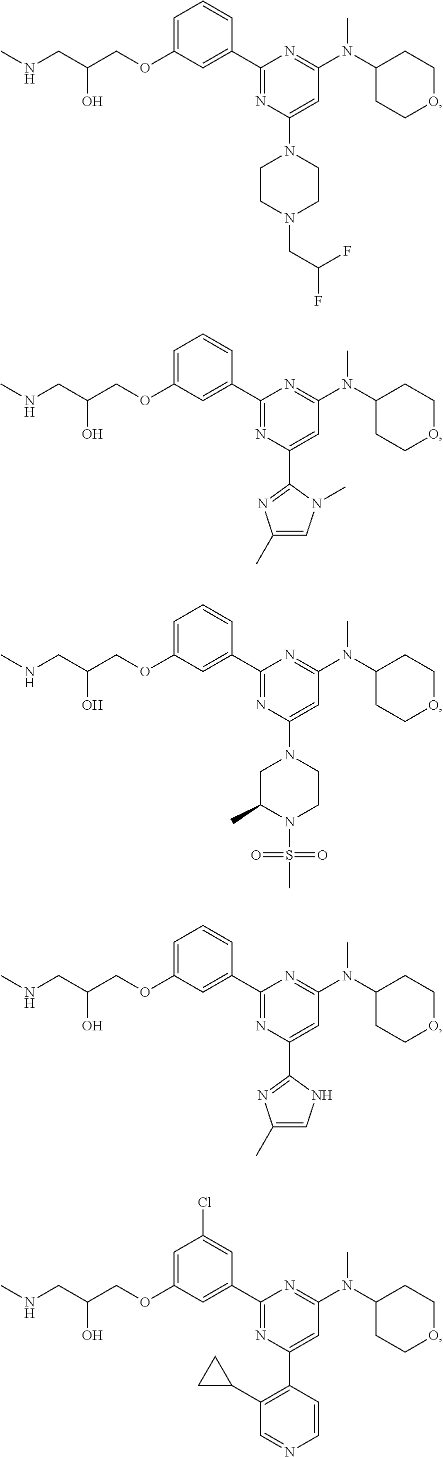 [SMILES: CNCC(O)COc1cccc(-c2nc(N3CCN(CC(F)F)CC3)cc(N(C)C3CCOCC3)n2)c1 ; CNCC(O)COc1cccc(-c2nc(-c3nc(C)cn3C)cc(N(C)C3CCOCC3)n2)c1 ; CNCC(O)COc1cccc(-c2nc(N3CCN(S(C)(=O)=O)[C@@H](C)C3)cc(N(C)C3CCOCC3)n2)c1 ; CNCC(O)COc1cccc(-c2nc(-c3nc(C)c[nH]3)cc(N(C)C3CCOCC3)n2)c1 ; CNCC(O)COc1cc(Cl)cc(-c2nc(-c3ccncc3C3CC3)cc(N(C)C3CCOCC3)n2)c1]

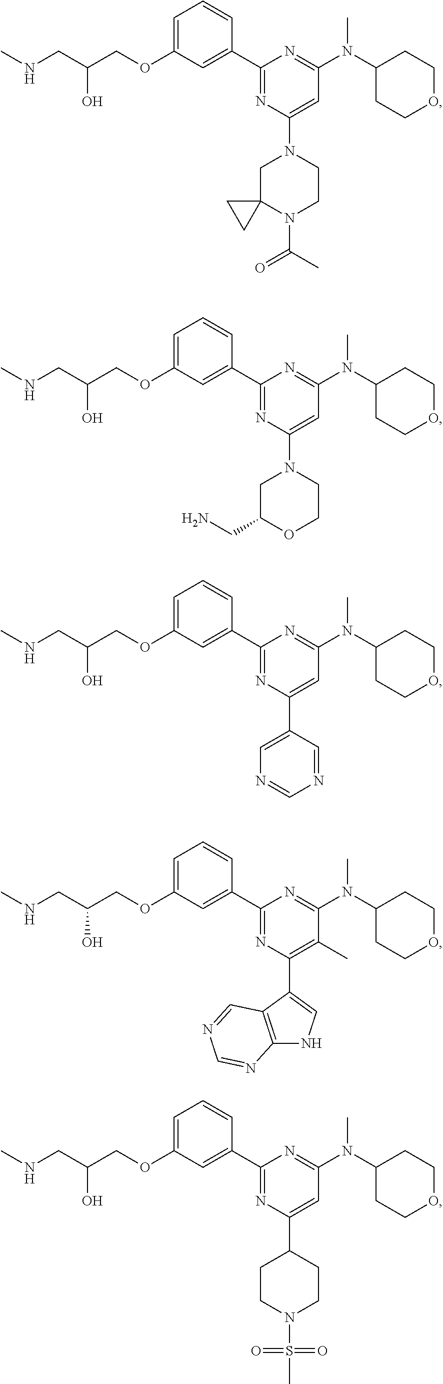

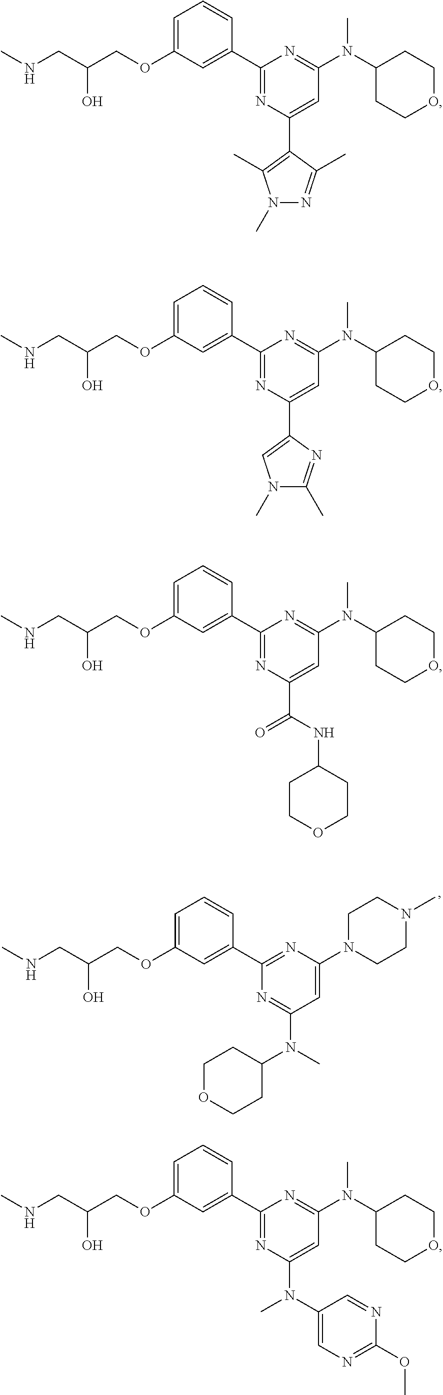

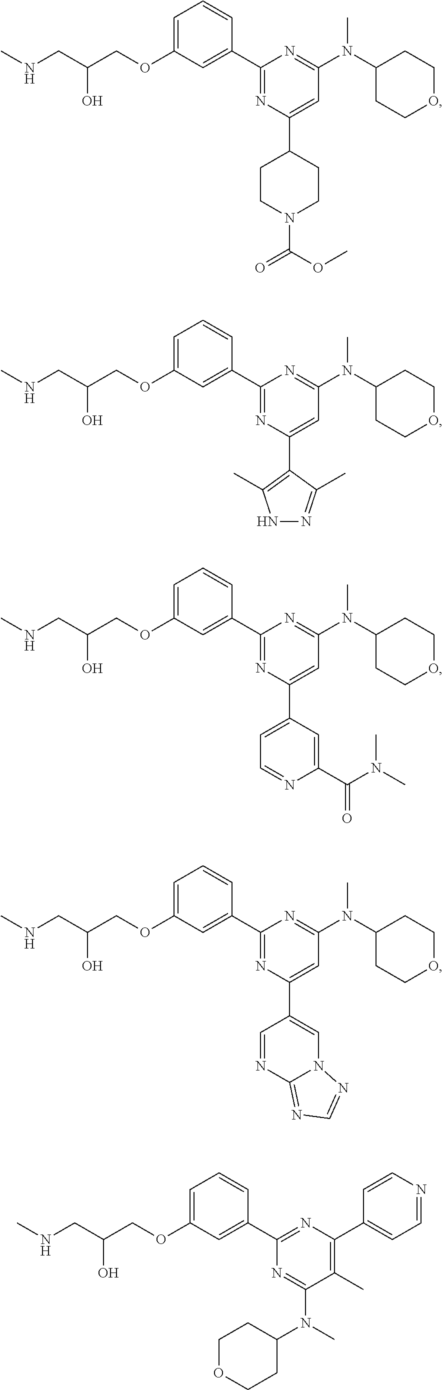 [SMILES: CNCC(O)COc1cccc(-c2nc(C3CCN(C(=O)OC)CC3)cc(N(C)C3CCOCC3)n2)c1 ; CNCC(O)COc1cccc(-c2nc(-c3c(C)n[nH]c3C)cc(N(C)C3CCOCC3)n2)c1 ; CNCC(O)COc1cccc(-c2nc(-c3ccnc(C(=O)N(C)C)c3)cc(N(C)C3CCOCC3)n2)c1 ; CNCC(O)COc1cccc(-c2nc(-c3cnc4ncnn4c3)cc(N(C)C3CCOCC3)n2)c1 ; CNCC(O)COc1cccc(-c2nc(-c3ccncc3)c(C)c(N(C)C3CCOCC3)n2)c1]

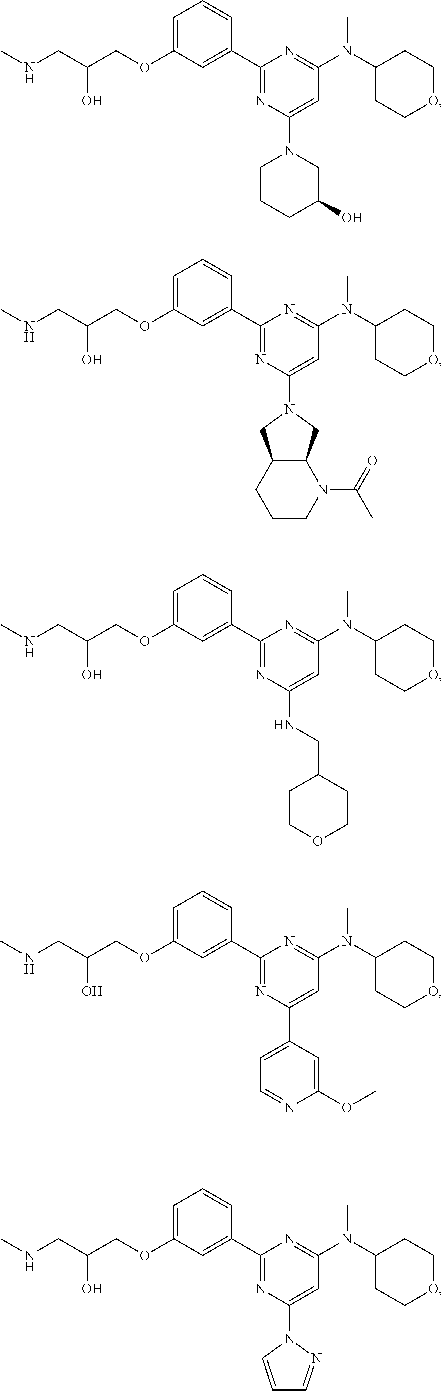 [SMILES: CNCC(O)COc1cccc(-c2nc(N3CCC[C@H](O)C3)cc(N(C)C3CCOCC3)n2)c1 ; CNCC(O)COc1cccc(-c2nc(N3C[C@@H]4CCCN(C(C)=O)[C@@H]4C3)cc(N(C)C3CCOCC3)n2)c1 ; CNCC(O)COc1cccc(-c2nc(NCC3CCOCC3)cc(N(C)C3CCOCC3)n2)c1 ; CNCC(O)COc1cccc(-c2nc(-c3ccnc(OC)c3)cc(N(C)C3CCOCC3)n2)c1 ; CNCC(O)COc1cccc(-c2nc(N(C)C3CCOCC3)cc(-n3cccn3)n2)c1]

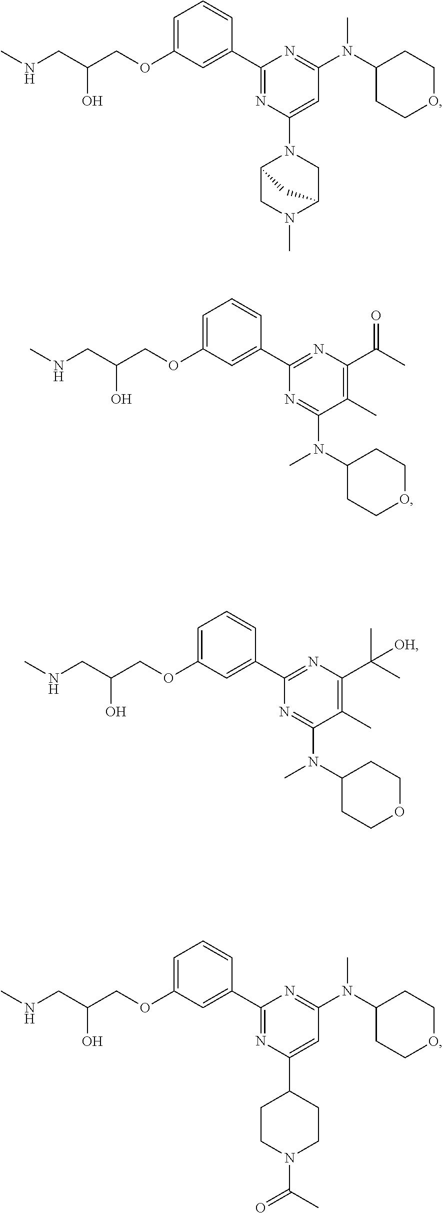 [SMILES: CNCC(O)COc1cccc(-c2nc(N(C)C3CCOCC3)cc(N3C[C@@H]4C[C@H]3CN4C)n2)c1 ; CNCC(O)COc1cccc(-c2nc(C(C)=O)c(C)c(N(C)C3CCOCC3)n2)c1 ; CNCC(O)COc1cccc(-c2nc(N(C)C3CCOCC3)c(C)c(C(C)(C)O)n2)c1 ; CNCC(O)COc1cccc(-c2nc(C3CCN(C(C)=O)CC3)cc(N(C)C3CCOCC3)n2)c1]

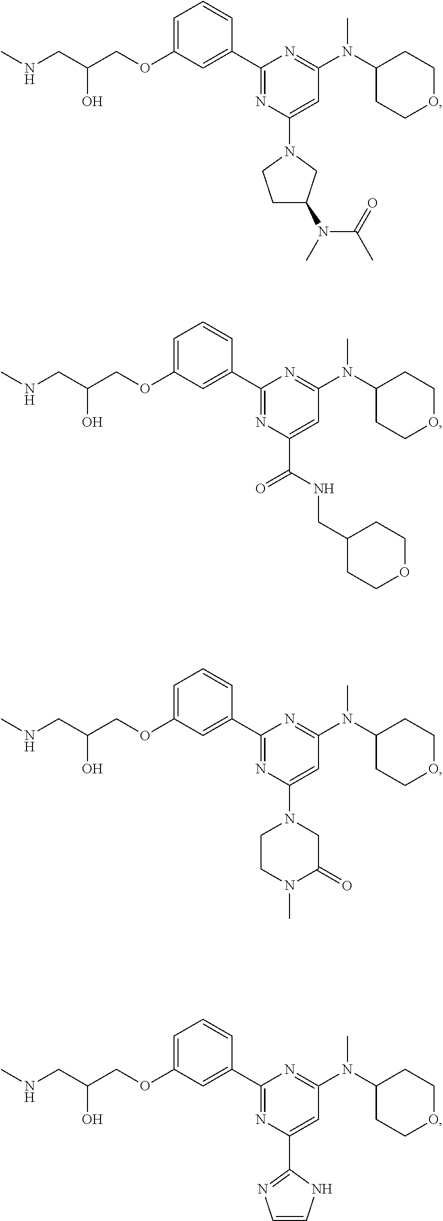 [SMILES: CNCC(O)COc1cccc(-c2nc(N3CC[C@H](N(C)C(C)=O)C3)cc(N(C)C3CCOCC3)n2)c1 ; CNCC(O)COc1cccc(-c2nc(C(=O)NCC3CCOCC3)cc(N(C)C3CCOCC3)n2)c1 ; CNCC(O)COc1cccc(-c2nc(N3CCN(C)C(=O)C3)cc(N(C)C3CCOCC3)n2)c1 ; CNCC(O)COc1cccc(-c2nc(-c3ncc[nH]3)cc(N(C)C3CCOCC3)n2)c1]

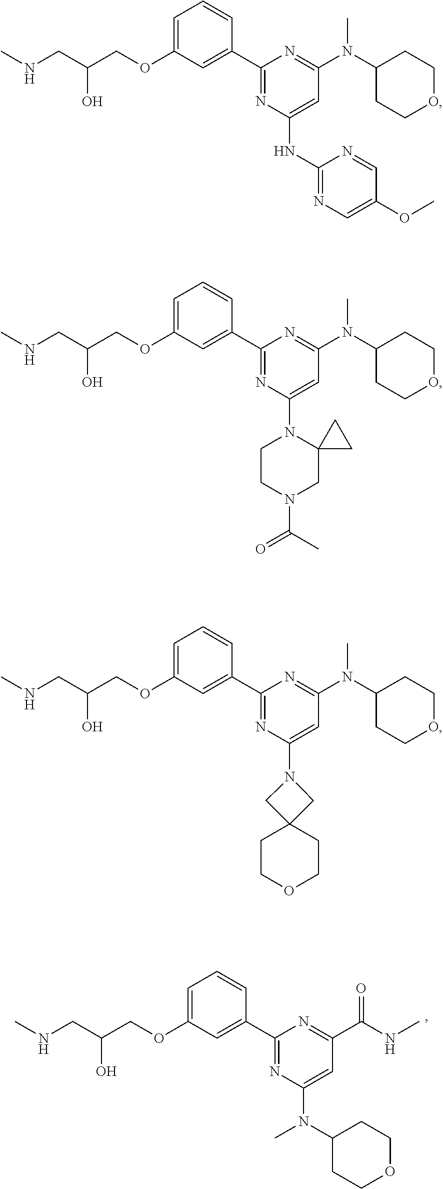

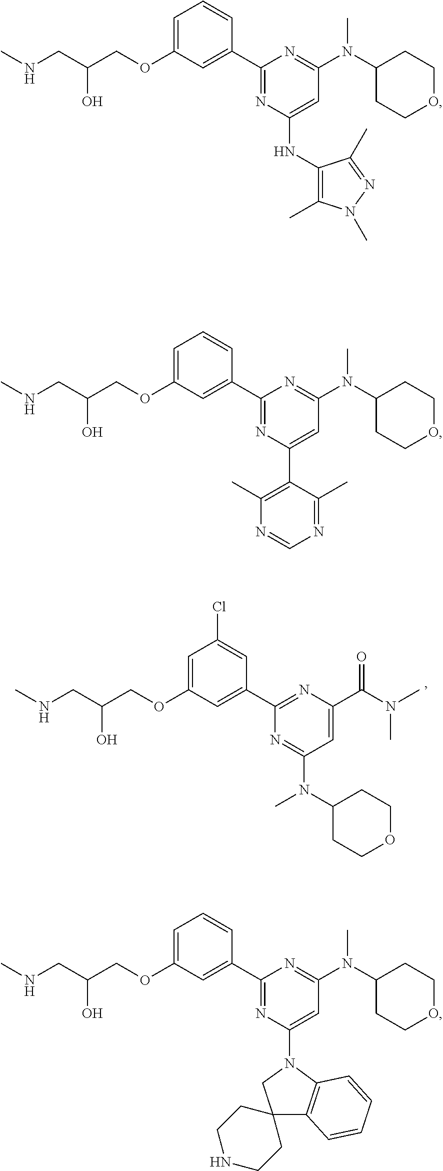

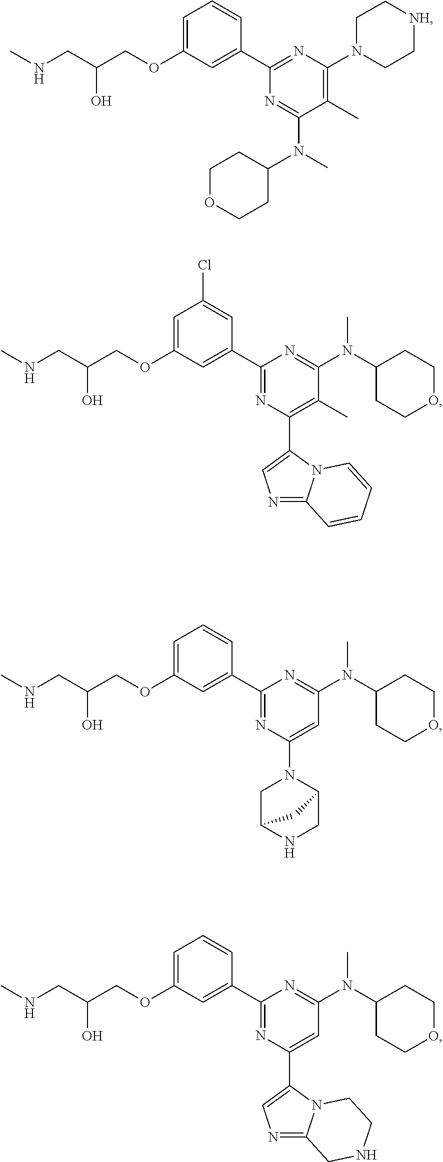 [SMILES: CNCC(O)COc1cccc(-c2nc(N3CCNCC3)c(C)c(N(C)C3CCOCC3)n2)c1 ; CNCC(O)COc1cc(Cl)cc(-c2nc(-c3cnc4ccccn34)c(C)c(N(C)C3CCOCC3)n2)c1 ; CNCC(O)COc1cccc(-c2nc(N(C)C3CCOCC3)cc(N3C[C@H]4C[C@@H]3CN4)n2)c1 ; CNCC(O)COc1cccc(-c2nc(-c3cnc4n3CCNC4)cc(N(C)C3CCOCC3)n2)c1]

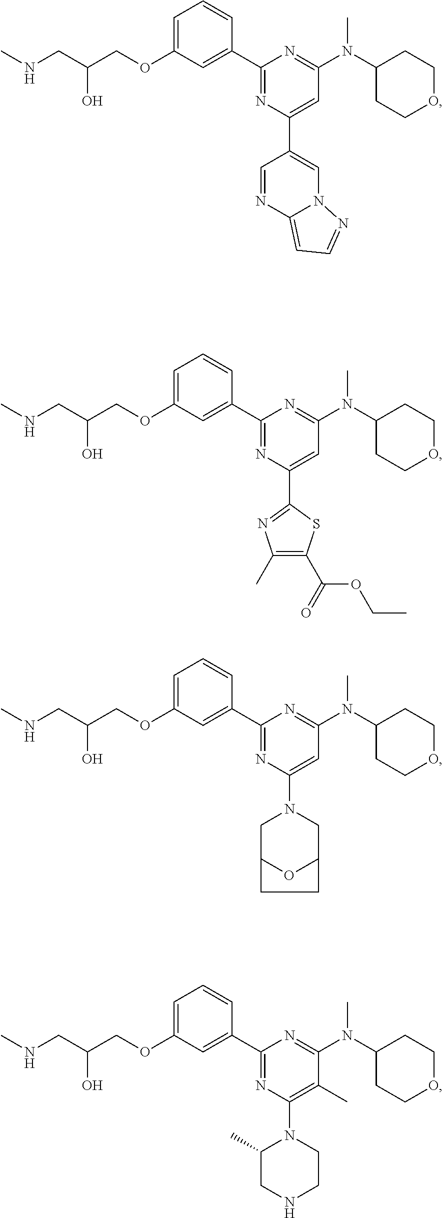 [SMILES: CNCC(O)COc1cccc(-c2nc(-c3cnc4ccnn4c3)cc(N(C)C3CCOCC3)n2)c1 ; CCOC(=O)c1sc(-c2cc(N(C)C3CCOCC3)nc(-c3cccc(OCC(O)CNC)c3)n2)nc1C ; CNCC(O)COc1cccc(-c2nc(N3CC4CCC(C3)O4)cc(N(C)C3CCOCC3)n2)c1 ; CNCC(O)COc1cccc(-c2nc(N(C)C3CCOCC3)c(C)c(N3CCNC[C@@H]3C)n2)c1]

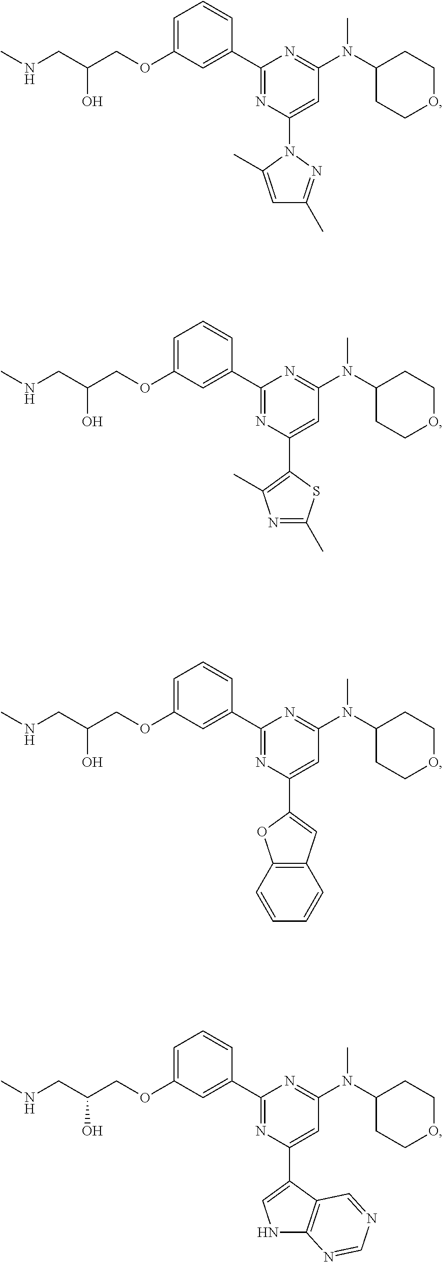 [SMILES: CNCC(O)COc1cccc(-c2nc(N(C)C3CCOCC3)cc(-n3nc(C)cc3C)n2)c1 ; CNCC(O)COc1cccc(-c2nc(-c3sc(C)nc3C)cc(N(C)C3CCOCC3)n2)c1 ; CNCC(O)COc1cccc(-c2nc(-c3cc4ccccc4o3)cc(N(C)C3CCOCC3)n2)c1 ; CNC[C@@H](O)COc1cccc(-c2nc(-c3c[nH]c4ncncc34)cc(N(C)C3CCOCC3)n2)c1]

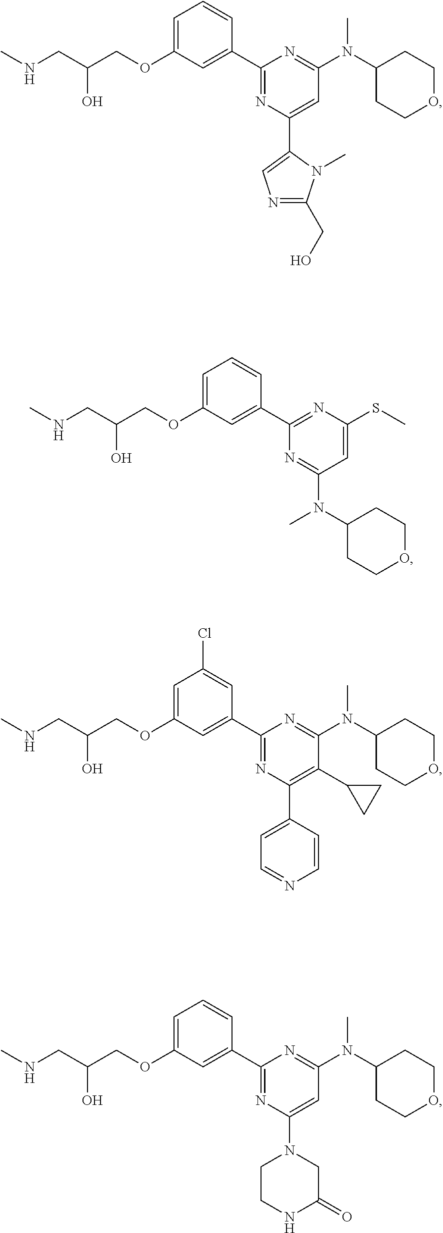 [SMILES: CNCC(O)COc1cccc(-c2nc(-c3cnc(CO)n3C)cc(N(C)C3CCOCC3)n2)c1 ; CNCC(O)COc1cccc(-c2nc(SC)cc(N(C)C3CCOCC3)n2)c1 ; CNCC(O)COc1cc(Cl)cc(-c2nc(-c3ccncc3)c(C3CC3)c(N(C)C3CCOCC3)n2)c1 ; CNCC(O)COc1cccc(-c2nc(N3CCNC(=O)C3)cc(N(C)C3CCOCC3)n2)c1]

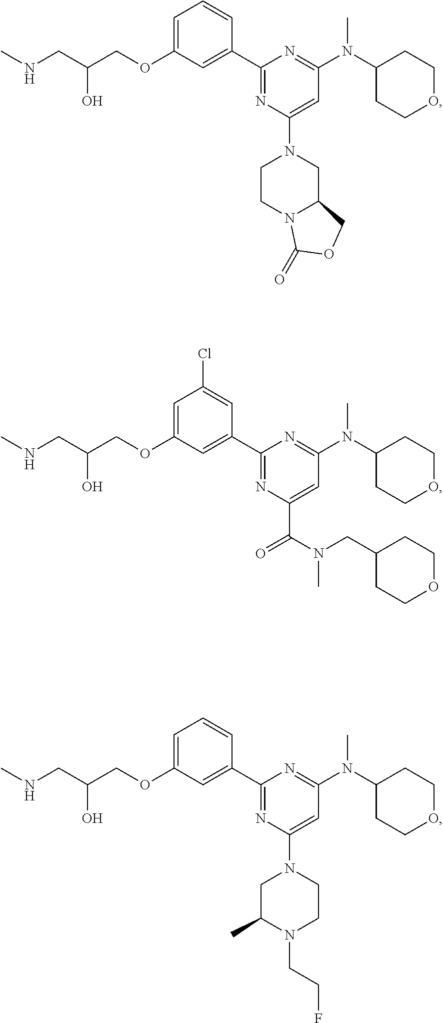 [SMILES: CNCC(O)COc1cccc(-c2nc(N3CCN4C(=O)OC[C@@H]4C3)cc(N(C)C3CCOCC3)n2)c1 ; CNCC(O)COc1cc(Cl)cc(-c2nc(C(=O)N(C)CC3CCOCC3)cc(N(C)C3CCOCC3)n2)c1 ; CNCC(O)COc1cccc(-c2nc(N3CCN(CCF)[C@@H](C)C3)cc(N(C)C3CCOCC3)n2)c1]

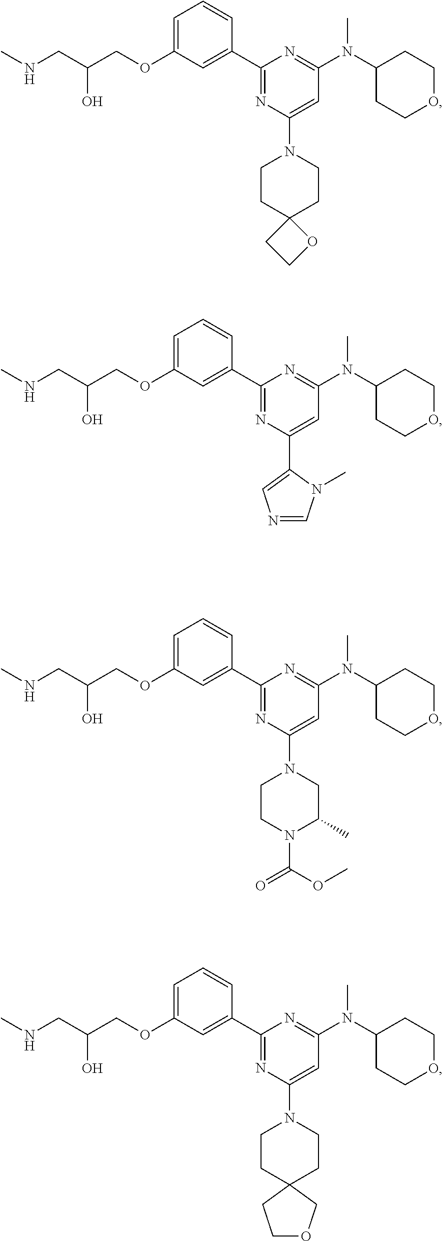 [SMILES: CNCC(O)COc1cccc(-c2nc(N3CCC4(CCO4)CC3)cc(N(C)C3CCOCC3)n2)c1 ; CNCC(O)COc1cccc(-c2nc(-c3cncn3C)cc(N(C)C3CCOCC3)n2)c1 ; CNCC(O)COc1cccc(-c2nc(N3CCN(C(=O)OC)[C@@H](C)C3)cc(N(C)C3CCOCC3)n2)c1 ; CNCC(O)COc1cccc(-c2nc(N3CCC4(CCOC4)CC3)cc(N(C)C3CCOCC3)n2)c1]

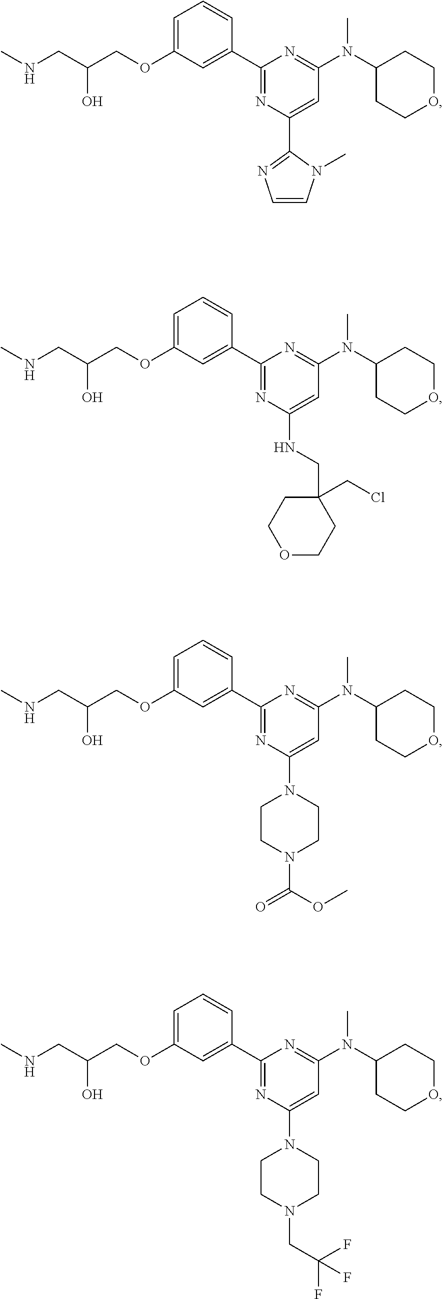 [SMILES: CNCC(O)COc1cccc(-c2nc(-c3nccn3C)cc(N(C)C3CCOCC3)n2)c1 ; CNCC(O)COc1cccc(-c2nc(NCC3(CCl)CCOCC3)cc(N(C)C3CCOCC3)n2)c1 ; CNCC(O)COc1cccc(-c2nc(N3CCN(C(=O)OC)CC3)cc(N(C)C3CCOCC3)n2)c1 ; CNCC(O)COc1cccc(-c2nc(N3CCN(CC(F)(F)F)CC3)cc(N(C)C3CCOCC3)n2)c1]

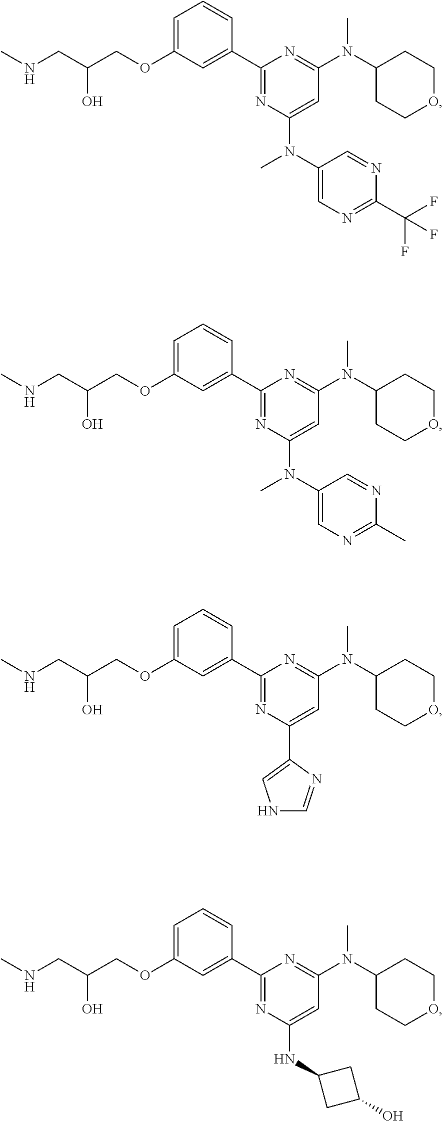 [SMILES: CNCC(O)COc1cccc(-c2nc(N(C)c3cnc(C(F)(F)F)nc3)cc(N(C)C3CCOCC3)n2)c1 ; CNCC(O)COc1cccc(-c2nc(N(C)c3cnc(C)nc3)cc(N(C)C3CCOCC3)n2)c1 ; CNCC(O)COc1cccc(-c2nc(-c3c[nH]cn3)cc(N(C)C3CCOCC3)n2)c1 ; CNCC(O)COc1cccc(-c2nc(N[C@H]3C[C@H](O)C3)cc(N(C)C3CCOCC3)n2)c1]

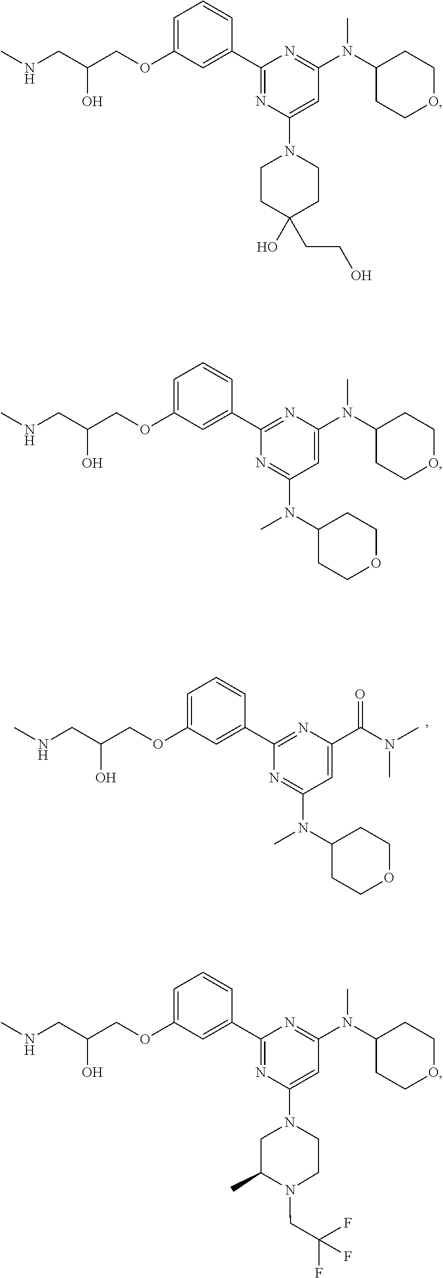

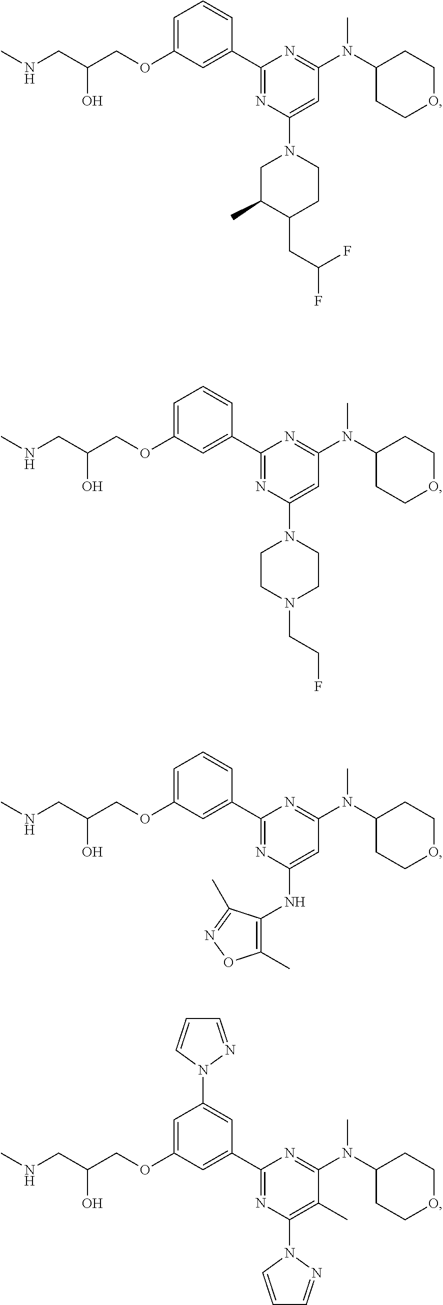 [SMILES: CNCC(O)COc1cccc(-c2nc(N3CCC(CC(F)F)[C@@H](C)C3)cc(N(C)C3CCOCC3)n2)c1 ; CNCC(O)COc1cccc(-c2nc(N3CCN(CCF)CC3)cc(N(C)C3CCOCC3)n2)c1 ; CNCC(O)COc1cccc(-c2nc(Nc3c(C)noc3C)cc(N(C)C3CCOCC3)n2)c1 ; CNCC(O)COc1cc(-c2nc(N(C)C3CCOCC3)c(C)c(-n3cccn3)n2)cc(-n2cccn2)c1]

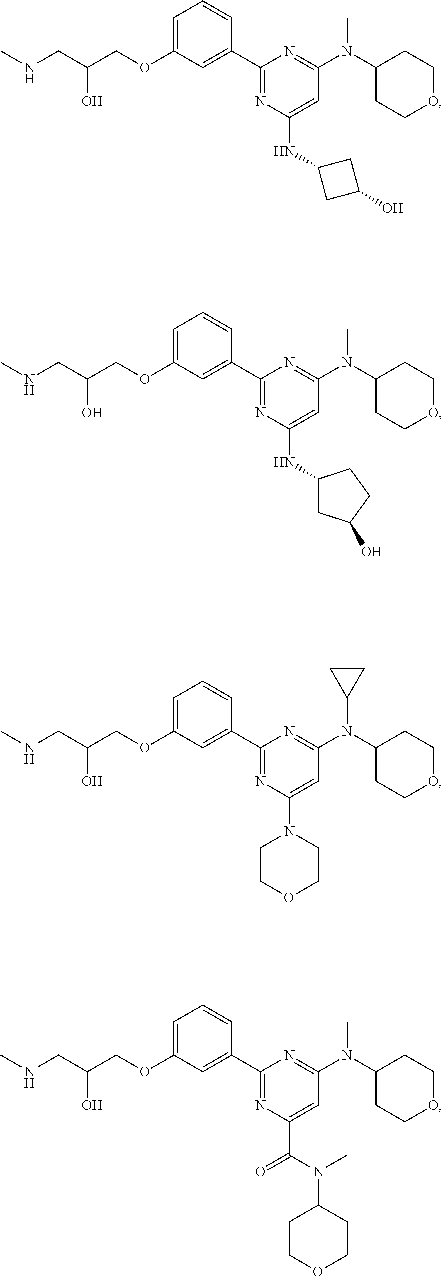 [SMILES: CNCC(O)COc1cccc(-c2nc(N[C@H]3C[C@@H](O)C3)cc(N(C)C3CCOCC3)n2)c1 ; CNCC(O)COc1cccc(-c2nc(N[C@@H]3CC[C@@H](O)C3)cc(N(C)C3CCOCC3)n2)c1 ; CNCC(O)COc1cccc(-c2nc(N3CCOCC3)cc(N(C3CCOCC3)C3CC3)n2)c1 ; CNCC(O)COc1cccc(-c2nc(C(=O)N(C)C3CCOCC3)cc(N(C)C3CCOCC3)n2)c1]

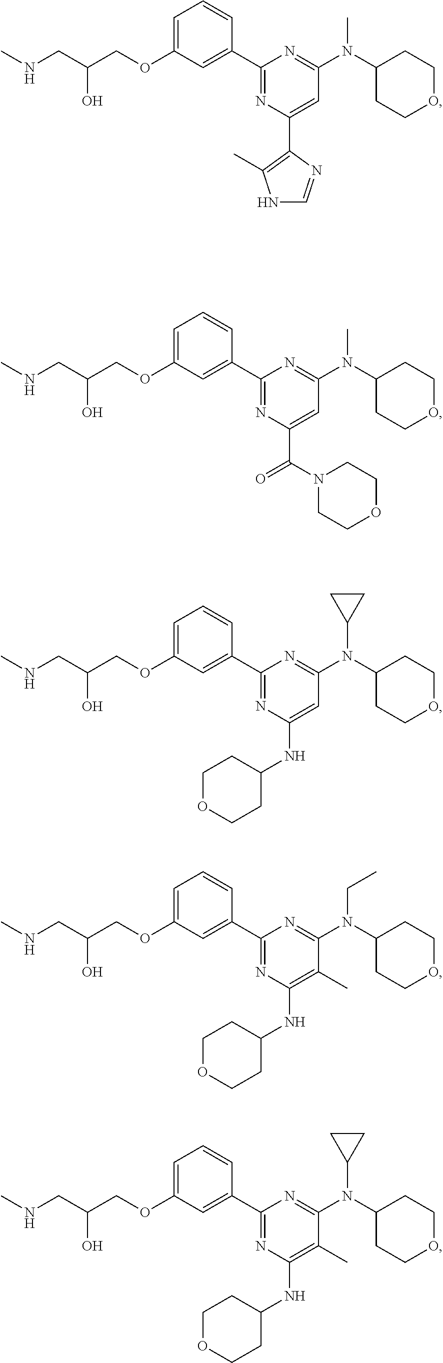 [SMILES: CNCC(O)COc1cccc(-c2nc(-c3nc[nH]c3C)cc(N(C)C3CCOCC3)n2)c1 ; CNCC(O)COc1cccc(-c2nc(C(=O)N3CCOCC3)cc(N(C)C3CCOCC3)n2)c1 ; CNCC(O)COc1cccc(-c2nc(NC3CCOCC3)cc(N(C3CCOCC3)C3CC3)n2)c1 ; CCN(c1nc(-c2cccc(OCC(O)CNC)c2)nc(NC2CCOCC2)c1C)C1CCOCC1 ; CNCC(O)COc1cccc(-c2nc(NC3CCOCC3)c(C)c(N(C3CCOCC3)C3CC3)n2)c1]

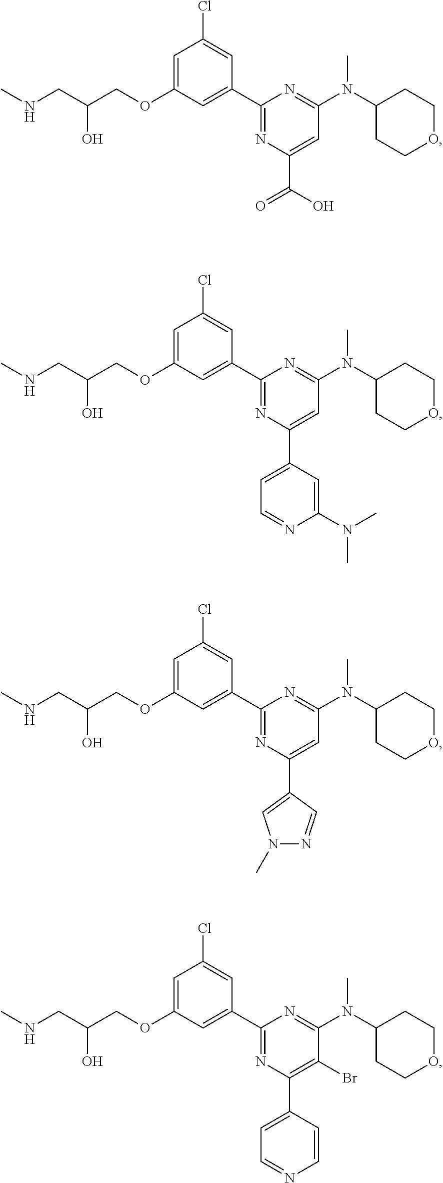 [SMILES: CNCC(O)COc1cc(Cl)cc(-c2nc(C(=O)O)cc(N(C)C3CCOCC3)n2)c1 ; CNCC(O)COc1cc(Cl)cc(-c2nc(-c3ccnc(N(C)C)c3)cc(N(C)C3CCOCC3)n2)c1 ; CNCC(O)COc1cc(Cl)cc(-c2nc(-c3cnn(C)c3)cc(N(C)C3CCOCC3)n2)c1 ; CNCC(O)COc1cc(Cl)cc(-c2nc(-c3ccncc3)c(Br)c(N(C)C3CCOCC3)n2)c1]

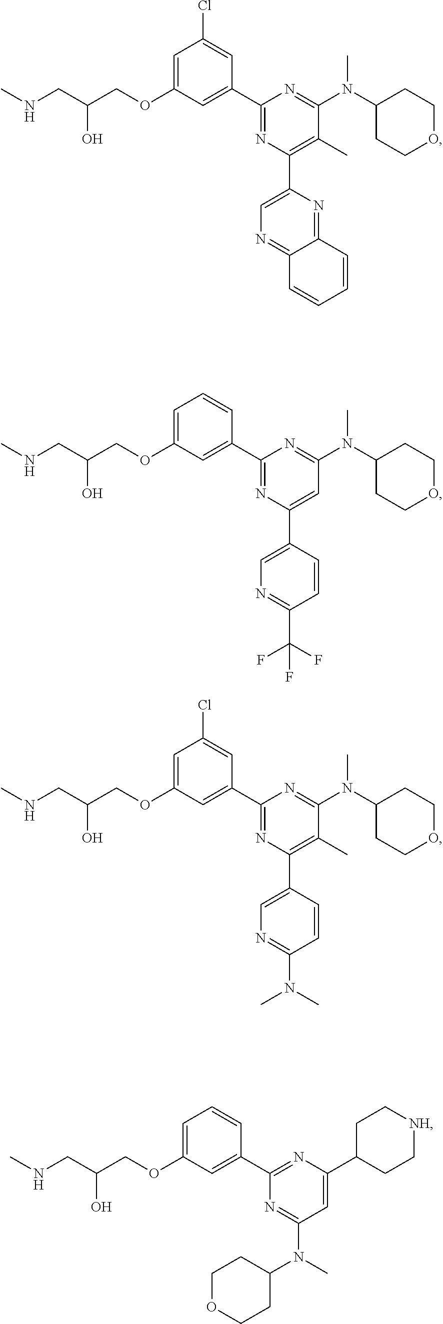 [SMILES: CNCC(O)COc1cc(Cl)cc(-c2nc(-c3cnc4ccccc4n3)c(C)c(N(C)C3CCOCC3)n2)c1 ; CNCC(O)COc1cccc(-c2nc(-c3ccc(C(F)(F)F)nc3)cc(N(C)C3CCOCC3)n2)c1 ; CNCC(O)COc1cc(Cl)cc(-c2nc(-c3ccc(N(C)C)nc3)c(C)c(N(C)C3CCOCC3)n2)c1 ; CNCC(O)COc1cccc(-c2nc(C3CCNCC3)cc(N(C)C3CCOCC3)n2)c1]

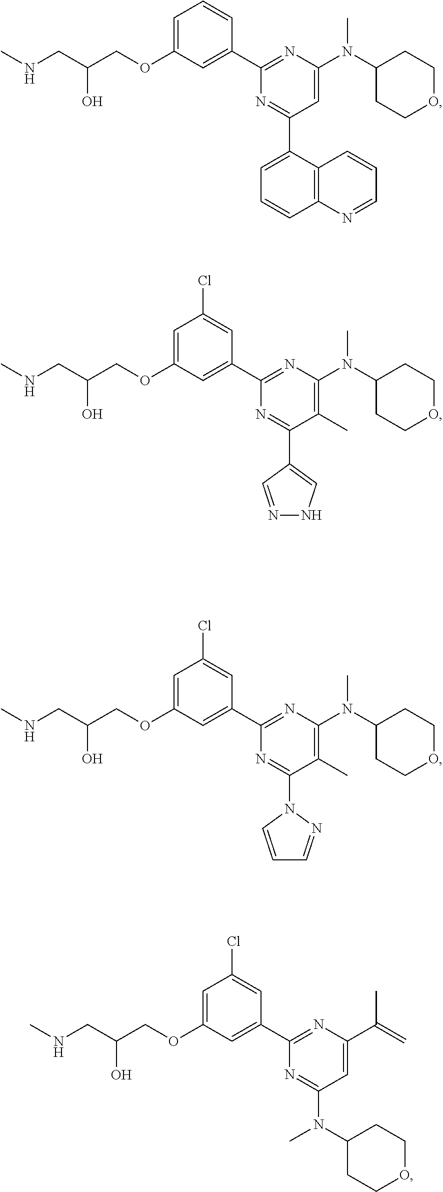 [SMILES: CNCC(O)COc1cccc(-c2nc(-c3cccc4ncccc34)cc(N(C)C3CCOCC3)n2)c1 ; CNCC(O)COc1cc(Cl)cc(-c2nc(-c3cn[nH]c3)c(C)c(N(C)C3CCOCC3)n2)c1 ; CNCC(O)COc1cc(Cl)cc(-c2nc(N(C)C3CCOCC3)c(C)c(-n3cccn3)n2)c1 ; C=C(C)c1cc(N(C)C2CCOCC2)nc(-c2cc(Cl)cc(OCC(O)CNC)c2)n1]

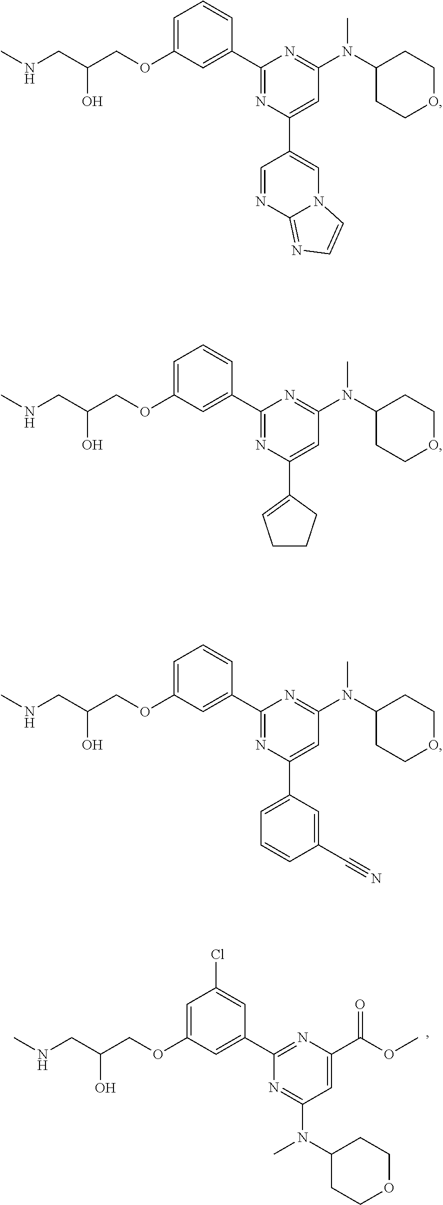

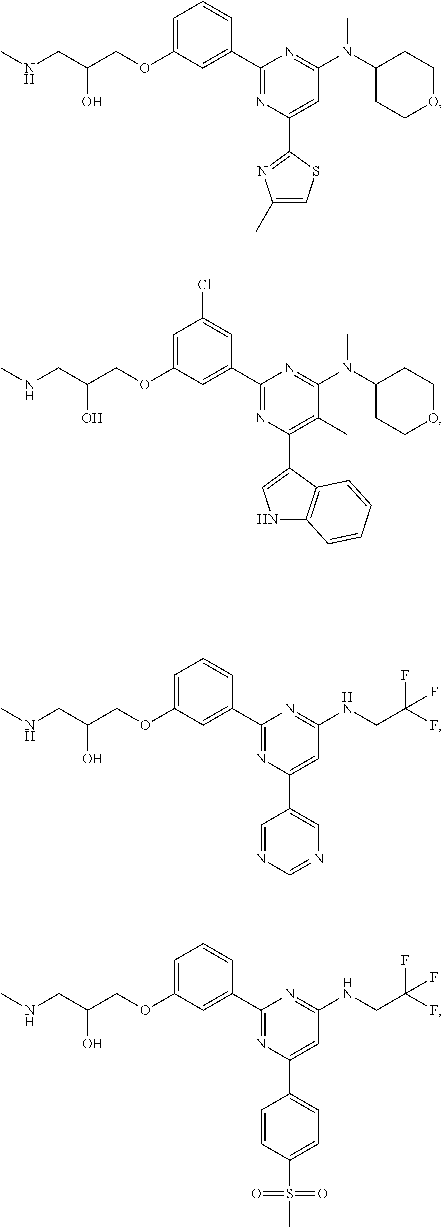 [SMILES: CNCC(O)COc1cccc(-c2nc(-c3nc(C)cs3)cc(N(C)C3CCOCC3)n2)c1 ; CNCC(O)COc1cc(Cl)cc(-c2nc(-c3c[nH]c4ccccc34)c(C)c(N(C)C3CCOCC3)n2)c1 ; CNCC(O)COc1cccc(-c2nc(NCC(F)(F)F)cc(-c3cncnc3)n2)c1 ; CNCC(O)COc1cccc(-c2nc(NCC(F)(F)F)cc(-c3ccc(S(C)(=O)=O)cc3)n2)c1]

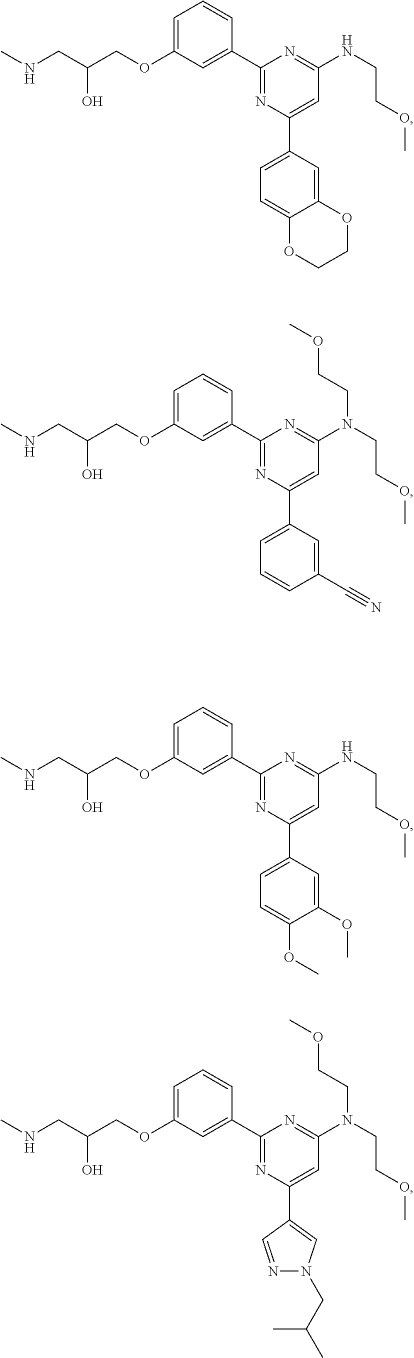

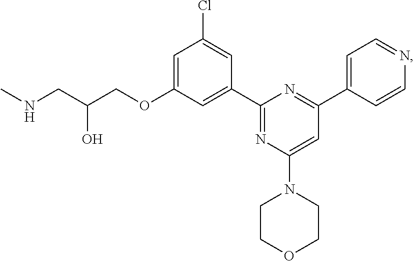 [SMILES: CNCC(O)COc1cc(Cl)cc(-c2nc(-c3ccncc3)cc(N3CCOCC3)n2)c1]

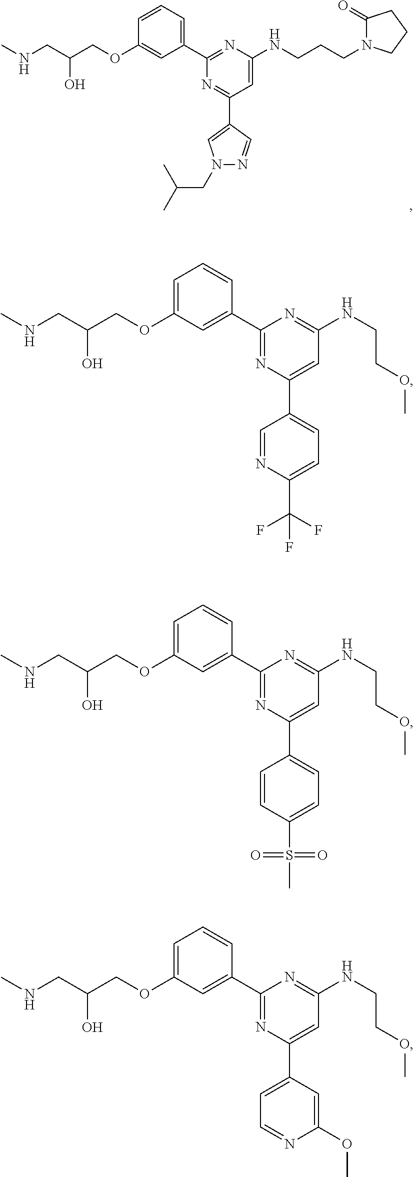

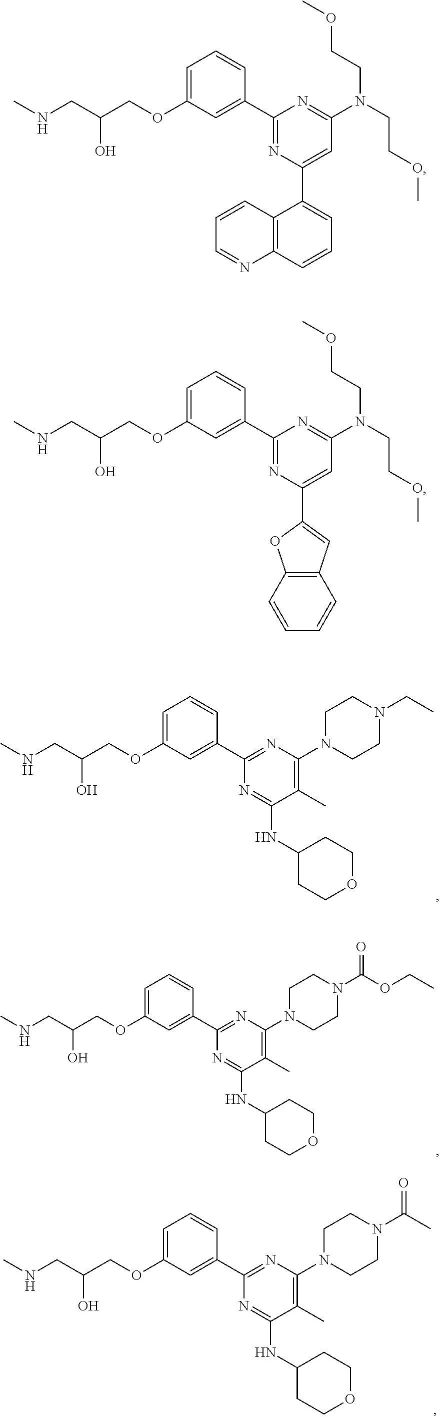

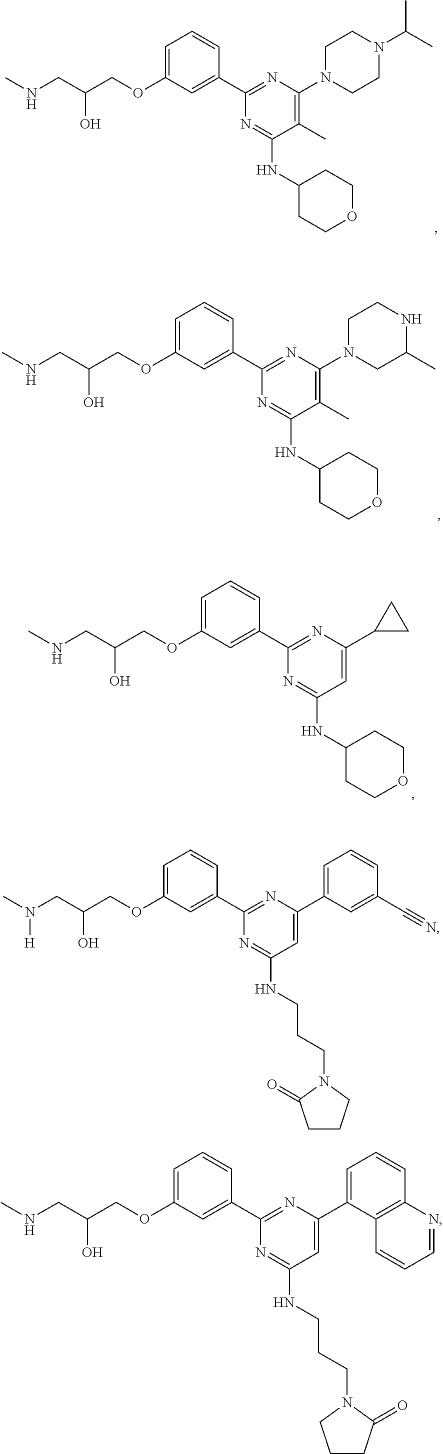

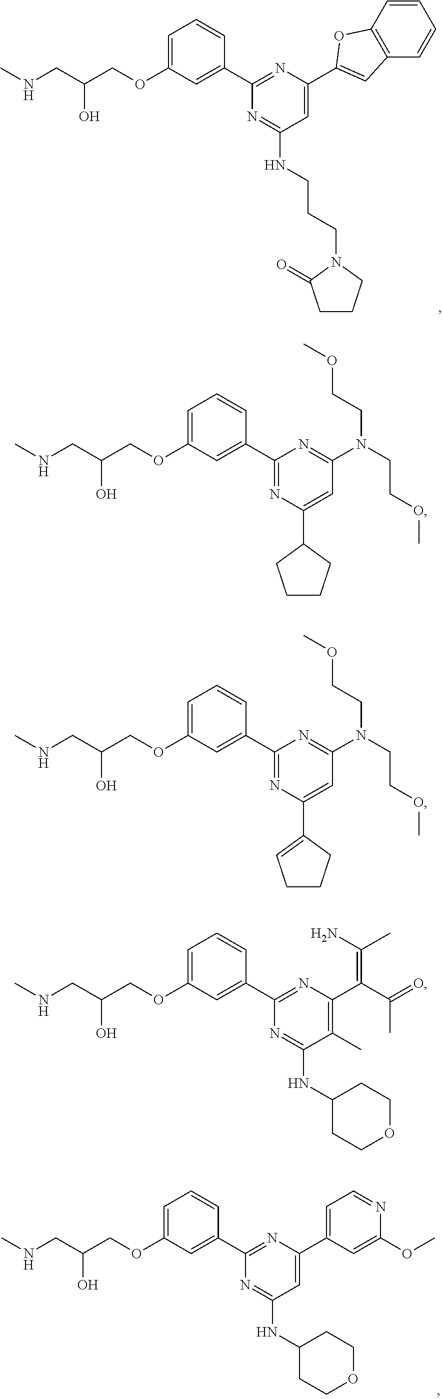

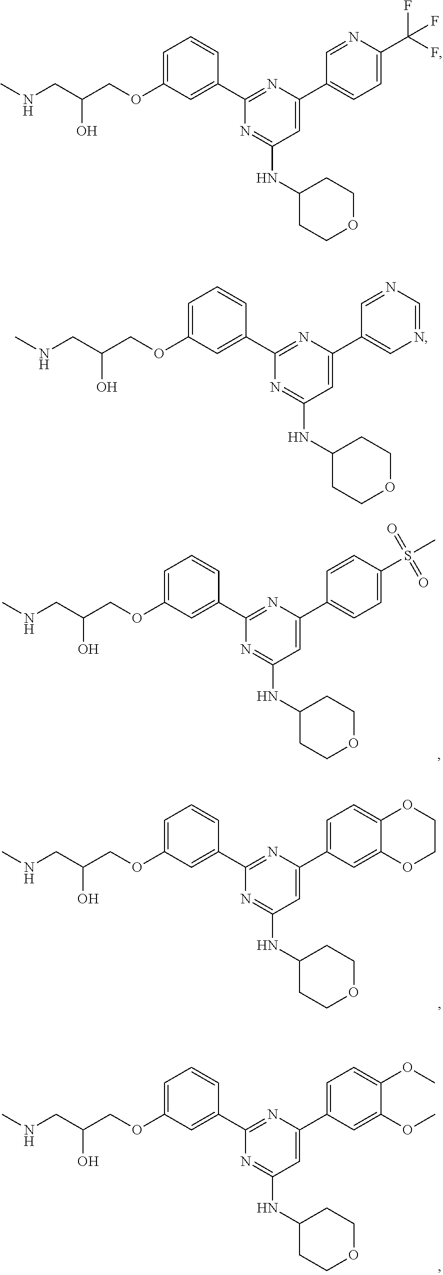

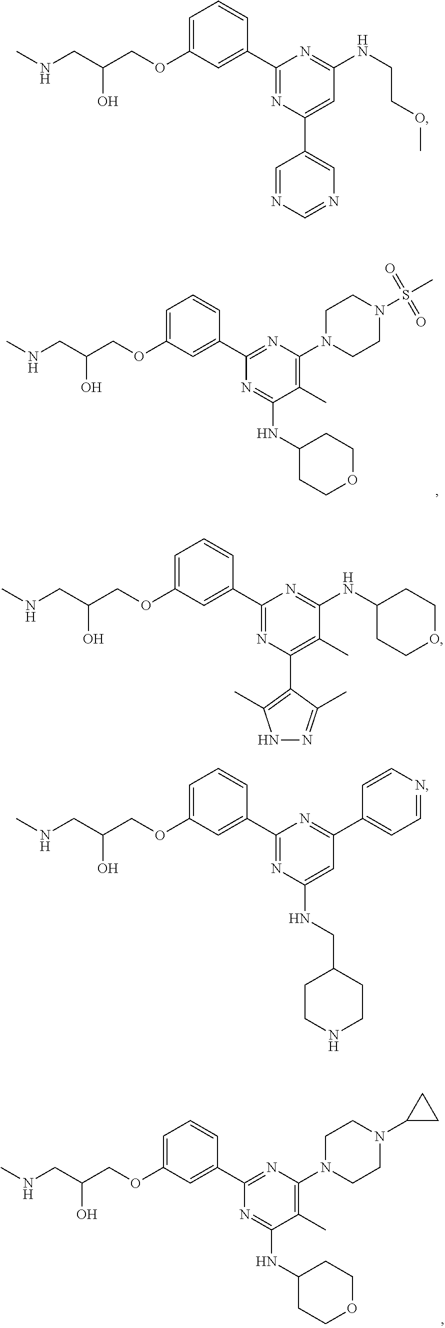

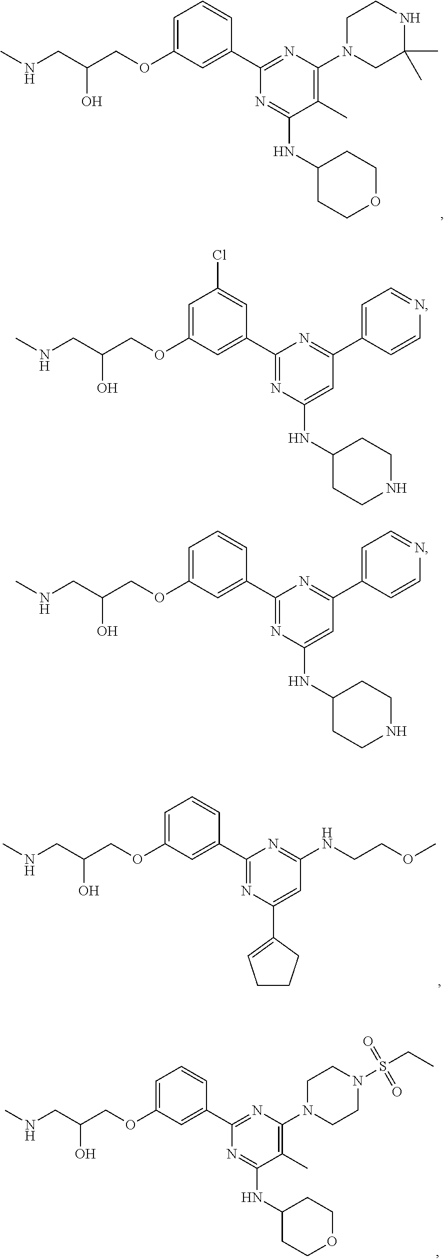

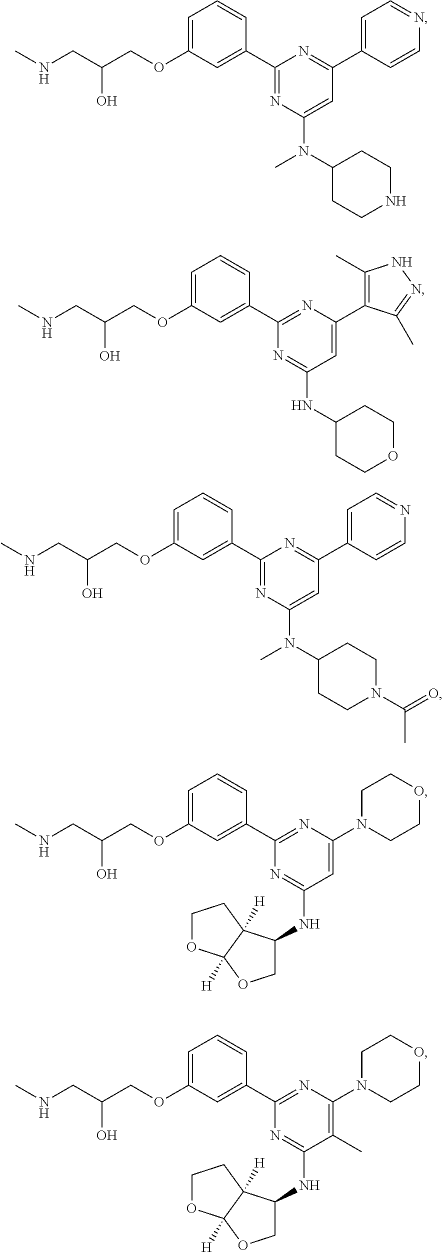

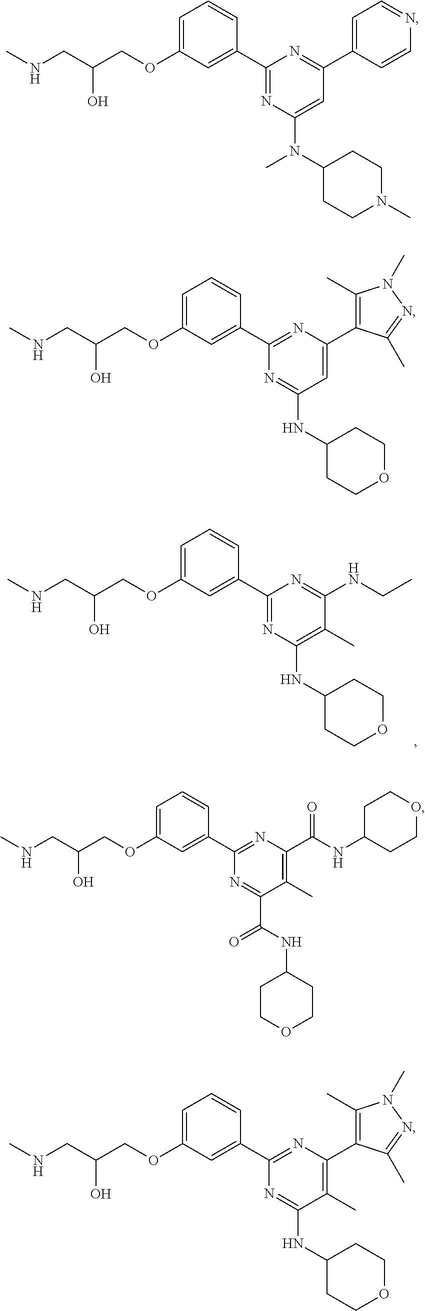

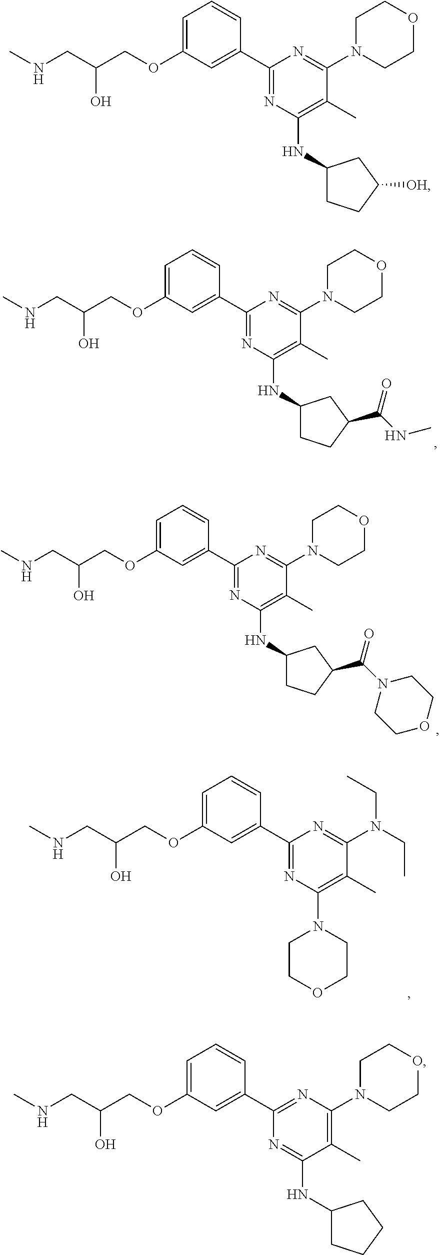

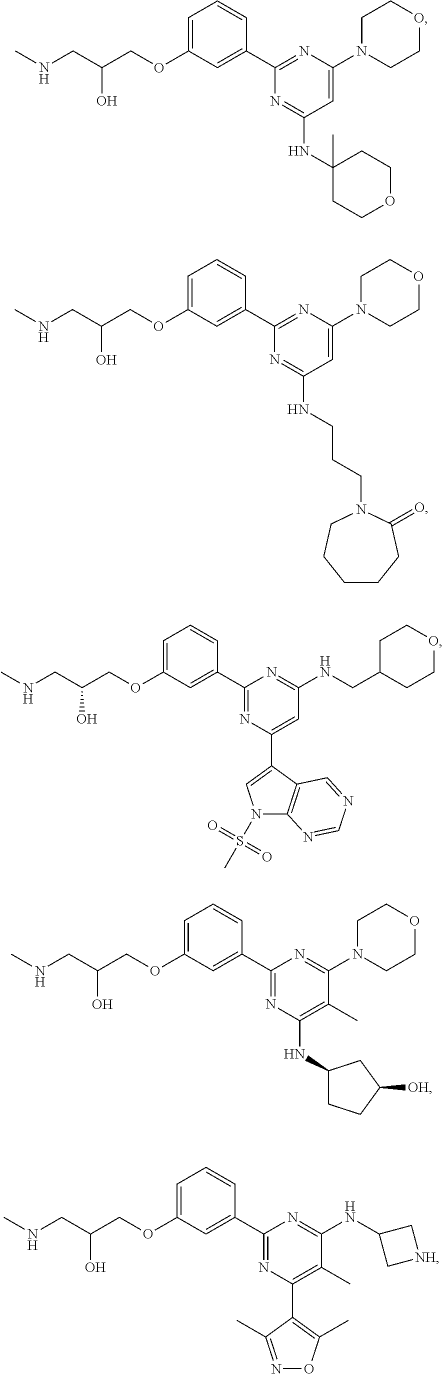

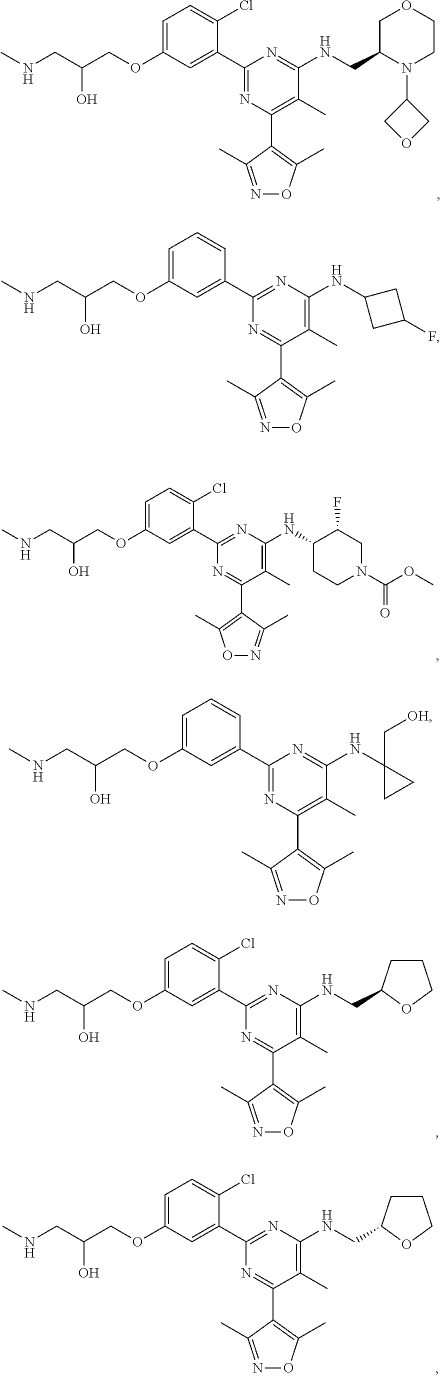

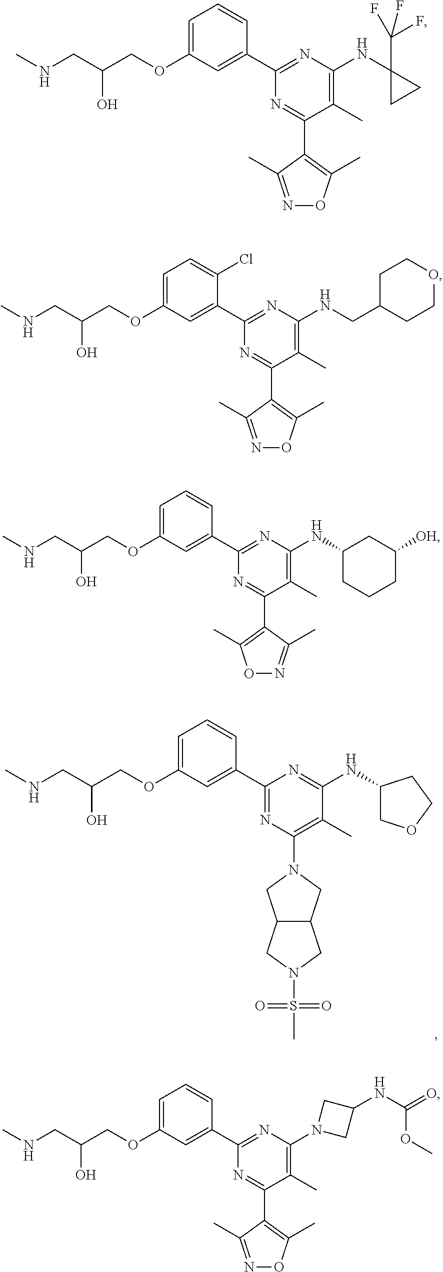

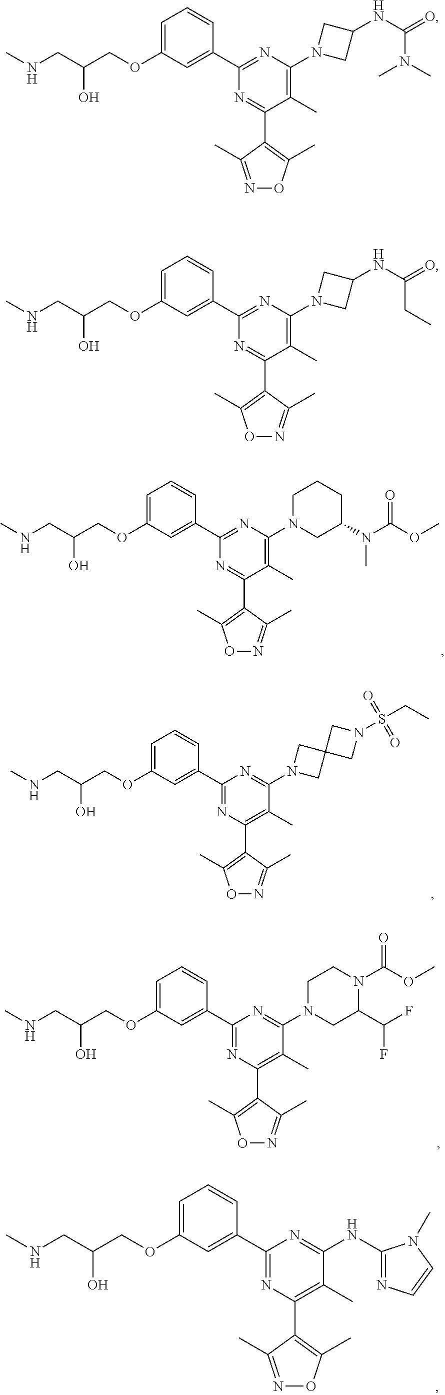

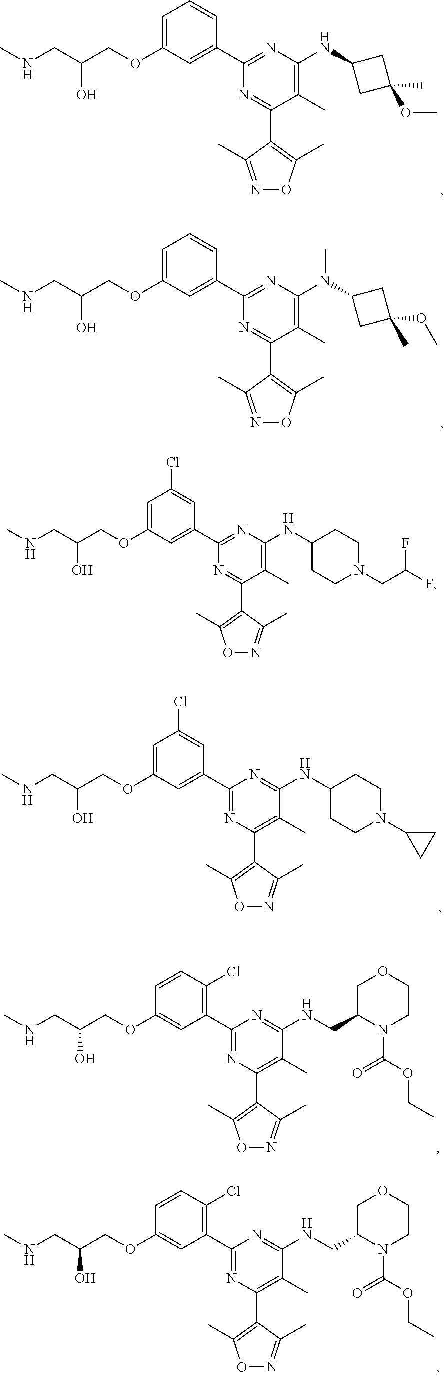

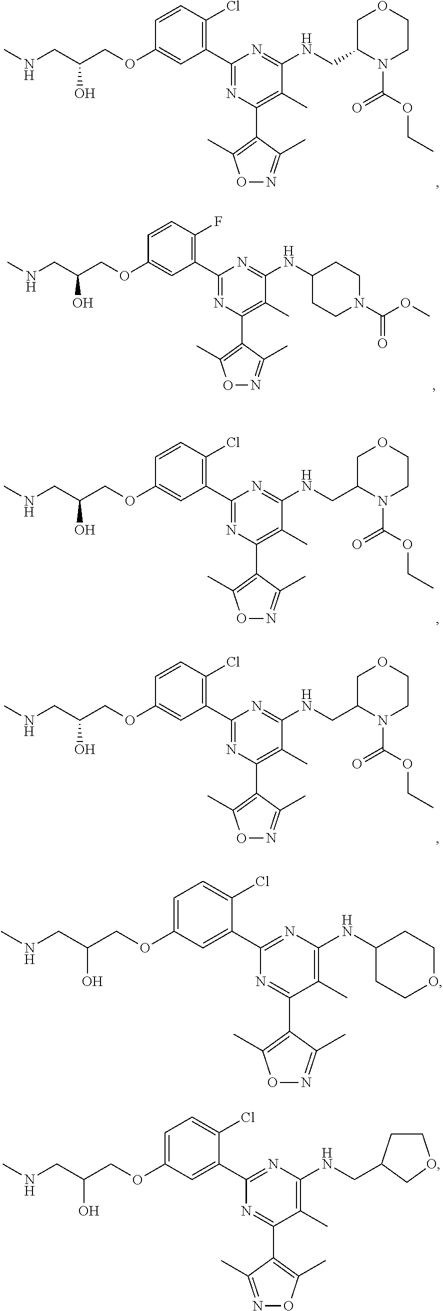

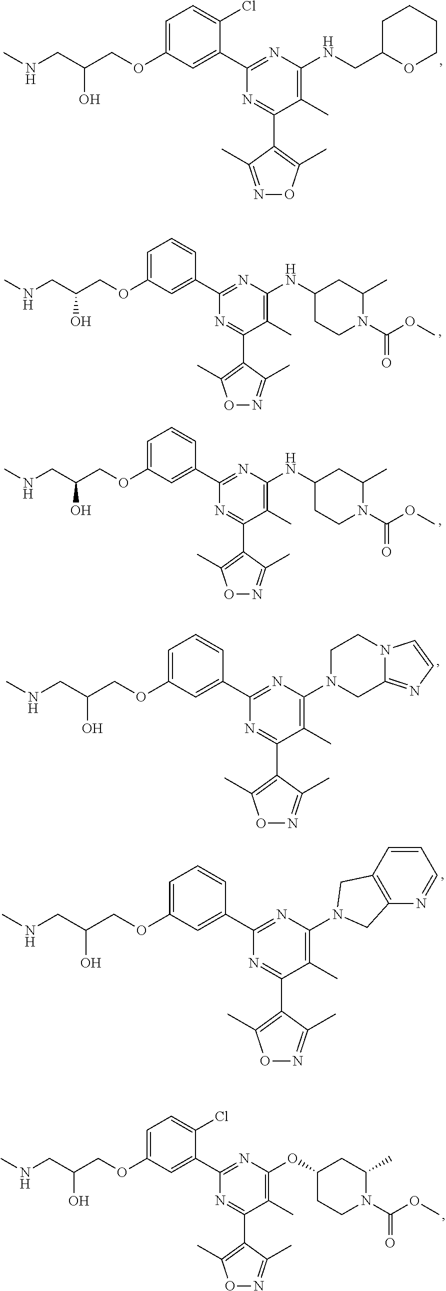

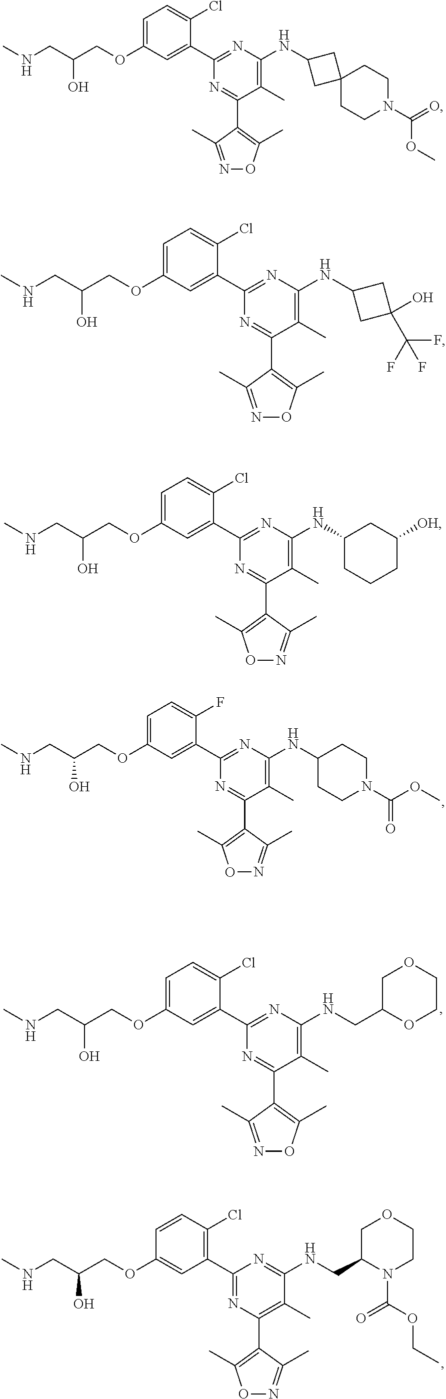

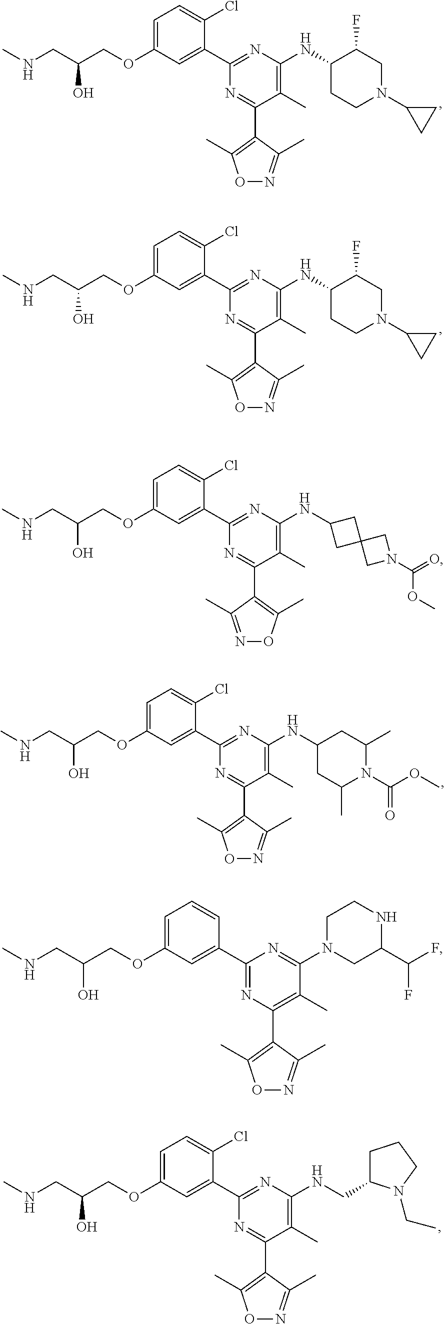

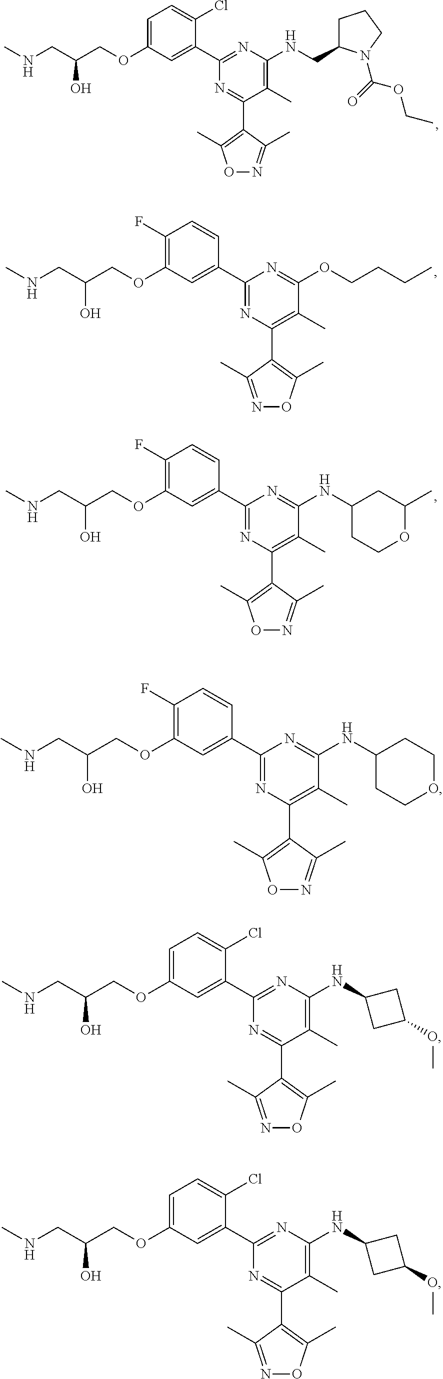

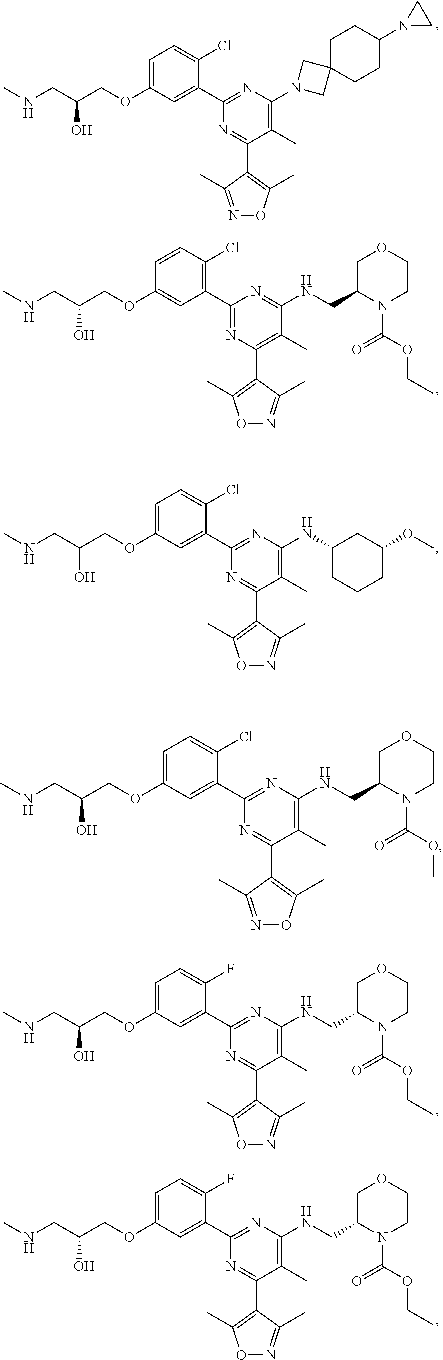

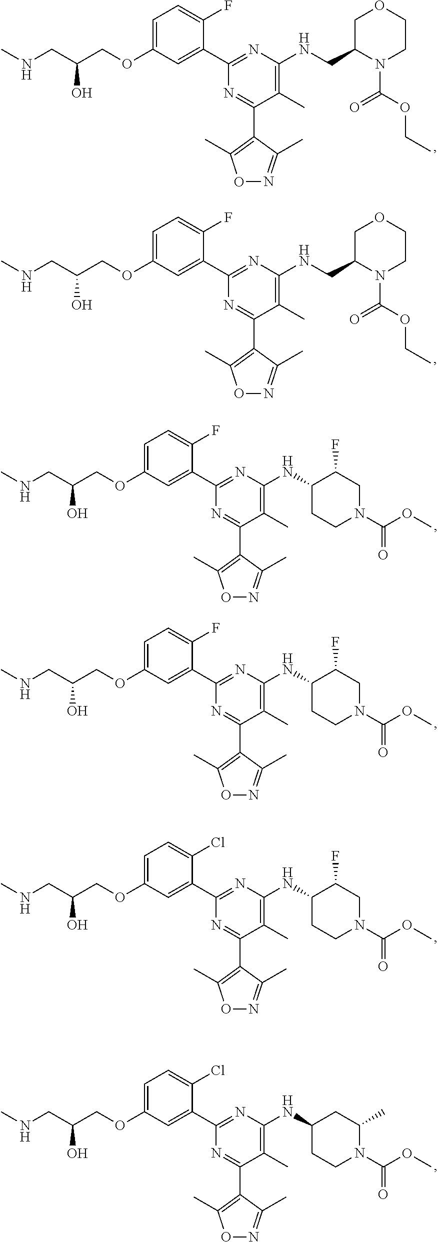

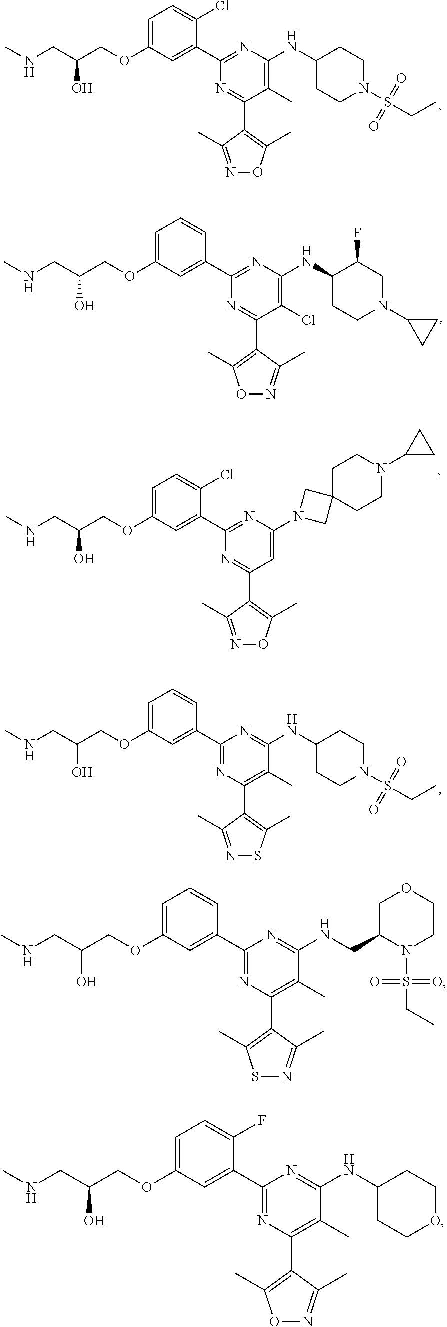

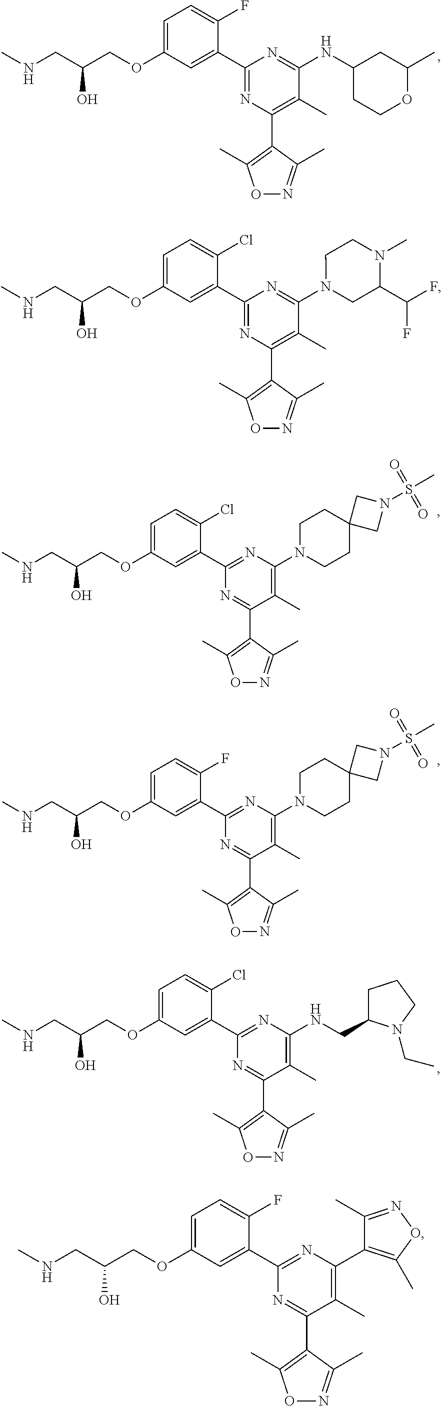

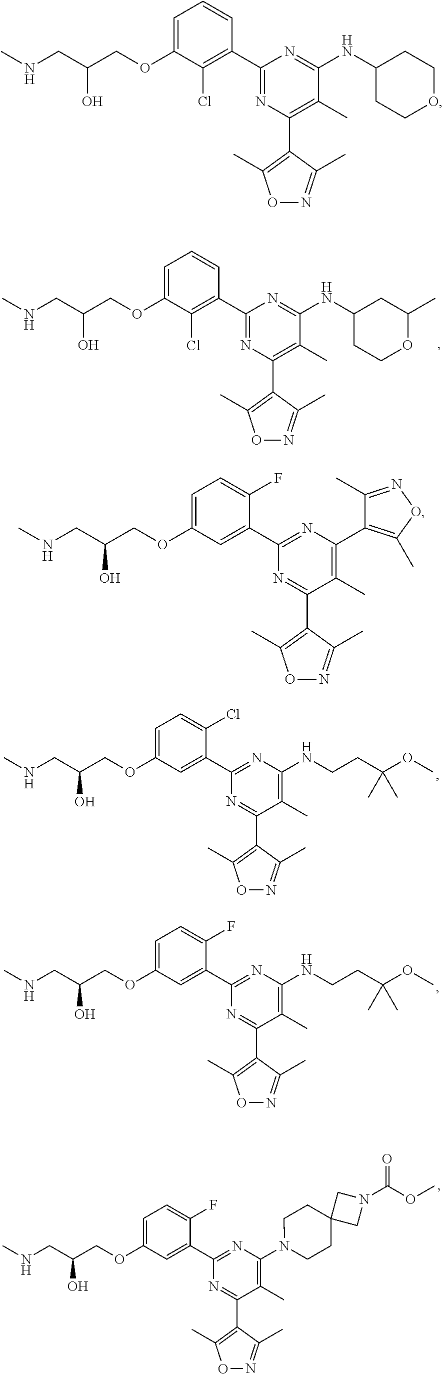

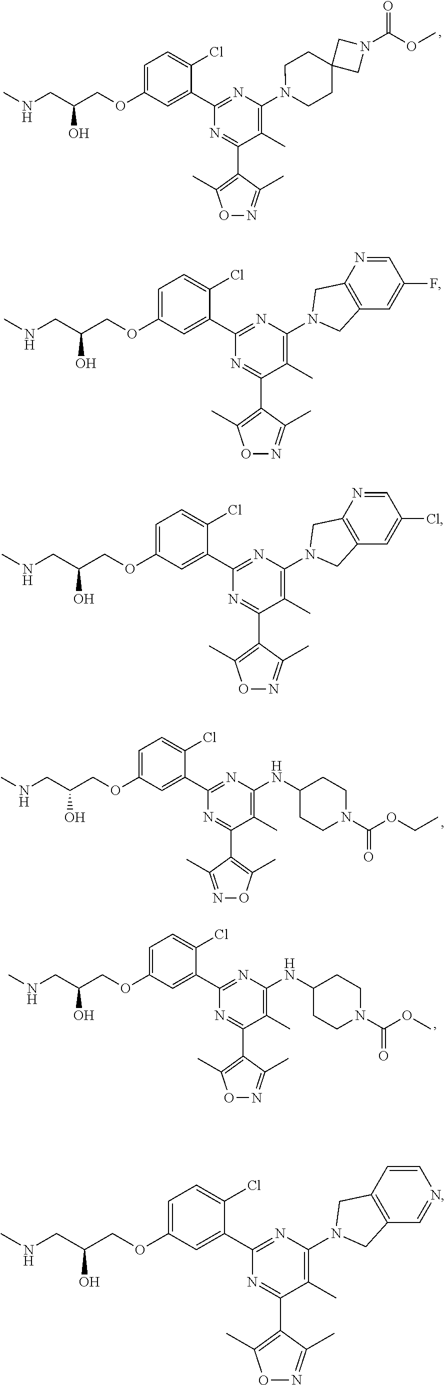

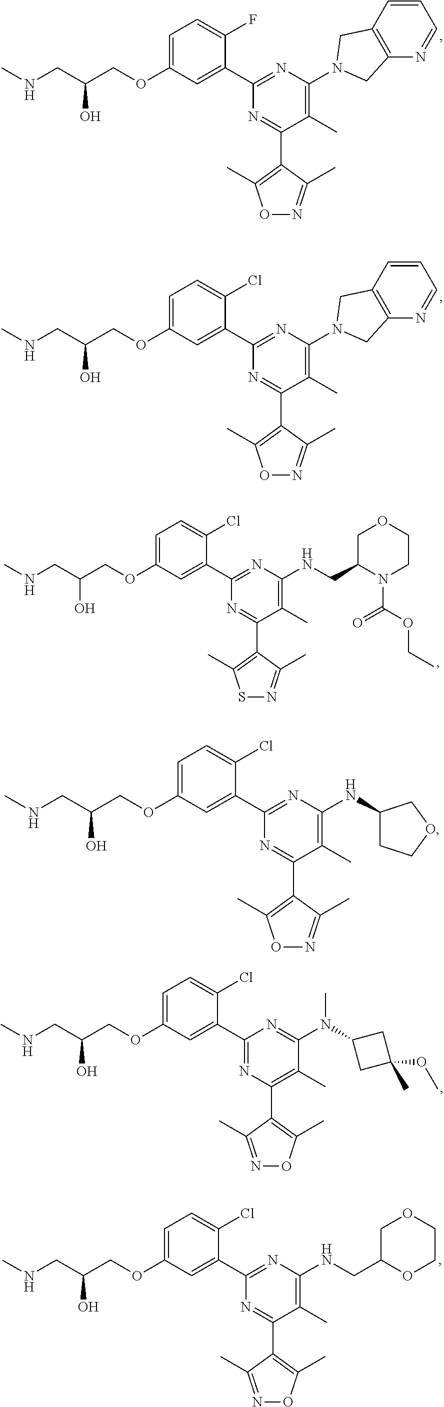

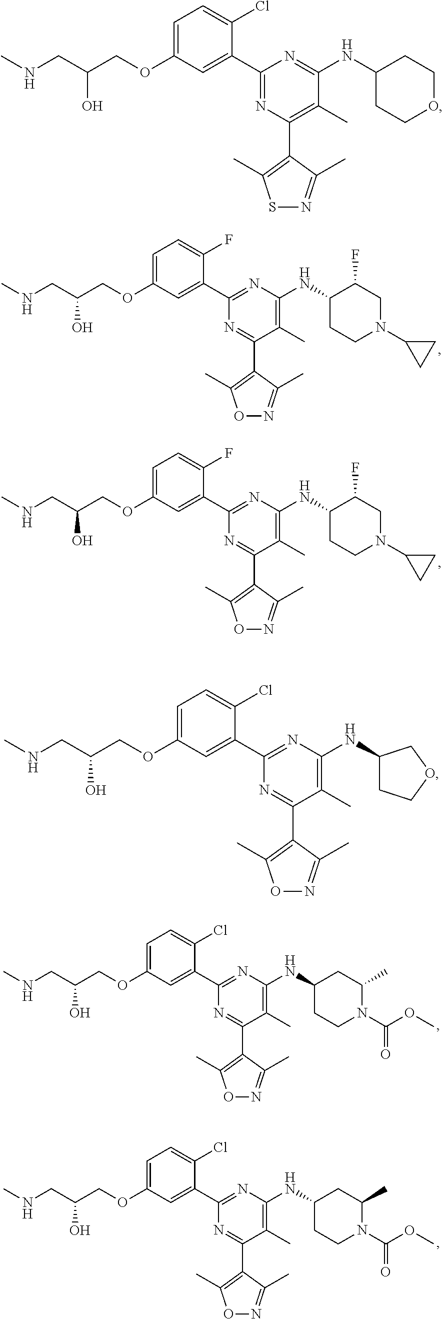

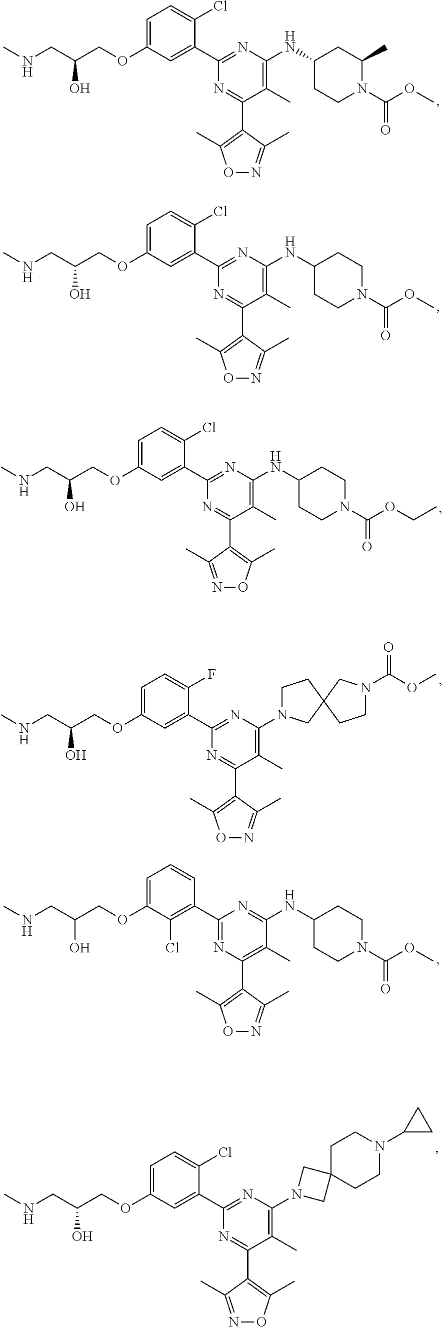

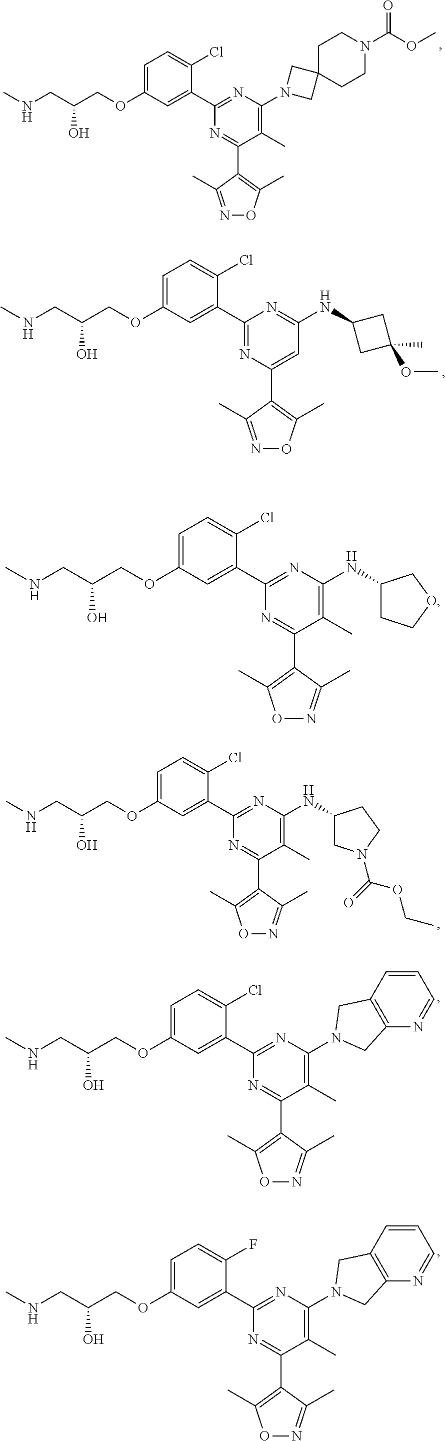

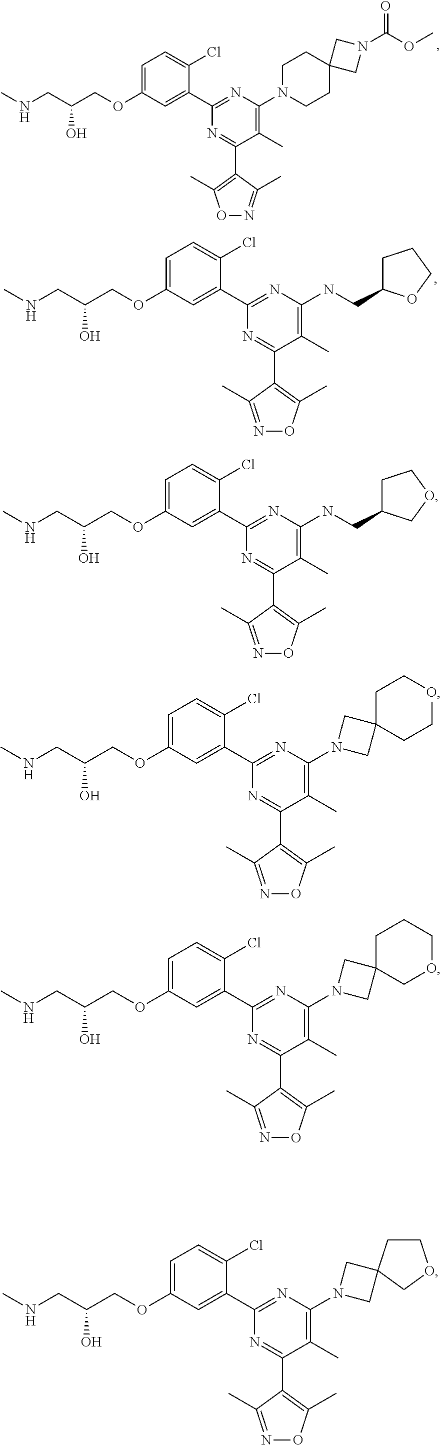

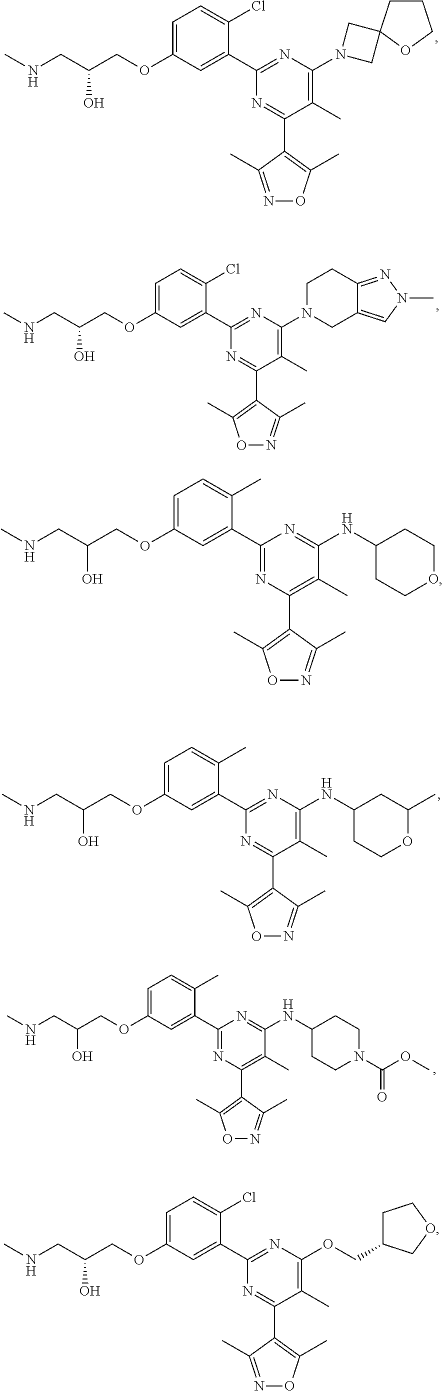

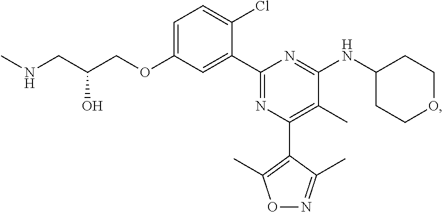 [SMILES: CNC[C@@H](O)COc1ccc(Cl)c(-c2nc(NC3CCOCC3)c(C)c(-c3c(C)noc3C)n2)c1]

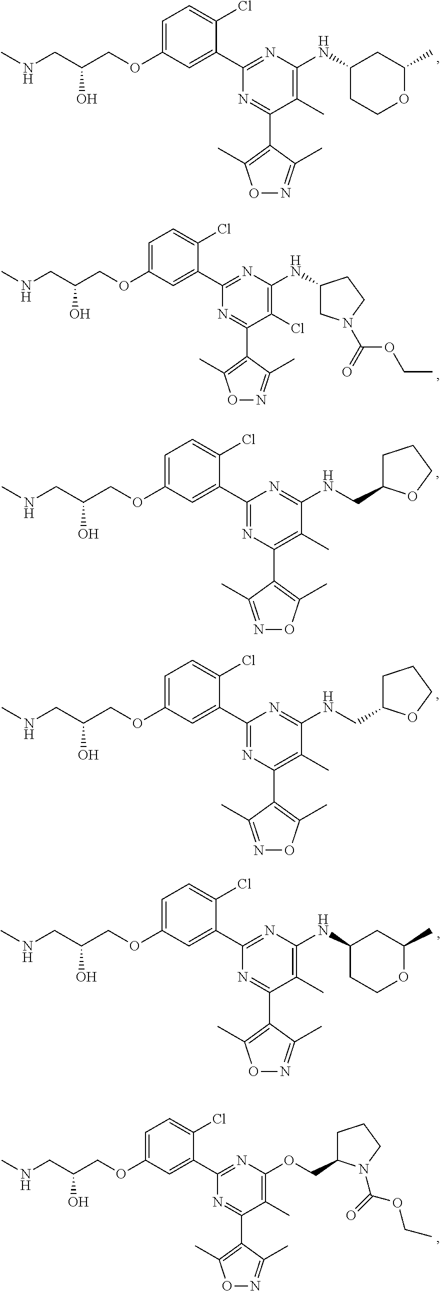

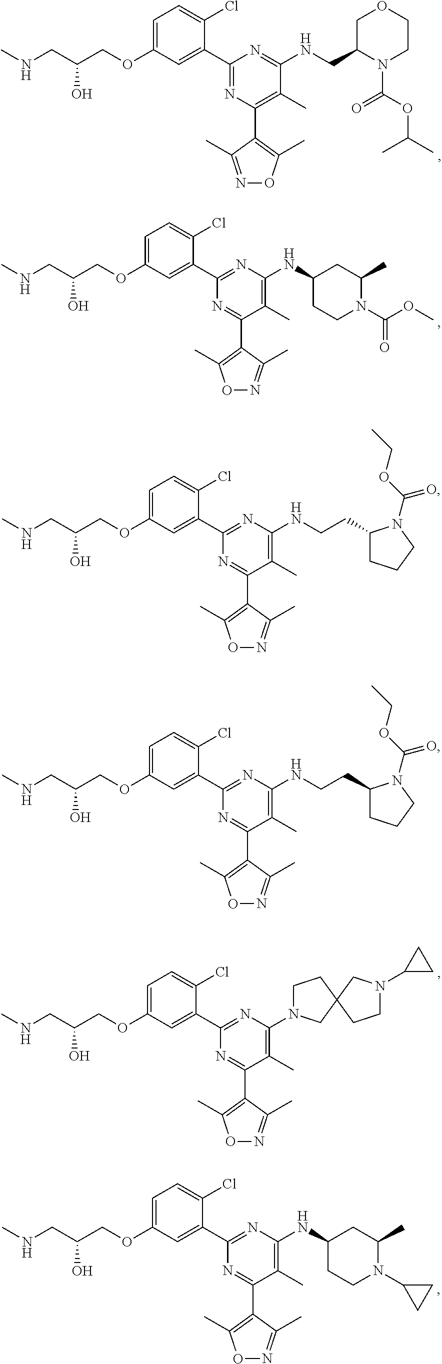

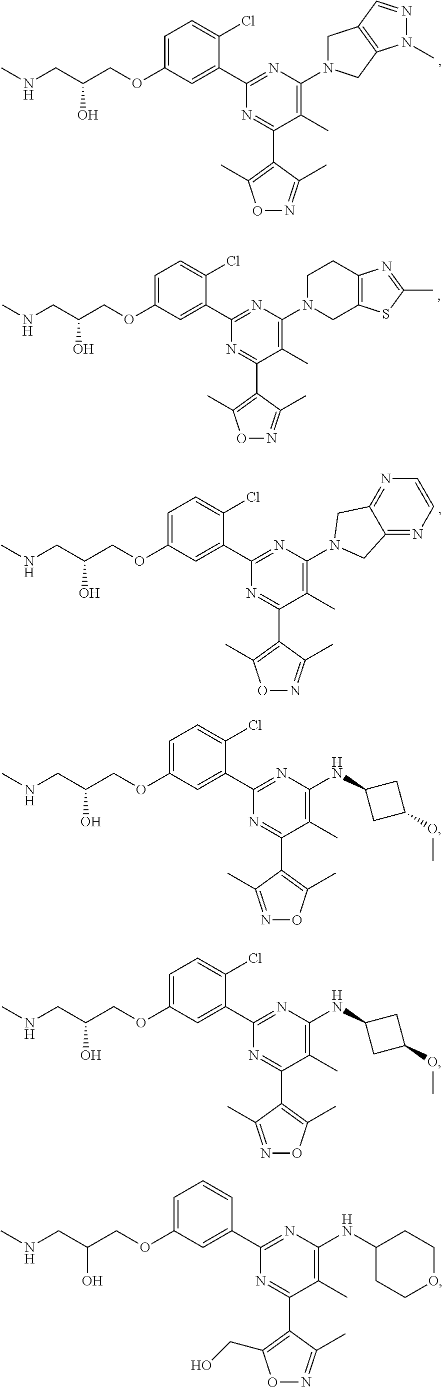

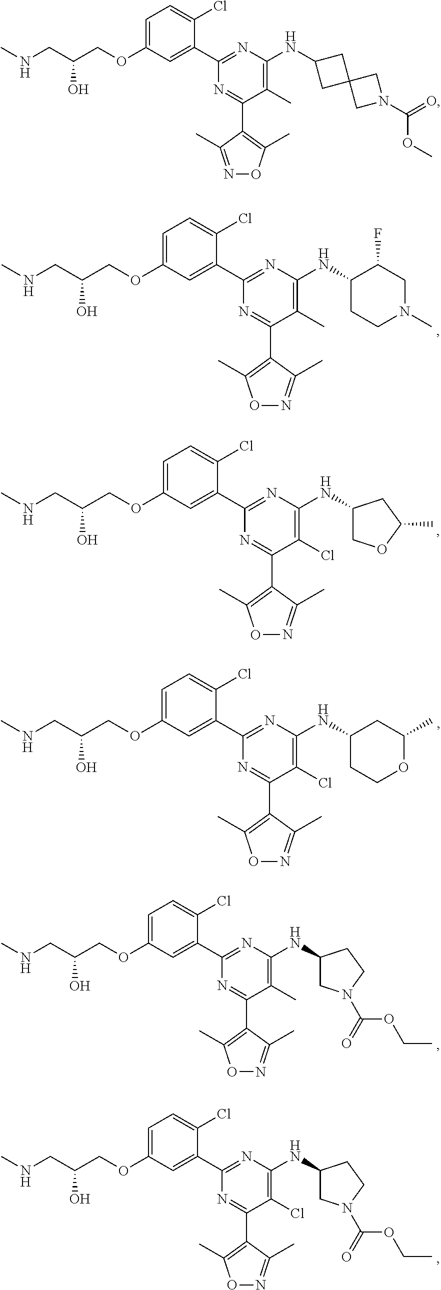

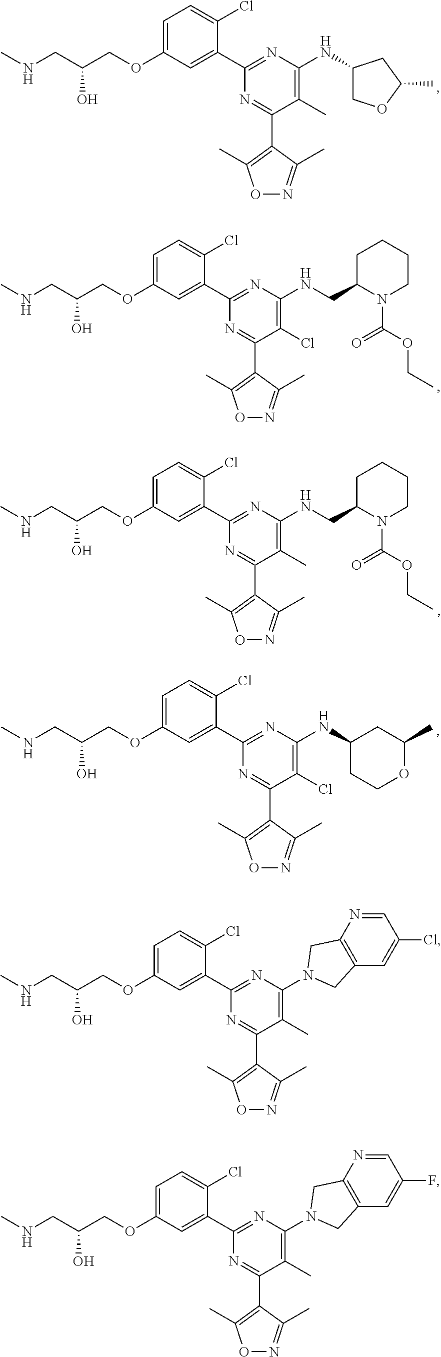

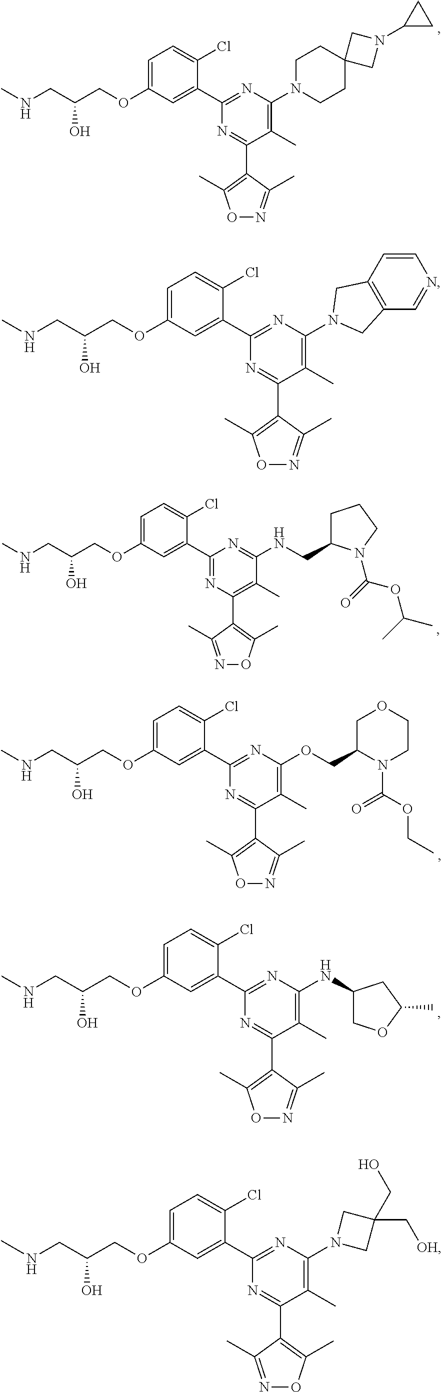

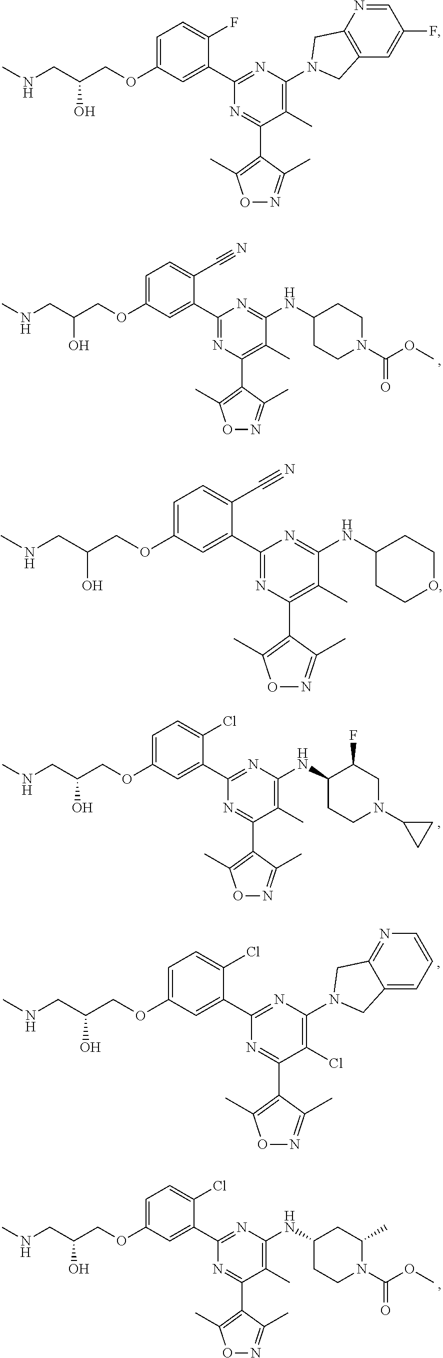

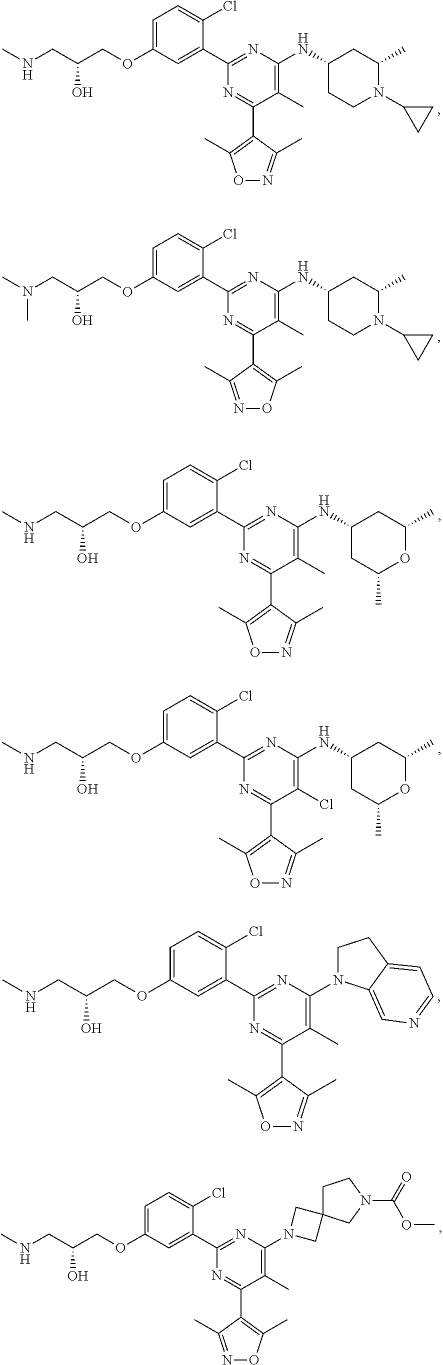

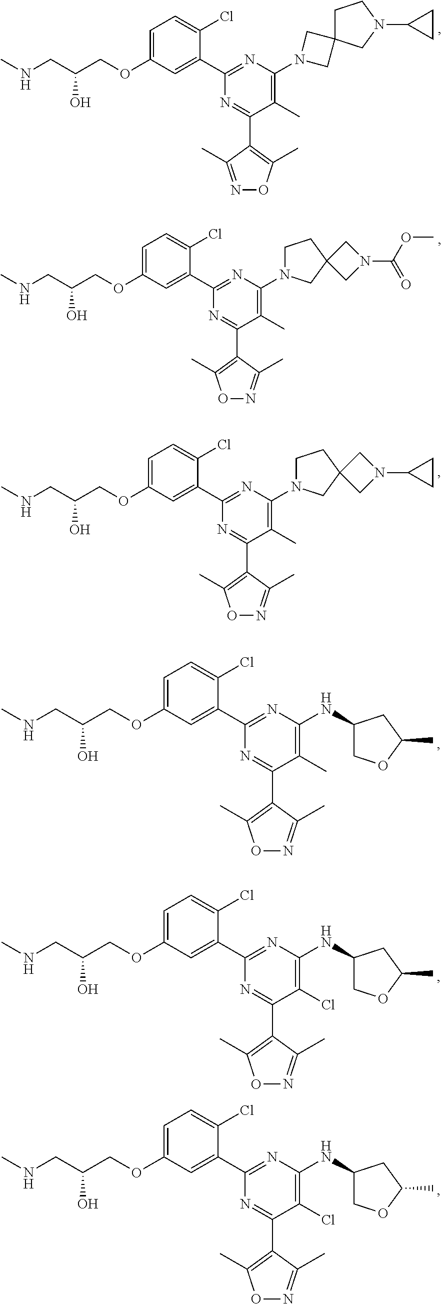

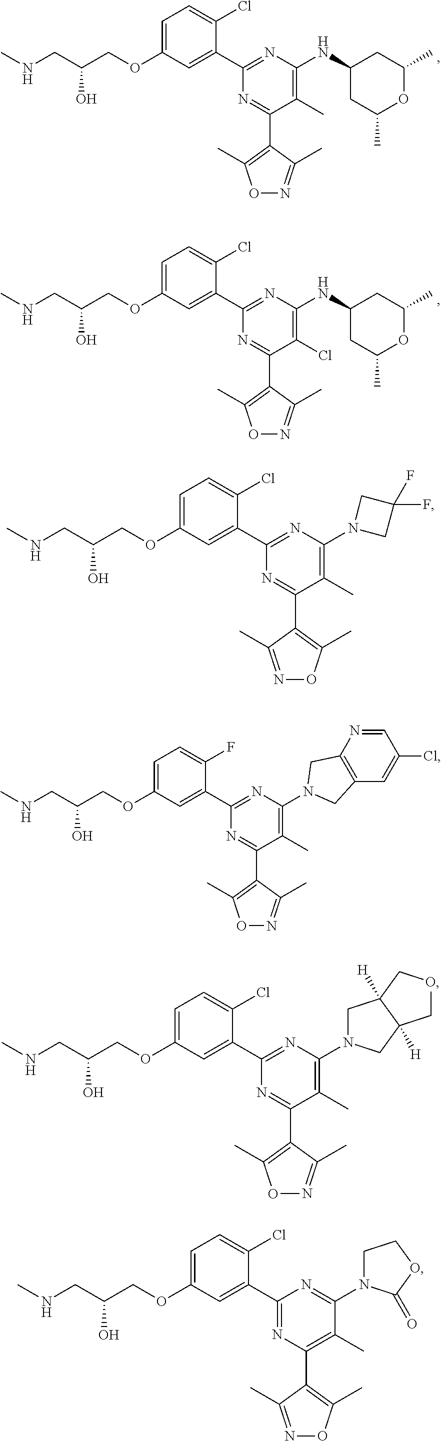

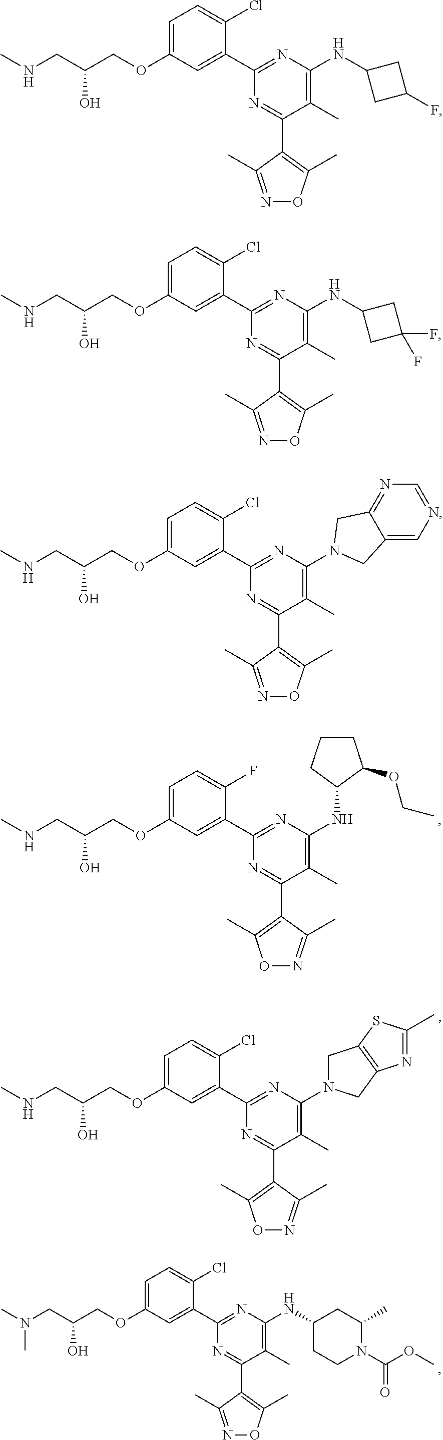

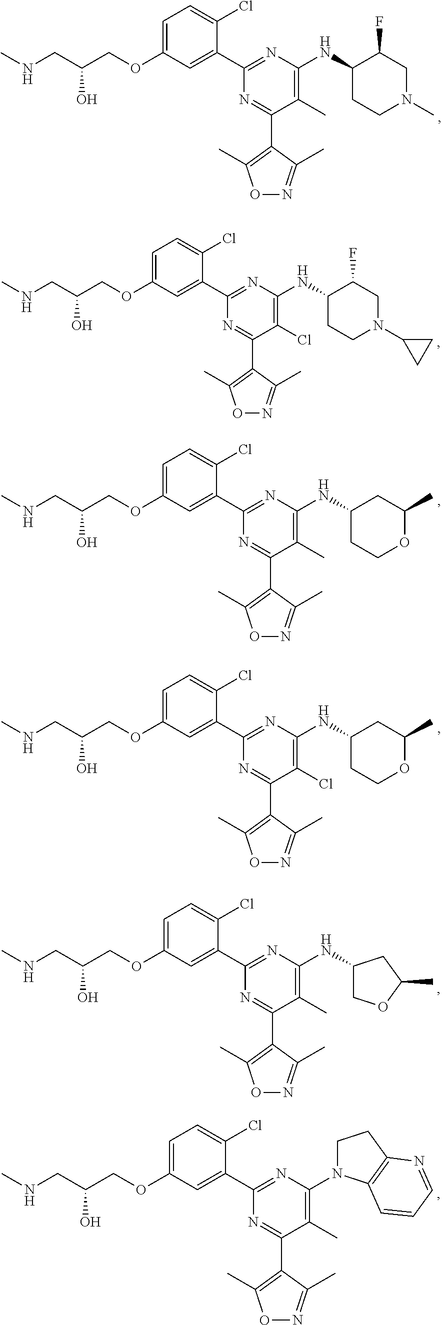

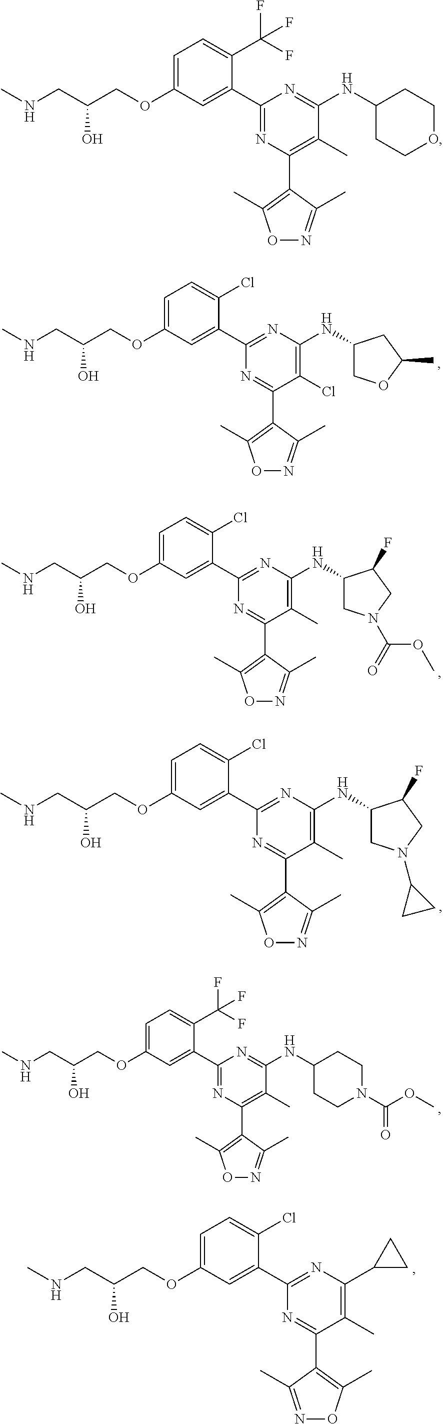

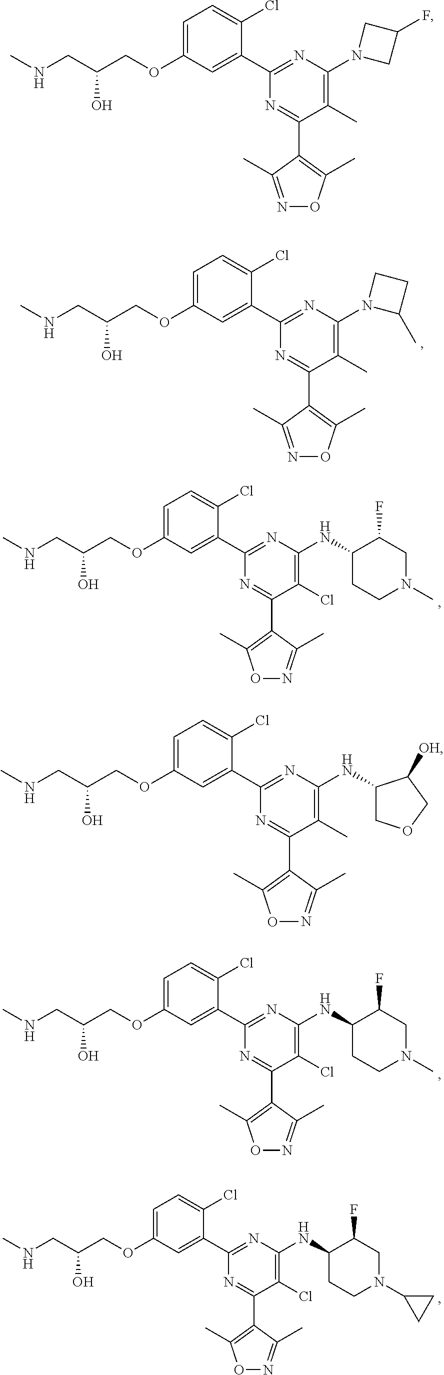

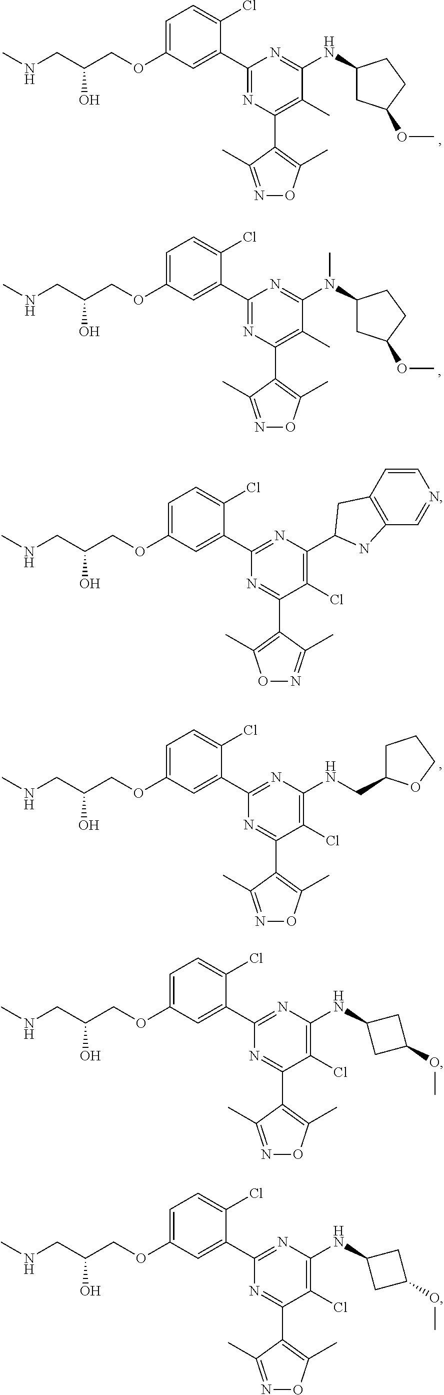

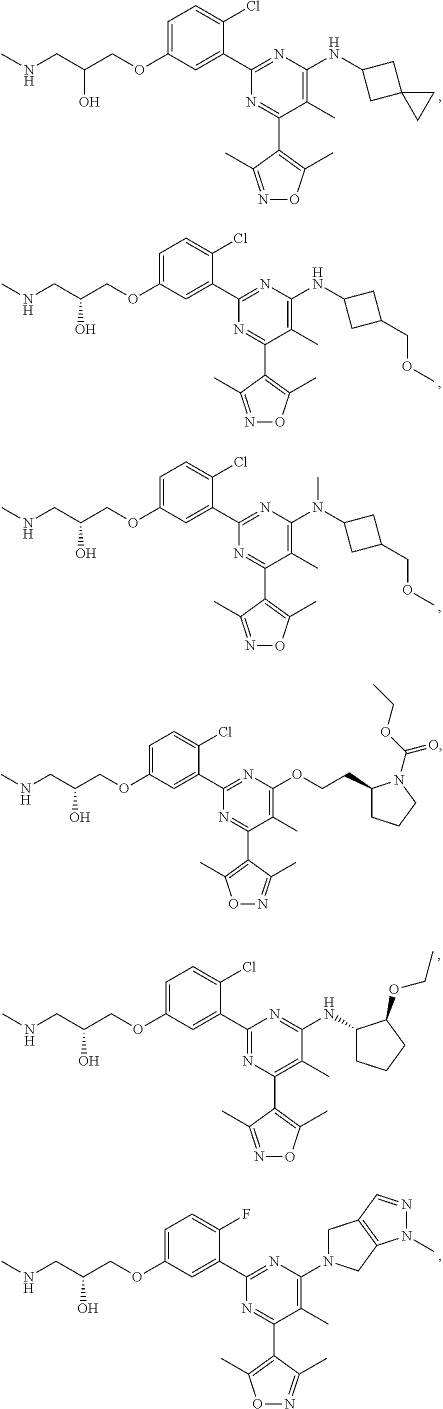

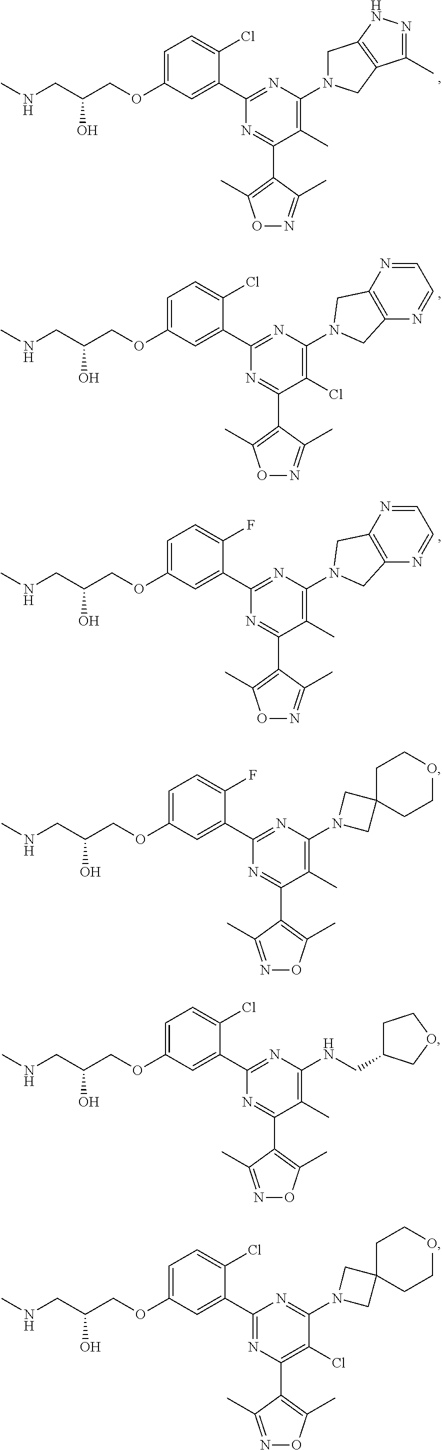

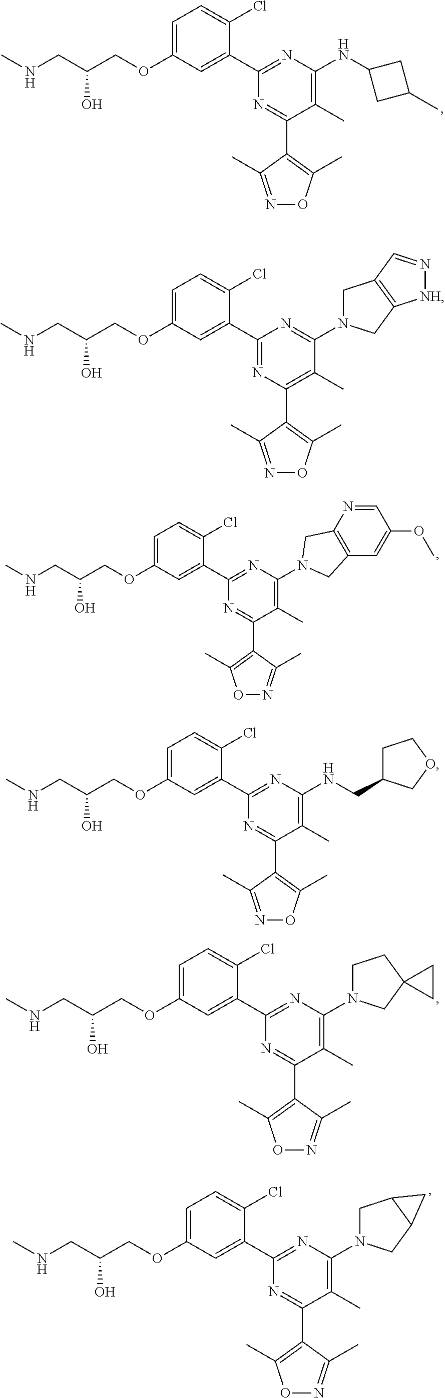

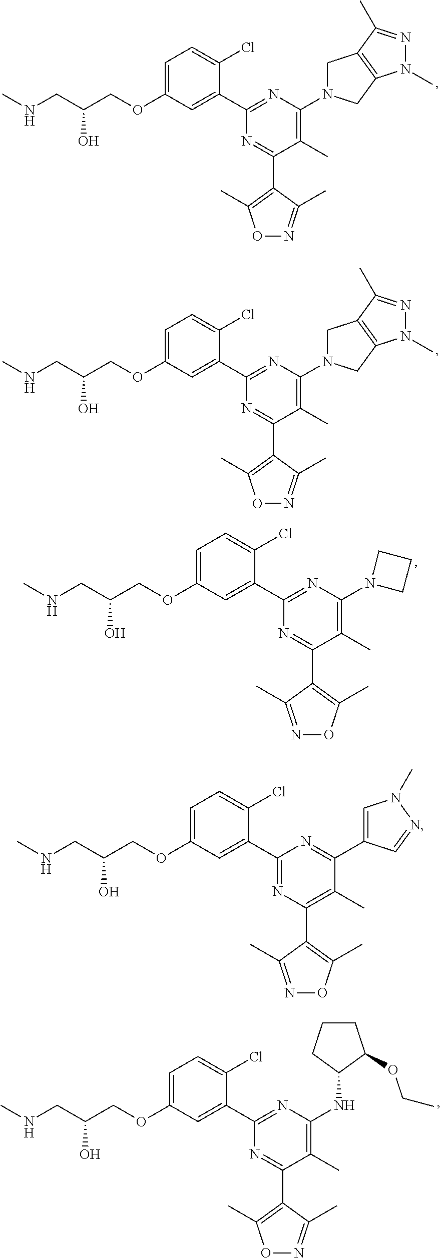

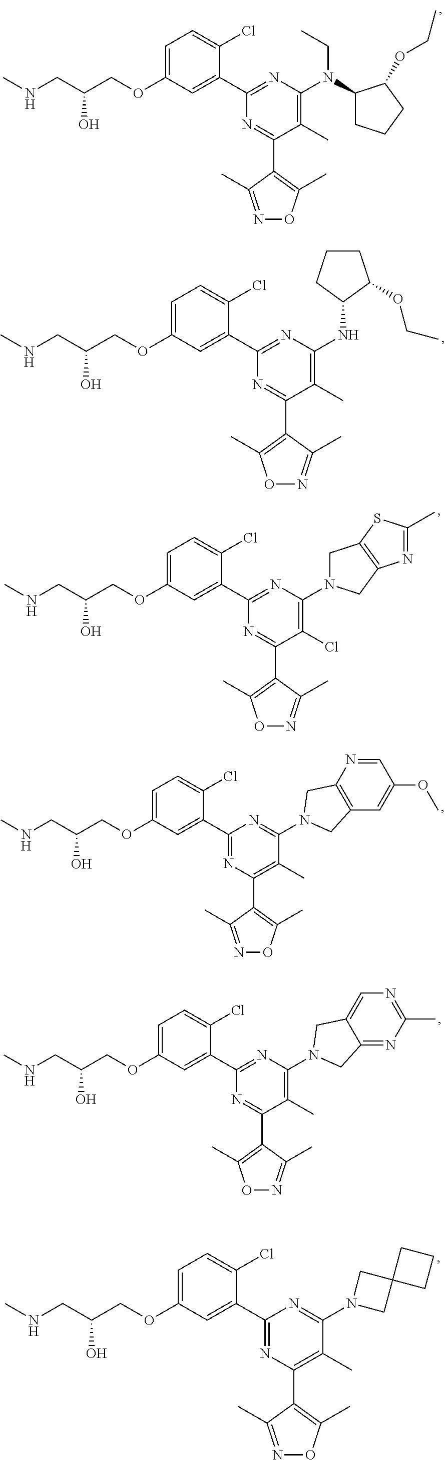

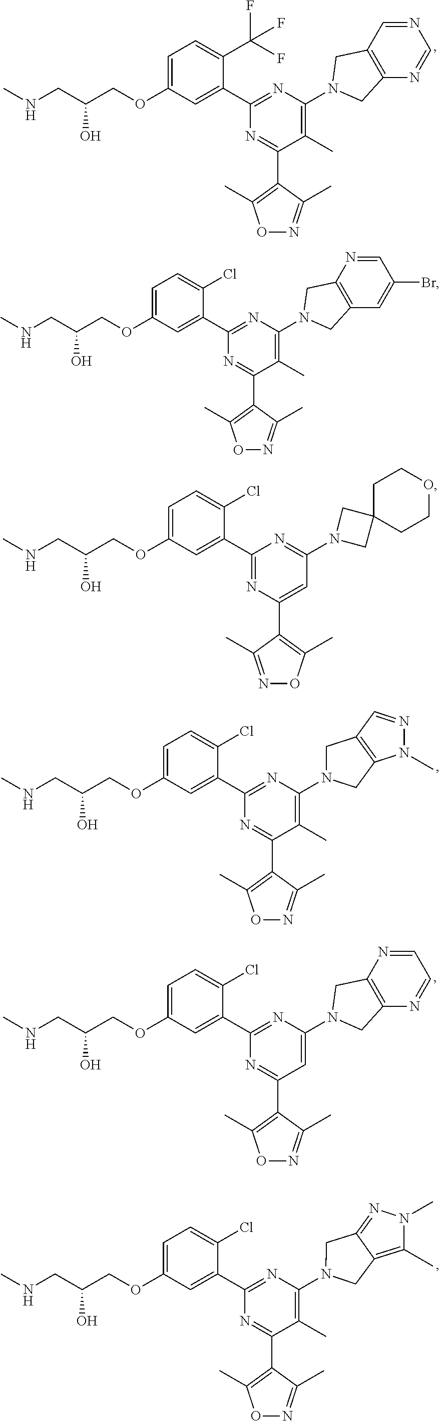

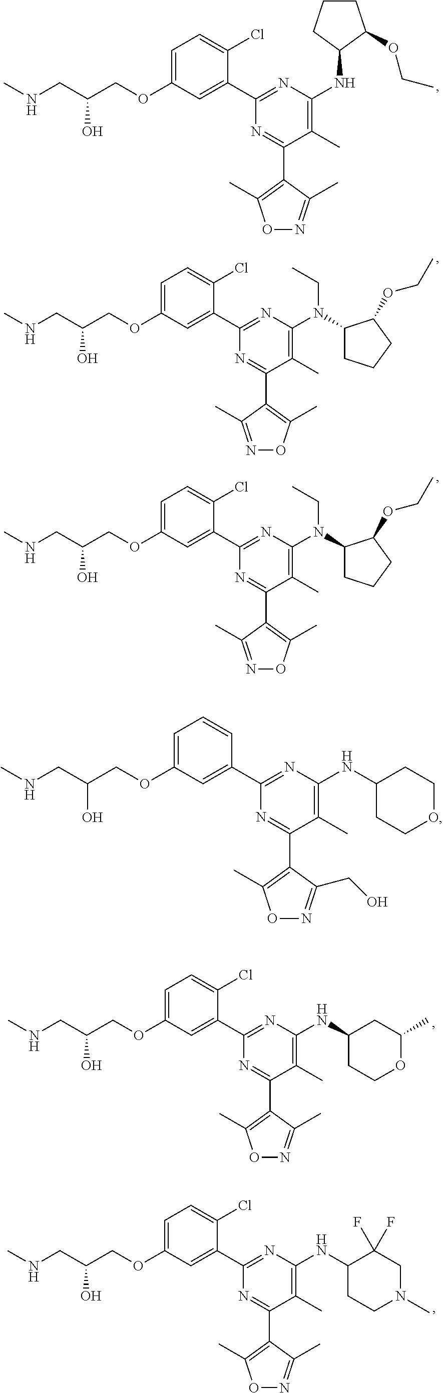

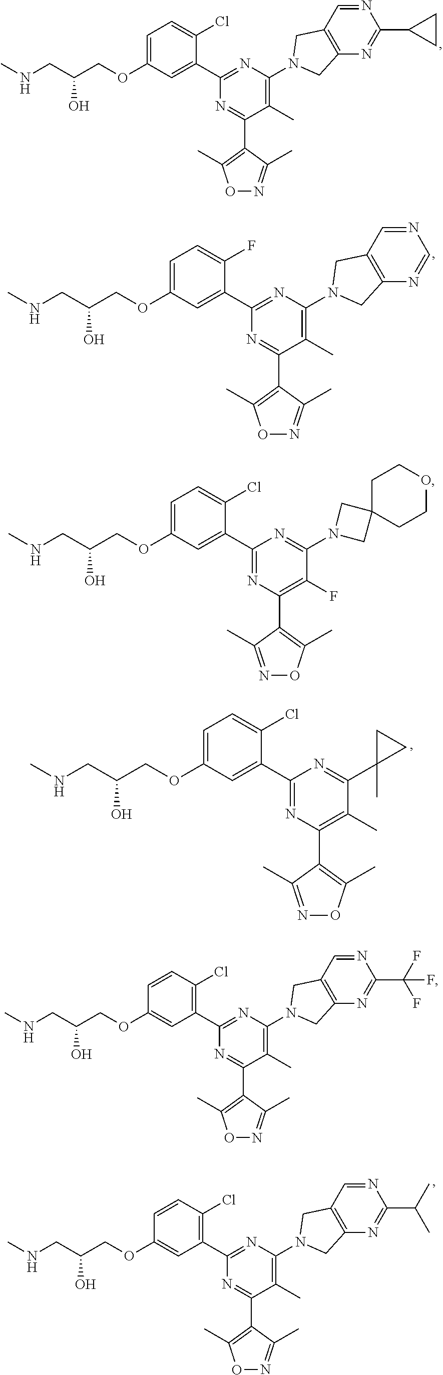

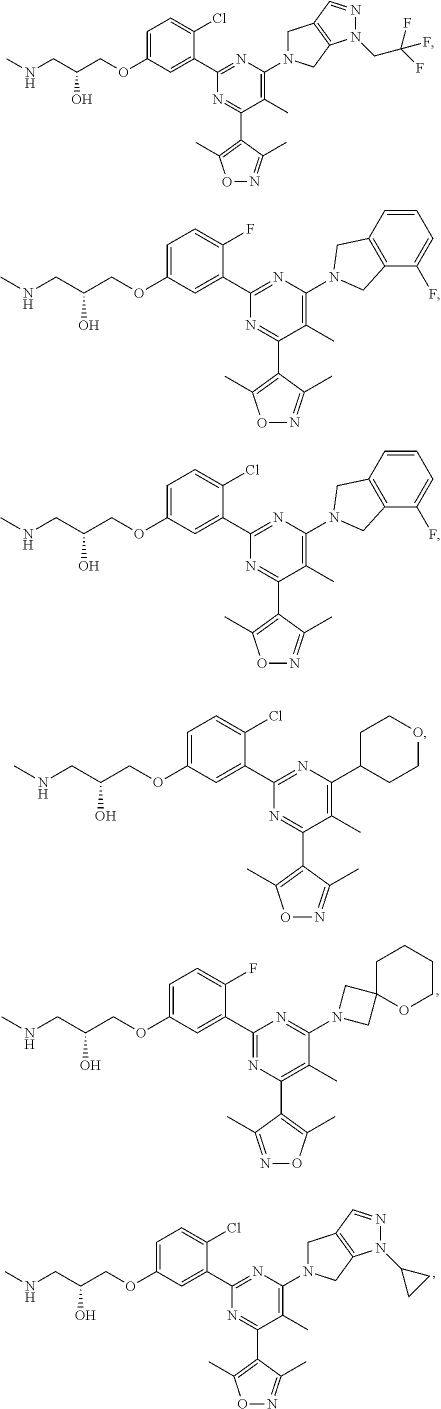 [SMILES: CNC[C@@H](O)COc1ccc(Cl)c(-c2nc(-c3c(C)noc3C)c(C)c(N3Cc4cnn(CC(F)(F)F)c4C3)n2)c1 ; CNC[C@@H](O)COc1ccc(F)c(-c2nc(-c3c(C)noc3C)c(C)c(N3Cc4cccc(F)c4C3)n2)c1 ; CNC[C@@H](O)COc1ccc(Cl)c(-c2nc(-c3c(C)noc3C)c(C)c(N3Cc4cccc(F)c4C3)n2)c1 ; CNC[C@@H](O)COc1ccc(Cl)c(-c2nc(-c3c(C)noc3C)c(C)c(C3CCOCC3)n2)c1 ; CNC[C@@H](O)COc1ccc(F)c(-c2nc(-c3c(C)noc3C)c(C)c(N3CC4(CCCCO4)C3)n2)c1 ; CNC[C@@H](O)COc1ccc(Cl)c(-c2nc(-c3c(C)noc3C)c(C)c(N3Cc4cnn(C5CC5)c4C3)n2)c1]

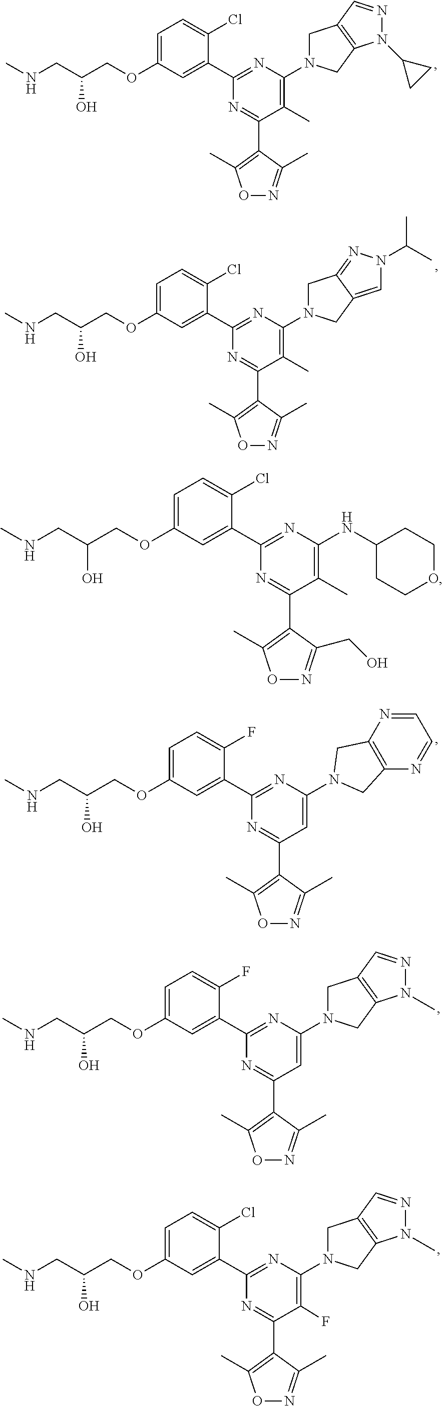

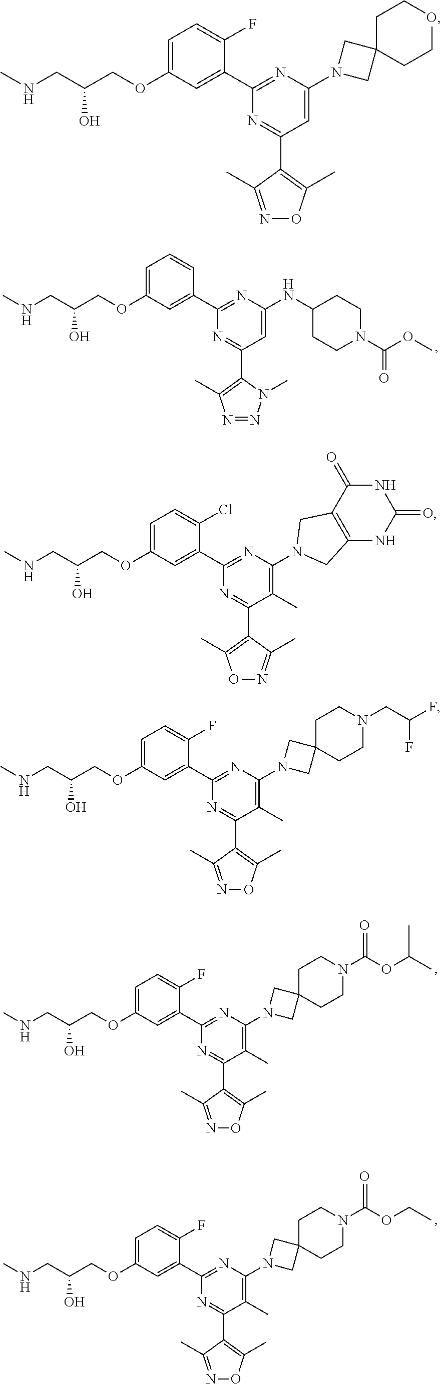

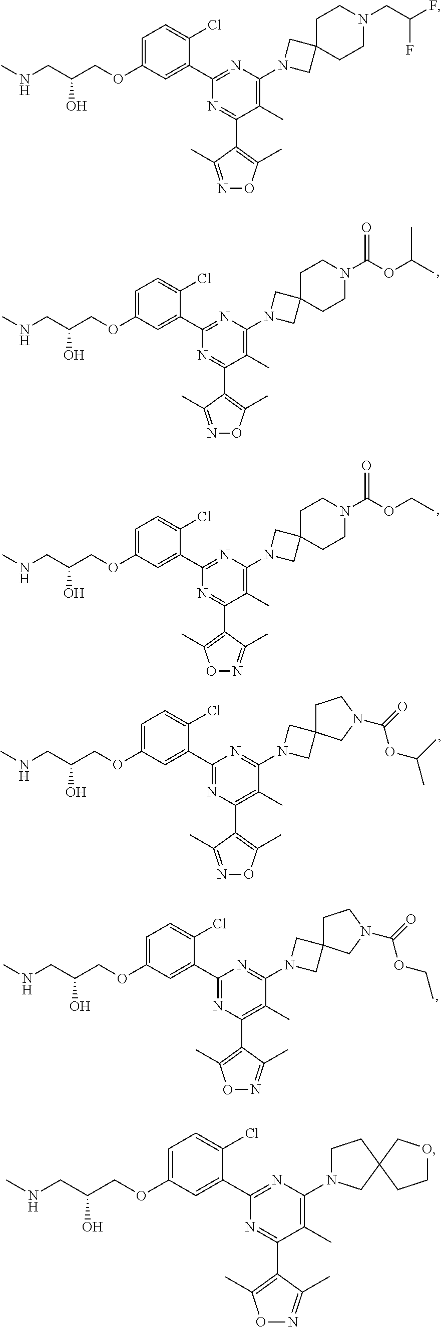

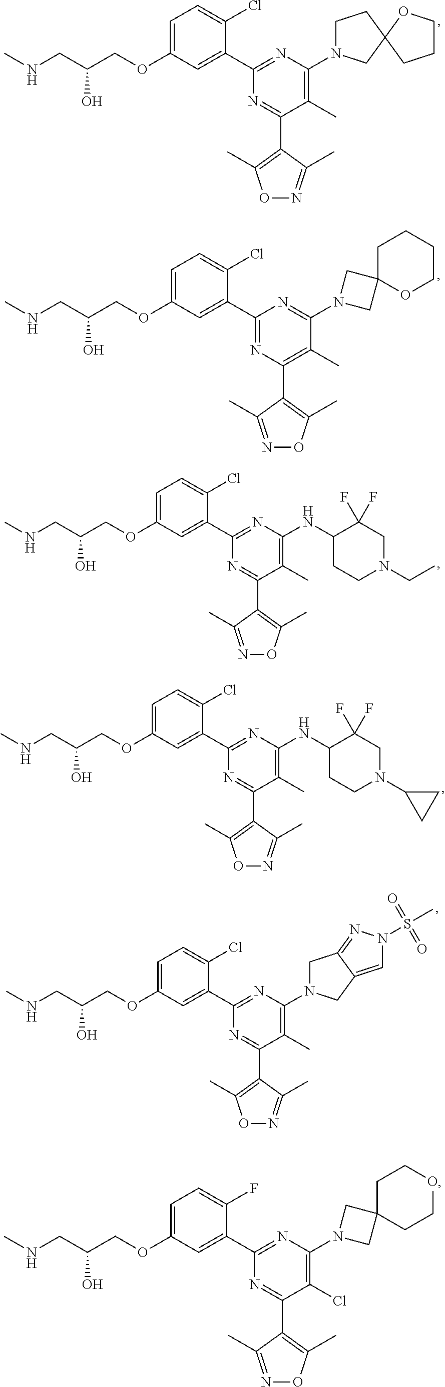

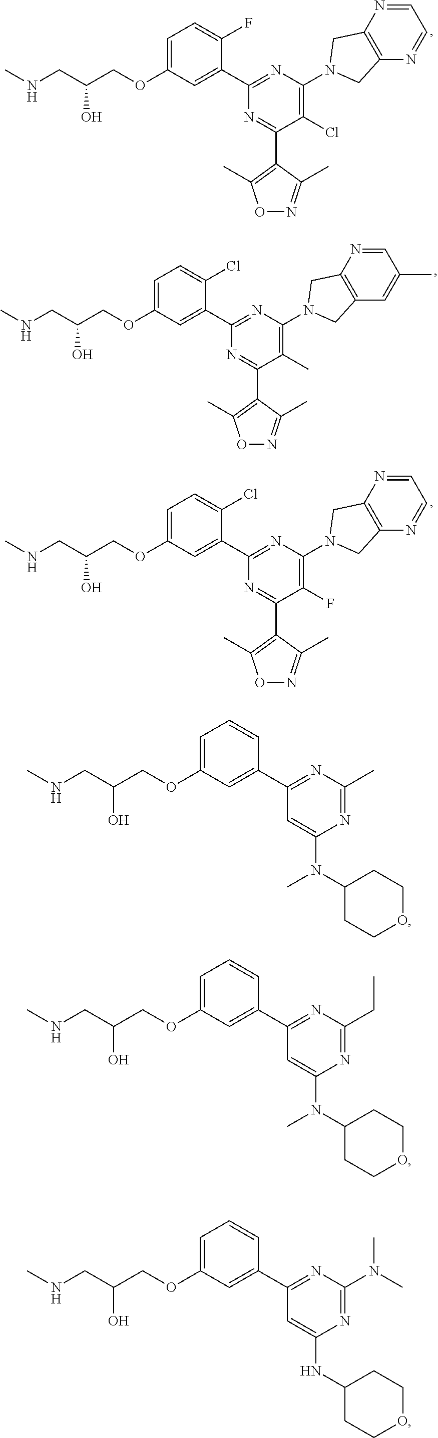

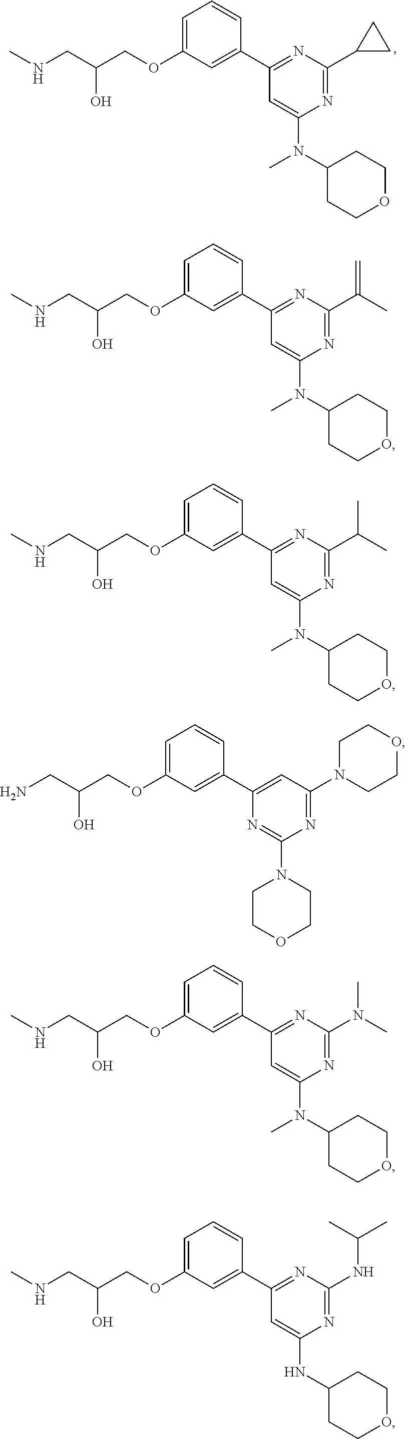

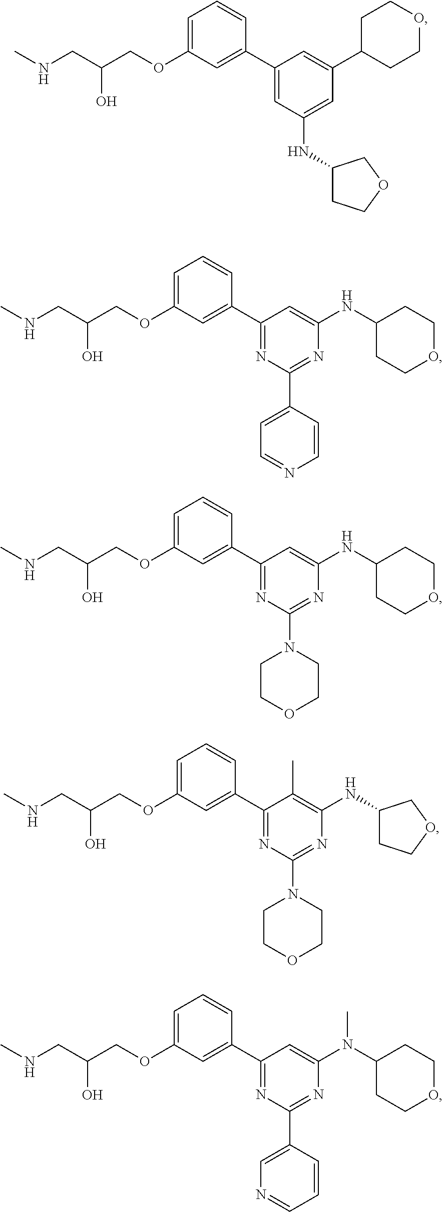 [SMILES: CNCC(O)COc1cccc(-c2cc(N[C@H]3CCOC3)cc(C3CCOCC3)c2)c1 ; CNCC(O)COc1cccc(-c2cc(NC3CCOCC3)nc(-c3ccncc3)n2)c1 ; CNCC(O)COc1cccc(-c2cc(NC3CCOCC3)nc(N3CCOCC3)n2)c1 ; CNCC(O)COc1cccc(-c2nc(N3CCOCC3)nc(N[C@H]3CCOC3)c2C)c1 ; CNCC(O)COc1cccc(-c2cc(N(C)C3CCOCC3)nc(-c3cccnc3)n2)c1]

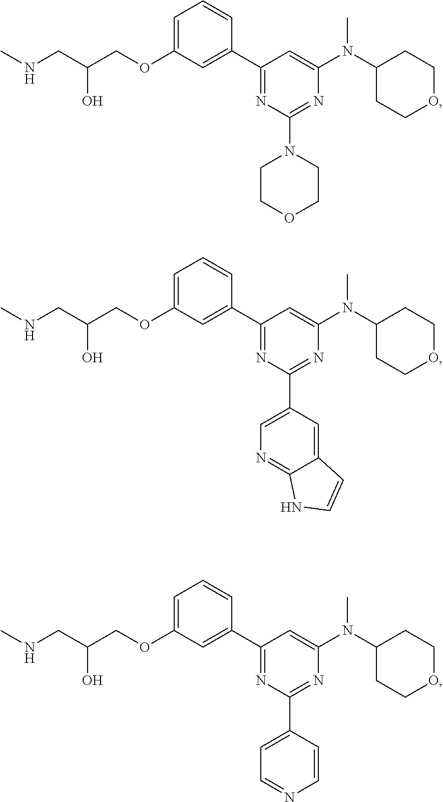 [SMILES: CNCC(O)COc1cccc(-c2cc(N(C)C3CCOCC3)nc(N3CCOCC3)n2)c1 ; CNCC(O)COc1cccc(-c2cc(N(C)C3CCOCC3)nc(-c3cnc4[nH]ccc4c3)n2)c1 ; CNCC(O)COc1cccc(-c2cc(N(C)C3CCOCC3)nc(-c3ccncc3)n2)c1]

and pharmaceutically acceptable salts thereof.

In certain embodiments, a provided compound inhibits CARM1. In certain embodiments, a provided compound inhibits wild-type CARM1. In certain embodiments, a provided compound inhibits a mutant CARM1. In certain embodiments, a provided compound inhibits CARM1, e.g., as measured in an assay described herein. In certain embodiments, the CARM1 is from a human. In certain embodiments, a provided compound inhibits CARM1 at an IC₅₀ less than or equal to 10 μM. In certain embodiments, a provided compound inhibits CARM1 at an IC₅₀ less than or equal to 1 μM. In certain embodiments, a provided compound inhibits CARM1 at an IC₅₀ less than or equal to 0.1 μM. In certain embodiments, a provided compound inhibits CARM1 in a cell at an EC₅₀ less than or equal to μM. In certain embodiments, a provided compound inhibits CARM1 in a cell at an EC₅₀ less than or equal to 1 μM. In certain embodiments, a provided compound inhibits CARM1 in a cell at an EC₅₀ less than or equal to 0.1 μM. In certain embodiments, a provided compound inhibits cell proliferation at an EC₅₀ less than or equal to 10 μM. In certain embodiments, a provided compound inhibits cell proliferation at an EC₅₀ less than or equal to 1 μM. In certain embodiments, a provided compound inhibits cell proliferation at an EC₅₀ less than or equal to 0.1 μM. In some embodiments, a provided compound is selective for CARM1 over other methyltransferases. In certain embodiments, a provided compound is at least about 10-fold selective, at least about 20-fold selective, at least about 30-fold selective, at least about 40-fold selective, at least about 50-fold selective, at least about 60-fold selective, at least about 70-fold selective, at least about 80-fold selective, at least about 90-fold selective, or at least about 100-fold selective for PRMT1 relative to one or more other methyltransferases.

It will be understood by one of ordinary skill in the art that the CARM1 can be wild-type CARM1, or any mutant or variant of CARM1.

The present disclosure provides pharmaceutical compositions comprising a compound described herein, e.g., a compound of Formula (I), or a pharmaceutically acceptable salt thereof, as described herein, and optionally a pharmaceutically acceptable excipient. It will be understood by one of ordinary skill in the art that the compounds described herein, or salts thereof, may be present in various forms, such as amorphous, hydrates, solvates, or polymorphs. In certain embodiments, a provided composition comprises two or more compounds described herein. In certain embodiments, a compound described herein, or a pharmaceutically acceptable salt thereof, is provided in an effective amount in the pharmaceutical composition. In certain embodiments, the effective amount is a therapeutically effective amount. In certain embodiments, the effective amount is an amount effective for inhibiting CARM1. In certain embodiments, the effective amount is an amount effective for treating a CARM1-mediated disorder. In certain embodiments, the effective amount is a prophylactically effective amount. In certain embodiments, the effective amount is an amount effective to prevent a CARM1-mediated disorder.

Pharmaceutically acceptable excipients include any and all solvents, diluents, or other liquid vehicles, dispersions, suspension aids, surface active agents, isotonic agents, thickening or emulsifying agents, preservatives, solid binders, lubricants, and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing a compound described herein (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the present disclosure will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60], sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the compounds described herein are mixed with solubilizing agents such as Cremophor™, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a provided compound may include ointments, pastes, creams, lotions, gels, powders, solutions, sprays, inhalants and/or patches. Generally, the active ingredient is admixed under sterile conditions with a pharmaceutically acceptable carrier and/or any desired preservatives and/or buffers as can be required. Additionally, the present disclosure encompasses the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A provided pharmaceutical composition can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A provided pharmaceutical composition can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A provided pharmaceutical composition can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, a compound described herein is administered one or more times per day, for multiple days. In some embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In certain embodiments, the additional therapeutically active agent is a compound of Formula (I). In certain embodiments, the additional therapeutically active agent is not a compound of Formula (I). In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of a provided compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the present disclosure are kits (e.g., pharmaceutical packs). The kits provided may comprise a provided pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of a provided pharmaceutical composition or compound. In some embodiments, a provided pharmaceutical composition or compound provided in the container and the second container are combined to form one unit dosage form. In some embodiments, a provided kits further includes instructions for use.

Compounds and compositions described herein are generally useful for the inhibition of CARM1. In some embodiments, the CARM1 is human CARM1. In some embodiments, methods of treating CARM1-mediated disorder in a subject are provided which comprise administering an effective amount of a compound described herein (e.g., a compound of Formula (I), or a pharmaceutically acceptable salt thereof), to a subject in need of treatment. In certain embodiments, the effective amount is a therapeutically effective amount. In certain embodiments, the effective amount is a prophylactically effective amount. In certain embodiments, the subject is suffering from a CARM1-mediated disorder. In certain embodiments, the subject is susceptible to a CARM1-mediated disorder.

As used herein, the term “CARM1-mediated disorder” means any disease, disorder, or other pathological condition in which CARM1 is known to play a role. Accordingly, in some embodiments, the present disclosure relates to treating or lessening the severity of one or more diseases in which CARM1 is known to play a role.

In some embodiments, the present disclosure provides a method of inhibiting CARM1 comprising contacting CARM1 with an effective amount of a compound described herein, e.g., a compound of Formula (I), or a pharmaceutically acceptable salt thereof. The CARM1 may be purified or crude, and may be present in a cell, tissue, or subject. Thus, such methods encompass both inhibition of in vitro and in vivo CARM1 activity. In certain embodiments, the method is an in vitro method, e.g., such as an assay method. It will be understood by one of ordinary skill in the art that inhibition of CARM1 does not necessarily require that all of the CARM1 be occupied by an inhibitor at once. Exemplary levels of inhibition of CARM1 include at least 10% inhibition, about 10% to about 25% inhibition, about 25% to about 50% inhibition, about 50% to about 75% inhibition, at least 50% inhibition, at least 75% inhibition, about 80% inhibition, about 90% inhibition, and greater than 90% inhibition.

In some embodiments, provided is a method of inhibiting CARM1 activity in a subject in need thereof comprising administering to the subject an effective amount of a compound described herein (e.g., a compound of Formula (I), or a pharmaceutically acceptable salt thereof, or a pharmaceutical composition thereof.

In certain embodiments, provided is a method of modulating gene expression or activity in a cell which comprises contacting a cell with an effective amount of a compound of Formula (I), or a pharmaceutically acceptable salt thereof. In certain embodiments, the cell in culture in vitro. In certain embodiments, the cell is in an animal, e.g., a human. In certain embodiments, the cell is in a subject in need of treatment.

In certain embodiments, provided is a method of modulating transcription in a cell which comprises contacting a cell with an effective amount of a compound of Formula (I), or a pharmaceutically acceptable salt thereof. In certain embodiments, the cell in culture in vitro. In certain embodiments, the cell is in an animal, e.g., a human. In certain embodiments, the cell is in a subject in need of treatment.

In certain embodiments, a method is provided of selecting a therapy for a subject having a disease associated with CARM1-mediated disorder or mutation comprising the steps of determining the presence of CARM1-mediated disorder or gene mutation in the CARM1 gene or and selecting, based on the presence of CARM1-mediated disorder a gene mutation in the CARM1 gene a therapy that includes the administration of a provided compound. In certain embodiments, the disease is cancer.

In certain embodiments, a method of treatment is provided for a subject in need thereof comprising the steps of determining the presence of CARM1-mediated disorder or a gene mutation in the CARM1 gene and treating the subject in need thereof, based on the presence of a CARM1-mediated disorder or gene mutation in the CARM1 gene with a therapy that includes the administration of a provided compound. In certain embodiments, the subject is a cancer patient.

In some embodiments, a compound provided herein is useful in treating a proliferative disorder, such as cancer. For example, while not being bound to any particular mechanism, protein arginine methylation by CARM1 is a modification that has been implicated in signal transduction, gene transcription, DNA repair and mRNA splicing, among others; and overexpression of CARM1 within these pathways is often associated with various cancers. Thus, compounds which inhibit the action of PRMTs, and specifically CARM1, as provided herein, are effective in the treatment of cancer.

In some embodiments, compounds provided herein are effective in treating cancer through the inhibition of CARM1. For example, CARM1 levels have been shown to be elevated in castration-resistant prostate cancer (CRPC) (e.g., see Di Lorenzo et al., Drugs (2010) 70:983-1000), as well as in aggressive breast tumors (Hong et al., Cancer 2004 101, 83-89; El Messaoudi et al., Proc. Natl. Acad. Sci. U.S.A 2006, 103, 13351-13356; Majumder et al., Prostate 2006 66, 1292-1301). Thus, in some embodiments, inhibitors of CARM1, as described herein, are useful in treating cancers associated with aberrant CARM1 activity, e.g., CARM1 overexpression or aberrant protein methylation. For example, aberrant CARM1 activity has been found in prostate cancer (e.g., see Hong et al., Cancer (2004), 101:83-89); plays a coactivator role in the dysragulation of beta-catenin activity in colorectal cancer (e.g., see Ou et al., Mol. Cancer Res. (2011) 9:660); and has been linked to estrogen signaling and estrogen related cancers such as breast cancer (see, e.g., Teyssiewr et al., Trends in Endocrinology and Metabolism (2010) 21:181-189). CARM1 has also been shown to affect estrogen receptor alpha (ER-alpha) dependent breast cancer cell differentiation and proliferation (Al-Dhaheri et al., Cancer Res. 2011 71, 2118-2128), thus in some aspects CARM1 inhibitors, as described herein, are useful in treating ERα-dependent breast cancer by inhibiting cell differentiation and proliferation. In another example, CARM1 has been shown to be recruited to the promoter of E2F1 (which encodes a cell cycle regulator) as a transcriptional co-activator (Frietze et al., Cancer Res. 2008 68, 301-306). Thus, CARM1-mediated upregulation of E2F1 expression may contribute to cancer progression and chemoresistance as increased abundance of E2F1 triggers invasion and metastasis by activating growth receptor signaling pathways, which in turn promote an antiapoptotic tumor environment (Engelmann and Piitzer, Cancer Res 2012 72; 571). Accordingly, in some embodiments, the inhibition of CARM1, e.g., by compounds provided herein, is useful in treating cancers associated with E2F1 upregulation, e.g., such as lung cancer (see, e.g., Eymin et al., Oncogene (2001) 20:1678-1687), and breast cancer (see, e.g., Brietz et al., Cancer Res. (2008) 68:301-306). Thus, without being bound by any particular mechanism, the inhibition of CARM1, e.g., by compounds described herein, is beneficial in the treatment of cancer. CARM1 overexpression has also been demonstrated to be elevated in 75% of colorectal cancers (Kim et al., BMC Cancer, 10, 197). It has been additionally been determined that depletion of CARM1 in WNT/β-catenin dysregulated colorectal cancer suppressed anchorage independent growth (Ou et al., Mol. Cancer. Res., 2011 9, 660-670). This, in some embodiments, the inhibition of CARM1, e.g. by compounds provided herein, is useful in colorectal cancer associated with elevated CARM1 expression or dysregulated WNT/β-catenin signaling.

In some embodiments, compounds described herein are useful for treating a cancer including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer, and vulvar cancer (e.g., Paget's disease of the vulva).

In certain embodiments, the cancer is a solid cancer. In certain embodiments, the cancer is a liquid cancer.

In certain embodiments, the cancer is breast cancer, prostate cancer, colorectal cancer, or a hematopoietic cancer (e.g., multiple myeloma).

CARM1 is also the most abundant PRMT expressed in skeletal muscle cells, and has been found to selectively control the pathways modulating glycogen metabolism, and associated AMPK (AMP-activated protein kinase) and p38 MAPK (mitogen-activated protein kinase) expression. See, e.g., Wang et al., Biochem (2012) 444:323-331. Thus, in some embodiments, inhibitors of CARM1, as described herein, are useful in treating metabolic disorders, e.g., for example skeletal muscle metabolic disorders, e.g., glycogen and glucose metabolic disorders. Exemplary skeletal muscle metabolic disorders include, but are not limited to, Acid Maltase Deficiency (Glycogenosis type 2; Pompe disease), Debrancher deficiency (Glycogenosis type 3), Phosphorylase deficiency (McArdle's; GSD 5), X-linked syndrome (GSD9D), Autosomal recessive syndrome (GSD9B), Tarui's disease (Glycogen storage disease VII; GSD 7), Phosphoglycerate Mutase deficiency (Glycogen storage disease X; GSDX; GSD 10), Lactate dehydrogenase A deficiency (GSD 11), Branching enzyme deficiency (GSD 4), Aldolase A (muscle) deficiency, β-Enolase deficiency, Triosephosphate isomerase (TIM) deficiency, Lafora's disease (Progressive myoclonic epilepsy 2), Glycogen storage disease (Muscle, Type 0, Phosphoglucomutase 1 Deficiency (GSD 14)), and Glycogenin Deficiency (GSD 15).

### EXAMPLES

In order that the invention described herein may be more fully understood, the following examples are set forth. It should be understood that these examples are for illustrative purposes only and are not to be construed as limiting this invention in any manner.

### Synthetic Methods

The synthesis of an exemplary set of compounds of Formula (I) is provided below. These compounds are also provided in Table 1.

### Example 1. Preparation of methyl 2-(2-(5-((R)-2-hydroxy-3-(methylamino)propoxy)phenyl)-6-(1,4-dimethyl-1H-pyrazol-5-yl)-5-methylpyrimidin-4-yl)-2,7-diazaspiro[3.5] nonane-7-carboxylate

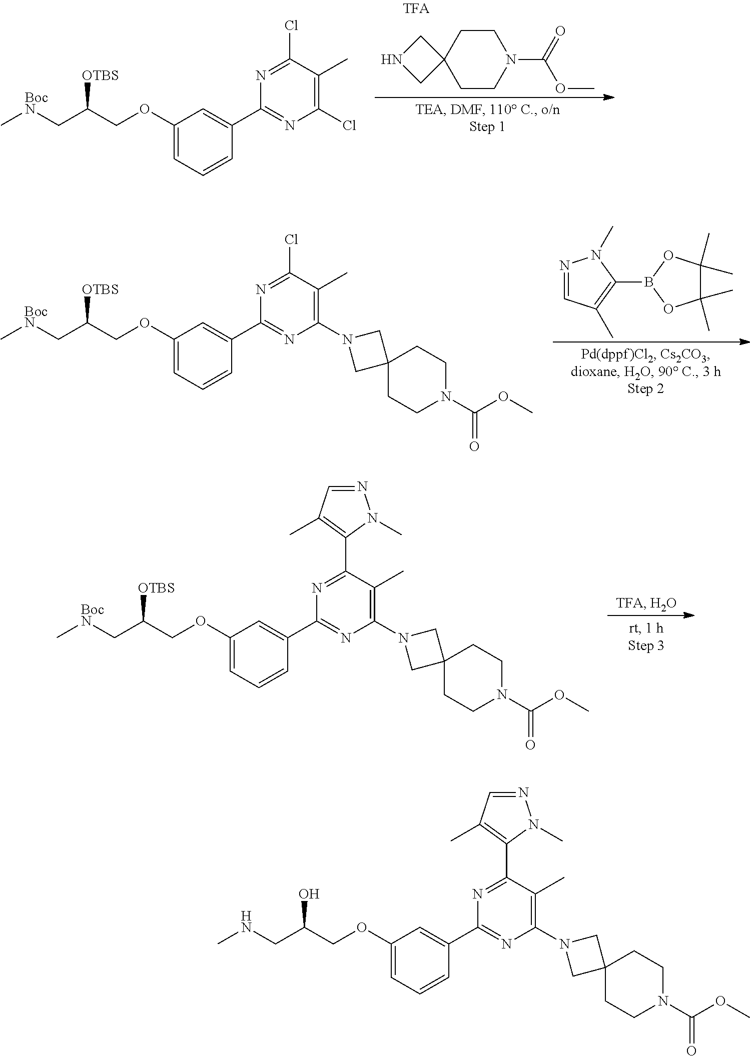

### Step 1: methyl 2-(2-(3-((R)-3-(tert-butoxycarbonyl(methyl)amino)-2-(tert-butyldimethylsilyloxy)propoxy)phenyl)-6-chloro-5-methylpyrimidin-4-yl)-2,7-diazaspiro[3.5]nonane-7-carboxylate

To a solution of (R)-tert-butyl 2-(tert-butyldimethylsilyloxy)-3-(3-(4,6-dichloro-5-methylpyrimidin-2-yl)phenoxy)propyl(methyl)carbamate (1 g, 1.8 mmol) in DMF (20 mL) was added methyl 2,7-diazaspiro[3.5]nonane-7-carboxylate TFA salt (2.15 g, 7.2 mmol) and triethylamine (909 mg, 9 mmol) at room temperature. The reaction mixture was heated at 110° C. for 16 h, cooled down to room temperature, diluted with EtOAc (120 mL) and then washed with water (80 mL×2) and brine (80 mL). The organic layer was dried over Na₂SO₄, filtered and concentrated in vacuo to render a residue which was purified by column chromatography over silicagel to give methyl 2-(2-(3-((R)-3-(tert-butoxycarbonyl(methyl)amino)-2-(tert-butyldimethylsilyloxy) propoxy)phenyl)-6-chloro-5-methylpyrimidin-4-yl)-2,7-diazaspiro[3.5] nonane-7-carboxylate as a yellow solid (1.2 g, 95% yield). ESI-LCMS (m/z): 704.3 found for [M+1]⁺.

### Step 2: methyl 2-(2-(3-((R)-3-(tert-butoxycarbonyl(methyl)amino)-2-(tert-butyldimethylsilyloxy)propoxy)phenyl)-6-(1,4-dimethyl-1H-pyrazol-5-yl)-5-methyl pyrimidin-4-yl)-2,7-diazaspiro[3.5]nonane-7-carboxylate

To a solution of methyl 2-(2-(3-((R)-3-(tert-butoxycarbonyl(methyl)amino)-2-(tert-butyldimethylsilyloxy)propoxy)phenyl)-6-chloro-5-methylpyrimidin-4-yl)-2,7-diaza-spiro[3.5] nonane-7-carboxylate (160 mg, 0.23 mmol) in degassed dioxane:H₂O (5:1, 6 mL) was added 1,4-dimethyl-5-(4,4,5,5-tetramethyl-1,3,2-dioxaborolan-2-yl)-1H-pyrazole (78 mg, 0.35 mmol), Pd(dppf)Cl₂ (16 mg, 0.02 mmol) and Na₂CO₃ (73 mg, 0.69 mmol) at room temperature. The system was purged with N₂ and the mixture was stirred at 90° C. for 3 h. After being cooled down to room temperature the mixture was filtered through a pad of celite. The filtrate was concentrated and the resulting residue was purified by preparative-TLC on silicagel eluting with petroleum ether/EtOAc=2/1 to give methyl 2-(2-(3-((R)-3-(tert-butoxycarbonyl (methyl)amino)-2-(tert-butyldimethylsilyloxy)propoxy) phenyl)-6-(1,4-dimethyl-1H-pyrazol-5-yl)-5-methylpyrimidin-4-yl)-2,7-diazaspiro[3.5]nonane-7-carboxylate (130 mg, 74% yield) as a white solid. ESI-LCMS (m/z): 764.1 found for [M+1]⁺.

### Step 3: methyl 2-(2-(5-((R)-2-hydroxy-3-(methylamino)propoxy) phenyl)-6-(1,4-dimethyl-1H-pyrazol-5-yl)-5-methylpyrimidin-4-yl)-2,7-diazaspiro[3.5] nonane-7-carboxylate

A solution of methyl 2-(2-(3-((R)-3-(tert-butoxycarbonyl(methyl)amino)-2-(tert-butyldimethyl-silyloxy)propoxy)phenyl)-6-(1,4-dimethyl-1H-pyrazol-5-yl)-5-methylpyrimidin-4-yl)-2,7-diazaspiro[3.5]nonane-7-carboxylate (130 mg, 0.17 mmol) in 90% TFA (2 mL), was stirred at room-temperature for 1 h. The solvent was then removed in vacuo and the resulting residue was dissolved in MeOH (5 mL). The solution was adjusted pH 7-8 with aqueous K₂CO₃ and concentrated. The residue was purified by preparative-HPLC to give methyl 2-(6-(1,4-dimethyl-1H-pyrazol-5-yl)-2-(3-((R)-2-hydroxy-3-(methylamino) propoxy) phenyl)-5-methylpyrimidin-4-yl)-2,7-diazaspiro[3.5]nonane-7-carboxylate as a white solid (20 mg, 21%). ESI-LCMS (m/z): 550.2 found for [M+1]+; ¹HNMR (400 MHz, CD₃OD) δ ppm: 7.92-7.87 (m, 2H), 7.38 (s, 1H), 7.32 (t, J=7.6 Hz, 1H), 7.03 (dd, J=2.0 and 7.6 Hz, 1H), 4.23-4.12 (m, 4H), 4.10-4.07 (m, 1H), 4.03-3.98 (m, 2H), 3.73 (s, 3H), 3.67 (s, 3H), 3.55-3.45 (m, 4H), 2.86-2.70 (m, 2H), 2.44 (s, 3H), 2.08 (s, 3H), 1.98 (s, 3H), 1.86-1.78 (m, 4H).

### Example 2. Preparation of (2R)-1-(4-chloro-3-(4-(4-chloro-1-methyl-1H-pyrazol-5-yl)-5-methyl-6-(5H-pyrrolo[3,4-b]pyridin-6(7H)-yl)pyrimidin-2-yl)phenoxy)-3-(methyl-amino)propan-2-ol

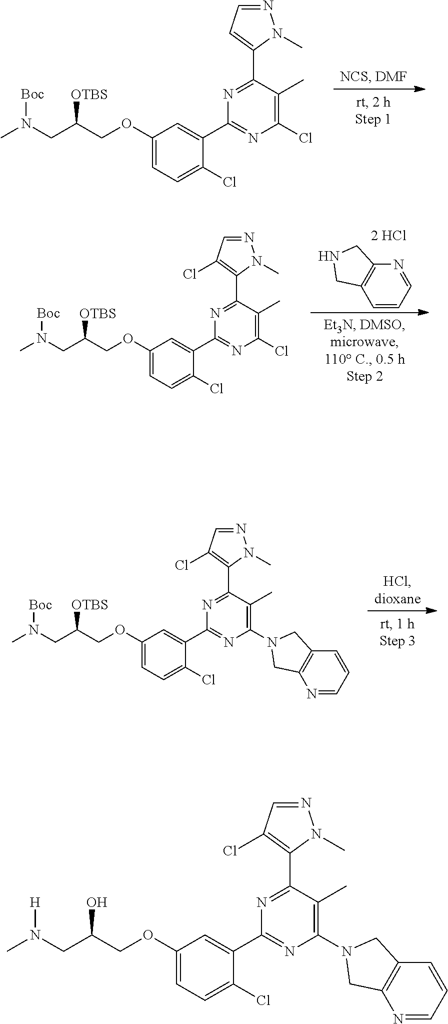 [SMILES: Cc1c(Cl)nc(-c2cc(OC[C@@H](CN(C)C(=O)OC(C)(C)C)O[Si](C)(C)C(C)(C)C)ccc2Cl)nc1-c1ccnn1C ; Cc1c(Cl)nc(-c2cc(OC[C@@H](CN(C)C(=O)OC(C)(C)C)O[Si](C)(C)C(C)(C)C)ccc2Cl)nc1-c1c(Cl)cnn1C ; c1cnc2c(c1)CNC2 ; Cc1c(-c2c(Cl)cnn2C)nc(-c2cc(OC[C@@H](CN(C)C(=O)OC(C)(C)C)O[Si](C)(C)C(C)(C)C)ccc2Cl)nc1N1Cc2cccnc2C1 ; CNC[C@@H](O)COc1ccc(Cl)c(-c2nc(-c3c(Cl)cnn3C)c(C)c(N3Cc4cccnc4C3)n2)c1]

### Step 1: tert-butyl (2R)-2-(tert-butyldimethylsilyloxy)-3-(4-chloro-3-(4-chloro-6-(4-chloro-1-methyl-1H-pyrazol-5-yl)-5-methylpyrimidin-2-yl) phenoxy)propyl(methyl)carbamate

A solution of (R)-tert-butyl 2-(tert-butyldimethylsilyloxy)-3-(4-chloro-3-(4-chloro-5-methyl-6-(1-methyl-1H-pyrazol-5-yl)pyrimidin-2-yl)phenoxy)propyl(methyl) carbamate (120 mg, 0.19 mmol) and NCS (45 mg, 0.34 mmol) in DMF (2 ml) was stirred at room temperature for 2 h; the mixture was then diluted with water (10 mL) and extracted with EtOAc (10 mL×3). The combined organic layers were washed with water (10 mL) and brine (10 mL), dried over Na₂SO₄, filtered and concentrated to give tert-butyl (2R)-2-(tert-butyl-dimethylsilyloxy)-3-(4-chloro-3-(4-chloro-6-(4-chloro-1-methyl-1H-pyrazol-5-yl)-5-methylpyrimidin-2-yl)phenoxy)propyl (methyl)carbamate (120 mg, 94% yield) as white solid. ESI-LCMS (m/z): 670 found for [M+1]⁺.

### Step 2: tert-butyl (R)-2-(tert-butyldimethylsilyloxy)-3-(4-chloro-3-(4-(4-chloro-1-methyl-1H-pyrazol-5-yl)-5-methyl-6-(5H-pyrrolo[3,4-b]pyridin-6(7H)-yl) pyrimidin-2-yl)phenoxy)propyl(methyl)carbamate

A reaction pressure vessel was charged with a mixture of tert-butyl (R)-2-(tert-butyldimethylsilyloxy)-3-(4-chloro-3-(4-chloro-6-(4-chloro-1-methyl-1H-pyrazol-5-yl)-5-methylpyrimidin-2-yl)phenoxy)propyl (methyl)carbamate (90 mg, 0.13 mmol); 6,7-dihydro-5H-pyrrolo[3,4-b]pyridine dihydrochloride (or any other suitably substituted primary or secondary amine, 0.15 mmol), triethylamine (30 mg, 0.3 mmol) and n-BuOH (2 mL). The vessel was capped, placed in a microwave reactor and irradiated for 30 min. at external temperature of 110° C. After being cooled down to room temperature, the mixture was diluted with water (20 mL) and extracted with EtOAc (20 mL×3). The combined organic layers were washed with water (20 mL) and brine (20 mL), dried over Na₂SO₄, filtered and concentrated to give tert-butyl (R)-2-(tert-butyldimethylsilyloxy)-3-(4-chloro-3-(4-(4-chloro-1-methyl-1H-pyrazol-5-yl)-5-methyl-6-(5H-pyrrolo[3,4-b]pyridin-6(7H)-yl)pyrimidin-2-yl)phenoxy)propyl(methyl)carbamate as a yellow solid (120 mg, crude), which was used directly into next step without further purification. ESI-LCMS (m/z): 754.0 found for [M+1]⁺.

### Step 3: (2R)-1-(4-chloro-3-(4-(4-chloro-1-methyl-1H-pyrazol-5-yl)-5-methyl-6-(5H-pyrrolo[3,4-b]pyridin-6(7H)-yl)pyrimidin-2-yl)phenoxy)-3-(methyl-amino)propan-2-ol

A solution of tert-butyl (R)-2-(tert-butyldimethylsilyloxy)-3-(4-chloro-3-(4-(4-chloro-1-methyl-1H-pyrazol-5-yl)-5-methyl-6-(5H-pyrrolo[3,4-b]pyridin-6(7H)-yl)pyrimidin-2-yl) phenoxy) propyl(methyl)carbamate (120 mg, crude, from step 2) in a 4N HCl solution in dioxane (6 mL), was stirred at room temperature for 1 h. The solvent was then removed in vacuo and the resulting residue was purified by preparative HPLC to give (2R)-1-(4-chloro-3-(4-(4-chloro-1-methyl-1H-pyrazol-5-yl)-5-methyl-6-(5H-pyrrolo[3,4-b]pyridin-6(7H)-yl)pyrimidin-2-yl) phenoxy)-3-(methylamino)propan-2-ol as a white solid (22 mg, 31% yield for 2 steps). ESI-LCMS (m/z): 540.2 found for [M+H]+; ¹HNMR (400 MHz, CD₃OD) δ ppm: 8.50 (d, J=4.8 Hz, 1H), 7.88 (d, J=7.6 Hz, 1H), 7.61 (s, 1H), 7.44-7.38 (m, 2H), 7.35 (d, J=3.2 Hz, 1H), 7.05 (dd, J=2.8 and 8.8 Hz, 1H), 5.44 (t, J=14.8 Hz, 2H), 5.23 (t, J=16.8 Hz, 2H), 4.15-4.08 (m, 1H), 4.06-3.98 (m, 2H), 3.89 (s, 3H), 2.86-2.73 (m, 2H), 2.51 (s, 3H), 2.47 (s, 3H).

### Example 3. Preparation of (2R)-1-(3-(4-(4-bromo-1-methyl-1H-pyrazol-5-yl)-5-methyl-6-(5H-pyrrolo[3,4-b]pyridin-6(7H)-yl)pyrimidin-2-yl)-4-chlorophenoxy)-3-(methyl-amino)propan-2-ol

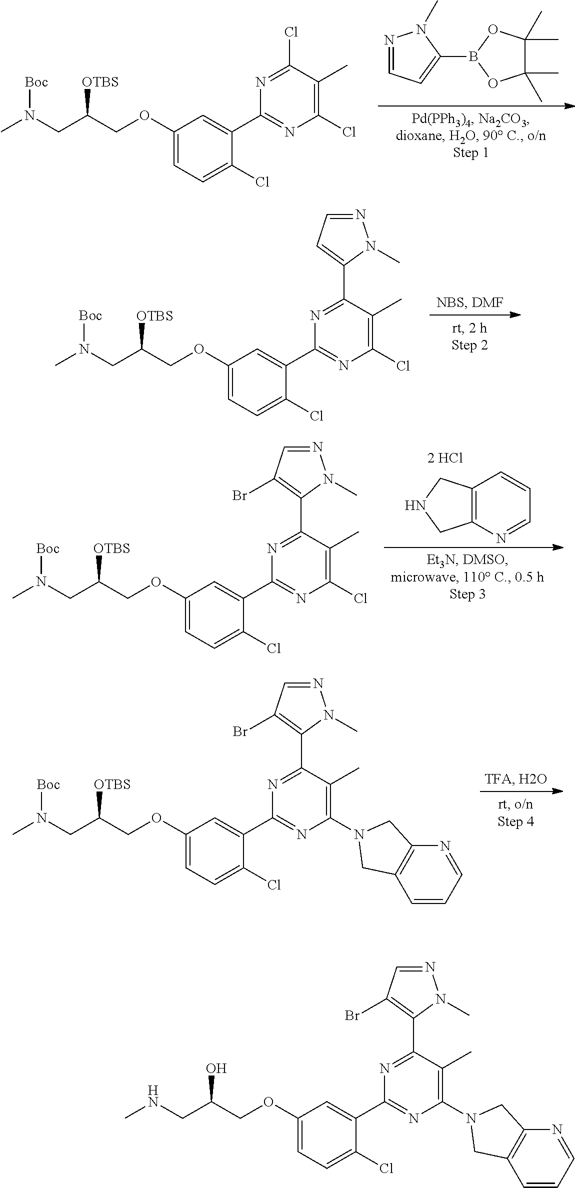

### Step 1: (R)-tert-butyl 2-(tert-butyldimethylsilyloxy)-3-(4-chloro-3-(4-chloro-5-methyl-6-(1-methyl-1H-pyrazol-5-yl)pyrimidin-2-yl)phenoxy)propyl (methyl)carbamate

To a solution of (R)-tert-butyl 2-(tert-butyldimethylsilyloxy)-3-(4-chloro-3-(4,6-di-chloro-5-methylpyrimidin-2-yl)phenoxy)propyl(methyl)carbamate (1.0 g, 1.7 mmol) in degassed dioxane and H₂O (3/1, 24 mL) was added Na₂CO₃ (541 mg, 5.1 mmol), Pd(PPh₃)₄(98 mg, 0.08 mmol) and 1-methyl-5-(4,4,5,5-tetramethyl-1,3,2-dioxa-borolan-2-yl)-1H-pyrazole (707 mg, 3.4 mmol) at room temperature. The system was purged with N₂ and the mixture was stirred at 90° C. for 16 h. After being cooled down to room temperature the solvent was removed in vacuo. The residue was diluted with water (30 mL) and extracted with EtOAc (100 mL×2). The combined organic layers were washed with water (50 mL) and brine (50 mL), dried over Na₂SO₄, filtered and concentrated. The residue was purified by column chromatography over silicagel (petroleum ether/EtOAc=4/1) to give (R)-tert-butyl 2-(tert-butyldimethylsilyloxy)-3-(4-chloro-3-(4-chloro-5-methyl-6-(1-methyl-1H-pyrazol-5-yl)pyrimidin-2-yl)phenoxy)propyl(methyl)carbamate (420 mg, 39% yield). ESI-LCMS (m/z): 658.2 found for [M+23]⁺.

### Step 2: tert-butyl (2R)-3-(3-(4-(4-bromo-1-methyl-1H-pyrazol-5-yl)-6-chloro-5-methylpyrimidin-2-yl)-4-chlorophenoxy)-2-(tert-butyldimethyl-silyloxy)propyl(methyl)carbamate

A solution of (R)-tert-butyl 2-(tert-butyldimethylsilyloxy)-3-(4-chloro-3-(4-chloro-5-methyl-6-(1-methyl-1H-pyrazol-5-yl)pyrimidin-2-yl)phenoxy)propyl(methyl) carbamate (120 mg, 0.19 mmol) and NBS (50 mg, 0.28 mmol) in DMF (3 ml) was stirred at room temperature for 2 h. After the reaction was complete the mixture was diluted with water (10 mL) and extracted with EtOAc (20 mL×3). The combined organic layers were washed with water (20 mL) and brine (20 mL), dried over Na₂SO₄, filtered and concentrated to give tert-butyl (2R)-3-(3-(4-(4-bromo-1-methyl-1H-pyrazol-5-yl)-6-chloro-5-methylpyrimidin-2-yl)-4-chloro-phenoxy)-2-(tert-butyl dimethylsilyloxy)propyl(methyl)carbamate (120 mg, 88% yield). ESI-LCMS (m/z): 736.1 found for [M+23]⁺.

### Step 3: tert-butyl (2R)-3-(3-(4-(4-bromo-1-methyl-1H-pyrazol-5-yl)-5-methyl-6-(5H-pyrrolo[3,4-b]pyridin-6(7H)-yl)pyrimidin-2-yl)-4-chlorophenoxy)-2-(tert-butyldimethylsilyloxy)propyl(methyl)carbamate

A reaction pressure vessel was charged with a mixture of tert-butyl (2R)-3-(3-(4-(4-bromo-1-methyl-1H-pyrazol-5-yl)-6-chloro-5-methylpyrimidin-2-yl)-4-chlorophenoxy)-2-(tert-butyl dimethylsilyloxy)-propyl(methyl)carbamate (120 mg, 0.17 mmol); 6,7-dihydro-5H-pyrrolo[3,4-b]pyridine dihydrochloride (or any other suitably substituted primary or secondary amine, 0.34 mmol), triethylamine (0.5 mL, 3.5 mmol) and DMSO (3 mL). The vessel was capped, placed in a microwave reactor and irradiated for 30 min. at external temperature of 110° C. After being cooled down to room temperature, the mixture was diluted with water (15 mL) and extracted with EtOAc (20 mL×3). The combined organic layers were washed with water (20 mL) and brine (20 mL), dried over Na₂SO₄, filtered and concentrated. The residue was purified by column chromatography over silicagel to give tert-butyl (2R)-3-(3-(4-(4-bromo-1-methyl-1H-pyrazol-5-yl)-5-methyl-6-(5H-pyrrolo[3,4-b]pyridin-6(7H)-yl)pyrimidin-2-yl)-4-chlorophenoxy)-2-(tert-butyldimethyl-silyloxy)propyl(methyl)carbamate (120 mg, 89% yield) as white solid. ESI-LCMS (m/z): 798.2 found for [M+H]⁺.

### Step 4: (2R)-1-(3-(4-(4-bromo-1-methyl-1H-pyrazol-5-yl)-5-methyl-6-(5H-pyrrolo[3,4-b]pyridin-6(7H)-yl)pyrimidin-2-yl)-4-chlorophenoxy)-3-(methylamino)propan-2-ol

A solution of tert-butyl (2R)-3-(3-(4-(4-bromo-1-methyl-1H-pyrazol-5-yl)-5-methyl-6-(5H-pyrrolo[3,4-b]pyridin-6(7H)-yl)pyrimidin-2-yl)-4-chlorophenoxy)-2-(tert-butyldi-methylsilyl oxy)propyl(methyl)carbamate (120 mg, 0.15 mmol) in 90% TFA (2 mL) was stirred at room temperature for 16 h; concentrated in vacuo and the residue was dissolved in MeOH (2 ml); the resulting solution was adjusted to pH 7-8 with aqueous K₂CO₃ solution, filtered and the filtrate was concentrated again. The residue was purified by preparative-HPLC to give (2R)-1-(3-(4-(4-bromo-1-methyl-1H-pyrazol-5-yl)-5-methyl-6-(5H-pyrrolo[3,4-b]pyridin-6(7H)-yl)pyrimidin-2-yl)-4-chlorophenoxy)-3-(methylamino)propan-2-ol (26 mg, 30% yield) as white solid. ESI-LCMS (m/z): 584.1 found for [M+H]+; ¹HNMR (400 MHz, CD₃OD) δ ppm:: 8.50 (d, J=5.2 Hz, 1H), 7.89 (d, J=7.6 Hz, 1H), 7.64 (s, 1H), 7.45-7.39 (m, 2H), 7.35 (d, J=3.2 Hz, 1H), 7.07 (dd, J=2.8 and 8.8 Hz, 1H), 5.43 (t, J=14.8 Hz, 2H), 5.24 (t, J=16.8 Hz, 2H), 4.14-4.10 (m, 1H), 4.05-4.01 (m, 2H), 3.89 (s, 3H), 2.90-2.75 (m, 2H), 2.50 (s, 3H), 2.48 (s, 3H).

### Example 4. Preparation of (2R)-1-(4-chloro-3-(4-(4-iodo-1-methyl-1H-pyrazol-5-yl)-5-methyl-6-(5H-pyrrolo[3,4-b]pyridin-6(7H)-yl)pyrimidin-2-yl)phenoxy)-3-(methyl-amino)propan-2-ol

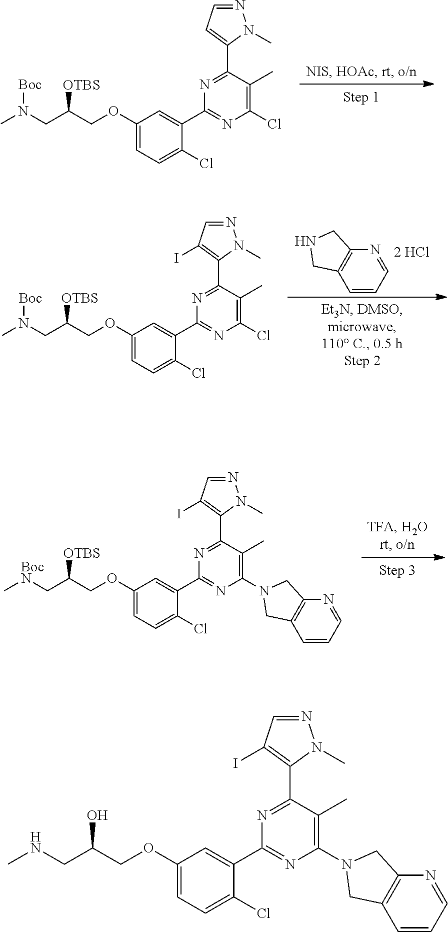 [SMILES: Cc1c(Cl)nc(-c2cc(OC[C@@H](CN(C)C(=O)OC(C)(C)C)O[Si](C)(C)C(C)(C)C)ccc2Cl)nc1-c1ccnn1C ; Cc1c(Cl)nc(-c2cc(OC[C@@H](CN(C)C(=O)OC(C)(C)C)O[Si](C)(C)C(C)(C)C)ccc2Cl)nc1-c1c(I)cnn1C ; c1cnc2c(c1)CNC2 ; Cc1c(-c2c(I)cnn2C)nc(-c2cc(OC[C@@H](CN(C)C(=O)OC(C)(C)C)O[Si](C)(C)C(C)(C)C)ccc2Cl)nc1N1Cc2cccnc2C1 ; CNC[C@@H](O)COc1ccc(Cl)c(-c2nc(-c3c(I)cnn3C)c(C)c(N3Cc4cccnc4C3)n2)c1]

### Step 1: tert-butyl (2R)-2-(tert-butyldimethylsilyloxy)-3-(4-chloro-3-(4-chloro-6-(4-iodo-1-methyl-1H-pyrazol-5-yl)-5-methylpyrimidin-2-yl)phenoxy)propyl(methyl)carbamate

To a solution of (R)-tert-butyl 2-(tert-butyldimethylsilyloxy)-3-(4-chloro-3-(4-chloro-5-methyl-6-(1-methyl-1H-pyrazol-5-yl)pyrimidin-2-yl)phenoxy)propyl(methyl) carbamate (220 mg, 0.35 mmol) in HOAc (3 ml) was added NIS (117 mg, 0.52 mmol). The mixture was stirred at room temperature for 16 h., diluted with water (10 mL) and extracted with EtOAc (20 mL×3). The combined organic layers were washed with water (20 mL) and brine (20 mL), dried over Na₂SO₄, filtered and concentrated to give tert-butyl (2R)-2-(tert-butyldimethylsilyloxy)-3-(4-chloro-3-(4-chloro-6-(4-iodo-1-methyl-1H-pyrazol-5-yl)-5-methylpyrimidin-2-yl)phenoxy)propyl(methyl)carbamate (160 mg, 60% yield). ESI-LCMS (m/z): 784.0 found for [M+Na]⁺.

### Step 2: tert-butyl (2R)-2-(tert-butyldimethylsilyloxy)-3-(4-chloro-3-(4-(4-iodo-1-methyl-1H-pyrazol-5-yl)-5-methyl-6-(5H-pyrrolo[3,4-b]pyridin-6(7H)-yl)pyrimidin-2-yl)phenoxy)propyl(methyl)carbamate

A reaction pressure vessel was charged with a mixture of tert-butyl (2R)-2-(tert-butyldimethylsilyloxy)-3-(4-chloro-3-(4-chloro-6-(4-iodo-1-methyl-1H-pyrazol-5-yl)-5-methylpyrimidin-2-yl)phenoxy)propyl(methyl)carbamate (160 mg, 0.2 mmol); 6,7-dihydro-5H-pyrrolo[3,4-b]pyridine dihydrochloride (or any other suitably substituted primary or secondary amine, 0.4 mmol), triethylamine (0.5 mL, 3.5 mmol) and DMSO (3 mL). The vessel was capped, placed in a microwave reactor and irradiated for 30 min. at external temperature of 110° C. After being cooled down to room temperature, the mixture was diluted with water (15 mL) and extracted with EtOAc (20 mL×3). The combined organic layers were dried over Na₂SO₄, filtered and concentrated. The residue was purified by column chromatography over silicagel to give tert-butyl (2R)-2-(tert-butyldimethyl-silyloxy)-3-(4-chloro-3-(4-(4-iodo-1-methyl-1H-pyrazol-5-yl)-5-methyl-6-(5H-pyrrolo[3,4-b]pyridin-6(7H)-yl)pyrimidin-2-yl)phenoxy)propyl(methyl)carbamate (160 mg, 95% yield). ESI-LCMS (m/z): 846 found for [M+H]+.

### Step 3: (2R)-1-(4-chloro-3-(4-(4-iodo-1-methyl-1H-pyrazol-5-yl)-5-methyl-6-(5H-pyrrolo[3,4-b]pyridin-6(7H)-yl)pyrimidin-2-yl)phenoxy)-3-(methyl-amino)propan-2-ol

A solution of tert-butyl (2R)-2-(tert-butyldimethylsilyloxy)-3-(4-chloro-3-(4-(4-iodo-1-methyl-1H-pyrazol-5-yl)-5-methyl-6-(5H-pyrrolo[3,4-b]pyridin-6(7H)-yl)pyrimidin-2-yl) phenoxy)propyl(methyl)carbamate (160 mg, 0.19 mmol) was treated with 90% TFA (2.2 mL), and the mixture was stirred at room temperature for 16 h. The mixture was concentrated in vacuo and the residue was dissolved in MeOH (2 ml). The solution was adjusted to pH 7-8 with aqueous K₂CO₃ solution, then the mixture was filtered. The filtrate was concentrated, and the residue was purified by preparative HPLC to give (2R)-1-(4-chloro-3-(4-(4-iodo-1-methyl-1H-pyrazol-5-yl)-5-methyl-6-(5H-pyrrolo[3,4-b]pyridin-6(7H)-yl)pyrimidin-2-yl)phenoxy)-3-(methylamino) propan-2-ol (91 mg, 76% yield). ESI-LCMS (m/z): 632.1 found for [M+H]+; ¹HNMR (400 MHz, CD₃OD) δ ppm: 8.50 (d, J=4.8 Hz, 1H), 7.89 (d, J=7.2 Hz, 1H), 7.65 (s, 1H), 7.43-7.38 (m, 2H), 7.37 (d, J=3.2 Hz, 1H), 7.05 (dd, J=3.2 and 8.8 Hz, 1H), 5.45-5.37 (m, 2H), 5.30-5.21 (m, 2H), 4.12-4.09 (m, 1H), 4.06-3.98 (m, 2H), 3.90 (s, 3H), 2.84-2.71 (m, 2H), 2.47 (s, 3H), 2.46 (s, 3H).

### Example 5. Preparation of (2R)-1-(4-chloro-3-(4-(4-chloro-1-ethyl-1H-pyrazol-5-yl)-5-methyl-6-(5H-pyrrolo[3,4-b]pyridin-6(7H)-yl)pyrimidin-2-yl)phenoxy)-3-(methyl-amino)propan-2-ol

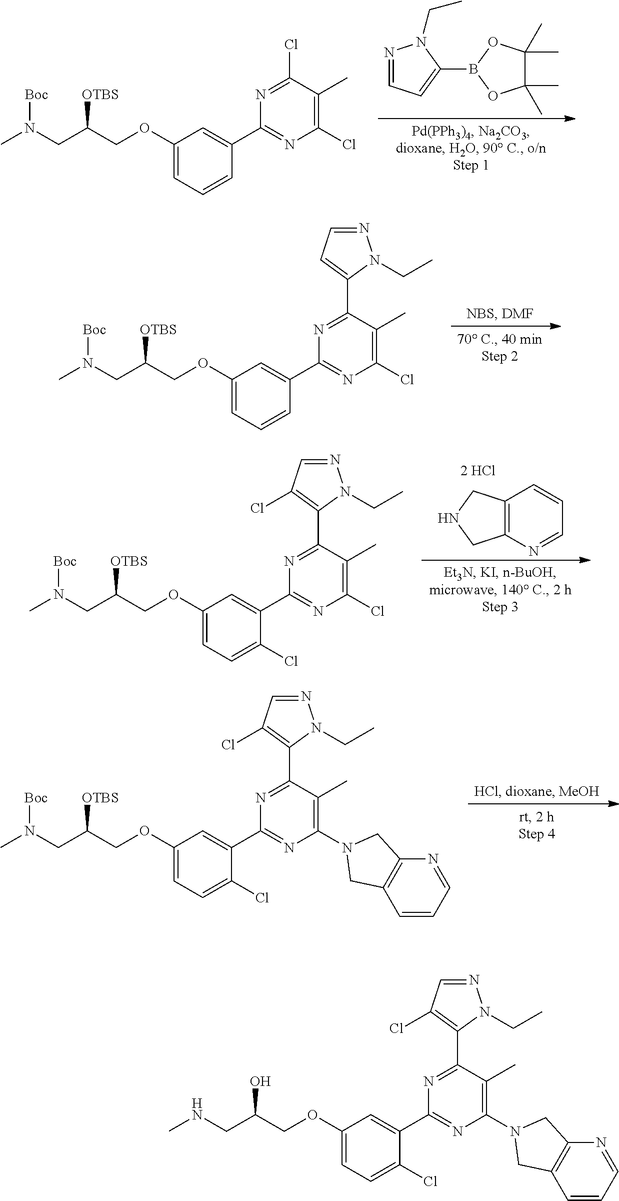

### Step 1: tert-butyl (R)-2-(tert-butyldimethylsilyloxy)-3-(3-(4-chloro-6-(1-ethyl-1H-pyrazol-5-yl)-5-methylpyrimidin-2-yl)phenoxy)propyl(methyl) carbamate

To a solution of (R)-tert-butyl 2-(tert-butyldimethylsilyloxy)-3-(3-(4,6-dichloro-5-methyl-pyrimidin-2-yl)phenoxy)propyl(methyl)carbamate (1.0 g, 1.7 mmol) in degassed dioxane/H₂O (5/1, 30 mL) was added 1-ethyl-5-(4,4,5,5-tetra-methyl-1,2-oxaborolan-2-yl)-1H-pyrazole (420 mg, 1.9 mmol); Pd(PPh₃)₄(104 mg, 0.09 mmol) and Na₂CO₃ (572 mg, 5.4 mmol) at room temperature. The system was purged with N₂ and the mixture was stirred at 90° C. for 16 h. After being cooled down to room temperature, the solvent was removed in vacuo. The residue was diluted with water (30 mL) and extracted with EtOAc (100 mL×2). The combined organic layers were washed with water (30 mL) and brine (30 mL), dried over Na₂SO₄, filtered and concentrated. The residue was purified by column chromatography over silicagel (petroleum ether/EtOAc=2/1) to give tert-butyl (R)-2-(tert-butyldimethyl-silyloxy)-3-(3-(4-chloro-6-(1-ethyl-1H-pyrazol-5-yl)-5-methylpyrimidin-2-yl) phenoxy)propyl(methyl)carbamate (540 mg, white solid, 49% yield). ESI-LCMS (m/z): 638.4 found for [M+23]⁺.

### Step 2: tert-butyl (R)-2-(tert-butyldimethylsilyloxy)-3-(4-chloro-3-(4-chloro-6-(4-chloro-1-ethyl-1H-pyrazol-5-yl)-5-methylpyrimidin-2-yl)phenoxy) propyl(methyl) carbamate

To a solution of tert-butyl (R)-2-(tert-butyldimethylsilyloxy)-3-(3-(4-chloro-6-(1-ethyl-1H-pyrazol-5-yl)-5-methylpyrimidin-2-yl)phenoxy)propyl(methyl)carbamate (400 mg, 0.65 mmol) in 30 mL of DMF was added NCS (389 mg, 2.92 mmol) and the mixture was heated at 70° C. for 40 min; cooled down to room temperature, diluted with EtOAc (30 mL) and washed with water (30 mL×2) and brine (30 mL). The organic layer was dried over Na₂SO₄, filtered and concentrated and the resulting residue was purified by preparative TLC (petroleum ether/EtOAc=3/1) to give tert-butyl (R)-2-(tert-butyldimethylsilyloxy)-3-(4-chloro-3-(4-chloro-6-(4-chloro-1-ethyl-1H-pyrazol-5-yl)-5-methylpyrimidin-2-yl)phenoxy)propyl(methyl) carbamate (390 mg, white solid, 88% yield). ESI-LCMS (m/z): 630.4 found for [M−56]⁺.

### Step 3: tert-butyl (R)-2-(tert-butyldimethylsilyloxy)-3-(4-chloro-3-(4-(4-chloro-1-ethyl-1H-pyrazol-5-yl)-5-methyl-6-(5H-pyrrolo[3,4-b]pyridin-6 (7H)-yl)pyrimidin-2-yl)phenoxy)propyl(methyl)carbamate

A reaction pressure vessel was charged with a mixture of tert-butyl (R)-2-(tert-butyldimethylsilyloxy)-3-(4-chloro-3-(4-chloro-6-(4-chloro-1-ethyl-1H-pyrazol-5-yl)-5-methylpyrimidin-2-yl)phenoxy)propyl (methyl) carbamate (100 mg, 0.15 mmol); 6,7-dihydro-5H-pyrrolo[3,4-b]pyridine dihydrochloride (or any other suitably substituted primary or secondary amine, 0.35 mmol), KI (30 mg, 0.18 mmol), triethylamine (2 mL) and n-BuOH (4 mL). The vessel was capped, placed in a microwave reactor and irradiated for 2 h. at external temperature of 140° C. After being cooled down to room temperature, 30 mL of water was added and the mixture was extracted with EtOAc (40 mL×3). The combined organic layers were washed with water (30 mL) and brine (30 mL), dried over Na₂SO₄, filtered and concentrated to give tert-butyl (R)-2-(tert-butyldimethylsilyloxy)-3-(4-chloro-3-(4-(4-chloro-1-ethyl-1H-pyrazol-5-yl)-5-methyl-6-(5H-pyrrolo[3,4-b]pyridin-6(7H)-yl)pyrimidin-2-yl)phenoxy)propyl (methyl) carbamate (128 mg, crude), which was used for next step directly without further purification. ESI-LCMS (m/z): 768.4 found for [M+1]⁺.

### Step 4: (2R)-1-(4-chloro-3-(4-(4-chloro-1-ethyl-1H-pyrazol-5-yl)-5-methyl-6-(5H-pyrrolo[3,4-b]pyridin-6(7H)-yl)pyrimidin-2-yl)phenoxy)-3-(methyl-amino)propan-2-ol

A solution of tert-butyl (R)-2-(tert-butyldimethylsilyloxy)-3-(4-chloro-3-(4-(4-chloro-1-ethyl-1H-pyrazol-5-yl)-5-methyl-6-(5H-pyrrolo[3,4-b]pyridin-6(7H)-yl)pyrimidin-2-yl)phenoxy) propyl(methyl)carbamate (128 mg, crude from step 3) in MeOH (2 mL) was treated with 2 mL of 4N HCl solution in dioxane and the mixture was stirred at room temperature for 2 h. The solvent was then removed in vacuo, the resulting residue was dissolved in MeOH (5 mL) and treated with ammonia till pH 8-9. The mixture was concentrated under vacuum and the residue was purified by preparative HPLC to give (2R)-1-(4-chloro-3-(4-(4-chloro-1-ethyl-1H-pyrazol-5-yl)-5-methyl-6-(5H-pyrrolo[3,4-b]pyridine-6(7H)-yl) pyrimidin-2-yl)phenoxy)-3-(methylamino)propan-2-ol as white solid (60 mg, 72% yield for two steps). ESI-LCMS: 554.0 found for [M+1]+; ¹HNMR (400 MHz, CD₃OD) δ ppm: 8.49 (d, J=4.8 Hz, 1H), 7.87 (d, J=7.2 Hz, 1H), 7.62 (s, 1H), 7.44-7.37 (m, 2H), 7.34 (d, J=3.2 Hz, 1H), 7.05 (dd, J=2.8 and 8.8 Hz, 1H), 5.43 (t, J=15.2 Hz, 2H), 5.20 (t, J=17.6 Hz, 2H), 4.35-4.26 (m, 1H), 4.21-4.10 (m, 2H), 4.06-3.98 (m, 2H), 2.88-2.74 (m, 2H), 2.49 (s, 3H), 2.48 (s, 3H), 1.36 (t, J=7.2 Hz, 3H).

### Example 6. Preparation of (2R)-1-(4-chloro-3-(4-(3,5-dimethylisoxazol-4-yl)-5-methyl-6-(6-(2,2,2-trifluoroethyl)-2,6-diazaspiro[3.3]heptan-2-yl)pyrimidin-2-yl)phenoxy)-3-(methylamino)propan-2-ol

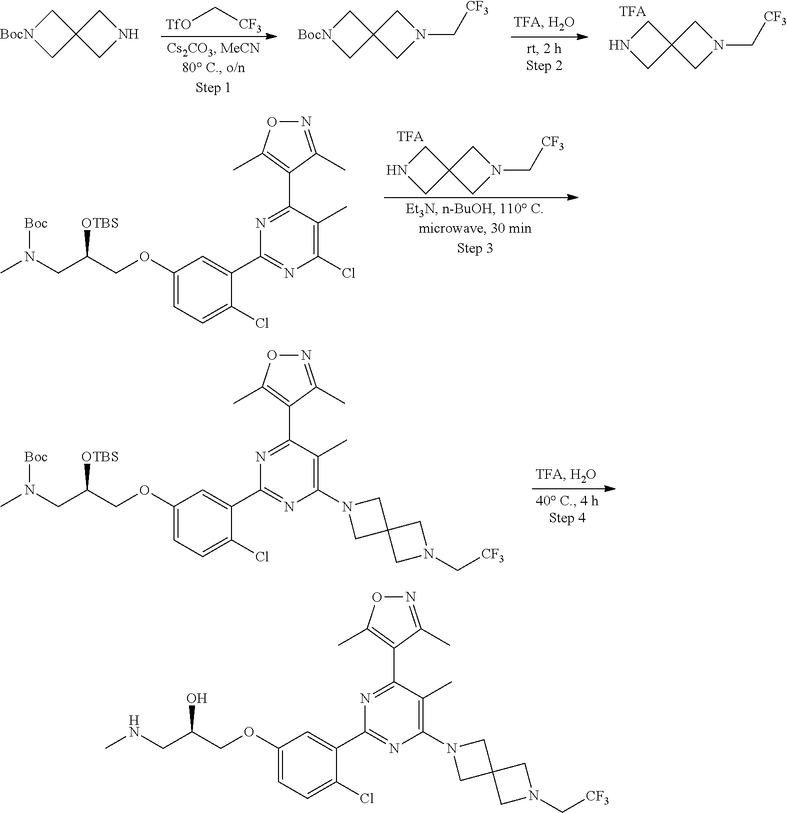

### Step 1: tert-butyl 6-(2,2,2-trifluoroethyl)-2,6-diazaspiro[3.3]heptane-2-carboxylate

To a solution of tert-butyl 2,6-diazaspiro[3.3]heptane-2-carboxylate (200 mg, 1.01 mmol) in MeCN (6 mL) was added 3,3,3-trifluoropropyl trifluoromethanesulfonate (403 mg, 1.65 mmol) and Cs₂CO₃ (804 mg, 2.47 mmol) at room temperature. The reaction mixture was stirred at 80° C. overnight, cooled down to room temperature, diluted with water (80 mL) and extracted with EtOAc (60 mL×3). The combined organic layers were washed with water (30 mL) and brine (30 mL), dried over Na₂SO₄, filtered and concentrated in vacuo to give tert-butyl 6-(2,2,2-trifluoroethyl)-2,6-diazaspiro[3.3]heptane-2-carboxylate as white solid (250 mg, crude), which was used into next step directly without further purification. ESI-LCMS (m/z): 281.1 found for [M+1]+.

### Step 2: 2-(2,2,2-trifluoroethyl)-2,6-diazaspiro[3.3]heptane TFA salt

A solution of tert-butyl 6-(2,2,2-trifluoroethyl)-2,6-diazaspiro[3.3]heptane-2-carboxylate (250 mg, crude from step 1) in TFA (5 mL) and water (0.5 ml) was stirred at room temperature for 2 h. The solvent was then removed in vacuo to give 2-(2,2,2-trifluoroethyl)-2,6-diaza-spiro[3.3]heptane 2,2,2-trifluoroacetate trifluoroacetate salt as brown solid (920 mg, crude), which was used into next step directly without further purification. ¹HNMR (400 MHz, CDCl₃) δ ppm: 4.36 (s, 4H), 4.29 (s, 4H), 3.97-3.90 (m, 2H).

### Step 3: tert-butyl (2R)-2-(tert-butyldimethylsilyloxy)-3-(4-chloro-3-(4-(3,5-dimethylisoxazol-4-yl)-5-methyl-6-(6-(2,2,2-trifluoroethyl)-2,6-diazaspiro[3.3]heptan-2-yl) pyrimidin-2-yl)phenoxy)propyl(methyl)carbamate

A reaction pressure vessel was charged with a mixture of tert-butyl (R)-2-(tert-butyldimethylsilyloxy)-3-(4-chloro-3-(4-chloro-6-(3,5-dimethylisoxazol-4-yl)-5-methyl pyrimidin-2-yl)phenoxy)propyl(methyl) carbamate (100 mg, 0.15 mmol); 2-(2,2,2-trifluoroethyl)-2,6-diazaspiro[3.3]heptane TFA salt (or any other suitably substituted primary or secondary amine, 1.54 mmol), triethylamine (186 mg, 1.85 mmol) and n-BuOH (1 mL). The vessel was capped, placed in a microwave reactor and irradiated for 30 min. at external temperature of 110° C.; cooled down to room temperature, diluted with water (70 mL) and extracted with EtOAc (60 mL×3). The organic layers were combined, concentrated in vacuo and the residue was purified by preparative TLC developed with petroleum ether/EtOAc=2/1 to give tert-butyl (2R)-2-(tert-butyldimethyl-silyloxy)-3-(4-chloro-3-(4-(3,5-dimethylisoxazol-4-yl)-5-methyl-6-(6-(2,2,2-trifluoro-ethyl)-2,6-diazaspiro[3.3]heptan-2-yl)pyrimidin-2-yl)phenoxy)propyl(methyl) carbamate as light yellow solid (110 mg, 89.7%). ESI-LCMS (m/z): 795.3 found for [M+1]⁺.

### Step 4: (2R)-1-(4-chloro-3-(4-(3,5-dimethylisoxazol-4-yl)-5-methyl-6-(6-(2,2,2-trifluoroethyl)-2,6-diazaspiro[3.3]heptan-2-yl)pyrimidin-2-yl)phenoxy)-3-(methylamino)propan-2-ol

A solution of tert-butyl (2R)-2-(tert-butyldimethylsilyloxy)-3-(4-chloro-3-(4-(3,5-dimethyl-isoxazol-4-yl)-5-methyl-6-(6-(2,2,2-trifluoroethyl)-2,6-diazaspiro[3.3]heptan-2-yl) pyrimidin-2-yl) phenoxy)propyl(methyl)carbamate (110 mg, 0.14 mmol) in TFA (5 mL) and water (0.5 ml), was stirred at 40° C. for 8 h. The solvent was then removed in vacuo, the residue was dissolved in MeOH (3 ml) and the solution was adjusted to pH 9 with ammonia. The solvent was removed in rotary evaporator and the residue was purified by preparative HPLC to give (2R)-1-(4-chloro-3-(4-(3,5-dimethylisoxazol-4-yl)-5-methyl-6-(6-(2,2,2-trifluoroethyl)-2,6-diazaspiro[3.3]heptan-2-yl)pyrimidin-2-yl)phenoxy)-3-(methylamino) propan-2-ol as a white solid (26 mg, 32%). ESI-LCMS (m/z): 581.1 found for [M+H]+; ¹HNMR (400 MHz, CD₃OD) δ ppm: 7.39 (d, J=8.8 Hz, 1H), 7.19 (d, J=2.8 Hz, 1H), 7.05 (dd, J=3.2 and 8.8 Hz, 1H), 4.50 (s, 4H), 4.14-4.07 (m, 1H), 4.05-3.96 (m, 2H), 3.65 (s, 4H), 3.20-3.12 (m, 2H), 2.85-2.70 (m, 2H), 2.46 (s, 3H), 2.37 (s, 3H), 2.24 (s, 3H), 2.14 (s, 3H).

### Example 7. Preparation of (2R)-1-(4-chloro-3-(4-(3-chloro-1,4-dimethyl-1H-pyrazol-5-yl)-5-methyl-6-(6-(2,2,2-trifluoroethyl)-2,6-diazaspiro[3.3]heptan-2-yl)pyrimidin-2-yl)phenoxy)-3-(methylamino)propan-2-ol

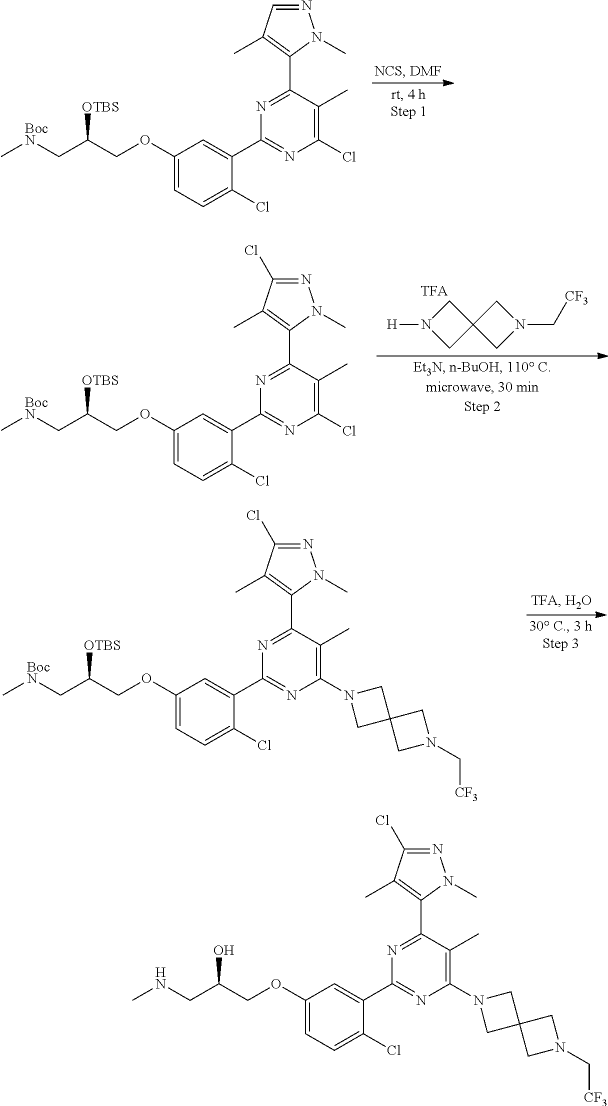

### Step 1: tert-butyl (2R)-2-(tert-butyldimethylsilyloxy)-3-(4-chloro-3-(4-chloro-6-(3-chloro-1,4-dimethyl-1H-pyrazol-5-yl)-5-methylpyrimidin-2-yl) phenoxy)propyl(methyl)carbamate

A solution of tert-butyl (2R)-2-(tert-butyldimethylsilyloxy)-3-(4-chloro-3-(4-chloro-6-(1,4-dimethyl-1H-pyrazol-5-yl)-5-methylpyrimidin-2-yl)phenoxy)propyl(methyl) carbamate (1.66 g, 2.55 mmol) and N-chlorosuccinimide (509 mg, 3.83 mmol) in DMF (20 mL) was stirred at room temperature for 12 h. After the reaction was complete, water (50 mL) and ethyl acetate (50 mL) were added. The organic layer was separated and washed with water (50 mL×4) and brine (50 mL), dried over Na₂SO₄, filtered and concentrated. The residue was purified by column chromatography over silicagel eluted with petroleum ether/ethyl acetate=5:1 to give tert-butyl (2R)-2-(tert-butyldimethylsilyloxy)-3-(4-chloro-3-(4-chloro-6-(3-chloro-1,4-dimethyl-1H-pyrazol-5-yl)-5-methylpyrimidin-2-yl)phenoxy)propyl(methyl)carbamate (420 mg, 24% yield) as a colorless oil. ESI-LCMS (m/z): 706.2 found for [M+Na]⁺.

### Step 2: tert-butyl (2R)-2-(tert-butyldimethylsilyloxy)-3-(4-chloro-3-(4-(3-chloro-1,4-dimethyl-1H-pyrazol-5-yl)-5-methyl-6-(6-(2,2,2-trifluoroethyl)-2,6-diazaspiro[3.3]heptan-2-yl)pyrimidin-2-yl)phenoxy)propyl(methyl)carbamate

A reaction pressure vessel was charged with a mixture of tert-butyl (2R)-2-(tert-butyldimethylsilyloxy)-3-(4-chloro-3-(4-chloro-6-(3-chloro-1,4-dimethyl-1H-pyrazol-5-yl)-5-methylpyrimidin-2-yl)phenoxy)propyl(methyl)carbamate (200 mg, 0.29 mmol); 2-(2,2,2-trifluoroethyl)-2,6-diazaspiro[3.3]heptane TFA salt (or any other suitably substituted primary or secondary amine, 0.35 mmol), DIPEA (151 mg, 1.17 mmol) and DMSO (4 mL). The vessel was capped, placed in a microwave reactor and irradiated for 30 min. at external temperature of 140° C. After being cooled down to room temperature, the mixture was diluted with water (50 mL) and extracted with EtOAc (50 mL×2). The combined organic layers were washed with water (50 mL×4) and brine (40 mL), dried over Na₂SO₄, filtered and concentrated to give tert-butyl (2R)-2-(tert-butyl dimethyl silyloxy)-3-(4-chloro-3-(4-(3-chloro-1,4-dimethyl-1H-pyrazol-5-yl)-5-methyl-6-(6-(2,2,2-trifluoro-ethyl)-2,6-diazaspiro[3.3]heptan-2-yl)pyrimidin-2-yl)phenoxy)propyl (methyl) carbamate (270 mg, crude) as a brown oil, which was used directly in the next step without further purification. ESI-LCMS (m/z): 828.0 found for [M+H]⁺.

### Step 3: (2R)-1-(4-chloro-3-(4-(3-chloro-1,4-dimethyl-1H-pyrazol-5-yl)-5-methyl-6-(6-(2,2,2-trifluoroethyl)-2,6-diazaspiro 3.3]heptan-2-yl)pyrimidin-2-yl)phenoxy)-3-(methylamino)propan-2-ol

A solution of tert-butyl(2R)-2-(tert-butyldimethylsilyloxy)-3-(4-chloro-3-(4-(3-chloro-1,4-dimethyl-1H-pyrazol-5-yl)-5-methyl-6-(6-(2,2,2-trifluoroethyl)-2,6-diazaspiro[3.3]heptan-2-yl)pyrimidin-2-yl)phenoxy)propyl(methyl)carbamate (270 mg, crude, from step 2) in 90% TFA (5 mL) was stirred at 30° C. for 3 h. The solvent was then removed in vacuo, the residue was dissolved in MeOH (5 ml) and the solution was adjusted to pH 7-8 with ammonia. The solvent was removed in rotary evaporator and the residue was purified by preparative HPLC to give (2R)-1-(4-chloro-3-(4-(3-chloro-1,4-dimethyl-1H-pyrazol-5-yl)-5-methyl-6-(6-(2,2,2-trifluoroethyl)-2,6-diazaspiro[3.3]heptan-2-yl)pyrimidin-2-yl)phenoxy)-3-(methylamino)propan-2-ol as a white solid (27 mg, 15% yield for 2 steps). ESI-LCMS (m/z): 614.0 found for [M+H]+; ¹HNMR (400 MHz, MeOD) δ ppm: 7.39 (d, J=8.8 Hz, 1H), 7.21 (d, J=3.2 Hz, 1H), 7.04 (dd, J=3.2 and 8.8 Hz, 1H), 4.58-4.49 (m, 4H), 4.13-4.07 (m, 1H), 4.05-3.96 (m, 2H), 3.72 (s, 3H), 3.65 (s, 4H), 3.16 (q, J=9.6 Hz, 2H), 2.86-2.72 (m, 2H), 2.48 (s, 3H), 2.13 (s, 3H), 1.95 (s, 3H).

### Example 8. Preparation of (2R)-1-(3-(4-(3-bromo-1,4-dimethyl-1H-pyrazol-5-yl)-5-methyl-6-(6-(2,2,2-trifluoroethyl)-2,6-diazaspiro[3.3]heptan-2-yl)pyrimidin-2-yl)-4-chloro-phenoxy)-3-(methylamino)propan-2-ol

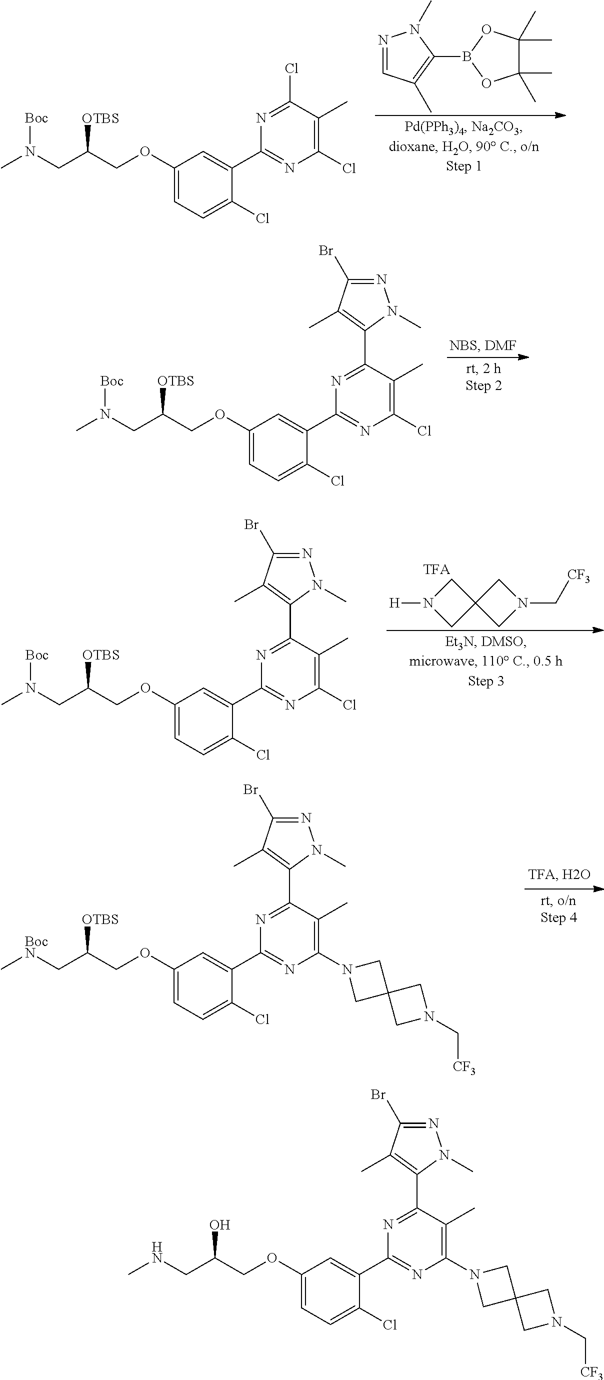

### Step 1: tert-butyl (2R)-2-(tert-butyldimethylsilyloxy)-3-(4-chloro-3-(4-chloro-6-(1,4-dimethyl-1H-pyrazol-5-yl)-5-methylpyrimidin-2-yl)phenoxy) propyl(methyl)carbamate

To a solution of (R)-tert-butyl 2-(tert-butyldimethylsilyloxy)-3-(4-chloro-3-(4,6-dichloro-5-methylpyrimidin-2-yl)phenoxy)propyl(methyl)carbamate (1.0 g, 1.7 mmol) in degassed dioxane and H₂O (3/1, 20 mL) was added Na₂CO₃ (541 mg, 5.1 mmol), Pd(PPh₃)₄(98 mg, 0.08 mmol) and 1,4-dimethyl-5-(4,4,5,5-tetramethyl-1,3,2-dioxa-borolan-2-yl)-1H-pyrazole (755 mg, 3.4 mmol). The system was purged with N₂ and the mixture was stirred at 90° C. for 16 h. After being cooled down to room temperature, the solvent was removed in vacuo. The residue was diluted with water (30 mL) and extracted with EtOAc (100 mL×2). The combined organic layers were washed with water (30 mL) and brine (30 mL), dried over Na₂SO₄, filtered and concentrated. The residue was purified by column chromatography over silicagel (petroleum ether/EtOAc=4/1) to give tert-butyl (2R)-2-(tert-butyldimethylsilyloxy)-3-(4-chloro-3-(4-chloro-6-(1,4-dimethyl-1H-pyrazol-5-yl)-5-methylpyrimidin-2-yl)phenoxy)propyl(methyl) carbamate (520 mg, 47% yield) as white solid. ESI-LCMS (m/z): 650 found for [M+1]⁺.

### Step 2: tert-butyl (2R)-3-(3-(4-(3-bromo-1,4-dimethyl-1H-pyrazol-5-yl)-6-chloro-5-methylpyrimidin-2-yl)-4-chlorophenoxy)-2-(tert-butyldimethyl-silyloxy)propyl(methyl)carbamate

A solution of (2R)-2-(tert-butyldimethylsilyloxy)-3-(4-chloro-3-(4-chloro-6-(1,4-dimethyl-1H-pyrazol-5-yl)-5-methylpyrimidin-2-yl)phenoxy)propyl(methyl) carbamate (520 mg, 0.8 mmol) and NBS (470 mg, 2.64 mmol) in DMF (5 ml) was stirred at room temperature for 2 h. The mixture was diluted with water (50 mL) and extracted with EtOAc (20 mL×3). The combined organic layers were washed with water (20 mL) and brine (20 mL), dried over Na₂SO₄, filtered and concentrated to give tert-butyl (2R)-3-(3-(4-(3-bromo-1,4-dimethyl-1H-pyrazol-5-yl)-6-chloro-5-methylpyrimidin-2-yl)-4-chlorophenoxy)-2-(tert-butyldimethylsilyloxy)propyl(methyl)carbamate (600 mg, 103% yield). ESI-LCMS (m/z): 750 found for [M+23]⁺.

### Step 3: tert-butyl (2R)-3-(3-(4-(3-bromo-1,4-dimethyl-1H-pyrazol-5-yl)-5-methyl-6-(6-(2,2,2-trifluoroethyl)-2,6-diazaspiro[3.3]heptan-2-yl)pyrimidin-2-yl)-4-chlorophenoxy)-2-(tert-butyldimethylsilyloxy)propyl(methyl)carbamate

A reaction pressure vessel was charged with a mixture of tert-butyl (2R)-3-(3-(4-(3-bromo-1,4-dimethyl-1H-pyrazol-5-yl)-6-chloro-5-methylpyrimidin-2-yl)-4-chlorophenoxy)-2-(tert-butyldimethylsilyloxy) propyl(methyl)carbamate (130 mg, 0.18 mmol); 2-(2,2,2-trifluoroethyl)-2,6-diazaspiro[3.3]heptane TFA salt (or any other suitably substituted primary or secondary amine, 0.36 mmol), triethylamine (0.5 mL, 3.5 mmol). and DMSO (3 mL). The vessel was capped, placed in a microwave reactor and irradiated for 30 min. at external temperature of 110° C. After being cooled down to room temperature, the mixture was diluted with water (15 mL) and extracted with EtOAc (20 mL×3). The combined organic layers were washed with water (20 mL) and brine (20 mL), dried over Na₂SO₄, filtered and concentrated and the residue was submitted to purification by column chromatography over silicagel to give tert-butyl (2R)-3-(3-(4-(3-bromo-1,4-dimethyl-1H-pyrazol-5-yl)-5-methyl-6-(6-(2,2,2-trifluoroethyl)-2,6-diazaspiro[3.3]heptan-2-yl)pyrimidin-2-yl)-4-chlorophenoxy)-2-(tert-butyldimethylsilyloxy) propyl (methyl) carbamate (120 mg, 76% yield) as white solid. ESI-LCMS (m/z): 872.2 found for [M+H]⁺.

### Step 4: (2R)-1-(3-(4-(3-bromo-1,4-dimethyl-1H-pyrazol-5-yl)-5-methyl-6-(6-(2,2,2-trifluoroethyl)-2,6-diazaspiro[3.3]heptan-2-yl)pyrimidin-2-yl)-4-chlorophenoxy)-3-(methylamino)propan-2-ol

A solution of tert-butyl (2R)-3-(3-(4-(3-bromo-1,4-dimethyl-1H-pyrazol-5-yl)-5-methyl-6-(6-(2,2,2-trifluoroethyl)-2,6-diazaspiro[3.3]heptan-2-yl)pyrimidin-2-yl)-4-chlorophenoxy)-2-(tert-butyldimethylsilyloxy)propyl(methyl)carbamate (100 mg, 0.11 mmol) in 90% TFA (2 mL) was stirred at room temperature for 16 h. After removal of volatiles in vacuo, the residue was dissolved in MeOH (2 ml), the solution pH was adjusted to 7-8 with aqueous K₂CO₃ solution; the mixture was filtered and the filtrate was concentrated. The resulting residue was purified by preparative HPLC to give (2R)-1-(3-(4-(3-bromo-1,4-dimethyl-1H-pyrazol-5-yl)-5-methyl-6-(6-(2,2,2-trifluoroethyl)-2,6-diazaspiro[3.3]heptan-2-yl)pyrimidin-2-yl)-4-chlorophenoxy)-3-(methyl amino)propan-2-ol (23 mg, 32% yield) as white solid. ESI-LCMS (m/z): 658.1 found for [M+H]+; ¹HNMR (400 MHz, CD₃OD) δ ppm:: 7.39 (d, J=8.8 Hz, 1H), 7.21 (d, J=2.8 Hz, 1H), 7.04 (dd, J=2.8 and 8.8 Hz, 1H), 4.60-4.50 (m, 4H), 4.13-4.08 (m, 1H), 4.04-3.98 (m, 2H), 3.75 (s, 3H), 3.65 (s, 4H), 3.21-3.12 (m, 2H), 2.88-2.75 (m, 2H), 2.49 (s, 3H), 2.12 (s, 3H), 1.94 (s, 3H).

### Example 9. Preparation of methyl (R)-5-(2-(2-chloro-5-(2-hydroxy-3-(methylamino)propoxy)phenyl)-6-(3,5-dimethylisoxazol-4-yl)-5-methylpyrimidin-4-yl)-3,4,5,6-tetrahydropyrrolo[3,4-c]pyrrole-2(1H)-carboxylate

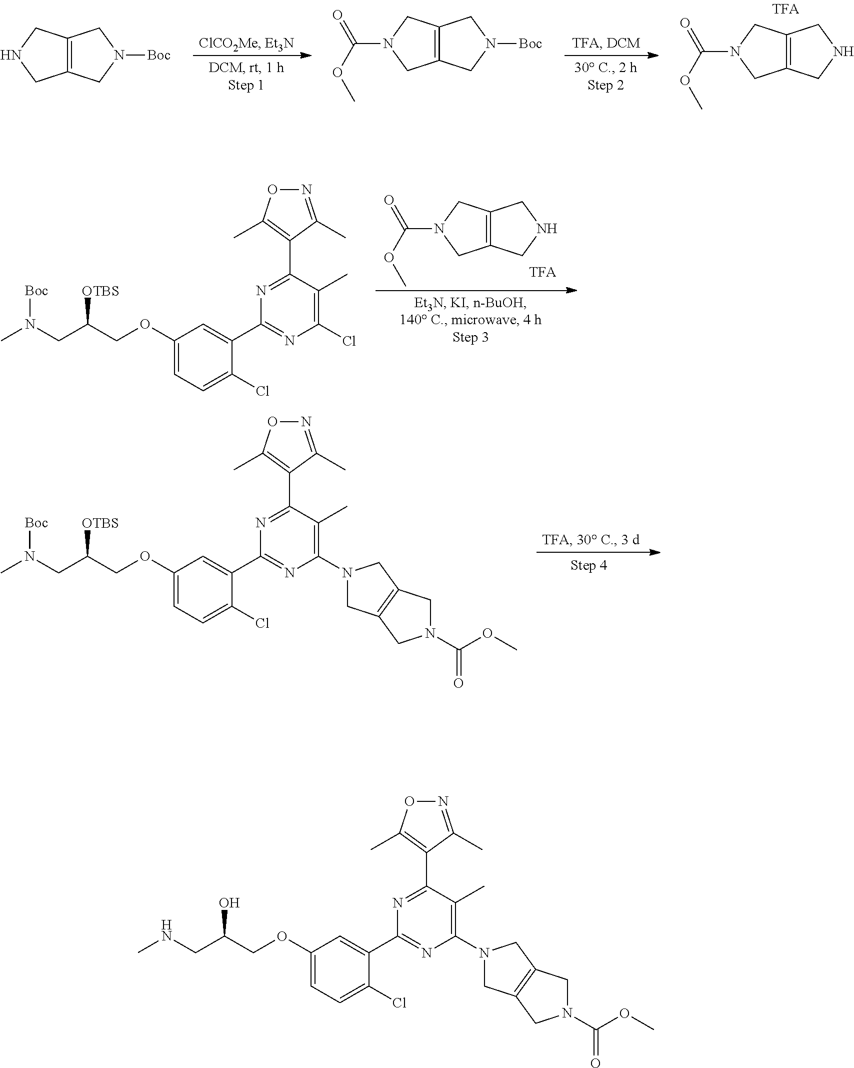

### Step 1: 2-(tert-butyl) 5-methyl 4,6-dihydropyrrolo[3,4-c]pyrrole-2,5(1H,3H)-dicarboxylate

To a solution tert-butyl 3,4,5,6-tetrahydropyrrolo[3,4-c]pyrrole-2(1H)-carboxylate (100 mg, 0.404 mmol) and triethylamine (82 mg, 0.80 mmol) in 5 mL of DCM was added and ClCO₂Me (58 mg, 0.61 mmol) and the mixture was stirred at room temperature for 1 h. After the reaction was complete, water (10 mL) was added and the mixture was extracted with DCM (15 mL×3). The combined organic layers were washed with brine (20 mL), dried over Na₂SO₄, filtered and concentrated to give 2-(tert-butyl) 5-methyl 4,6-dihydropyrrolo[3,4-c]pyrrole-2,5(1H,3H)-dicarboxylate (98 mg, 90% yield). ESI-LCMS (m/z): 269.7 found for [M+1]⁺.

### Step 2: methyl 3,4,5,6-tetrahydropyrrolo[3,4-c]pyrrole-2(1H)-carboxylate TFA salt

A solution of 2-(tert-butyl) 5-methyl 4,6-dihydropyrrolo[3,4-c]pyrrole-2,5(1H,3H)-dicarboxylate (98 mg, 0.37 mmol) in DCM (3 mL) was treated with TFA (2 mL) and the mixture was stirred at 30° C. for 2 h. The solvent was removed in vacuo to give methyl 3,4,5,6-tetrahydropyrrolo[3,4-c]pyrrole-2(1H)-carboxylate TFA salt (210 mg, 100% yield), which was used directly for the next step without further purification. ESI-LCMS (m/z): 169.7 found for [M+1]⁺.

### Step 3: methyl (R)-5-(2-(5-(3-((tert-butoxycarbonyl)(methyl)amino)-2-((tert-butyldimethylsilyl)oxy)propoxy)-2-chlorophenyl)-6-(3,5-dimethylisoxazol-4-yl)-5-methyl pyrimidin-4-yl)-3,4,5,6-tetrahydropyrrolo[3,4-c]pyrrole-2(1H)-carboxylate

A reaction pressure vessel was charged with a mixture of tert-butyl (R)-2-(tert-butyldimethylsilyloxy)-3-(4-chloro-3-(4-chloro-6-(3,5-dimethylisoxazol-4-yl)-5-methyl pyrimidin-2-yl)phenoxy)propyl(methyl) carbamate (150 mg, 0.23 mmol); methyl 3,4,5,6-tetrahydropyrrolo[3,4-c]pyrrole-2(1H)-carboxylate TFA salt, (or any other suitably substituted primary or secondary amine, 0.79 mmol), KI (30 mg, 0.18 mmol), triethylamine (2 mL). and n-BuOH (3 mL). The vessel was capped, placed in a microwave reactor and irradiated for 2 h. at external temperature of 140° C. After being cooled down to room temperature, 20 mL of water was added and the mixture was extracted with EtOAc (20 mL×3). The combined organic layers were washed with water (30 mL) and brine (30 mL), dried over Na₂SO₄, filtered and concentrated. The residue was purified by preparative TLC (MeOH/CH₂Cl₂=1/25) to give methyl (R)-5-(2-(5-(3-((tert-butoxycarbonyl)(methyl)amino)-2-((tert-butyldimethylsilyl)oxy)propoxy)-2-chloro phenyl)-6-(3,5-dimethylisoxazol-4-yl)-5-methylpyrimidin-4-yl)-3,4,5,6-tetrahydropyrrolo[3,4-c]pyrrole-2(1H)-carboxylate (145 mg, 81% yield). ESI-LCMS (m/z): 783.4 found for [M+1]⁺.

### Step 4: methyl (R)-5-(2-(2-chloro-5-(2-hydroxy-3-(methylamino)propoxy)phenyl)-6-(3,5-dimethylisoxazol-4-yl)-5-methylpyrimidin-4-yl)-3,4,5,6-tetrahydropyrrolo[3,4-c]pyrrole-2(1H)-carboxylate

A solution of methyl (R)-5-(2-(5-(3-((tert-butoxycarbonyl)(methyl)amino)-2-((tert-butyldimethylsilyl)oxy) propoxy)-2-chlorophenyl)-6-(3,5-dimethylisoxazol-4-yl)-5-methyl pyrimidin-4-yl)-3,4,5,6-tetrahydropyrrolo[3,4-c]pyrrole-2(1H)-carboxylate (100 mg, 0.12 mmol) in 90% TFA (3 ml) was stirred at 30° C. for 72 h. The solvent was then removed in vacuo and the residue was dissolved in MeOH (5 mL). Ammonia was added to adjust the pH to 8-9; the mixture was concentrated under vacuum and the residue was purified by preparative HPLC to give methyl (R)-5-(2-(2-chloro-5-(2-hydroxy-3-(methylamino) propoxy)phenyl)-6-(3,5-dimethylisoxazol-4-yl)-5-methylpyrimidin-4-yl)-3,4,5,6-tetrahydro pyrrolo[3,4-c]pyrrole-2(1H)-carboxylate (45 mg, 62% yield). ESI-LCMS: 569.1 found for [M+1]; 1HNMR (400 MHz, CD₃OD) δ ppm: 7.39 (d, J=8.8 Hz, 1H), 7.27 (d, J=2.8 Hz, 1H) 7.06-7.00 (m, 1H), 4.71 (br s, 4H), 4.25 (br s, 4H), 4.13-4.08 (m, 1H), 4.06-3.98 (m, 2H), 3.76 (s, 3H), 2.84-2.70 (m, 2H), 2.47 (s, 3H), 2.41 (s, 3H), 2.36 (s, 3H), 2.27 (s, 3H).

### Example 10. Preparation of (R)-1-(4-chloro-3-(4-(3,5-dimethylisoxazol-4-yl)-5-methyl-6-(5-(2,2,2-trifluoroethyl)-1,3,4,5,6,7-hexahydro-2H-pyrrolo[3,4-c]pyridin-2-yl)pyrimidin-2-yl) phenoxy)-3-(methylamino)propan-2-ol

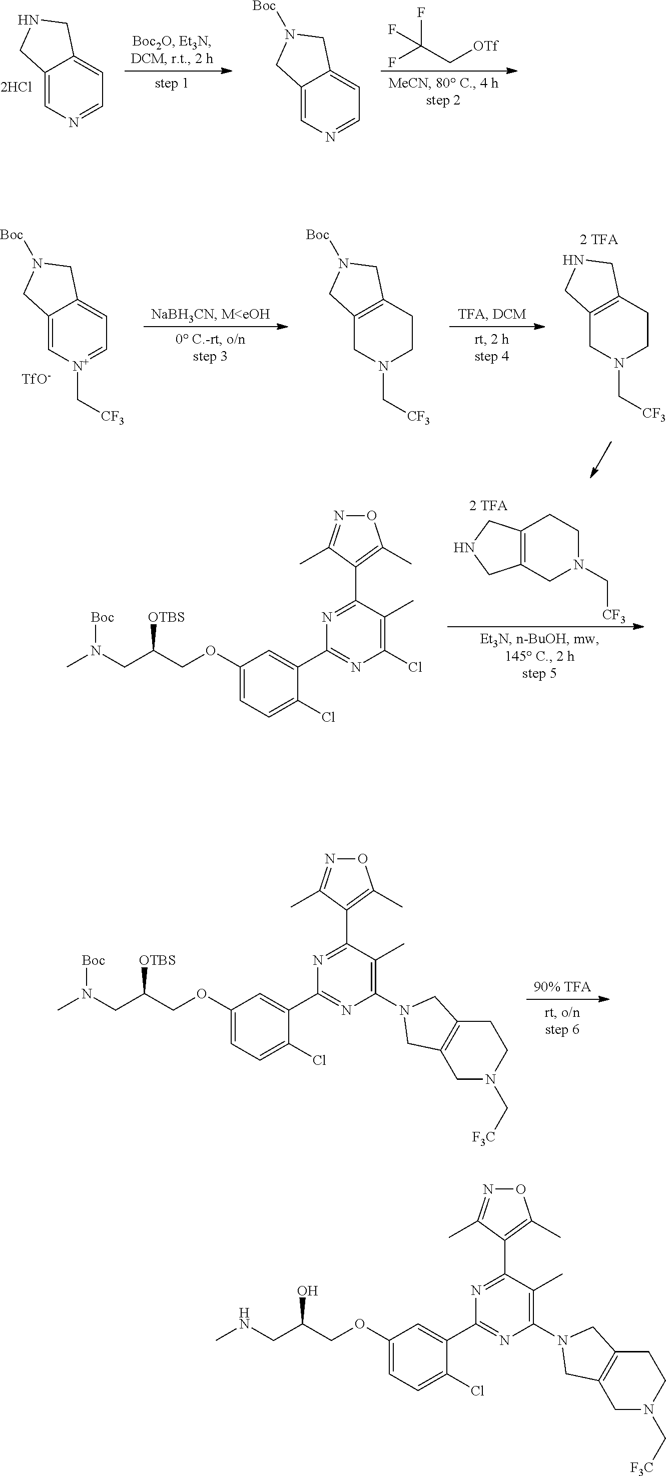

### Step 1: tert-butyl 1H-pyrrolo[3,4-c]pyridine-2(3H)-carboxylate

A solution of 2,3-dihydro-1H-pyrrolo[3,4-c]pyridine dihydrochloride (1.0 g, 5.2 mmol) and Et₃N (1.1 g, 10.9 mmol) in DCM (50 mL) was treated with slow addition of Boc₂O (1.2 g, 5.5 mmol). The mixture was stirred at room temperature for 2 h, concentrated in rotary evaporator and the residue was purified by column chromatography over silicagel (petroleum ether/EtOAc=1/1) to give tert-butyl 1H-pyrrolo[3,4-c]pyridine-2(3H)-carboxylate (0.9 g, 78% yield). ESI-LCMS (m/z): 221.1 found for [M+H]⁺.

### Step 2: 2-(tert-butoxycarbonyl)-5-(2,2,2-trifluoroethyl)-2,3-dihydro-1H-pyrrolo[3,4-c]pyridin-5-ium trifluoromethanesulfonate

A solution of tert-butyl 1H-pyrrolo[3,4-c]pyridine-2(3H)-carboxylate (500 mg, 2.3 mmol) in MeCN (10 mL) was treated with 2,2,2-trifluoroethyl trifluoromethane sulfonate (1.1 g, 4.7 mmol) and the reaction mixture was stirred at external temperature of 80° C. for 4 h. After being cooled down to room temperature, the mixture was concentrated to give 2-(tert-butoxycarbonyl)-5-(2,2,2-trifluoroethyl)-2,3-dihydro-1H-pyrrolo[3,4-c]pyridin-5-ium trifluoro methanesulfonate (0.7 g, crude), which was used directly without further purification. ESI-LCMS (m/z): 303.1 found for [M+H]⁺.

### Step 3: tert-butyl 5-(2,2,2-trifluoroethyl)-4,5,6,7-tetrahydro-1H-pyrrolo[3,4-c]pyridine-2(3H)-carboxylate

A solution of 2-(tert-butoxycarbonyl)-5-(2,2,2-trifluoroethyl)-2,3-dihydro-1H-pyrrolo[3,4-c]pyridin-5-ium trifluoromethanesulfonate (700 mg, crude from step 2) in MeOH (100 mL) was treated with NaBH₃CN (302 mg, 4.8 mmol) and the reaction mixture was stirred at room temperature for 16 h. and then concentrated under vacuum. The residue was dissolved in DCM (50 ml) and the solution was washed with water (50 ml). The organic layer was concentrated and the residue was purified by column chromatography over silicagel (petroleum ether/EtOAc=1/3) to give tert-butyl 5-(2,2,2-trifluoroethyl)-4,5,6,7-tetrahydro-1H-pyrrolo[3,4-c]pyridine-2(3H)-carboxylate (white solid, 350 mg, 50% yield for two steps). ESI-LCMS (m/z): 307.0 found for [M+H]⁺.

### Step 4: 5-(2,2,2-trifluoroethyl)-2,3,4,5,6,7-hexahydro-1H-pyrrolo[3,4-c]pyridine trifluoroacetate salt

A solution of tert-butyl 5-(2,2,2-trifluoroethyl)-4,5,6,7-tetrahydro-1H-pyrrolo[3,4-c]pyridine-2(3H)-carboxylate (200 mg, 0.65 mmol) in TFA/DCM (v/v=1/3, 10 ml) was stirred at room temperature for 2 h. and then the solvent was removed under vacuo to afford 5-(2,2,2-trifluoroethyl)-2,3,4,5,6,7-hexahydro-1H-pyrrolo[3,4-c]pyridine as trifluoroacetate salt, which was used directly without further purification. Assumed quantitative yield. ESI-LCMS (m/z): 207.1 found for [M+H]⁺.

### Step 5: tert-butyl (R)-2-(tert-butyldimethylsilyloxy)-3-(4-chloro-3-(4-(3,5-dimethylisoxazol-4-yl)-5-methyl-6-(5-(2,2,2-trifluoroethyl)-4,5,6,7-tetrahydro-1H-pyrrolo[3,4-c]pyridin-2(3H)-yl)pyrimidin-2-yl)phenoxy)propyl(methyl) carbamate

A reaction pressure vessel was charged with a mixture of tert-butyl (R)-2-(tert-butyl dimethylsilyloxy)-3-(4-chloro-3-(4-chloro-6-(3,5-dimethylisoxazol-4-yl)-5-methylpyrimidin-2-yl)phenoxy)propyl(methyl)carbamate (364 mmol, 0.56 mmol); 5-(2,2,2-trifluoroethyl)-2,3,4,5,6,7-hexahydro-1H-pyrrolo[3,4-c]pyridine TFA salt, (or any other suitably substituted primary or secondary amine, 0.65 mmol), Et₃N (226 mg, 2.24 mmol) and n-BuOH (5 mL). The vessel was capped, placed in a microwave reactor and irradiated for 2 h. at external temperature of 145° C. The solvent was then concentrated in vacuo and the residue was purified by preparative TLC to afford tert-butyl (R)-2-(tert-butyldimethylsilyloxy)-3-(4-chloro-3-(4-(3,5-dimethylisoxazol-4-yl)-5-methyl-6-(5-(2,2,2-trifluoroethyl)-4,5,6,7-tetrahydro-1H-pyrrolo[3,4-c]pyridin-2(3H)-yl)pyrimidin-2-yl)phenoxy)propyl(methyl)carbamate (450 mg, 98% yield). ESI-LCMS (m/z): 821.0 found for [M+H]⁺.

### Step 6: (2R)-1-(4-chloro-3-(4-(3,5-dimethylisoxazol-4-yl)-5-methyl-6-(5-(2,2,2-trifluoroethyl)-4,5,6,7-tetrahydro-1H-pyrrolo[3,4-c]pyridin-2(3H)-yl)pyrimidin-2-yl)phenoxy)-3-(methylamino)propan-2-ol

A solution of tert-butyl (R)-2-(tert-butyldimethylsilyloxy)-3-(4-chloro-3-(4-(3,5-dimethyl-isoxazol-4-yl)-5-methyl-6-(5-(2,2,2-trifluoroethyl)-4,5,6,7-tetrahydro-1H-pyrrolo[3,4-c]pyridin-2(3H)-yl)pyrimidin-2-yl)phenoxy)propyl(methyl)carbamate (150 mg, 0.18 mmol) in TFA/water (20:1 v/v, 10.5 mL) was stirred at room temperature for 16 h. The mixture was concentrated under vacuum, the residue was dissolved in MeOH (10 ml), and the solution was adjusted to pH 7-8 with ammonia. The mixture was concentrated, and the residue was purified by preparative HPLC to give (2R)-1-(4-chloro-3-(4-(3,5-dimethylisoxazol-4-yl)-5-methyl-6-(5-(2,2,2-trifluoroethyl)-4,5,6,7-tetrahydro-1H-pyrrolo[3,4-c]pyridin-2(3H)-yl)pyrimidin-2-yl) phenoxy)-3-(methyl amino)propan-2-ol (white solid, 38 mg, 35% yield). ESI-LCMS (m/z): 607.2 found for [M+H]; ¹HNMR (400 MHz, CDCl₃) δ ppm: 7.26 (d, J=8.8 Hz, 1H), 7.22 (d, J=3.2 Hz, 1H), 6.81 (dd, J=8.8 and 3.2 Hz, 1H), 4.50 (br s, 4H), 4.02-3.97 (m, 1H), 3.91 (d, J=5.2 Hz, 2H), 3.23 (s, 2H), 3.13-3.04 (m, 2H), 2.87-2.83 (m, 2H), 2.76-2.63 (m, 2H), 2.40 (s, 3H), 2.32 (s, 3H), 2.22 (s, 3H), 2.21 (s, 3H), 2.20-2.15 (m, 2H).

### Biological Assays

### General Materials

S-adenosylmethionine (SAM), S-adenosylhomocysteine (SAH), bicine, Tween20, dimethylsulfoxide (DMSO), bovine skin gelatin (BSG), sodium butyrate and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

### Substrates

Peptide representative of human histone H3 residues 16-30 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-PRKQLATKAARKSAP-amide and contained a monomethylated arginine at position 26 (SEQ ID NO.:1).

### Molecular Biology

Human CARM1 (PRMT4) (NM_199141.1) transcript clone was amplified from an HEK 293 cDNA library, incorporating a flanking 5′ sequence encoding a FLAG tag (MDYKDDDDK) (SEQ ID NO.:2) fused directly to Ala 2 of CARM1 and 3′ sequence encoding a hexa His sequence (EGHHHHHH) (SEQ ID NO.:3) fused directly to Ser 608. The gene sequence encoding isoform1 containing a deletion of amino acids 539-561 was amplified subsequently and subcloned into pFastBacMam (Viva Biotech).

### Protein Expression

Recombinant baculovirus were generated according to Bac-to-Bac kit instructions (Life Technologies). Protein over-expression was accomplished by infecting exponentially growing HEK 293F cell culture at 1.3×10⁶ cell/ml with virus (MOI=10) in the presence of 8 mM sodium butyrate. Infections were carried out at 37° C. for 48 hours, harvested by centrifugation, and stored at −80° C. for purification.

### Protein Purification

Expressed full-length human Flag- and His-tagged CARM1 protein was purified from cell paste by anti-flag M2 affinity chromatography with resin equilibrated with buffer containing 20 mM Tris, 150 mM NaCl, 5% glycerol, pH 7.8. Column was washed with 500 mM NaCl in buffer A and Flag-CARM1-His was eluted with 200 ug/ml FLAG peptide in buffer A. Pooled fractions were dialyzed in 20 mM Tris, 150 mM NaCl, 5% glycerol and 1 mM DTT, pH 7.8. The purity of recovered protein was 94.

### Predicted Translations

Flag-CARM1-His
(SEQ ID NO.: 4)
MDYKDDDDKAAAAAAVGPGAGGAGSAVPGGAGPCATVSVFPGARLLTI
GDANGEIQRHAEQQALRLEVRAGPDSAGIALYSHEDVCVFKCSVSRET
ECSRVGKQSFIITLGCNSVLIQFATPNDFCSFYNILKTCRGHTLERSV
FSERTEESSAVQYFQFYGYLSQQQNMMQDYVRTGTYQRAILQNHTDFK
DKIVLDVGCGSGILSFFAAQAGARKIYAVEASTMAQHAEVLVKSNNLT
DRIVVIPGKVEEVSLPEQVDIIISEPMGYMLFNERMLESYLHAKKYLK
PSGNMFPTIGDVHLAPFTDEQLYMEQFTKANFWYQPSFHGVDLSALRG
AAVDEYFRQPVVDTFDIRILMAKSVKYTVNFLEAKEGDLHRIEIPFKF
HMLHSGLVHGLAFWFDVAFIGSIMTVWLSTAPTEPLTHWYQVRCLFQS
PLFAKAGDTLSGTCLLIANKRQSYDISIVAQVDQTGSKSSNLLDLKNP
FFRYTGTTPSPPPGSHYTSPSENMWNTGSTYNLSSGMAVAGMPTAYDL
SSVIASGSSVGHNNLIPLGSSGAQGSGGGSTSAHYAVNSQFTMGGPAI
SMASPMSIPTNTMHYGSEGHHHHHH

### General Procedure for CARM1 Enzyme Assays on Peptide Substrates

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of CARM1, was added to columns 11, 12, 23, 24, rows I-P for the minimum signal control. A cocktail (40 ul) containing the CARM1 enzyme was added by Multidrop Combi (Thermo-Fisher). The compounds were allowed to incubate with CARM1 for 30 min at room temperature, then a cocktail (10 ul) containing ³H-SAM and peptide was added to initiate the reaction (final volume=51 ul). The final concentrations of the components were as follows: CARM1 was 0.25 nM, ³H-SAM was 30 nM, peptide was 250 nM, SAH in the minimum signal control wells was 1 mM, and the DMSO concentration was 2%. The assays were stopped by the addition of non-radiolabeled SAM (10 ul) to a final concentration of 300 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

where dpm=disintegrations per minute, cmpd=signal in assay well, and min and max are the respective minimum and maximum signal controls.

$$parameter IC 50 fit$$

$$Y = Bottom + \frac{( Top - Bottom )}{( 1 + {( \frac{X}{{IC}_{50}} )}^{Hill Coefficient}}$$

where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### RKO Methylation Assay

RKO adherent cells were purchased from ATCC (American Type Culture Collection), Manassas, Va., USA. DMEM/Glutamax medium, penicillin-streptomycin, heat inactivated fetal bovine serum, 0.05% trypsin and D-PBS were purchased from Life Technologies, Grand Island, N.Y., USA. Odyssey blocking buffer, 800CW goat anti-rabbit IgG (H+L) antibody, and Licor Odyssey infrared scanner were purchased from Licor Biosciences, Lincoln, Nebr., USA. Asymmetric di-methyl PABP1 antibody was purchased from Cell Signaling Technology, Danvers, Mass., USA. Methanol was purchased from VWR, Franklin, Mass., USA. 10% Tween 20 was purchased from KPL, Inc., Gaithersburg, Md., USA. Paraformaldehyde (PFA) was purchased from EM Sciences. DRAQ5 was purchased from Biostatus Limited, Leicestershire, UK.

RKO adherent cells were maintained in growth medium (DMEM/Glutamax medium supplemented with 10% v/v heat inactivated fetal bovine serum and 100 units/mL penicillin-streptomycin) and cultured at 37° C. under 5% CO₂.

Cell treatment, In Cell Western (ICW) for detection of asymmetric di-methyl PABP1 and DNA content: RKO cells were seeded in assay medium at a concentration of 30,000 cells per mL to a poly-D-lysine coated 384 well culture plate (BD Biosciences 356697) with 50 μL per well. Compound (100 nL) from a 96-well source plate was added directly to 384 well cell plate. Plates were incubated at 37° C., 5% CO₂ for 48 hours. After two days of incubation, plates were brought to room temperature outside of the incubator for ten minutes and blotted on paper towels to remove cell media. Cells were fixed for 20 minutes at room temperature by adding 50 ul of 8% PFA followed by aspiration of supernatant with the Biotek EL406 plate washer. Cells were then permeabilized by addition of 50 μL of ice cold 100% methanol directly to each well and incubated for 30 min at room temperature. After 30 min, plates were transferred to a Biotek EL406 plate washer and washed 2 times with 100 μL per well of wash buffer (1×PBS). Next 60 μL per well of Odyssey blocking buffer (Odyssey Buffer with 0.1% Tween 20 (v/v)) were added to each plate and incubated 1 hour at room temperature. Blocking buffer was removed and 20 μL per well of primary antibody was added (asymmetric-methyl PABP1) diluted 1:400 in Odyssey buffer with 0.1% Tween 20 (v/v)) and plates were incubated overnight (16 hours) at 4° C. Plates were washed 5 times with 100 μL per well of wash buffer. Next 20 μL per well of secondary antibody was added (1:800 800CW goat anti-rabbit IgG (H+L) antibody, 1:2000 DRAQ5 in Odyssey buffer with 0.1% Tween 20 (v/v)) and incubated for 1 hour at room temperature. The plates were washed 5 times with 100 μL per well wash buffer then 2 times with 100 μL per well of water. Plates were allowed to dry at room temperature then imaged on the Licor Odyssey machine which measures integrated intensity at 700 nm and 800 nm wavelengths. Both 700 and 800 channels were scanned.

Calculations.

First, the ratio for each well was determined by:

$$( \frac{asymmetric di \text{-} methyl PABP 1 \; 800 nm value}{DRAQ 5 \; 700 nm value} )$$

Each plate included fourteen control wells of DMSO only treatment (minimum inhibition) as well as fourteen control wells for maximum inhibition treated with 20 μM of a reference compound. The average of the ratio values for each control type was calculated and used to determine the percent activation for each test well in the plate. Reference compound was serially diluted three-fold in DMSO for a total of nine test concentrations, beginning at 20 μM.

Percent inhibition was determined and IC₅₀ curves were generated using triplicate wells per concentration of compound.

$$Percent Inhibition = 100 - ( ( \frac{\begin{matrix}( Minimum Inhibition Ratio ) - \\ ( Individual Test Sample Ratio )\end{matrix}}{\begin{matrix}( Minimum Inhibition Ratio ) - \\ ( Maximum Inhibition Ratio )\end{matrix}} ) * 100 )$$

### Other Embodiments

The foregoing has been a description of certain non-limiting embodiments of the invention. Those of ordinary skill in the art will appreciate that various changes and modifications to this description may be made without departing from the spirit or scope of the present invention, as defined in the following claims.

1. A compound of Formula (I): 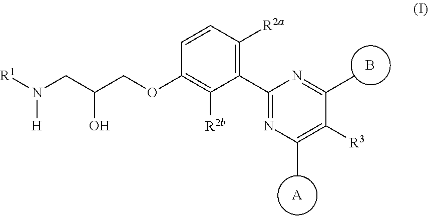
or pharmaceutically acceptable salt thereof;
wherein:
R¹ is hydrogen, —CHO, or unsubstituted C₁₋₃alkyl;
each instance of R^{2a} and R^{2b} is independently hydrogen, halogen, unsubstituted C₁₋₃alkyl, or C₁₋₃haloalkyl;
R³ is unsubstituted C₁₋₃alkyl, C₁₋₃haloalkyl, or halogen;
Ring A is of formula (A-i), (A-ii), or (A-iii): 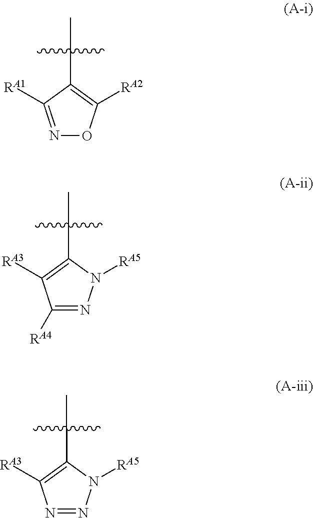
wherein:
each instance of R^{A1} and R^{A2} is independently unsubstituted C₁₋₃alkyl, Ci-3haloalkyl, or unsubstituted cyclopropyl;
R^{A3} is unsubstituted C₁₋₃alkyl, C₁₋₃haloalkyl, halogen, or —CN;
R^{A4} is hydrogen, unsubstituted C₁₋₃alkyl, C₁₋₃haloalkyl, halogen, or —CN; and
R^{A5} is unsubstituted C₁₋₃alkyl or C₁₋₃haloalkyl;
Ring B is any one of formula (i) to (xxviii): 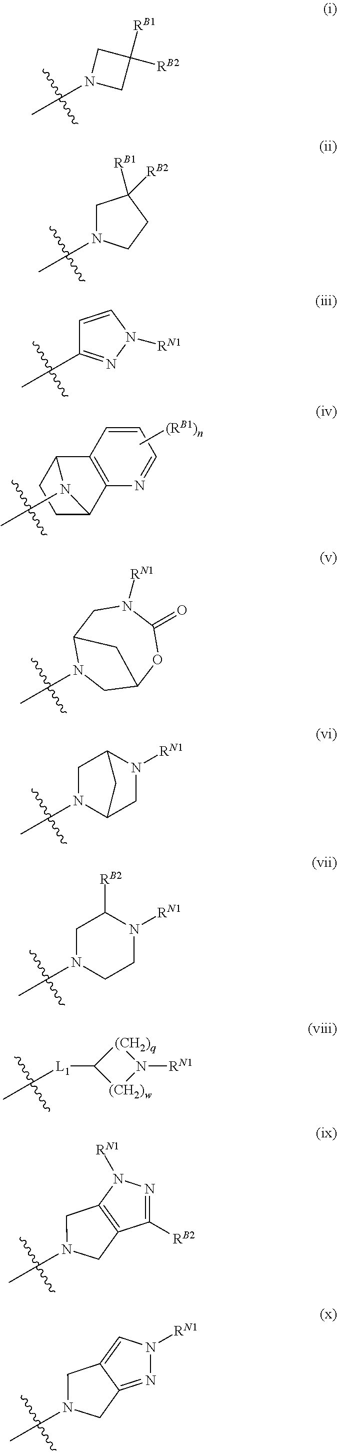 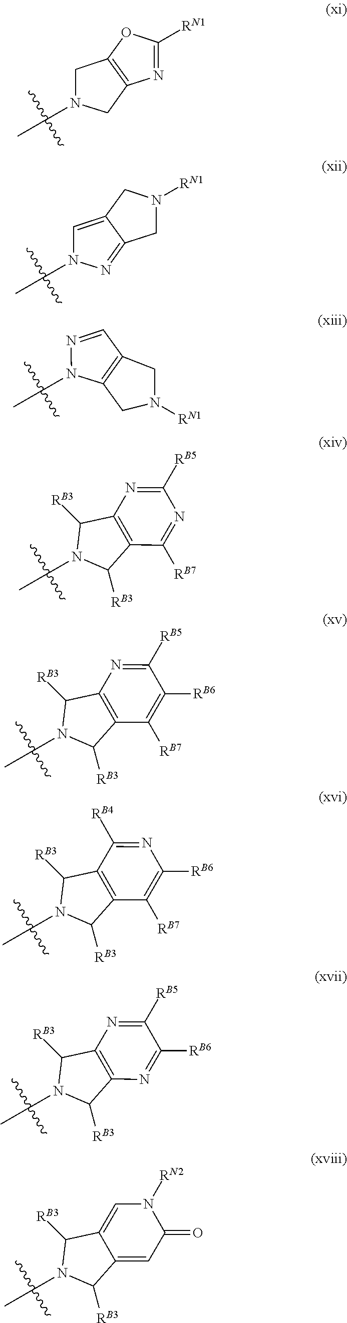 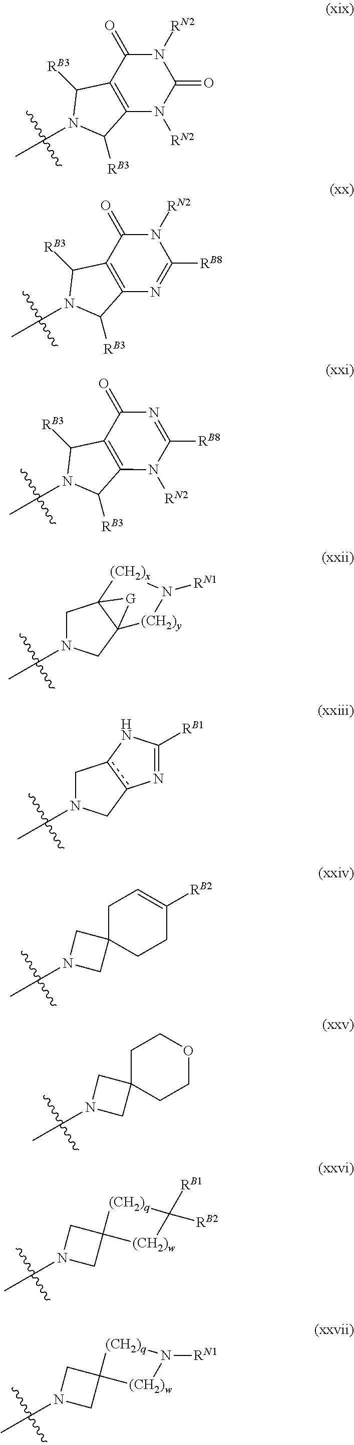 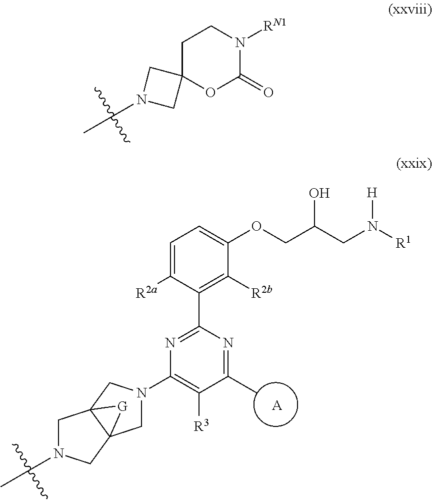
wherein:
q is 1, 2, or 3 and w is 1; or q is 2 and w is 0 or 2;
x is 1 and y is 1 or 2;
n is 0, 1, or 2;
L₁ is —NH—, substituted or unsubstituted C₂alkylene, substituted or unsubstituted C₂alkenylene, or substituted or unsubstituted C₂alkynylene;
R^{N1} is substituted or unsubstituted C₁₋₃alkyl, C₁₋₃haloalkyl substituted or unsubstituted C₃₋₆ carbocyclyl, substituted or unsubstituted 4- to 6-membered heterocyclyl, —C(═O)R^{N1A}, —C(═O)N(R^{N1A})(R^{N1B}), —C(═O)OR^{N1A}, or —S(O)₂R^{N1A};
wherein:
R^{N1A} is substituted or unsubstituted C₁₋₃alkyl, C₁₋₃haloalkyl, substituted or unsubstituted C₃₋₆ carbocyclyl, or substituted or unsubstituted 4- to 6-membered heterocyclyl;
R^{N1B} is hydrogen, substituted or unsubstituted C₁₋₃alkyl, C₁₋₃haloalkyl, substituted or unsubstituted C₃₋₆ carbocyclyl, or substituted or unsubstituted 4- to 6-membered heterocyclyl; or
R^{N1A} and R^{N1B} are joined to form a substituted or unsubstituted 4- to 6-membered heterocyclyl; or
each instance of R^{N2} and R^{B8} is independently substituted or unsubstituted C₁₋₃alkyl or C₁₋₃haloalkyl, or R^{N2} and R^{B8} are joined to form a substituted or unsubstituted 5- to 6-membered ring;
R^{B1} is substituted or unsubstituted C₁₋₃alkyl, C₁₋₃haloalkyl, halogen, —CN, —OR^{B1B}, —SR^{B1B}, —N(R^{B1A})(R^{B1B}), substituted or unsubstituted C₃₋₆ carbocyclyl, substituted or unsubstituted 4- to 6-membered heterocyclyl, —C(═O)R^{B1A}, —C(═O)N(R^{B1A})(R^{B1B}), —C(═O)OR^{B1A}, —S(O)₂R^{B1A}, —OC(═O)R^{B1A}, —OC(═O)N(R^{B1A})(R^{B1B}), —OC(═O)OR^{B1A}, —NR^{B1B}C(═O)R^{B1A}, —NR^{B1B}C(═O)N(R^{B1A})(R^{B1B}), or —NR^{B1B}C(═O)OR^{B1A};
wherein:
R^{B1A} is substituted or unsubstituted C₁₋₃alkyl, C₁₋₃haloalkyl, substituted or unsubstituted C₃₋₆ carbocyclyl, or substituted or unsubstituted 4- to 6-membered heterocyclyl; and
R^{B1B} is hydrogen, substituted or unsubstituted C₁₋₃alkyl, C₁₋₃haloalkyl, substituted or unsubstituted C₃₋₆ carbocyclyl, or substituted or unsubstituted 4- to 6-membered heterocyclyl; or
R^{B1A} and R^{B1B} are joined to form a substituted or unsubstituted 4- to 6-membered heterocyclyl;
R^{B2} is hydrogen, halogen, —OR^{B2A}, substituted or unsubstituted C₁₋₃alkyl, or Ci-3haloalkyl, wherein R^{B2A} is substituted or unsubstituted C₁₋₃alkyl or C₁₋₃haloalkyl; or
R^{B1} and R^{B2} are joined to form a substituted or unsubstituted 4- to 6-membered heterocyclyl;
each instance of R^{B3} is independently hydrogen, unsubstituted C₁₋₃alkyl, or Ci-3haloalkyl, provided at least one instance of R^{B3} is hydrogen;
each instance of R^{B4}, R^{B5}, R^{B6}, and R^{B7} is independently hydrogen, substituted or unsubstituted C₁₋₃alkyl, C₁₋₃haloalkyl, halogen, —CN, —OR^{B4B}, —SR^{B4B}, —N(R^{B4A})(R^{B4B}), substituted or unsubstituted C₃₋₆ carbocyclyl, substituted or unsubstituted 4- to 6-membered heterocyclyl, —C(═O)R^{B4A}, —C(═O)N(R^{B4A})(R^{B4B}), —C(═O)OR^{B4A}, —S(O)₂R^{B4A}, —OC(═O)R^{B4A}, —OC(═O)N(R^{B4A})(R^{B4B}), —OC(═O)OR^{B4A}, —NR^{B4B}C(═O)R^{B4A}, —NR^{B4B}C(═O)N(R^{B4A})(R^{B4B}), Or —NR^{B4B}C(═O)OR^{B4A};
wherein:
R^{B4A} is substituted or unsubstituted C₁₋₃alkyl, C₁₋₃haloalkyl, substituted or unsubstituted C₃₋₆ carbocyclyl, or substituted or unsubstituted 4- to 6-membered heterocyclyl; and
R^{B4B} is hydrogen, substituted or unsubstituted C₁₋₃alkyl, C₁₋₃haloalkyl, substituted or unsubstituted C₃₋₆ carbocyclyl, or substituted or unsubstituted 4- to 6-membered heterocyclyl; or
R^{B4A} and R^{B4B} are joined to form a substituted or unsubstituted 4- to 6-membered heterocyclyl;
and
wherein [symbol] represents a single or double bond; and
further wherein 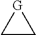
represents a single or double bond or G is —CH₂—;
wherein each instance of substituted independently refers to substitution with 1, 2, or 3 R^{C1} groups, as valency permits,
and wherein:
each instance of R^{C1} is independently unsubstituted C₁₋₃alkyl, C₁₋₃haloalkyl, halogen, —CN, —OR^{C1B}, —SR^{C1B}, —N(R^{C1A})(R^{C1B}), —C(═O)R^{C1A}, —C(═O)N(R^{C1A})(R^{C1B}), —C(═O)OR^{C1A}, —S(O)₂R^{C1A}, —OC(═O)R^{C1A}, —OC(═O)N(R^{C1A})(R^{C1B}), —OC(═O)OR^{C1A}, —NR^{C1B}C(═O)R^{C1A}, —NR^{C1B}C(═O)N(R^{C1A})(R^{C1B}), Or —NR^{C1B}C(═O)OR^{C1A};
wherein:
R^{C1A} is unsubstituted C₁₋₃alkyl, C₁₋₃haloalkyl, C₃₋₆carbocylyl unsubstituted or substituted with 1 or 2 R^{D1} groups; or 4-6 membered heterocyclyl unsubstituted or substituted with 1 or 2 R^{D1} groups; and
R^{C1B} is hydrogen, unsubstituted C₁₋₃alkyl, C₁₋₃haloalkyl, C₃₋₆carbocylyl unsubstituted or substituted with 1 or 2 R^{D1} groups, or 4-6 membered heterocyclyl unsubstituted or substituted with 1 or 2 R^{D1} groups; or
R^{C1A} and R^{C1B} are joined to form an 4- to 6-membered heterocyclyl unsubstituted or substituted with 1 or 2 R^{D1} groups; and
wherein:
each instance of R^{D1} is independently halogen, —CN, —OR^{D1A}, unsubstituted C₁₋₃alkyl, or C₁₋₃haloalkyl, wherein R^{D1A} is hydrogen, unsubstituted C₁₋₃alkyl, or C₁₋₃haloalkyl.

2-18. (canceled)

19. The compound or pharmaceutically acceptable salt of claim 1, wherein Ring A is of formula (A-i): 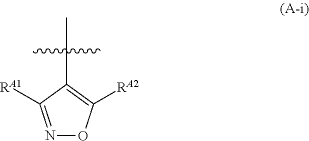
wherein at least one of R^{A1} and R^{A2} is —CH₃ or —CH₂CH₃.

20-21. (canceled)

22. The compound or pharmaceutically acceptable salt of claim 19, wherein Ring A is:

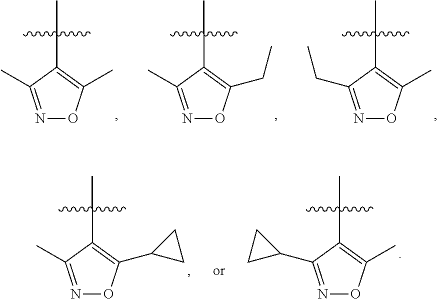

23-95. (canceled)

96. The compound or pharmaceutically acceptable salt of claim 1, wherein Ring B is of formula:

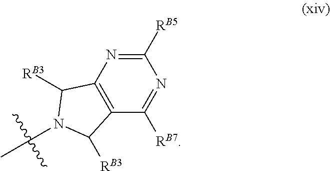

97. The compound or pharmaceutically acceptable salt of claim 96, wherein Ring B is of formula:

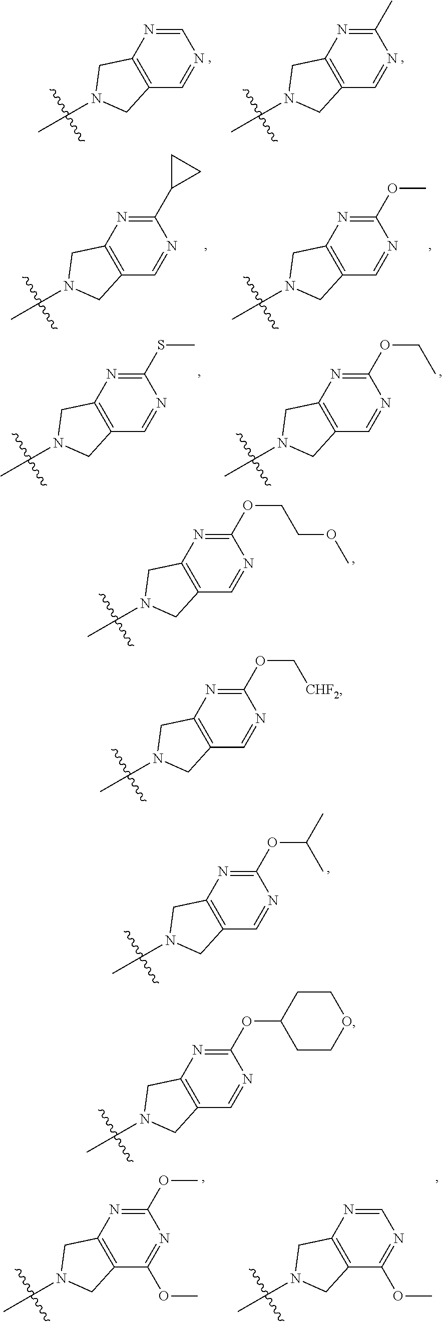

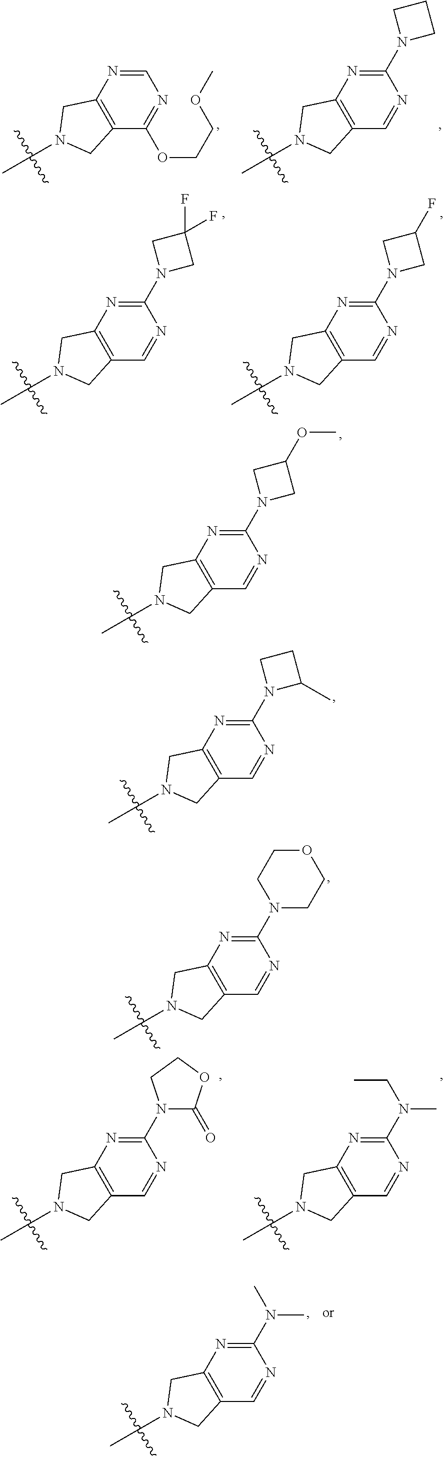

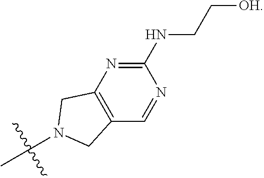 [SMILES: *N1Cc2cnc(NCCO)nc2C1]

98. The compound or pharmaceutically acceptable salt of claim 1, wherein Ring B is of formula:

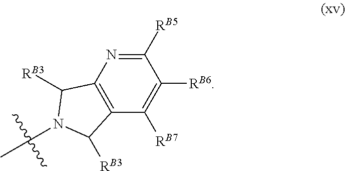

99. The compound or pharmaceutically acceptable salt of claim 98, wherein Ring B is of formula:

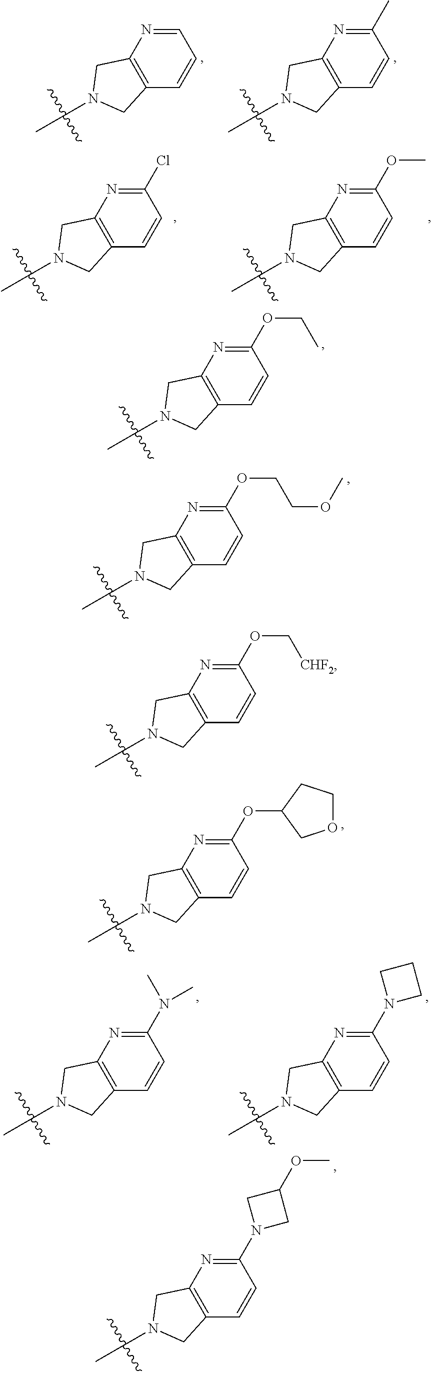

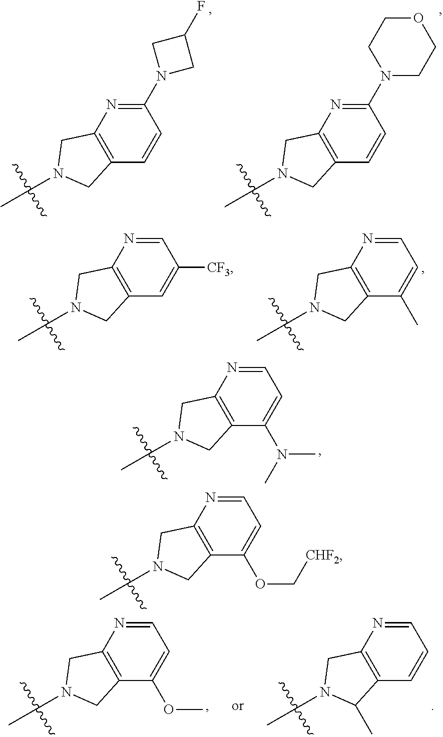

100. The compound or pharmaceutically acceptable salt of claim 1, wherein Ring B is of formula:

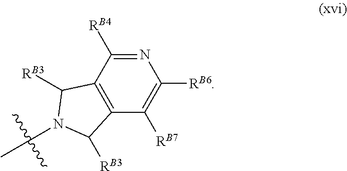

101. The compound or pharmaceutically acceptable salt of claim 100, wherein Ring B is of formula:

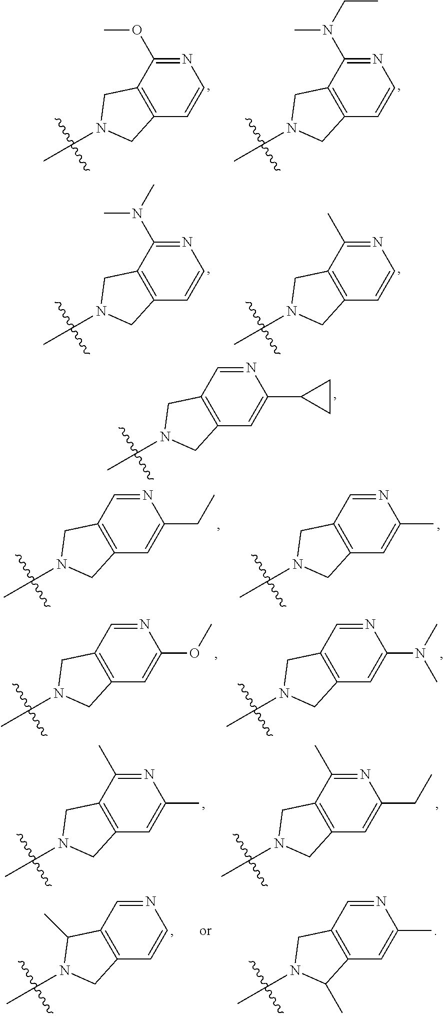

102. The compound or pharmaceutically acceptable salt of claim 1, wherein Ring B is of formula:

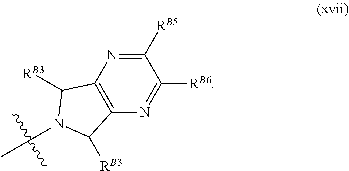

103. The compound or pharmaceutically acceptable salt of claim 102, wherein Ring B is of formula:

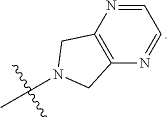 [SMILES: *N1Cc2nccnc2C1]

104-142. (canceled)

143. The compound of claim 1, wherein the compound is of Formula: 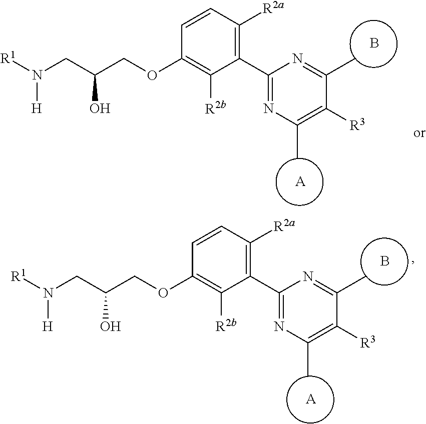
or a pharmaceutically acceptable salt thereof.

144. The compound of claim 1, wherein the compound is of Formula: 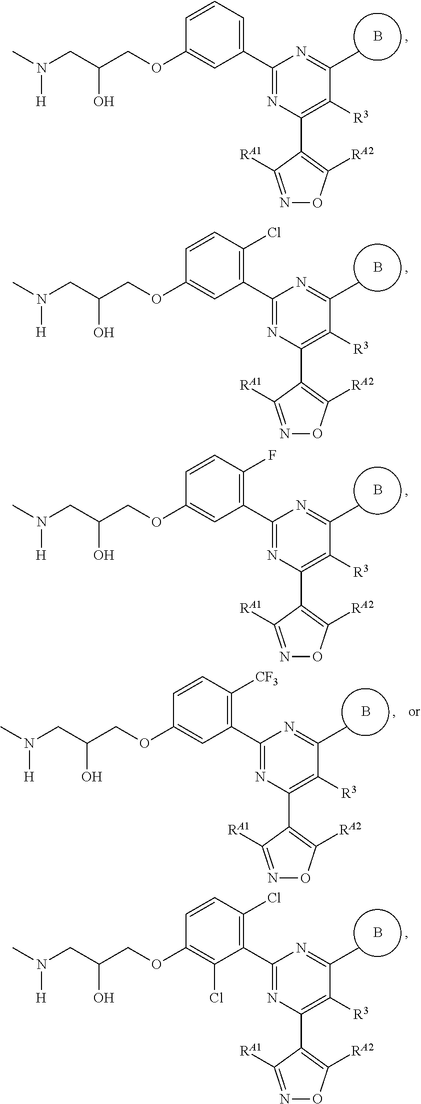
or a pharmaceutically acceptable salt thereof.

145-147. (canceled)

148. A pharmaceutical composition comprising the compound of claim 1 or a pharmaceutically acceptable salt thereof, and a pharmaceutically acceptable excipient.

149-159. (canceled)

160. The compound or pharmaceutically acceptable salt of claim 19, wherein Ring B is of formula:

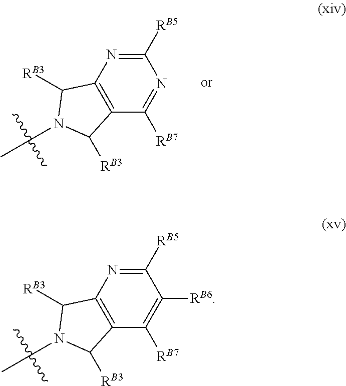

161. The compound of claim 1 which is: 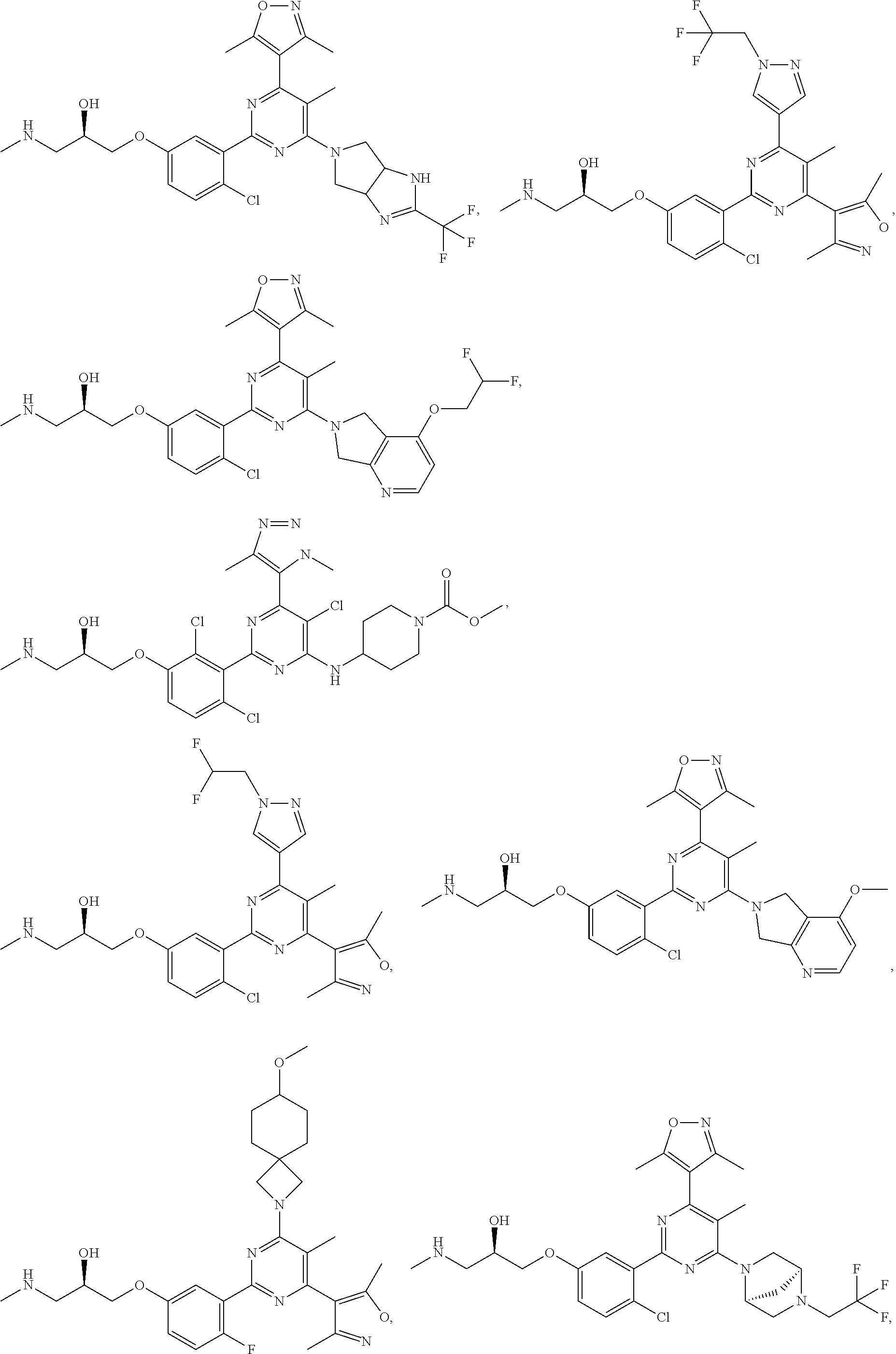 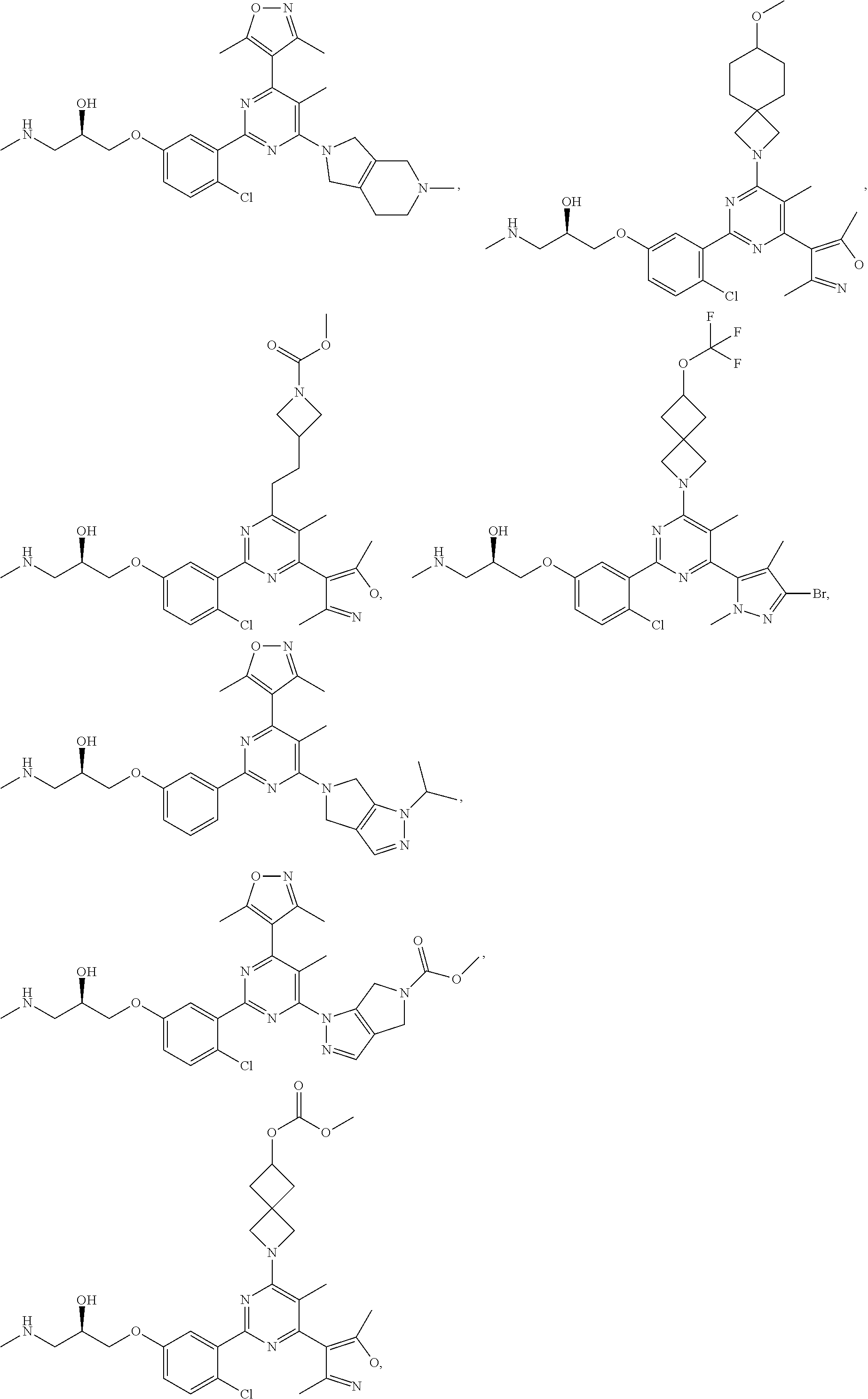 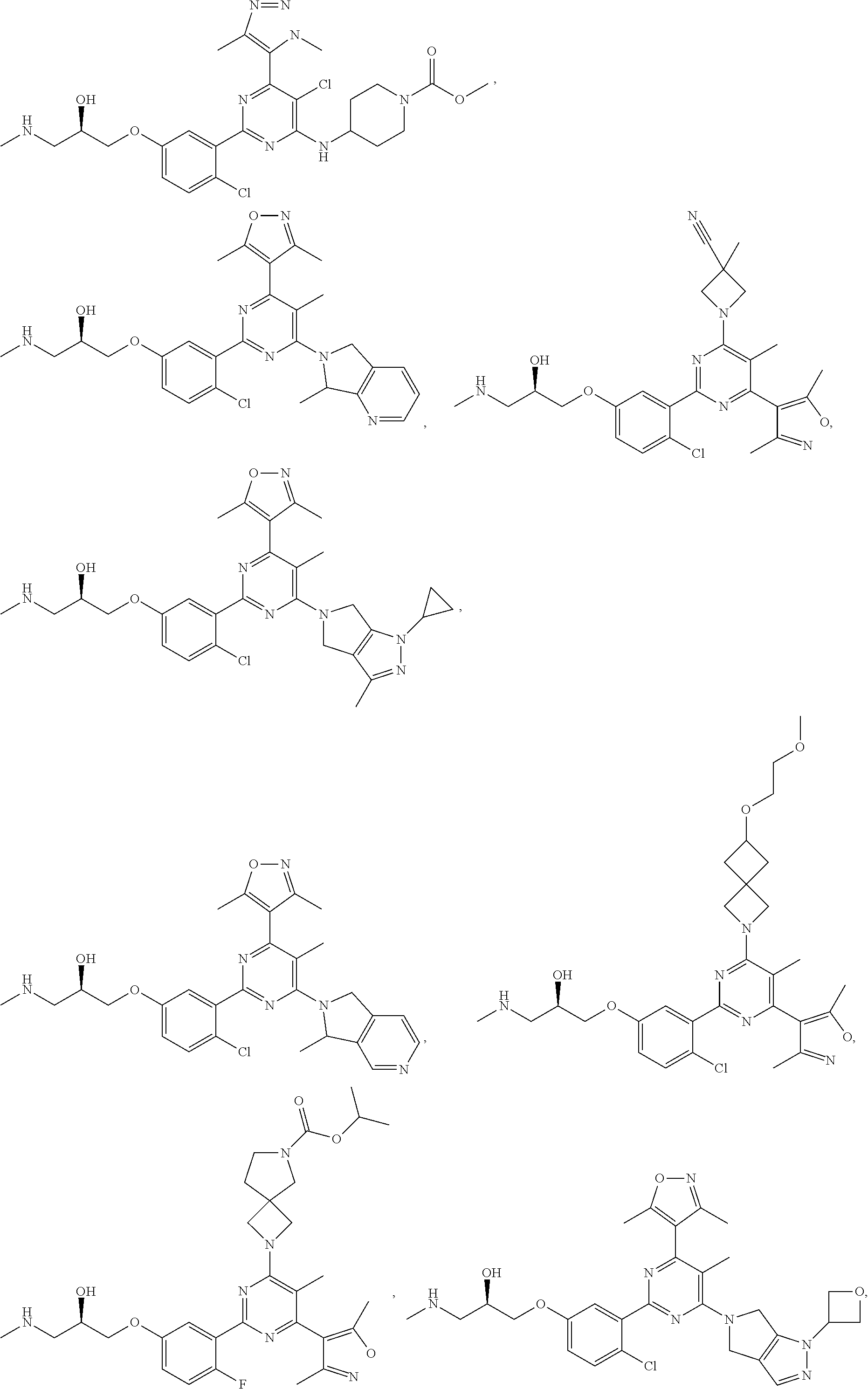 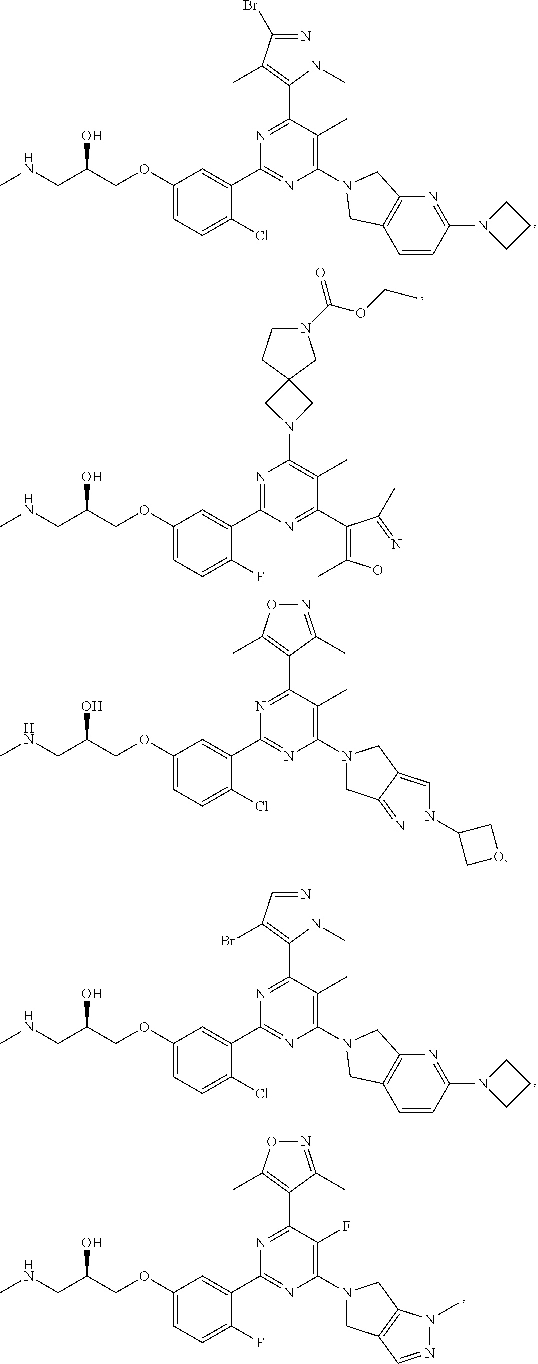 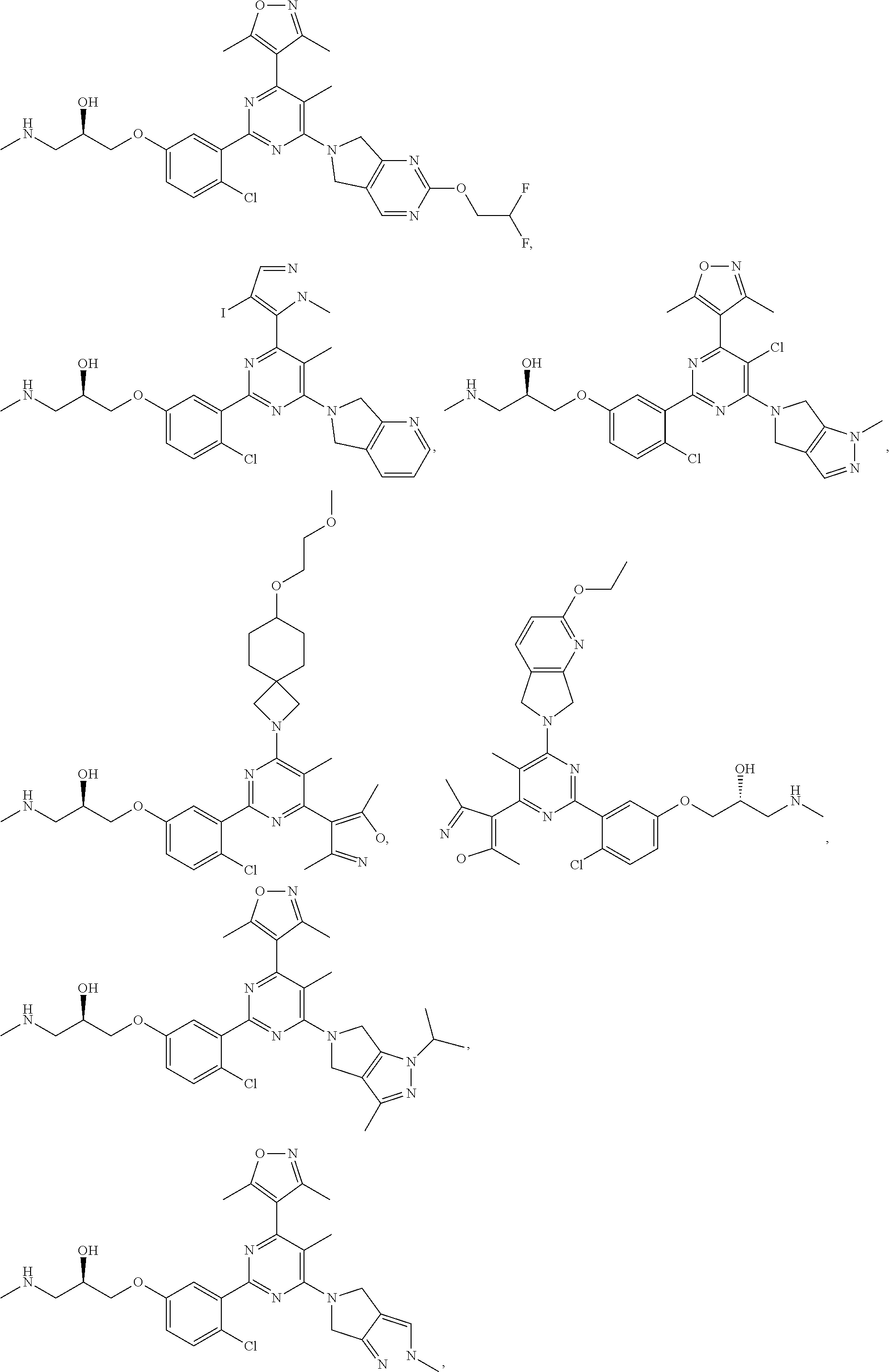 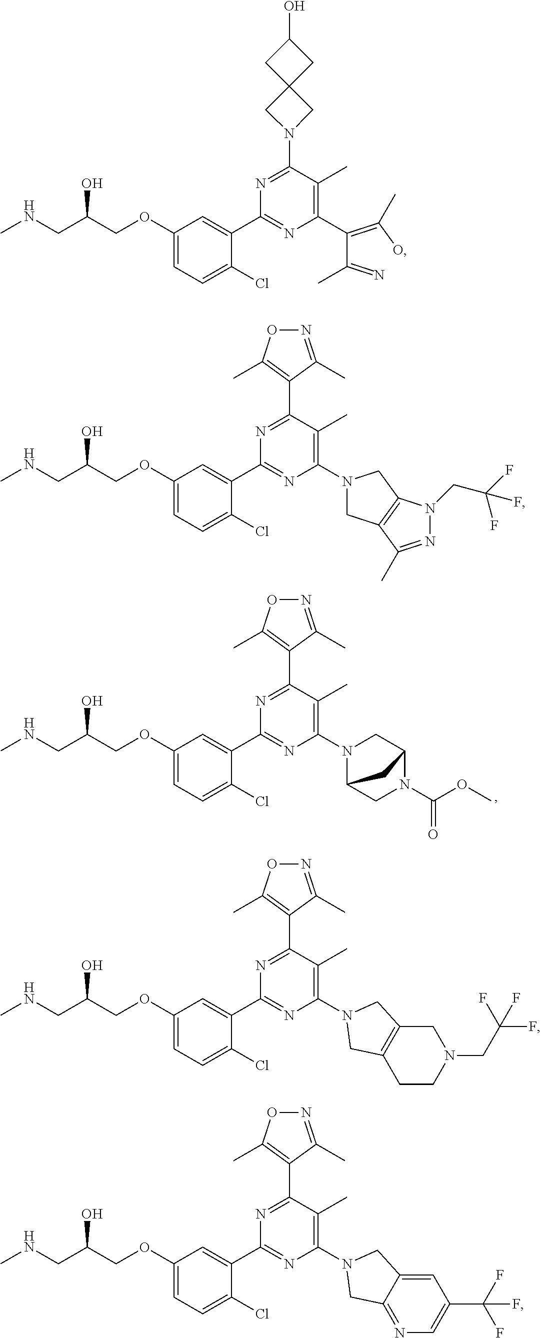 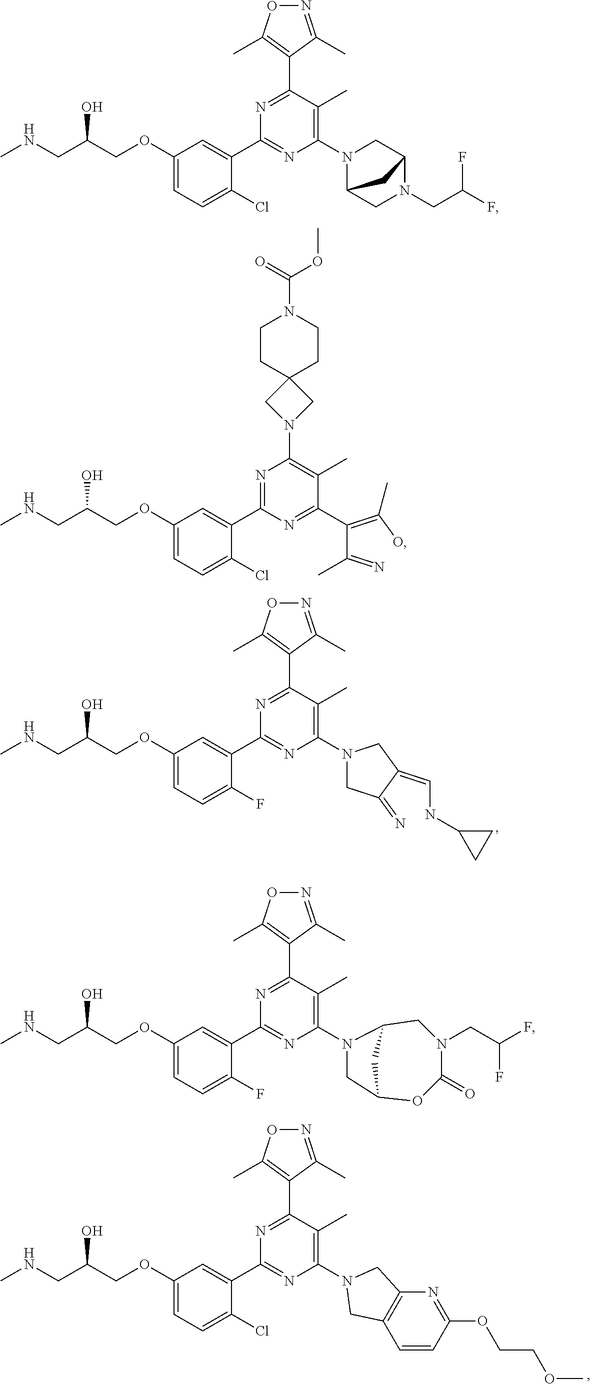 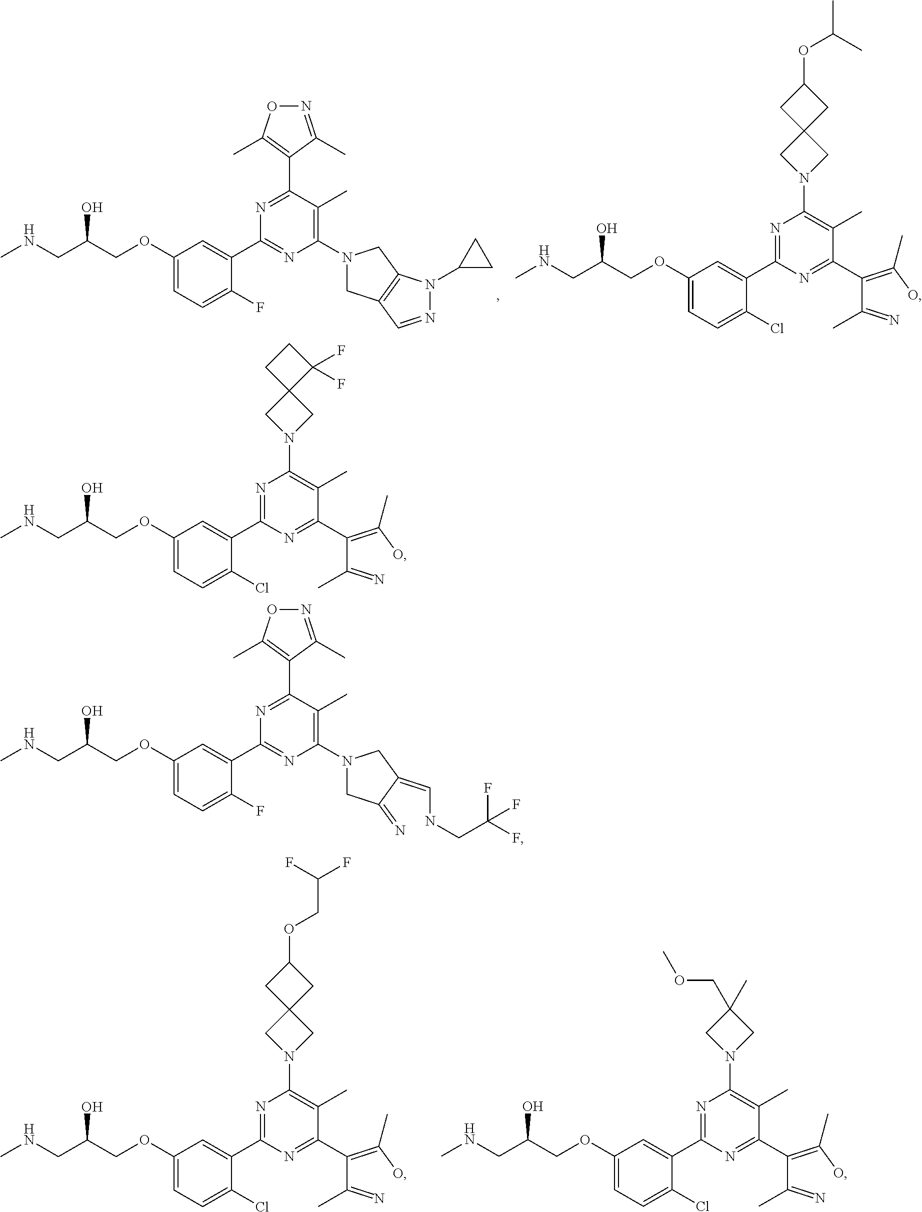 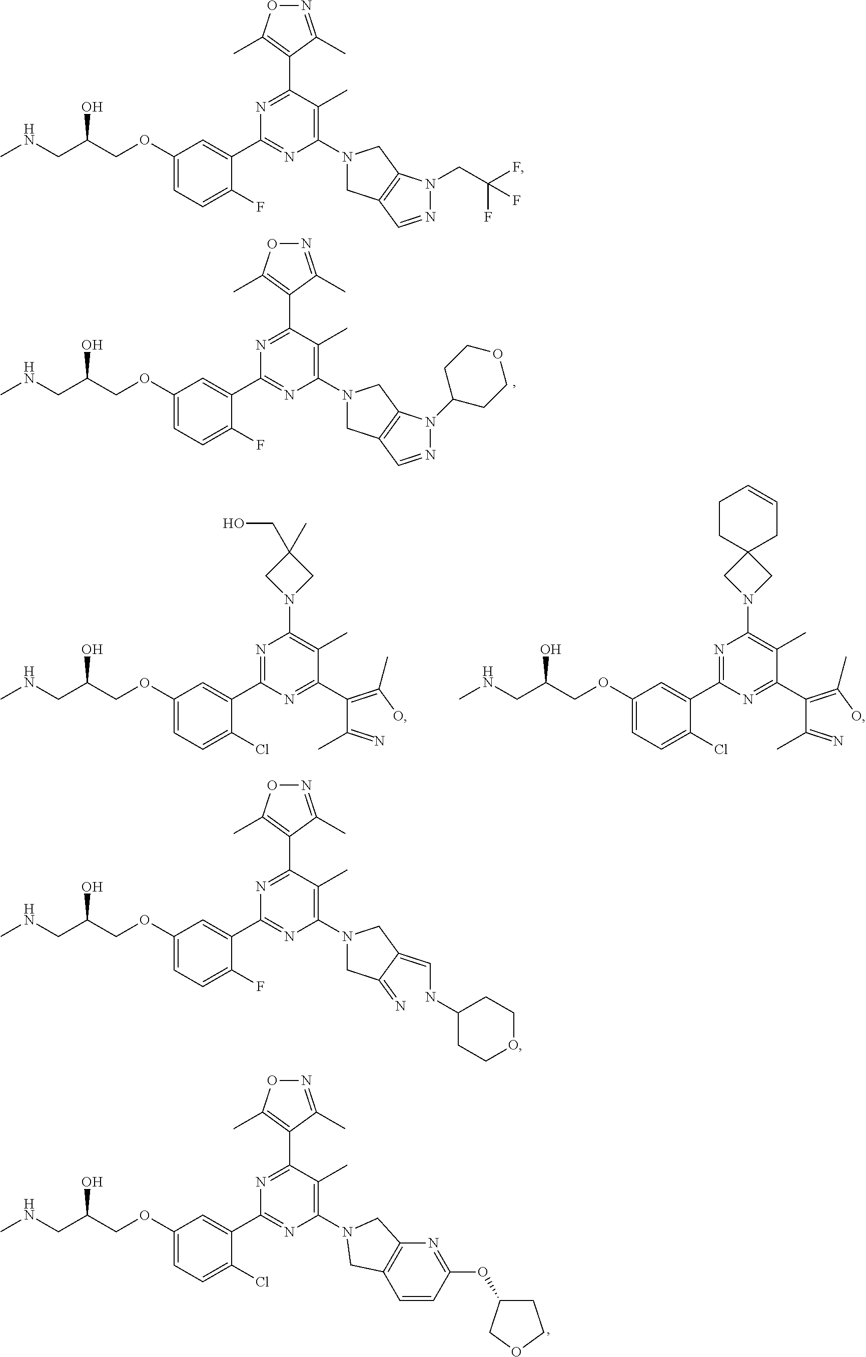 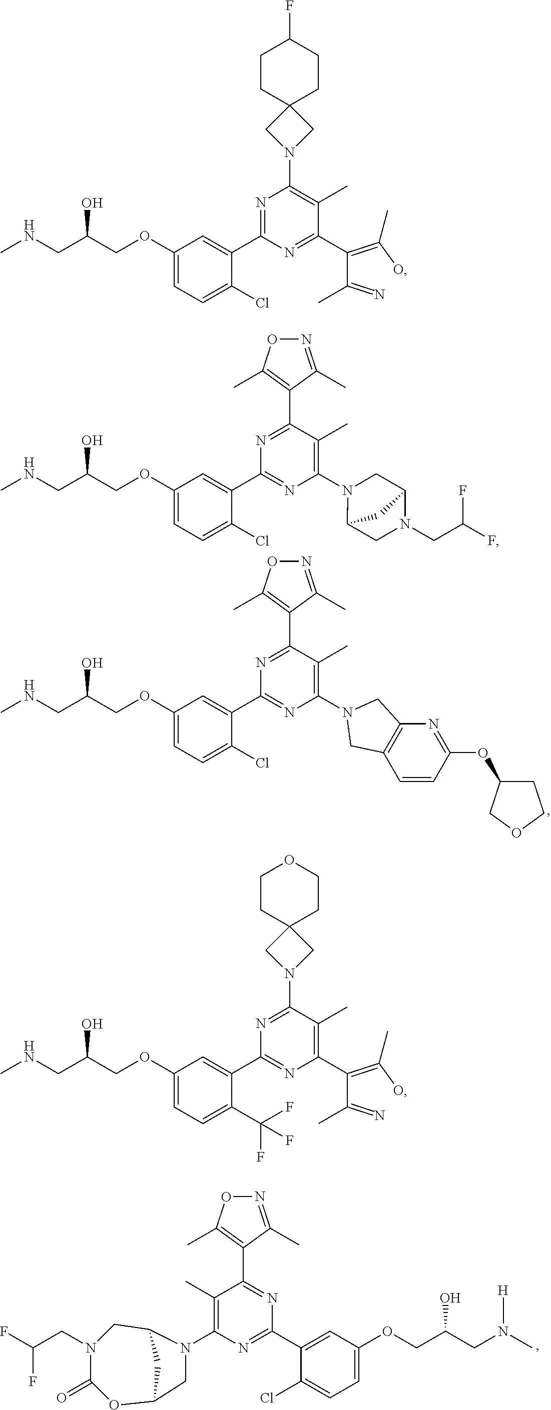 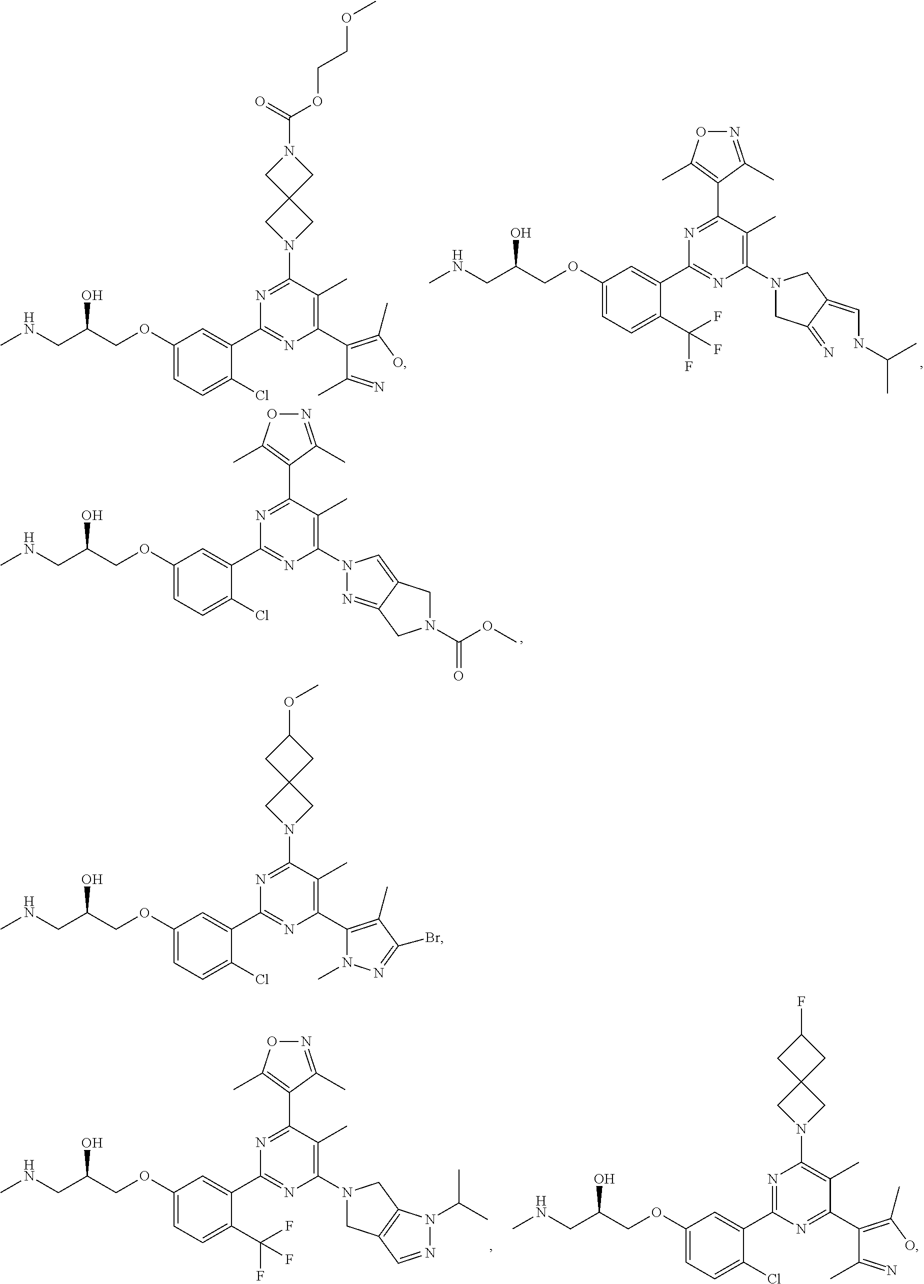 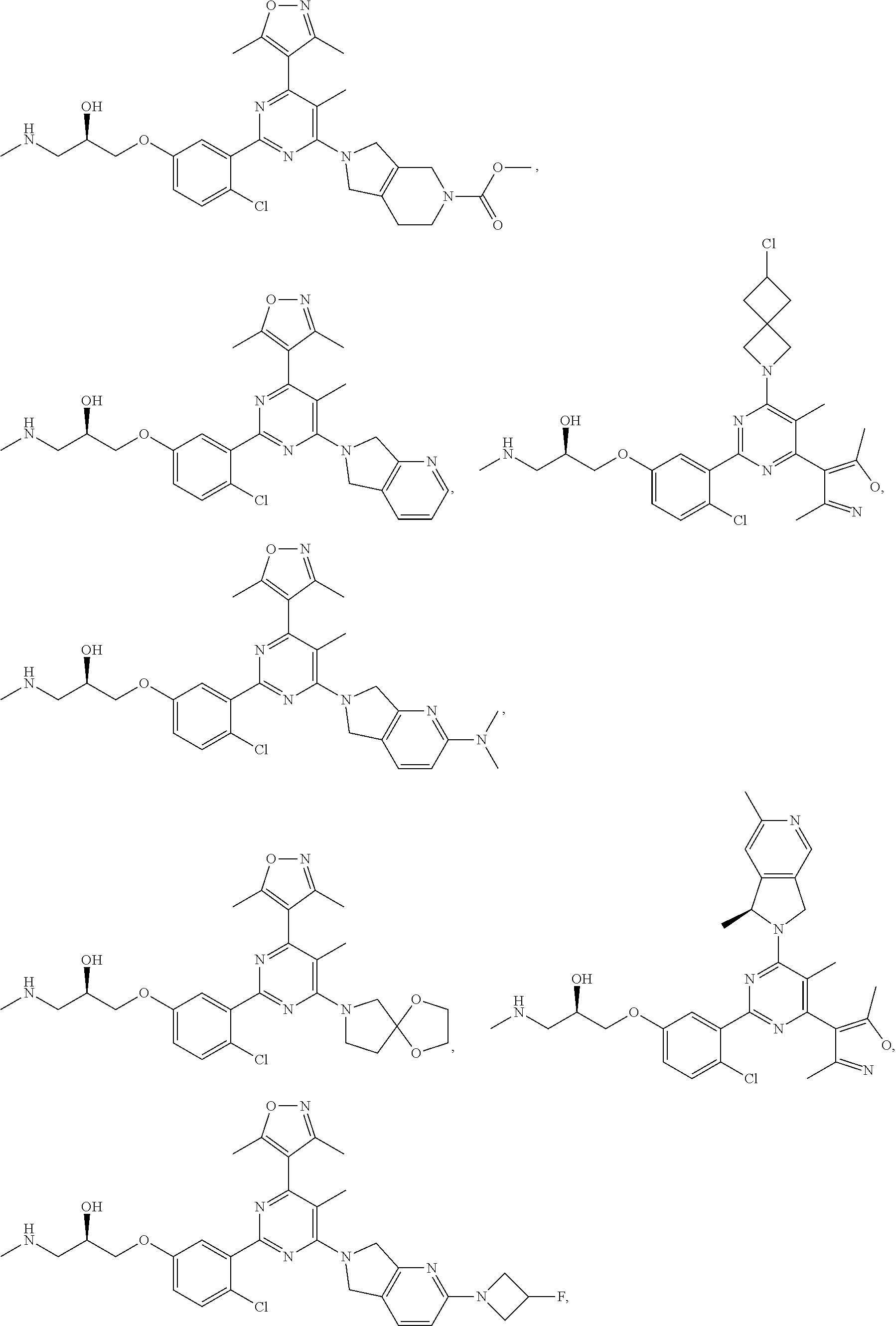 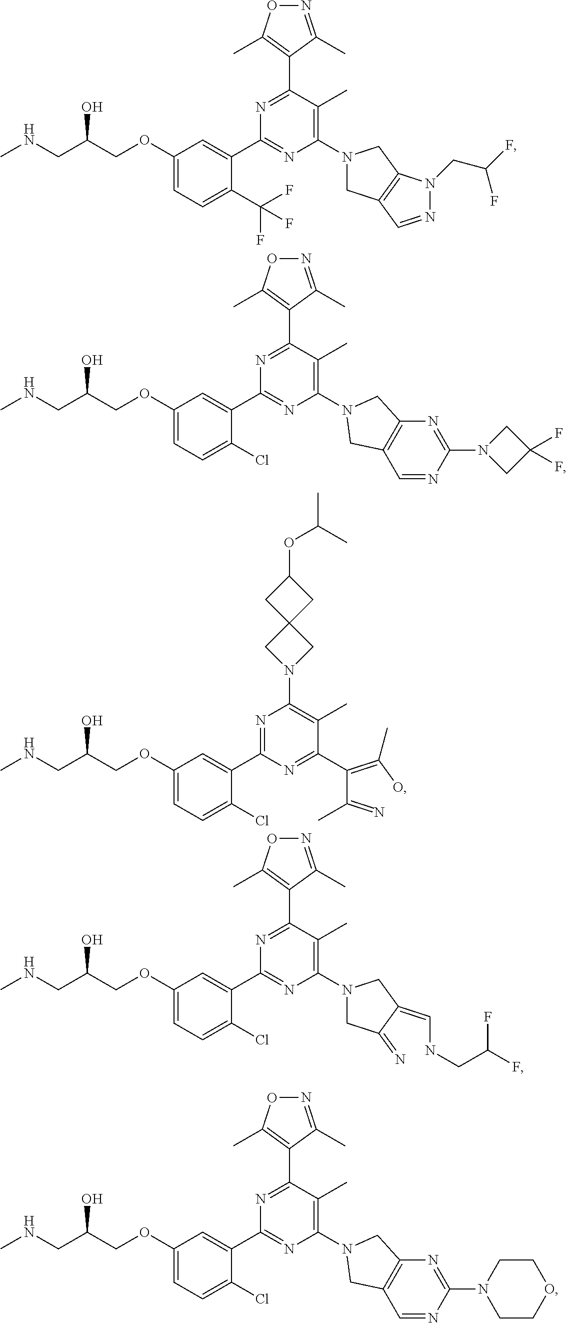 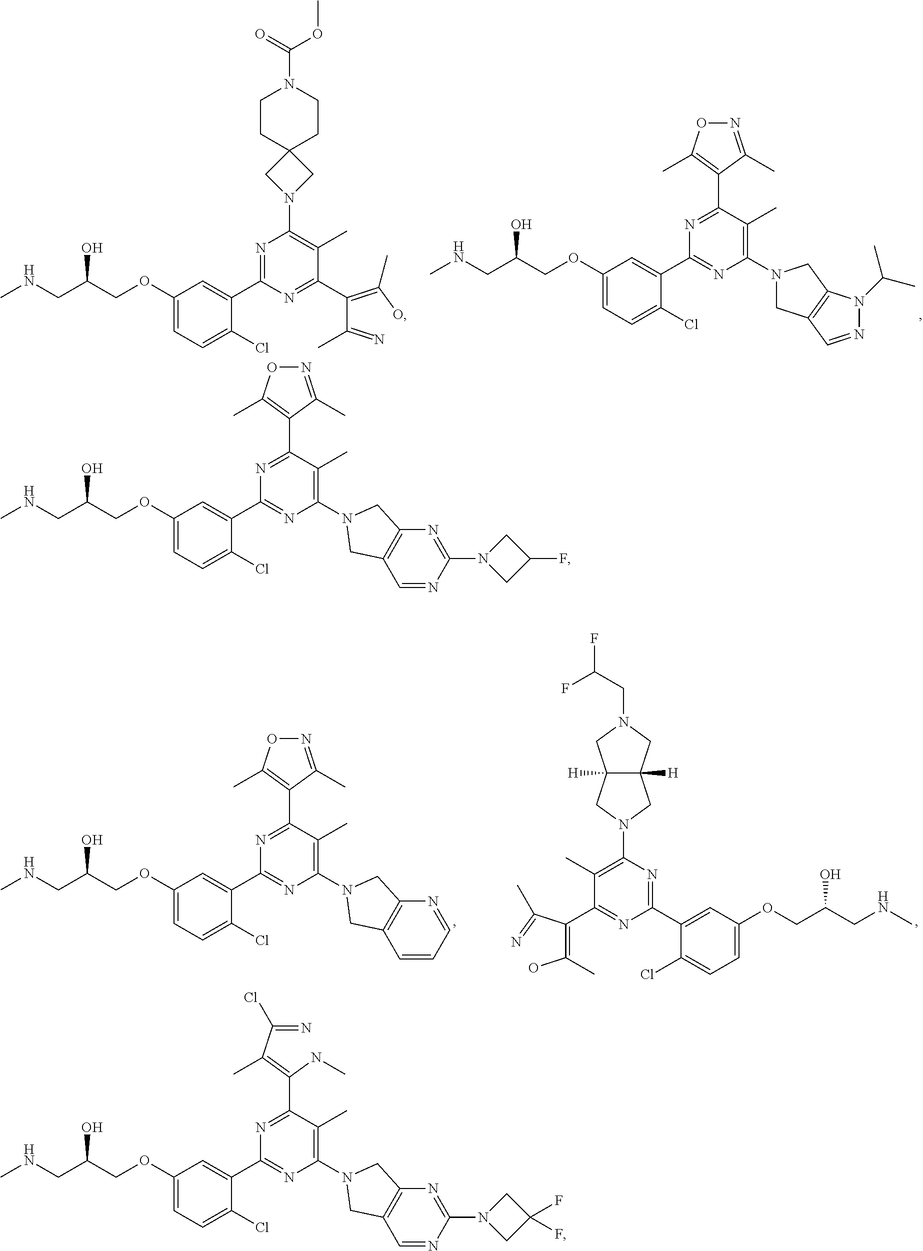 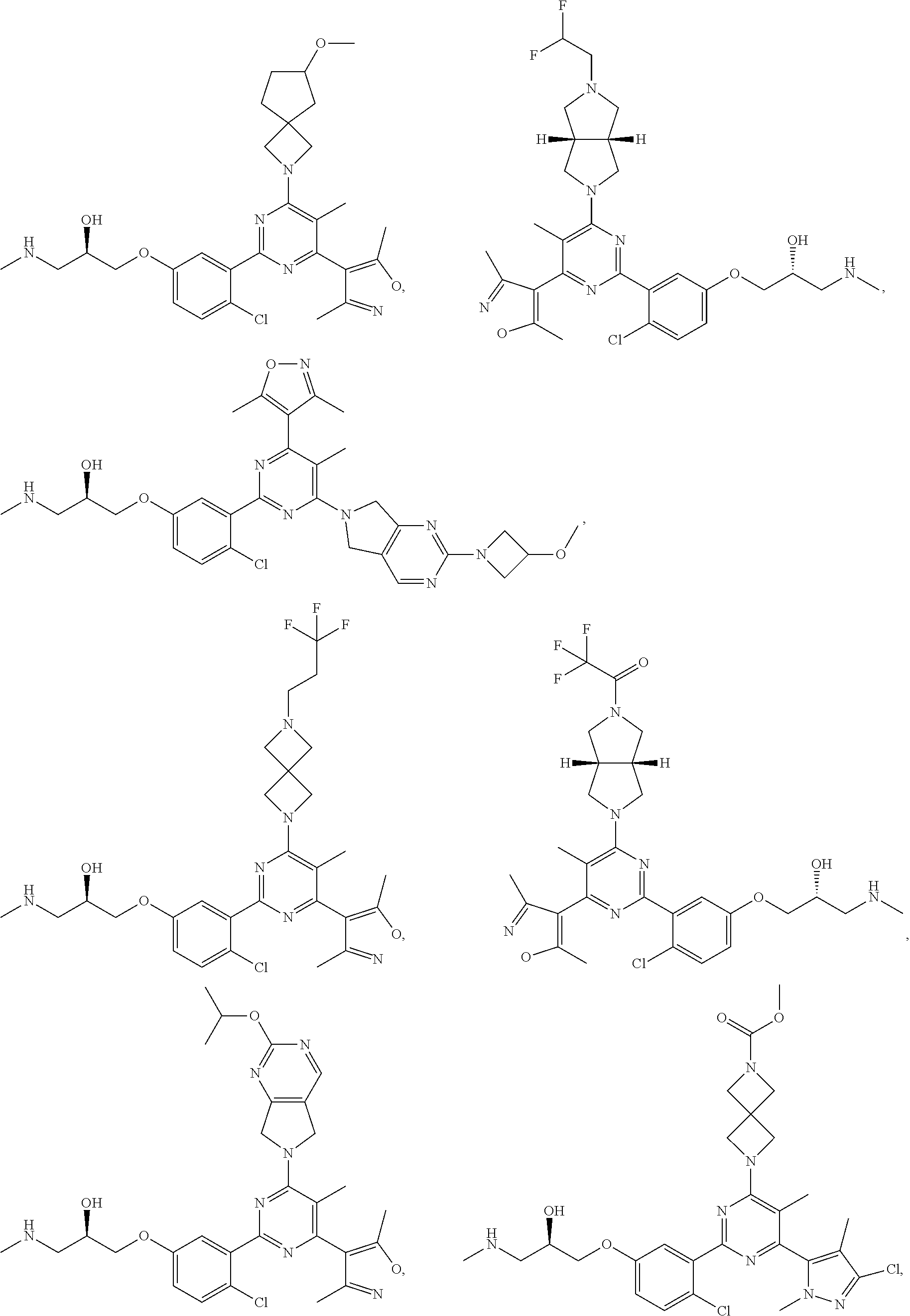 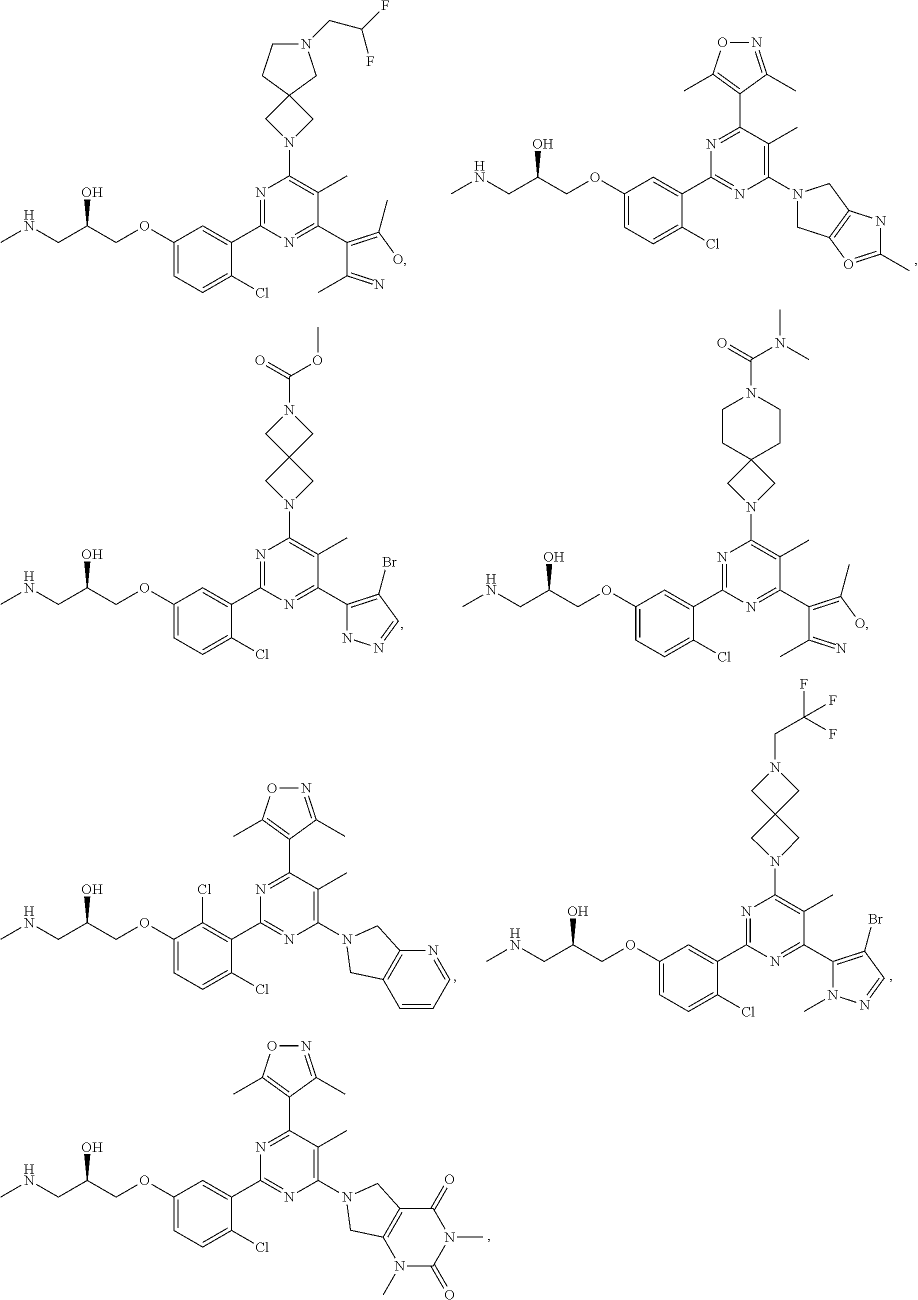 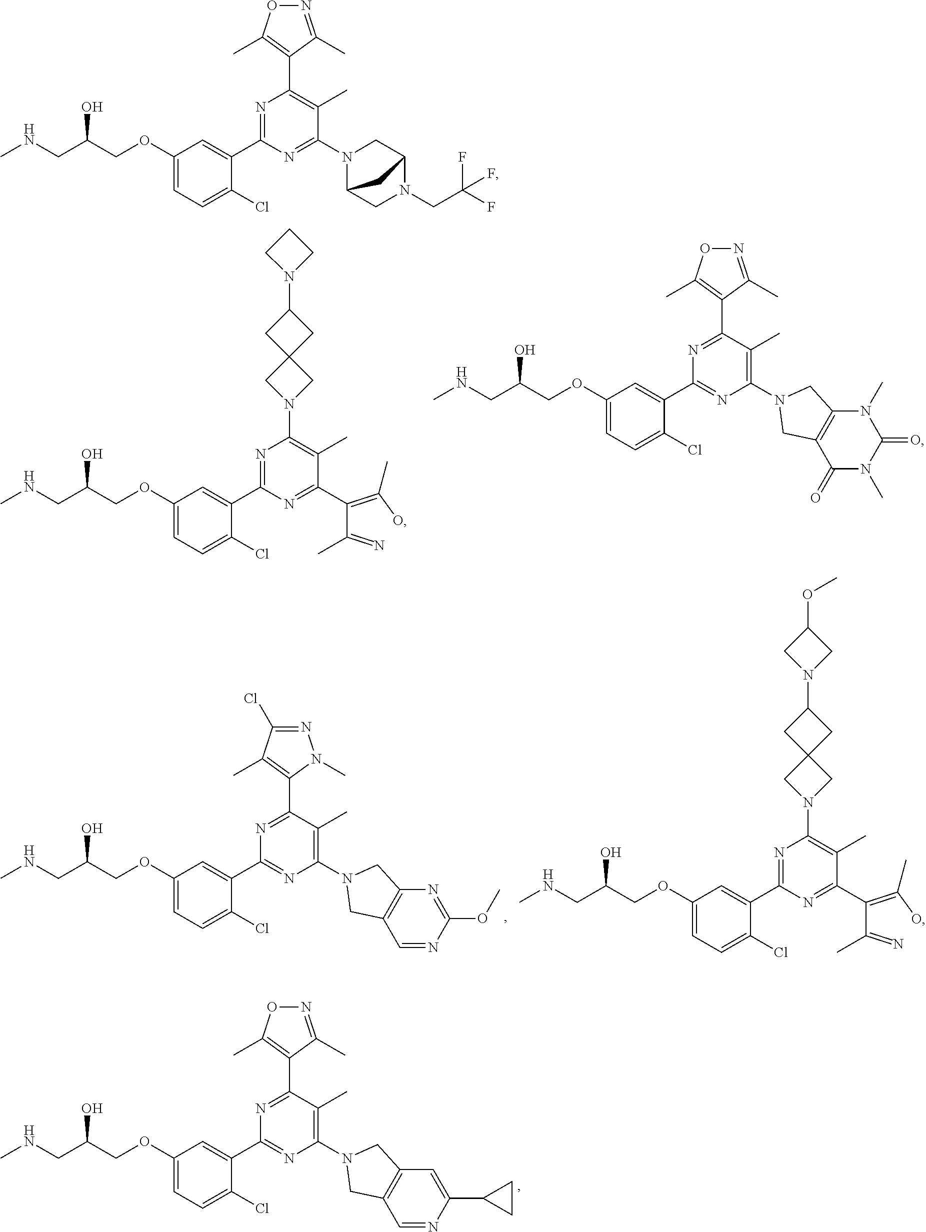 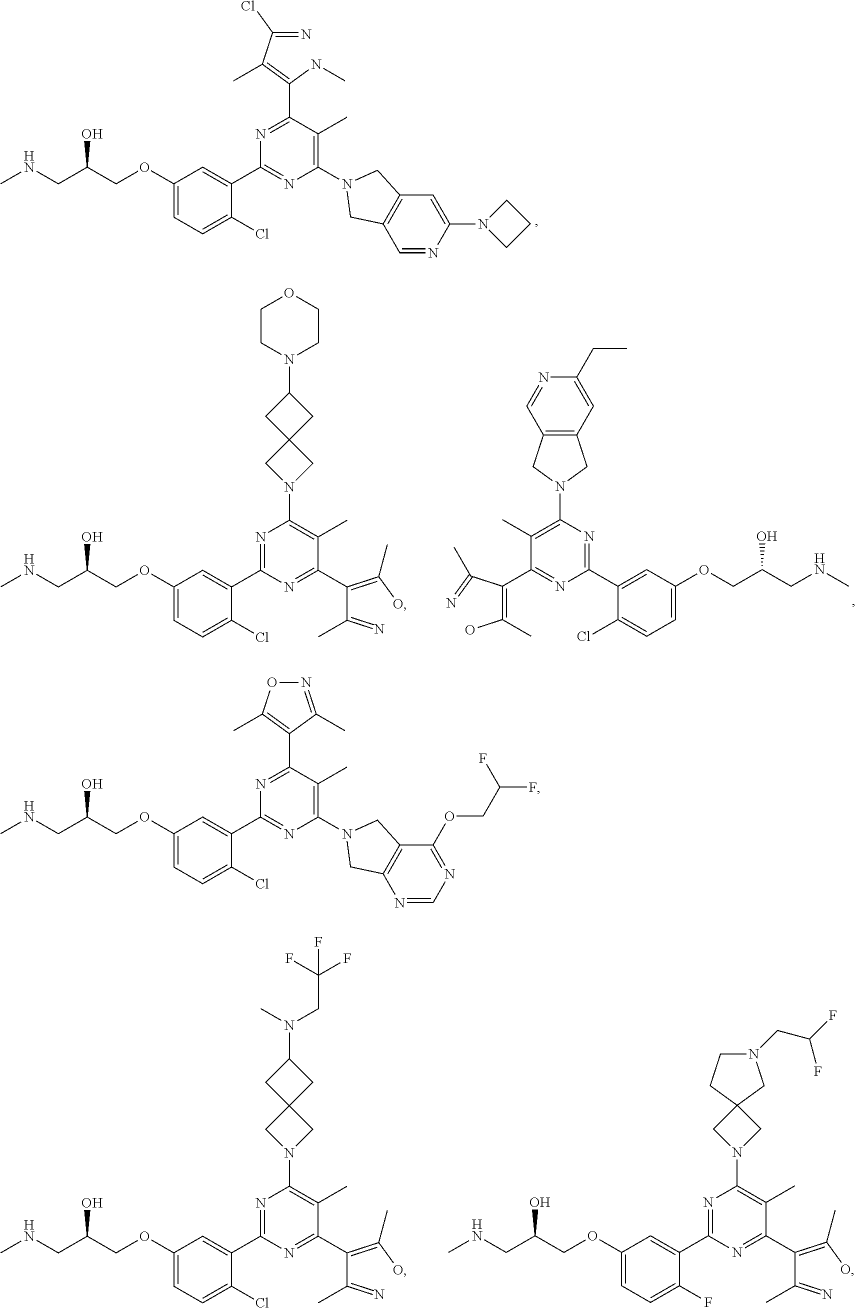 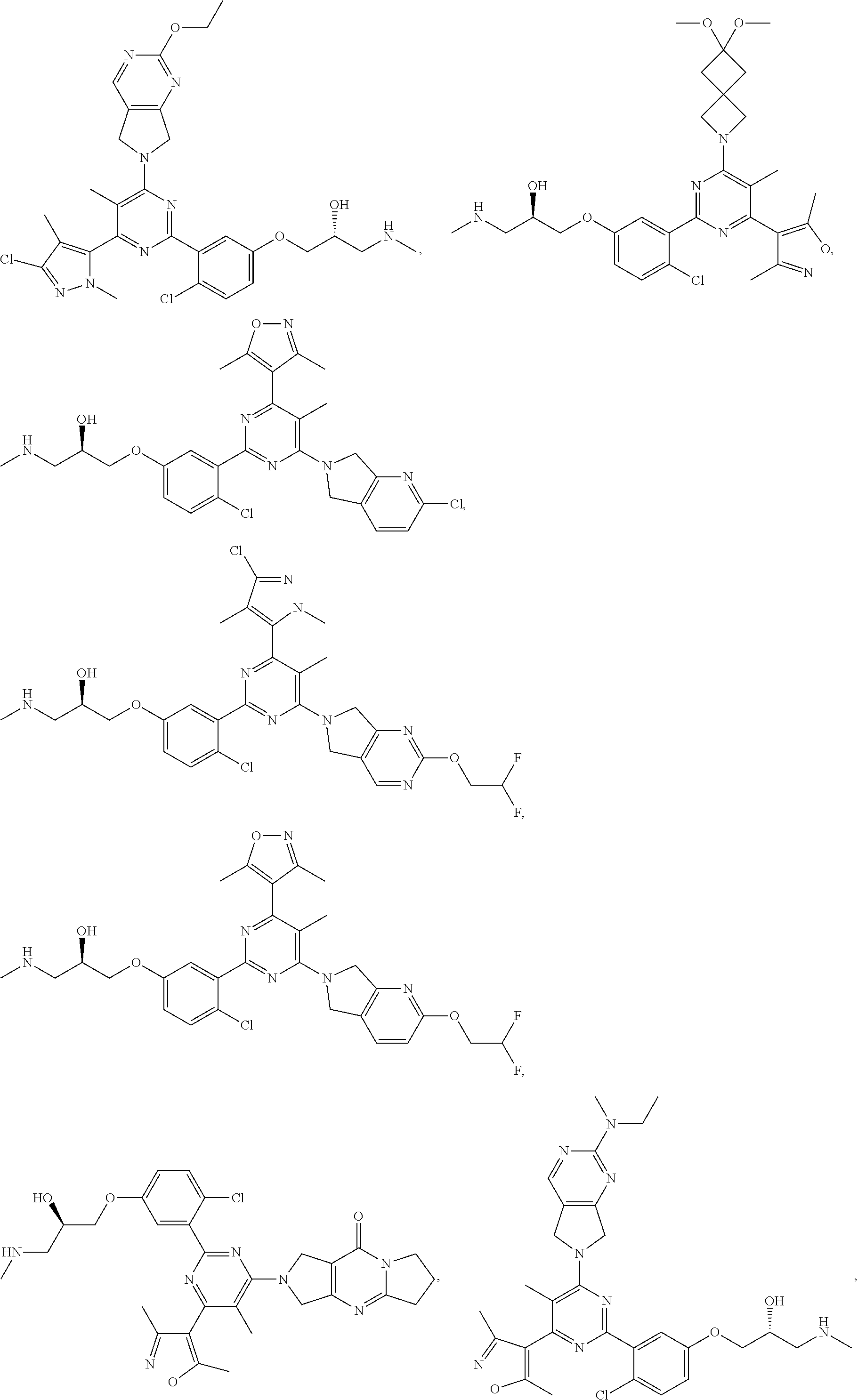 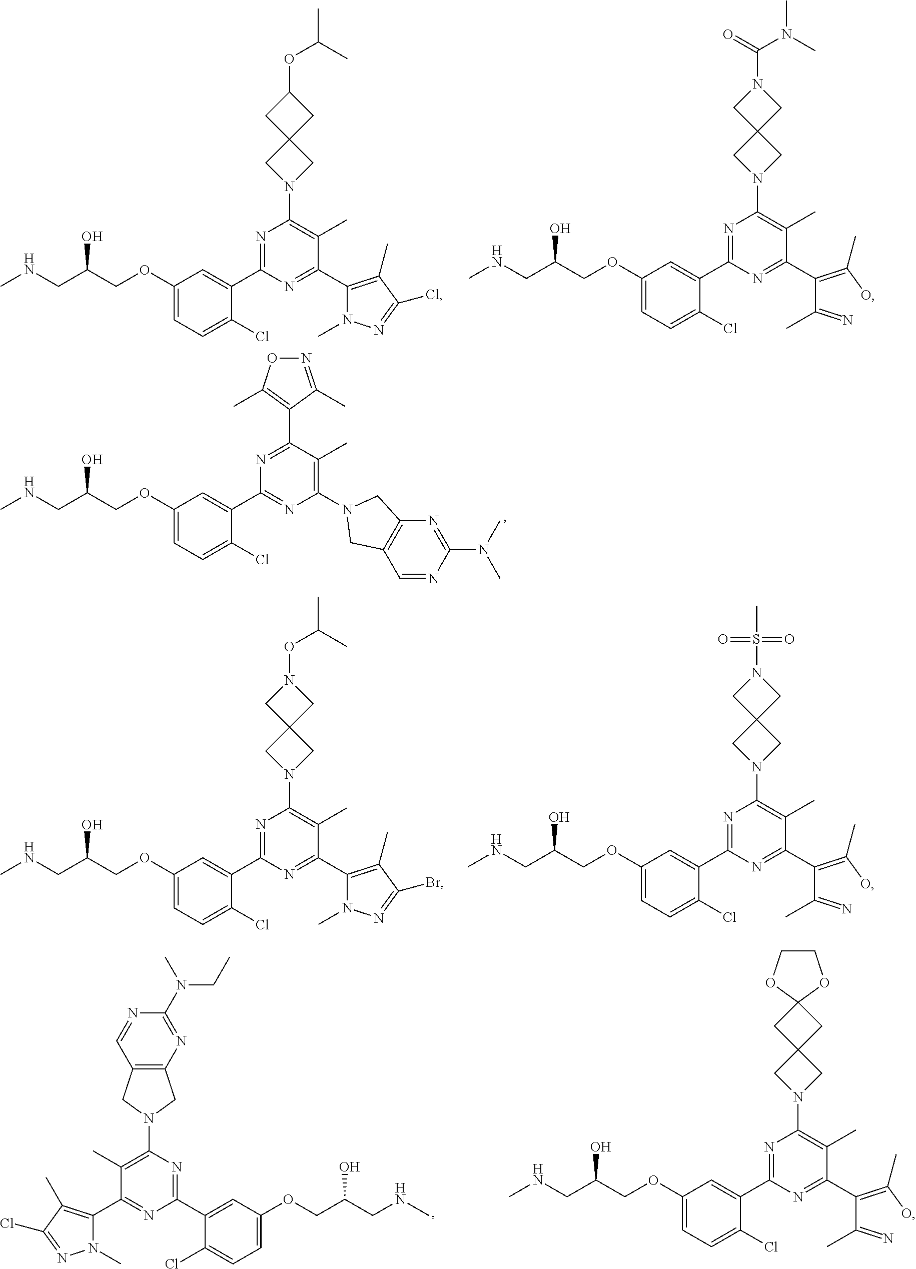 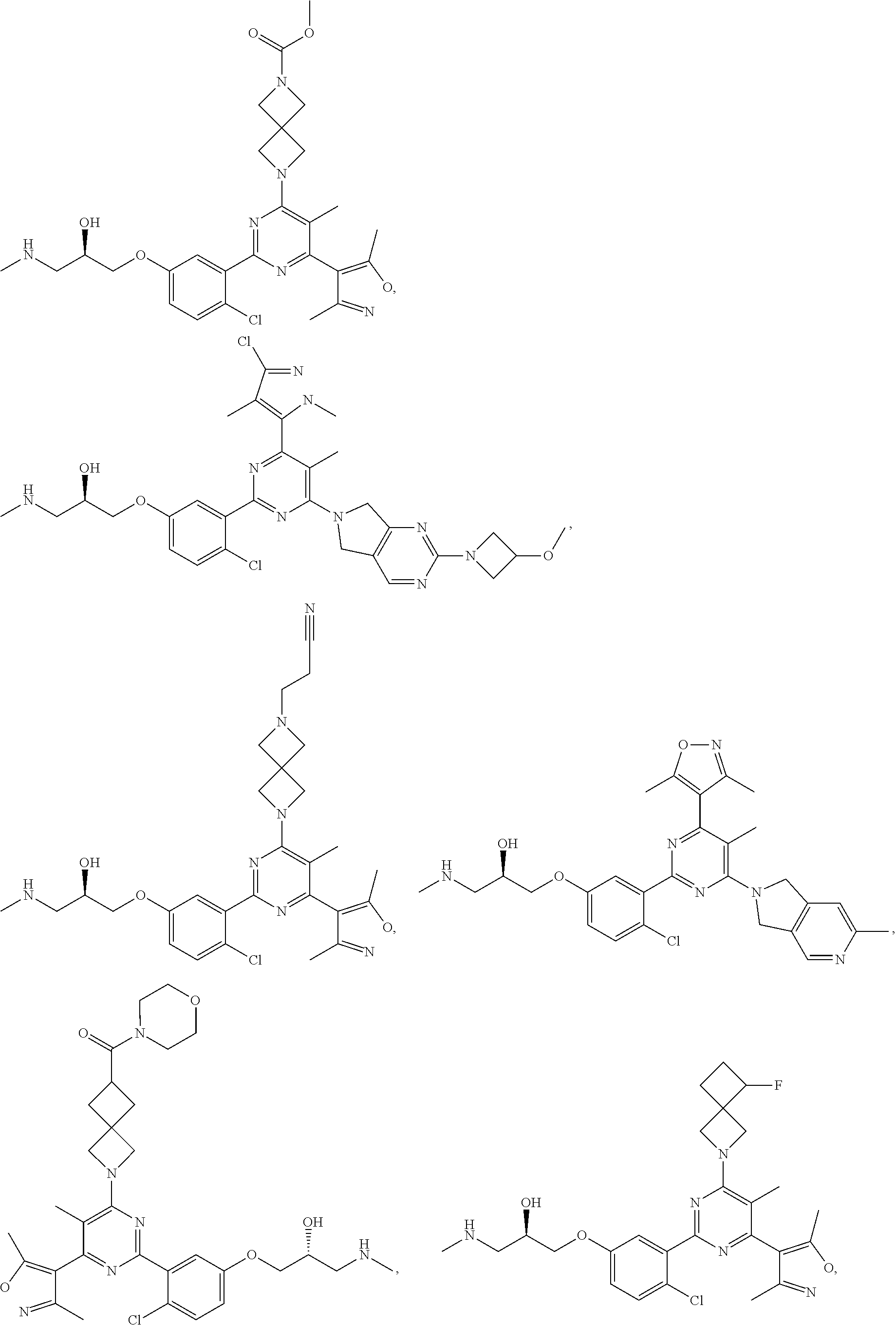 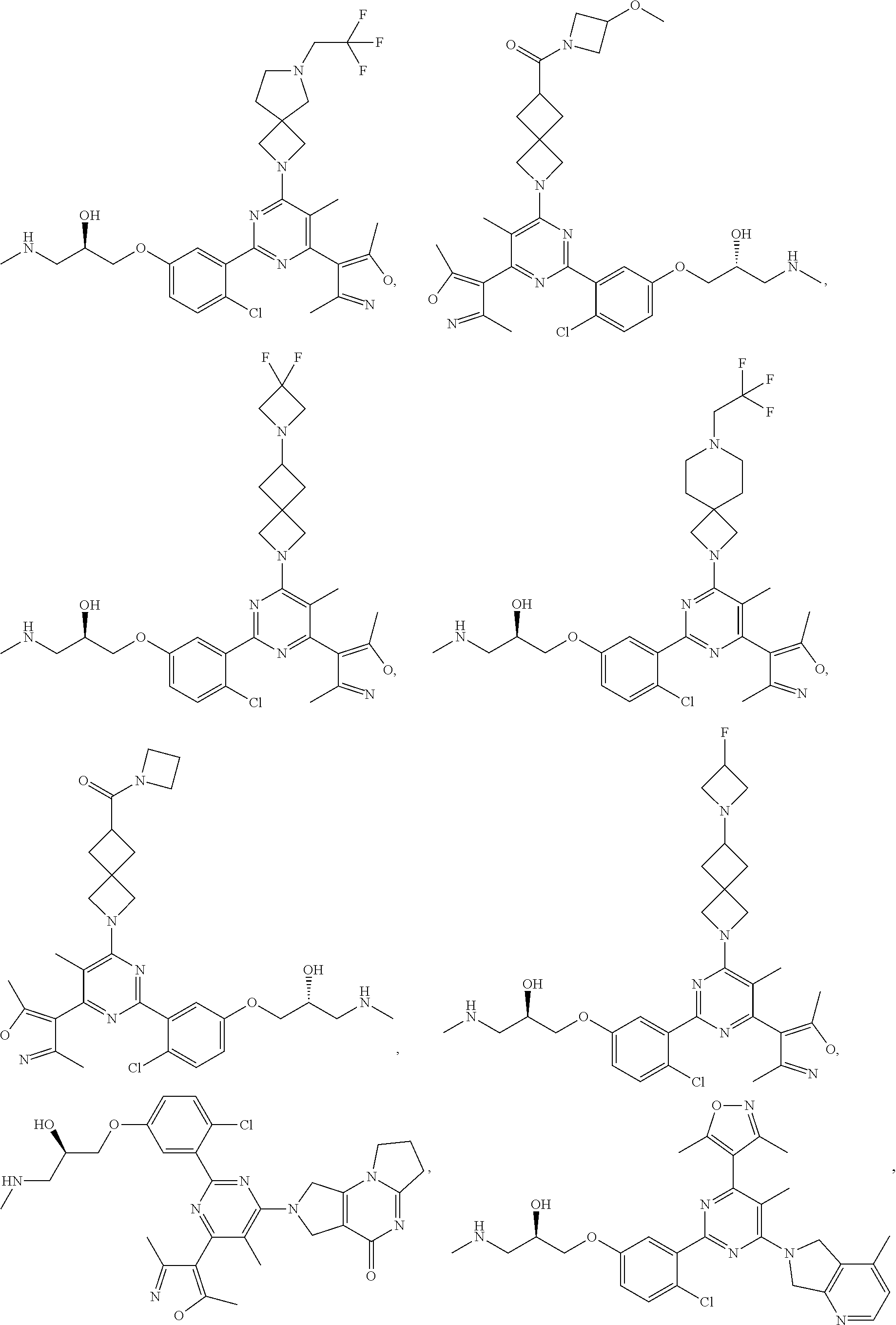 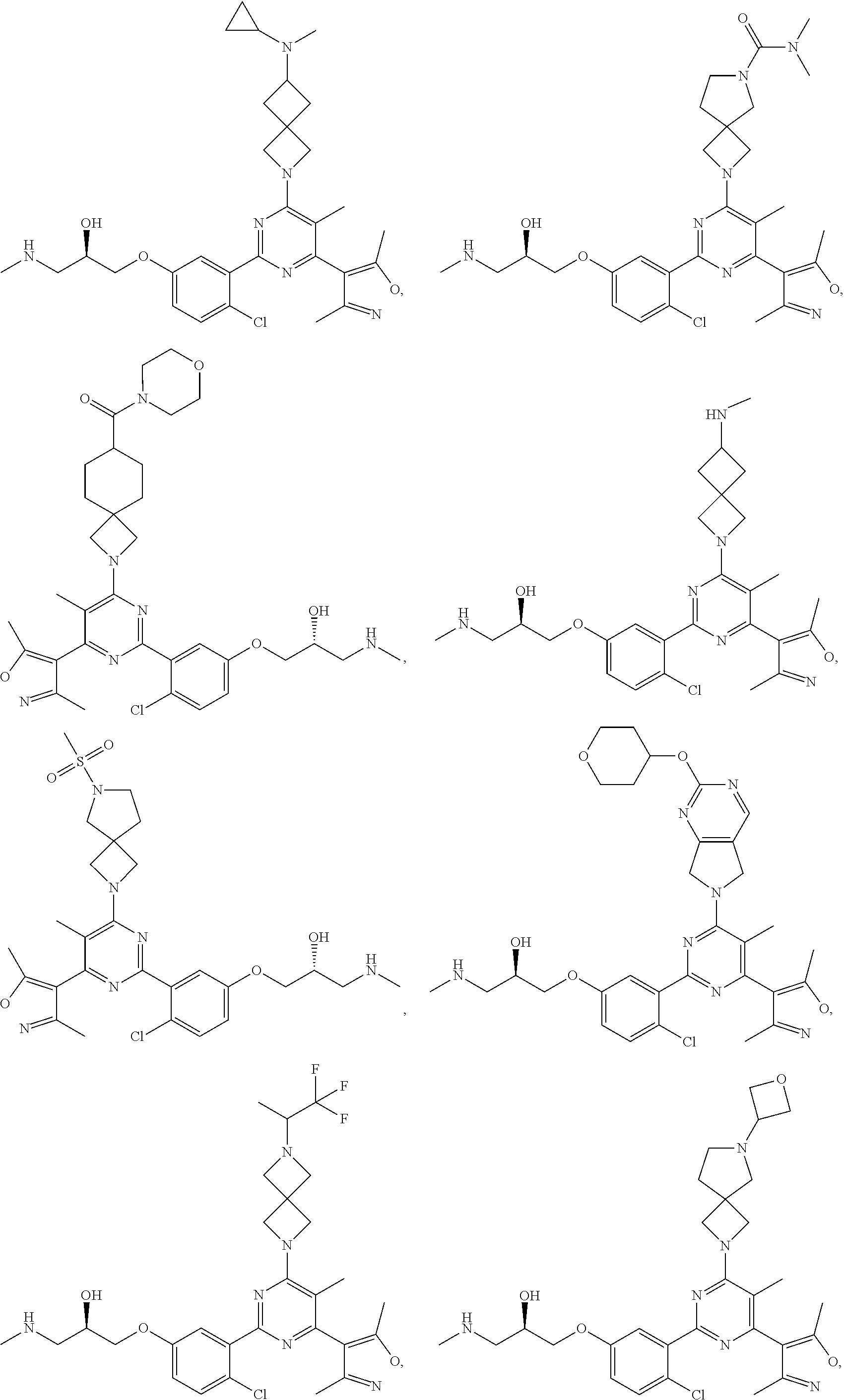 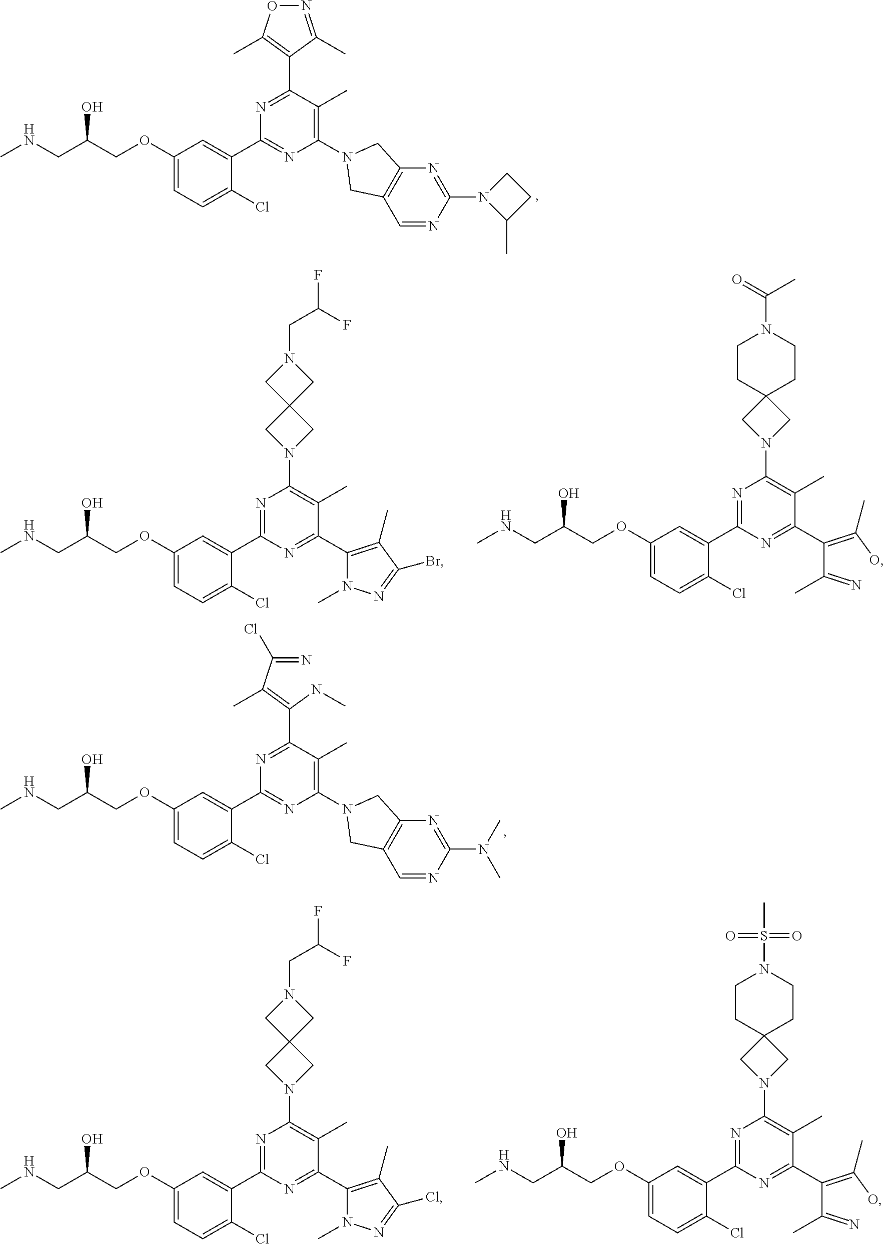 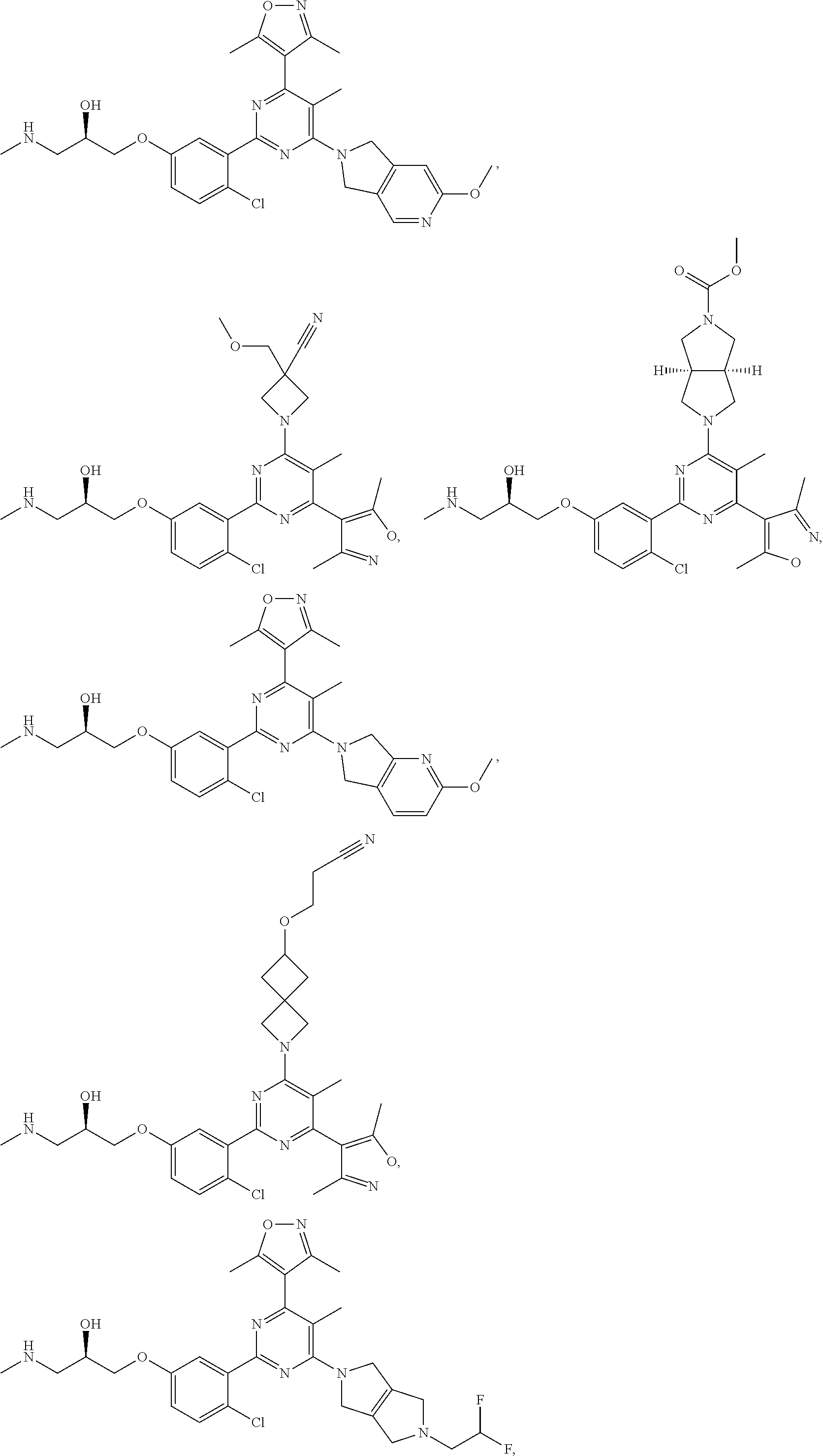 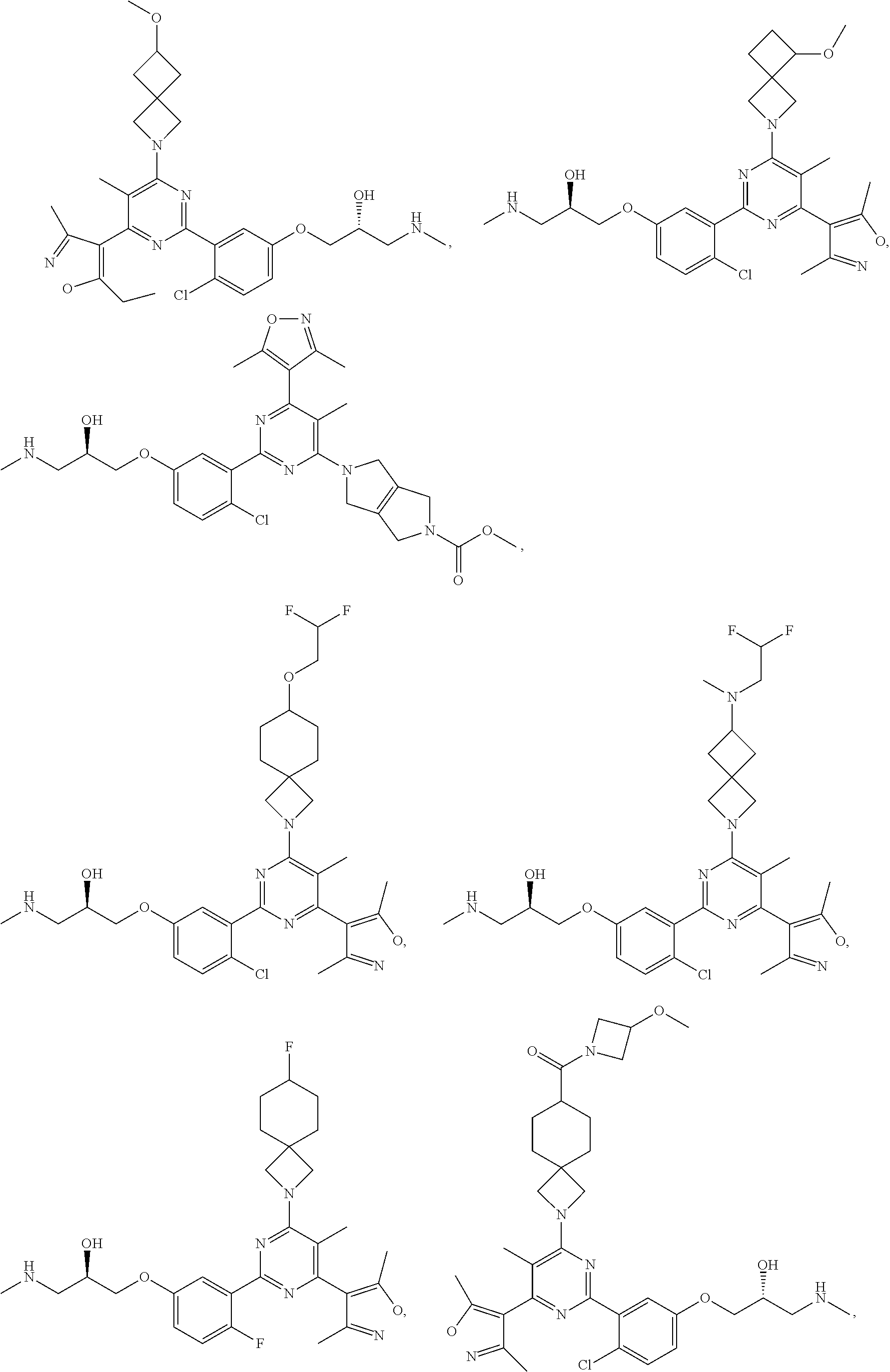 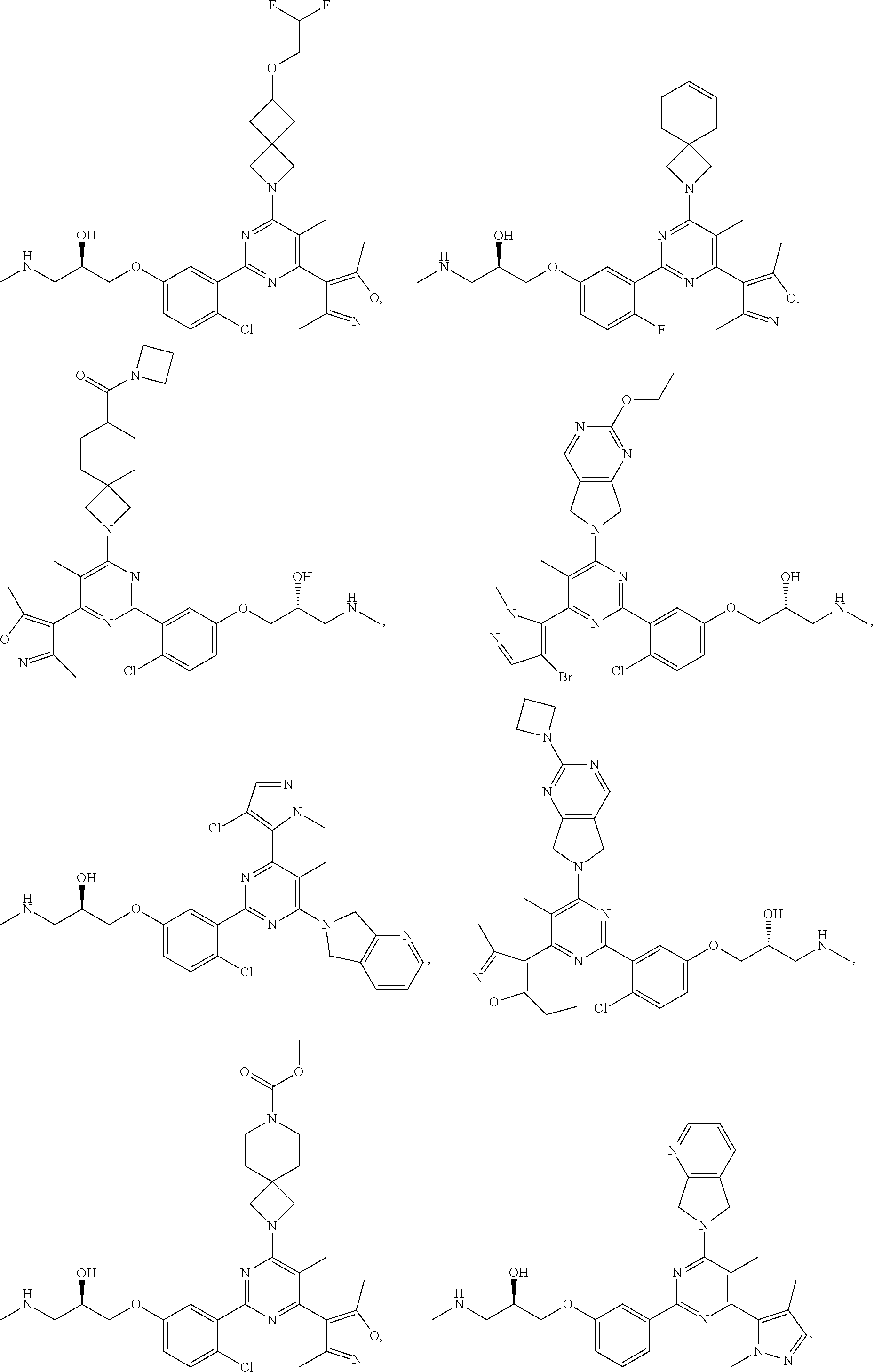 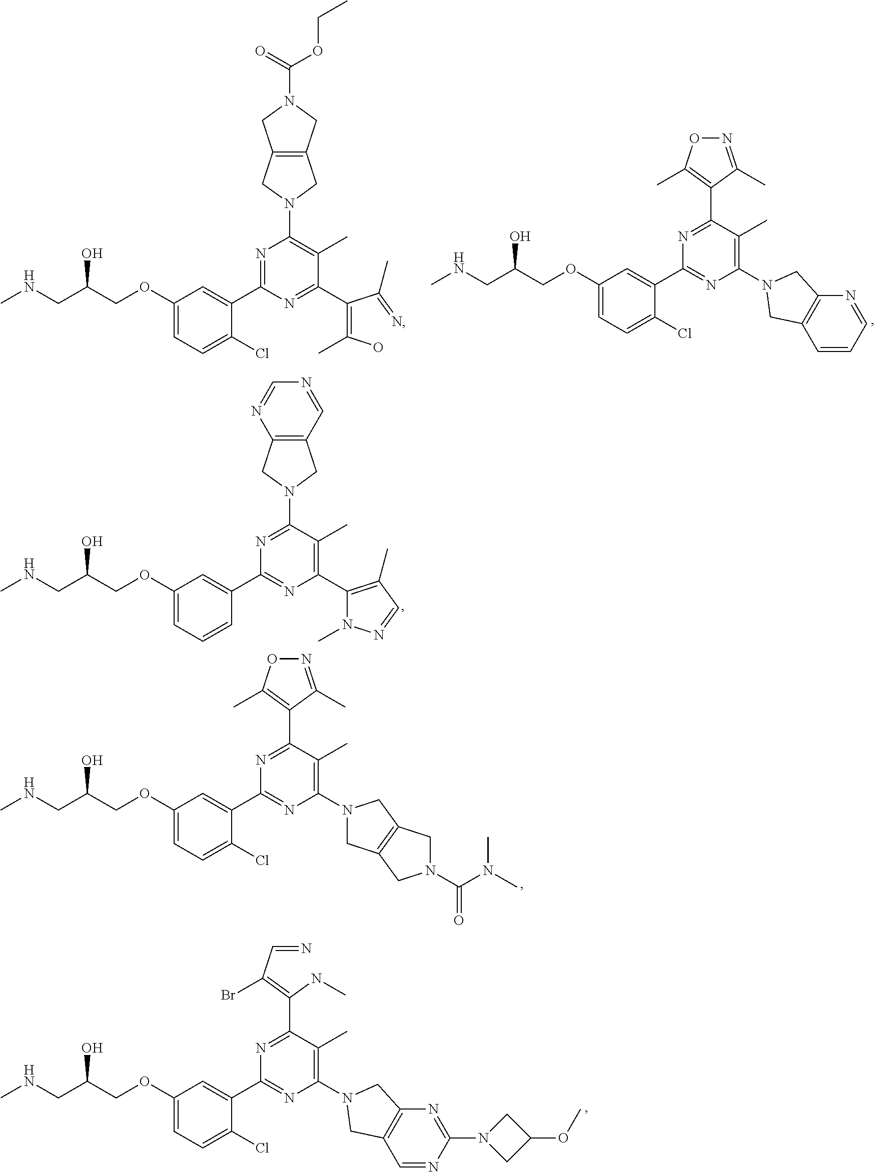 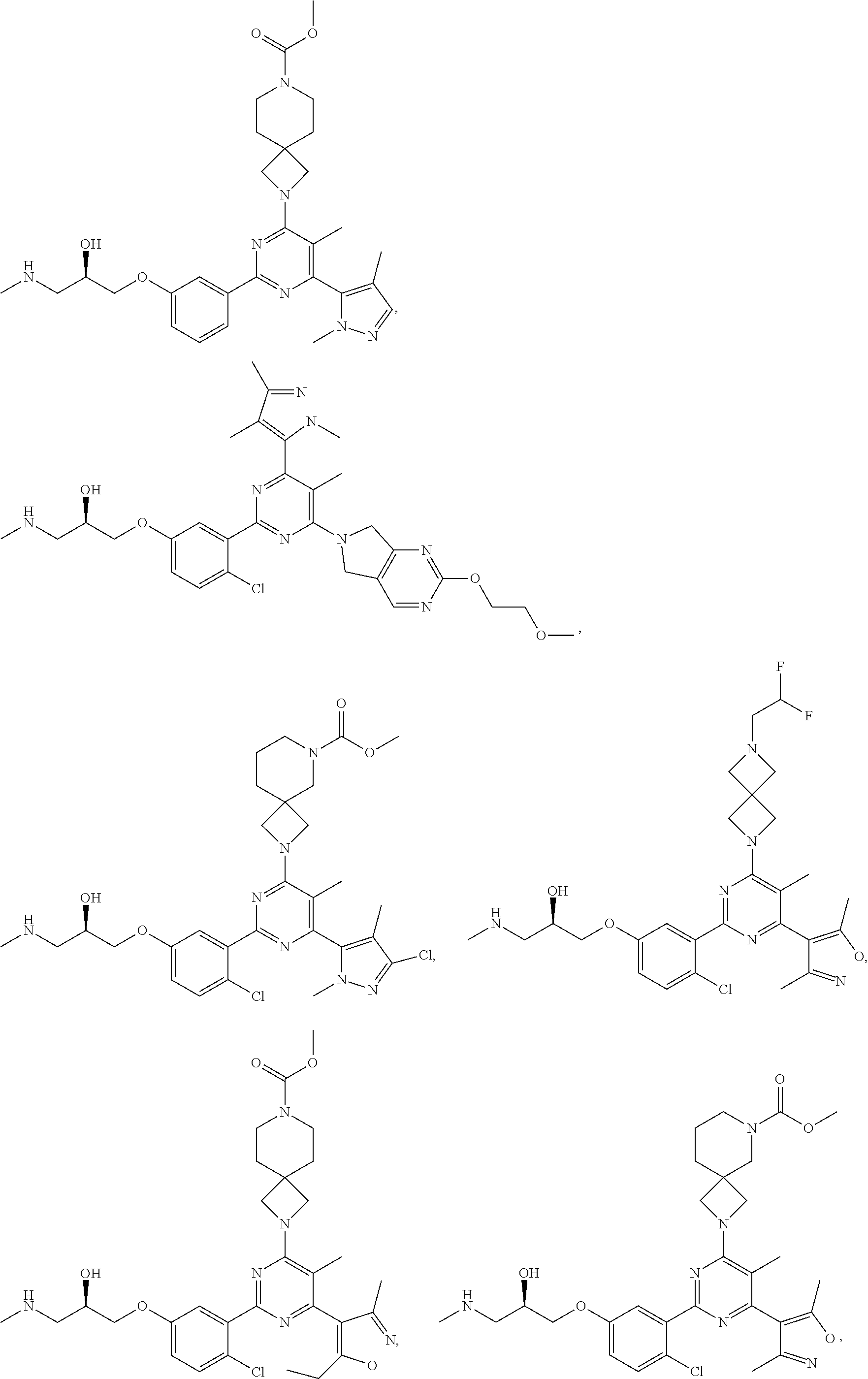 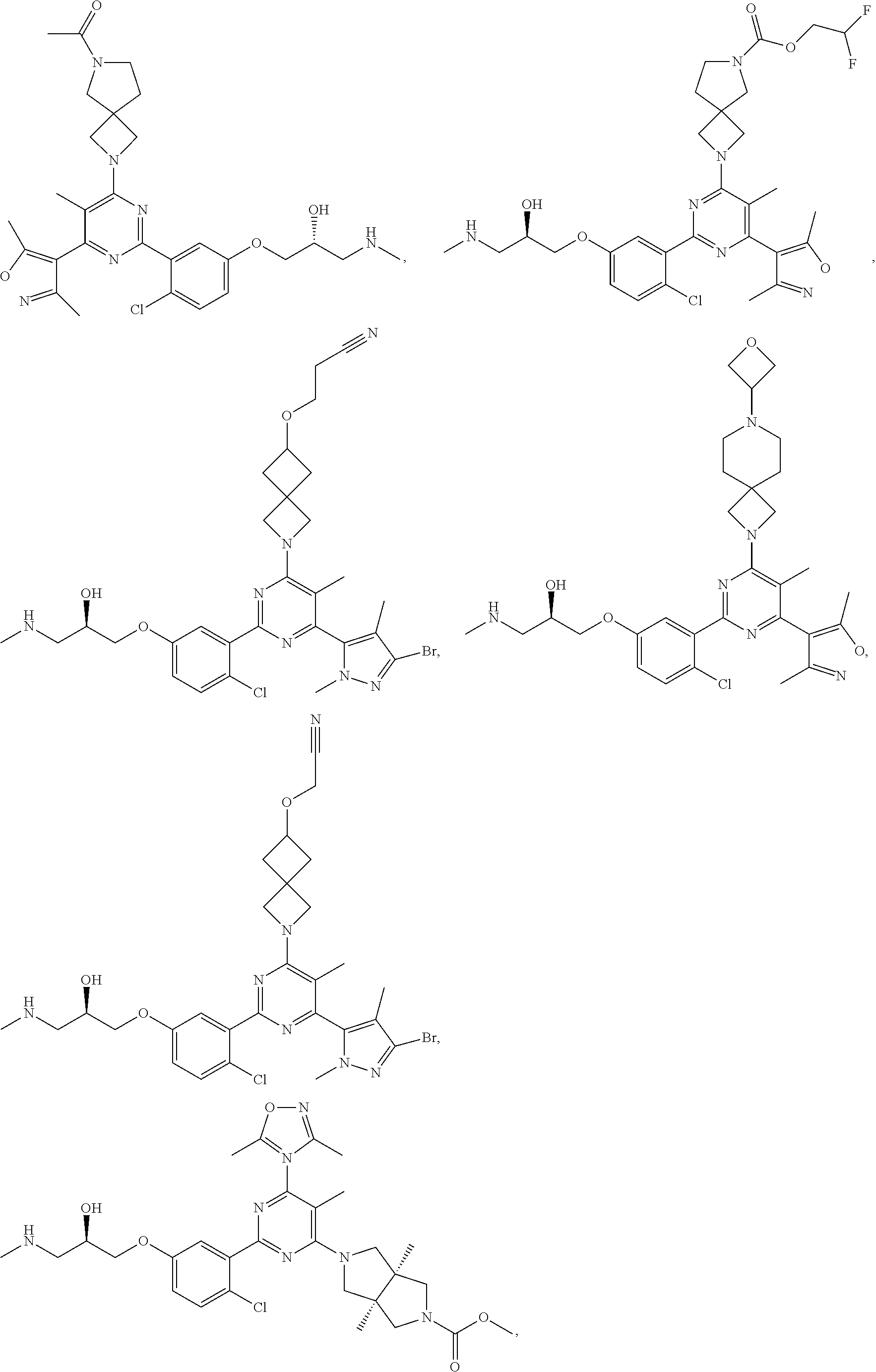 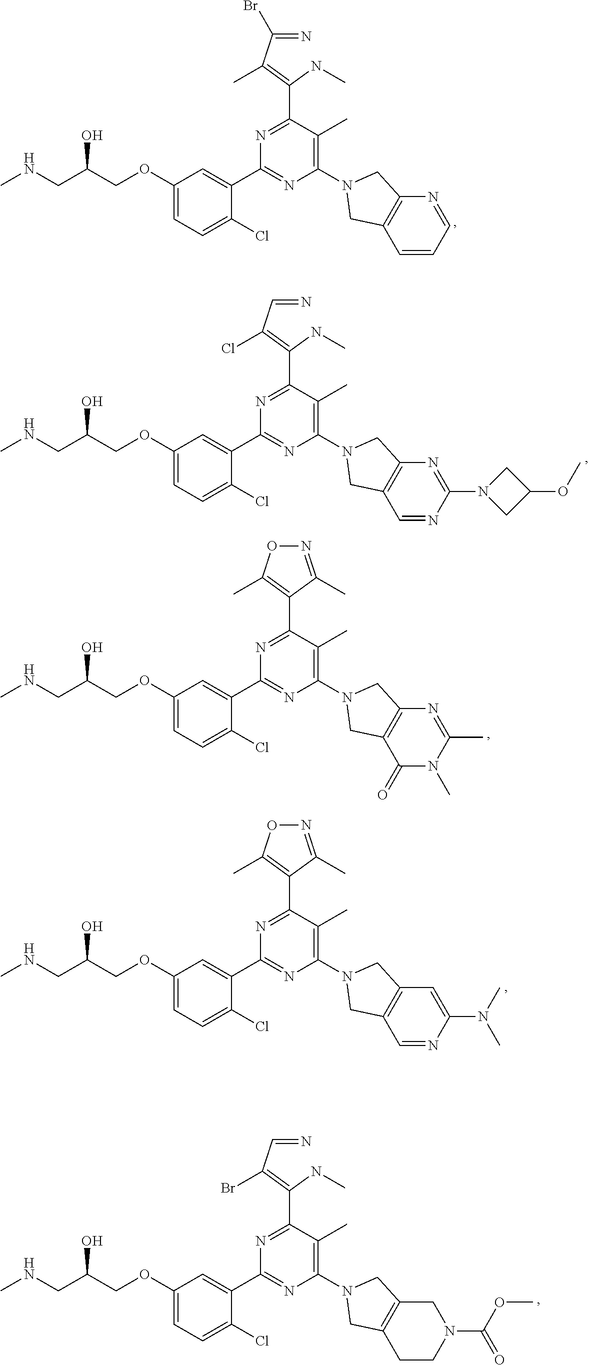 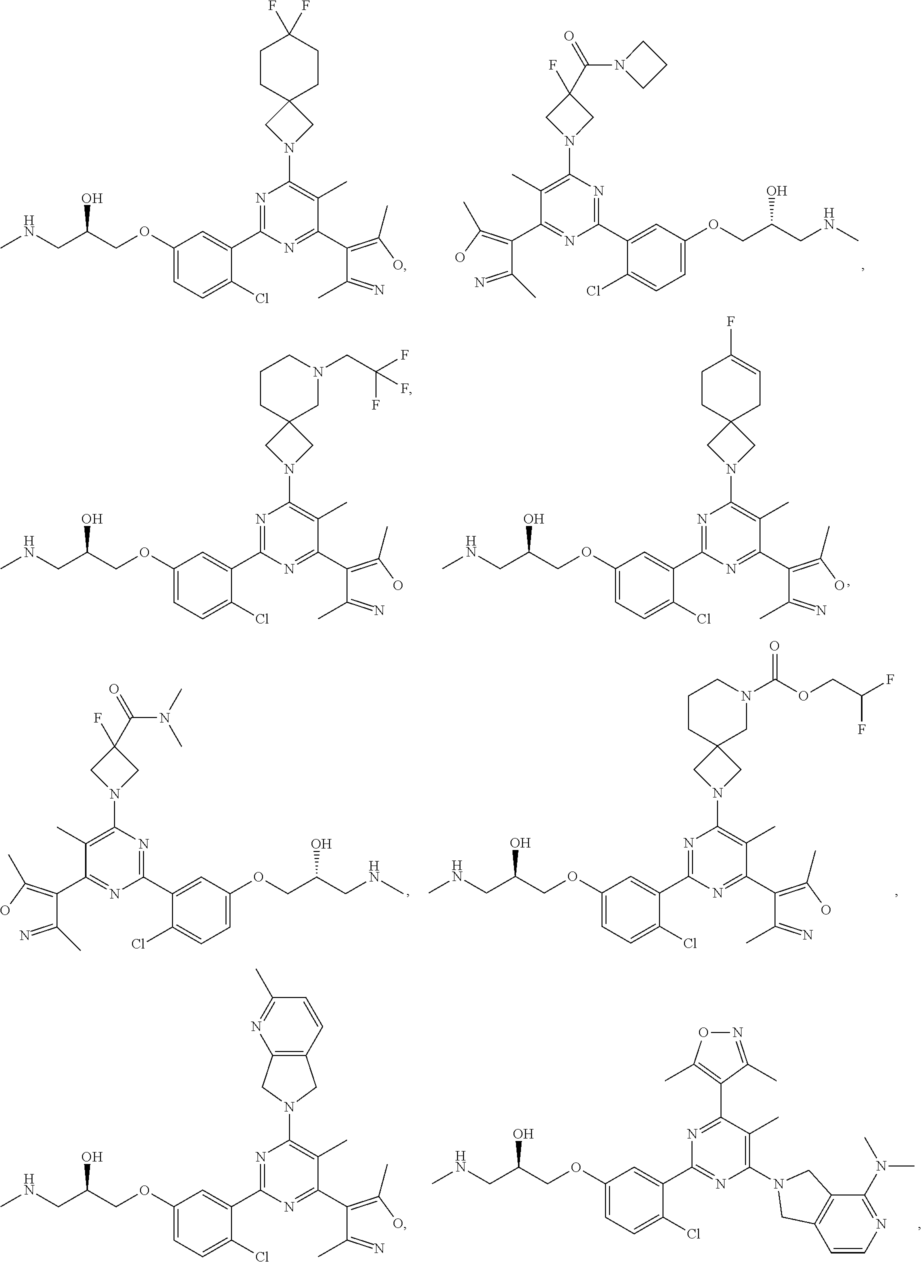 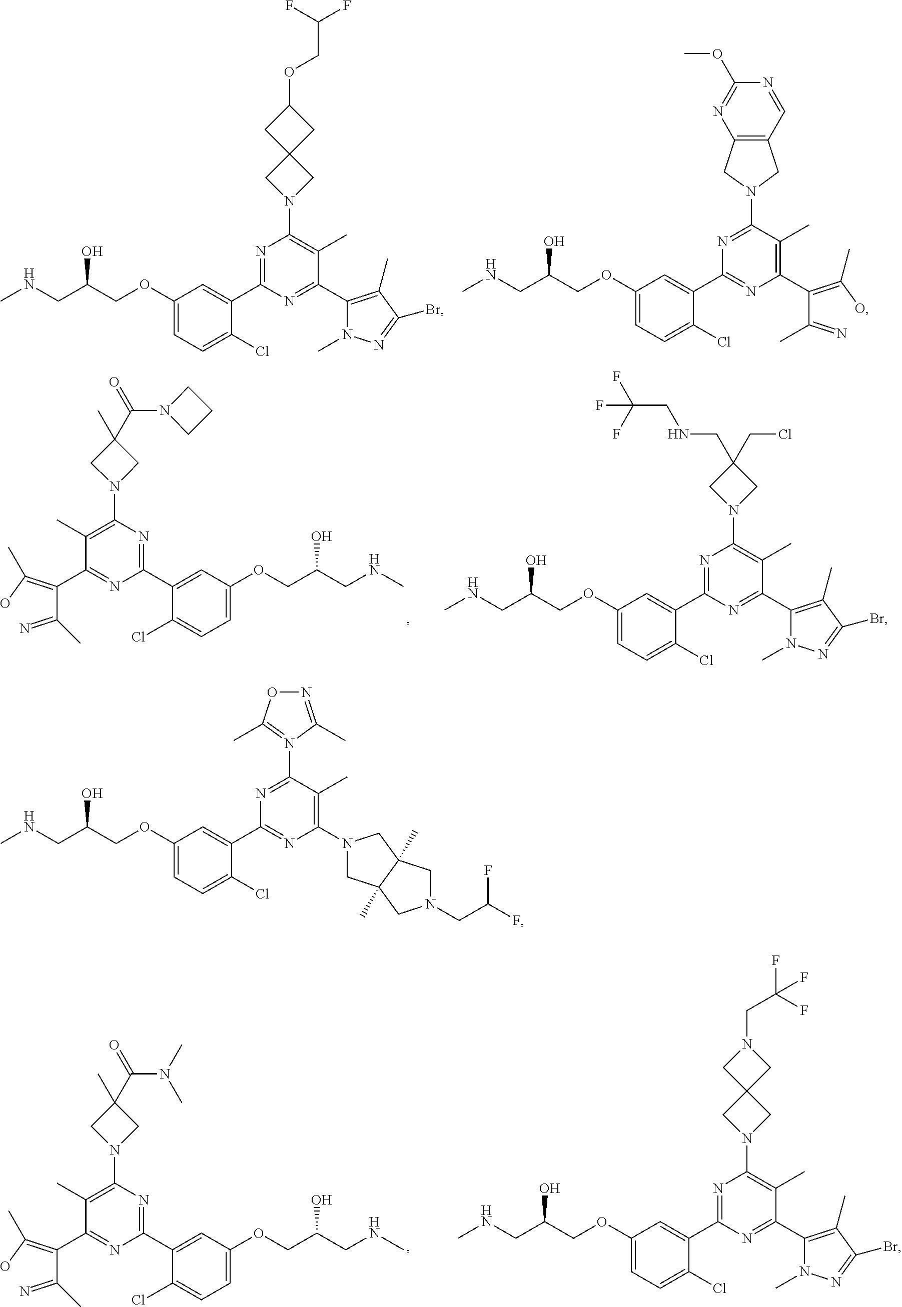 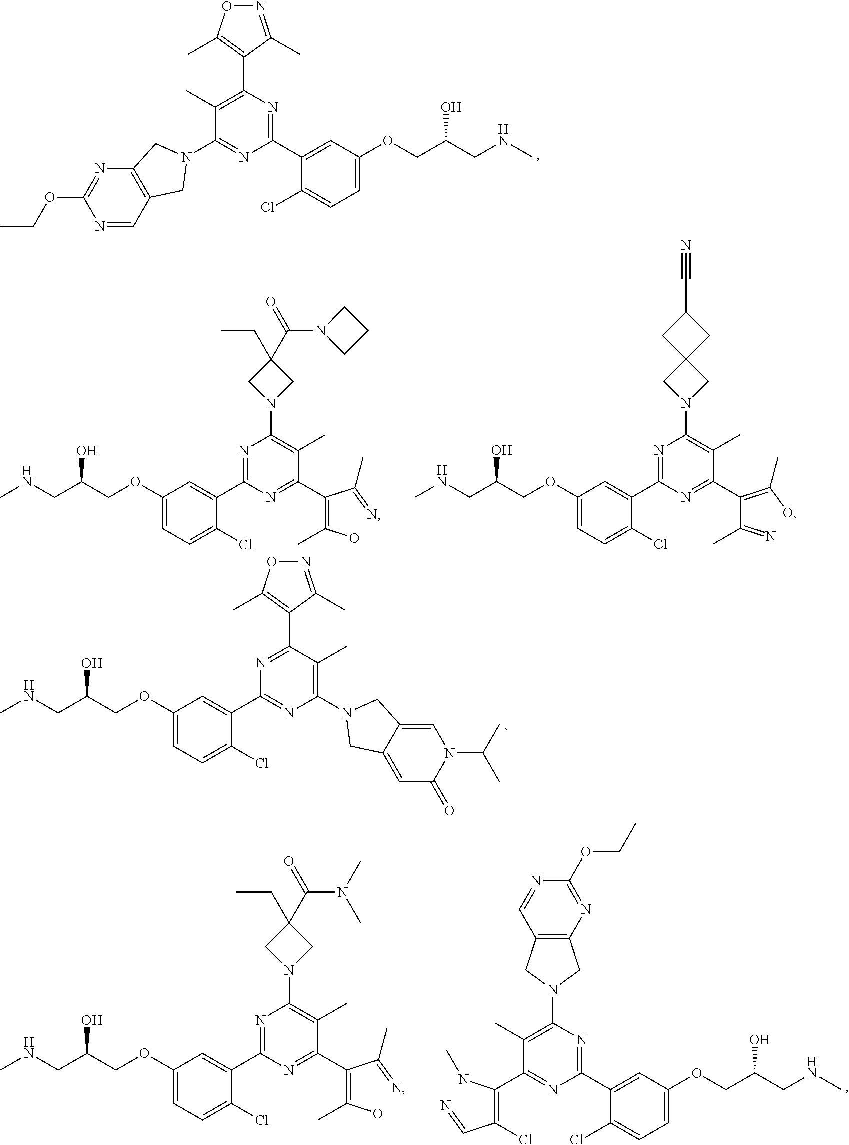 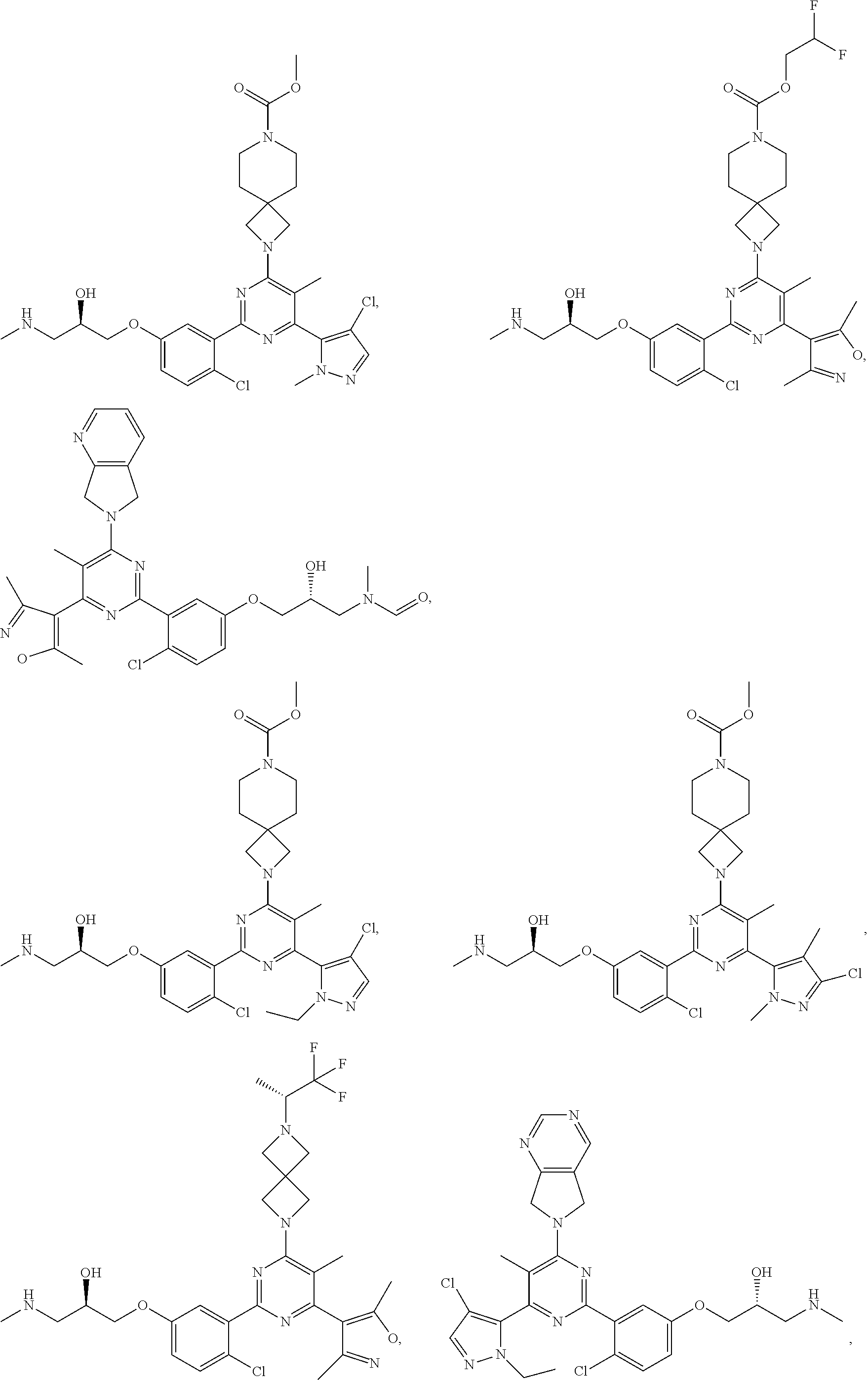 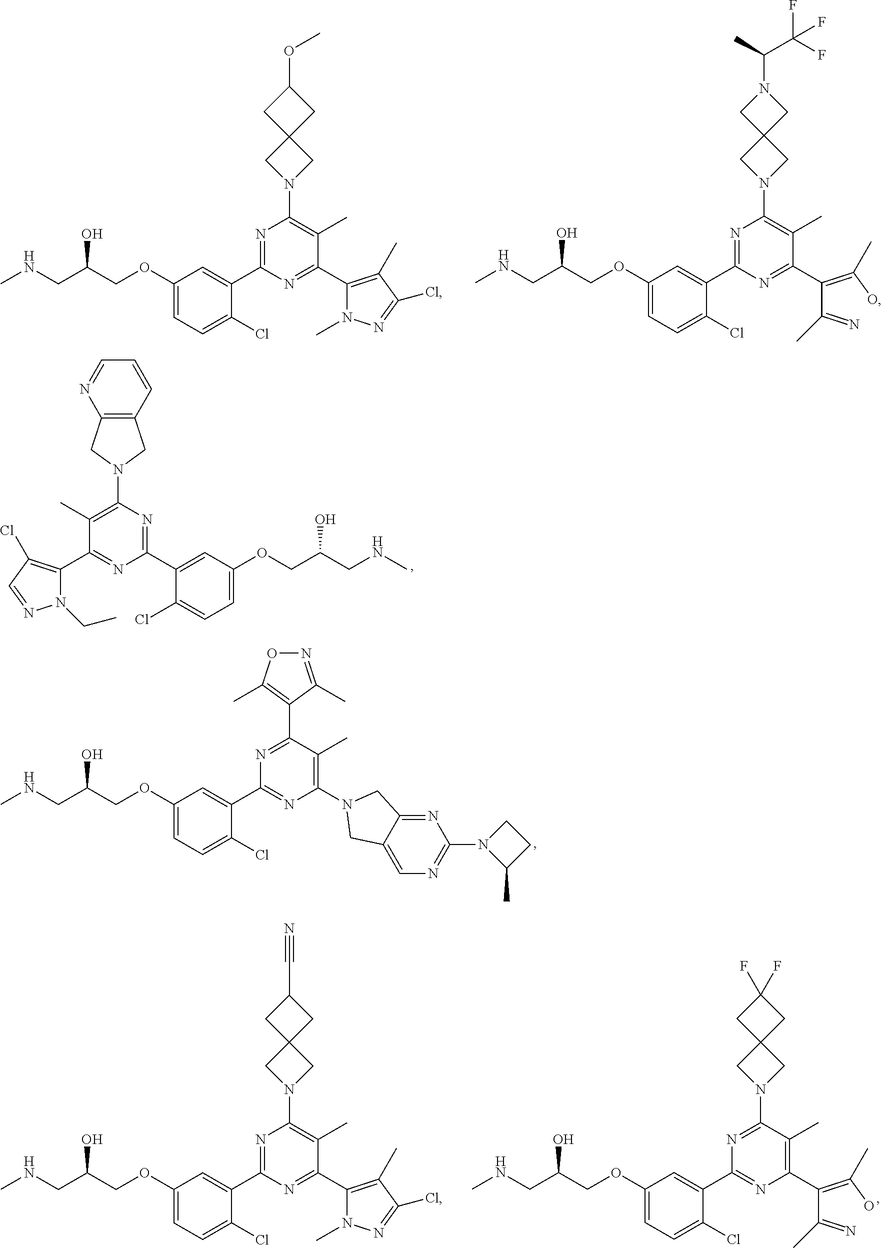 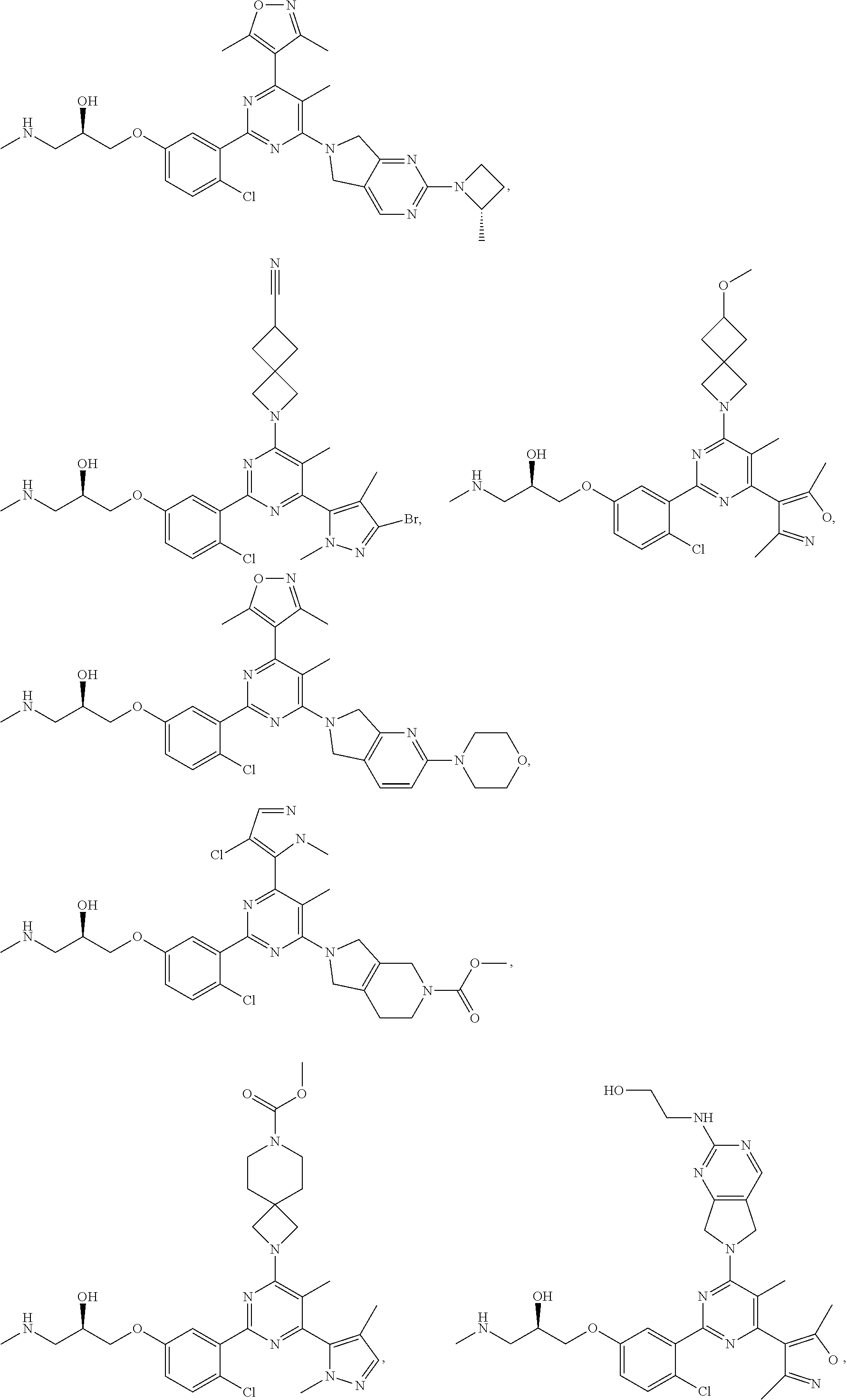 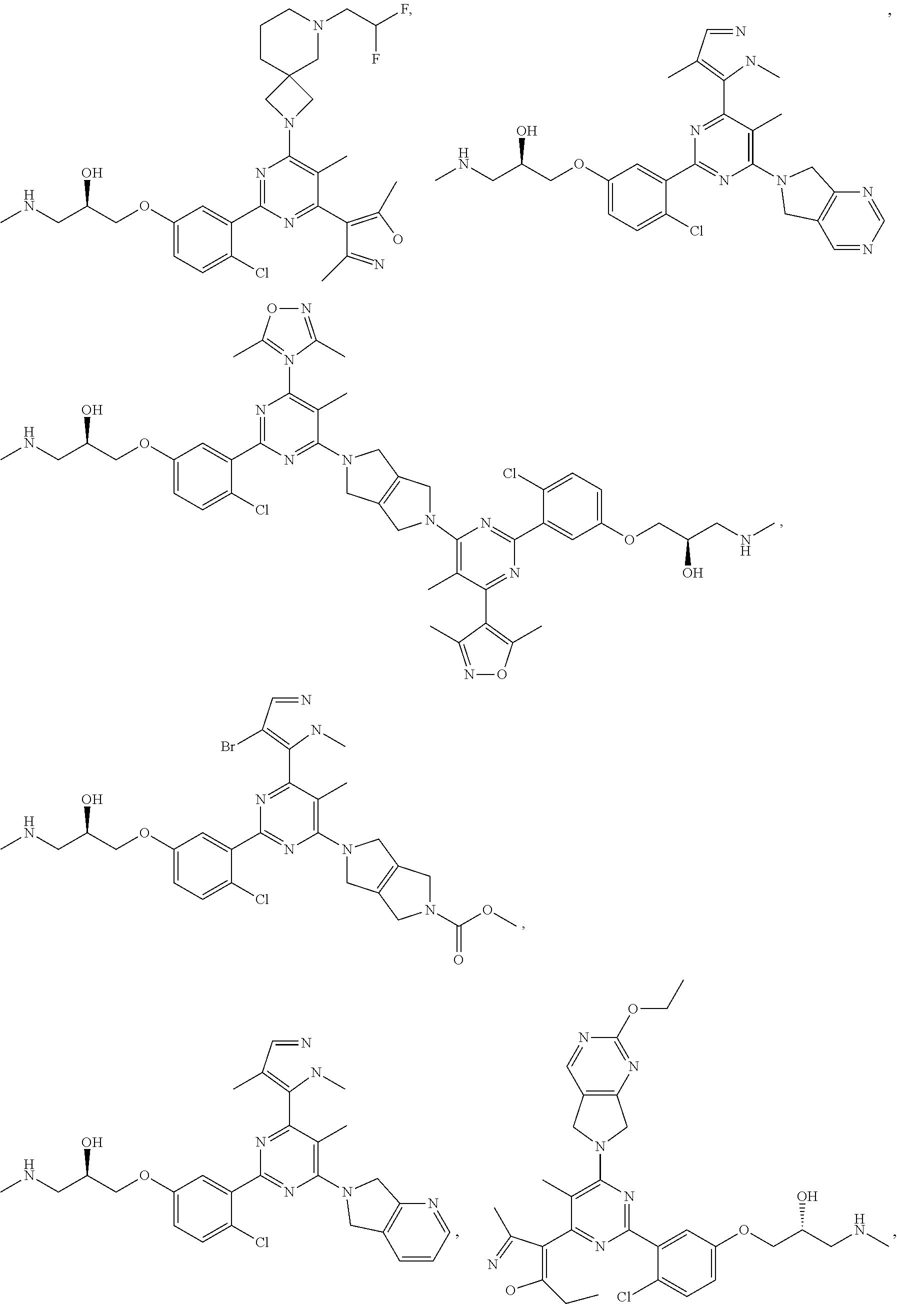 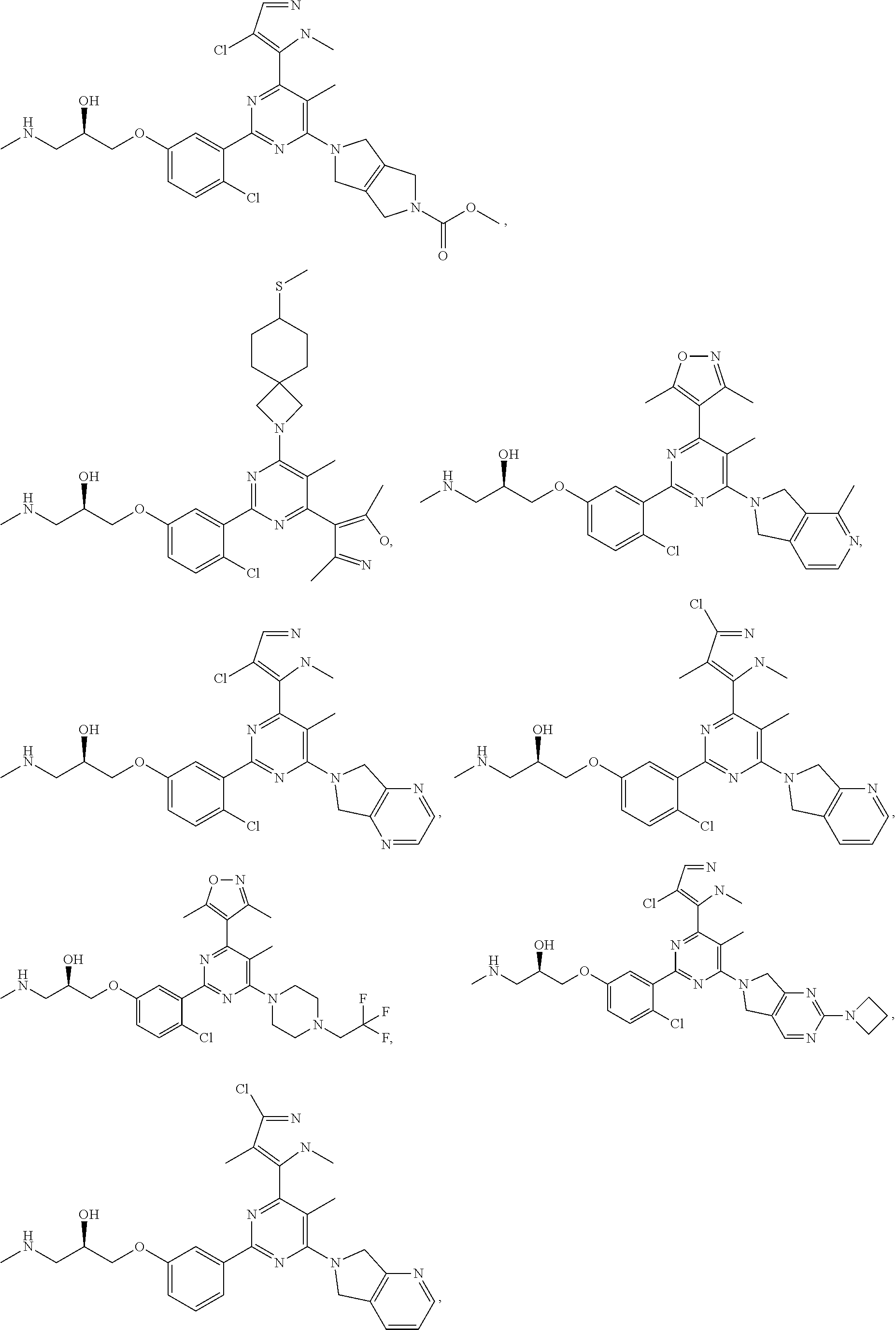 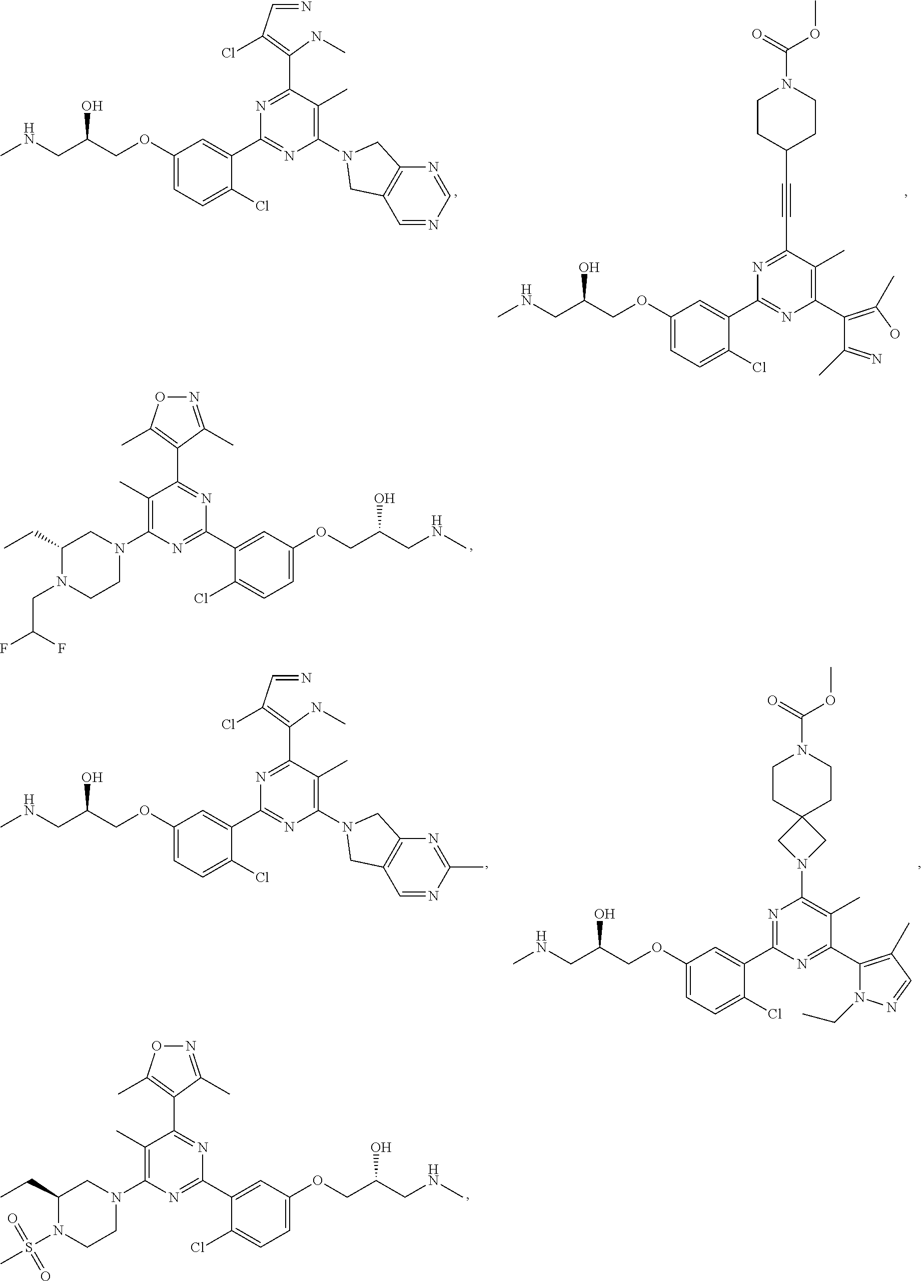 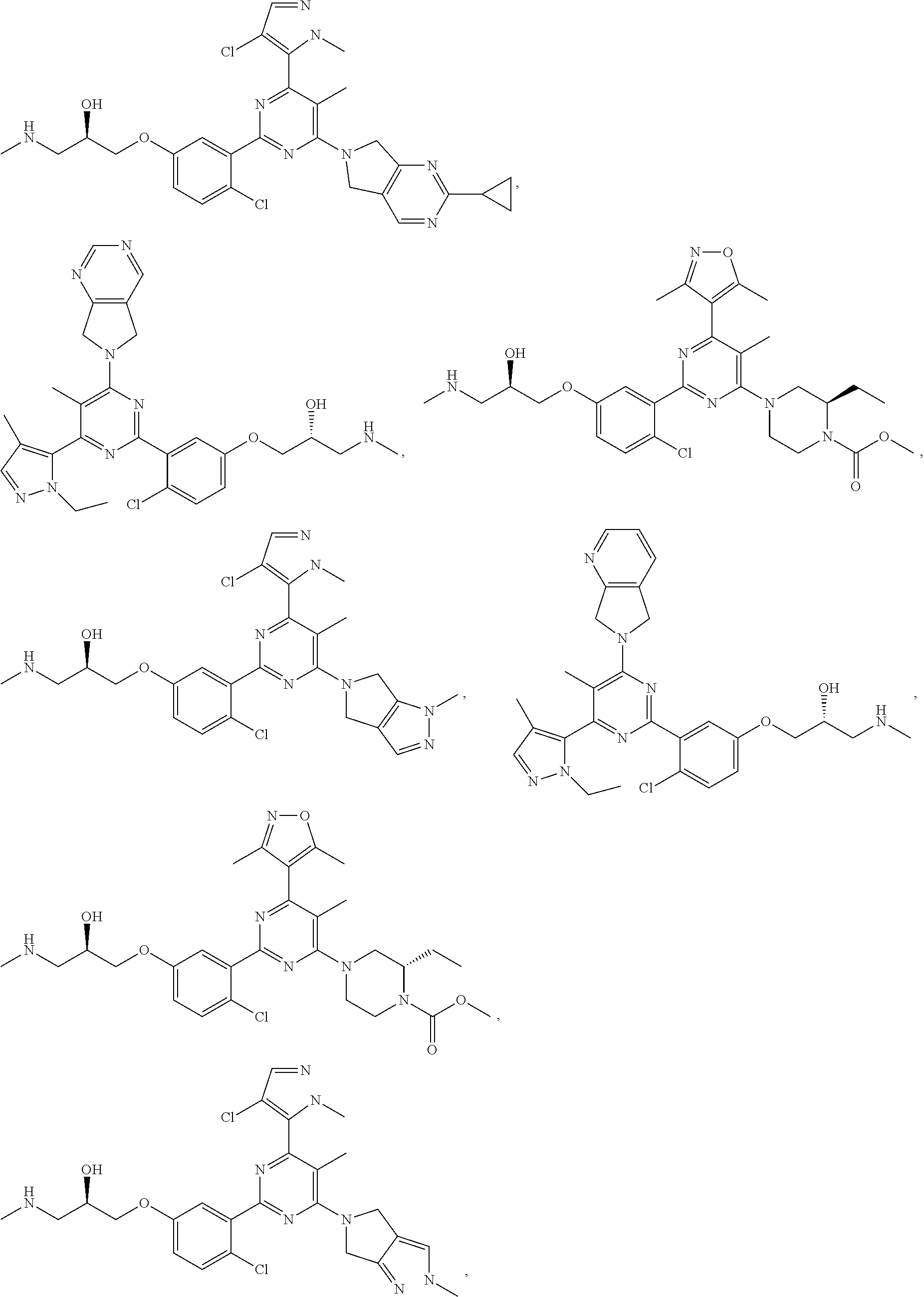 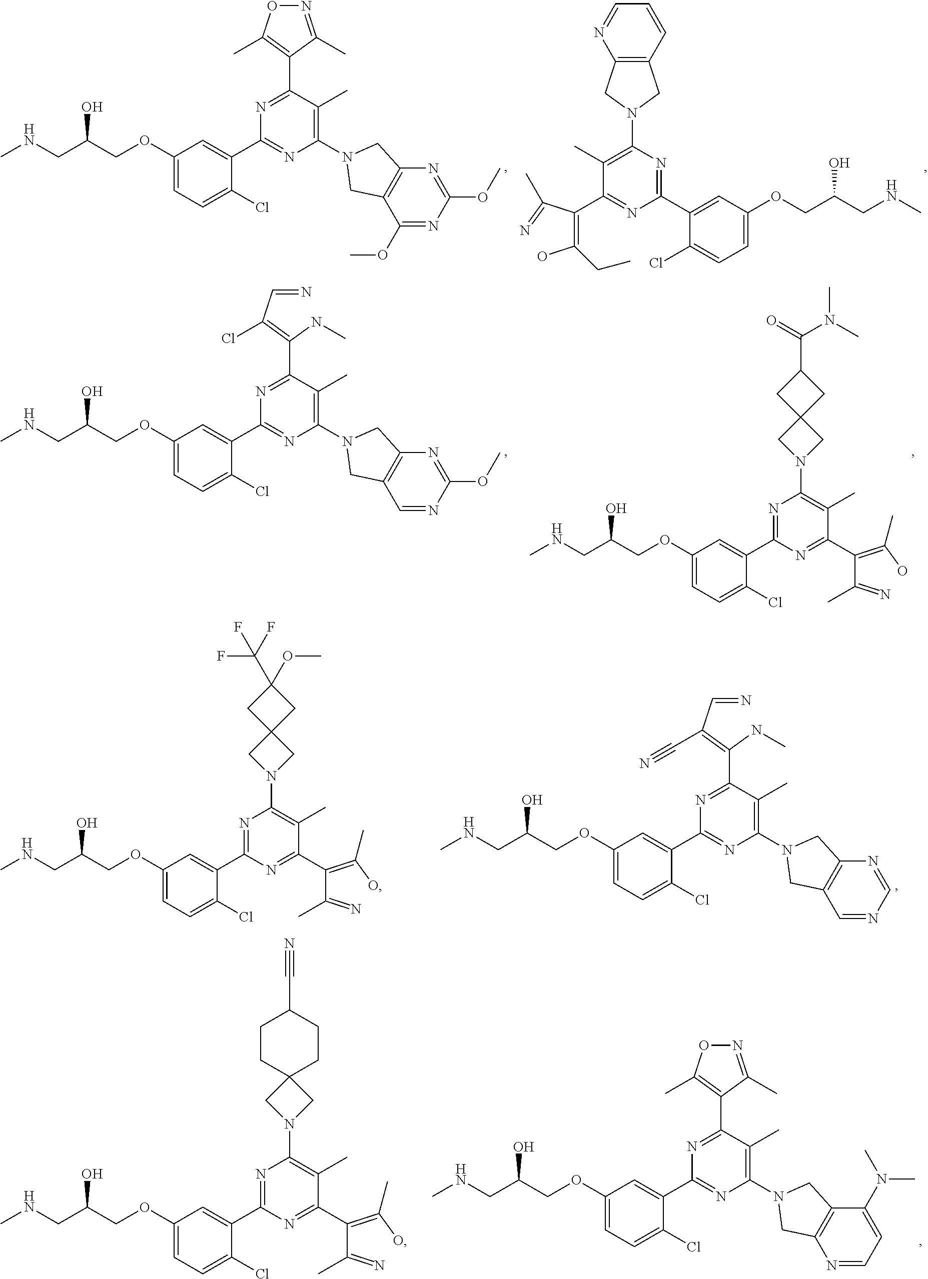 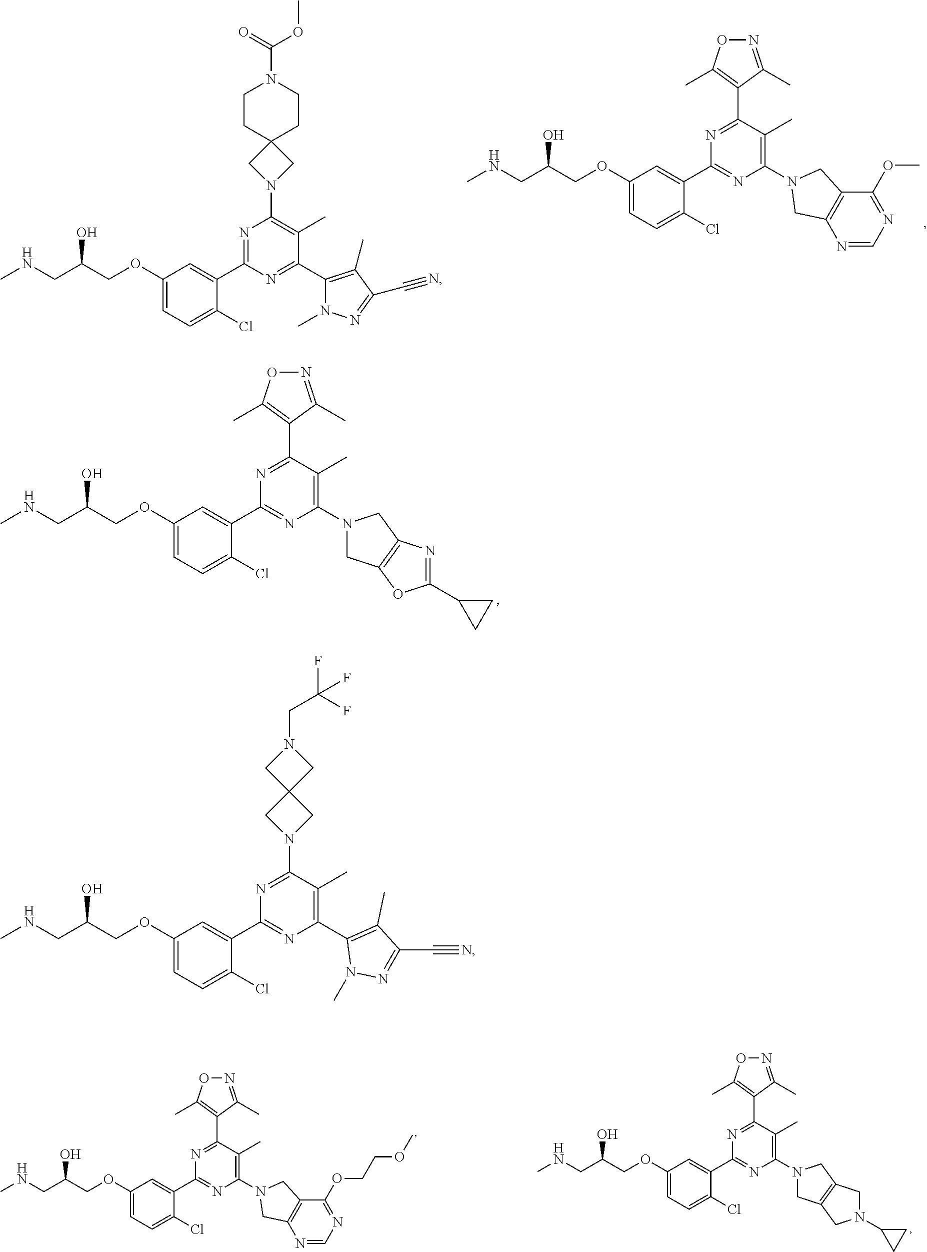 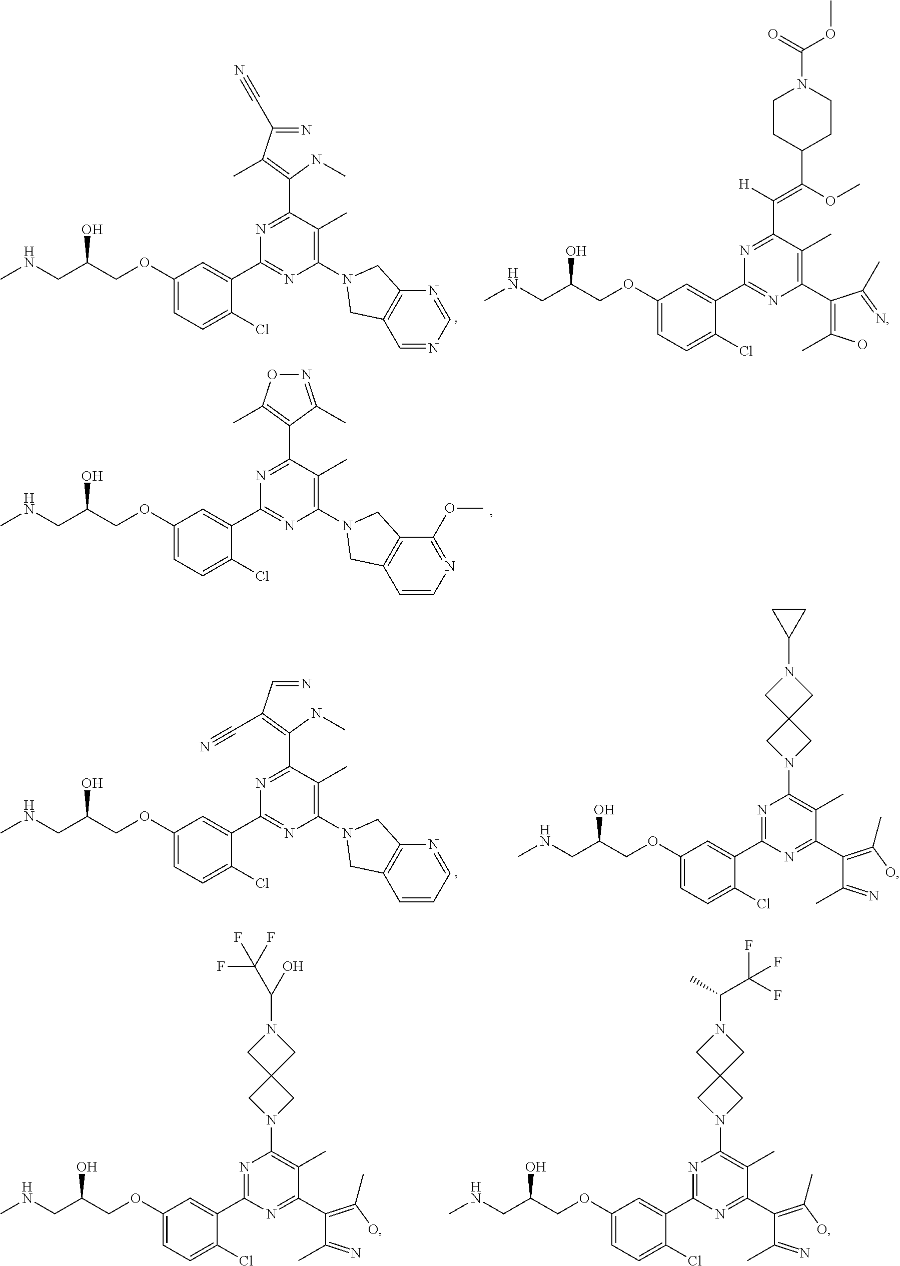 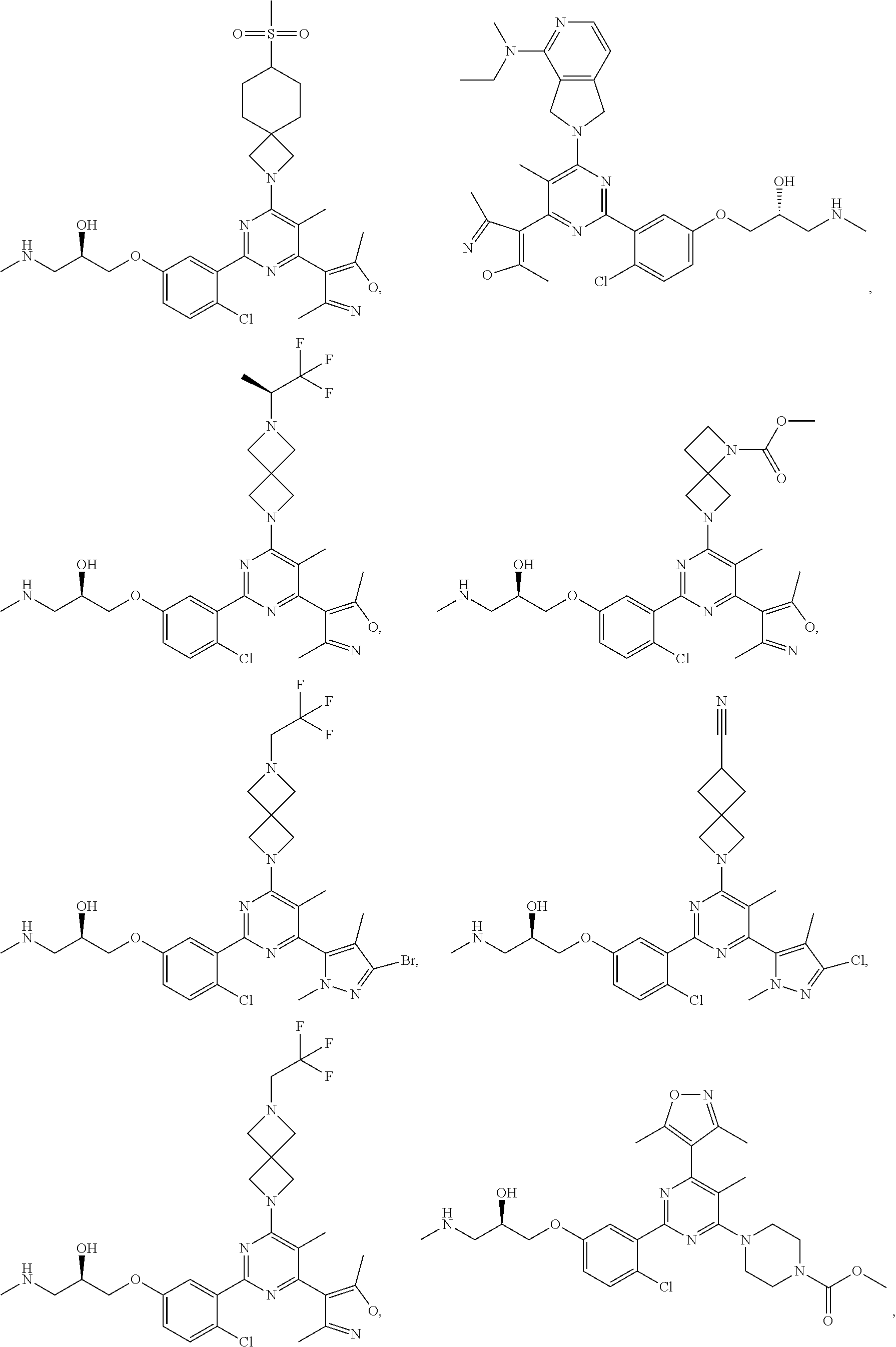 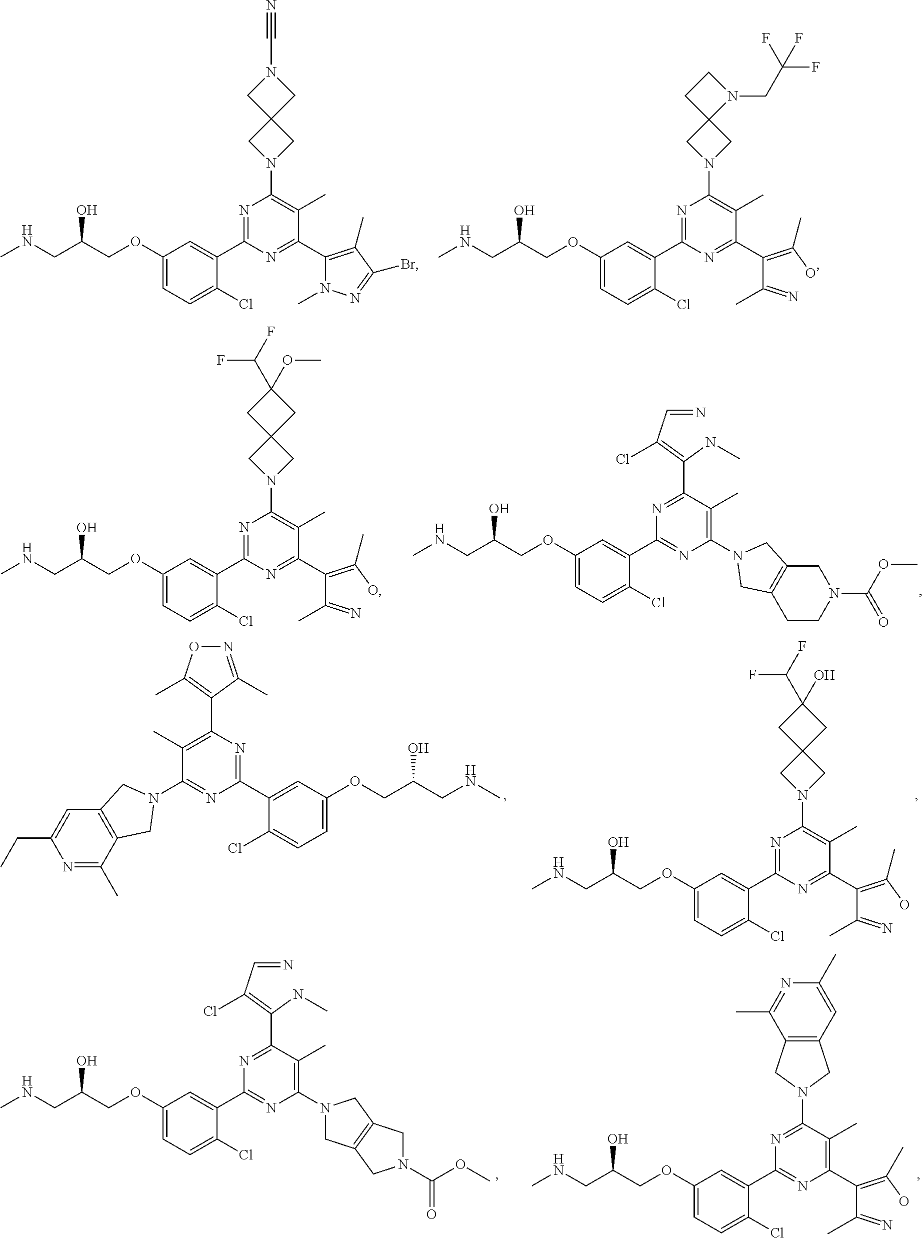 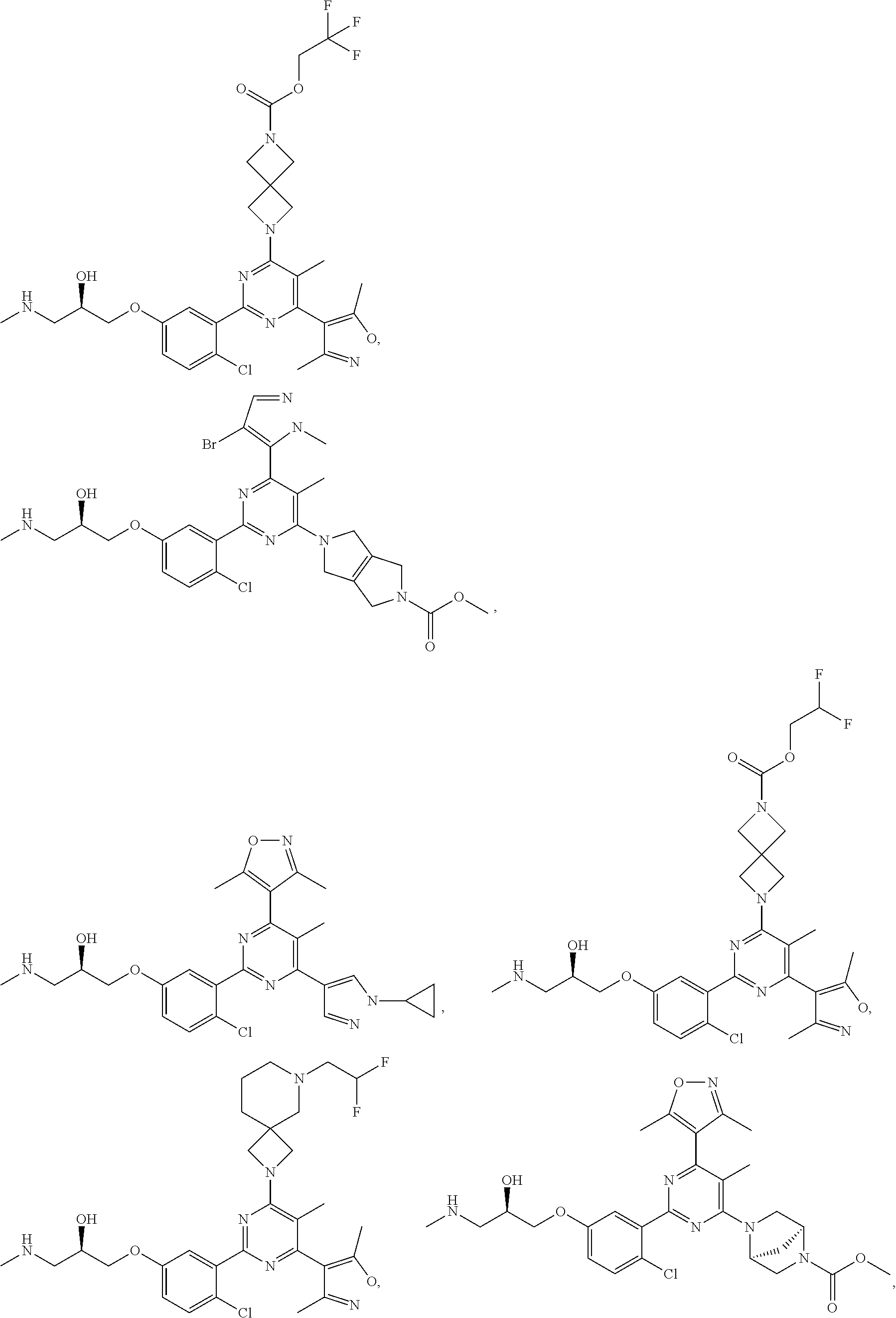 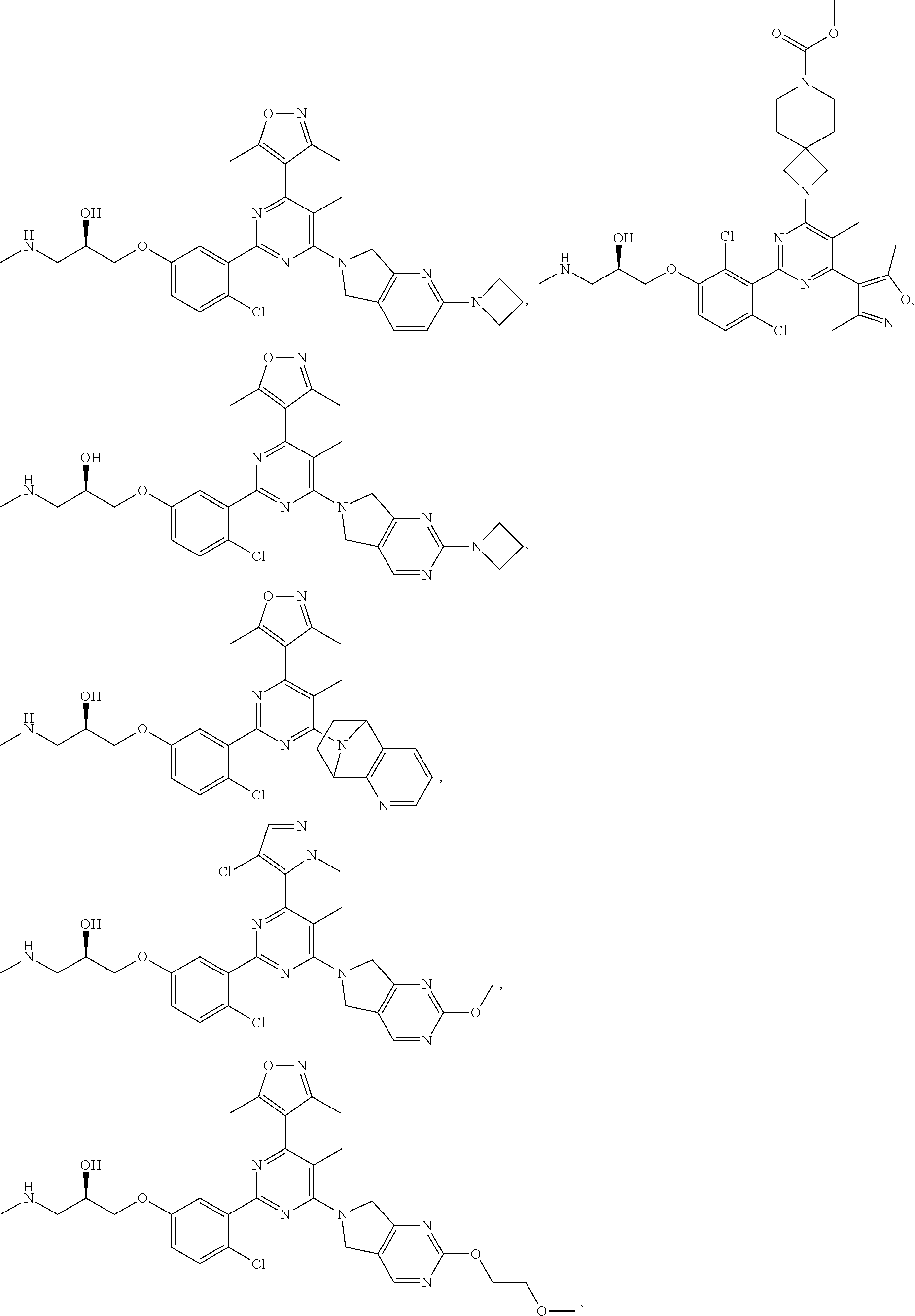 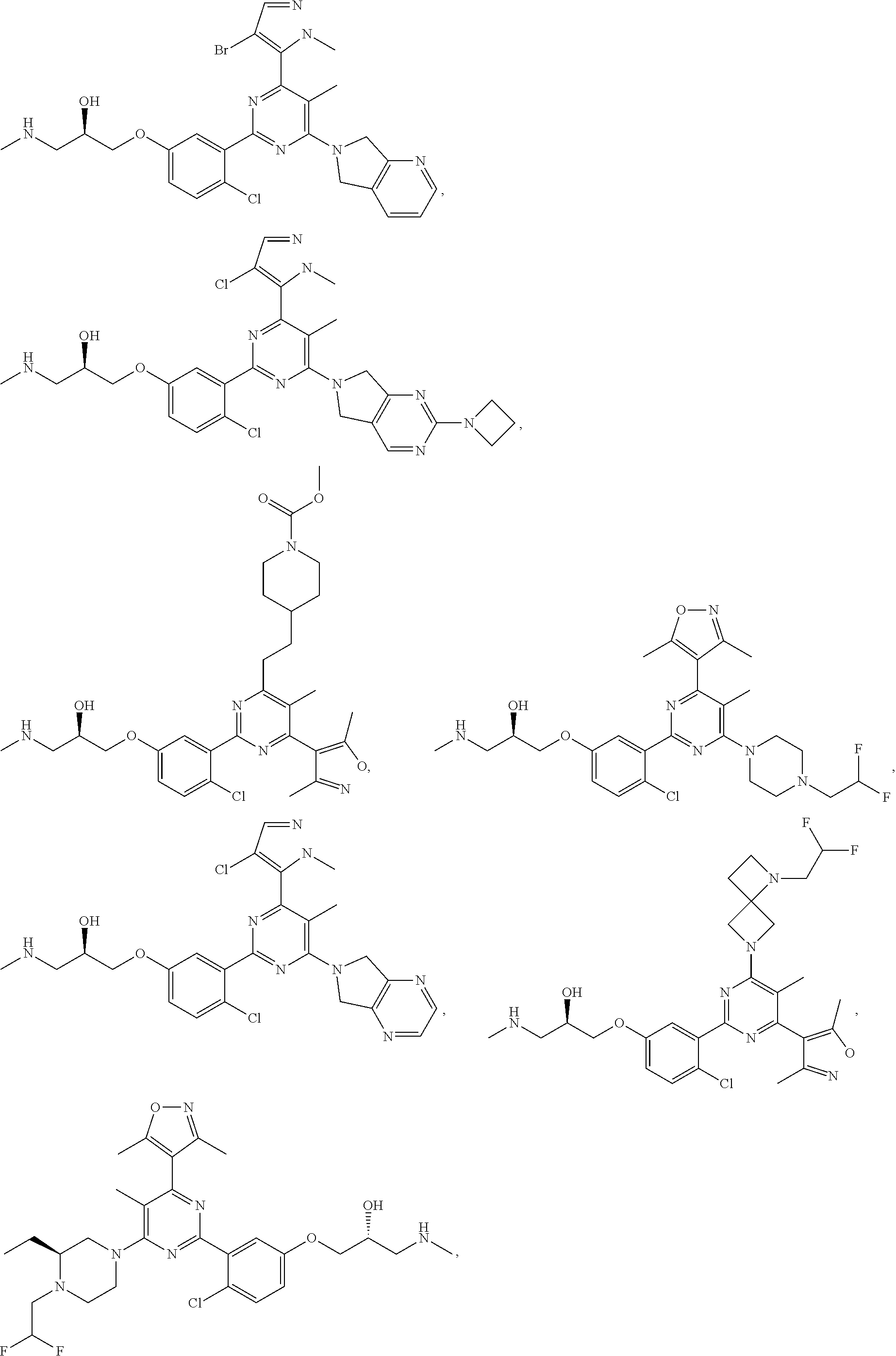 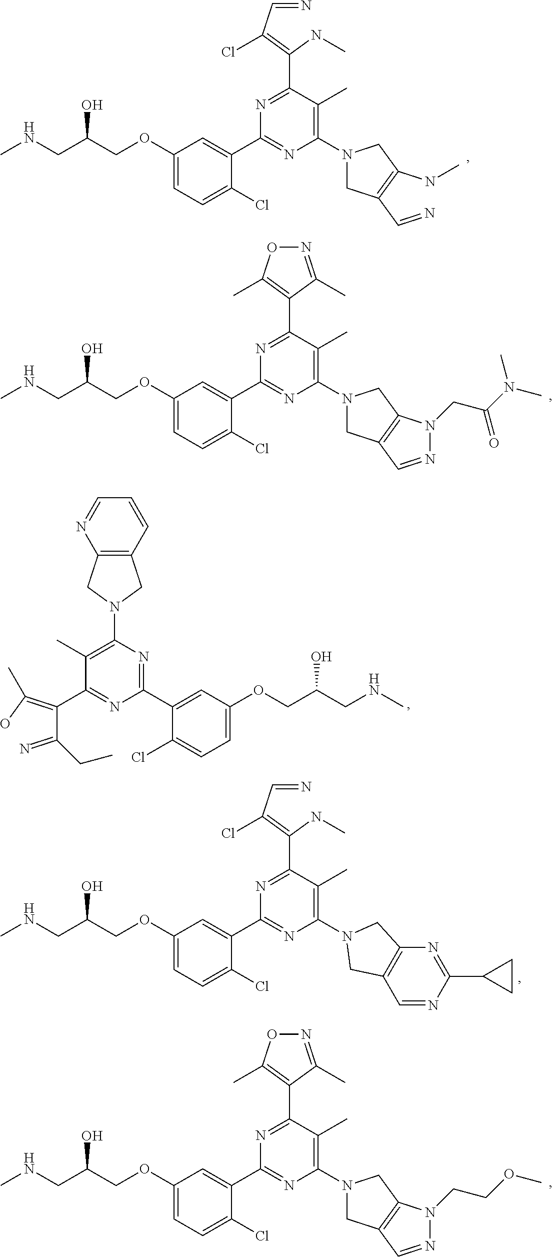 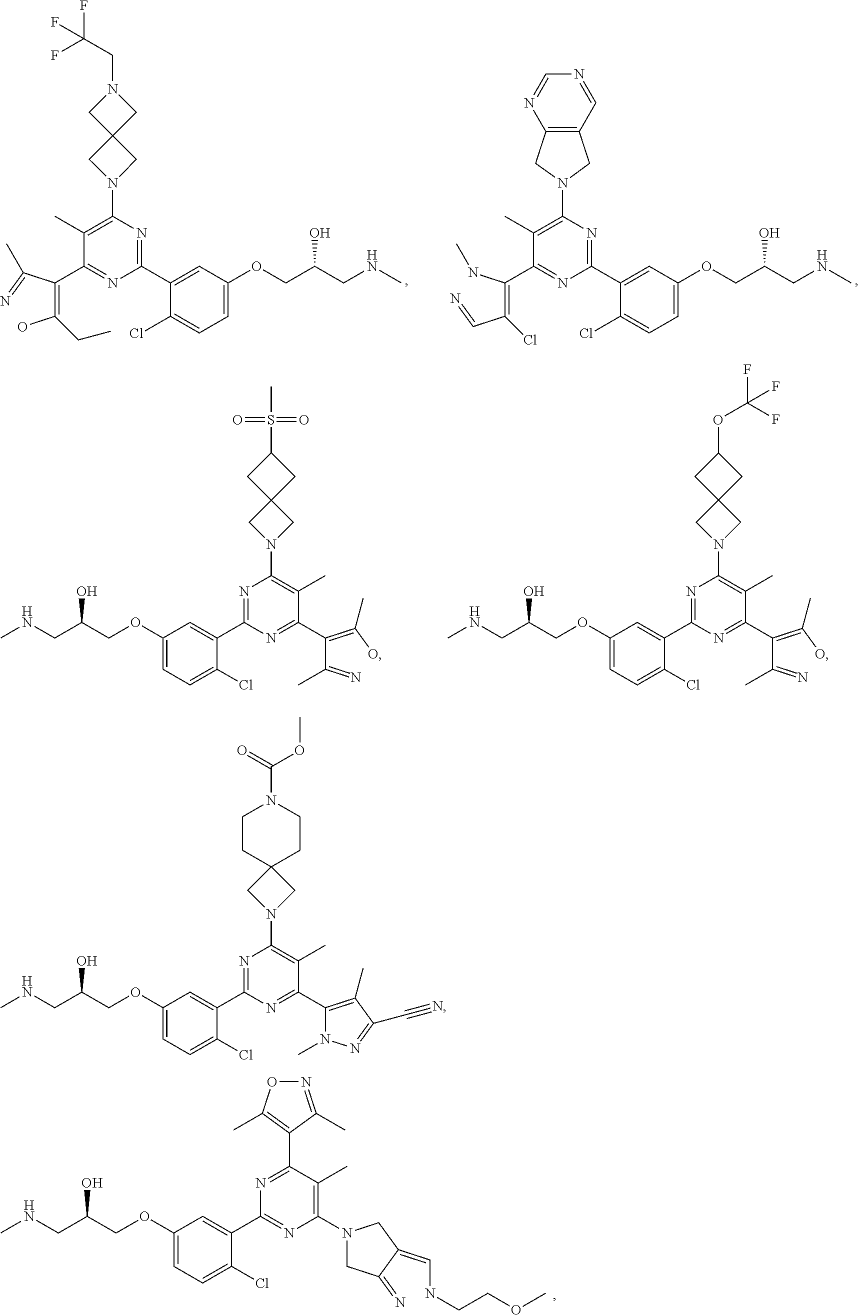 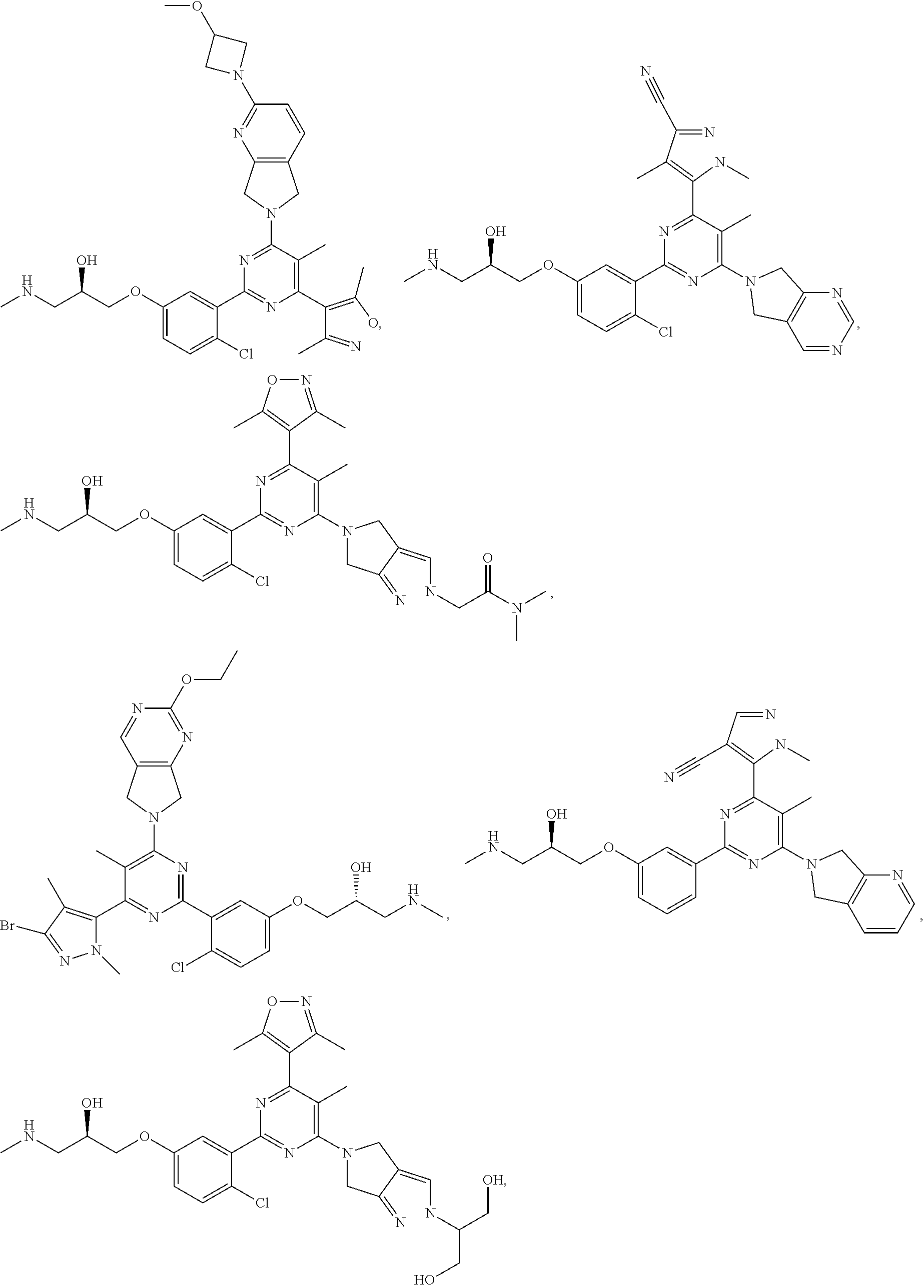 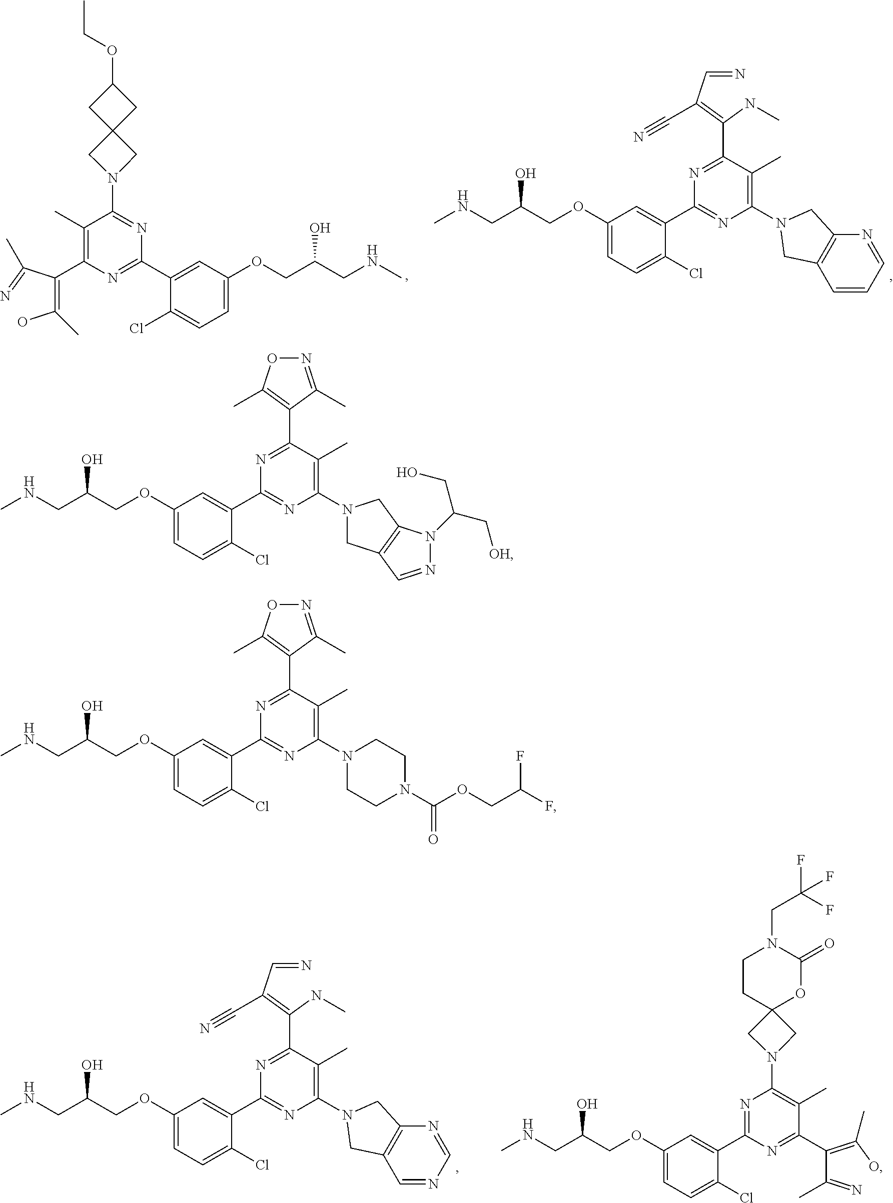 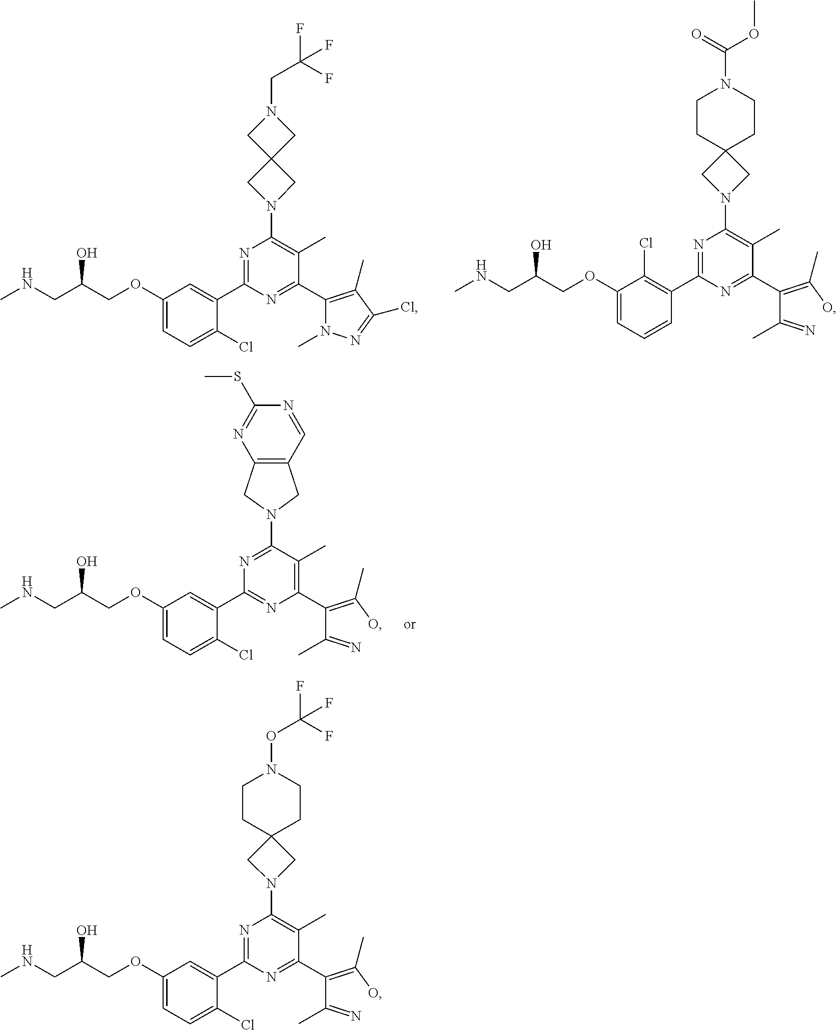
or a pharmaceutically acceptable salt thereof.

162. The compound of claim 1 which is: 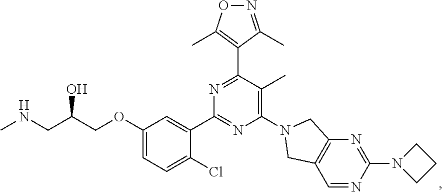 [SMILES: CNC[C@@H](O)COc1ccc(Cl)c(-c2nc(-c3c(C)noc3C)c(C)c(N3Cc4cnc(N5CCC5)nc4C3)n2)c1]
or a pharmaceutically acceptable salt thereof.

163. The compound of claim 1 which is: 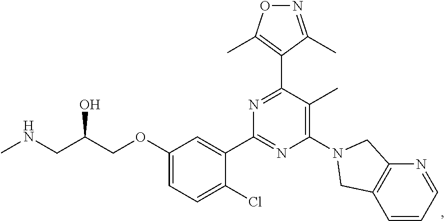 [SMILES: CNC[C@@H](O)COc1ccc(Cl)c(-c2nc(-c3c(C)noc3C)c(C)c(N3Cc4cccnc4C3)n2)c1]
or a pharmaceutically acceptable salt thereof.

164. A pharmaceutical composition comprising the compound of claim 162 or a pharmaceutically acceptable salt thereof, and a pharmaceutically acceptable excipient.

165. A pharmaceutical composition comprising the compound of claim 163 or a pharmaceutically acceptable salt thereof, and a pharmaceutically acceptable excipient.


SEQUENCE LISTING (4 sequences)

SEQ ID NO: 1 - protein, 15 aa, Artificial Sequence
  Synthetic Polypeptide
PRKQLATKAARKSAP

SEQ ID NO: 2 - protein, 9 aa, Artificial Sequence
  Synthetic Polypeptide
MDYKDDDDK

SEQ ID NO: 3 - protein, 8 aa, Artificial Sequence
  Synthetic Polypeptide
EGHHHHHH

SEQ ID NO: 4 - protein, 601 aa, Artificial Sequence
  Synthetic Polypeptide
MDYKDDDDKAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHRIEIPFKFHMLHSGLVHGLAFWFDVAFIGSIMTVWLSTAPTEPLTHWYQVRCLFQSPLFAKAGDTLSGTCLLIANKRQSYDISIVAQVDQTGSKSSNLLDLKNPFFRYTGTTPSPPPGSHYTSPSENMWNTGSTYNLSSGMAVAGMPTAYDLSSVIASGSSVGHNNLIPLGSSGAQGSGGGSTSAHYAVNSQFTMGGPAISMASPMSIPTNTMHYGSEGHHHHHH